(19) United States
(12) Patent Application Publication
(10) Pub. No.: US 2022/0281876 A1
(43) Pub. Date: Sep. 8, 2022

(54) DEGRADATION OF BRUTON'S TYROSINE KINASE (BTK) BY CONJUGATION OF BTK INHIBITORS WITH E3 LIGASE LIGAND AND METHODS OF USE

(71) Applicant: BEIGENE, LTD., Camana Bay, Grand Cayman (KY)

(72) Inventors: Hexiang WANG, Beijing (CN); Changxin HUO, Beijing (CN); Yunhang GUO, Beijing (CN); Ruipeng QI, Beijing (CN); Zhiwei WANG, Beijing (CN)

(21) Appl. No.: 17/629,910

(22) PCT Filed: Jul. 24, 2020
(86) PCT No.: PCT/CN2020/103988

§ 371 Date: Jan. 25, 2022

(30) Foreign Application Priority Data
Jul. 26, 2019 (CN) PCT/CN2019/098015

(51) Int. Cl. C07D 487/04; C07D 519/00

(52) U.S. Cl. CPC C07D 487/04; C07D 519/00

(57) ABSTRACT

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis-targeting chimera (PROTAC) is a novel strategy for selective knockdown of target proteins by small molecules (Sakamoto K M et al., Proc Natl Acad Sci 2001, 98:8554-9.; Sakamoto K. M. et al., Methods Enzymol. 2005; 399:833-847.). PROTAC utilizes the ubiquitin-protease system to target a specific protein and induce its degradation in the cell (Zhou P. et al., Mol Cell. 2000; 6(3):751-756; Neklesa T. K. et al., Pharmacol Ther. 2017; 174:138-144; Lu M. et al., Eur J Med Chem. 2018; 146:251-259;). The normal physiological function of the ubiquitin-protease system is responsible for clearing denatured, mutated, or harmful proteins in cells. The normal physiological function of the ubiquitin-protease system is responsible for clearing denatured, mutated, or harmful proteins in cells. The ubiquitin-proteasome system (UPS), also known as the ubiquitin-proteasome pathway (UPP), is a common posttranslational regulation mechanism that is responsible for protein degradation in normal and pathological states (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Ubiquitin, which is highly conserved in eukaryotic cells, is a modifier molecule, composed of 76 amino acids, that covalently binds to and labels target substrates via a cascade of enzymatic reactions involving E1, E2, and E3 enzymes. Subsequently, the modified substrate is recognized by the 26S proteasome complex for ubiquitination-mediated degradation. So far, two E1 enzymes have been discovered, which are termed UBA1 and UBA6. On the other hand, there are about 40 E2 enzymes and more than 600 E3 enzymes that offer the functional diversity to govern the activity of many downstream protein substrates. However, only a limited number of E3 ubiquitin ligases have been successfully hijacked for use by small molecule PROTAC technology: the Von Hippel-Lindau disease tumor suppressor protein (VHL), the Mouse Double Minute 2 homologue (MDM2), the Cellular Inhibitor of Apoptosis (cIAP), and cereblon (Philipp O. et al., Chem. Biol. 2017, 12, 2570-2578).

Bifunctional compounds composed of a target protein-binding moiety and an E3 ubiquitin ligase-binding moiety have been shown to induce proteasome-mediated degradation of selected proteins. These drug-like molecules offer the possibility of temporal control over protein expression, and could be useful as biochemical reagents for the treatment of diseases. In recent years, this newly developed method has been widely used in antitumor studies (Lu J. et al., Chem Biol. 2015; 22(6):755-763; Ottis P. et al., Chem Biol. 2017; 12(4):892-898.; Crews C. M. et al., J Med Chem. 2018; 61(2):403-404; Neklesa T. K. et al., Pharmacol Ther. 2017, 174:138-144.; Cermakova K. et al., Molecules, 2018.23(8).; An S. et al., EBioMedicine, 2018.; Lebraud H. et al., Essays Biochem. 2017; 61(5): 517-527.; Sun Y. H. et al., Cell Res. 2018; 28:779-81; Toure M. et al., Angew Chem Int Ed Engl. 2016; 55(6):1966-1973;); and has been disclosed or discussed in patent publications, e.g., US20160045607, US20170008904, US20180050021, US20180072711, WO2002020740, WO2014108452, WO2016146985, WO2016149668, WO2016149989, WO2016197032, WO2016197114, WO2017011590, WO2017030814, WO2017079267, WO2017182418, WO2017197036, WO2017197046, WO2017197051, WO2017197056, WO2017201449, WO2017211924, WO2018033556, and WO2018071606.

Bruton's tyrosine kinase (Btk) belongs to the Tec tyrosine kinase family (Vetrie et al., Nature 361: 226-233, 1993; Bradshaw, Cell Signal. 22: 1175-84, 2010). Btk is primarily expressed in most hematopoietic cells such as B cells, mast cells and macrophages (Smith et al., J. Immunol. 152: 557-565, 1994) and is localized in bone marrow, spleen and lymph node tissue. Btk plays important roles in B-cell receptor (BCR) and FcR signaling pathways, which involve in B-cell development, differentiation (Khan, Immunol. Res. 23: 147, 2001). Btk is activated by upstream Src-family kinases. Once activated, Btk in turn phosphorylates PLC gamma, leading to effects on B-cell function and survival (Humphries et al., J. Biol. Chem. 279: 37651, 2004). These signaling pathways must be precisely regulated. Mutations in the gene encoding Btk cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that Btk deficient mice are resistant to developing collagen-induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, Btk inhibitors can be used to treat autoimmune and/or inflammatory diseases.

Inhibition of BTK has been shown to affect cancer development (B cell malignancies) and cell viability, and improve autoimmune diseases (e.g., rheumatoid arthritis and lupus). Inhibition of BTK has also been reported via alternative strategies, such as through degradation of BTK (Alexandru D. et al., Biochemistry 2018, 57, 26, 3564-3575; Adelajda Z. et al., PNAS 2018 115 (31); Dennis D., et al., Blood, 2019, 133:952-961; Yonghui S. et al., Cell Research, 2018, 28, 779-781; Yonghui S. et al., Leukemia, 2019, Degradation of Bruton's tyrosine kinase mutants by PROTACs for potential treatment of ibrutinib-resistant non-Hodgkin lymphomas).

There is a need of new BTK inhibitors which are more potent than known inhibitors of BTK and inhibit BTK via alternative strategies, such as through degradation of BTK. The present application addresses the need.

### SUMMARY OF THE INVENTION

One objective of the present invention is to provide a proteolysis targeting chimera (PROTAC) compound by conjugating a BTK inhibitor with an E3 ligase ligand, which function to recruit targeted proteins to E3 ubiquitin ligase for degradation, and to provide a method of the preparation and uses thereof. In particular, the present disclosure provides PROTAC compounds with the Formula I.

Aspect 1: A compound of Formula (I):

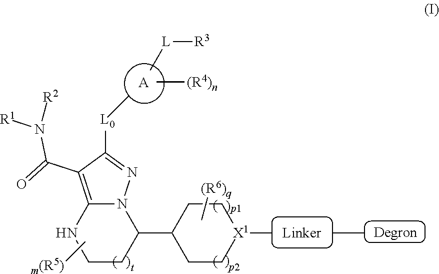

or a pharmaceutically acceptable salt thereof, or a stereoisomer thereof,
wherein:

A is a 5- or 6-membered aromatic ring comprising 0-3 heteroatoms selected from N, S and O;

L and L₀ are each independently a bond, —CH₂—, —NR⁷—, —O—, or —S—;

m, n and q are each independently 0, 1, 2, 3 or 4;

t is 0, 1 or 2;

p1 and p2 are each independently 0, 1 or 2;

R¹, R², and R⁷ are each independently hydrogen, —C₁₋₈alkyl, —C₂₋₈ alkenyl, —C₂₋₈ alkynyl, cycloalkyl, heterocyclyl, aryl, or heteroaryl, each of said —C₁₋₈ alkyl, —C₂₋₈ alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;

R³, R⁴, R⁵, and R⁶ are each independently hydrogen, halogen, —C₁₋₈alkyl, —C₂₋₈ alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;

R^{a}, R^{b}, and R^{c} are each independently hydrogen, —C₁₋₈alkyl, —C₂₋₈alkenyl, —C₂₋₈alkynyl, cycloalkyl, heterocyclyl, aryl, or heteroaryl;

or R¹ and R², together with the nitrogen atom to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈ alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said —C₁₋₈alkyl, —C₂₋₈alkenyl, —C₂₋₈ alkynyl, cycloalkyl, heterocyclyl, aryl, or heteroaryl is optionally substituted with at least one substituent selected from halogen, —C₁₋₈ alkyl, —OR³ⁱ, —NR³ⁱR^{3j}, cycloalkyl, heterocyclyl, aryl, or heteroaryl;

R^{3f}, R^{3g}, R^{3h}, R³ⁱ, and R^{3j} are each independently hydrogen, —C₁₋₈alkyl, C₁₋₈alkoxy-C₁₋₈alkyl-, —C₂₋₈alkenyl, —C₂₋₈ alkynyl, cycloalkyl, heterocyclyl, aryl, or heteroaryl;

X₁ is selected from —CH— or N;

the Linker is a bond or a divalent linking group, and

the Degron is an E3 Ubiquitin ligase moiety.

Aspect 2: The compound according to Aspect 1, wherein the Degron moiety is selected from Formulas D1, D2, D3, D4 or D5:

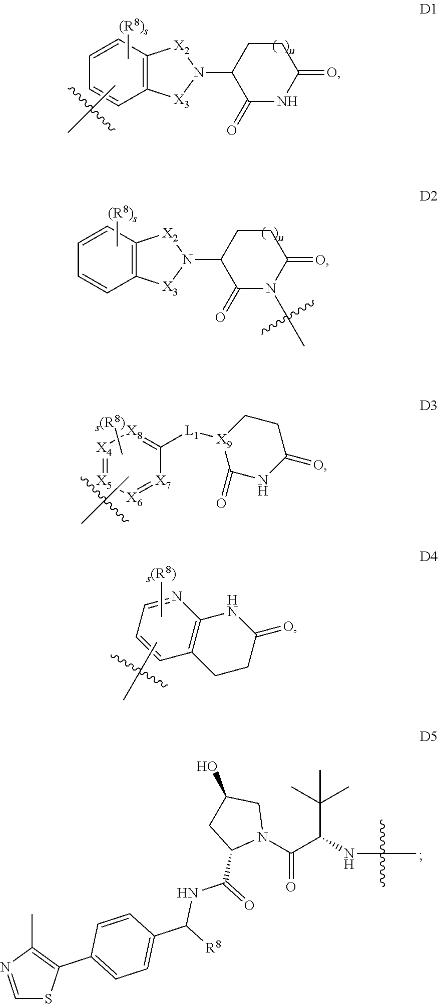

wherein

X₂ and X₃ are each independently —CH₂—, —NH— or —C(O)—;

X₄, X₅, X₆, X₇ and X₈ are each independently CH or N;

X₉ is CH or N;

L₁ is selected from a bond, —CH₂—, —O—, —NH— and —S—;

s is 0, 1, 2, 3, or 4;

u is 0, 1, or 2;

R⁸ is each independently hydrogen, halogen, —C₁₋₈alkyl, —C₂₋₈ alkenyl, —C₂₋₈ alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8c}, —NR^{8a}R^{8b}—NR^{8a}COR^{8b}, —NR^{8a}CONR^{8b}R^{8c}, —NR^{8a}CO₂R^{8b}, —NR^{8a}SONR^{8b}R^{8c}, —NR^{8a}SO₂NR^{8b}R^{8c}, or —NR^{8a}SO₂R^{8b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈ alkyoxy, cycloalkyl, heterocyclyl, aryl, or heteroaryl;

R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl;

wherein the Degron moiety binds to the linker via

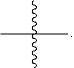

Aspect 3: The compound according to Aspect 1, wherein Formula D1 is selected from

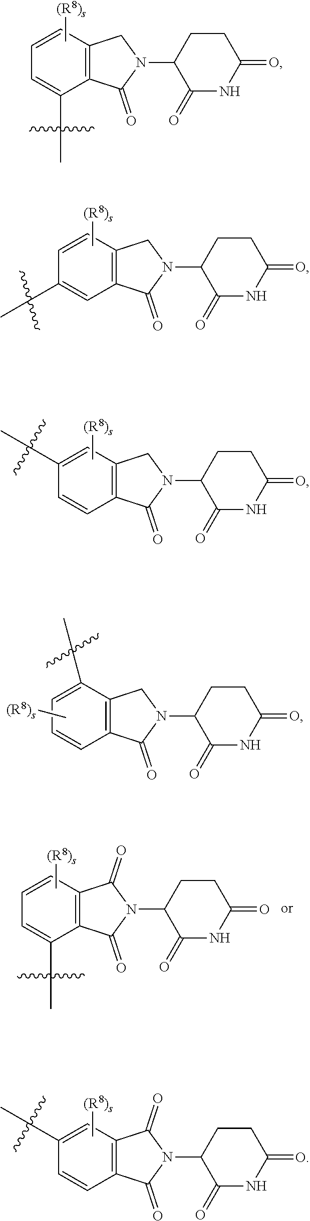

Aspect 4: The compound according to Aspect 1, wherein Formula D2 is selected from

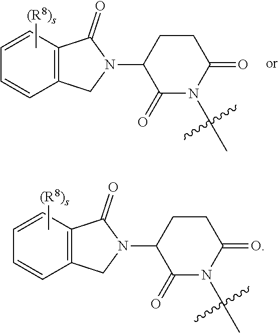

Aspect 5: The compound according to Aspect 4, wherein Formula D2 is selected from

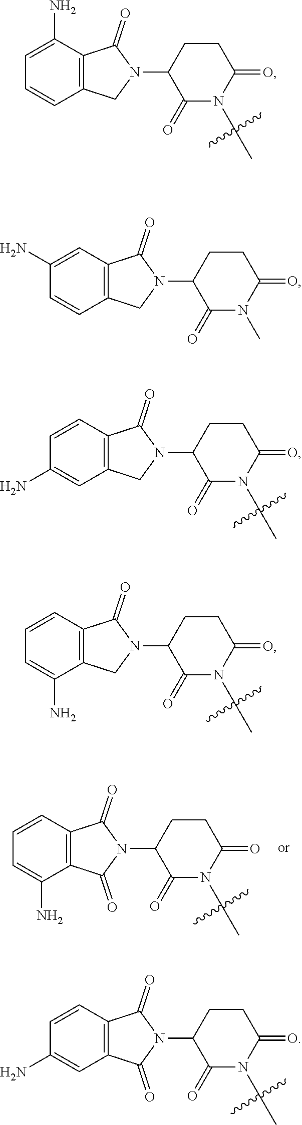

Aspect 6: The compound according to Aspect 2, wherein Formula D3 is selected from

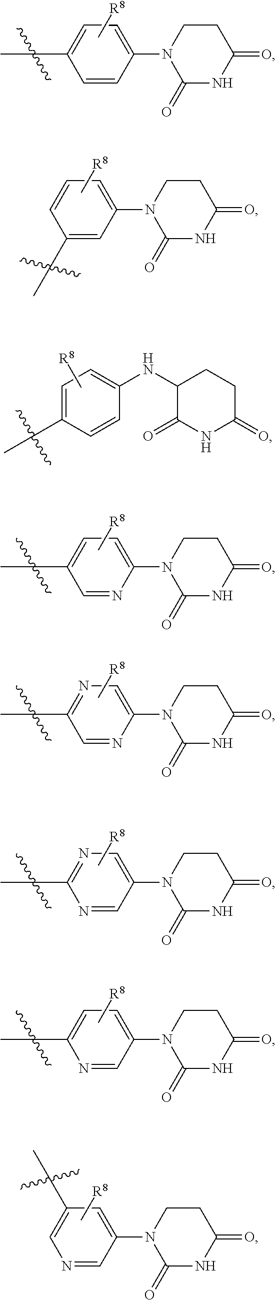

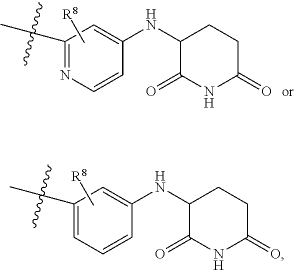

wherein R⁸ is defined as above.

Aspect 7: The compound according to Aspect 6, wherein Formula D3 is selected from

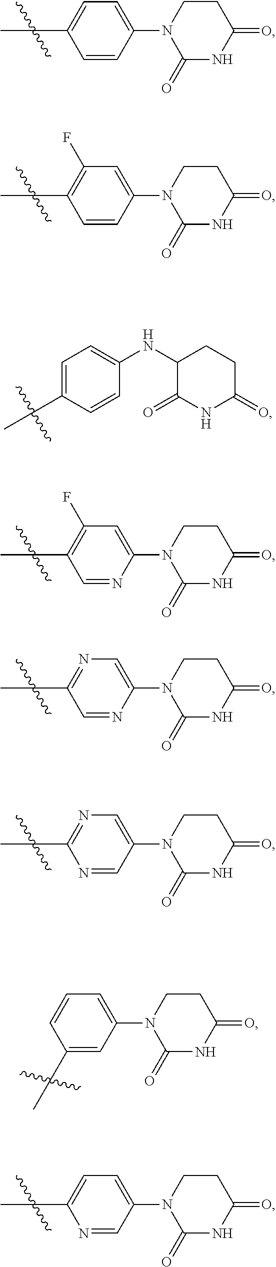

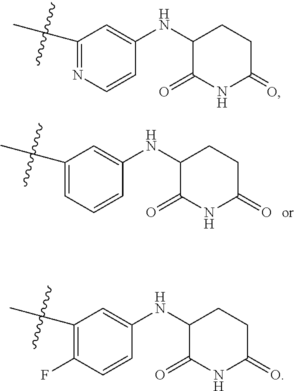 [SMILES: *c1cc(NC2CCC(=O)NC2=O)ccn1 ; *c1cccc(NC2CCC(=O)NC2=O)c1 ; *c1cc(NC2CCC(=O)NC2=O)ccc1F]

Aspect 8: The compound according to Aspect 2, wherein Formula D4 is selected from

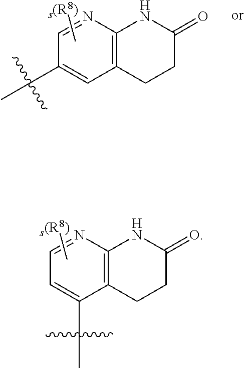

Aspect 9: The compound according to Aspect 8, wherein Formula D4 is selected from

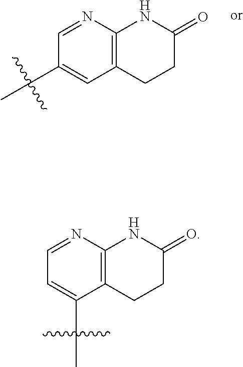 [SMILES: *c1cnc2c(c1)CCC(=O)N2 ; "or" ; *c1ccnc2c1CCC(=O)N2]

Aspect 10: The compound according to any one of Aspects 1-9, wherein A is phenyl; p=1, q=0, m=0, and t=1.

Aspect 11: The compound according to any one of Aspects 1-10, wherein L is O or NH.

Aspect 12: The compound according to any one of Aspects 1-11, wherein R¹ and R² are both H.

Aspect 13: The compound according to any one of Aspects 1-11, wherein

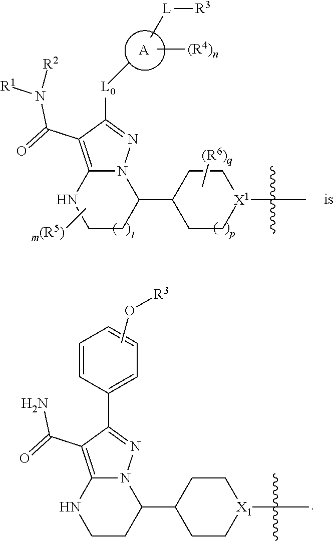

Aspect 14: The compound according to Aspect 13, wherein

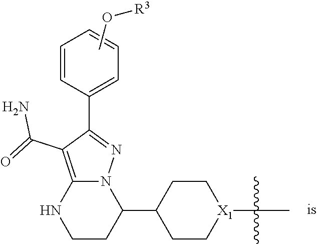

Aspect 15: The compound according to Aspect 14, wherein

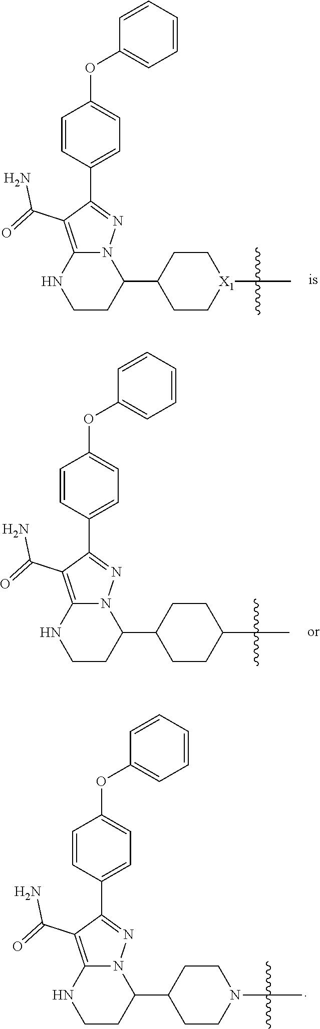

Aspect 16: The compound according to Aspect 15, wherein

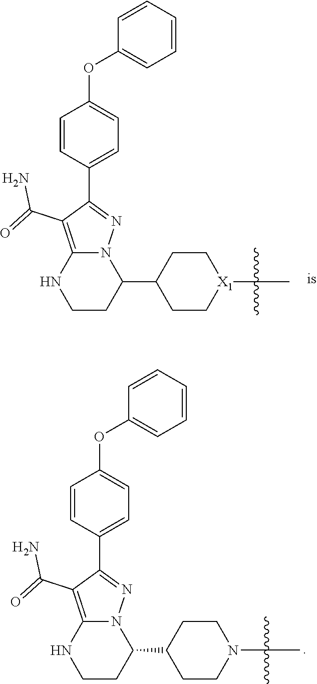

Aspect 17: The compound according to any one of Aspects 1-16, where in the Linker is selected from

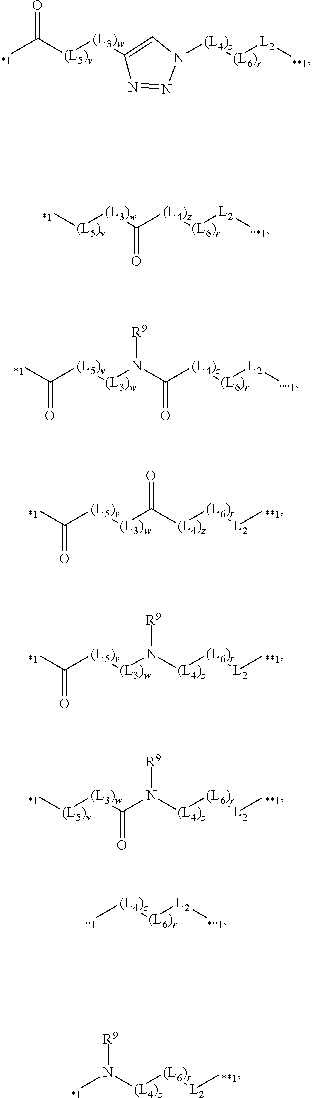

wherein *1 refers to the position attached to

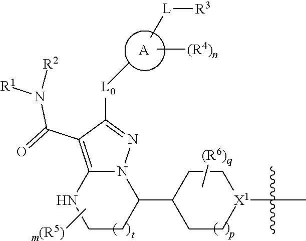

(sometimes referred to as the BTK moiety), and **1 refers to the position attached to the Degron;

r, v, w, and z are each independently 0, 1, 2, 3, 4, 5, 6, 7, 8, 9 or 10;

L₂ is —CH₂—, —NH—, —O—, —C(O)—, —NHC(O)—,

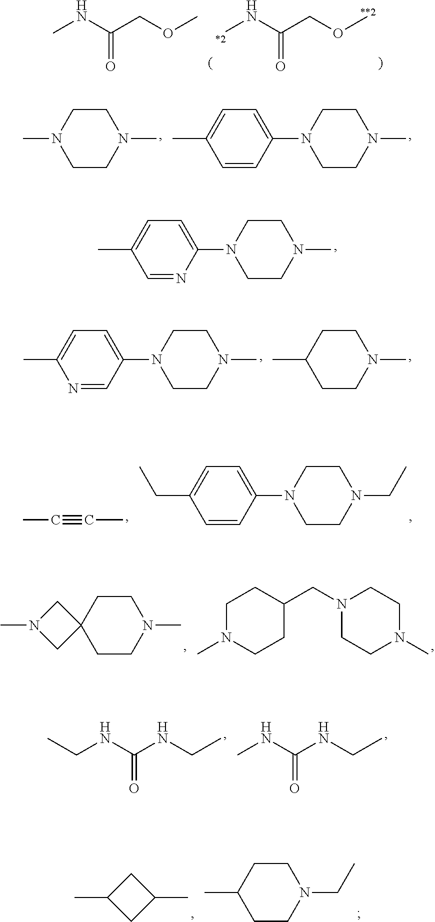

wherein *2 refers to the position attached to L₄ and **2 refers to the position attached to the Degron;

L₃, L₄, L₅ and L₆ are each independently —CH₂—, —CH₂CH₂—, —OCH₂CH₂—, —CH₂—O—CH₂—, —CH₂CH₂O—, —C(O)—, —NHC(O)—, —CH₂—CONH—,

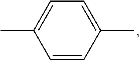

### —CH═CH—,

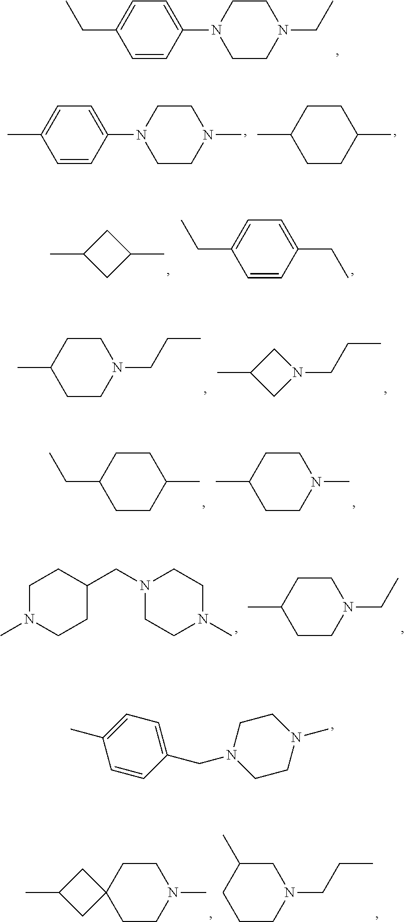

R⁹ is selected from H or CH₃.

Aspect 18: The compound according to any one of Aspects 1-17, wherein the Linker is selected from

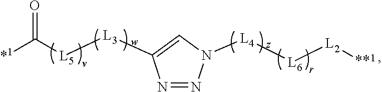

v=0; w=0, 1, 2, 3, or 4; L₃ is —CH₂—; L₄ is —CH₂CH₂O— or —CH₂—; z=0, 1, 2, 3, 4, 5, 6, or 7; L₆ is —CH₂— or —NHC(O)—; r=0, 1, 2, 3, or 4; L₂ is —NH—, —CH₂—, —O— or —C═C—.

Aspect 19: The compound according to Aspect 17, wherein v=0; W=0; L₄ is —CH₂CH₂O—; z=1, 2, 3, 4, 5, 6, or 7; L₆ is —CH₂—; r=0, 1, 2, or 3; L₂ is —NH—, or —CH₂—.

Aspect 20: The compound according to Aspect 18, wherein v=0; w=0; L₄ is —CH₂CH₂O—; z=1, 2, 3; L₆ is —CH₂—; r=1, 2, or 3; L₂ is —C═C—.

Aspect 21: The compound according to Aspect 17, wherein v=0, L₃ is —CH₂—, w=2 or 3, L₄ is —CH₂CH₂O— or —CH₂—, z=1, 2, 3, or 4; L₆ is —CH₂—; r=1, 2, or 3; L₂ is —NH—, or —CH₂—.

Aspect 22: The compound according to Aspect 17, wherein V=0, L₃ is w=2 or 3, L₄ is —CH₂—, z=3, 4 or 5; r=0; L₂ is

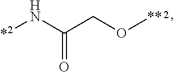

wherein *2 refers to the position attached to L₄ and **2 refers to the position attached to the Degron.

Aspect 23: The compound according to Aspect 17, wherein the Linker is selected from

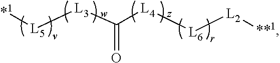

wherein

L₅ is —CH₂CH₂O—, or

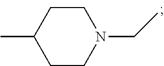

v=0, 1, 2 or 3, L₃ is —CH₂— or

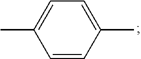

w=0, 1, 2, or 3; L₄ is —CH₂—O—CH₂—, —CH₂—,

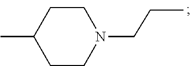

z=0, 1, 2, 3, 4, 5, or 6; L₆ is —CH₂—, —OCH₂CH₂—,

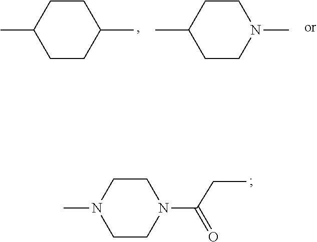

r=0, 1, 2, 3, 4, 5, 6, 7 or 8; L₂ is —NH—,

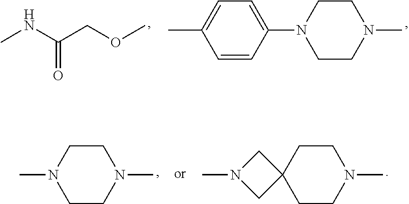

Aspect 24: The compound according to Aspect 23, wherein L₅ is —CH₂CH₂O—; v=1, 2 or 3, L₃ is —CH₂—; w=1; Z=0; r=0; L₂ is —NH—.

Aspect 25: The compound according to Aspect 23, wherein v=w=0; L₄ is —CH₂—O—CH₂—; z=1, 2, 3 or 4; L₆ is —CH₂—; r=1, 2, 3, 4, 5, 6, 7 or 8; L₂ is —NH— or

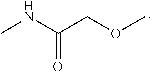

Aspect 26: The compound according to Aspect 23, wherein v=w=z=0; L₆ is —CH₂—; r=2, 3, 4, 5, or 6;

L₂ is —NH— or

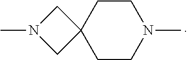

Aspect 27: The compound according to Aspect 23, wherein v=w=0; L₄ is

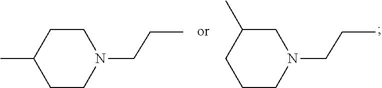

z=1; L₆ is —OCH₂CH₂—; r=1, 2, 3; L₂ is —NH—.

Aspect 28: The compound according to Aspect 17, wherein the Linker is selected from

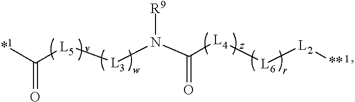

wherein

L₅ is —CH₂CH₂O—, —CH₂— or —CH₂—O—CH₂—;

v=1, 2, 3 or 4, L₃ is —CH₂—,

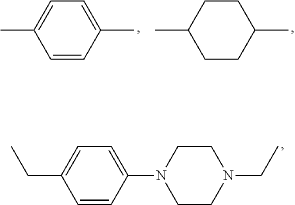

or —CH₂CH₂O—;

w=0, 1, 2 or 3;

R⁹ is H or CH₃;

L₄ is —CH₂— or —CH₂—O—CH₂—;

z=0, 1, 2, 3, or 4; L₆ is —CH₂—;

r=0, 1, 2, 3, or 4; L₂ is —NH—, —CH₂—, —O—.

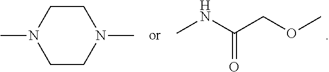

Aspect 29: The PROTAC compound according to Aspect 28, wherein

L₅ is —CH₂—;

v=1, 2, 3 or 4,

L₃ is

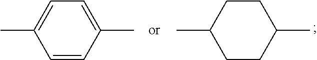

w=1;

R⁹ is H;

L₄ is —CH₂—;

z=1; r=0; L₂ is —NH—.

Aspect 30: The compound according to Aspect 17, wherein the Linker is selected from

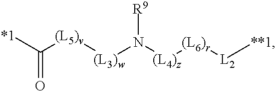

wherein

L₅ is —CH₂CH₂O—, —CH₂—, —CH═CH—, or —CH₂—O—CH₂—;

v=1, 2, 3 or 4, L₃ is —CH₂—, —C(O)—

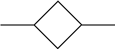

or —CH₂CH₂O—;

w=0, 1, 2 or 3; R⁹ is H or CH₃; L₄ is —CH₂CH₂O—, —CH₂—, —CH₂—O—CH₂—, —CH₂—CONN— or

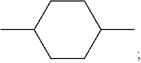

z=0, 1, 2, 3, 4, or 5; L₆ is —CH₂—,

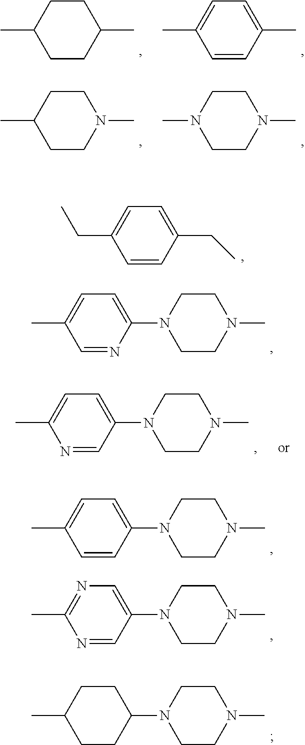

r=0, 1, 2, 3, 4, 5, or 6; L₂ is —NH—, —C(O)—, —O—,

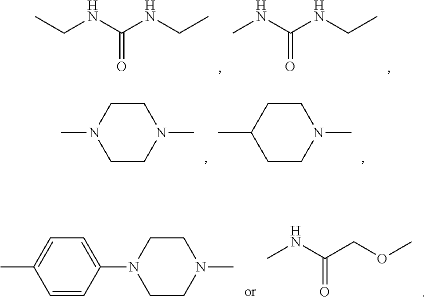

Aspect 31: the compound according to Aspect 30, wherein L₅ is —CH₂—: v=2; w=0; R⁹ is CH₃; L₄ is —CH₂—; z=1, 2, 3, or 4; L₆ is —CH₂—,

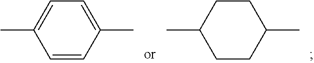

r=0, 1 or 2; L₂ is —NH—,

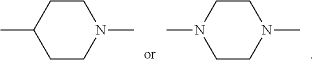

Aspect 32: the compound according to Aspect 30, wherein L₅ is —CH═CH—; v=1, L₃ is —CH₂—; w=0 or 1; R⁹ is H or CH₃; L₄ is —CH₂CH₂O— or —CH₂—; z=1, 2, 3, 4, 5 or 6; L₆ is —CH₂—; r=0, 1, 2; L₂ is —NH—, —CH₂—, or

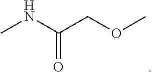

Aspect 33: the compound according to Aspect 30, wherein v=0;

L₃ is

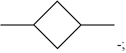

w=1;

R⁹ is CH₃;

L₄ is —CH₂—;

z=1 or 2;

L₆ is

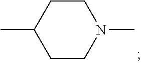

r=0 or 1;

L₂ is —NH—,

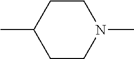

### or —C(O)—.

Aspect 34: the PROTAC compound according to Aspect 17, wherein the Linker is selected from

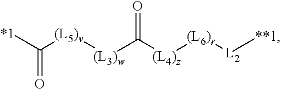

wherein

L₅ is-CH═CH—;

v=1, 2, 3 or 4;

L₃ is

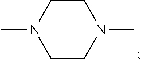

w=1;

L₄ is —CH₂—;

z=1, 2;

L₆ is —CH₂—;

r=0;

L₂ is —NH— or —CH₂—.

Aspect 35: the compound according to Aspect 17, wherein the Linker is selected from

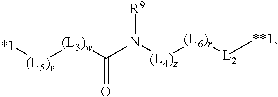

wherein

L₅ is

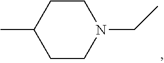

CH₂CH₂O—, —CH₂— or —CH₂—O—CH₂—;

v=1, 2, 3 or 4,

L₃ is —CH₂—,

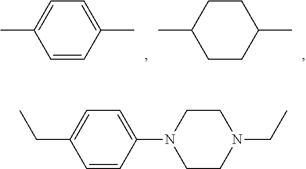

or —CH₂CH₂O—;

w=0, 1, 2 or 3;

R⁹ is H or CH₃;

L₄ is —CH₂—, —CH₂—O—CH₂—,

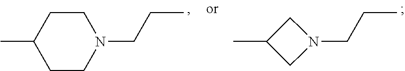

z=0, 1, 2, 3, or 4;

L₆ is —CH₂— or —OCH₂CH₂—;

r=0, 1, 2, 3, or 4;

L₂ is —NH—, —CH₂—, —O—,

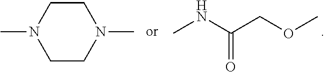

Aspect 36: the compound according to Aspect 17, wherein the Linker is selected from

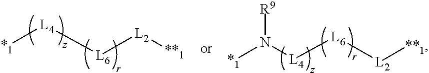

L₄ is

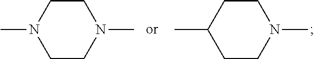

z=1;

L₆ is —CH₂—,

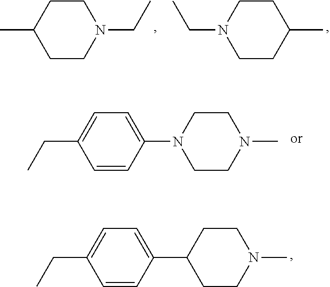

r=0, or 1;

L₂ is

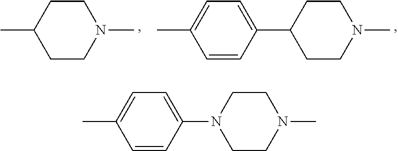

### or —C(O)—;

R⁹ is H or CH₃.

Aspect 37: The compound according to Aspect 1, wherein the Linker is selected from

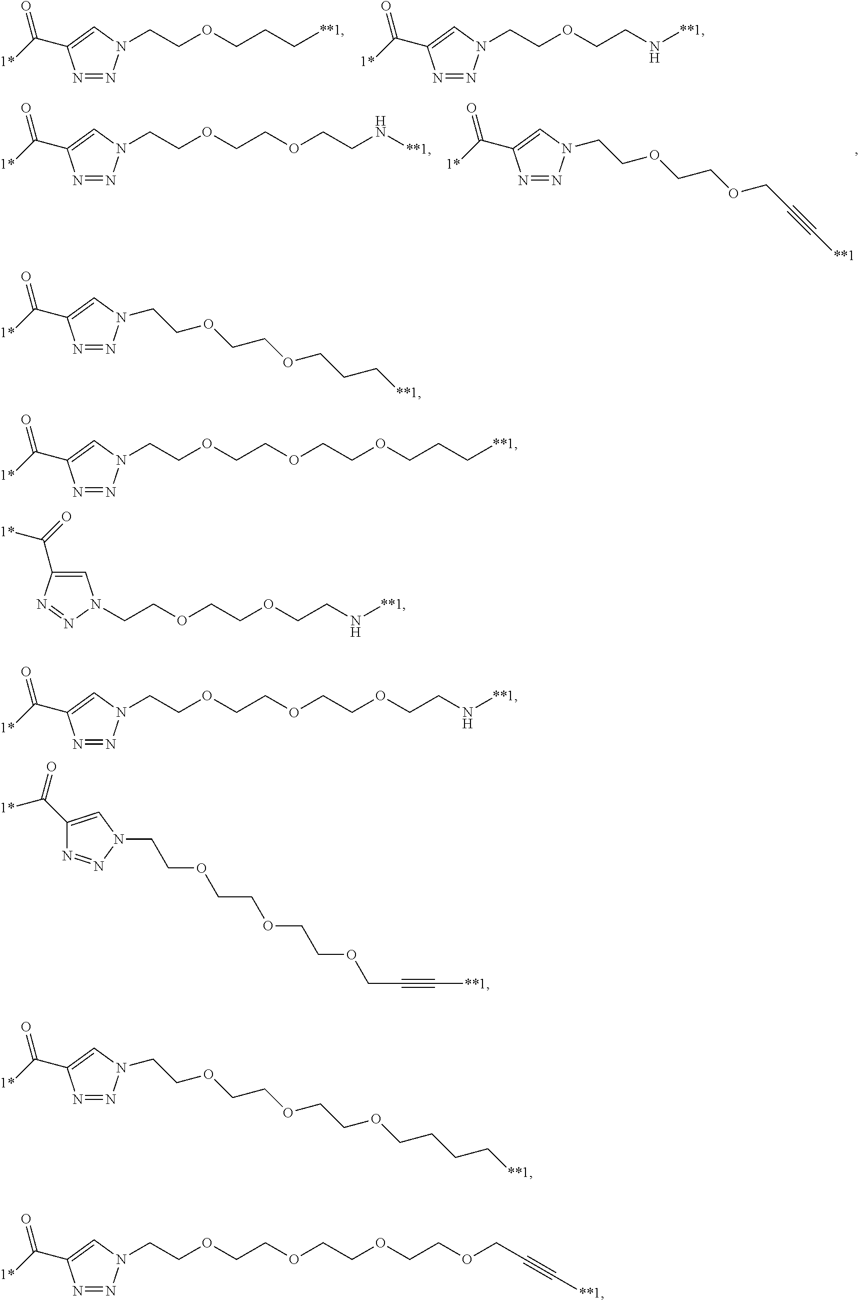

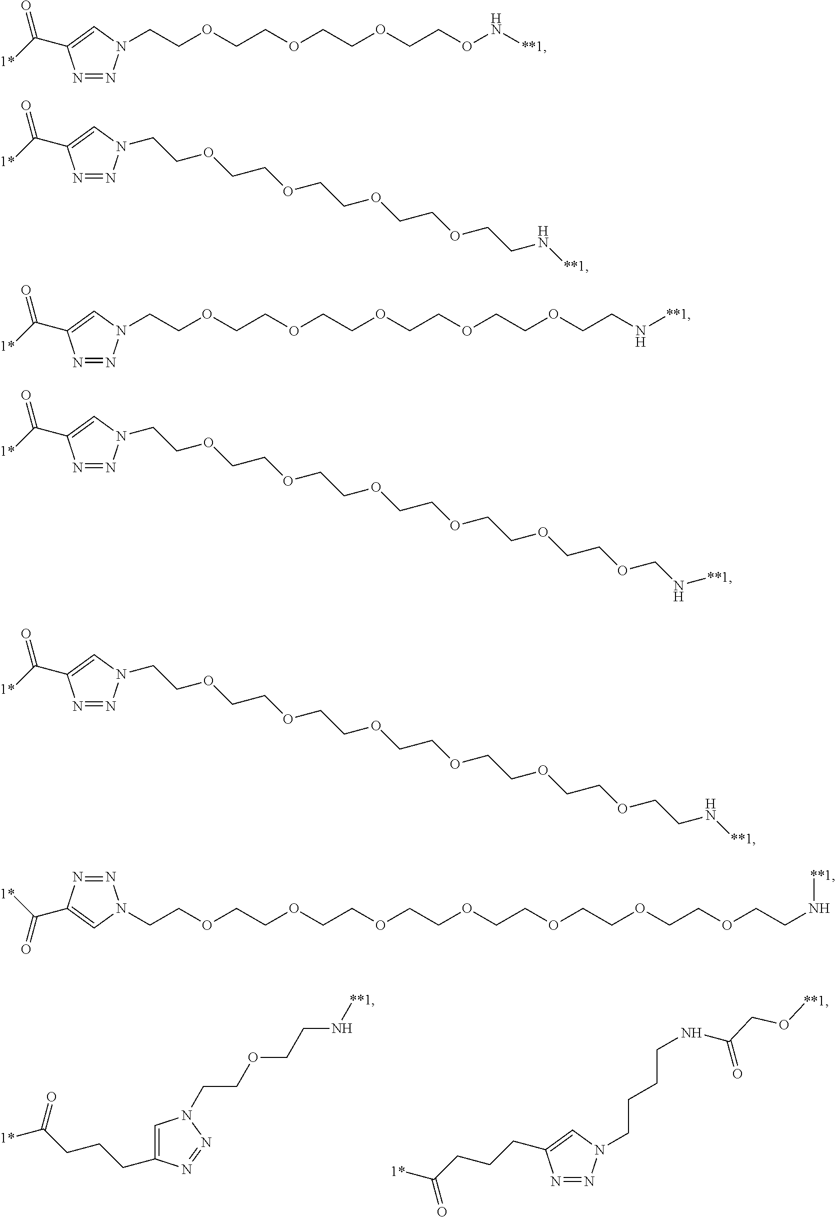

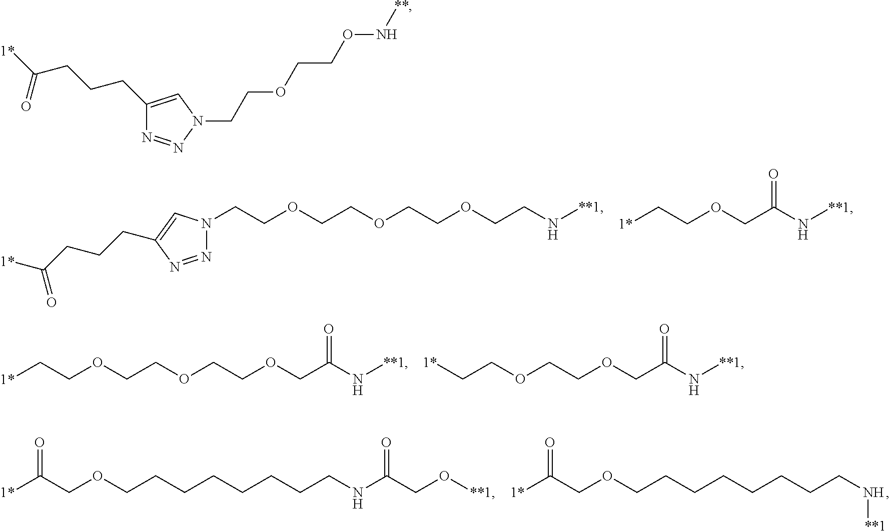

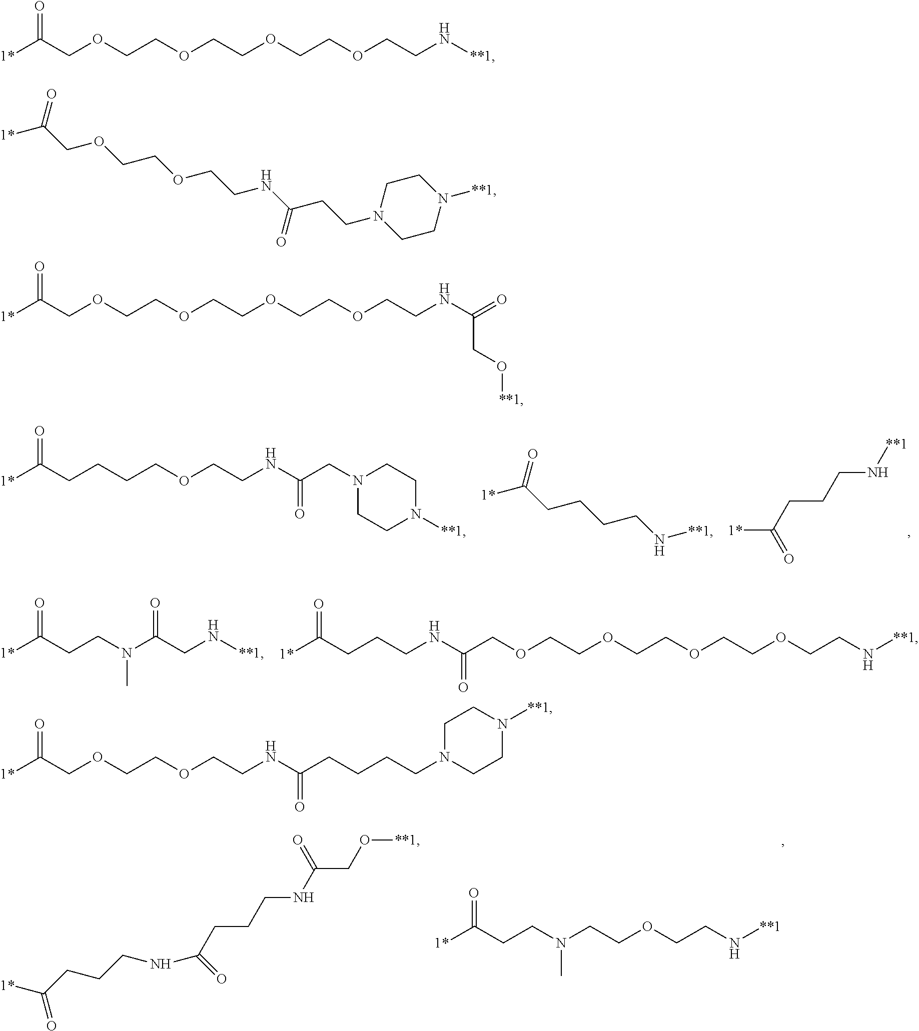

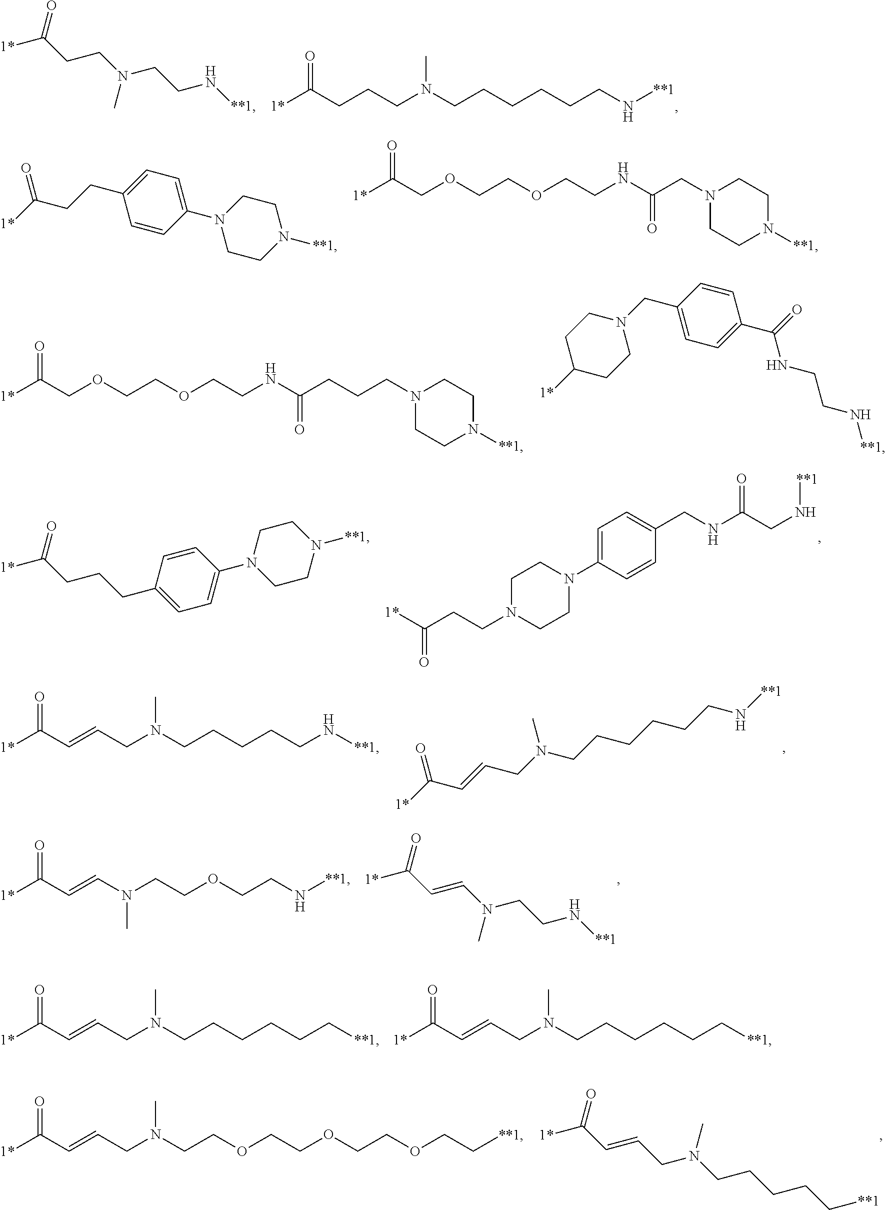

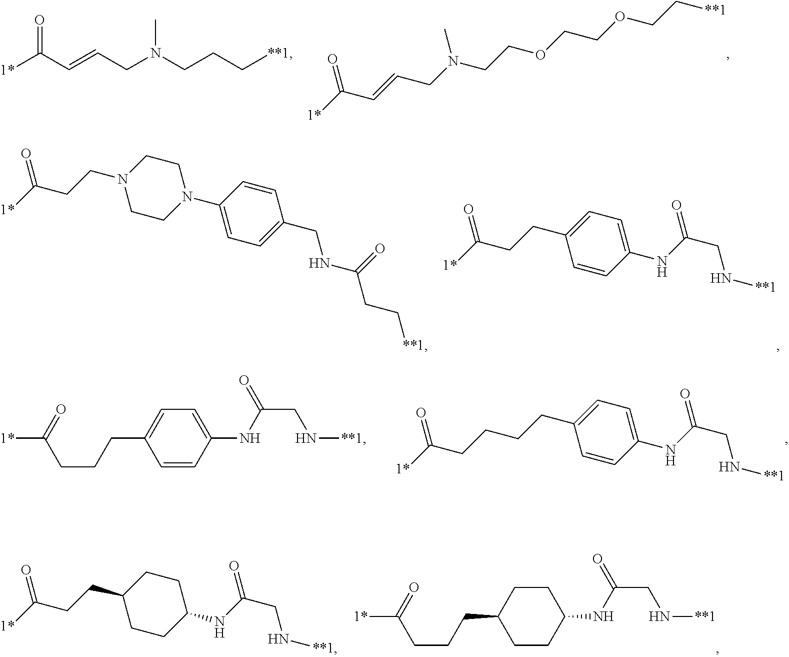

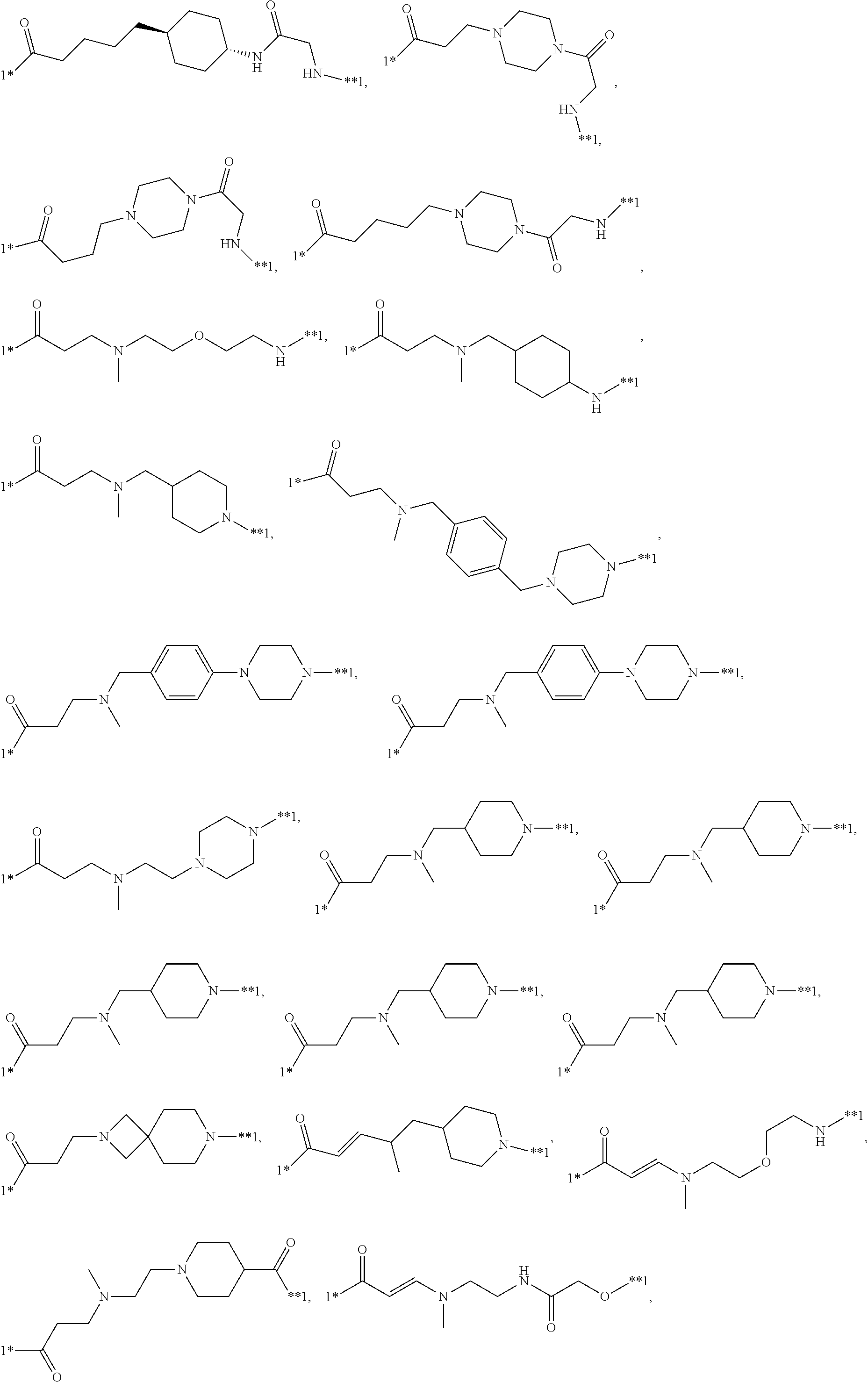

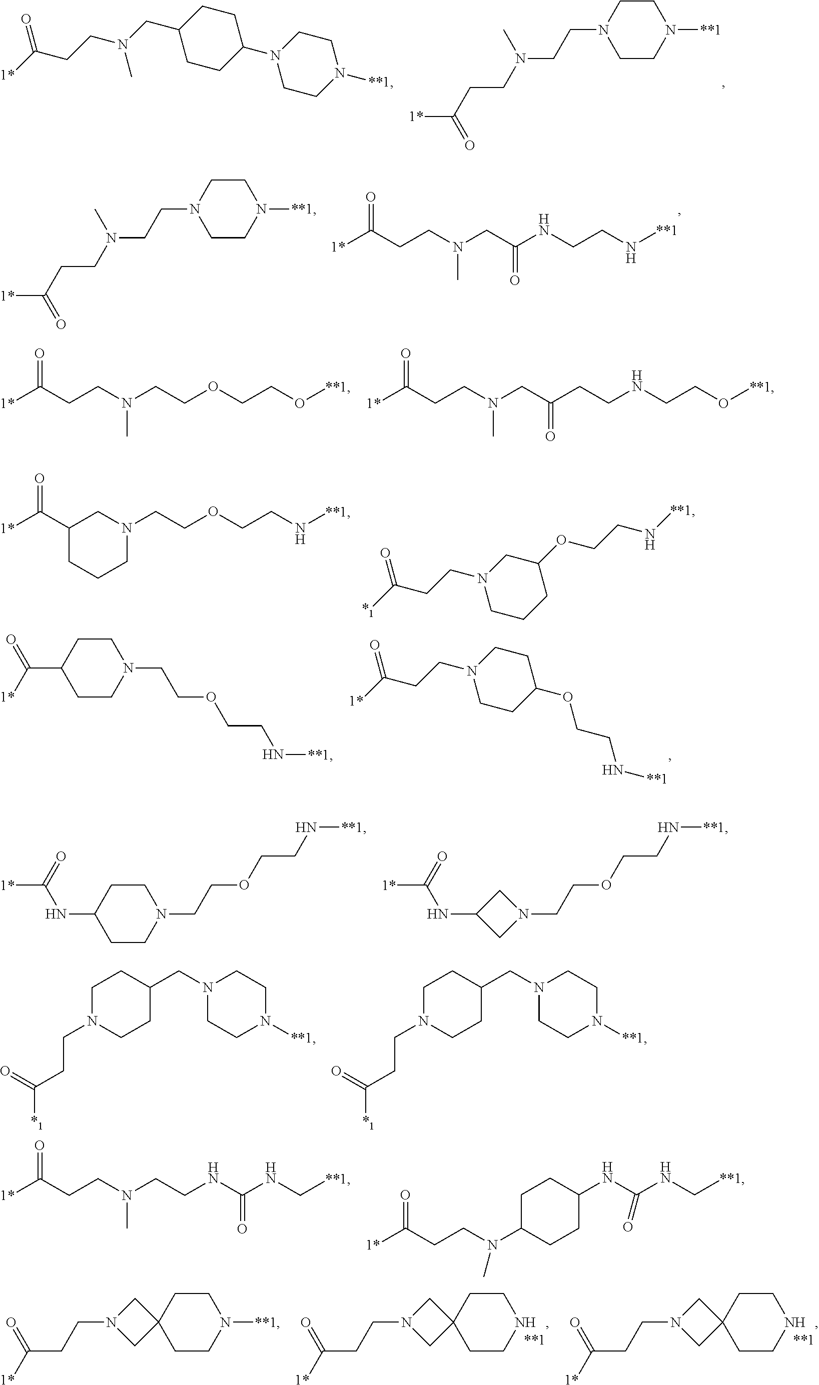

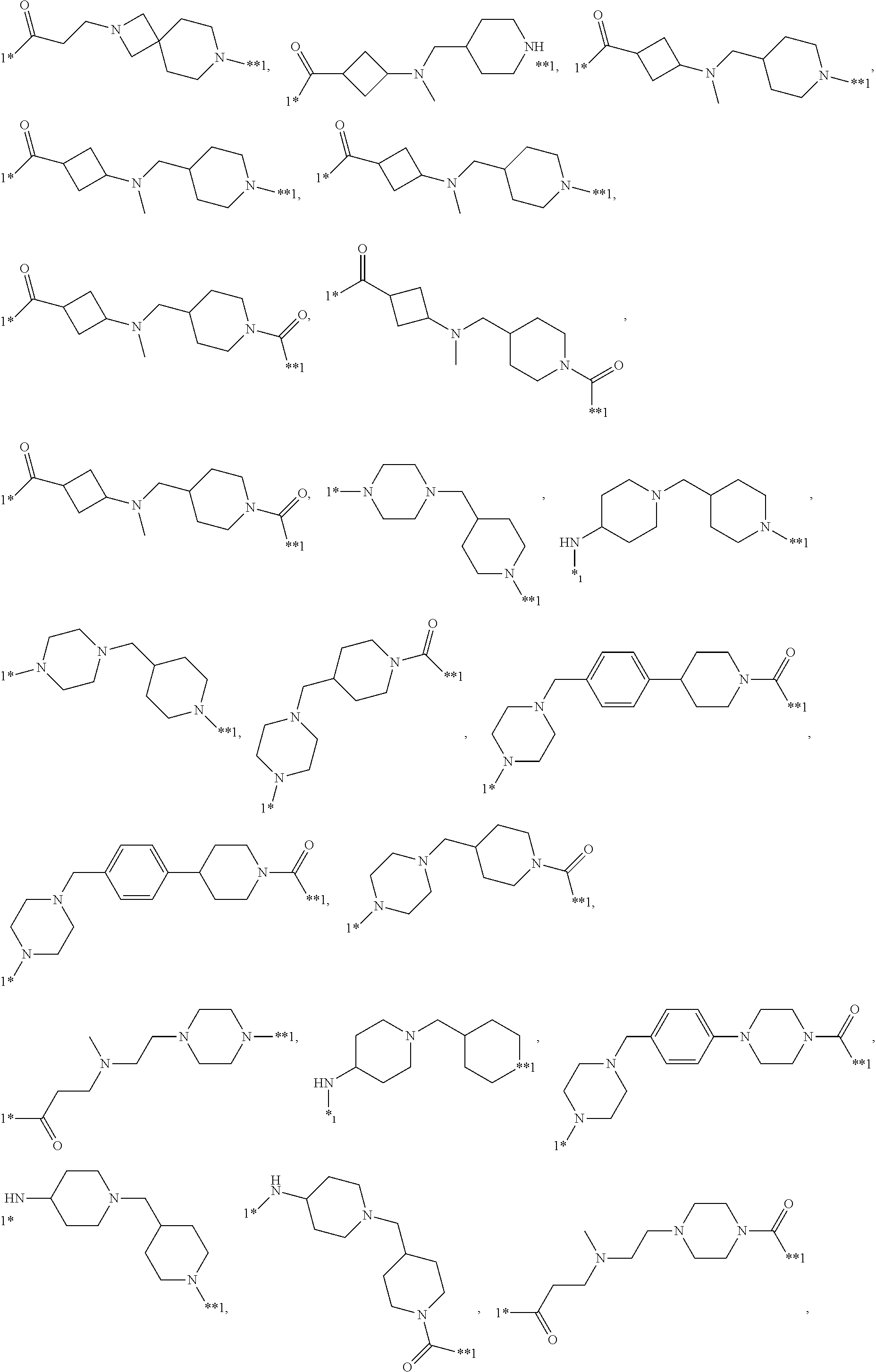

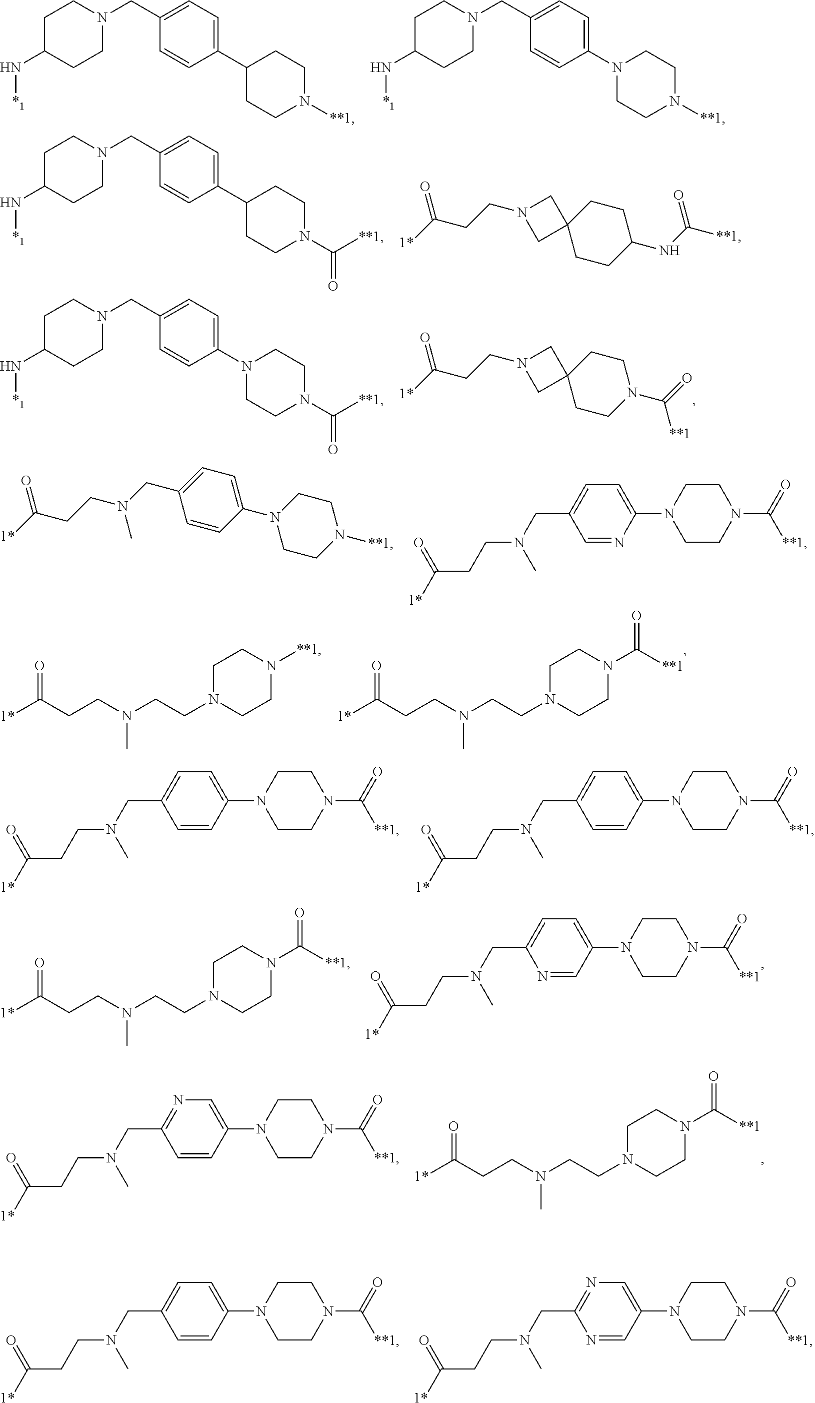

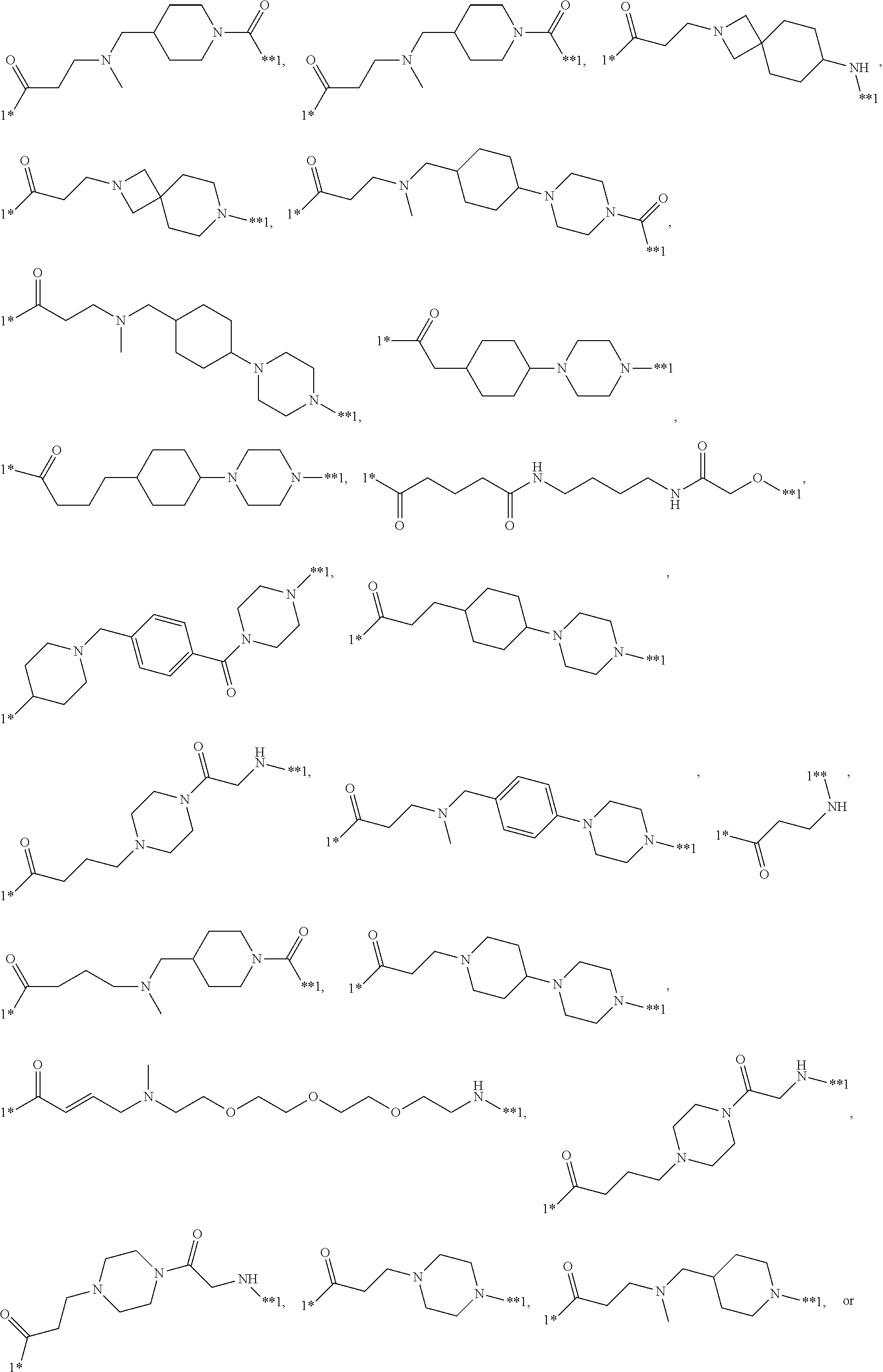

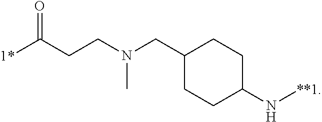

Aspect 38: The compound according to Aspect 1, wherein the compound is selected from

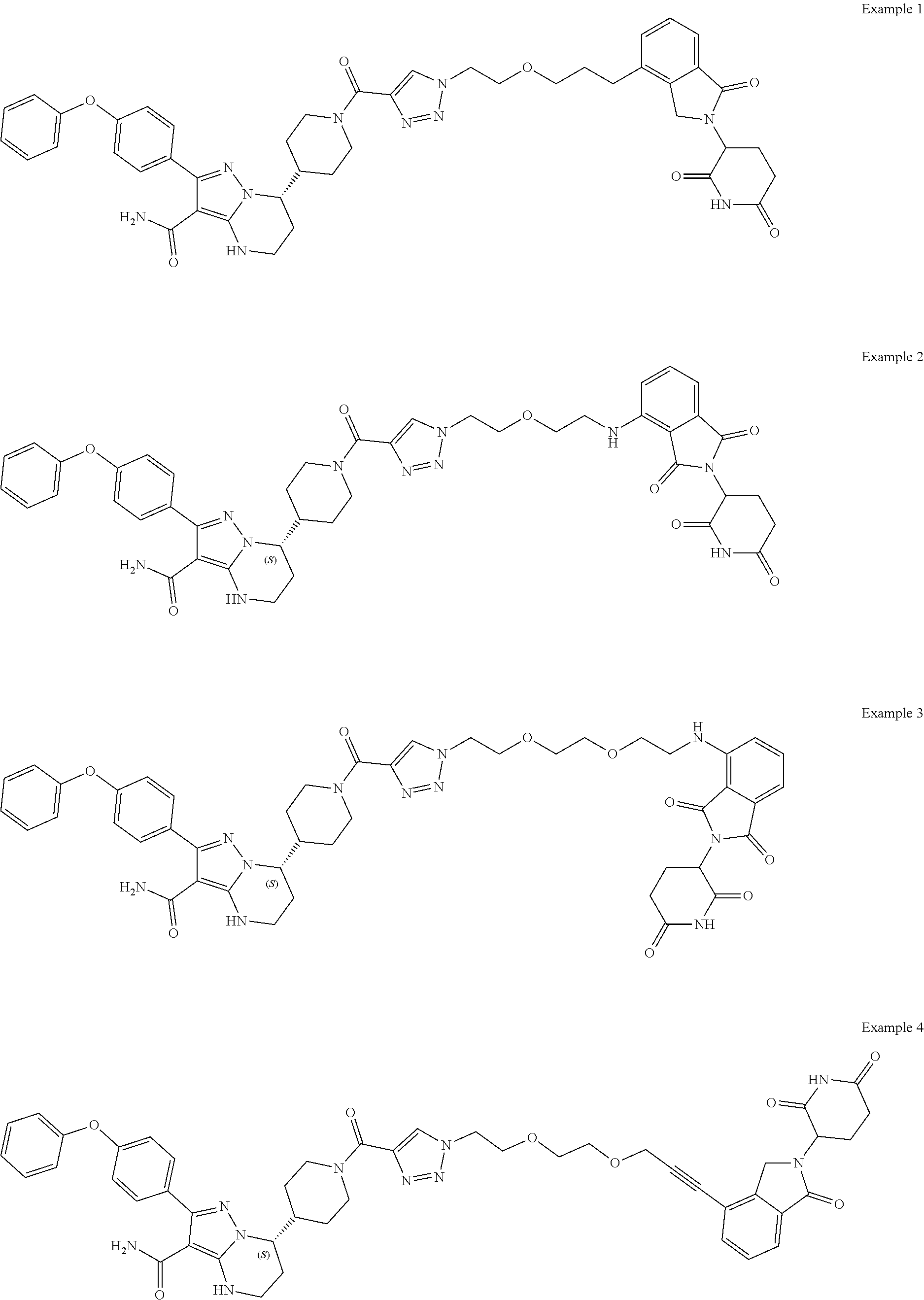

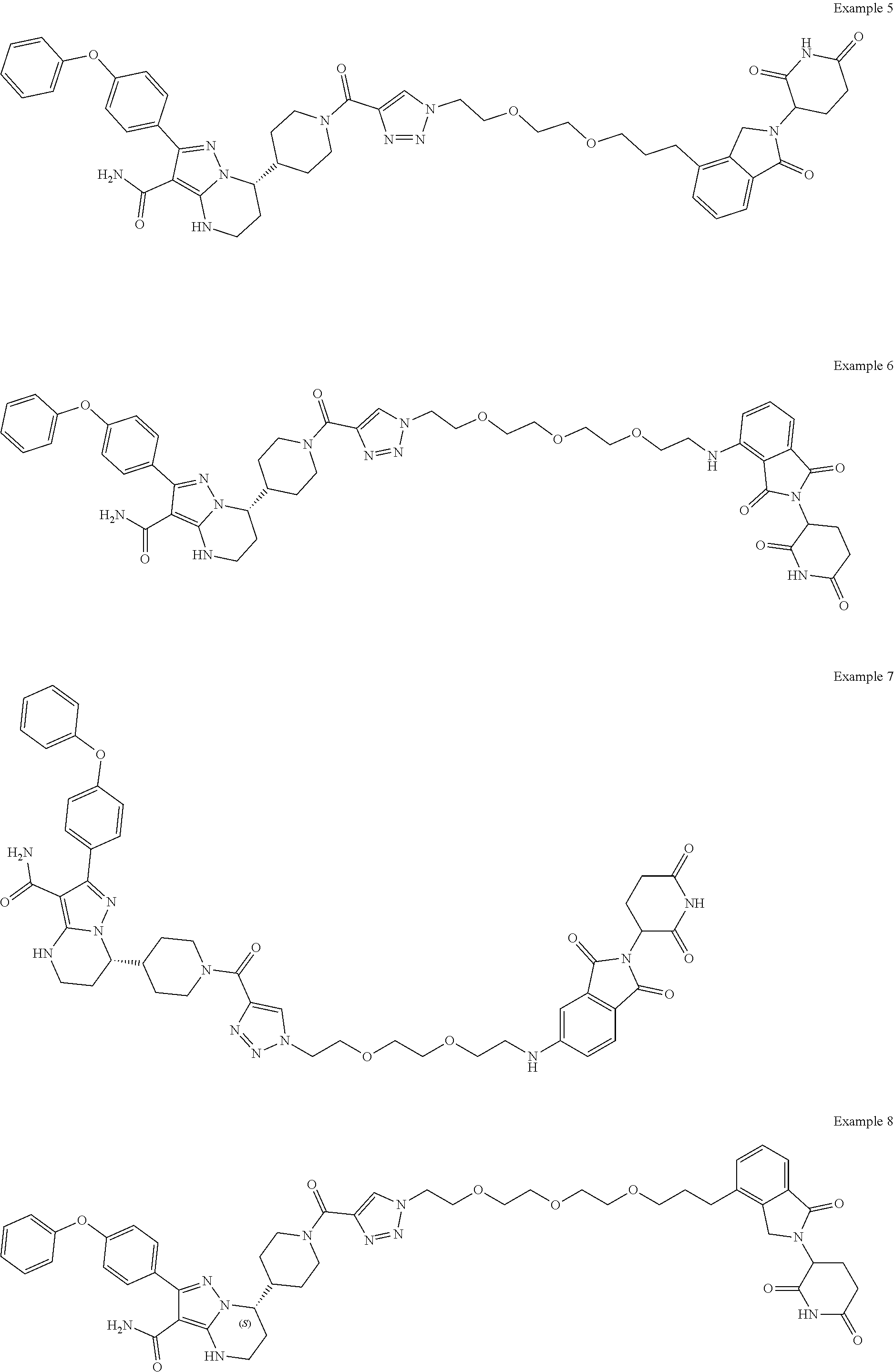

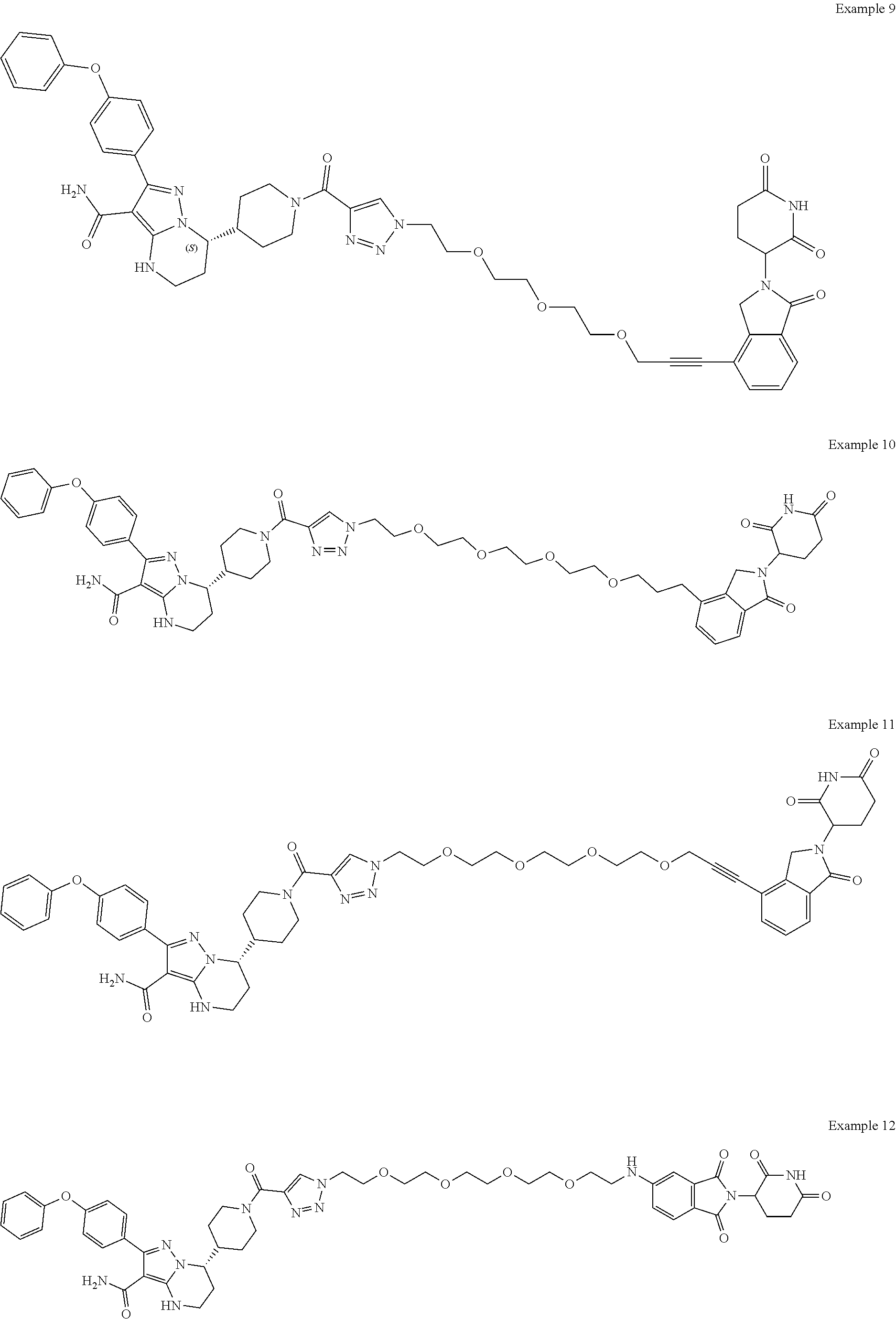

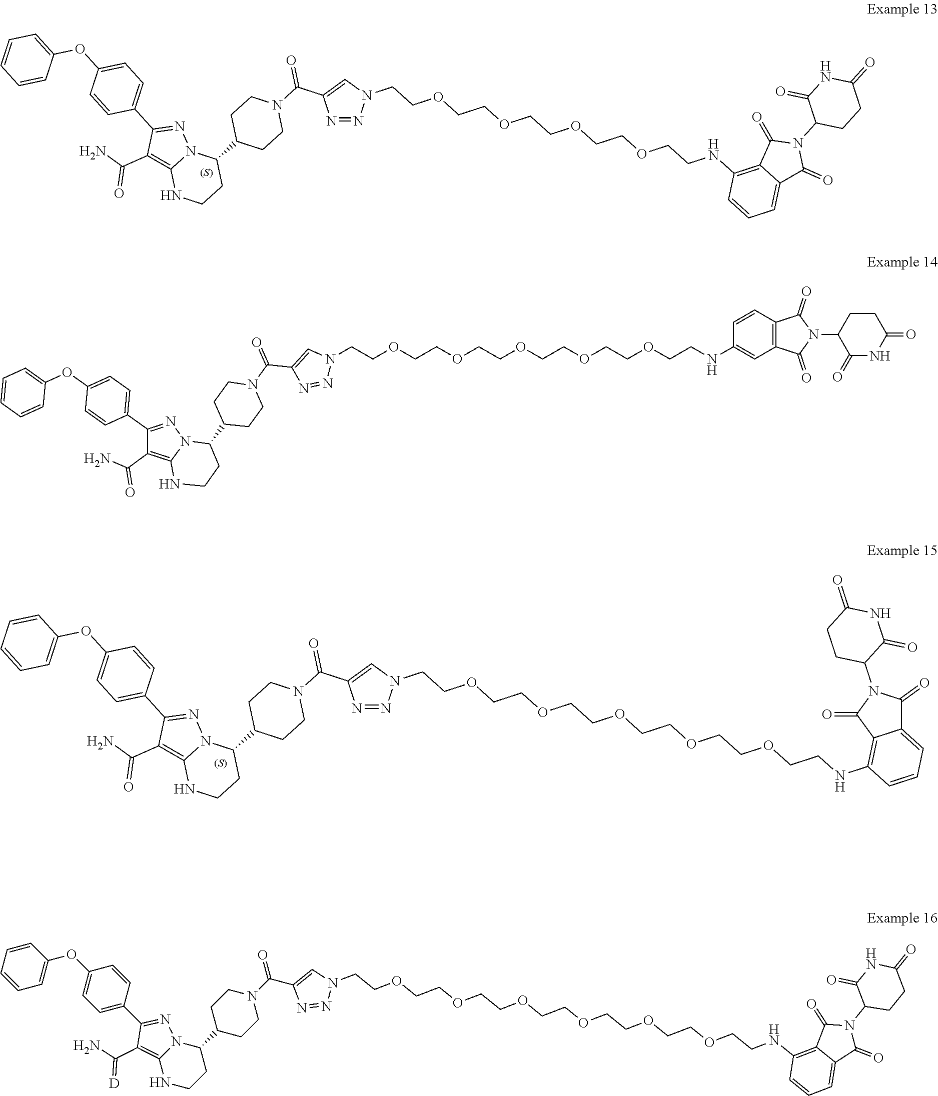

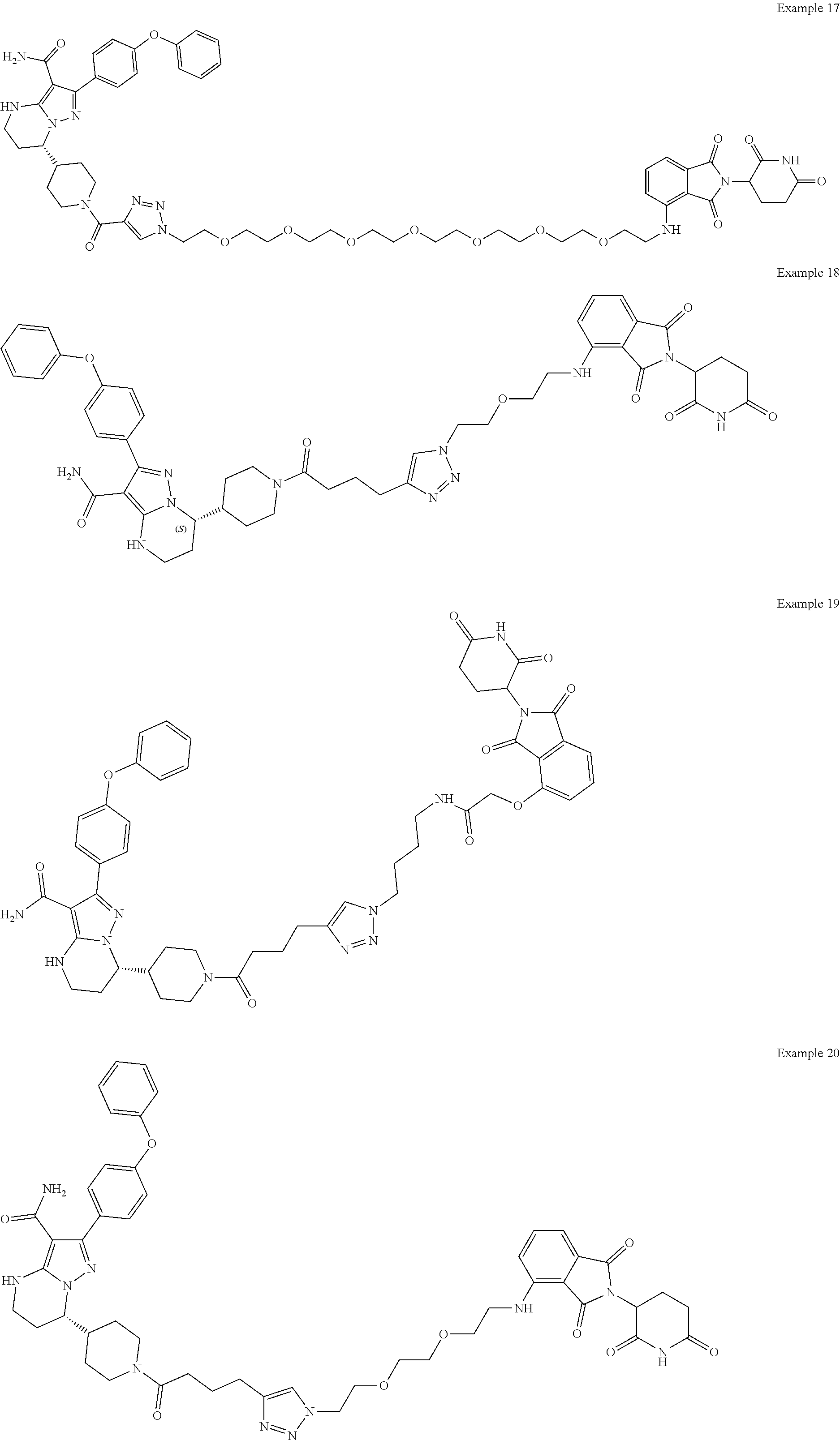

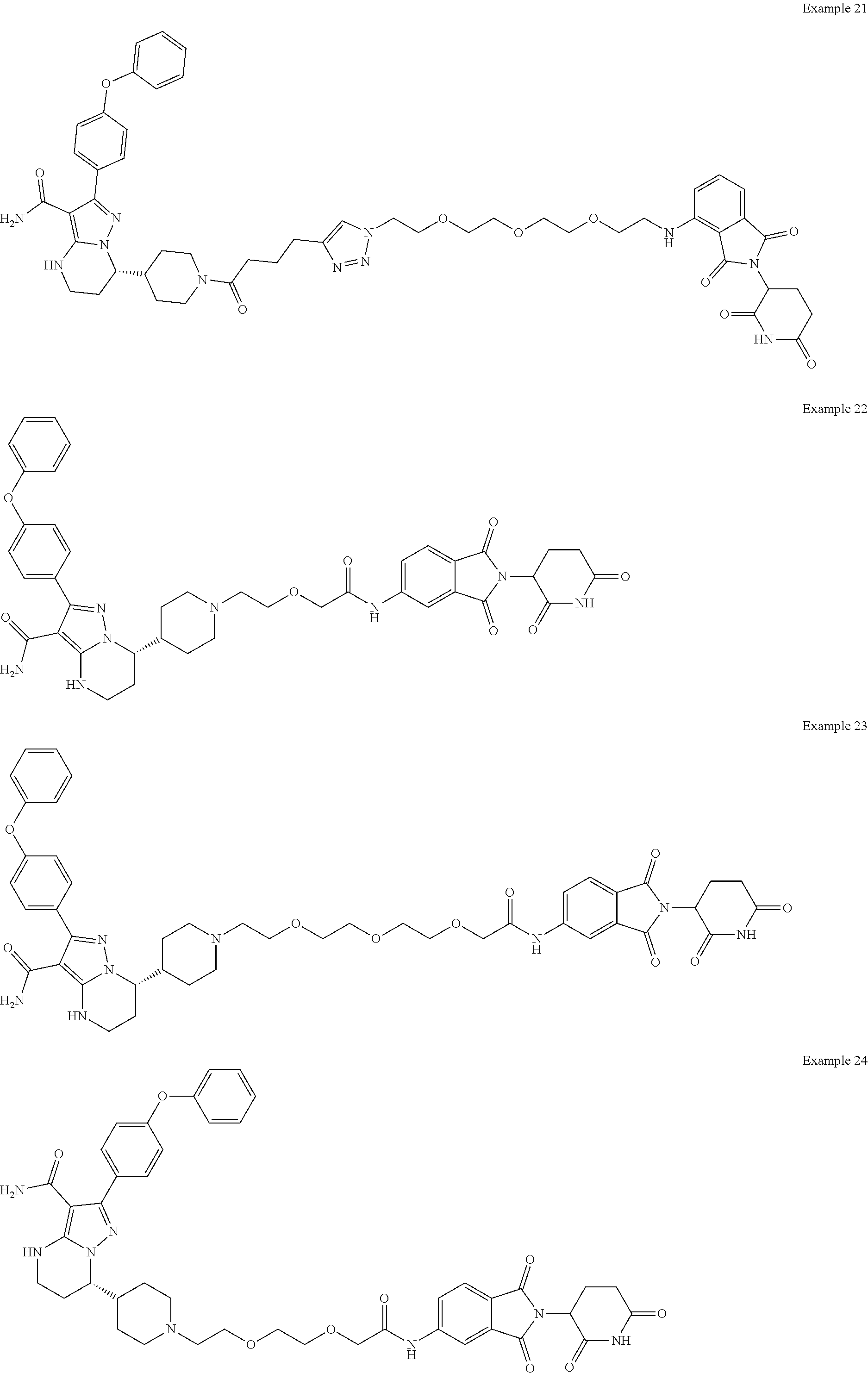 [SMILES: NC(=O)c1c(-c2ccc(Oc3ccccc3)cc2)nn2c1NCC[C@H]2C1CCN(C(=O)CCCc2cn(CCOCCOCCOCCNc3cccc4c3C(=O)N(C3CCC(=O)NC3=O)C4=O)nn2)CC1 ; NC(=O)c1c(-c2ccc(Oc3ccccc3)cc2)nn2c1NCC[C@H]2C1CCN(CCOCC(=O)Nc2ccc3c(c2)C(=O)N(C2CCC(=O)NC2=O)C3=O)CC1 ; NC(=O)c1c(-c2ccc(Oc3ccccc3)cc2)nn2c1NCC[C@H]2C1CCN(CCOCCOCCOCC(=O)Nc2ccc3c(c2)C(=O)N(C2CCC(=O)NC2=O)C3=O)CC1 ; NC(=O)c1c(-c2ccc(Oc3ccccc3)cc2)nn2c1NCC[C@H]2C1CCN(CCOCCOCC(=O)Nc2ccc3c(c2)C(=O)N(C2CCC(=O)NC2=O)C3=O)CC1]

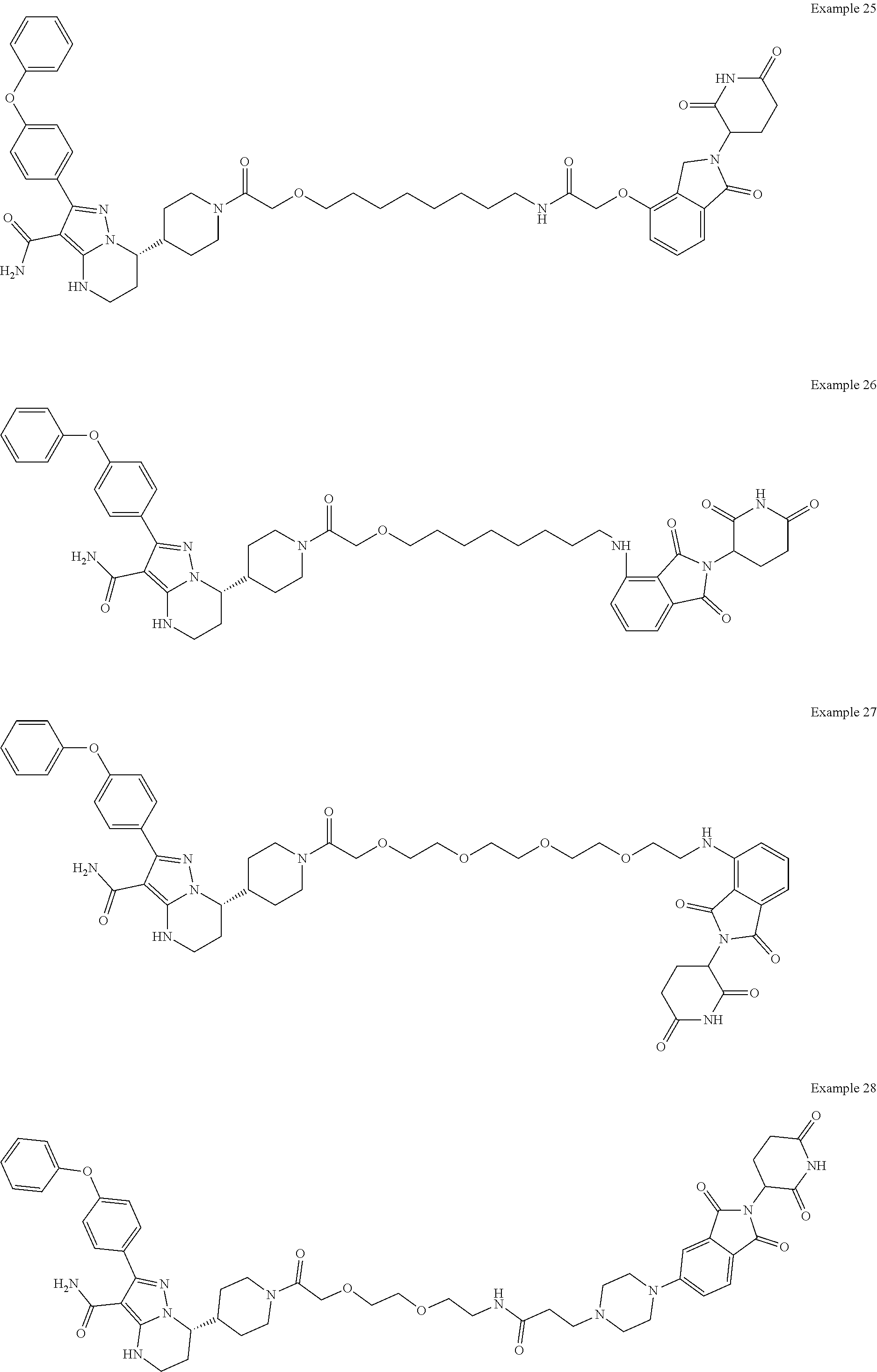 [SMILES: NC(=O)c1c(-c2ccc(Oc3ccccc3)cc2)nn2c1NCC[C@H]2C1CCN(C(=O)COCCCCCCCCNC(=O)COc2cccc3c2CN(C2CCC(=O)NC2=O)C3=O)CC1 ; NC(=O)c1c(-c2ccc(Oc3ccccc3)cc2)nn2c1NCC[C@H]2C1CCN(C(=O)COCCCCCCCCNc2cccc3c2C(=O)N(C2CCC(=O)NC2=O)C3=O)CC1 ; NC(=O)c1c(-c2ccc(Oc3ccccc3)cc2)nn2c1NCC[C@H]2C1CCN(C(=O)COCCOCCOCCOCCNc2cccc3c2C(=O)N(C2CCC(=O)NC2=O)C3=O)CC1 ; NC(=O)c1c(-c2ccc(Oc3ccccc3)cc2)nn2c1NCC[C@H]2C1CCN(C(=O)COCCOCCNC(=O)CCN2CCN(c3ccc4c(c3)C(=O)N(C3CCC(=O)NC3=O)C4=O)CC2)CC1]

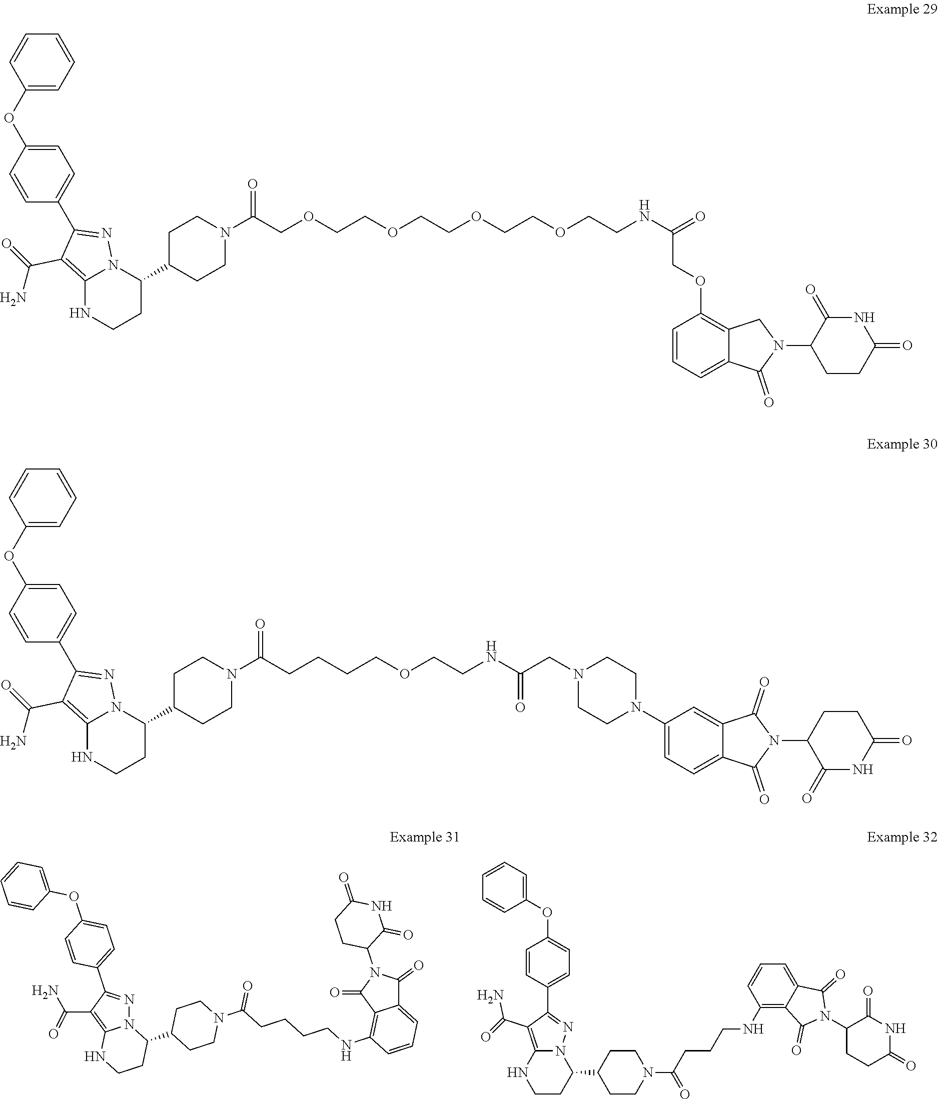 [SMILES: NC(=O)c1c(-c2ccc(Oc3ccccc3)cc2)nn2c1NCC[C@H]2C1CCN(C(=O)COCCOCCOCCOCCNC(=O)COc2cccc3c2CN(C2CCC(=O)NC2=O)C3=O)CC1 ; NC(=O)c1c(-c2ccc(Oc3ccccc3)cc2)nn2c1NCC[C@H]2C1CCN(C(=O)CCCCOCCNC(=O)CN2CCN(c3ccc4c(c3)C(=O)N(C3CCC(=O)NC3=O)C4=O)CC2)CC1 ; NC(=O)c1c(-c2ccc(Oc3ccccc3)cc2)nn2c1NCC[C@H]2C1CCN(C(=O)CCCCNc2cccc3c2C(=O)N(C2CCC(=O)NC2=O)C3=O)CC1 ; NC(=O)c1c(-c2ccc(Oc3ccccc3)cc2)nn2c1NCC[C@H]2C1CCN(C(=O)CCCNc2cccc3c2C(=O)N(C2CCC(=O)NC2=O)C3=O)CC1]

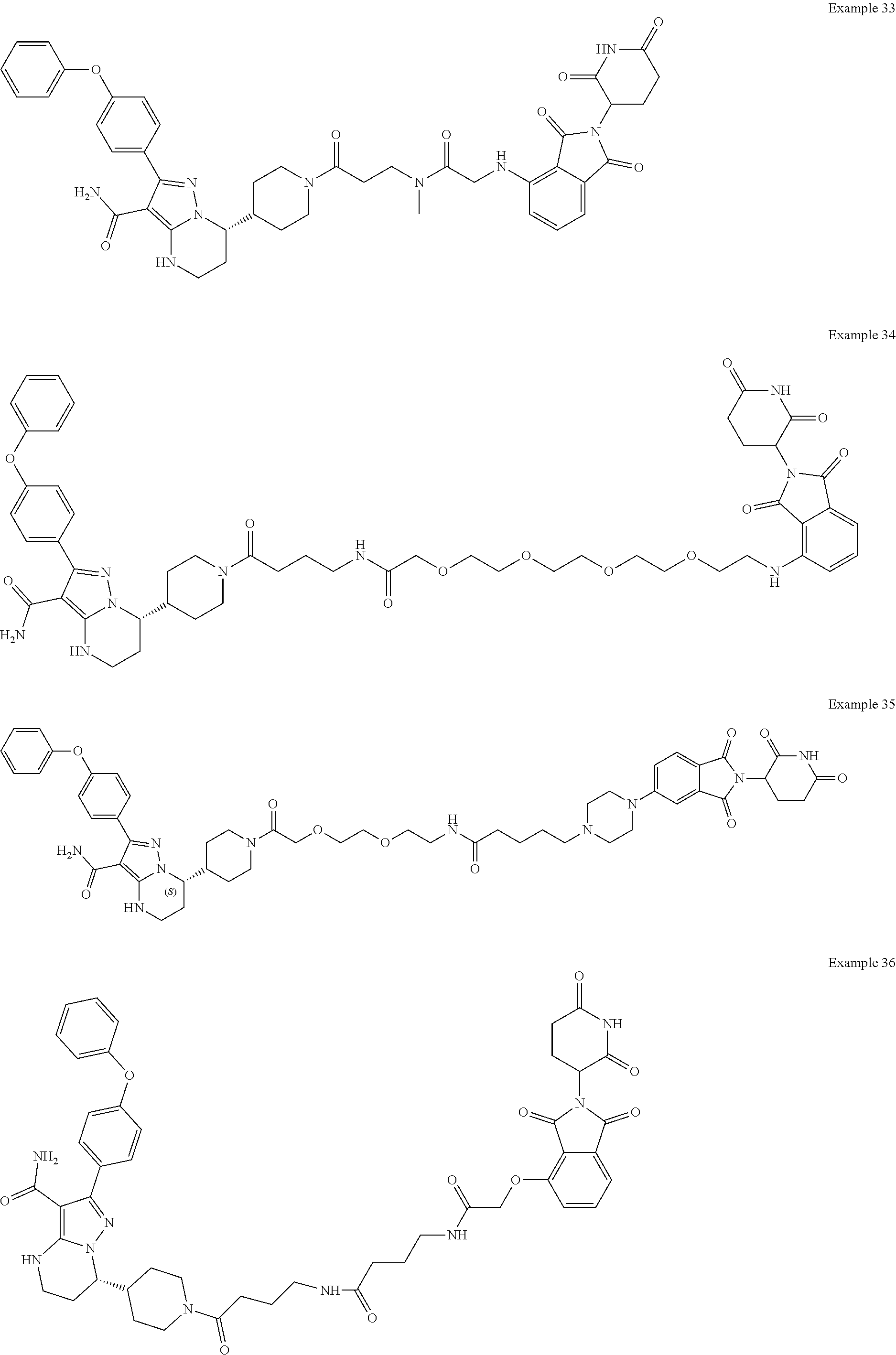

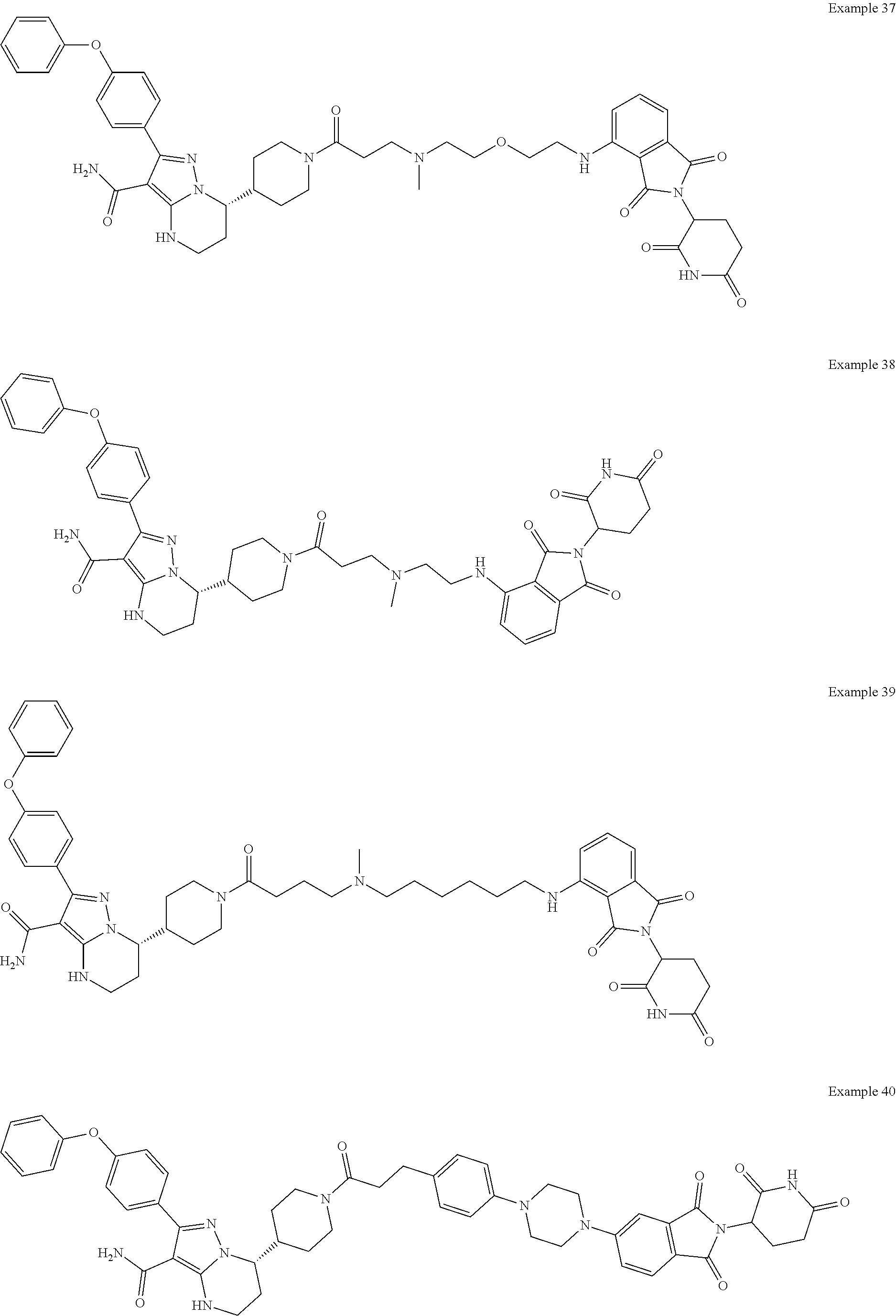 [SMILES: CN(CCOCCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1 ; CN(CCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1 ; CN(CCCCCCNc1cccc2c1C(=O)N(C1CCC(=O)NC1=O)C2=O)CCCC(=O)N1CCC([C@@H]2CCNc3c(C(N)=O)c(-c4ccc(Oc5ccccc5)cc4)nn32)CC1 ; NC(=O)c1c(-c2ccc(Oc3ccccc3)cc2)nn2c1NCC[C@H]2C1CCN(C(=O)CCc2ccc(N3CCN(c4ccc5c(c4)C(=O)N(C4CCC(=O)NC4=O)C5=O)CC3)cc2)CC1]

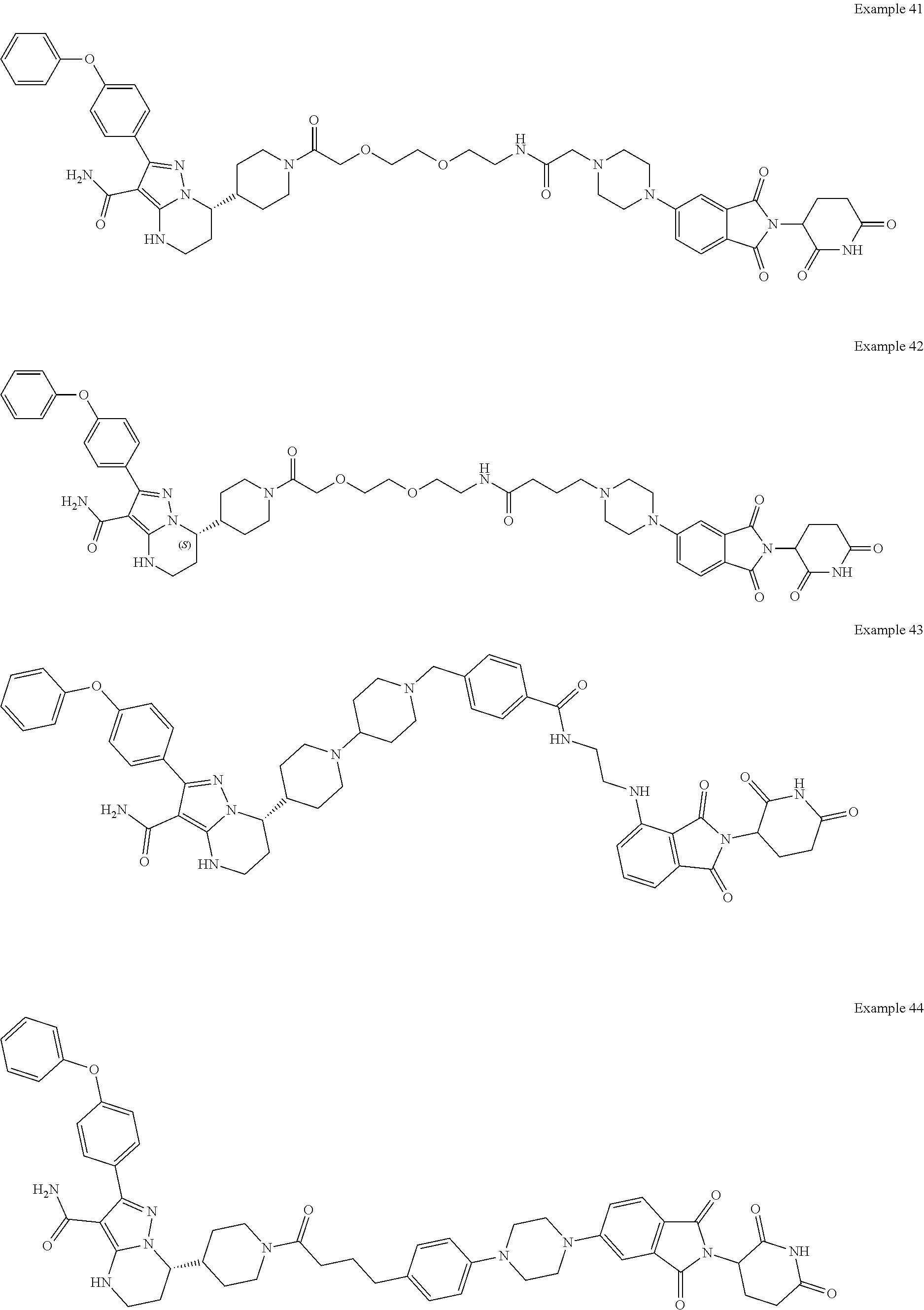

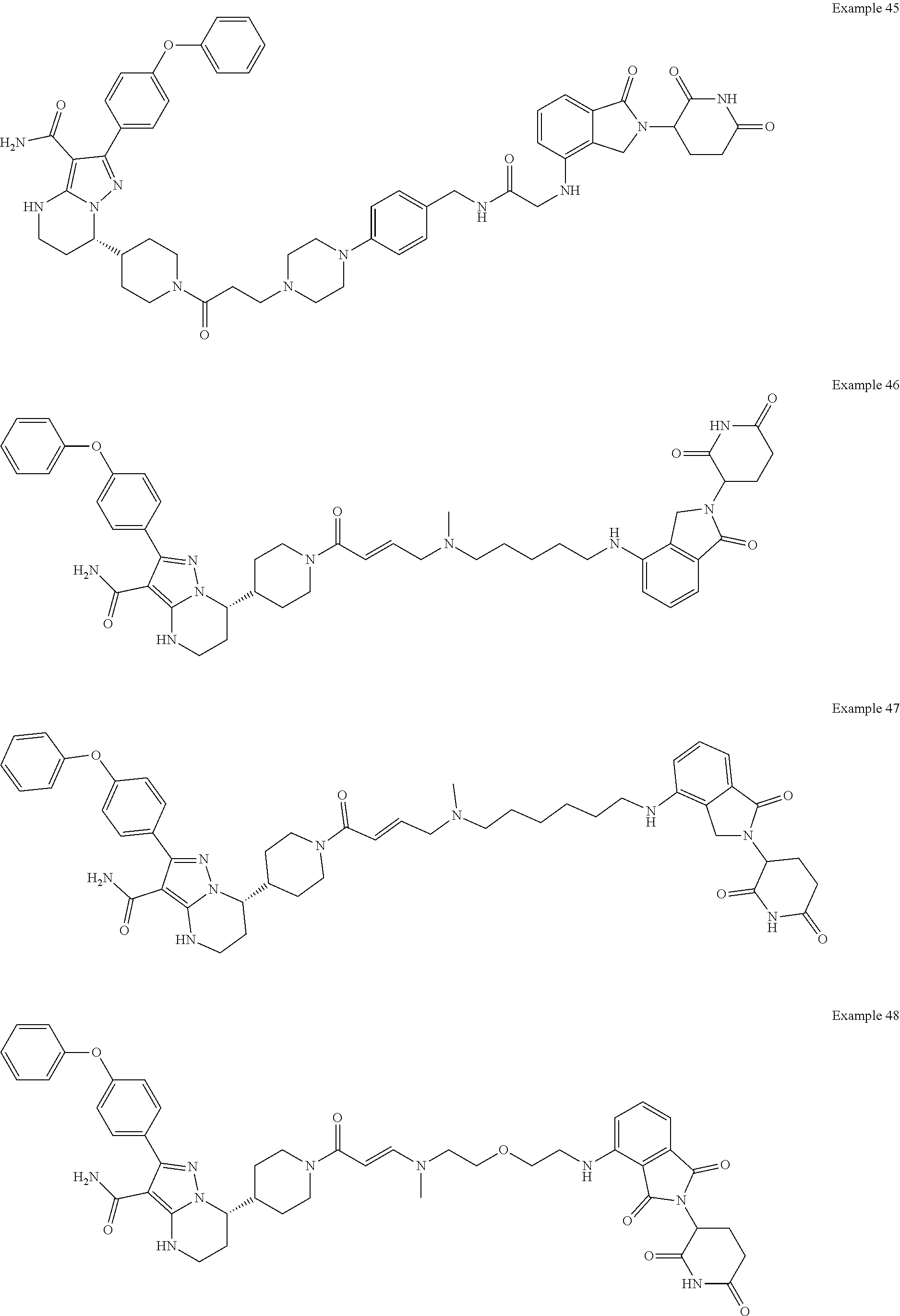 [SMILES: NC(=O)c1c(-c2ccc(Oc3ccccc3)cc2)nn2c1NCC[C@H]2C1CCN(C(=O)CCN2CCN(c3ccc(CNC(=O)CNc4cccc5c4CN(C4CCC(=O)NC4=O)C5=O)cc3)CC2)CC1 ; CN(C/C=C/C(=O)N1CCC([C@@H]2CCNc3c(C(N)=O)c(-c4ccc(Oc5ccccc5)cc4)nn32)CC1)CCCCCNc1cccc2c1CN(C1CCC(=O)NC1=O)C2=O ; CN(C/C=C/C(=O)N1CCC([C@@H]2CCNc3c(C(N)=O)c(-c4ccc(Oc5ccccc5)cc4)nn32)CC1)CCCCCCNc1cccc2c1CN(C1CCC(=O)NC1=O)C2=O ; CN(/C=C/C(=O)N1CCC([C@@H]2CCNc3c(C(N)=O)c(-c4ccc(Oc5ccccc5)cc4)nn32)CC1)CCOCCNc1cccc2c1C(=O)N(C1CCC(=O)NC1=O)C2=O]

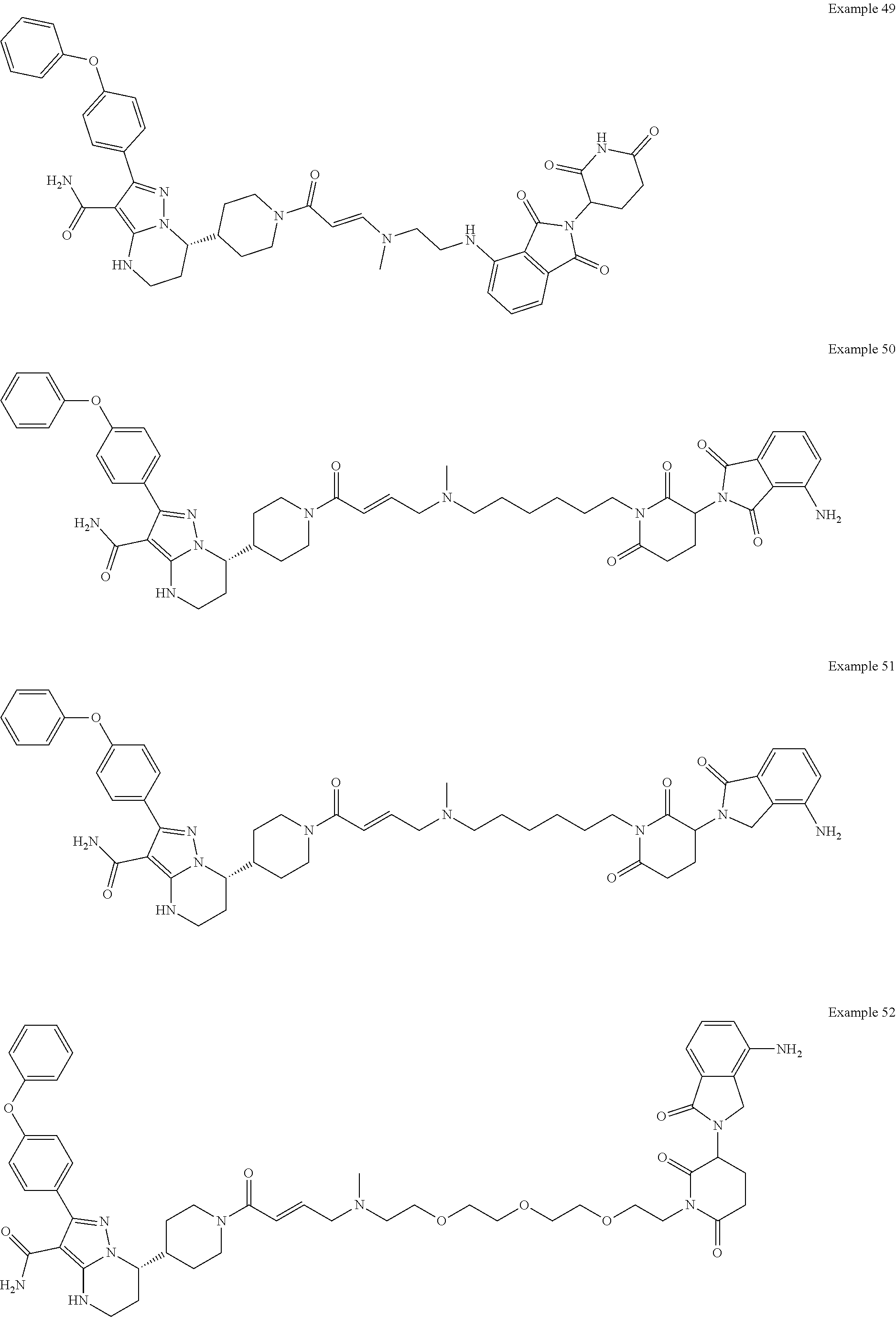 [SMILES: CN(/C=C/C(=O)N1CCC([C@@H]2CCNc3c(C(N)=O)c(-c4ccc(Oc5ccccc5)cc4)nn32)CC1)CCNc1cccc2c1C(=O)N(C1CCC(=O)NC1=O)C2=O ; CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2C(=O)c3cccc(N)c3C2=O)C1=O ; CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2Cc3c(N)cccc3C2=O)C1=O ; CN(C/C=C/C(=O)N1CCC([C@@H]2CCNc3c(C(N)=O)c(-c4ccc(Oc5ccccc5)cc4)nn32)CC1)CCOCCOCCOCCN1C(=O)CCC(N2Cc3c(N)cccc3C2=O)C1=O]

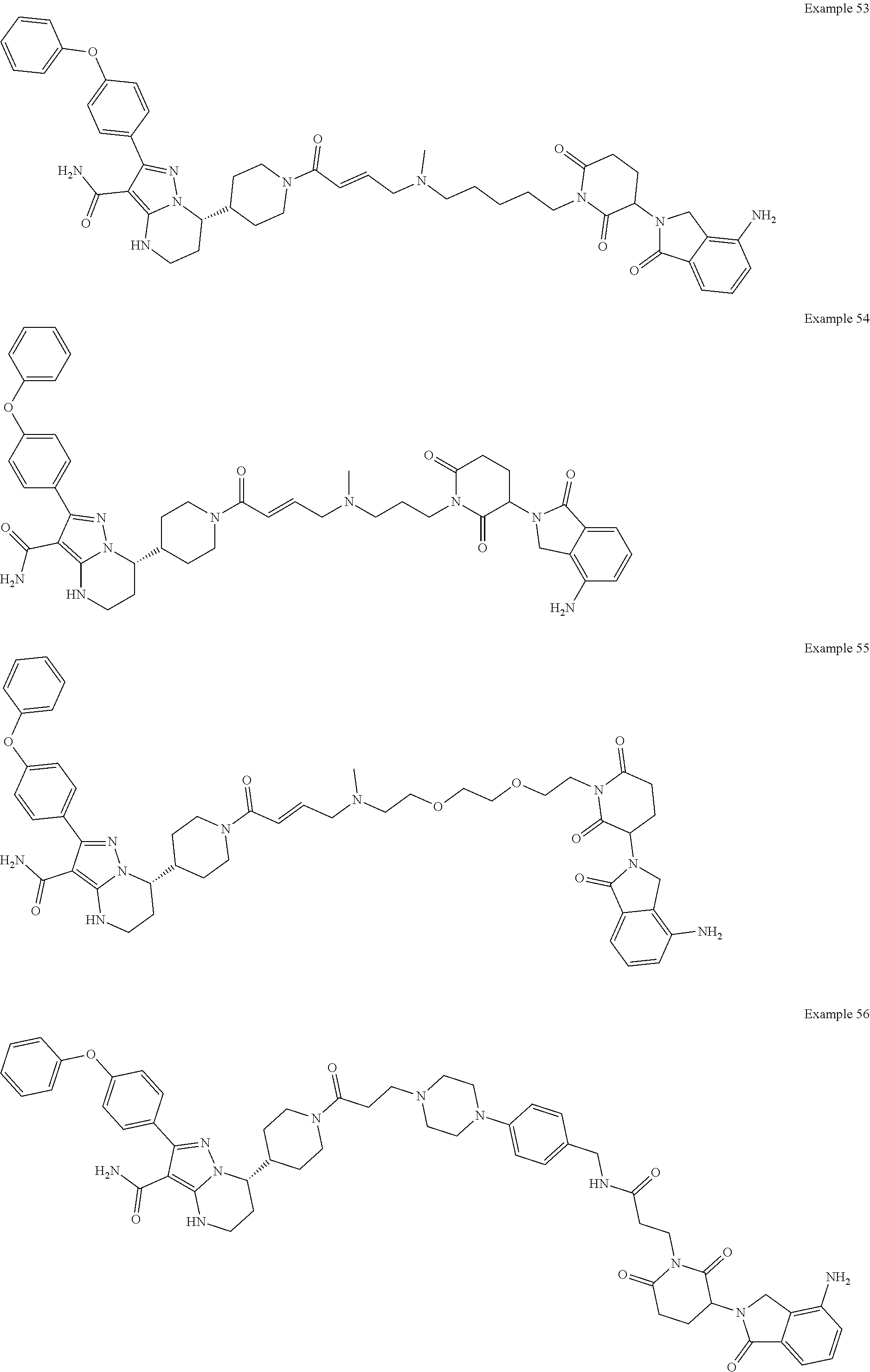 [SMILES: CN(C/C=C/C(=O)N1CCC([C@@H]2CCNc3c(C(N)=O)c(-c4ccc(Oc5ccccc5)cc4)nn32)CC1)CCCCCN1C(=O)CCC(N2Cc3c(N)cccc3C2=O)C1=O ; CN(C/C=C/C(=O)N1CCC([C@@H]2CCNc3c(C(N)=O)c(-c4ccc(Oc5ccccc5)cc4)nn32)CC1)CCCN1C(=O)CCC(N2Cc3c(N)cccc3C2=O)C1=O ; CN(C/C=C/C(=O)N1CCC([C@@H]2CCNc3c(C(N)=O)c(-c4ccc(Oc5ccccc5)cc4)nn32)CC1)CCOCCOCCN1C(=O)CCC(N2Cc3c(N)cccc3C2=O)C1=O ; NC(=O)c1c(-c2ccc(Oc3ccccc3)cc2)nn2c1NCC[C@H]2C1CCN(C(=O)CCN2CCN(c3ccc(CNC(=O)CCN4C(=O)CCC(N5Cc6c(N)cccc6C5=O)C4=O)cc3)CC2)CC1]

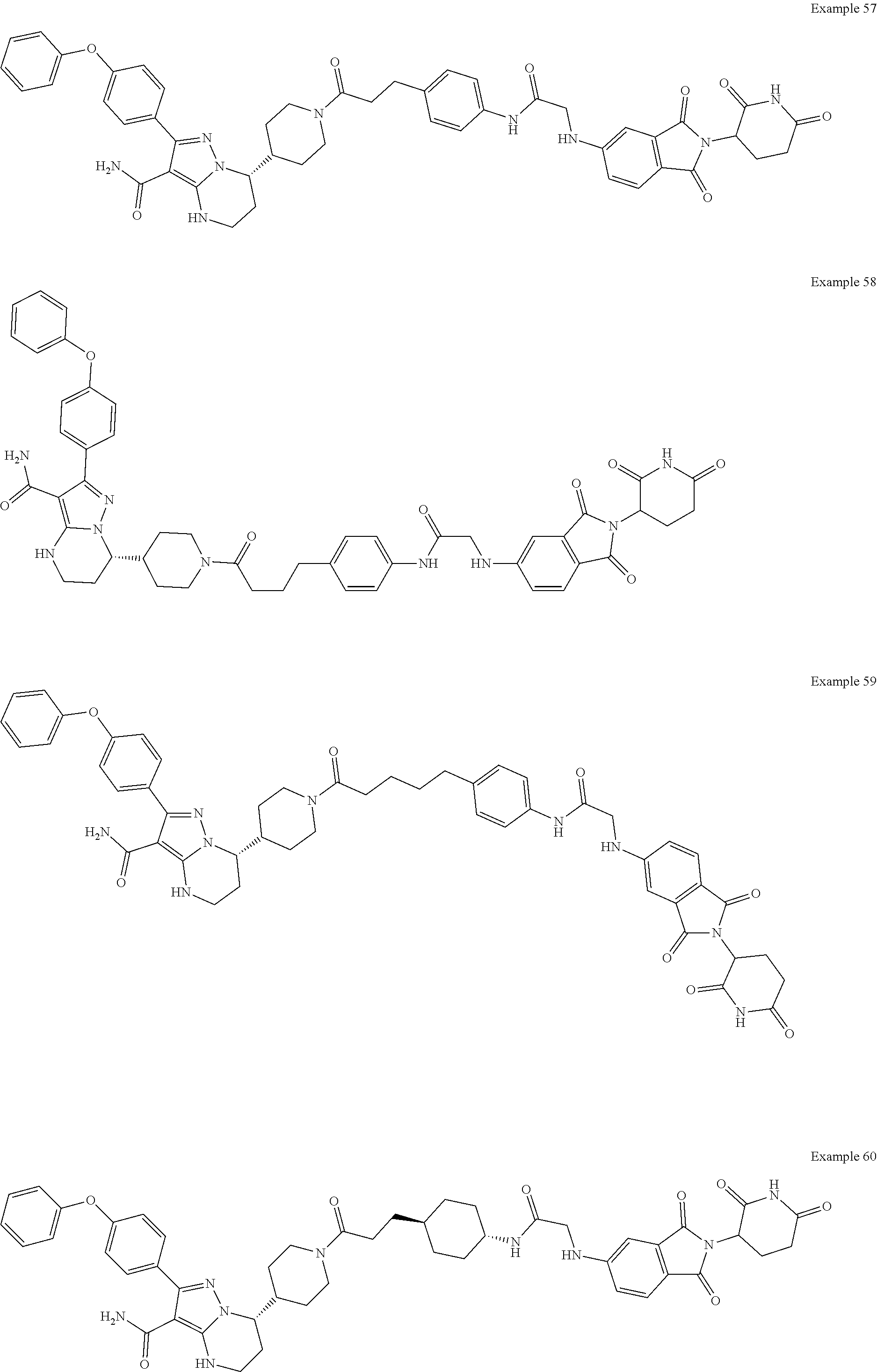 [SMILES: NC(=O)c1c(-c2ccc(Oc3ccccc3)cc2)nn2c1NCC[C@H]2C1CCN(C(=O)CCc2ccc(NC(=O)CNc3ccc4c(c3)C(=O)N(C3CCC(=O)NC3=O)C4=O)cc2)CC1 ; NC(=O)c1c(-c2ccc(Oc3ccccc3)cc2)nn2c1NCC[C@H]2C1CCN(C(=O)CCCc2ccc(NC(=O)CNc3ccc4c(c3)C(=O)N(C3CCC(=O)NC3=O)C4=O)cc2)CC1 ; NC(=O)c1c(-c2ccc(Oc3ccccc3)cc2)nn2c1NCC[C@H]2C1CCN(C(=O)CCCCc2ccc(NC(=O)CNc3ccc4c(c3)C(=O)N(C3CCC(=O)NC3=O)C4=O)cc2)CC1 ; NC(=O)c1c(-c2ccc(Oc3ccccc3)cc2)nn2c1NCC[C@H]2C1CCN(C(=O)CC[C@H]2CC[C@H](NC(=O)CNc3ccc4c(c3)C(=O)N(C3CCC(=O)NC3=O)C4=O)CC2)CC1]

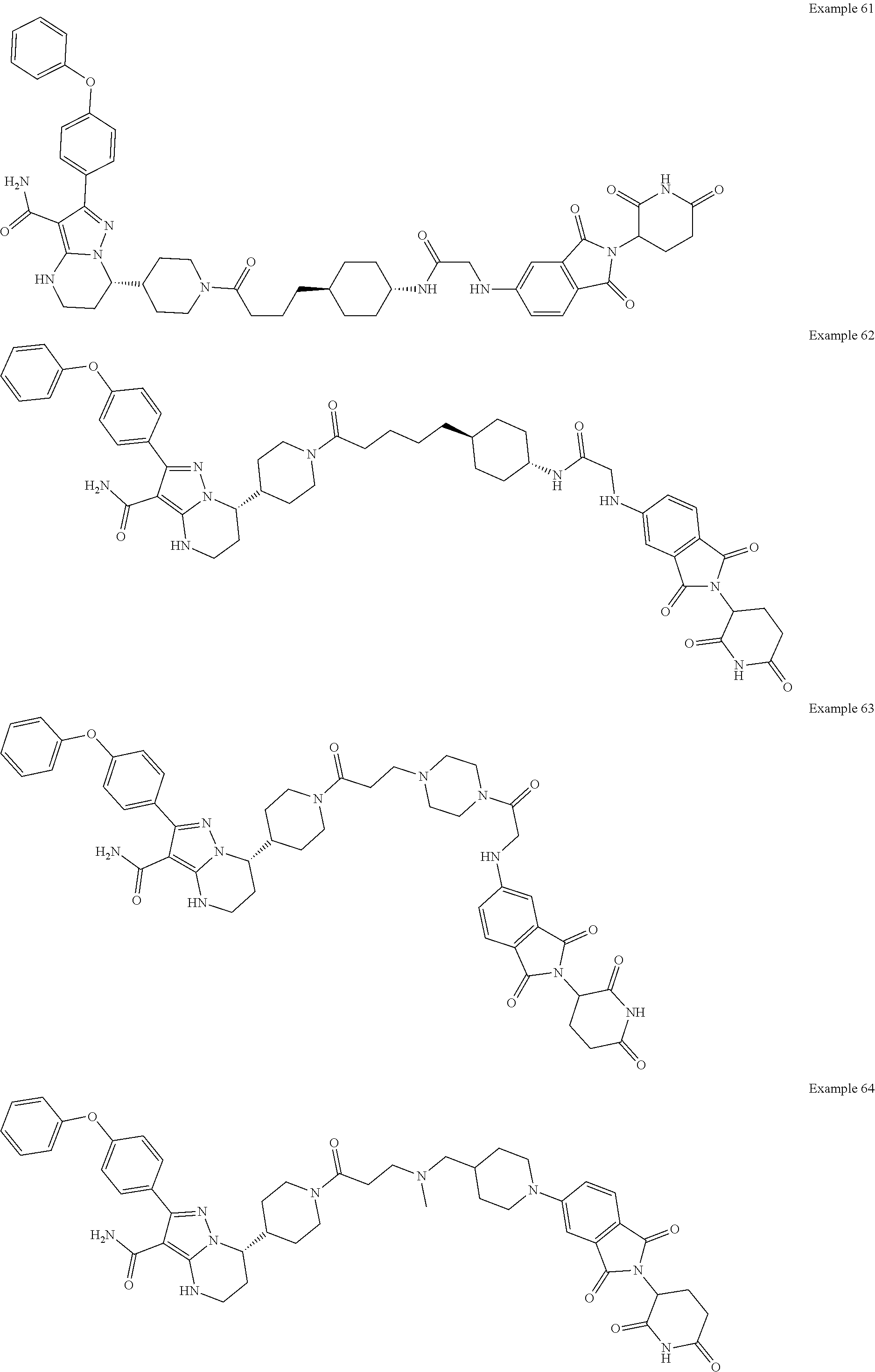

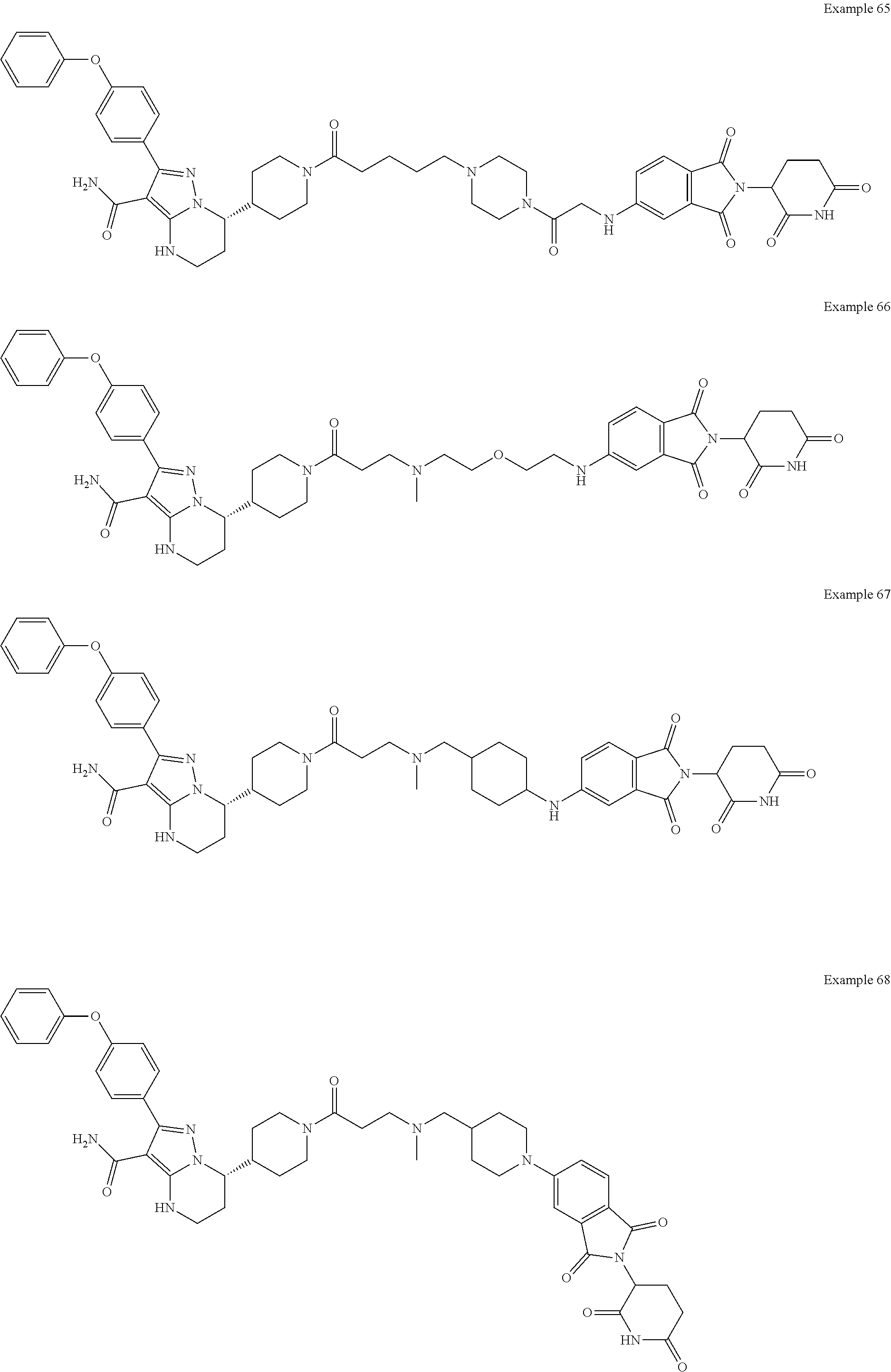 [SMILES: NC(=O)c1c(-c2ccc(Oc3ccccc3)cc2)nn2c1NCC[C@H]2C1CCN(C(=O)CCCCN2CCN(C(=O)CNc3ccc4c(c3)C(=O)N(C3CCC(=O)NC3=O)C4=O)CC2)CC1 ; CN(CCOCCNc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1 ; CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1 ; CN(CCC(=O)N1CCC([C@@H]2CCNc3c(C(N)=O)c(-c4ccc(Oc5ccccc5)cc4)nn32)CC1)CC1CCN(c2ccc3c(c2)C(=O)N(C2CCC(=O)NC2=O)C3=O)CC1]

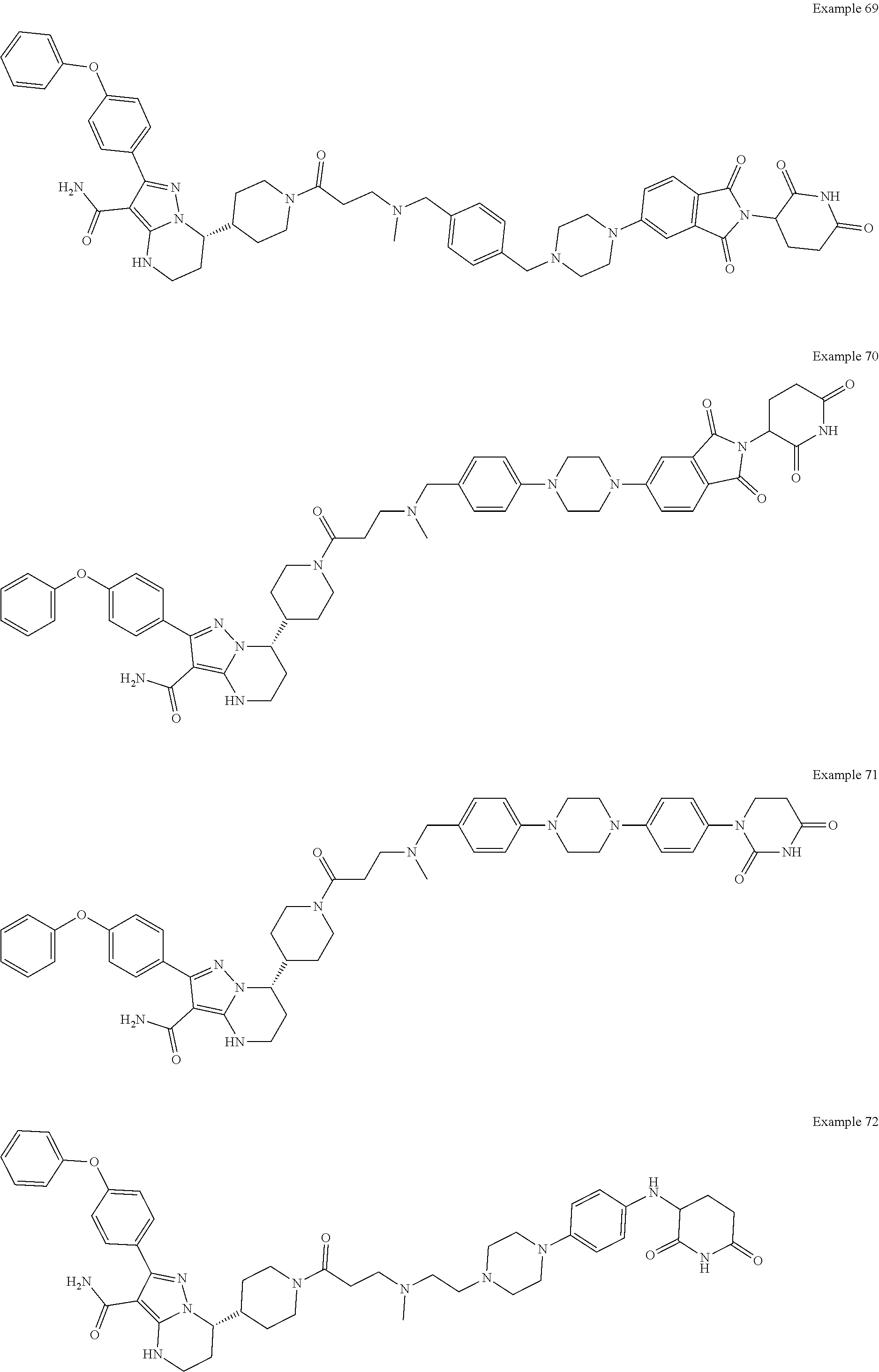 [SMILES: CN(CCC(=O)N1CCC([C@@H]2CCNc3c(C(N)=O)c(-c4ccc(Oc5ccccc5)cc4)nn32)CC1)Cc1ccc(CN2CCN(c3ccc4c(c3)C(=O)N(C3CCC(=O)NC3=O)C4=O)CC2)cc1 ; CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)N(C3CCC(=O)NC3=O)C4=O)CC2)cc1 ; CN(CCC(=O)N1CCC([C@@H]2CCNc3c(C(N)=O)c(-c4ccc(Oc5ccccc5)cc4)nn32)CC1)Cc1ccc(N2CCN(c3ccc(N4CCC(=O)NC4=O)cc3)CC2)cc1 ; CN(CCC(=O)N1CCC([C@@H]2CCNc3c(C(N)=O)c(-c4ccc(Oc5ccccc5)cc4)nn32)CC1)CCN1CCN(c2ccc(NC3CCC(=O)NC3=O)cc2)CC1]

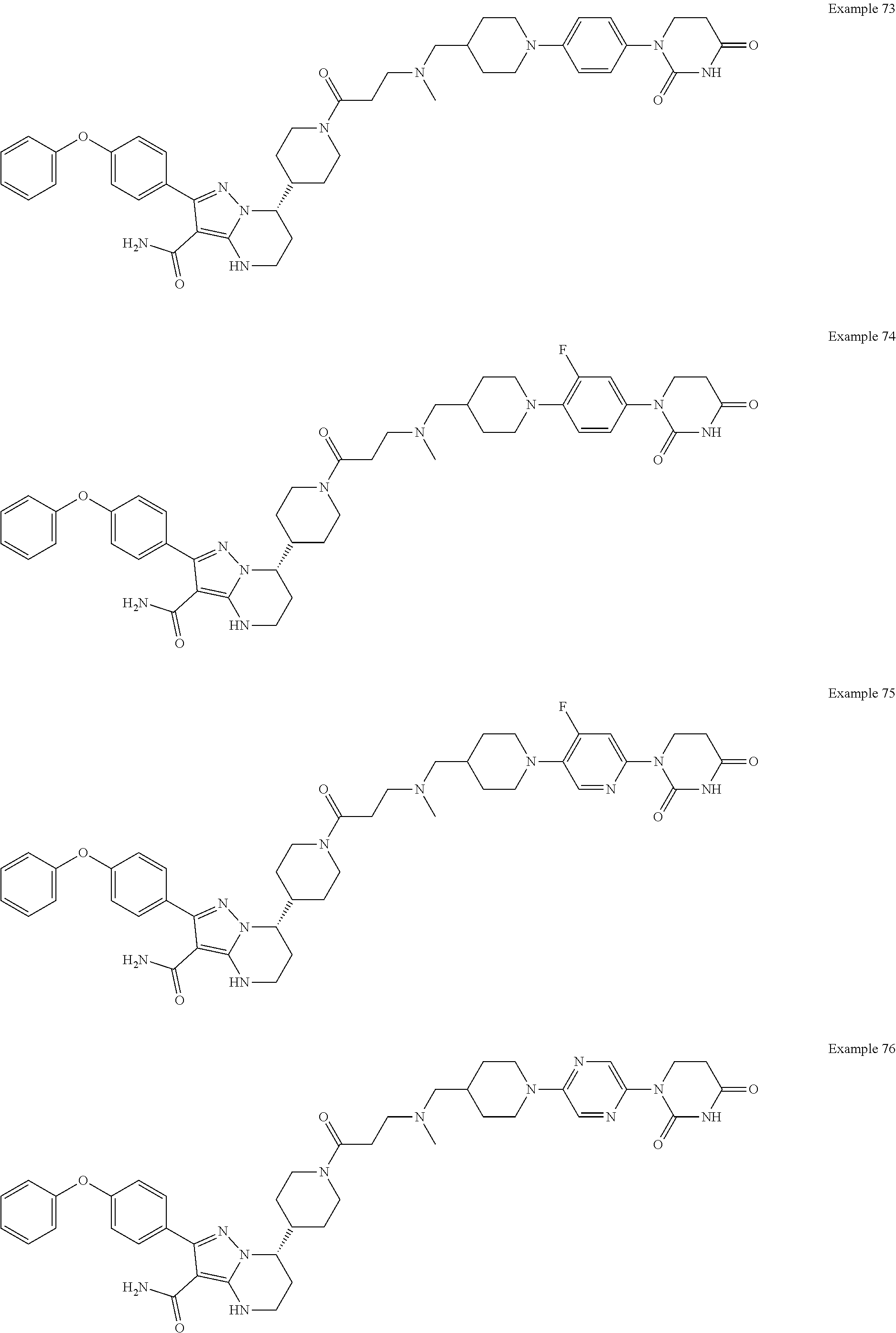 [SMILES: CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1 ; CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2F)CC1 ; CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cc2F)CC1 ; CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cn2)CC1]

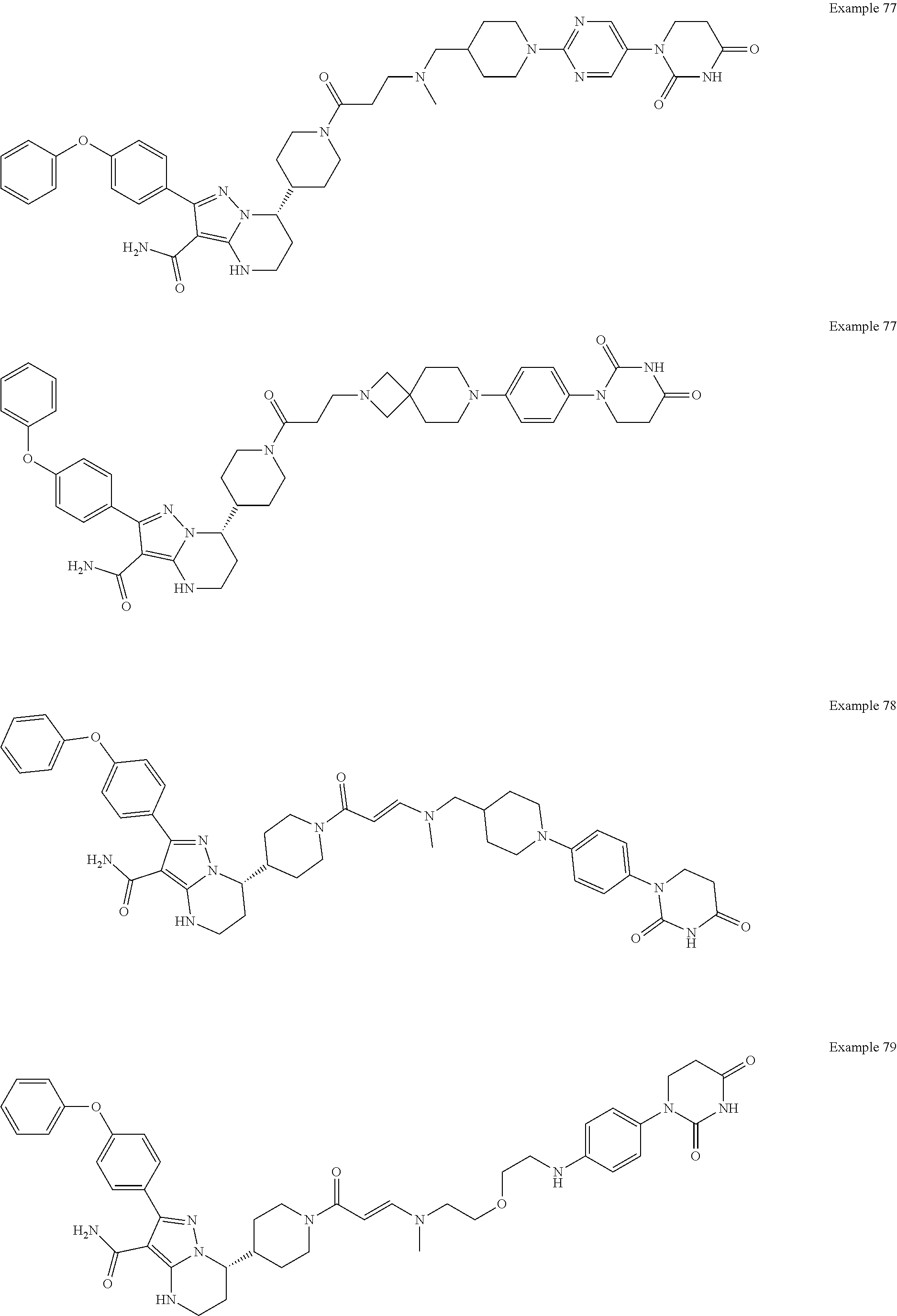 [SMILES: CN(CCC(=O)N1CCC([C@@H]2CCNc3c(C(N)=O)c(-c4ccc(Oc5ccccc5)cc4)nn32)CC1)CC1CCN(c2ncc(N3CCC(=O)NC3=O)cn2)CC1 ; NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1 ; CN(/C=C/C(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1 ; CN(/C=C/C(=O)N1CCC([C@@H]2CCNc3c(C(N)=O)c(-c4ccc(Oc5ccccc5)cc4)nn32)CC1)CCOCCNc1ccc(N2CCC(=O)NC2=O)cc1]

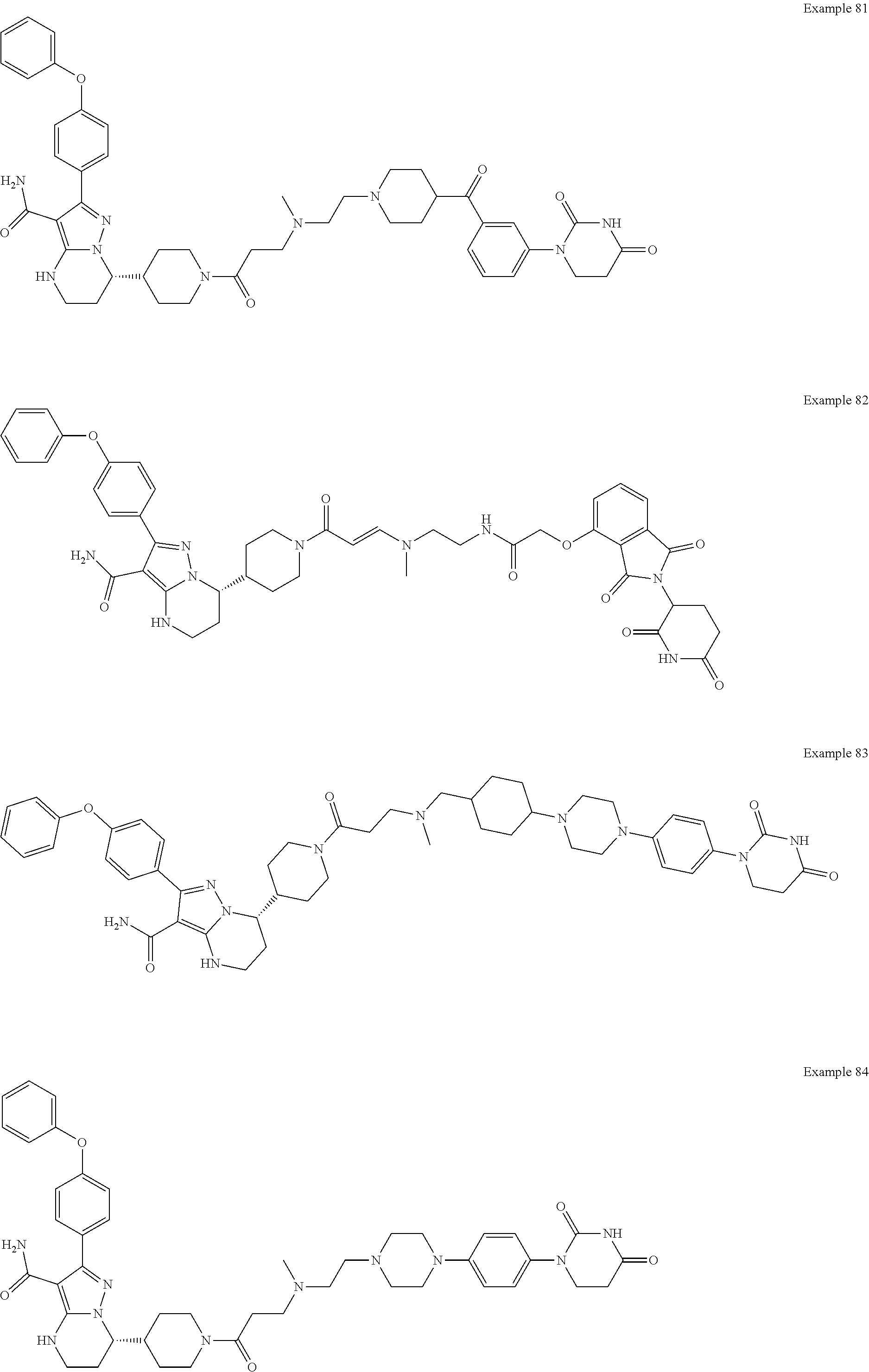 [SMILES: CN(CCC(=O)N1CCC([C@@H]2CCNc3c(C(N)=O)c(-c4ccc(Oc5ccccc5)cc4)nn32)CC1)CCN1CCC(C(=O)c2cccc(N3CCC(=O)NC3=O)c2)CC1 ; CN(/C=C/C(=O)N1CCC([C@@H]2CCNc3c(C(N)=O)c(-c4ccc(Oc5ccccc5)cc4)nn32)CC1)CCNC(=O)COc1cccc2c1C(=O)N(C1CCC(=O)NC1=O)C2=O ; CN(CCC(=O)N1CCC([C@@H]2CCNc3c(C(N)=O)c(-c4ccc(Oc5ccccc5)cc4)nn32)CC1)CC1CCC(N2CCN(c3ccc(N4CCC(=O)NC4=O)cc3)CC2)CC1 ; CN(CCC(=O)N1CCC([C@@H]2CCNc3c(C(N)=O)c(-c4ccc(Oc5ccccc5)cc4)nn32)CC1)CCN1CCN(c2ccc(N3CCC(=O)NC3=O)cc2)CC1]

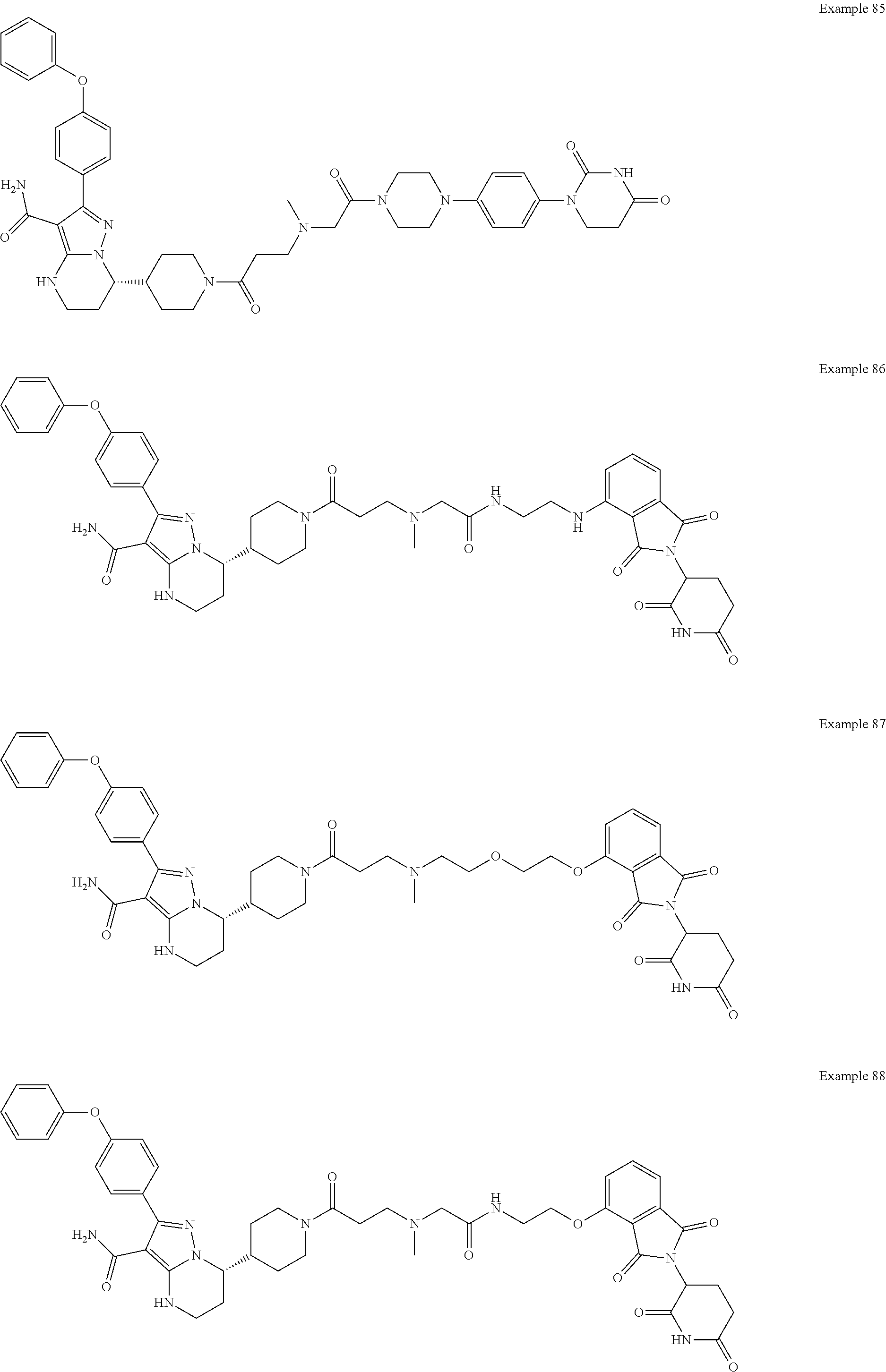 [SMILES: CN(CCC(=O)N1CCC([C@@H]2CCNc3c(C(N)=O)c(-c4ccc(Oc5ccccc5)cc4)nn32)CC1)CC(=O)N1CCN(c2ccc(N3CCC(=O)NC3=O)cc2)CC1 ; CN(CCC(=O)N1CCC([C@@H]2CCNc3c(C(N)=O)c(-c4ccc(Oc5ccccc5)cc4)nn32)CC1)CC(=O)NCCNc1cccc2c1C(=O)N(C1CCC(=O)NC1=O)C2=O ; CN(CCOCCOc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1 ; CN(CCC(=O)N1CCC([C@@H]2CCNc3c(C(N)=O)c(-c4ccc(Oc5ccccc5)cc4)nn32)CC1)CC(=O)NCCOc1cccc2c1C(=O)N(C1CCC(=O)NC1=O)C2=O]

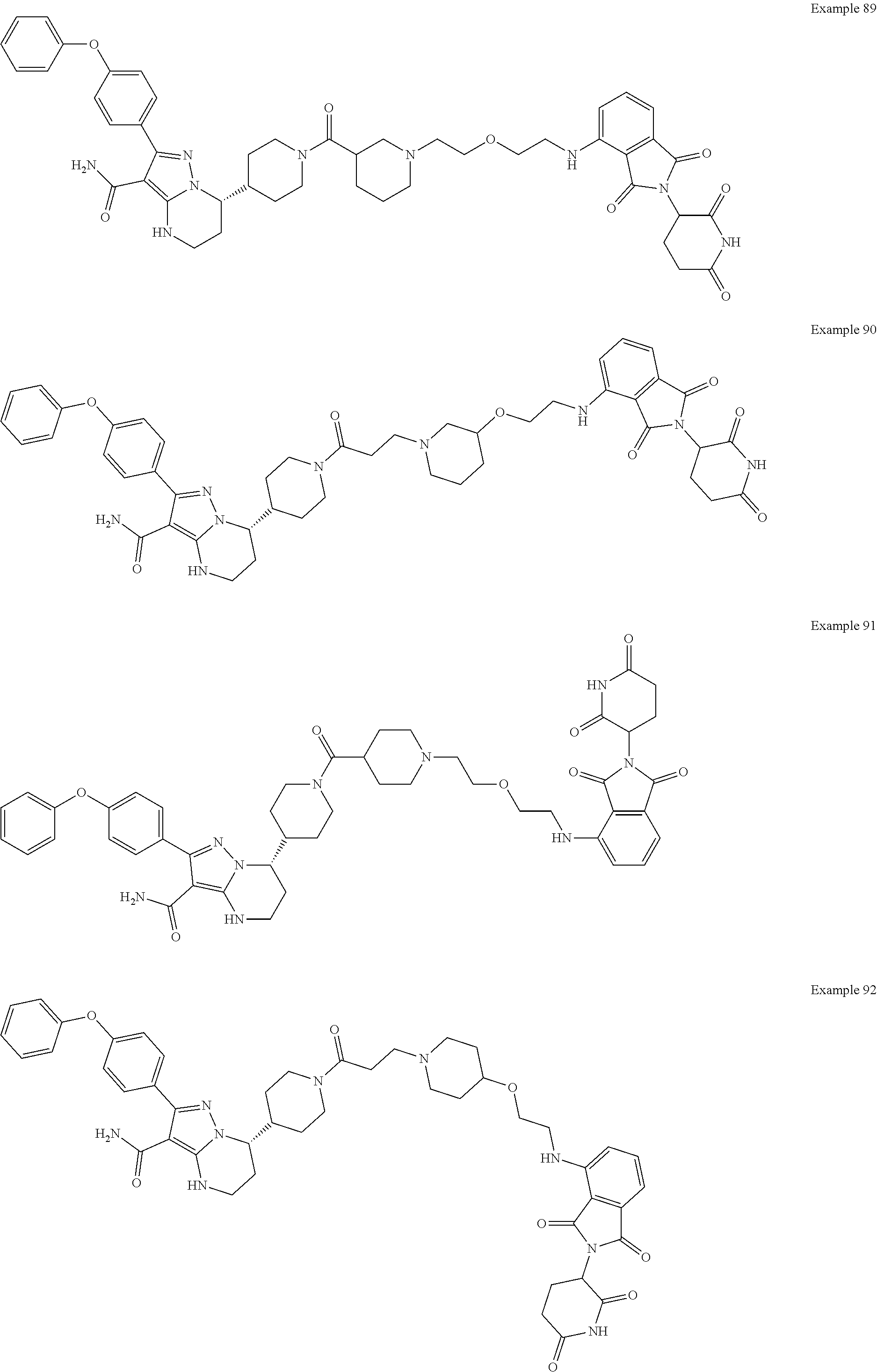 [SMILES: NC(=O)c1c(-c2ccc(Oc3ccccc3)cc2)nn2c1NCC[C@H]2C1CCN(C(=O)C2CCCN(CCOCCNc3cccc4c3C(=O)N(C3CCC(=O)NC3=O)C4=O)C2)CC1 ; NC(=O)c1c(-c2ccc(Oc3ccccc3)cc2)nn2c1NCC[C@H]2C1CCN(C(=O)CCN2CCCC(OCCNc3cccc4c3C(=O)N(C3CCC(=O)NC3=O)C4=O)C2)CC1 ; NC(=O)c1c(-c2ccc(Oc3ccccc3)cc2)nn2c1NCC[C@H]2C1CCN(C(=O)C2CCN(CCOCCNc3cccc4c3C(=O)N(C3CCC(=O)NC3=O)C4=O)CC2)CC1 ; NC(=O)c1c(-c2ccc(Oc3ccccc3)cc2)nn2c1NCC[C@H]2C1CCN(C(=O)CCN2CCC(OCCNc3cccc4c3C(=O)N(C3CCC(=O)NC3=O)C4=O)CC2)CC1]

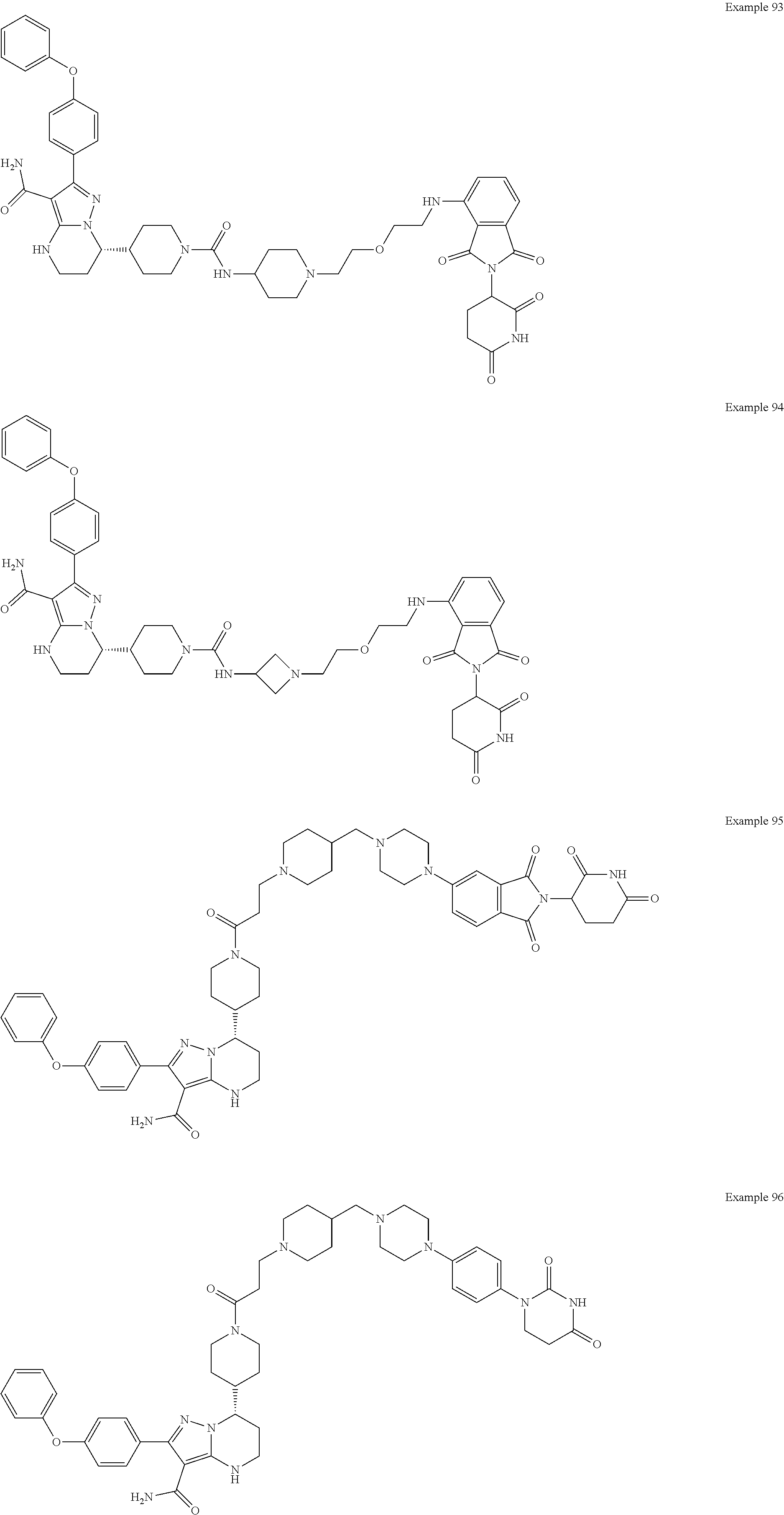 [SMILES: NC(=O)c1c(-c2ccc(Oc3ccccc3)cc2)nn2c1NCC[C@H]2C1CCN(C(=O)NC2CCN(CCOCCNc3cccc4c3C(=O)N(C3CCC(=O)NC3=O)C4=O)CC2)CC1 ; NC(=O)c1c(-c2ccc(Oc3ccccc3)cc2)nn2c1NCC[C@H]2C1CCN(C(=O)NC2CN(CCOCCNc3cccc4c3C(=O)N(C3CCC(=O)NC3=O)C4=O)C2)CC1 ; NC(=O)c1c(-c2ccc(Oc3ccccc3)cc2)nn2c1NCC[C@H]2C1CCN(C(=O)CCN2CCC(CN3CCN(c4ccc5c(c4)C(=O)N(C4CCC(=O)NC4=O)C5=O)CC3)CC2)CC1 ; NC(=O)c1c(-c2ccc(Oc3ccccc3)cc2)nn2c1NCC[C@H]2C1CCN(C(=O)CCN2CCC(CN3CCN(c4ccc(N5CCC(=O)NC5=O)cc4)CC3)CC2)CC1]

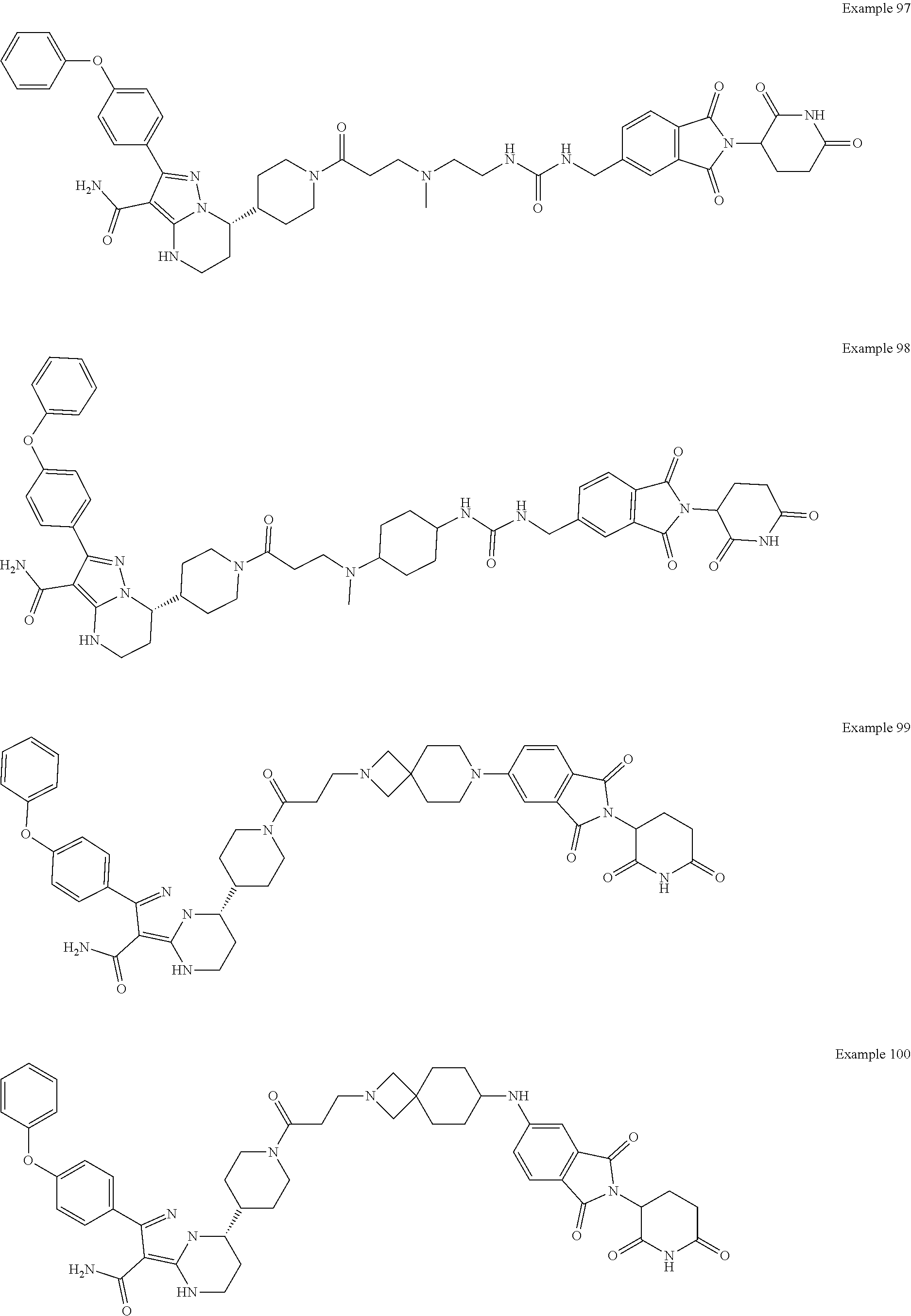 [SMILES: CN(CCNC(=O)NCc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1 ; CN(CCC(=O)N1CCC([C@@H]2CCNc3c(C(N)=O)c(-c4ccc(Oc5ccccc5)cc4)nn32)CC1)C1CCC(NC(=O)NCc2ccc3c(c2)C(=O)N(C2CCC(=O)NC2=O)C3=O)CC1 ; N=C(/C(C(N)=O)=C1/NCC[C@@H](C2CCN(C(=O)CCN3CC4(CCN(c5ccc6c(c5)C(=O)N(C5CCC(=O)NC5=O)C6=O)CC4)C3)CC2)N1)c1ccc(Oc2ccccc2)cc1 ; N=C(/C(C(N)=O)=C1/NCC[C@@H](C2CCN(C(=O)CCN3CC4(CCC(Nc5ccc6c(c5)C(=O)N(C5CCC(=O)NC5=O)C6=O)CC4)C3)CC2)N1)c1ccc(Oc2ccccc2)cc1]

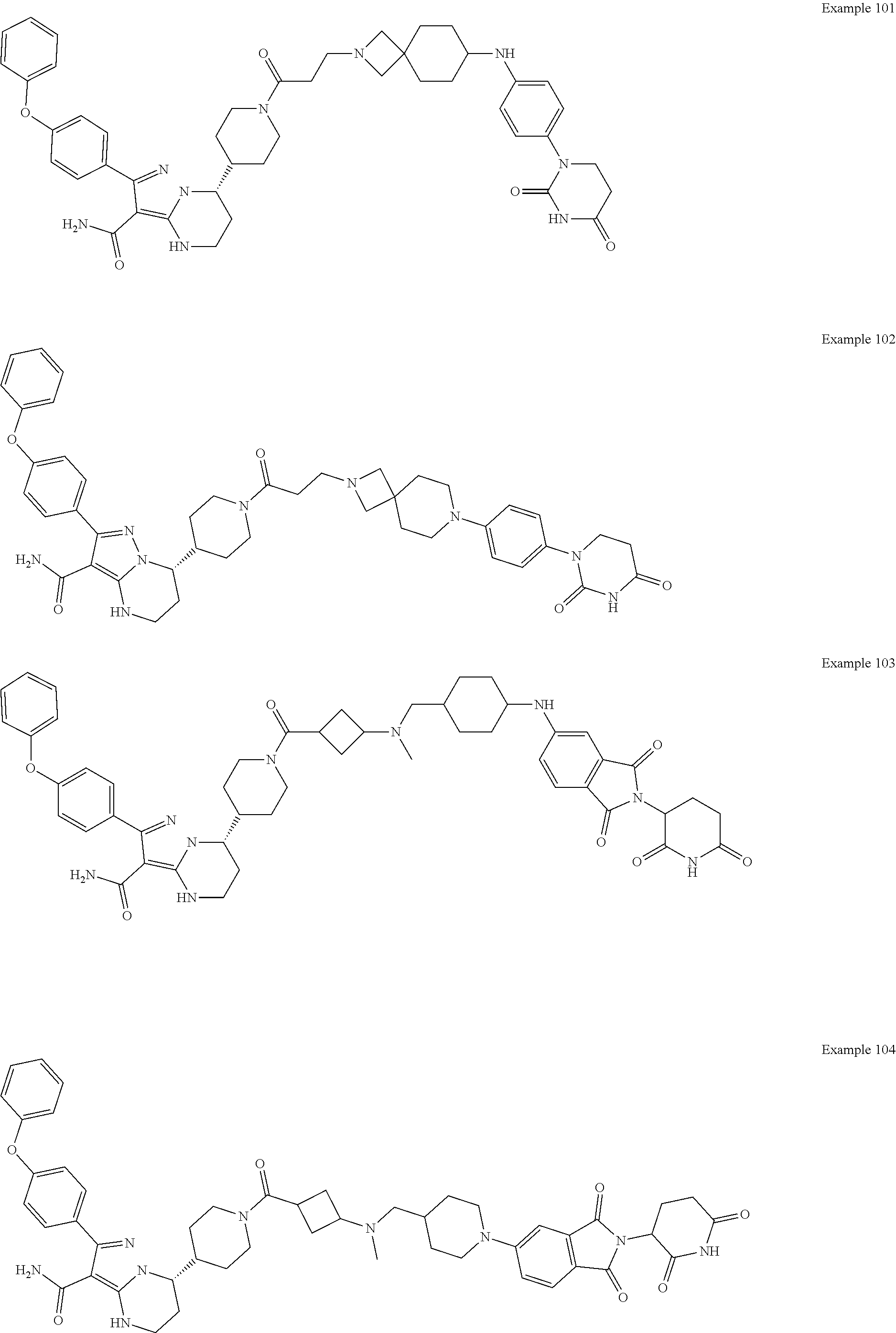 [SMILES: N=C(/C(C(N)=O)=C1/NCC[C@@H](C2CCN(C(=O)CCN3CC4(CCC(Nc5ccc(N6CCC(=O)NC6=O)cc5)CC4)C3)CC2)N1)c1ccc(Oc2ccccc2)cc1 ; NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1 ; CN(CC1CCC(Nc2ccc3c(c2)C(=O)N(C2CCC(=O)NC2=O)C3=O)CC1)C1CC(C(=O)N2CCC([C@@H]3CCN/C(=C(/C(=N)c4ccc(Oc5ccccc5)cc4)C(N)=O)N3)CC2)C1 ; CN(CC1CCN(c2ccc3c(c2)C(=O)N(C2CCC(=O)NC2=O)C3=O)CC1)C1CC(C(=O)N2CCC([C@@H]3CCN/C(=C(/C(=N)c4ccc(Oc5ccccc5)cc4)C(N)=O)N3)CC2)C1]

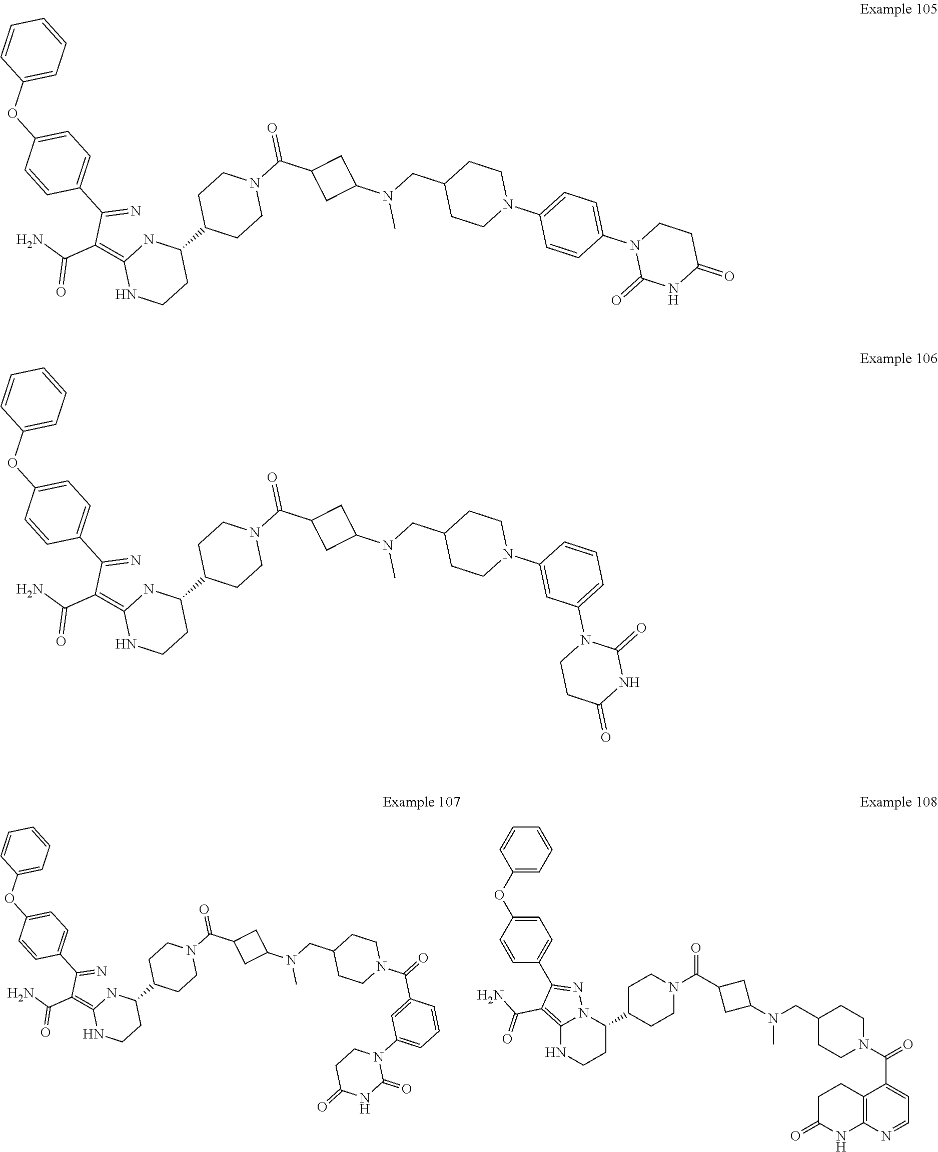 [SMILES: CN(CC1CCN(c2ccc(N3CCC(=O)NC3=O)cc2)CC1)C1CC(C(=O)N2CCC([C@@H]3CCN/C(=C(/C(=N)c4ccc(Oc5ccccc5)cc4)C(N)=O)N3)CC2)C1 ; CN(CC1CCN(c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1 ; CN(CC1CCN(C(=O)c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1 ; CN(CC1CCN(C(=O)c2ccnc3c2CCC(=O)N3)CC1)C1CC(C(=O)N2CCC([C@@H]3CCNc4c(C(N)=O)c(-c5ccc(Oc6ccccc6)cc5)nn43)CC2)C1]

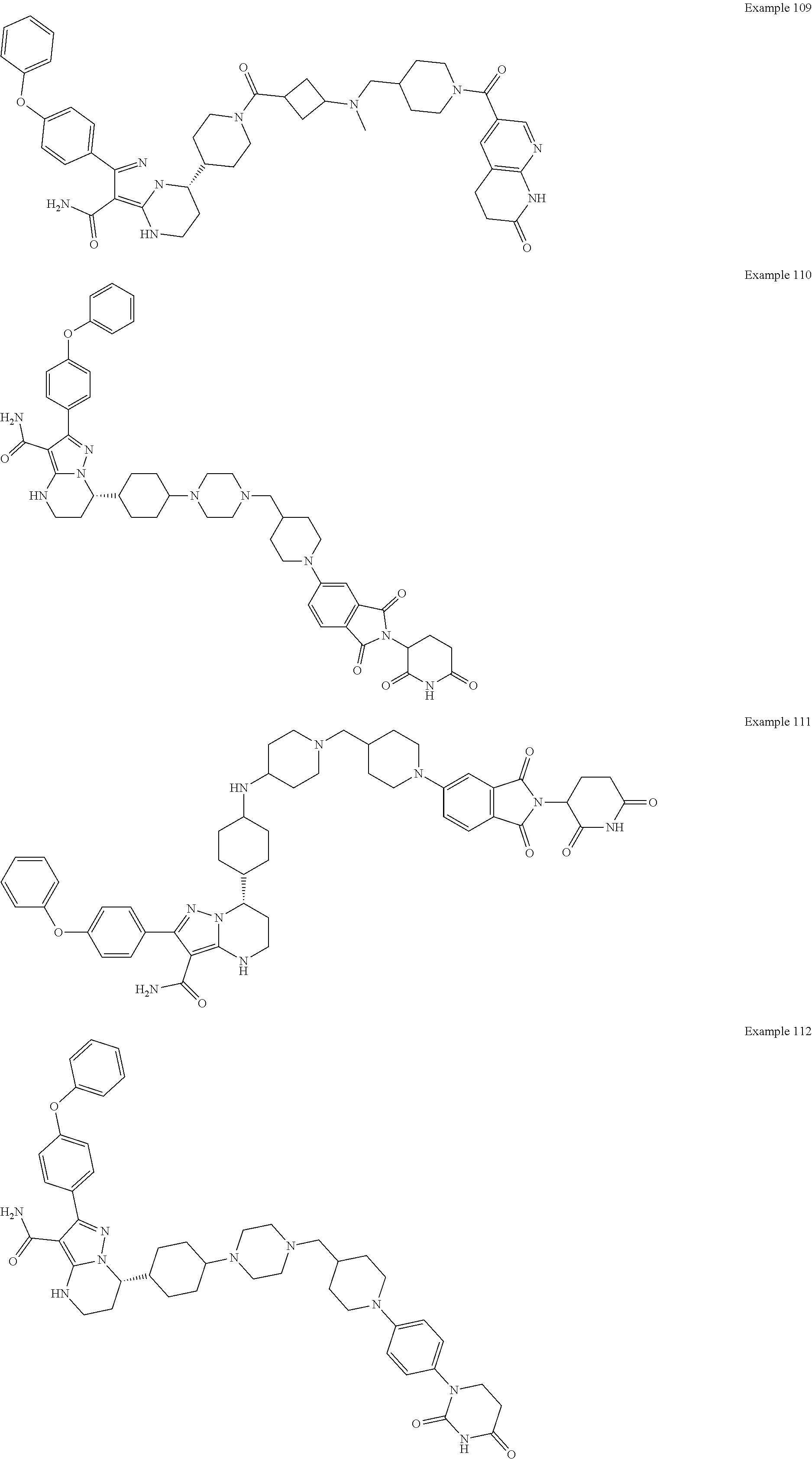 [SMILES: CN(CC1CCN(C(=O)c2cnc3c(c2)CCC(=O)N3)CC1)C1CC(C(=O)N2CCC([C@@H]3CCN/C(=C(/C(=N)c4ccc(Oc5ccccc5)cc4)C(N)=O)N3)CC2)C1 ; NC(=O)c1c(-c2ccc(Oc3ccccc3)cc2)nn2c1NCC[C@H]2C1CCC(N2CCN(CC3CCN(c4ccc5c(c4)C(=O)N(C4CCC(=O)NC4=O)C5=O)CC3)CC2)CC1 ; NC(=O)c1c(-c2ccc(Oc3ccccc3)cc2)nn2c1NCC[C@H]2C1CCC(NC2CCN(CC3CCN(c4ccc5c(c4)C(=O)N(C4CCC(=O)NC4=O)C5=O)CC3)CC2)CC1 ; NC(=O)c1c(-c2ccc(Oc3ccccc3)cc2)nn2c1NCC[C@H]2C1CCC(N2CCN(CC3CCN(c4ccc(N5CCC(=O)NC5=O)cc4)CC3)CC2)CC1]

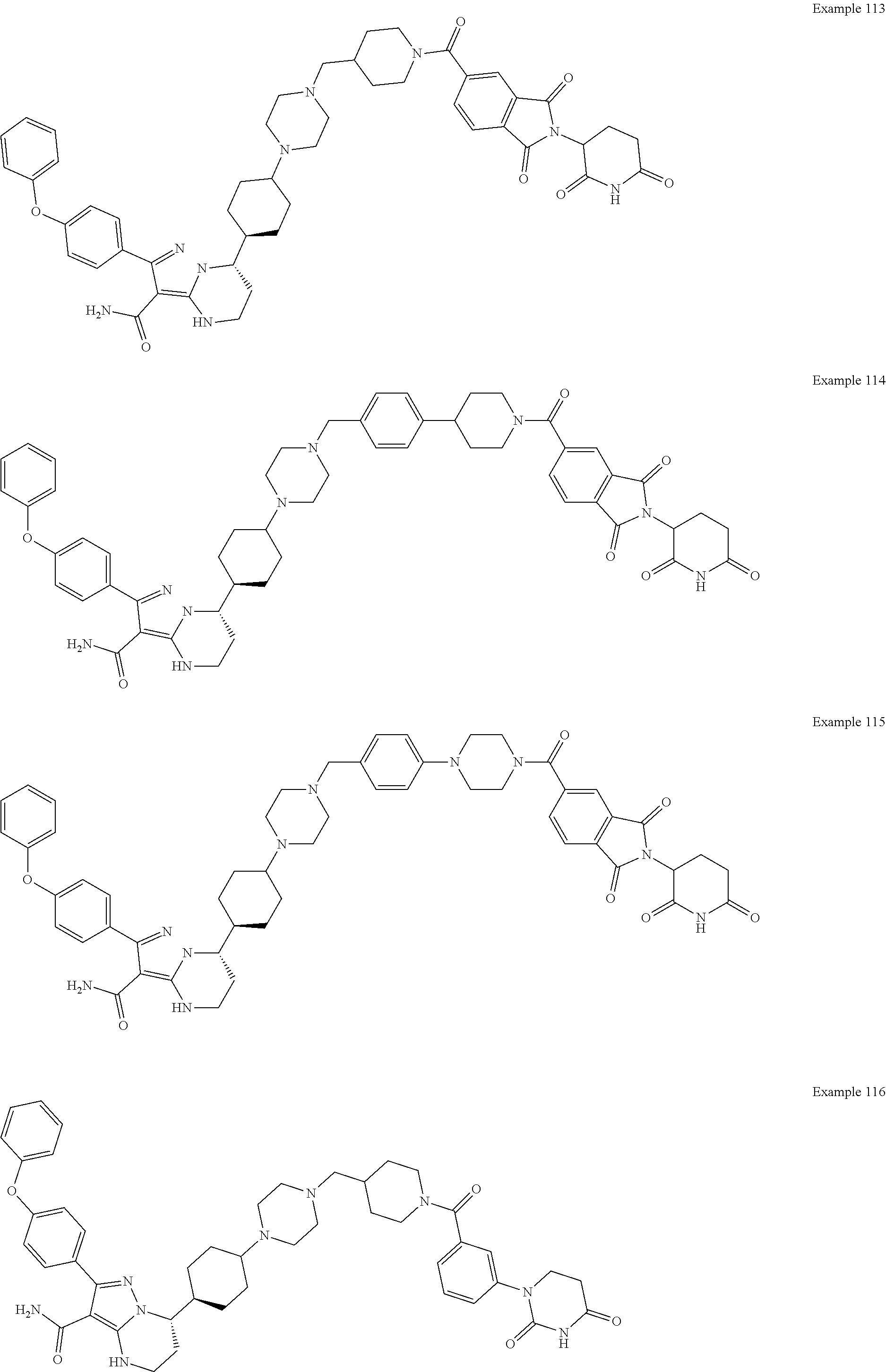

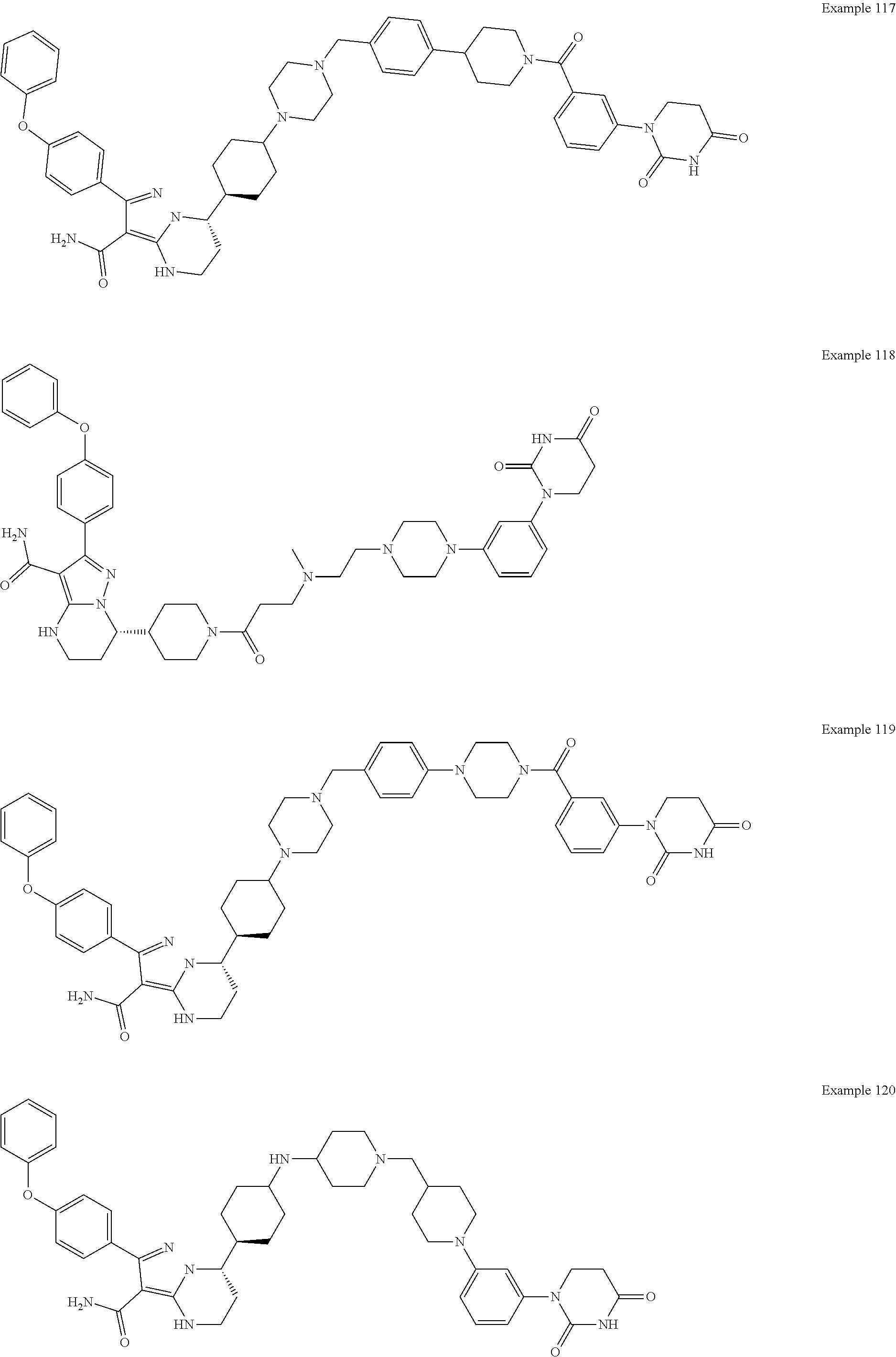

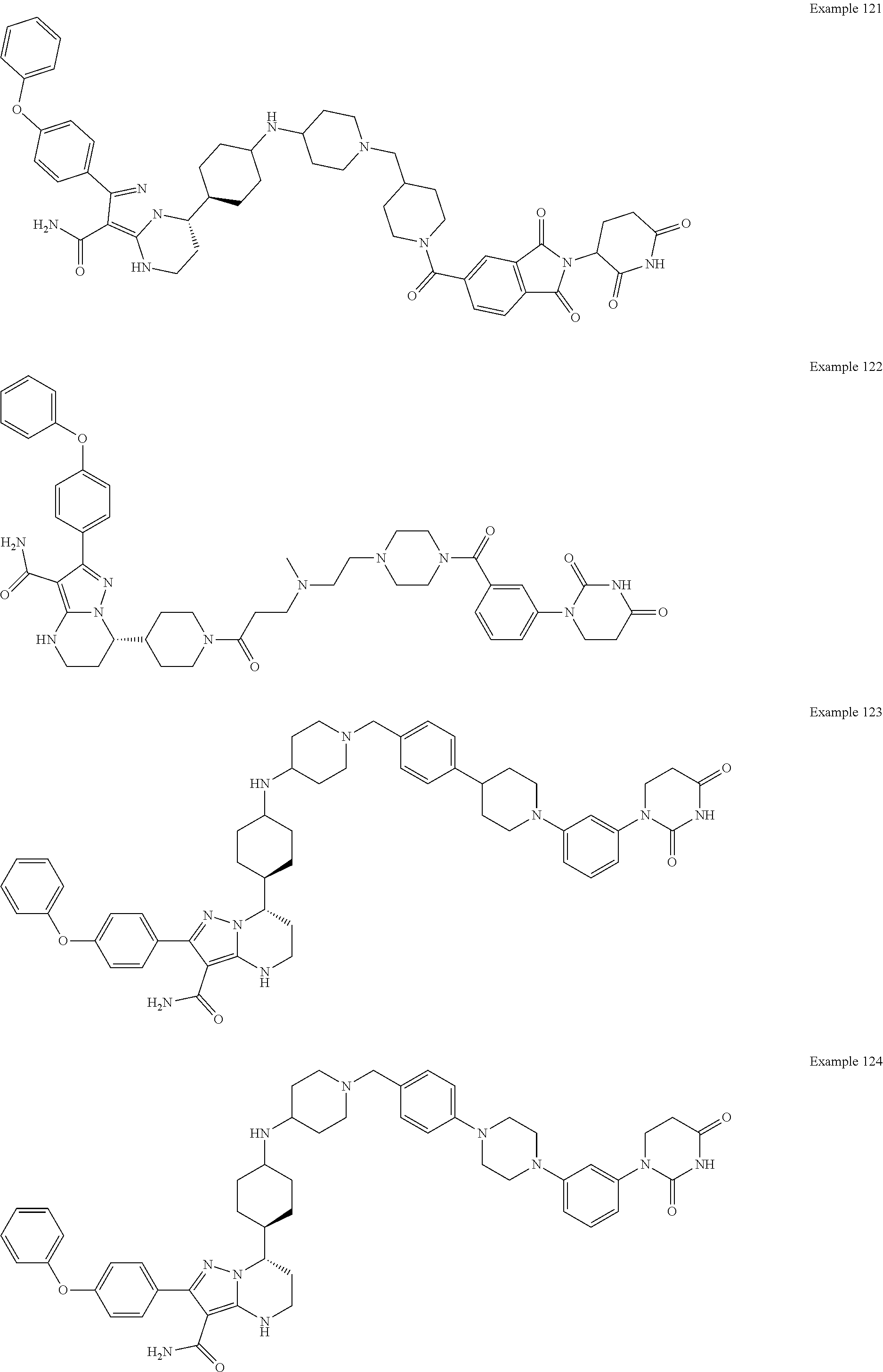

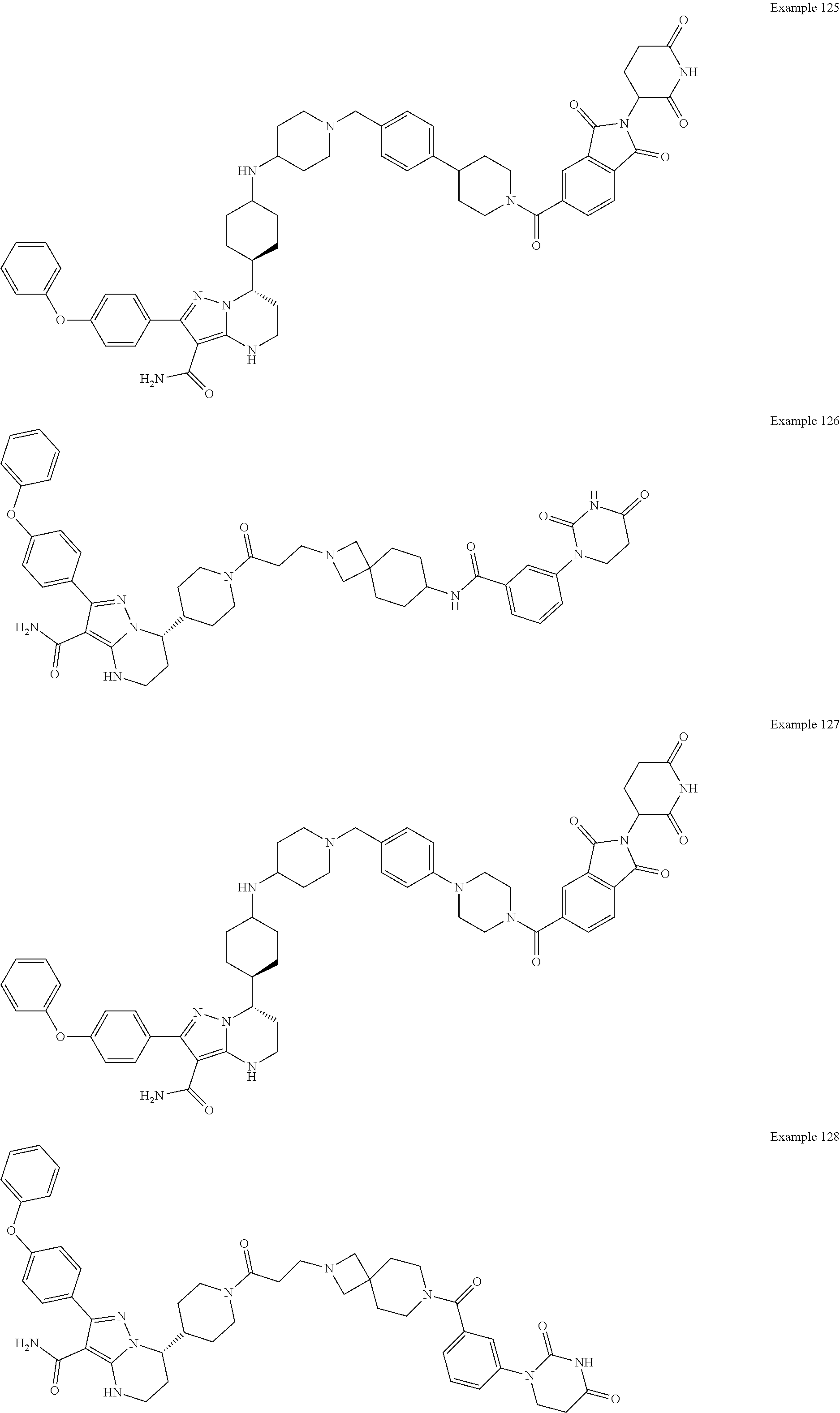

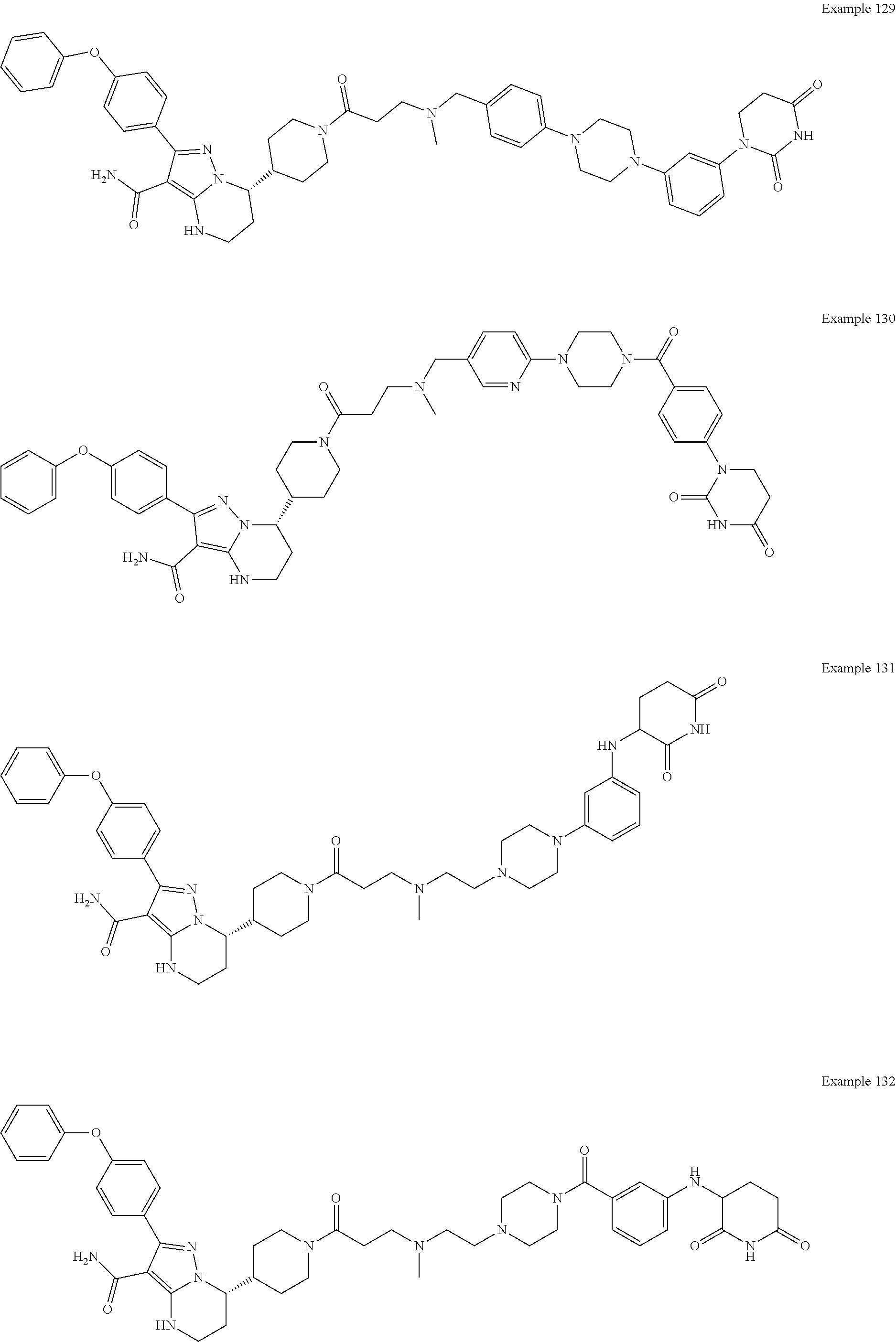 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3cccc(N4CCC(=O)NC4=O)c3)CC2)cc1 ; CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)nc1 ; CN(CCC(=O)N1CCC([C@@H]2CCNc3c(C(N)=O)c(-c4ccc(Oc5ccccc5)cc4)nn32)CC1)CCN1CCN(c2cccc(NC3CCC(=O)NC3=O)c2)CC1 ; CN(CCC(=O)N1CCC([C@@H]2CCNc3c(C(N)=O)c(-c4ccc(Oc5ccccc5)cc4)nn32)CC1)CCN1CCN(C(=O)c2cccc(NC3CCC(=O)NC3=O)c2)CC1]

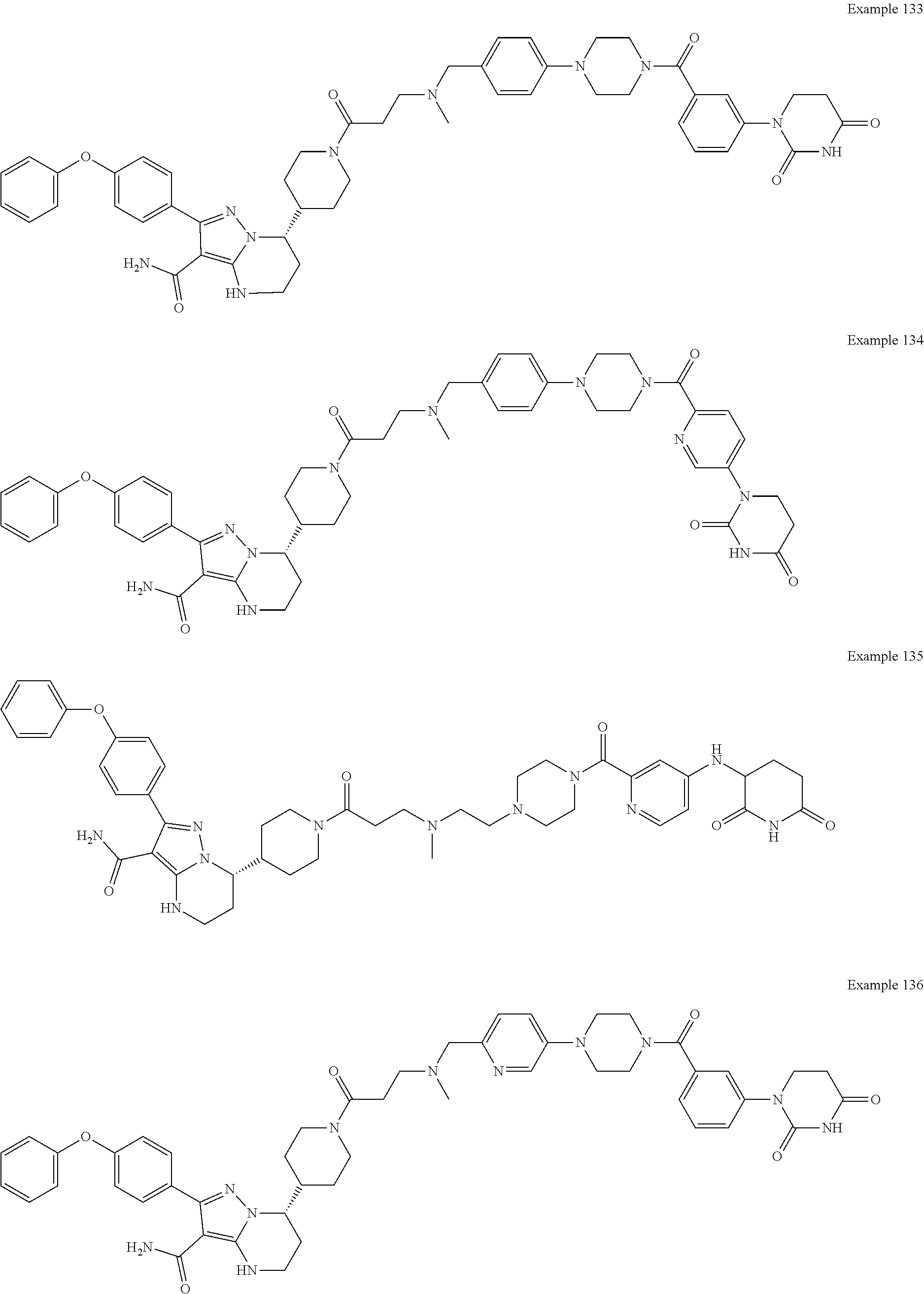 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cc1 ; CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cn3)CC2)cc1 ; CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccn2)CC1 ; CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cn1]

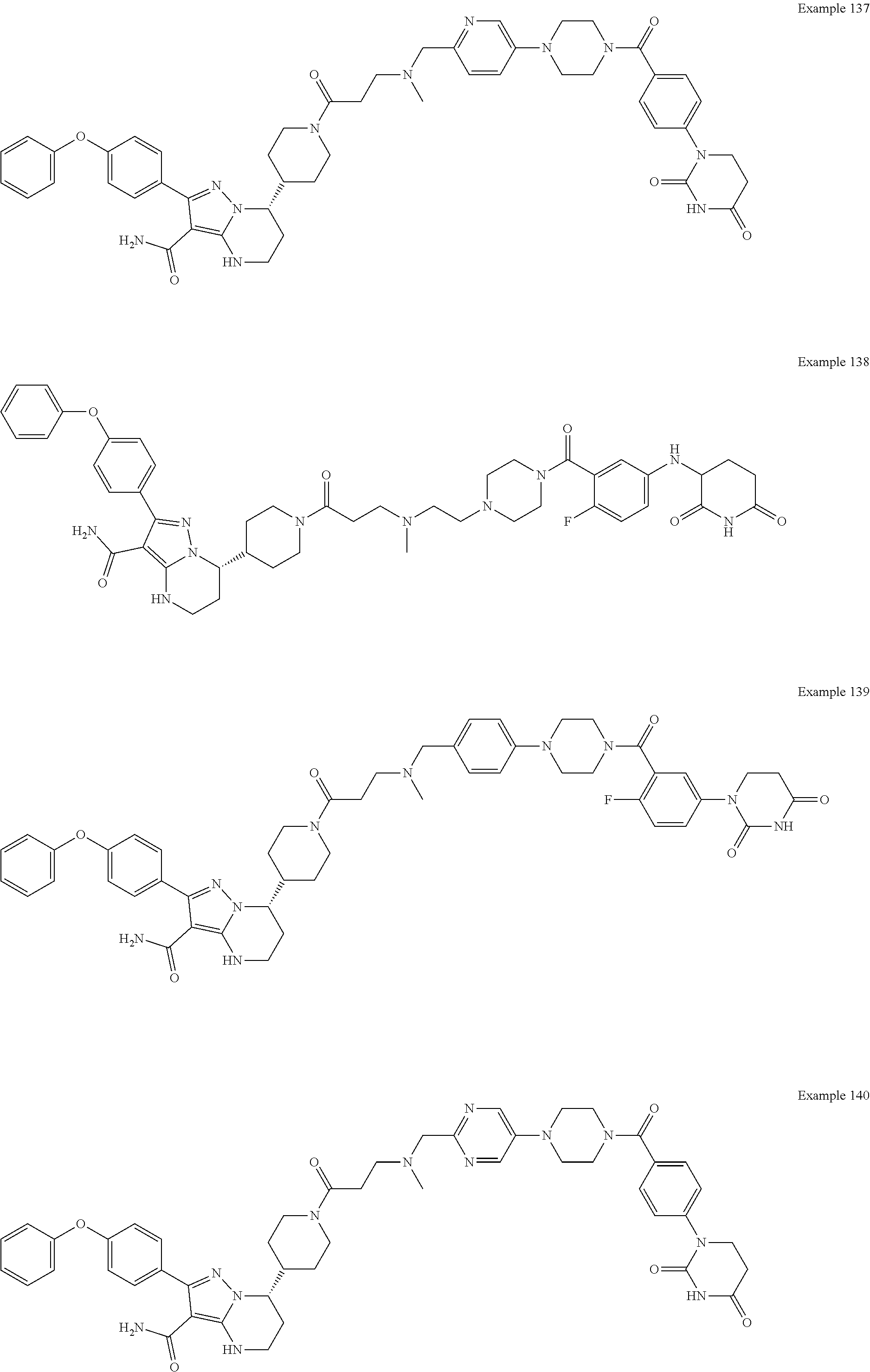 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)cn1 ; CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccc2F)CC1 ; CN(CCC(=O)N1CCC([C@@H]2CCNc3c(C(N)=O)c(-c4ccc(Oc5ccccc5)cc4)nn32)CC1)Cc1ccc(N2CCN(C(=O)c3cc(N4CCC(=O)NC4=O)ccc3F)CC2)cc1 ; CN(CCC(=O)N1CCC([C@@H]2CCNc3c(C(N)=O)c(-c4ccc(Oc5ccccc5)cc4)nn32)CC1)Cc1ncc(N2CCN(C(=O)c3ccc(N4CCC(=O)NC4=O)cc3)CC2)cn1]

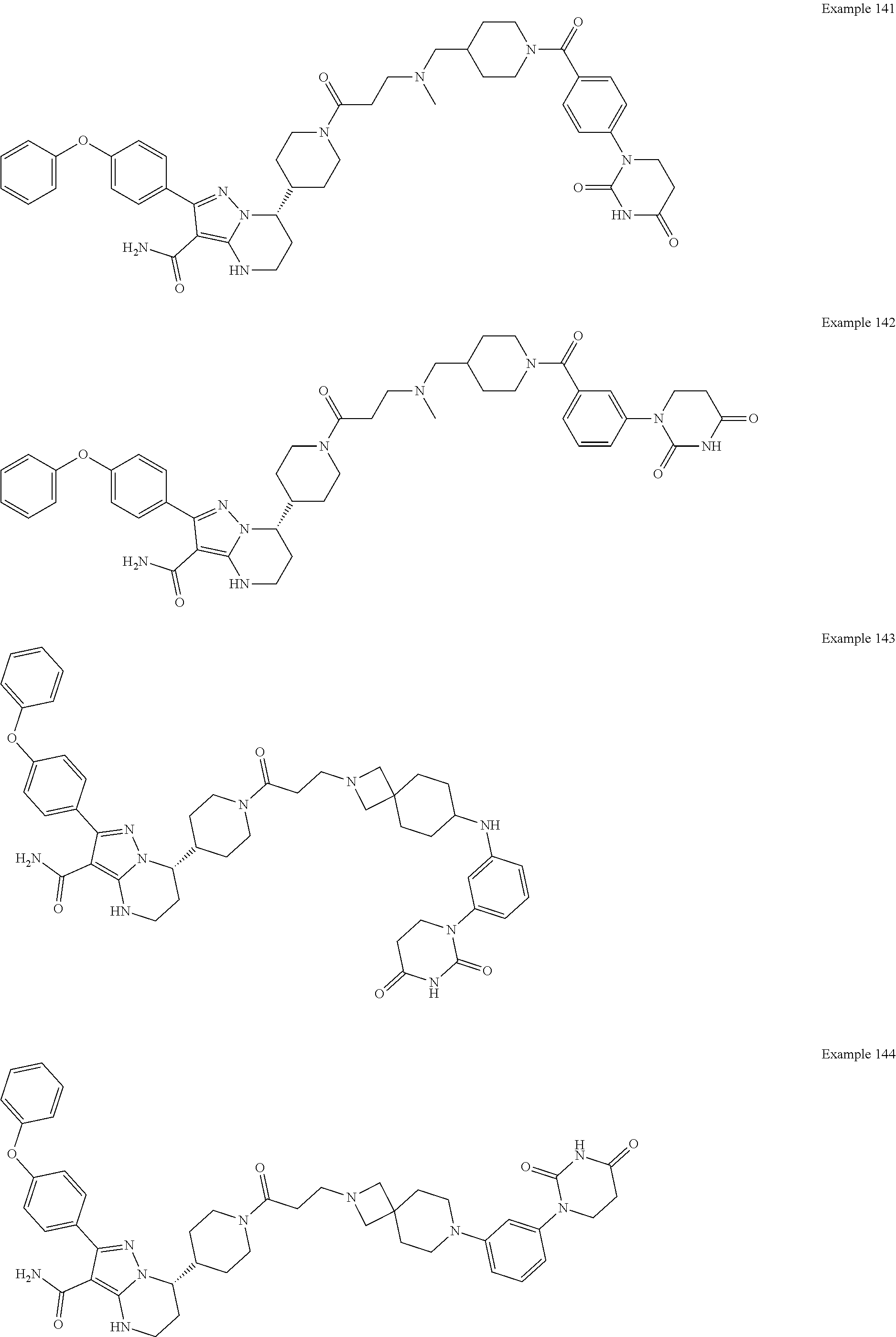 [SMILES: CN(CCC(=O)N1CCC([C@@H]2CCNc3c(C(N)=O)c(-c4ccc(Oc5ccccc5)cc4)nn32)CC1)CC1CCN(C(=O)c2ccc(N3CCC(=O)NC3=O)cc2)CC1 ; CN(CCC(=O)N1CCC([C@@H]2CCNc3c(C(N)=O)c(-c4ccc(Oc5ccccc5)cc4)nn32)CC1)CC1CCN(C(=O)c2cccc(N3CCC(=O)NC3=O)c2)CC1 ; NC(=O)c1c(-c2ccc(Oc3ccccc3)cc2)nn2c1NCC[C@H]2C1CCN(C(=O)CCN2CC3(CCC(Nc4cccc(N5CCC(=O)NC5=O)c4)CC3)C2)CC1 ; NC(=O)c1c(-c2ccc(Oc3ccccc3)cc2)nn2c1NCC[C@H]2C1CCN(C(=O)CCN2CC3(CCN(c4cccc(N5CCC(=O)NC5=O)c4)CC3)C2)CC1]

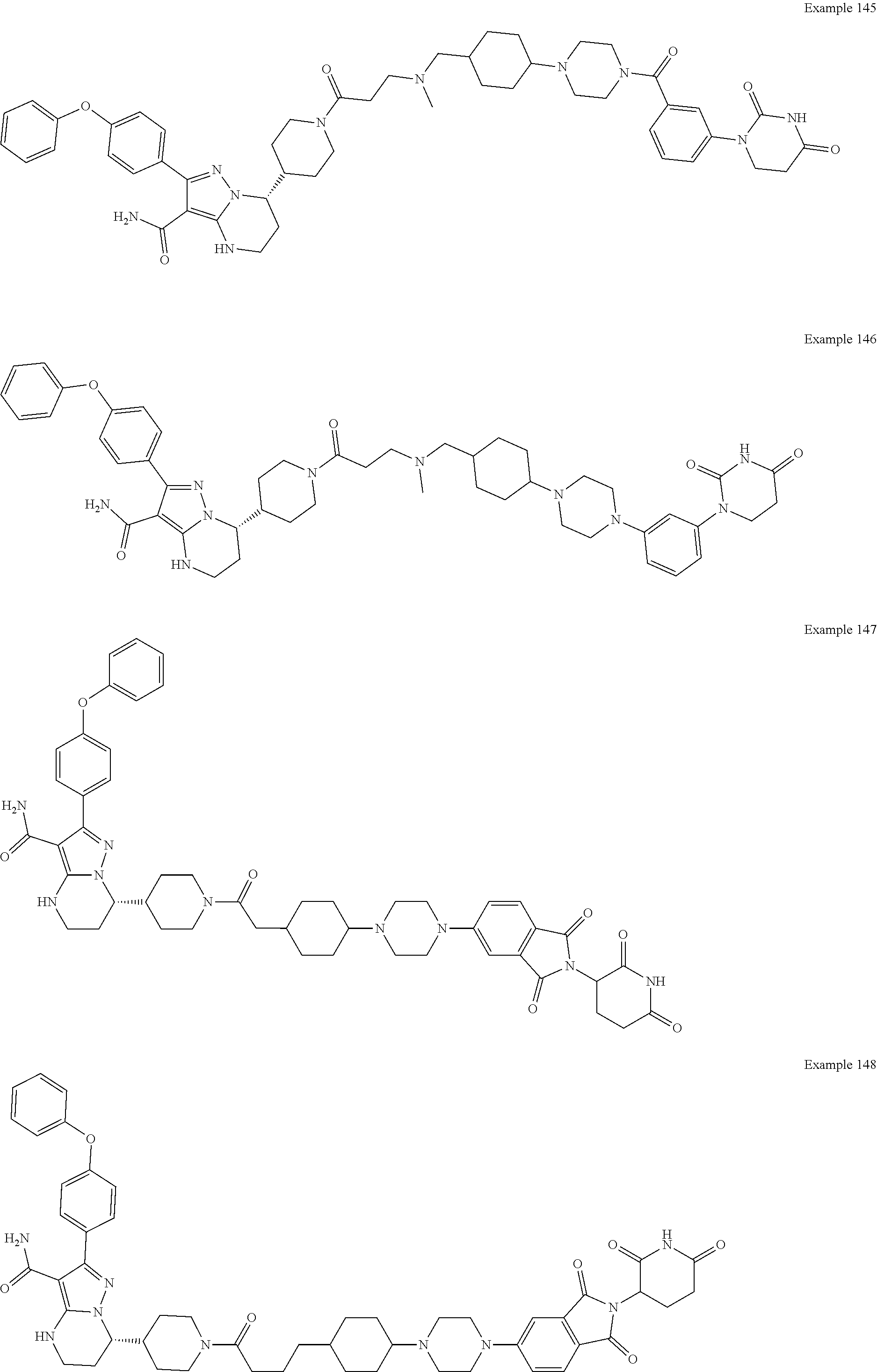 [SMILES: CN(CCC(=O)N1CCC([C@@H]2CCNc3c(C(N)=O)c(-c4ccc(Oc5ccccc5)cc4)nn32)CC1)CC1CCC(N2CCN(C(=O)c3cccc(N4CCC(=O)NC4=O)c3)CC2)CC1 ; CN(CCC(=O)N1CCC([C@@H]2CCNc3c(C(N)=O)c(-c4ccc(Oc5ccccc5)cc4)nn32)CC1)CC1CCC(N2CCN(c3cccc(N4CCC(=O)NC4=O)c3)CC2)CC1 ; NC(=O)c1c(-c2ccc(Oc3ccccc3)cc2)nn2c1NCC[C@H]2C1CCN(C(=O)CC2CCC(N3CCN(c4ccc5c(c4)C(=O)N(C4CCC(=O)NC4=O)C5=O)CC3)CC2)CC1 ; NC(=O)c1c(-c2ccc(Oc3ccccc3)cc2)nn2c1NCC[C@H]2C1CCN(C(=O)CCCC2CCC(N3CCN(c4ccc5c(c4)C(=O)N(C4CCC(=O)NC4=O)C5=O)CC3)CC2)CC1]

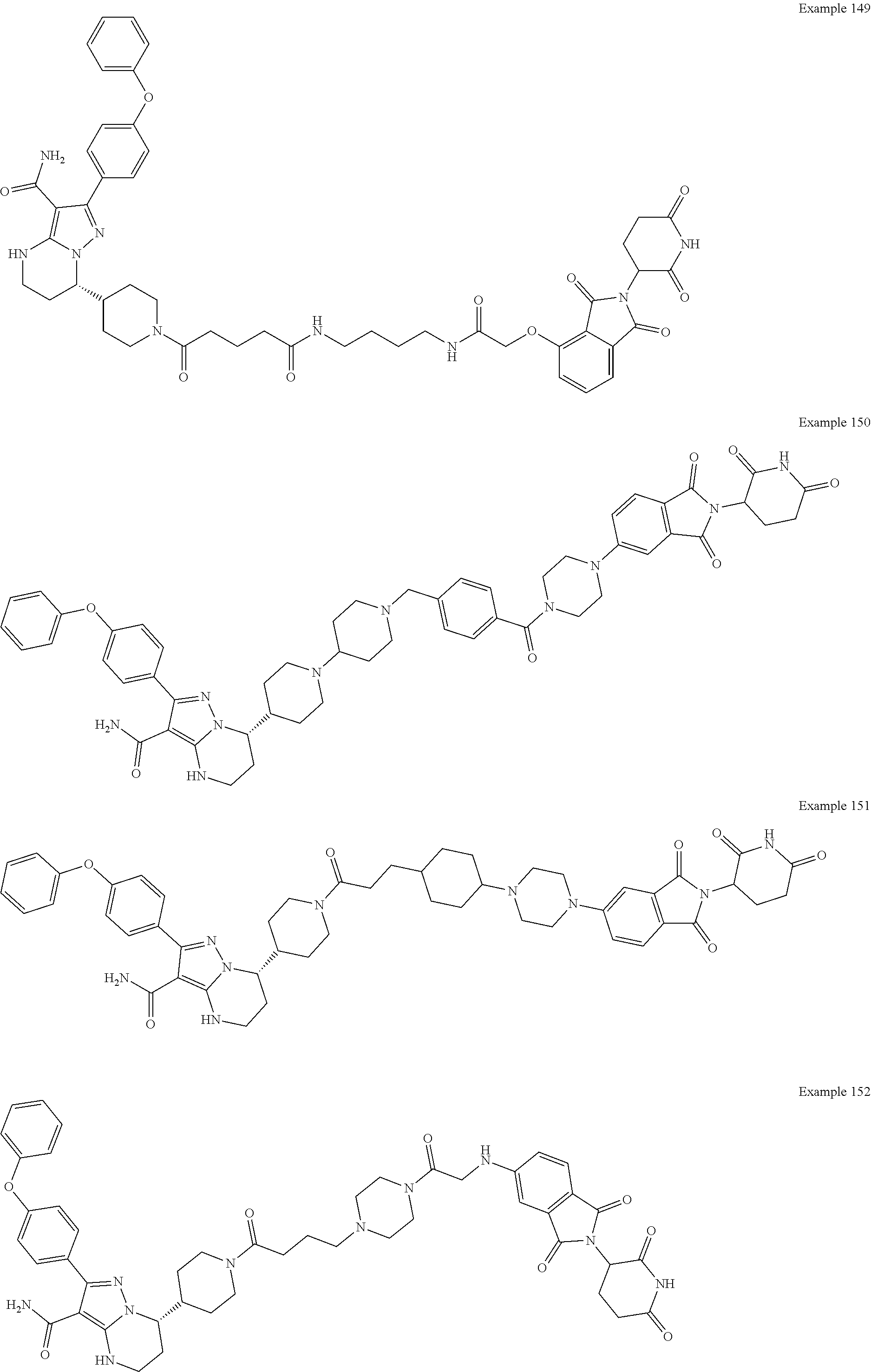 [SMILES: NC(=O)c1c(-c2ccc(Oc3ccccc3)cc2)nn2c1NCC[C@H]2C1CCN(C(=O)CCCC(=O)NCCCCNC(=O)COc2cccc3c2C(=O)N(C2CCC(=O)NC2=O)C3=O)CC1 ; NC(=O)c1c(-c2ccc(Oc3ccccc3)cc2)nn2c1NCC[C@H]2C1CCN(C2CCN(Cc3ccc(C(=O)N4CCN(c5ccc6c(c5)C(=O)N(C5CCC(=O)NC5=O)C6=O)CC4)cc3)CC2)CC1 ; NC(=O)c1c(-c2ccc(Oc3ccccc3)cc2)nn2c1NCC[C@H]2C1CCN(C(=O)CCC2CCC(N3CCN(c4ccc5c(c4)C(=O)N(C4CCC(=O)NC4=O)C5=O)CC3)CC2)CC1 ; NC(=O)c1c(-c2ccc(Oc3ccccc3)cc2)nn2c1NCC[C@H]2C1CCN(C(=O)CCCN2CCN(C(=O)CNc3ccc4c(c3)C(=O)N(C3CCC(=O)NC3=O)C4=O)CC2)CC1]

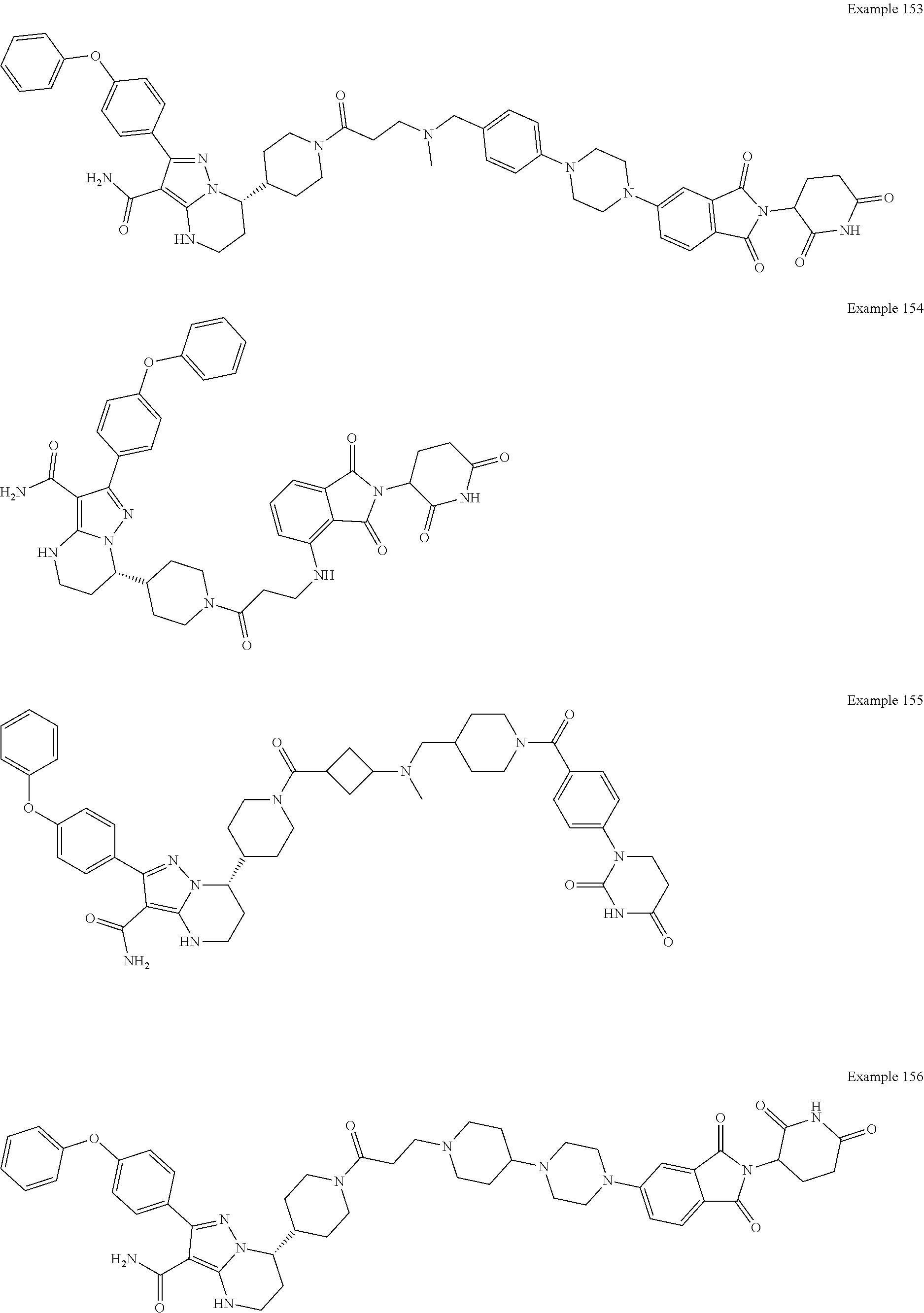 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)N(C3CCC(=O)NC3=O)C4=O)CC2)cc1 ; NC(=O)c1c(-c2ccc(Oc3ccccc3)cc2)nn2c1NCC[C@H]2C1CCN(C(=O)CCNc2cccc3c2C(=O)N(C2CCC(=O)NC2=O)C3=O)CC1 ; CN(CC1CCN(C(=O)c2ccc(N3CCC(=O)NC3=O)cc2)CC1)C1CC(C(=O)N2CCC([C@@H]3CCNc4c(C(N)=O)c(-c5ccc(Oc6ccccc6)cc5)nn43)CC2)C1 ; NC(=O)c1c(-c2ccc(Oc3ccccc3)cc2)nn2c1NCC[C@H]2C1CCN(C(=O)CCN2CCC(N3CCN(c4ccc5c(c4)C(=O)N(C4CCC(=O)NC4=O)C5=O)CC3)CC2)CC1]

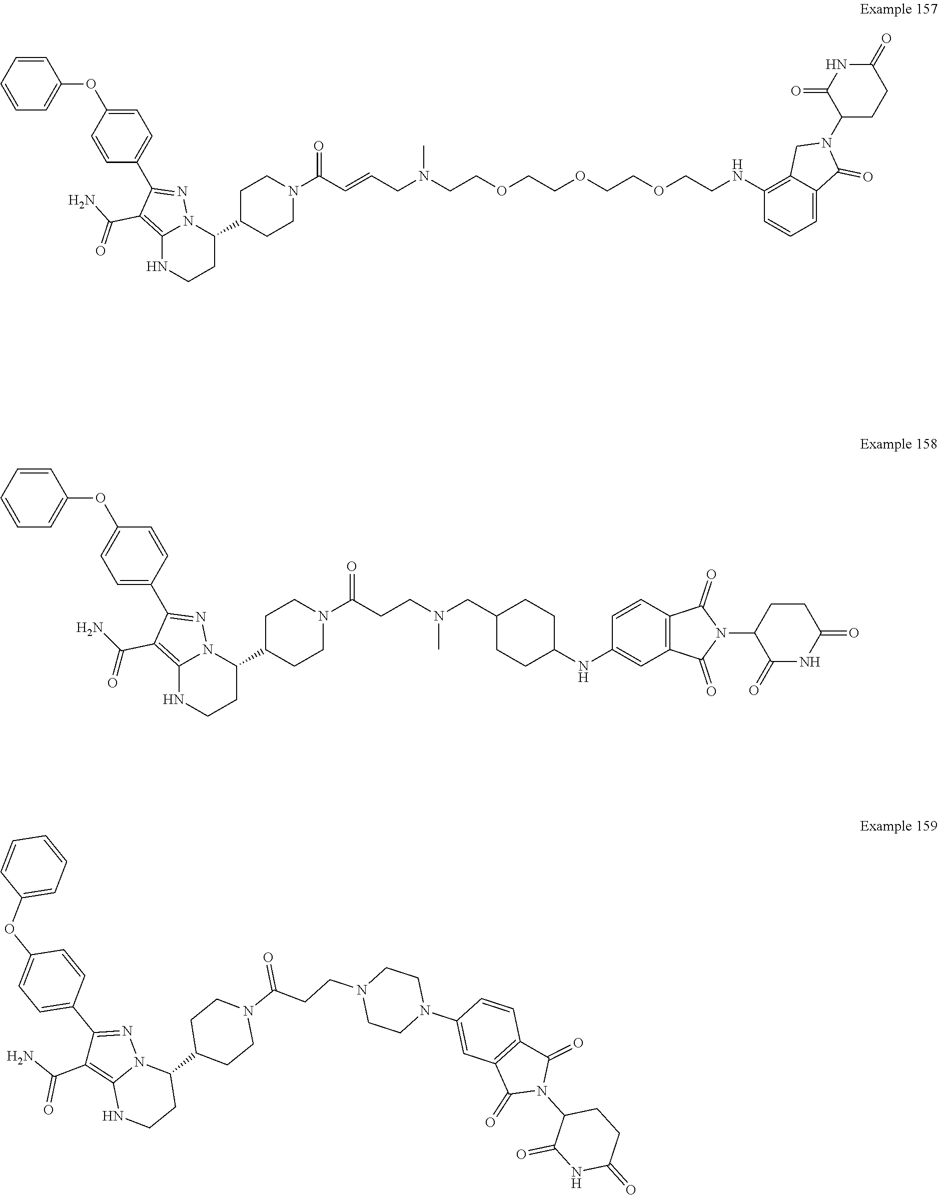 [SMILES: CN(C/C=C/C(=O)N1CCC([C@@H]2CCNc3c(C(N)=O)c(-c4ccc(Oc5ccccc5)cc4)nn32)CC1)CCOCCOCCOCCNc1cccc2c1CN(C1CCC(=O)NC1=O)C2=O ; CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1 ; NC(=O)c1c(-c2ccc(Oc3ccccc3)cc2)nn2c1NCC[C@H]2C1CCN(C(=O)CCN2CCN(c3ccc4c(c3)C(=O)N(C3CCC(=O)NC3=O)C4=O)CC2)CC1]

In the second aspect, disclosed herein is a pharmaceutical composition comprising the compound disclosed herein, or a pharmaceutically acceptable salt thereof, and at least one pharmaceutically acceptable carrier or excipient.

In the third aspect, disclosed herein is a method of inhibiting BTK activity, which comprises administering to an individual the compound disclosed herein, or a pharmaceutically acceptable salt thereof, including the compound of formula (I) or the specific compounds exemplified herein.

In the fourth aspect, disclosed herein is a method of treating a disease or disorder in a patient comprising administering to the patient a therapeutically effective amount of the compound disclosed herein, or a pharmaceutically acceptable salt thereof as an BTK kinase inhibitor, wherein the compound disclosed herein includes the compound of formula (I) or the specific compounds exemplified herein. In some embodiments, the disease or disorder is associated with inhibition of BTK. Preferably, the disease or disorder is cancer.

### DETAILED DESCRIPTION OF THE INVENTION

### Definitions

The following terms have the indicated meaning throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” refers to a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include without limitation to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (Cl), bromo (Br) and iodo (I).

The term “haloalkyl” refers to an alkyl group in which one or more hydrogens are replaced by one or more halogen atoms such as fluoro, chloro, bromo, and iodo. Examples of the haloalkyl include without limitation to haloC₁₋₈alkyl, haloC₁₋₆ alkyl or halo C₁₋₄ alkyl, such as —CF₃, —CH₂C₁, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include without limitation to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include without limitation to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈ cycloalkyl, include without limitation to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to cyclopropyl, cyclobutyl, cyclopentyl, and cyclohexyl. Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, preferably cyclohexenyl.

The term “fused cycloalkenyl” refers to a bicyclic cycloalkyl group as defined herein which contain at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond.

The term “fused cycloalkynyl” refers to a bicyclic cycloalkyl group as defined herein which contains at least one triple bond and is formed by two or more rings sharing two adjacent atoms.

The term a “benzo fused cycloalkyl” is a bicyclic fused cycloalkyl in which a 4- to 8-membered monocyclic cycloalkyl ring fused to a benzene ring. For example, a benzo fused cycloalkyl is

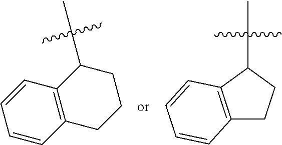

wherein the wavy lines indicate the points of attachment.

The term a “benzo fused cycloalkenyl” is a bicyclic fused cycloalkenyl in which a 4- to 8-membered monocyclic cycloalkenyl ring fused to a benzene ring.

The term a “benzo fused cycloalkynyl” is a bicyclic fused cycloalkynyl in which a 4- to 8-membered monocyclic cycloalkynyl ring fused to a benzene ring.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2, 3-dihydro-1H-indenyl, 1H-indenyl, 1, 2, 3, 4-tetralyl, 1, 4-dihydronaphthyl, etc. Preferred embodiments are 8 to 9 membered fused rings, which refer to cyclic structures containing 8 to 9 ring atoms within the above examples.

The term “aryl” used alone or in combination with other terms refers to a group selected from:
a) 5- and 6-membered carbocyclic aromatic rings, e.g., phenyl;
b) bicyclic ring systems such as 7 to 12 membered bicyclic ring systems, wherein at least one ring is carbocyclic and aromatic, e.g., naphthyl and indanyl; and,
c) tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include without limitation to phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene.

The term “heteroaryl” refers to a group selected from:
a) 5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
b) 7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
c) 11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

Representative examples of bicyclic fused heteroaryl include without limitation to the following groups: benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur” used herein refer to S, SO or SO₂.

The term “monocyclic heterocyclyl” refers to monocyclic groups in which at least one ring member (e.g., 1-3 heteroatoms, 1 or 2 heteroatom(s)) is a heteroatom selected from nitrogen, oxygen or optionally oxidized sulfur. A heterocycle may be saturated or partially saturated.

Exemplary monocyclic 4 to 9-membered heterocyclyl groups include without limitation to pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1, 2-dithietanyl, 1, 3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1, 4-oxathianyl, 1, 4-dioxepanyl, 1, 4-oxathiepanyl, 1, 4-oxaazepanyl, 1, 4-dithiepanyl, 1, 4-thiazepanyl and 1, 4-diazepanyl, 1, 4-dithianyl, 1, 4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl could be mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered, 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include without limitation to the following groups: 2, 3-dihydrospiro[indene-1, 2′-pyrrolidine] (e.g., 2, 3-dihydrospiro[indene-1, 2′-pyrrolidine]-1′-yl), 1, 3-dihydrospiro[indene-2, 2′-pyrrolidine] (e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3]heptane (e.g., 2-oxa-6-azaspiro[3.3]heptan-6-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1, 7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl could be bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include without limitation to the following groups: octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, or tetrahydropyrazolopyrimidinyl (e.g., 4, 5, 6, 7-tetrahydropyrazolo[1,5-a]pyrimidin-3-yl).

The term a “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl could be bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include without limitation to the following groups: 2-azabicyclo[2.2.1]heptyl, azabicyclo[3.1.0]hexyl, 2-azabicyclo[2.2.2]octyl and 2-azabicyclo[3.3.2]decyl.

The term “at least one substituent” disclosed herein includes, for example, from 1 to 4, such as from 1 to 3, further as 1 or 2, substituents, provided that the theory of valence is met. For example, “at least one substituent R^{6d}” disclosed herein includes from 1 to 4, such as from 1 to 3, further as 1 or 2, substituents selected from the list of R^{6d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical or chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers and diastereomers can also be separated by the use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W, Ed. Drug Stereochemistry: Analytical Methods and Pharmacology. New York: Marcel Dekker, Inc., 1993.

“Pharmaceutically acceptable salts” refer to those salts which are, within the scope of sound medical judgment, suitable for use in contact with the tissues of humans and animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic agent, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical route to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc., a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, an aromatic, a sweetener, a dye and etc.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### General Reaction Scheme for Compound Preparation

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of desired product. Some of compounds in this invention may be generated by the methods as shown in the following reaction schemes and the description thereof.

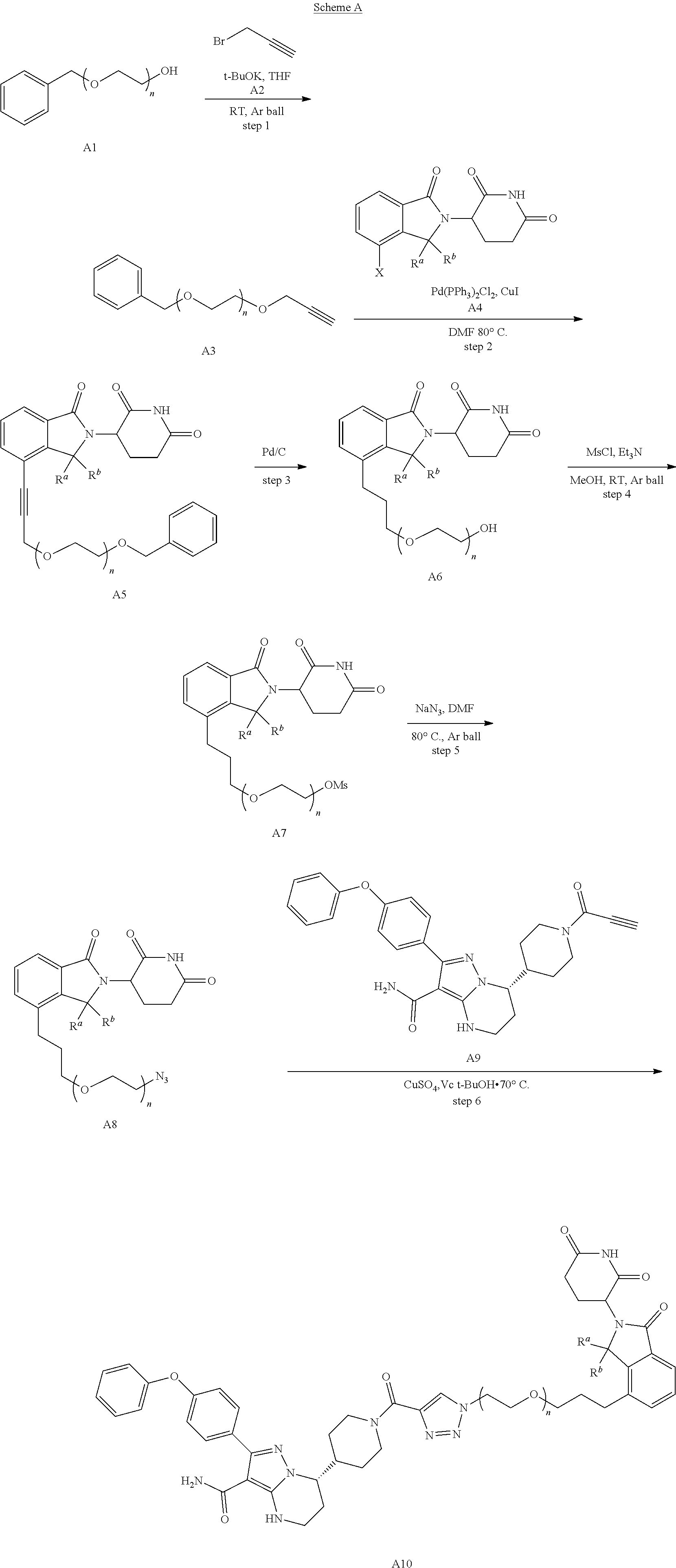

Wherein R^{a}, R^{b}, X and n are defined as described herein. A3 can be synthesized from A1 and A2 in the basic condition, then A3 coupled with A4 with basic condition or metal as catalysis to form A5. Benzyl group was removed in Pd/C condition to give A6, which was converted to A7 with MsCl. A8 was synthesized from A7 and NaN₃ by heated in DMF, which coupled with A9 to give A10 in the present of CuSO₄ and Vc condition.

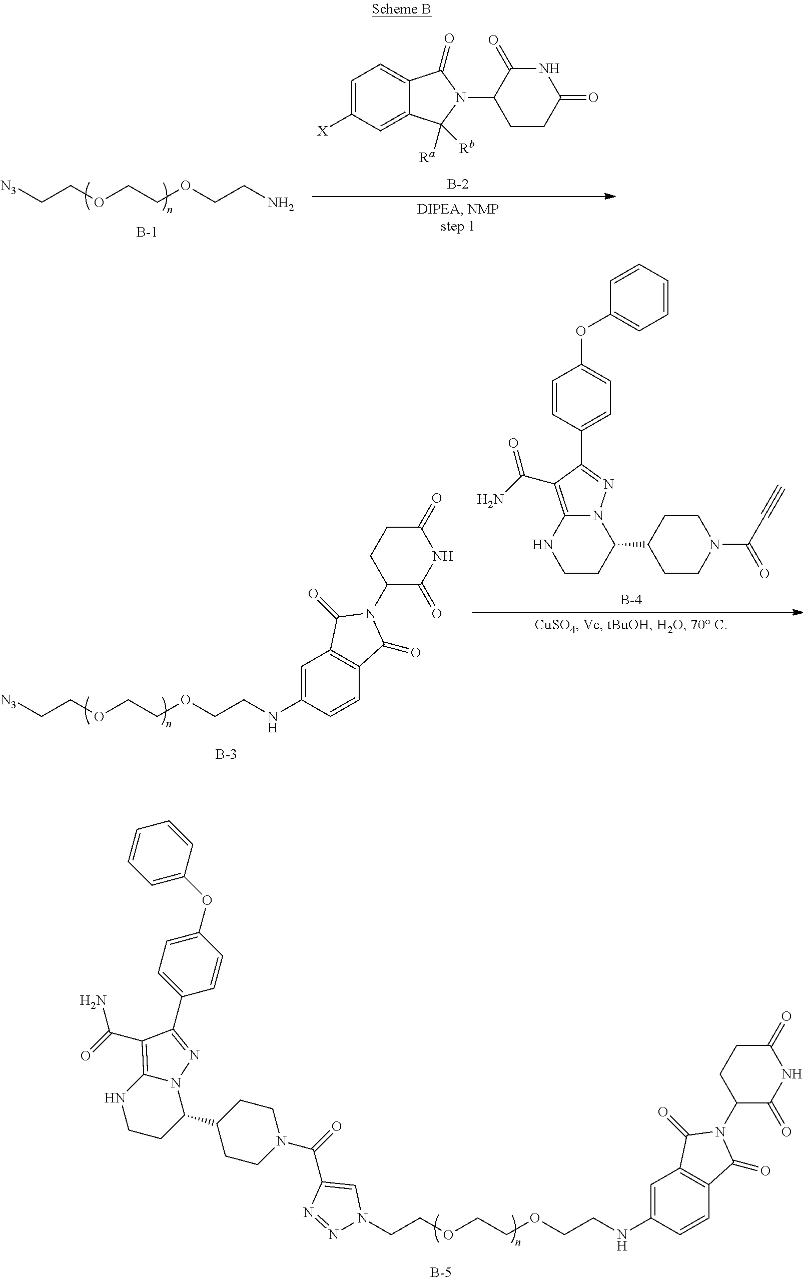

Wherein R^{a}, R^{b}, X and n are defined as described herein. B-3 can be synthesized from B-1 and B-2 in basic condition, and then B-3 can be converted to B-5 in the presence of CuSO₄ and Vc.

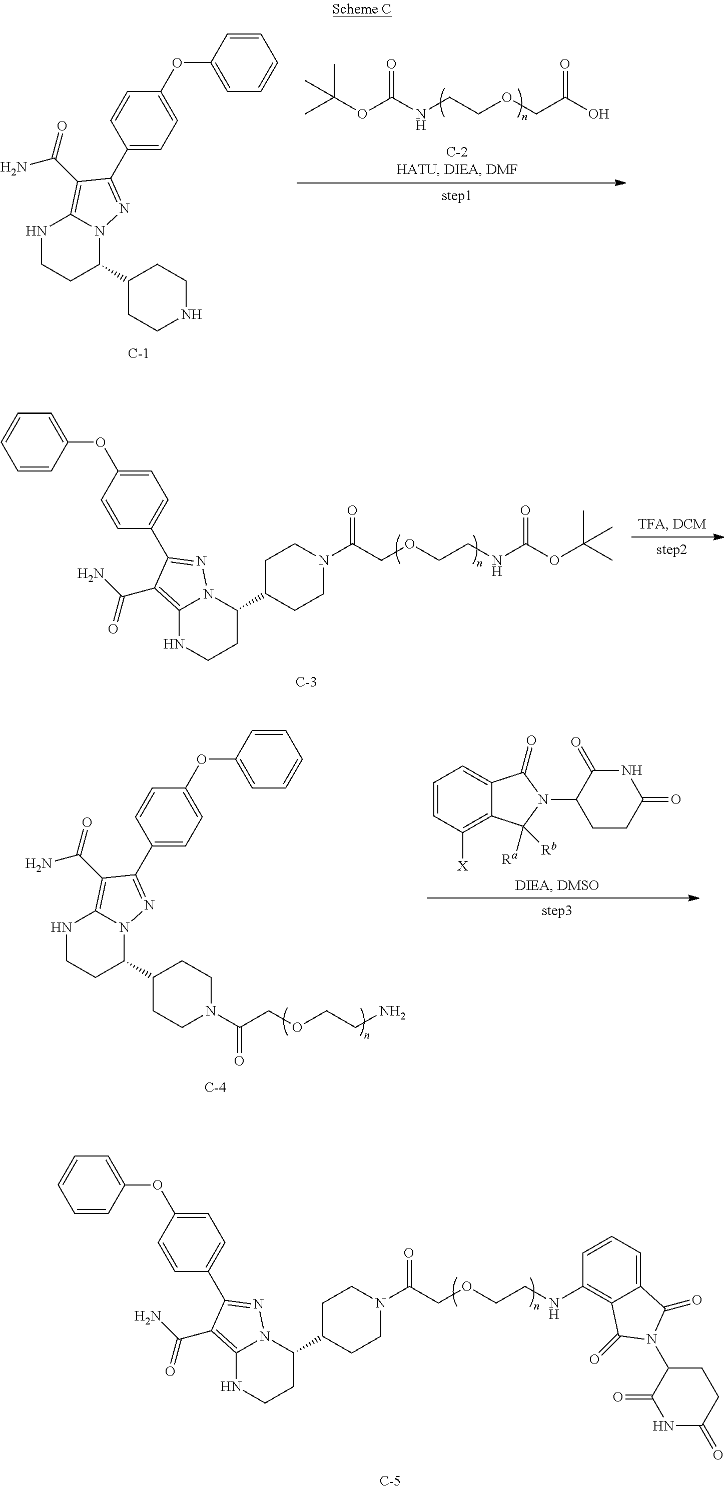

Wherein R^{a}, R^{b}, X and n are defined as described herein. C-3 can be synthesized from C-1 and C-2 in coupling reagent (for example: HATU, PyBOP, EDCI, HOBt), and then the Boc group in C-3 was deported by CF₃COOH to give C-4, which can be converted to C-5 in the basic condition by heated.

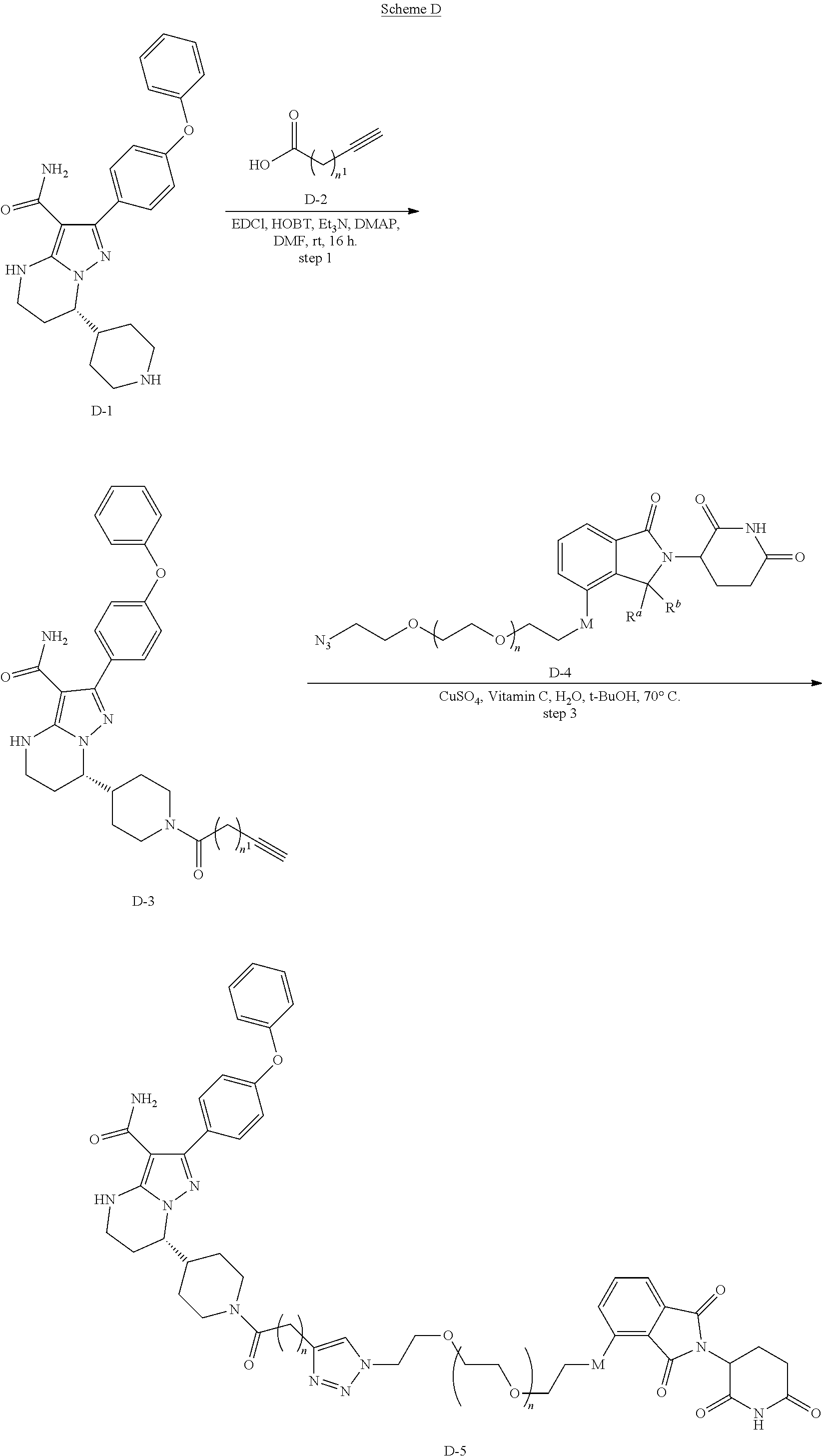

Wherein R^{a}, R^{b}, M, n and n¹ are defined as described herein. D-3 can be synthesized from D-1 and D-2 in coupling reagent (for example: HATU, PyBOP, EDCI, HOBt), and then D-3 can be converted to D-5 in the present of CuSO₄ and Vc.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC-MS spectrometer (Agilent 1260) Detector: MWD (190-400 nm), Mass detector: 6120 SQ Mobile phase: A: acetonitrile with 0.1% Formic acid, B: water with 0.1% Formic acid Column: Poroshell 120 EC-C18, 4.6×50 mm, 2.7 pm Gradient method: Flow: 1.8 mL/min Time (min) A (%) B (%)

| Time (min) | A (%) | B (%) |
|---|---|---|
| 0.00 | 5 | 95 |
| 1.5 | 95 | 5 |
| 2.0 | 95 | 5 |
| 2.1 | 5 | 95 |
| 3.0 | 5 | 95 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| AcOH | Acetic acid |
| Aq | Aqueous |
| Brine | Saturated aqueous sodium chloride solution |
| Bn | Benzyl |
| BnBr | Benzyl Bromide |
| Boc | Tert-butyloxycarbonyl |
| Cbz | benzyloxycarbonyl |
| CH₂Cl₂ | Dichloromethane |
| DMF | N,N-Dimethylformamide |
| Dppf | 1,1″-bis(diphenylphosphino)ferrocene |
| DBU | 1,8-diazabicyclo[5.4.0]undec-7-ene |
| DCM | Dichloromethane |
| DIEA or | N,N-diisopropylethylamine |
| DIPEA | |
| DIBAL-H | Diisobutylaluminium hydride |
| DMAP | 4-N,N-dimethylaminopyridine |
| DMF | N,N-dimethylformamide |
| DMSO | Dimethyl sulfoxide |
| EA or EtOAc | Ethyl acetate |
| EtOH | Ethanol |
| Et₂O or ether | Diethyl ether |
| g | Grams |
| h or hr | Hour |
| HATU | O-(7-Azabenzotriazol-1-yl)-N,N,N′,N′-tetramethyluronium |
| | hexafluorophosphate |
| HBTU | O-(7-Azabenzotriazol-1-yl)-N,N,N′,N′-tetramethyluronium |
| | hexafluorophosphate |
| HCl | Hydrochloric acid |
| Hex | Hexane |
| HPLC | High-performance liquid chromatography |
| IPA | 2-propanol |
| i-PrOH | Isopropyl alcohol |
| mg | Milligrams |
| mL | Milliliters |
| Mmol | Millimole |
| MeCN | Acetonitrile |
| MeOH | Methanol |
| Min | Minutes |
| ms or MS | Mass spectrum |
| Na₂SO₄ | Sodium sulfate |
| PE | Petroleum ether |
| Ph₃PO | Triphenyl phosphorus oxide |
| PPA | Polyphosphoric acid |
| Rt | Retention time |
| R.T. or r.t. | Room temperature |
| TBAF | Tetra-butyl ammonium fluoride |
| TBSCl | tert-Butyldimethylsilyl chloride |
| TEA | Triethanolamine |
| TFA | Trifluoroacetic acid |
| Tf₂O | Triflic anhydride |
| THF | Tetrahydrofuran |
| TLC | thin layer chromatography |
| Ts | para-toluenesulfonyl |
| TBS | tert-butyldimethylsilyl |
| μL | Microliters |

### Example 1: (7S)-7-(1-(1-(2-(3-(2-(2, 6-dioxopiperidin-3-yl)-1-oxoisoindolin-4-yl)propoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

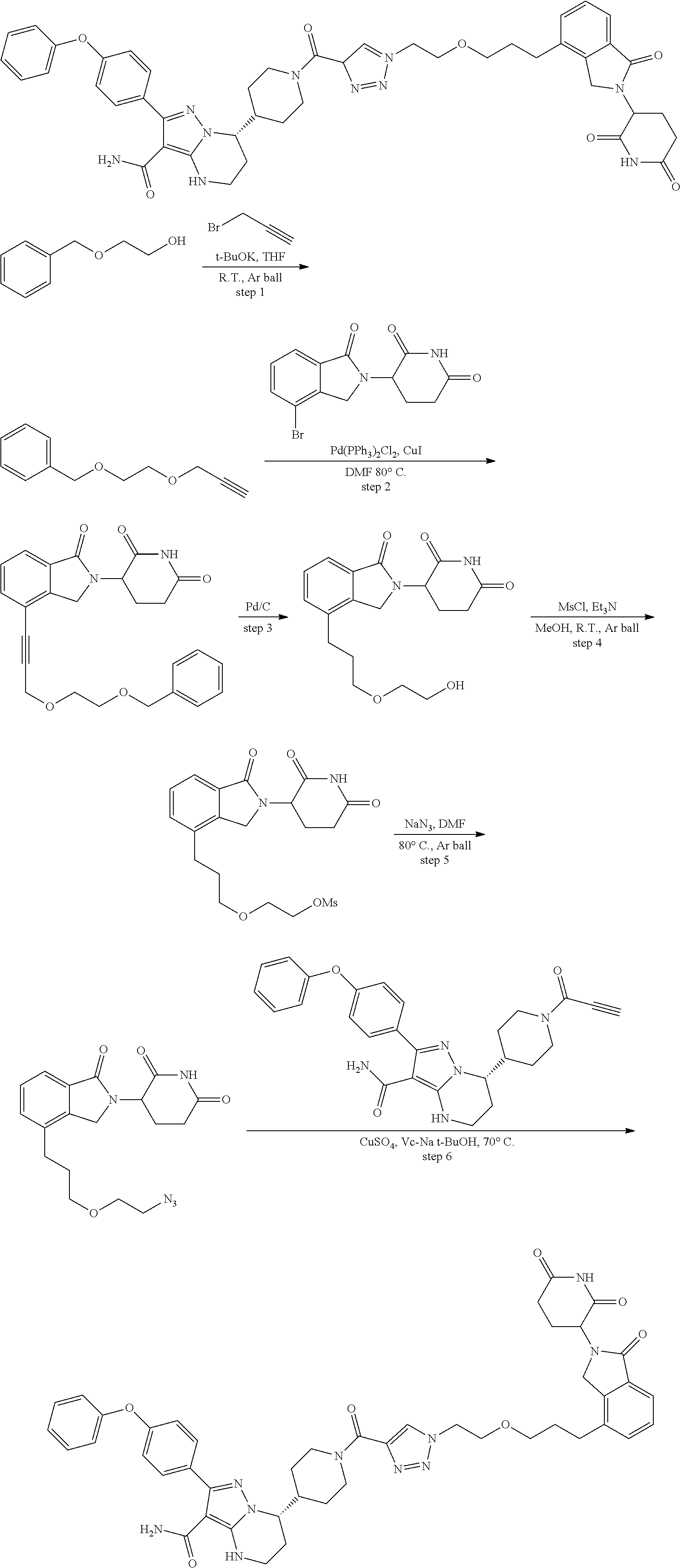

### Step 1: ((2-(prop-2-yn-1-yloxy)ethoxy)methyl)benzene

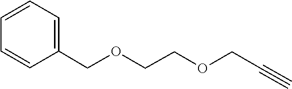

A mixture of 2-(benzyloxy)ethan-1-ol (10 g, 65.8 mmol) and t-BuOK (3.9 g, 34.87 mmol) in dry THF (100 mL) was stirred in a round bottom flask at RT for 20 min under the atmosphere of Ar. Then, 3-bromoprop-1-yne (8.08 g, 69.1 mmol) was added dropwise. The mixture was stirred at R.T. for 12 h under the atmosphere of Ar. The reaction was monitored with TLC (stained by KMnO₄). When the reaction was completed, solvent was evaporated in vacuum and saturated aqueous NaCl was added to quench the reaction. The mixture was acidified with 1M HCl to pH=3-5, then extracted with DCM. The organic layer was dried over anhydrous Na₂SO₄, and evaporated in vacuum to afford the crude product, which was further purified with silica gel column chromatography (petroleum ether:ethyl acetate=6:1) to give the product (8 g, 70%).

### Step 2: 3-(4-(3-(2-(benzyloxy)ethoxy)prop-1-yn-1-yl)-1-oxoisoindolin-2-yl)piperidine-2, 6-dione

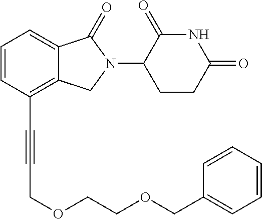 [SMILES: O=C1CCC(N2Cc3c(C#CCOCCOCc4ccccc4)cccc3C2=O)C(=O)N1]

A mixture of ((2-(prop-2-yn-1-yloxy)ethoxy)methyl)benzene (0.4 g, 1.24 mmol), 3-(4-bromo-1-oxoisoindolin-2-yl)piperidine-2, 6-dione (0.59 g, 3.1 mmol), bis(triphenylphosphine) palladium (II) chloride (87 mg, 0.124 mmol) and CuI (47 mg, 0.248 mmol) in dry DMF (10 mL) was stirred in a round bottom flask at R.T. for 2 min under the atmosphere of Ar. Then, dry Et₃N (10 mL) was added. The mixture was stirred at 80° C. for 3 h under the atmosphere of Ar. The reaction was monitored with TLC. When the reaction was completed, the solvent was evaporated in vacuum. The crude product thus obtained was further purified with silica gel column chromatography (DCM:MeOH=50: 1-10:1 gradient elution) to give the product (0.5 g, 94%). [M+H]⁺=433.0.

### Step 3: 3-(4-(3-(2-hydroxyethoxy)propyl)-1-oxoisoindolin-2-yl)piperidine-2, 6-dione

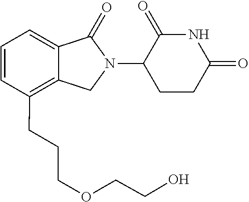 [SMILES: O=C1CCC(N2Cc3c(CCCOCCO)cccc3C2=O)C(=O)N1]

A mixture of 3-(4-(3-(2-(benzyloxy)ethoxy)prop-1-yn-1-yl)-1-oxoisoindolin-2-yl)piperidine-2, 6-dione (0.5 g, 1.16 mmol) in MeOH (10 mL) and DMF (10 mL) was stirred in a round bottom flask for 2 min at R.T. under the atmosphere of Ar. Pd/C was added. Ar in bottom was replaced by H₂. The mixture was stirred at 38° C. for 12 h under H₂ at a pressure of 50 psi. The reaction was monitored with TLC. When the reaction was completed, black solid was removed from the solution via filtration. The solvent was evaporated in vacuum to afford the crude product, which was further purified with silica gel column chromatography (DCM:MeOH=40:1˜15:1 gradient elution) to give the product (10 mg, 2.5%). [M+H]⁺=347.0.

### Step 4: 2-(3-(2-(2, 6-dioxopiperidin-3-yl)-1-oxoisoindolin-4-yl)propoxy)ethyl methanesulfonate

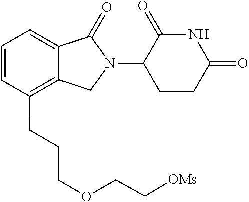 [SMILES: CS(=O)(=O)OCCOCCCc1cccc2c1CN(C1CCC(=O)NC1=O)C2=O]

A mixture of 3-(4-(3-(2-hydroxyethoxy)propyl)-1-oxoisoindolin-2-yl)piperidine-2, 6-dione (6 mg, 0.0173 mmol) and Et₃N (5.2 mg, 0.0519 mmol) in dry THF (10 mL) was stirred in a round bottom flask at 0° C. for 5 min under the atmosphere of Ar. Then, MSCl (2.6 mg, 0.0225 mmol) was added. The mixture was warmed to R.T. and stirred for 4 h under the atmosphere of Ar. The reaction was monitored with TLC. When the reaction was completed, the solvent was evaporated in vacuum. The crude product thus obtained was further purified with pre-TLC (DCM:MeOH=50:1) to give the product (8 mg, 90%). [M+H]⁺=425.0.

### Step 5: 3-(4-(3-(2-azidoethoxy)propyl)-1-oxoisoindolin-2-yl)piperidine-2, 6-dione

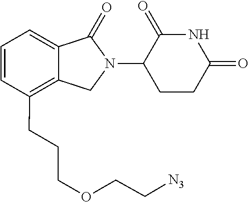 [SMILES: [N-]=[N+]=NCCOCCCc1cccc2c1CN(C1CCC(=O)NC1=O)C2=O]

A mixture of 2-(3-(2-(2, 6-dioxopiperidin-3-yl)-1-oxoisoindolin-4-yl)propoxy)ethyl methanesulfonate (8 mg, 0.019 mmol) and NaN₃ (2.5 mg, 0.038 mmol) in dry DMF (6 mL) was stirred in a round bottom flask at 80° C. for 4 h under the atmosphere of Ar. The reaction was monitored with TLC. When the reaction was completed, saturated aqueous NaCl was added to quench the reaction and extracted with EtOAc. The organic layer was dried over anhydrous Na₂SO₄ and evaporated in vacuum to afford the crude product, which was further purified with pre-TLC (DCM:MeOH=50:1) to give the product (10 mg, 90%). [M+H]⁺=372.0.

### Step 6: (7S)-7-(1-(1-(2-(3-(2-(2, 6-dioxopiperidin-3-yl)-1-oxoisoindolin-4-yl)propoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

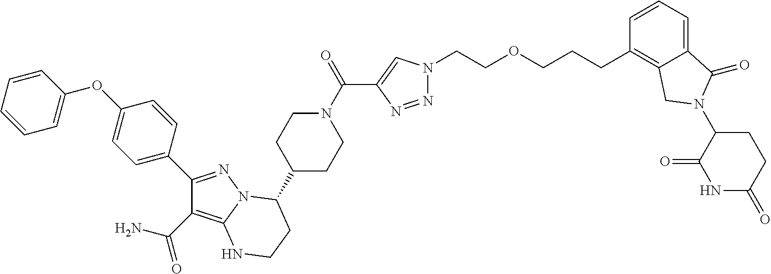 [SMILES: NC(=O)c1c(-c2ccc(Oc3ccccc3)cc2)nn2c1NCC[C@H]2C1CCN(C(=O)c2cn(CCOCCCc3cccc4c3CN(C3CCC(=O)NC3=O)C4=O)nn2)CC1]

A mixture of 3-(4-(3-(2-azidoethoxy)propyl)-1-oxoisoindolin-2-yl)piperidine-2, 6-dione (10 mg, 0.027 mmol), (S)-2-(4-phenoxyphenyl)-7-(1-propioloylpiperidin-4-yl)-4, 5, 6, 7-tetrahydropyrazolo[1,5-a]pyrimidine-3-carboxamide (15.2 mg, 0.0323 mmol), CuSO₄ (10.8 mg, 0.0432 mmol), sodium ascorbate (15.7 mg, 0.089 mmol) and H₂O (0.5 mL) in t-BuOH (5 mL) was stirred in a round bottom flask for 7 h at 70° C. under the atmosphere of Ar. The reaction was quenched with saturated aqueous NaHCO₃ and extracted with DCM. The organic layer was dried over anhydrous Na₂SO₄, and evaporated in vacuum to afford the crude product, which was further purified with pre-TLC (DCM:MeOH=20:1) to give the product (2.3 mg, 10.5%). ¹H NMR (400 MHz, DMSO) δ_{H} 10.99 (s, 1H), 8.49 (s, 1H), 7.58-7.32 (m, 7H), 7.17 (t, J=7.2 Hz, 1H), 7.07 (dd, J=12.0, 8.4 Hz, 4H), 6.67 (s, 1H), 5.12 (dd, J=12.8, 4.4 Hz, 1H), 4.80 (s, 1H), 4.70-4.49 (m, 4H), 4.34 (dd, J=60.8, 17.2 Hz, 3H), 4.03-3.95 (m, 1H), 3.83-3.77 (m, 2H), 3.44-3.35 (m, 3H), 3.15-2.82 (m, 6H), 2.75-2.60 (m, 5H), 2.07-1.71 (m, 10H), 1.67-1.40 (m, 5H); [M+H]⁺=841.0.

### Example 2: (7S)-7-(1-(1-(2-(2-((2-(2, 6-dioxopiperidin-3-yl)-1, 3-dioxoisoindolin-4-yl)amino)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

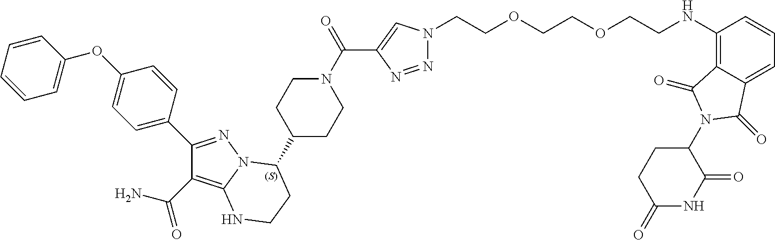

The titled compound was synthesized in the procedures similar to Example 1. ¹H NMR (400 MHz, CDCl₃) δ_{H} 8.67-8.26 (m, 1H), 8.19 (s, 1H), 7.59 (d, J=7.4 Hz, 1H), 7.51 (d, J=7.4 Hz, 2H), 7.37 (t, J=7.7 Hz, 2H), 7.19-6.93 (m, 6H), 6.78 (s, 1H), 6.65 (brs, 1H), 5.42-5.05 (m, 3H), 4.93-4.82 (m, 2H), 4.58 (s, 2H), 4.14 (s, 1H), 3.88 (s, 2H), 3.76-3.27 (m, 6H), 3.16 (d, J=10.9 Hz, 1H), 2.96-2.42 (m, 5H), 2.13-2.01 (m, 3H), 1.97-1.73 (m, 3H), 1.46-1.25 (m, 2H); [M+H]⁺=855.8.

### Example 3: (7S)-7-(1-(1-(2-(2-(2-((2-(2, 6-dioxopiperidin-3-yl)-1, 3-dioxoisoindolin-4-yl)amino)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

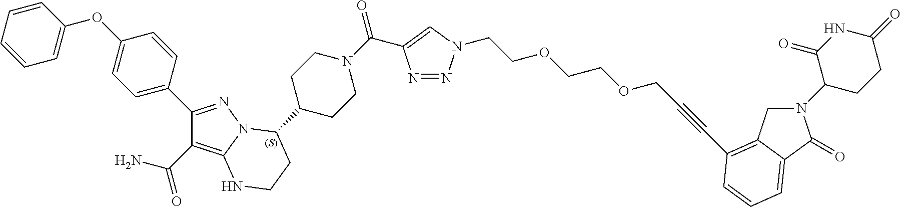

The titled compound was synthesized in the procedures similar to Example 1. ¹H NMR (400 MHz, DMSO) δ_{H} 11.09 (s, 1H), 8.45 (s, 1H), 7.49-7.58 (m, 3H), 7.39-7.43 (m, 2H), 7.02-7.19 (m, 6H), 6.68 (s, 1H), 6.59 (t, J=4.0 Hz, 1H), 5.02-5.08 (m, 1H), 4.83-4.87 (m, 1H), 4.50-4.57 (m, 3H), 3.99-4.04 (m, 1H), 3.84 (t, J=4.0 Hz, 2H), 3.53-3.58 (m, 6H), 3.41-3.45 (m, 2H), 3.26-3.31 (m, 2H), 3.03-3.13 (m, 1H), 2.82-2.91 (m, 1H), 2.53-2.73 (m, 3H), 2.27-2.35 (m, 1H), 1.85-2.05 (m, 3H), 1.73-1.78 (m, 1H), 1.58-1.64 (m, 1H), 1.25-1.40 (m, 2H); [M+H]⁺=899.8.

### Example 4: (7S)-7-(1-(1-(2-(2-((3-(2-(2, 6-dioxopiperidin-3-yl)-1-oxoisoindolin-4-yl)prop-2-yn-1-yl)oxy)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

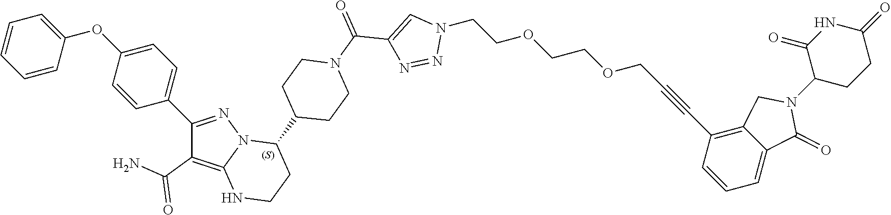

The titled compound was synthesized in the procedures similar to Example 1. ¹H NMR (400 MHz, DMSO) δ_{H} 11.00 (s, 1H), 8.47 (s, 1H), 7.73 (dd, J=21.2, 7.2 Hz, 2H), 7.59-7.47 (m, 3H), 7.41 (t, J=8.0 Hz, 2H), 7.17 (t, J=7.2 Hz, 1H), 7.07 (dd, J=10.4, 8.4 Hz, 4H), 6.68 (s, 1H), 5.14 (dd, J=13.2, 4.8 Hz, 1H), 4.83 (d, J=11.6 Hz, 1H), 4.62-4.55 (m, 2H), 4.54-4.44 (m, 2H), 4.43-4.28 (m, 3H), 4.01 (s, 1H), 3.86 (t, J=4.8 Hz, 2H), 3.61 (s, 4H), 3.30 (s, 3H), 3.10-3.00 (m, 1H), 2.98-2.84 (m, 2H), 2.70-2.56 (m, 3H), 2.44-2.31 (m, 3H), 2.09-1.84 (m, 4H), 1.82-1.50 (m, 2H), 1.44-1.24 (m, 3H); [M+H]⁺=880.9.

### Example 5: (7S)-7-(1-(1-(2-(2-(3-(2-(2, 6-dioxopiperidin-3-yl)-1-oxoisoindolin-4-yl)propoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

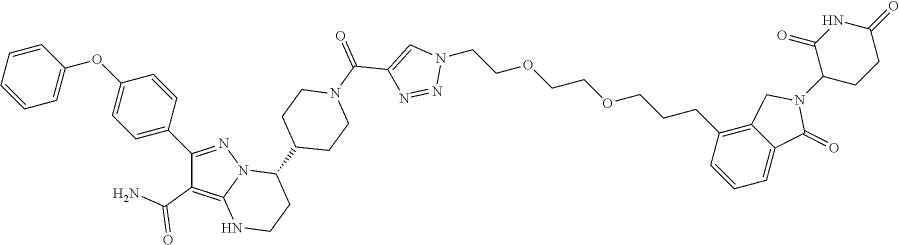 [SMILES: NC(=O)c1c(-c2ccc(Oc3ccccc3)cc2)nn2c1NCC[C@H]2C1CCN(C(=O)c2cn(CCOCCOCCCc3cccc4c3CN(C3CCC(=O)NC3=O)C4=O)nn2)CC1]

The titled compound was synthesized in the procedures similar to Example 1. ¹H NMR (400 MHz, DMSO) δ_{H} 10.99 (s, 1H), 8.46 (s, 1H), 7.61-7.36 (m, 8H), 7.17 (t, J=7.6 Hz, 1H), 7.06 (dd, J=11.6, 8.4 Hz, 4H), 6.68 (s, 1H), 5.12 (dd, J=12.8, 4.8 Hz, 1H), 4.83 (d, J=12.0 Hz, 1H), 4.62-4.23 (m, 6H), 4.05-3.94 (m, 1H), 3.84 (t, J=4.8 Hz, 1H), 3.57-3.40 (m, 6H), 3.13-2.91 (m, 3H), 2.69-2.58 (m, 4H), 2.44-2.20 (m, 3H), 2.05-1.50 (m, 9H), 1.43-1.13 (m, 4H); [M+H]⁺=884.9.

### Example 6: (7S)-7-(1-(1-(2-(2-(2-(3-(2-(2, 6-dioxopiperidin-3-yl)-1-oxoisoindolin-4-yl)propoxy)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

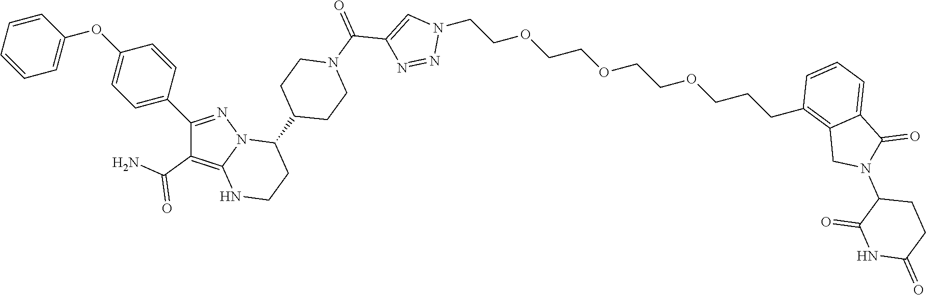 [SMILES: NC(=O)c1c(-c2ccc(Oc3ccccc3)cc2)nn2c1NCC[C@H]2C1CCN(C(=O)c2cn(CCOCCOCCOCCCc3cccc4c3CN(C3CCC(=O)NC3=O)C4=O)nn2)CC1]

The titled compound was synthesized in the procedures similar to Example 1. ¹H NMR (400 MHz, DMSO) δ_{H} 10.99 (s, 1H), 8.45 (s, 1H), 7.60-7.36 (m, 7H), 7.22-6.96 (m, 5H), 6.68 (s, 1H), 5.13 (dd, J=13.2, 4.8 Hz, 1H), 4.84 (d, J=12 Hz, 1H), 4.60-4.50 (m, 3H), 4.47-4.26 (m, 3H), 4.05-3.95 (m, 1H), 3.83 (t, J=4.8 Hz, 2H), 3.55-3.40 (m, 10H), 3.17-2.92 (m, 3H), 2.71-2.56 (m, 5H), 2.43-2.25 (m, 3H), 2.06-1.51 (m, 8H), 1.45-1.22 (m, 3H); [M+H]⁺=928.9.

### Example 7: (7S)-7-(1-(1-(2-(2-(2-((2-(2, 6-dioxopiperidin-3-yl)-1, 3-dioxoisoindolin-5-yl)amino)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

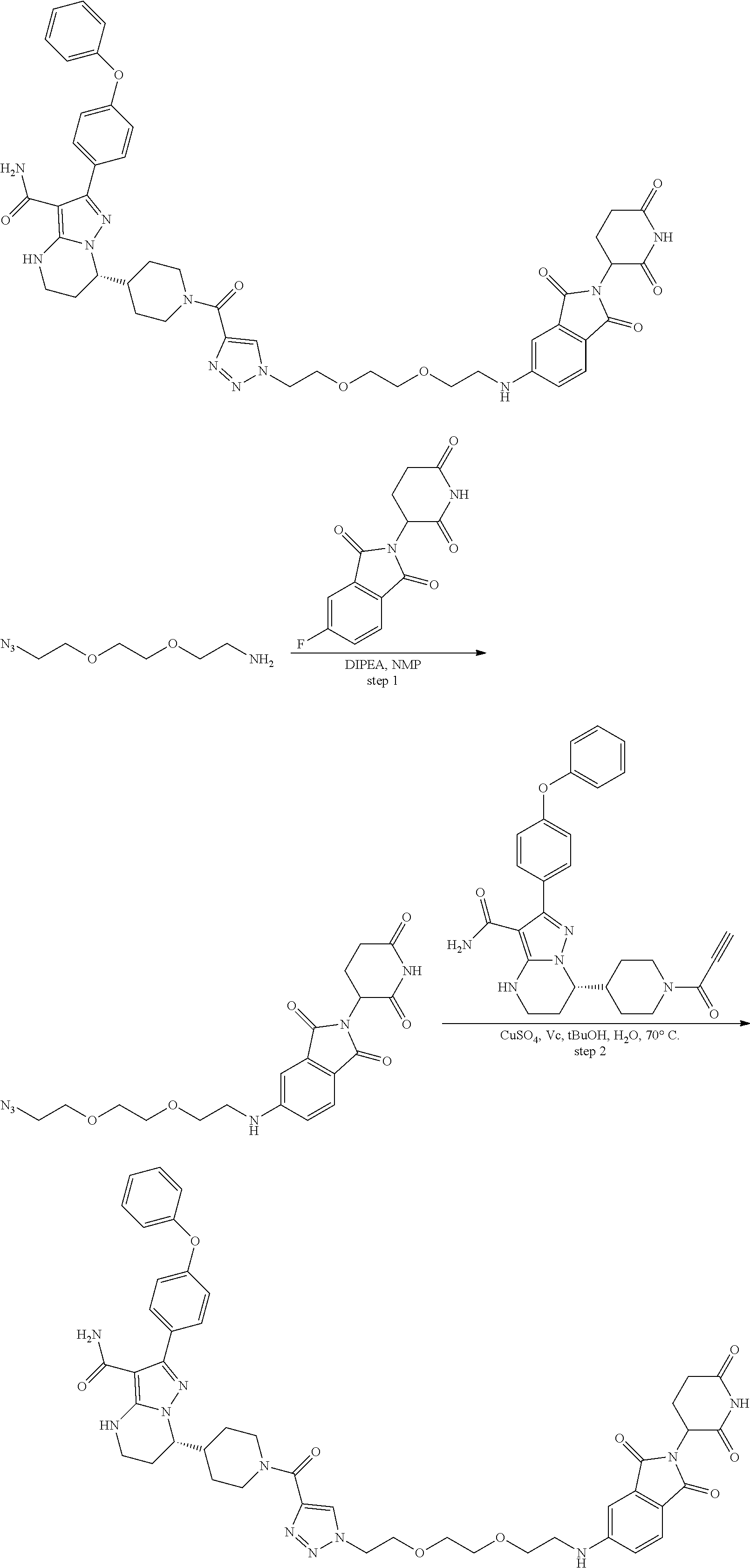 [SMILES: NC(=O)c1c(-c2ccc(Oc3ccccc3)cc2)nn2c1NCC[C@H]2C1CCN(C(=O)c2cn(CCOCCOCCNc3ccc4c(c3)C(=O)N(C3CCC(=O)NC3=O)C4=O)nn2)CC1 ; [N-]=[N+]=NCCOCCOCCN ; O=C1CCC(N2C(=O)c3ccc(F)cc3C2=O)C(=O)N1 ; [N-]=[N+]=NCCOCCOCCNc1ccc2c(c1)C(=O)N(C1CCC(=O)NC1=O)C2=O ; C#CC(=O)N1CCC([C@@H]2CCNc3c(C(N)=O)c(-c4ccc(Oc5ccccc5)cc4)nn32)CC1 ; NC(=O)c1c(-c2ccc(Oc3ccccc3)cc2)nn2c1NCC[C@H]2C1CCN(C(=O)c2cn(CCOCCOCCNc3ccc4c(c3)C(=O)N(C3CCC(=O)NC3=O)C4=O)nn2)CC1]

### Step 1: 5-((2-(2-(2-azidoethoxy)ethoxy)ethyl)amino)-2-(2, 6-dioxopiperidin-3-yl)isoindoline-1, 3-dione

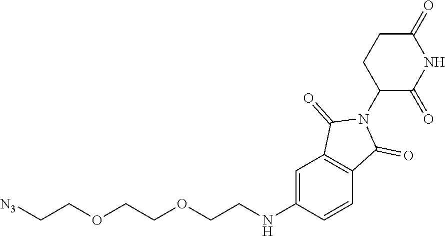 [SMILES: [N-]=[N+]=NCCOCCOCCNc1ccc2c(c1)C(=O)N(C1CCC(=O)NC1=O)C2=O]

A mixture of 2-(2-(2-azidoethoxy)ethoxy)ethan-1-amine (549 mg, 3.16 mmol, 1 eq), 2-(2, 6-dioxopiperidin-3-yl)-5-fluoroisoindoline-1, 3-dione (870.8 mg, 3.16 mmol, 1 eq), DIEA (0.92 mL, 5.06 mmol, 1.6 eq) in NMP (10 mL) was stirred in a round bottom flask at 90° C. overnight. The reaction was quenched with water and the mixture was washed once with saturated aqueous NaCl, then extracted with EtOAc. The organic layer was dried over anhydrous Na2SO₄, and evaporated in vacuum to afford the crude product, which was further purified with silica gel column chromatography (PE:EA=1: 2-1:10 gradient elution) to give the product (190 mg, 14%). [M+H]⁺=430.9.

### Step 2: (7S)-7-(1-(1-(2-(2-(2-((2-(2, 6-dioxopiperidin-3-yl)-1, 3-dioxoisoindolin-5-yl)amino)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

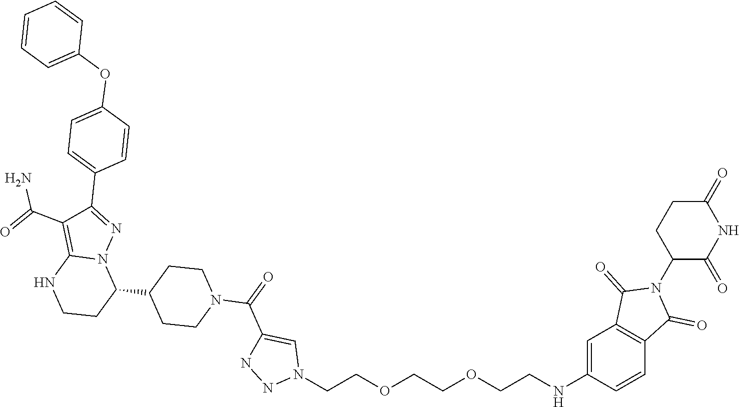 [SMILES: NC(=O)c1c(-c2ccc(Oc3ccccc3)cc2)nn2c1NCC[C@H]2C1CCN(C(=O)C2=CN(CCOCCOCCNc3ccc4c(c3)C(=O)N(C3CCC(=O)NC3=O)C4=O)NN2)CC1]

A mixture of 5-((2-(2-(2-azidoethoxy)ethoxy)ethyl)amino)-2-(2, 6-dioxopiperidin-3-yl)isoindoline-1, 3-dione (67 mg, 0.16 mmol), (S)-2-(4-phenoxyphenyl)-7-(1-propioloylpiperidin-4-yl)-4, 5, 6, 7-tetrahydropyrazolo[1,5-a]pyrimidine-3-carboxamide (73.2 mg, 0.16 mmol), CuSO4.5H2O (39 mg, 0.16 mmol), ascorbic acid (82.3 mg, 0.48 mmol), H₂O (0.5 mL) in t-BuOH (5 mL) was stirred in a round bottom flask for 5 h at 70° C. under the atmosphere of N₂. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (24.74 mg, 18%). 1H NMR (400 MHz, CDCl₃) δH 8.46-8.43 (m, 1H), 8.30-8.07 (m, 1H), 7.60-7.56 (m, 1H), 7.49-7.47 (m, 2H), 7.38 (t, J=8.0 Hz, 2H), 7.16 (t, J=7.2 Hz, 1H), 7.07-7.00 (m, 5H), 6.77 (t, J=8.4 Hz, 1H), 6.51 (brs, 1H), 5.62 (brs, 1H), 5.44 (t, J=13.7 Hz, 1H), 5.20-4.96 (brs, 2H), 4.96-4.76 (m, 2H), 4.58 (s, 2H), 4.15 (s, 1H), 3.86 (t, J=4.8 Hz, 2H), 3.73-3.56 (m, 6H), 3.45 (t, J=4.5 Hz, 4H), 3.17-3.12 (m, 1H), 2.82-2.71 (m, 4H), 2.50 (s, 1H), 2.28-2.00 (m, 3H), 1.95-1.33 (m, 4H); [M+H]+=899.8.

### Example 8: (7S)-7-(1-(1-(2-(2-(2-(2-((2-(2, 6-dioxopiperidin-3-yl)-1, 3-dioxoisoindolin-4-yl)amino)ethoxy)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

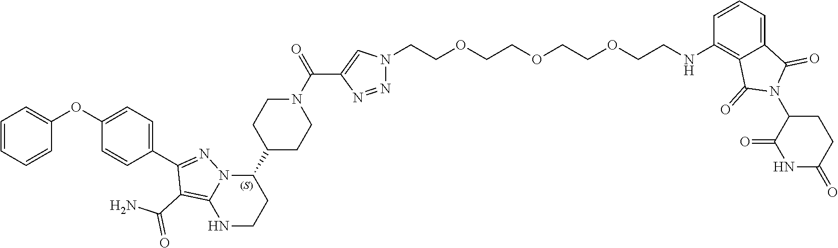

The titled compound was synthesized in the procedures similar to Example 1. ¹H NMR (400 MHz, DMSO) δ_{H} 11.06 (s, 1H), 8.45 (s, 1H), 7.56-7.47 (m, 3H), 7.47-7.37 (m, 2H), 7.22-7.12 (m, 2H), 7.10-7.02 (m, 4H), 6.99 (d, J=4.0 Hz, 1H), 6.88 (dd, J=8.0, 4.0 Hz, 1H), 6.68 (s, 1H), 5.03 (dd, J=12.0, 4.0 Hz, 1H), 4.85 (d, J=12.0 Hz, 1H), 4.58-4.49 (m, 3H), 4.02 (d, J=4.0 Hz, 1H), 3.82 (t, J=4.0 Hz, 2H), 3.57 (t, J=4.0 Hz, 2H), 3.54-3.44 (m, 8H), 3.30-3.24 (m, 1H), 3.15-3.05 (m, 1H), 2.92-2.81 (m, 1H), 2.77-2.64 (m, 1H), 2.61-2.53 (m, 1H), 2.36-2.26 (m, 1H), 2.10-1.85 (m, 3H), 1.71-1.82 (m, 1H), 1.66-1.56 (m, 1H), 1.46-1.24 (m, 2H); [M+H]⁺=943.8.

### Example 9: (7S)-7-(1-(1-(2-(2-(2-((3-(2-(2, 6-dioxopiperidin-3-yl)-1-oxoisoindolin-4-yl)prop-2-yn-1-yl)oxy)ethoxy)ethoxy)ethyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

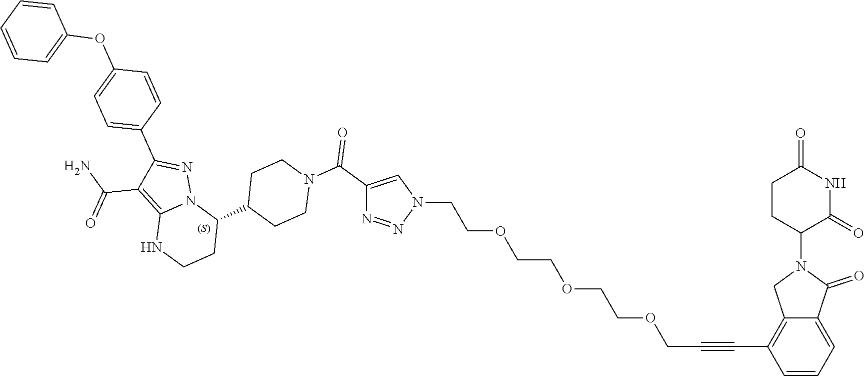

### Example 10: (7S)-7-(1-(1-(15-(2-(2, 6-dioxopiperidin-3-yl)-1-oxoisoindolin-4-yl)-3, 6, 9, 12-tetraoxapentadecyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

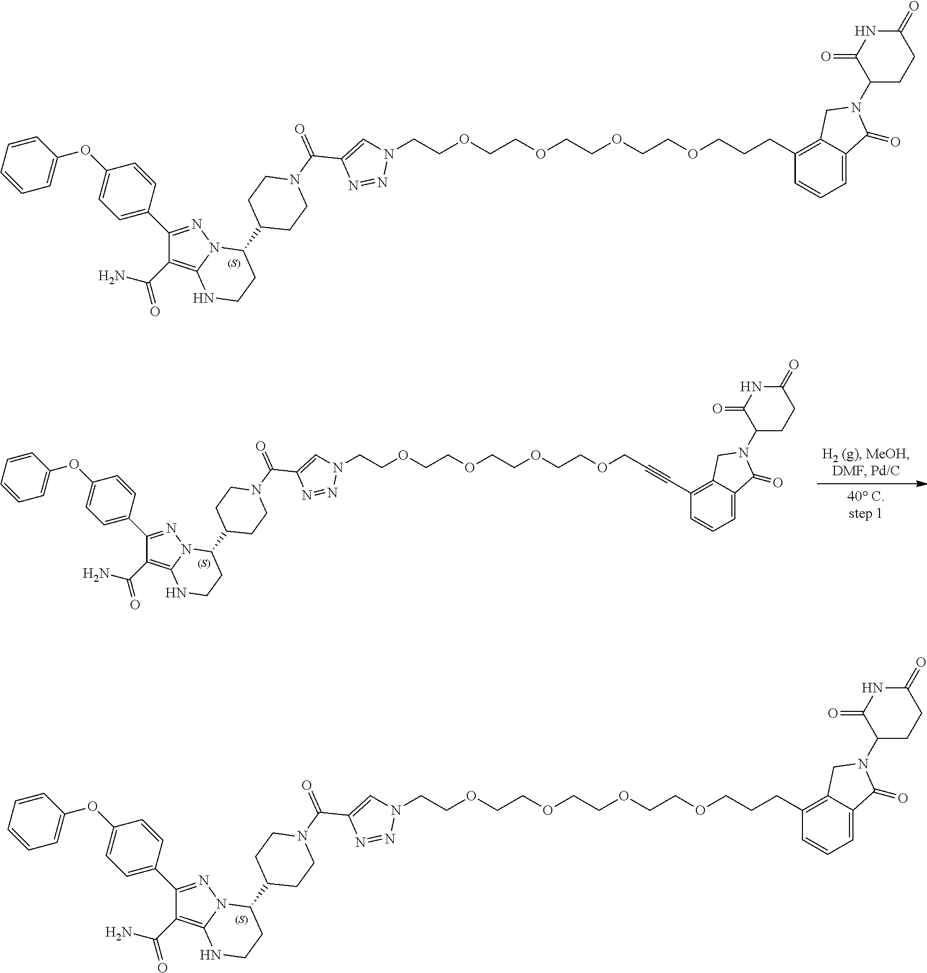

### Step 1: (7S)-7-(1-(1-(15-(2-(2, 6-dioxopiperidin-3-yl)-1-oxoisoindolin-4-yl)-3, 6, 9, 12-tetraoxapentadecyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

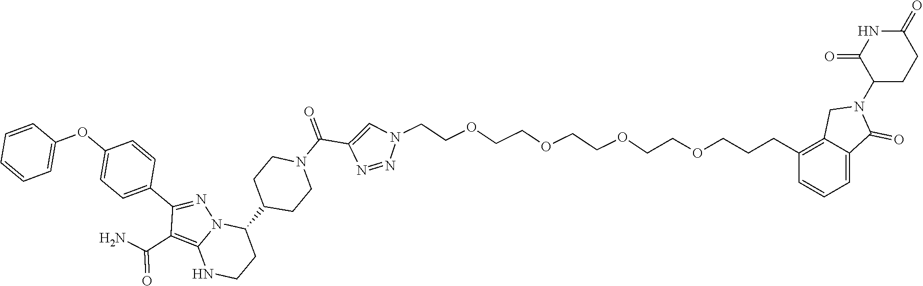 [SMILES: NC(=O)c1c(-c2ccc(Oc3ccccc3)cc2)nn2c1NCC[C@H]2C1CCN(C(=O)c2cn(CCOCCOCCOCCOCCCc3cccc4c3CN(C3CCC(=O)NC3=O)C4=O)nn2)CC1]

A mixture of (7S)-7-(1-(1-(15-(2-(2, 6-dioxopiperidin-3-yl)-1-oxoisoindolin-4-yl)-3, 6, 9, 12-tetraoxapentadec-14-yn-1-yl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide (190 mg, 0.20 mmol, 1 eq) in MeOH (4 mL) and DMF (3 mL) was stirred in a round bottom flask for 2 min at R.T. under the atmosphere of N₂. Pd/C was added. N₂ in bottom was replaced with H₂. The mixture was stirred at 38° C. for 12 h under H₂. The reaction was monitored with TLC. When the reaction was completed, black solid was removed from the solution via filtration. The solvent was evaporated in vacuum to afford the crude product, which was further purified with pre-HPLC to give the product (116.37 mg, 61%). ¹H NMR (400 MHz, CDCl₃) δ_{H} 8.68-8.64 (m, 1H), 8.29 (s, 1H), 7.74 (d, J=7.1 Hz, 1H), 7.46-7.36 (m, 6H), 7.21-6.96 (m, 5H), 6.50 (brs, 1H), 5.61 (brs, 2H), 5.31-5.25 (m, 2H), 4.82-4.78 (m, 1H), 4.66-4.40 (m, 3H), 4.33 (d, J=16.1 Hz, 1H), 4.15 (s, 1H), 3.88-3.87 (m, 2H), 3.73-3.37 (m, 16H), 3.17-3.13 (m, 1H), 2.93-2.71 (m, 5H), 2.58-2.31 (m, 2H), 2.29-2.00 (m, 3H), 1.99-1.77 (m, 3H), 1.76-1.34 (m, 3H); [M+H]⁺=972.8.

### Example 11: (7S)-7-(1-(1-(15-(2-(2, 6-dioxopiperidin-3-yl)-1-oxoisoindolin-4-yl)-3, 6, 9, 12-tetraoxapentadec-14-yn-1-yl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

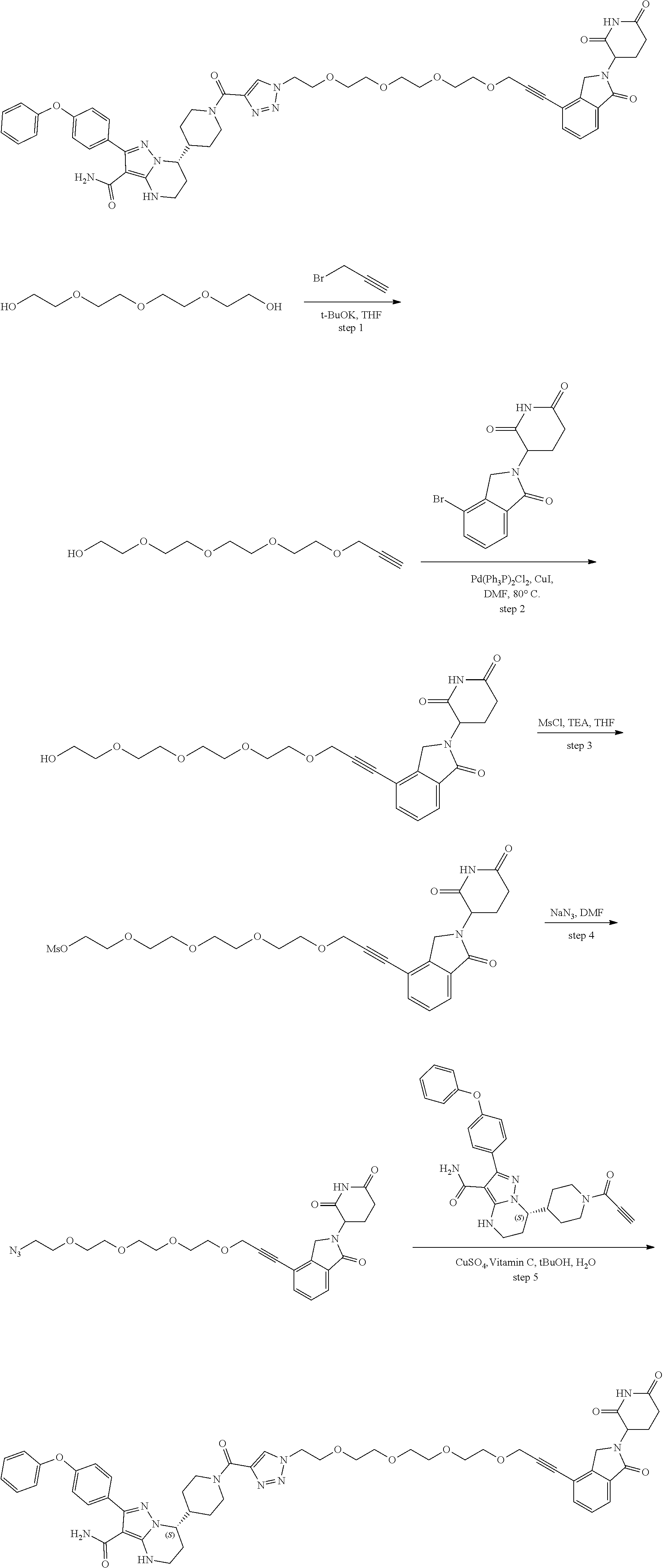

### Step 1: 3, 6, 9, 12-tetraoxapentadec-14-yn-1-ol

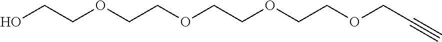 [SMILES: C#CCOCCOCCOCCOCCO]

A mixture of 2, 2′-((oxybis(ethane-2,1-diyl))bis(oxy))bis(ethan-1-01) (15.85 g, 81.7 mmol, 1.8 eq) and t-BuOK (5.08 g, 45.4 mmol, 1 eq) in dry THF (50 mL) was stirred in a round bottom flask at R.T. for 20 min under the atmosphere of N₂. Then, 3-bromoprop-1-yne (90% in Toluene, 6.0 g, 45.4 mmol, 1 eq) was added dropwise. The mixture was stirred at R.T. for 12 h under the atmosphere of N₂. The reaction was monitored with TLC (stained by KMnO₄). When the reaction was completed, solvent was evaporated in vacuum and saturated aqueous NaCl was added to quench the reaction. The mixture was acidified with 1M HCl to pH=3-5 and extracted with DCM. The organic layer was dried over anhydrous Na₂SO₄, and evaporated in vacuum to afford the crude product, which was further purified with silica gel column chromatography (DCM:MeOH=10:1) to give the product (7.95 g, 76%).

### Step 2: 3-(4-(1-hydroxy-3, 6, 9, 12-tetraoxapentadec-14-yn-15-yl)-1-oxoisoindolin-2-yl)piperidine-2, 6-dione

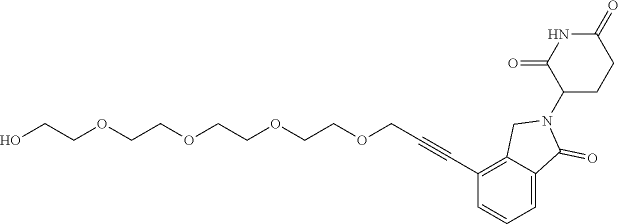 [SMILES: O=C1CCC(N2Cc3c(C#CCOCCOCCOCCOCCO)cccc3C2=O)C(=O)N1]

A mixture of 3-(4-bromo-1-oxoisoindolin-2-yl)piperidine-2, 6-dione (427 mg, 1.32 mmol, 1 eq), 3, 6, 9, 12-tetraoxapentadec-14-yn-1-ol (613.4 mg, 2.64 mmol, 2 eq), bis(triphenylphosphine) palladium(II) chloride (93 mg, 0.13 mmol, 0.1 eq) and CuI (50 mg, 0.26 mmol, 0.2 eq) in dry DMF (6.6 mL) was stirred in a round bottom flask at R.T. for 2 min under the atmosphere of N₂. Then, dry Et₃N (3.3 mL) was added. The mixture was stirred at 80° C. for 3 h under the atmosphere of N₂. The reaction was monitored with TLC. When the reaction was completed, the solvent was evaporated in vacuum. The crude product thus obtained was further purified with silica gel column chromatography (DCM:MeOH=40:1-10:1 gradient elution) to give the product (292 mg, 46%). [M+H]⁺=474.9.

### Step 3: 15-(2-(2, 6-dioxopiperidin-3-yl)-1-oxoisoindolin-4-yl)-3, 6, 9, 12-tetraoxapentadec-14-yn-1-yl methanesulfonate

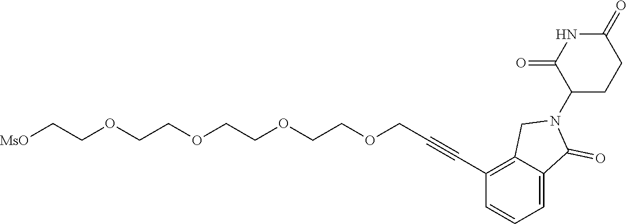 [SMILES: CS(=O)(=O)OCCOCCOCCOCCOCC#Cc1cccc2c1CN(C1CCC(=O)NC1=O)C2=O]

A mixture of 3-(4-(1-hydroxy-3, 6, 9, 12-tetraoxapentadec-14-yn-15-yl)-1-oxoisoindolin-2-yl)piperidine-2, 6-dione (292 mg, 0.61 mmol, 1 eq) and Et₃N (0.17 mL, 1.23 mmol, 2 eq) in dry THF (1.8 mL) was stirred in a round bottom flask at 0° C. for 5 min under the atmosphere of N₂. Then, MSCl (97.1 μL, 1.24 mmol, 2 eq) was added. The mixture was warmed to R.T. and stirred for 4 h under the atmosphere of N₂. The reaction was monitored with TLC. When the reaction was completed, the solvent was evaporated in vacuum. The crude product thus obtained was further purified with silica gel column chromatography (DCM:MeOH=40:1˜10:1 gradient elution) to give the product (305 mg, 90%). [M+H]⁺=552.9.

### Step 4: 3-(4-(1-azido-3, 6, 9, 12-tetraoxapentadec-14-yn-15-yl)-1-oxoisoindolin-2-yl)piperidine-2, 6-dione

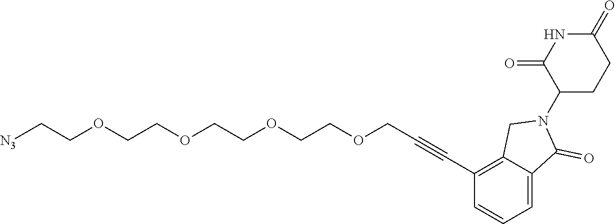 [SMILES: [N-]=[N+]=NCCOCCOCCOCCOCC#Cc1cccc2c1CN(C1CCC(=O)NC1=O)C2=O]

A mixture of 15-(2-(2, 6-dioxopiperidin-3-yl)-1-oxoisoindolin-4-yl)-3, 6, 9, 12-tetraoxapentadec-14-yn-1-yl methanesulfonate (305 mg, 0.55 mmol) and NaN₃ (46.7 mg, 0.72 mmol) in dry DMF (4 mL) was stirred in a round bottom flask at 80° C. for 4 h under the atmosphere of N₂. The reaction was monitored with TLC. When the reaction was completed, saturated aqueous NaCl was added to quench the reaction and extracted with EtOAc. The organic layer was dried over anhydrous Na₂SO₄ and evaporated in vacuum to afford the crude product, which was further purified with silica gel column chromatography (DCM:MeOH=20:1) to give the product (214 mg, 78%). [M+H]⁺=499.9.

### Step 5: (7S)-7-(1-(1-(15-(2-(2, 6-dioxopiperidin-3-yl)-1-oxoisoindolin-4-yl)-3, 6, 9, 12-tetraoxapentadec-14-yn-1-yl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

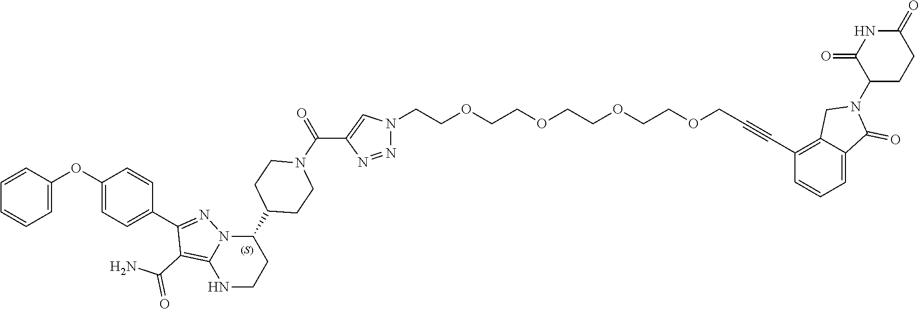

A mixture of 3-(4-(1-azido-3, 6, 9, 12-tetraoxapentadec-14-yn-15-yl)-1-oxoisoindolin-2-yl)piperidine-2, 6-dione (214 mg, 0.43 mmol), (S)-2-(4-phenoxyphenyl)-7-(1-propioloylpiperidin-4-yl)-4, 5, 6, 7-tetrahydropyrazolo[1,5-a]pyrimidine-3-carboxamide (201.2 mg, 0.43 mmol), CuSO₄.5H₂O (107 mg, 0.43 mmol), ascorbate acid (226 mg, 1.28 mmol) and H₂O (0.7 mL) in t-BuOH (6 mL) was stirred in a round bottom flask for 7 h at 70° C. under the atmosphere of N₂. The reaction was quenched with saturated aqueous NaHCO₃ and extracted with DCM. The organic layer was dried over anhydrous Na₂SO₄, and evaporated in vacuum to afford the crude product, which was further purified with silica gel column chromatography (DCM:MeOH=10:1) to give the product (326 mg, 79%). ¹H NMR (400 MHz, CDCl₃) δ_{H} 8.75-8.52 (m, 1H), 8.28 (s, 1H), 7.85 (d, J=7.7 Hz, 1H), 7.62 (d, J=7.6 Hz, 1H), 7.56-7.33 (m, 5H), 7.18-7.05 (m, 5H), 6.86 (brs, 1H), 6.49 (brs, 1H), 5.58 (brs, 1H), 5.31-5.24 (m, 2H), 4.81 (s, 1H), 4.68-4.28 (m, 8H), 4.14 (s, 1H), 3.88 (s, 2H), 3.81-3.54 (m, 11H), 3.45 (s, 2H), 3.15 (s, 1H), 2.94-2.69 (m, 3H), 2.51-2.30 (m, 2H), 2.23-2.14 (m, 2H), 1.84-1.55 (m, 4H); [M+H]⁺=968.8.

### Example 12: (7S)-7-(1-(1-(14-((2-(2, 6-dioxopiperidin-3-yl)-1, 3-dioxoisoindolin-5-yl)amino)-3, 6, 9, 12-tetraoxatetradecyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

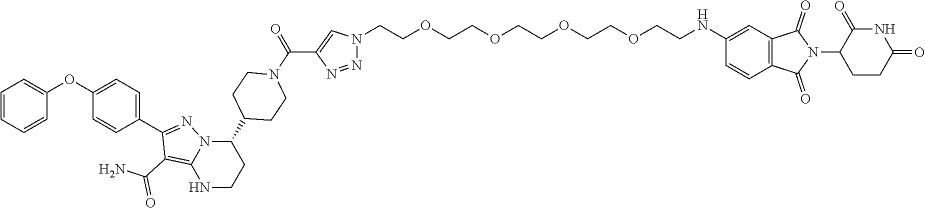 [SMILES: NC(=O)c1c(-c2ccc(Oc3ccccc3)cc2)nn2c1NCC[C@H]2C1CCN(C(=O)c2cn(CCOCCOCCOCCOCCNc3ccc4c(c3)C(=O)N(C3CCC(=O)NC3=O)C4=O)nn2)CC1]

The titled compound was synthesized in the procedures similar to Example 1. ¹H NMR (400 MHz, DMSO) δ_{H} 11.12 (s, 1H), 8.50 (s, 1H), 7.53-7.64 (m, 3H), 7.47 (t, J=8.0 Hz, 2H), 7.26-7.19 (m, 2H), 7.17-7.09 (m, 4H), 7.06 (d, J=1.6 Hz, 1H), 6.94 (dd, J=8.0, 2.0 Hz, 1H), 6.74 (s, 1H), 5.09 (dd, J=12.0, 4.0 Hz, 1H), 4.91 (d, J=12.0 Hz, 1H), 4.66-4.56 (m, 3H), 4.09 (dd, J=10.8, 5.6 Hz, 1H), 3.89 (t, J=4.8 Hz, 2H), 3.63 (t, J=5.4 Hz, 2H), 3.61-3.55 (m, 6H), 3.55-3.47 (m, 6H), 3.44-3.40 (m, 1H), 3.38-3.34 (m, 2H), 2.99-2.85 (m, 1H), 2.84-2.71 (m, 1H), 2.67-2.57 (m, 3H), 2.39 (s, 1H), 2.15-1.93 (m, 3H), 1.83 (t, J=8.0 Hz, 1H), 1.68 (t, J=12.0 Hz, 1H), 1.48-1.31 (m, 2H); [M+H]⁺=988.0.

### Example 13: (7S)-7-(1-(1-(14-((2-(2, 6-dioxopiperidin-3-yl)-1, 3-dioxoisoindolin-4-yl)amino)-3, 6, 9, 12-tetraoxatetradecyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

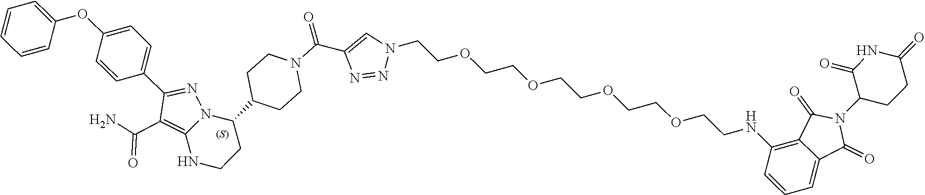

### Example 14: (7S)-7-(1-(1-(17-((2-(2, 6-dioxopiperidin-3-yl)-1, 3-dioxoisoindolin-5-yl)amino)-3, 6, 9, 12, 15-pentaoxaheptadecyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

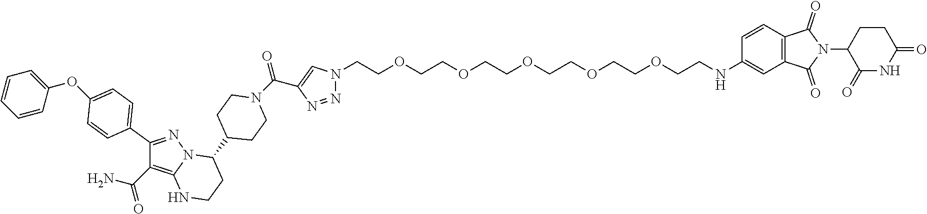 [SMILES: NC(=O)c1c(-c2ccc(Oc3ccccc3)cc2)nn2c1NCC[C@H]2C1CCN(C(=O)c2cn(CCOCCOCCOCCOCCOCCNc3ccc4c(c3)C(=O)N(C3CCC(=O)NC3=O)C4=O)nn2)CC1]

The titled compound was synthesized in the procedures similar to Example 1. ¹H NMR (400 MHz, DMSO) δ_{H} 11.06 (s, 1H), 8.45 (s, 1H), 7.55 (d, J=8.0 Hz, 1H), 7.50 (d, J=8.0 Hz, 2H), 7.41 (t, J=8.0 Hz, 2H), 7.20-7.12 (m, 2H), 7.12-6.98 (m, 5H), 6.89 (dd, J=8.0, 2.0 Hz, 1H), 6.68 (s, 1H), 5.03 (dd, J=12.0, 4.0 Hz, 1H), 4.85 (d, J=12.0 Hz, 1H), 4.59-4.49 (m, 3H), 4.03 (dd, J=12.0, 6.0 Hz, 1H), 3.83 (t, J=4.0 Hz, 2H), 3.58 (t, J=4.0 Hz, 3H), 3.55-3.42 (m, 17H), 3.37-3.36 (m, 1H), 3.31-3.28 (m, 1H), 3.10 (dd, J=32.0, 12.0 Hz, 1H), 2.93-2.81 (m, 1H), 2.75-2.65 (m, 1H), 2.61-2.54 (m, 1H), 2.33 (s, 1H), 2.07-1.87 (m, 3H), 1.77 (t, J=12.0 Hz, 1H), 1.62 (t, J=12.0 Hz, 1H), 1.43-1.26 (m, 1H); [M+2H]²⁺=516.6.

### Example 15: (7S)-7-(1-(1-(17-((2-(2, 6-dioxopiperidin-3-yl)-1, 3-dioxoisoindolin-4-yl)amino)-3, 6, 9, 12, 15-pentaoxaheptadecyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

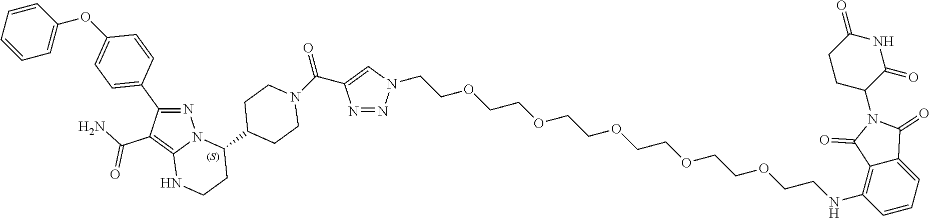

The titled compound was synthesized in the procedures similar to Example 1. ¹H NMR (400 MHz, DMSO) δ_{H} 11.09 (s, 1H), 8.44 (s, 1H), 7.49-7.59 (m, 3H), 7.39-7.43 (m, 2H), 7.02-7.19 (m, 7H), 6.68 (s, 1H), 6.60 (t, J=4.0 Hz, 1H), 5.03-5.06 (m, 1H), 4.83-4.85 (m, 1H), 4.51-4.57 (m, 3H), 4.00-4.05 (m, 1H), 3.83 (t, J=4.0 Hz, 2H), 3.60 (t, J=4.0 Hz, 2H), 3.45-3.55 (m, 18H), 3.28-3.30 (m, 2H), 3.04-3.15 (m, 1H), 2.83-2.92 (m, 1H), 2.65-2.76 (m, 1H), 2.52-2.60 (m, 3H), 2.26-2.38 (m, 1H), 1.89-2.06 (m, 3H), 1.71-1.80 (m, 1H), 1.58-1.66 (m, 1H), 1.26-1.45 (m, 2H); [M+2H]²⁺=516.5.

### Example 16: (7S)-7-(1-(1-(20-((2-(2, 6-dioxopiperidin-3-yl)-1, 3-dioxoisoindolin-4-yl)amino)-3, 6, 9, 12, 15, 18-hexaoxaicosyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

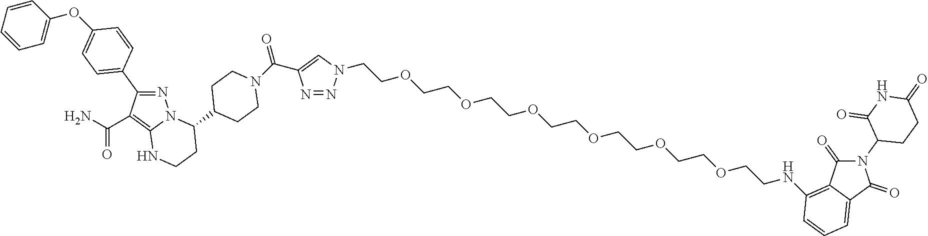 [SMILES: NC(=O)c1c(-c2ccc(Oc3ccccc3)cc2)nn2c1NCC[C@H]2C1CCN(C(=O)c2cn(CCOCCOCCOCCOCCOCCOCCNc3cccc4c3C(=O)N(C3CCC(=O)NC3=O)C4=O)nn2)CC1]

The titled compound was synthesized in the procedures similar to Example 1. ¹H NMR (400 MHz, CDCl₃) δ_{H} 8.62 (brs, 1H), 8.30 (s, 1H), 7.51-7.47 (m, 3H), 7.38 (t, J=7.9 Hz, 2H), 7.16 (t, J=7.4 Hz, 1H), 7.11-7.05 (m, 5H), 6.91 (d, J=8.4 Hz, 1H), 6.52 (brs, 1H), 5.58 (brs, 1H), 5.31 (s, 1H), 5.00-4.70 (m, 2H), 4.57 (s, 2H), 4.39-4.00 (m, 3H), 3.88 (s, 2H), 3.80-3.31 (m, 25H), 3.14 (s, 1H), 2.86-2.72 (m, 4H), 2.48 (s, 1H), 2.13-2.10 (m, 3H), 1.84-1.69 (m, 5H); [M+2H]²⁺=538.5.

### Example 17: (7S)-7-(1-(1-(23-((2-(2, 6-dioxopiperidin-3-yl)-1, 3-dioxoisoindolin-4-yl)amino)-3, 6, 9, 12, 15, 18, 21-heptaoxatricosyl)-1H-1, 2, 3-triazole-4-carbonyl)piperidin-4-yl)-2-(4-phenoxyphenyl)-4, 5, 6, 7-tetrahydropyrazolo[1, 5-a]pyrimidine-3-carboxamide

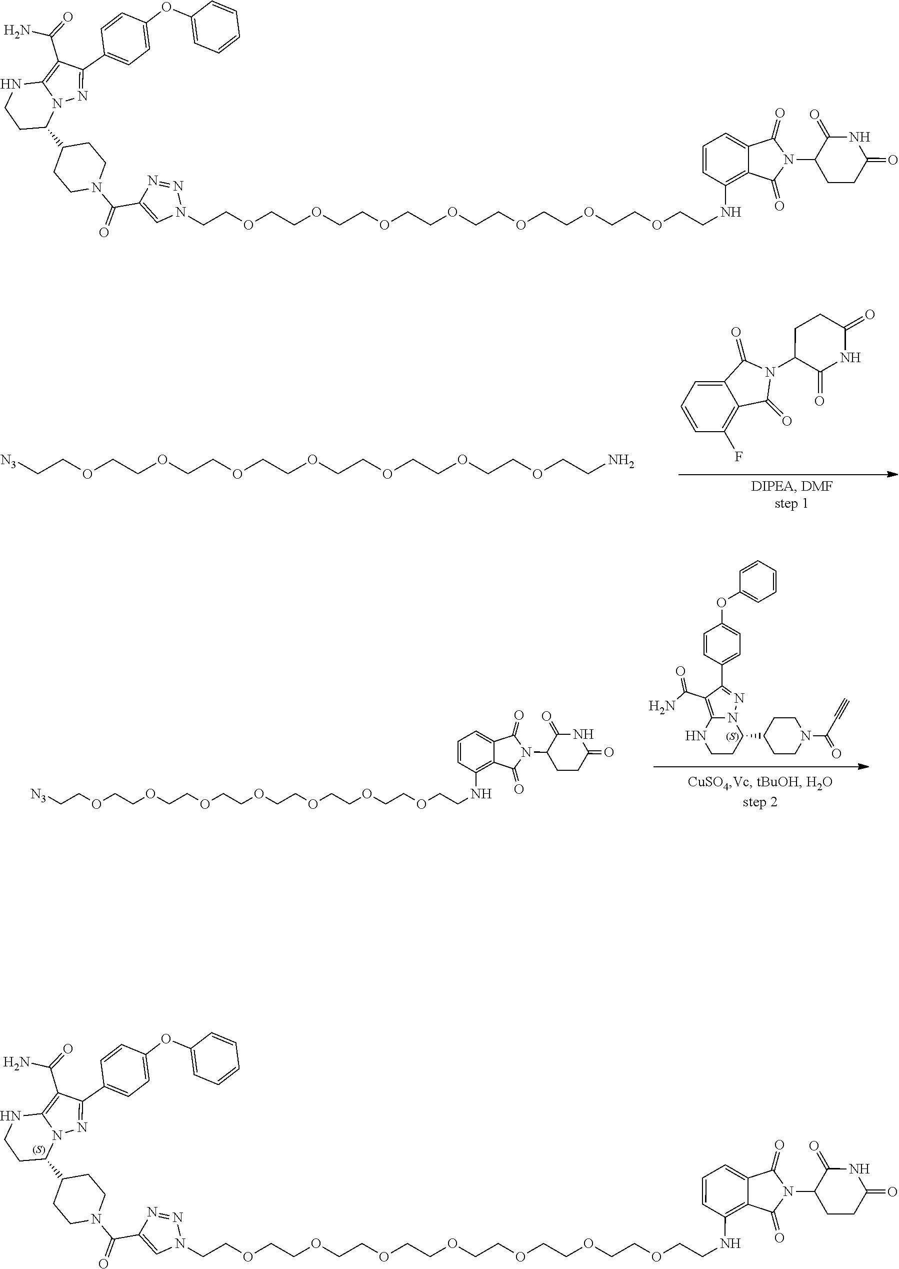

### Step 1: 4-((23-azido-3, 6, 9, 12, 15, 18, 21-heptaoxatricosyl)amino)-2-(2, 6-dioxopiperidin-3-yl)isoindoline-1, 3-dione

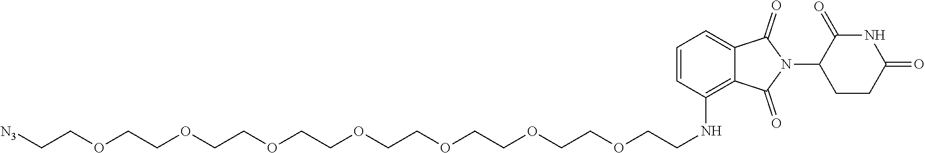 [SMILES: [N-]=[N+]=NCCOCCOCCOCCOCCOCCOCCOCCNc1cccc2c1C(=O)N(C1CCC(=O)NC1=O)C2=O]

A mixture of 23-azido-3, 6, 9, 12, 15, 18, 21-heptaoxatricosan-1-amine (870.2 mg, 2.21 mmol), 2-(2, 6-dioxopiperidin-3-yl)-4-fluoroisoindoline-1, 3-dione (508 mg, 1.84 mmol), DIEA (1.02 mL) in dry DMF (4.4 mL) was stirred in a round bottom flask for 3 h at 90° C. The reaction was quenched with water and the mixture was washed once with saturated aqueous NaCl, then extracted with EtOAc. The organic layer was dried over anhydrous Na₂SO₄, and evaporated in vacuum to afford the crude product, which was further purified with silica gel column chromatography (DCM:MeOH=20:1˜4:1 gradient elution) to give the product (331 mg, 28%). [M+H]⁺=650.9.

### Step 2: (7S)-7-(1-(1-(23-((2-(2, 6-dioxopiperidin-3-yl)-1, 3-dioxoisoindolin-4-yl)amino)-3, 6, 9, 12, 15, 18, 21-heptaoxatricosyl)-1H-1, 2, 3-triazole-4-carbonyl)piperidin-4-yl)-2-(4-phenoxyphenyl)-4, 5, 6, 7-tetrahydropyrazolo[1,5-a]pyrimidine-3-carboxamide

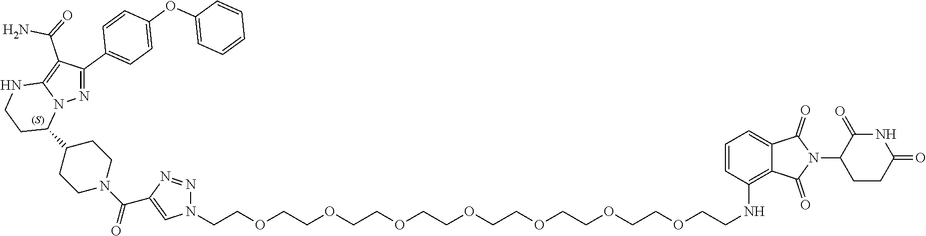

A mixture of (S)-2-(4-phenoxyphenyl)-7-(1-propioloylpiperidin-4-yl)-4, 5, 6, 7-tetrahydropyrazolo[1,5-a]pyrimidine-3-carboxamide (81 mg, 0.17 mmol), 4-((23-azido-3, 6, 9, 12, 15, 18, 21-heptaoxatricosyl)amino)-2-(2, 6-dioxopiperidin-3-yl)isoindoline-1, 3-dione (112 mg, 0.17 mmol, 1 eq), CuSO₄.5H₂O (43 mg, 0.17 mmol), ascorbic acid (90.8 mg, 0.52 mmol) and H₂O (1.38 mL) in t-BuOH (13.8 mL) was stirred in a round bottom flask for 5 hat 70° C. under the atmosphere of N₂. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (43.53 mg, 23%). ¹H NMR (400 MHz, CDCl₃) δ_{H} 8.59 (brs, 1H), 8.26 (s, 1H), 7.57-7.44 (m, 3H), 7.38 (t, J=8.0 Hz, 2H), 7.16 (t, J=7.4 Hz, 1H), 7.14-7.05 (m, 5H), 6.92 (d, J=8.5 Hz, 1H), 6.51 (brs, 2H), 5.58 (brs, 1H), 5.34-5.30 (m, 1H), 5.00-4.72 (m, 2H), 4.57 (t, J=4.6 Hz, 2H), 4.16-4.12 (m, 1H), 3.88 (t, J=4.6 Hz, 2H), 3.67-3.61 (m, 25H), 3.47-3.46 (m, 4H), 3.17-3.13 (m, 1H), 2.96-2.65 (m, 4H), 2.51-2.47 (m, 1H), 2.13-2.10 (m, 3H), 1.99-1.12 (m, 5H); [M+2H]²⁺=560.5.

### Example 18: (7S)-7-(1-(4-(1-(2-(2-((2-(2, 6-dioxopiperidin-3-yl)-1, 3-dioxoisoindolin-4-yl)amino)ethoxy)ethyl)-1H-1, 2, 3-triazol-4-yl)butanoyl)piperidin-4-yl)-2-(4-phenoxyphenyl)-4, 5, 6, 7-tetrahydropyrazolo[1, 5-a]pyrimidine-3-carboxamide

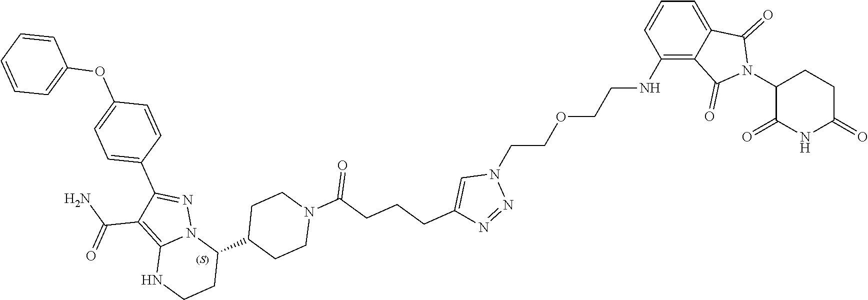

The titled compound was synthesized in the procedures similar to Example 21. ¹H NMR (400 MHz, DMSO) δ_{H} 10.84 (s, 1H), 7.71 (s, 1H), 7.58-7.47 (m, 3H), 7.40 (t, J=7.6 Hz, 2H), 7.15 (t, J=7.2 Hz, 1H), 7.10-6.99 (m, 6H), 6.54 (brs, 2H), 5.88 (s, 2H), 5.01 (dd, J=12.6, 5.2 Hz, 1H), 4.46 (t, J=4.8 Hz, 2H), 4.00 (d, J=5.1 Hz, 1H), 3.83 (s, 2H), 3.61 (s, 2H), 3.43 (d, J=5.1 Hz, 2H), 3.31 (s, 2H), 2.94-2.81 (m, 2H), 2.87-2.51 (m, 5H), 2.35-2.14 (m, 4H), 1.99 (dd, J=34.7, 8.7 Hz, 3H), 1.85-1.75 (m, 2H), 1.70 (d, J=4 Hz, 1H), 1.53 (d, J=4 Hz, 1H), 1.25 (s, 3H); [M+H]⁺=897.9.

### Example 19: (7S)-7-(1-(4-(1-(4-(2-((2-(2, 6-dioxopiperidin-3-yl)-1, 3-dioxoisoindolin-4-yl)oxy)acetamido)butyl)-1H-1, 2, 3-triazol-4-yl)butanoyl)piperidin-4-yl)-2-(4-phenoxyphenyl)-4, 5, 6, 7-tetrahydropyrazolo[1,5-a]pyrimidine-3-carboxamide

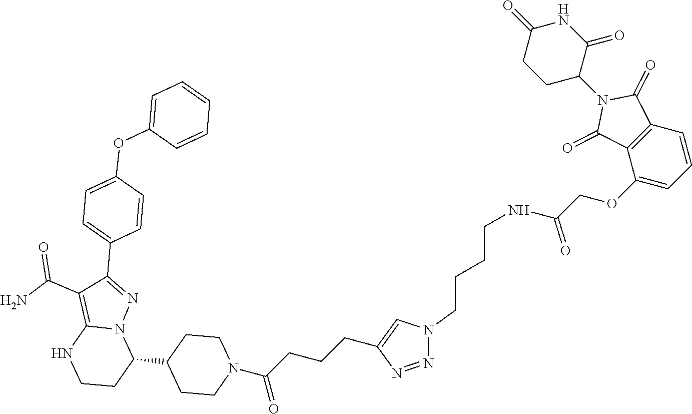 [SMILES: NC(=O)c1c(-c2ccc(Oc3ccccc3)cc2)nn2c1NCC[C@H]2C1CCN(C(=O)CCCc2cn(CCCCNC(=O)COc3cccc4c3C(=O)N(C3CCC(=O)NC3=O)C4=O)nn2)CC1]

### Example 20: (7S)-7-(1-(4-(1-(2-(2-(2-((2-(2, 6-dioxopiperidin-3-yl)-1, 3-dioxoisoindolin-4-yl)amino)ethoxy)ethoxy)ethyl)-1H-1, 2, 3-triazol-4-yl)butanoyl)piperidin-4-yl)-2-(4-phenoxyphenyl)-4, 5, 6, 7-tetrahydropyrazolo[1,5-a]pyrimidine-3-carboxamide

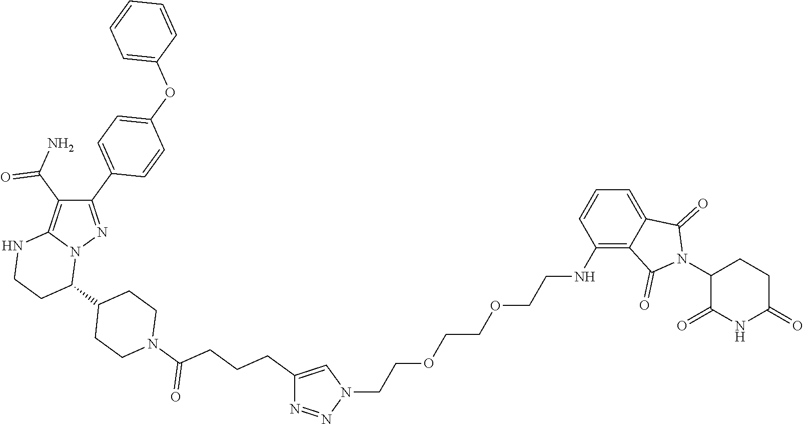 [SMILES: NC(=O)c1c(-c2ccc(Oc3ccccc3)cc2)nn2c1NCC[C@H]2C1CCN(C(=O)CCCc2cn(CCOCCOCCNc3cccc4c3C(=O)N(C3CCC(=O)NC3=O)C4=O)nn2)CC1]

The titled compound was synthesized in the procedure similar to Example 21. ¹H NMR (400 MHz, DMSO) δ_{H} 10.82 (s, 1H), 7.71 (s, 1H), 7.55 (t, J=7.8 Hz, 1H), 7.50 (d, J=8.2 Hz, 2H), 7.40 (t, J=7.6 Hz, 2H), 7.19-6.99 (m, 7H), 6.54 (d, J=19.0 Hz, 2H), 5.87 (s, 2H), 5.00 (dd, J=12.6, 5.4 Hz, 1H), 4.41 (dd, J=15.0, 9.9 Hz, 3H), 4.00 (d, J=5.4 Hz, 1H), 3.80 (t, J=5.1 Hz, 2H), 3.60 (t, J=5.1 Hz, 2H), 3.53 (s, 4H), 3.43 (d, J=5.3 Hz, 2H), 3.32 (s, 2H), 2.93-2.79 (m, 2H), 2.66-2.52 (m, 5H), 2.31-2.00 (m, 4H), 1.99-1.90 (m, 3H), 1.86-1.76 (m, 2H), 1.70 (d, J=11.8 Hz, 1H), 1.54 (d, J=12.2 Hz, 1H), 1.25 (s, 3H); [M+H]⁺=941.8.

### Example 21: (7S)-7-(1-(4-(1-(2-(2-(2-(2-((2-(2, 6-dioxopiperidin-3-yl)-1, 3-dioxoisoindolin-4-yl)amino)ethoxy)ethoxy)ethoxy)ethyl)-1H-1, 2, 3-triazol-4-yl)butanoyl)piperidin-4-yl)-2-(4-phenoxyphenyl)-4, 5, 6, 7-tetrahydropyrazolo[1, 5-a]pyrimidine-3-carboxamide

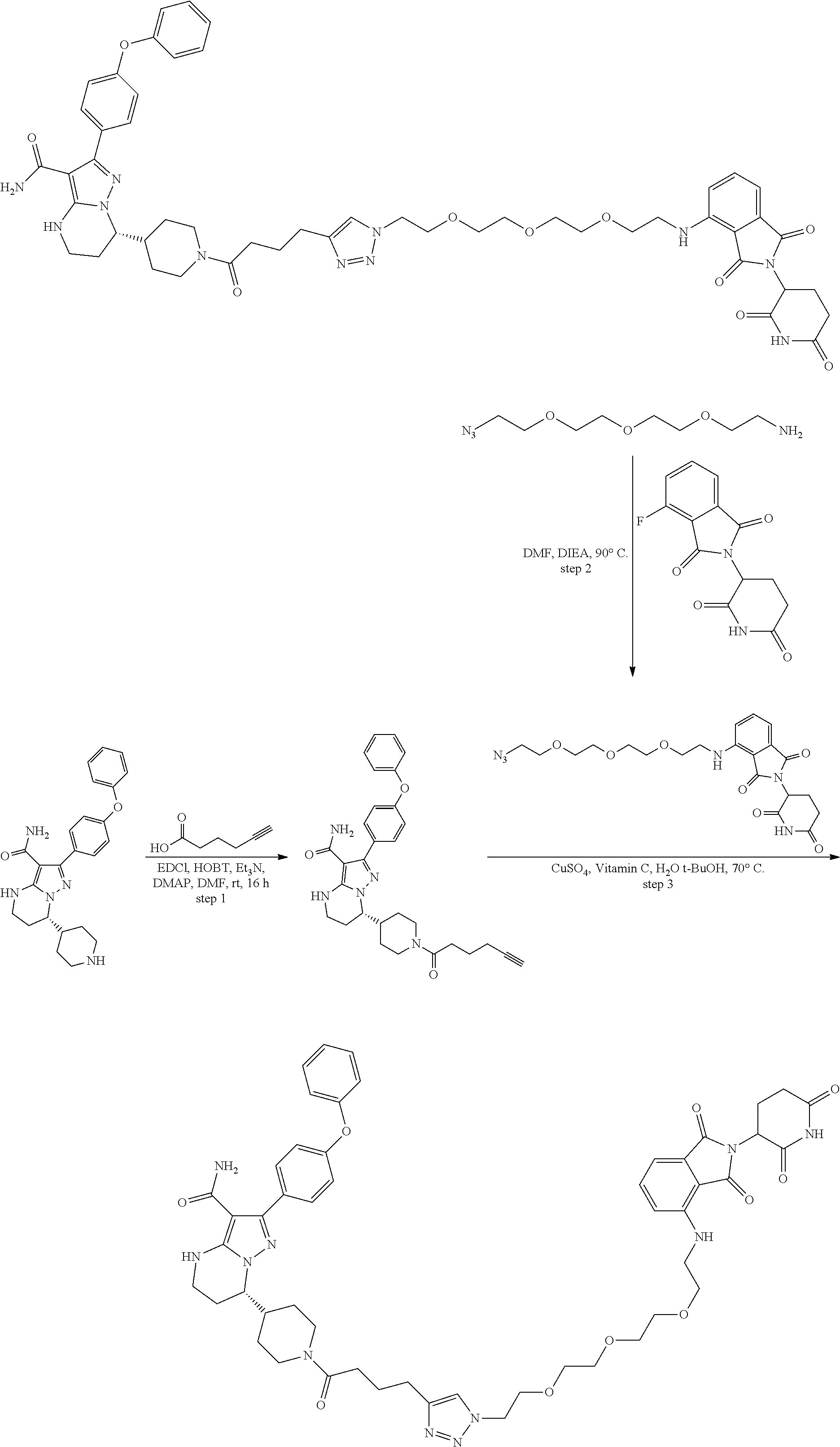

### Step 1: (S)-7-(1-(hex-5-ynoyl)piperidin-4-yl)-2-(4-phenoxyphenyl)-4, 5, 6, 7-tetrahydro-pyrazolo[1, 5-a]pyrimidine-3-carboxamide

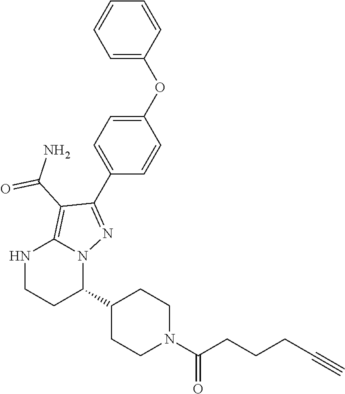 [SMILES: C#CCCCC(=O)N1CCC([C@@H]2CCNc3c(C(N)=O)c(-c4ccc(Oc5ccccc5)cc4)nn32)CC1]

To a solution of the compound (S)-2-(4-phenoxyphenyl)-7-(piperidin-4-yl)-4, 5, 6, 7-tetrahydropyrazolo[1,5-a]pyrimidine-3-carboxamide (836 mg, 2 mmol), hex-5-ynoic acid (224 mg, 2 mmol) dissolved in DCM (15 mL) were added EDCI (460.1 mg, 2.4 mmol), HOBT (324.3 mg, 2.4 mmol, 1.2 eq) and DMAP (24.4 mg, 0.2 mmol, 0.1 eq). The mixture was stirred at room temperature overnight. The solution was washed with water 30 mL and extracted with DCM (20 mL*3). The organic layers were combined and washed with brine (40 mL*3). The mixture was dried over Na₂SO₄, filtered and concentrated to dryness. The residue was purified with silica gel chromatography (DCM:MeOH=10:1) to afford the product (1 g, 98%). [M+H]⁺=511.9.

### Step 2: 4-((2-(2-(2-(2-azidoethoxy)ethoxy)ethoxy)ethyl)amino)-2-(2, 6-dioxopiperidin-3-yl)isoindoline-1, 3-dione

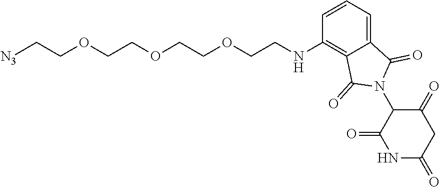 [SMILES: [N-]=[N+]=NCCOCCOCCOCCNc1cccc2c1C(=O)N(C1C(=O)CC(=O)NC1=O)C2=O]

A mixture of 2-(2-(2-(2-azidoethoxy)ethoxy)ethoxy)ethan-1-amine (478 mg, 2.19 mmol), 2-(2, 6-dioxopiperidin-3-yl)-4-fluoroisoindoline-1, 3-dione (605 mg, 2.19 mmol) and DIEA (1.21 mL) in dry DMF (5.2 mL) was stirred in a round bottom flask for 3 h at 90° C. The reaction was quenched with water and the mixture was washed once with saturated aqueous NaCl, then extracted with EtOAc. The organic layer was dried over anhydrous Na₂SO₄, and evaporated in vacuum to afford the crude product, which was further purified with silica gel column chromatography (PE:EA=1:2) to give the product (200 mg, 19%). [M+H]⁺=474.9.

### Step 3: (7S)-7-(1-(4-(1-(2-(2-(2-(2-((2-(2, 6-dioxopiperidin-3-yl)-1, 3-dioxoisoindolin-4-yl)amino)ethoxy)ethoxy)ethoxy)ethyl)-1H-1, 2, 3-triazol-4-yl)butanoyl)piperidin-4-yl)-2-(4-phenoxyphenyl)-4, 5, 6, 7-tetrahydropyrazolo[1,5-a]pyrimidine-3-carboxamide

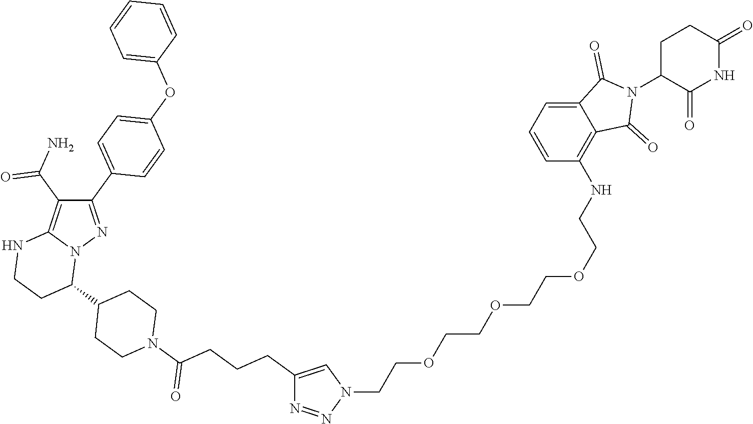 [SMILES: NC(=O)c1c(-c2ccc(Oc3ccccc3)cc2)nn2c1NCC[C@H]2C1CCN(C(=O)CCCc2cn(CCOCCOCCOCCNc3cccc4c3C(=O)N(C3CCC(=O)NC3=O)C4=O)nn2)CC1]

A mixture of (S)-7-(1-(hex-5-ynoyl)piperidin-4-yl)-2-(4-phenoxyphenyl)-4, 5, 6, 7-tetrahydro-pyrazolo[1,5-a]pyrimidine-3-carboxamide (81 mg, 0.16 mmol), 4-((2-(2-(2-(2-azidoethoxy)ethoxy)ethoxy)ethyl)amino)-2-(2, 6-dioxopiperidin-3-yl)isoindoline-1, 3-dione (75 mg, 0.16 mmol), CuSO₄.5H₂O (40 mg, 0.16 mmol), ascorbic acid (84 mg, 0.48 mmol) and H₂O (1.3 mL) in t-BuOH (13 mL) was stirred in a round bottom flask for 5 h at 70° C. under the atmosphere of N₂. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (64 mg, 41%). ¹H NMR (400 MHz, CDCl₃) δ_{H} 8.66 (brs, 1H), 7.66-7.33 (m, 6H), 7.21-6.99 (m, 6H), 6.91 (d, J=8.5 Hz, 1H), 6.51 (brs, 2H), 5.41 (brs, 2H), 4.90 (dd, J=11.5, 5.1 Hz, 1H), 4.77-4.64 (m, 1H), 4.49 (s, 2H), 4.09 (s, 1H), 4.01-3.33 (m, 17H), 3.13-2.64 (m, 6H), 2.49-1.98 (m, 9H), 1.88-1.54 (m, 2H), 1.52-1.17 (m, 2H); [M+H]⁺=985.9.

### Example 22: (7S)-7-(1-(2-(2-((2-(2, 6-dioxopiperidin-3-yl)-1, 3-dioxoisoindolin-5-yl)amino)-2-oxoethoxy)ethyl)piperidin-4-yl)-2-(4-phenoxyphenyl)-4, 5, 6, 7-tetrahydropyrazolo[1,5-a]pyrimidine-3-carboxamide

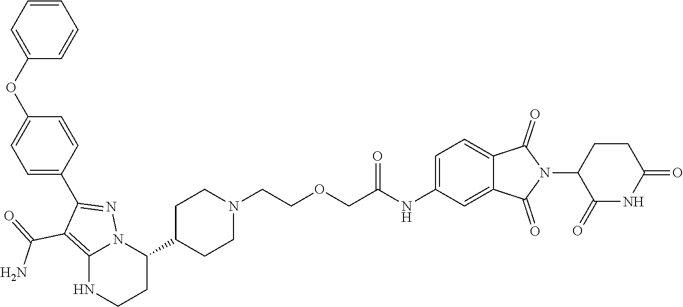 [SMILES: NC(=O)c1c(-c2ccc(Oc3ccccc3)cc2)nn2c1NCC[C@H]2C1CCN(CCOCC(=O)Nc2ccc3c(c2)C(=O)N(C2CCC(=O)NC2=O)C3=O)CC1]

The titled compound was synthesized in the procedures similar to Example 23. ¹H NMR (400 MHz, CDCl₃) δ_{H} 10.27 (brs, 1H), 8.61-8.03 (m, 3H), 7.82 (d, J=8.2 Hz, 1H), 7.56-7.32 (m, 4H), 7.17-7.00 (m, 5H), 6.61 (d, J=8.2 Hz, 1H), 5.15 (brs, 2H), 5.02-4.87 (m, 1H), 4.23-4.16 (m, 3H), 3.99-3.31 (m, 8H), 3.26-2.36 (m, 8H), 2.15-1.92 (m, 5H); [M+H]⁺=774.8.

### Example 23: (7S)-7-(1-(2-(2-(2-(2-((2-(2, 6-dioxopiperidin-3-yl)-1, 3-dioxoisoindolin-5-yl)amino)-2-oxoethoxy)ethoxy)ethoxy)ethyl)piperidin-4-yl)-2-(4-phenoxyphenyl)-4, 5, 6, 7-tetrahydropyrazolo[1,5-a]pyrimidine-3-carboxamide

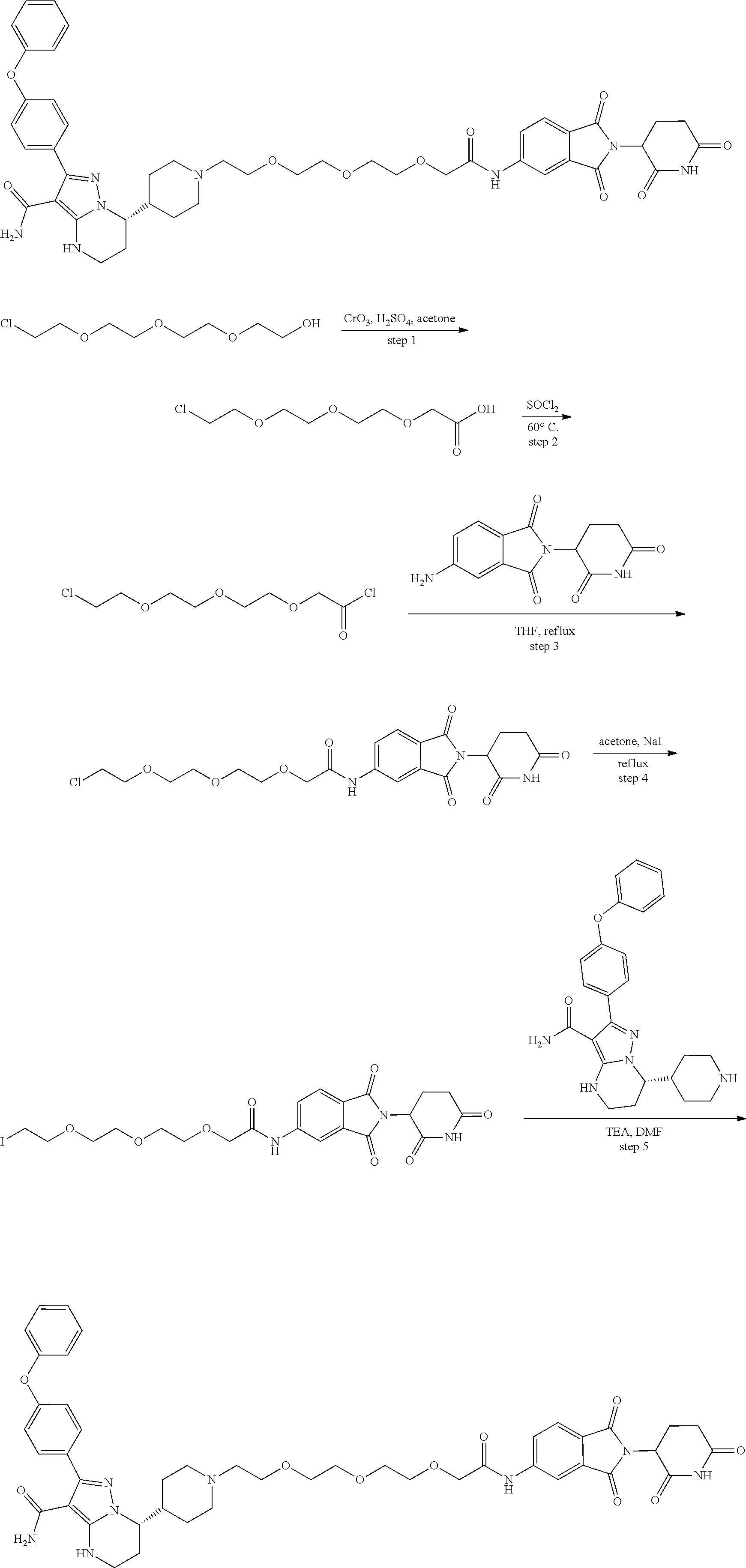

### Step 1: 2-(2-(2-(2-chloroethoxy)ethoxy)ethoxy)acetic Acid

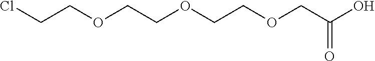 [SMILES: O=C(O)COCCOCCOCCCl]

A mixture of 2-(2-(2-(2-chloroethoxy)ethoxy)ethoxy)ethan-1-ol (1.023 g, 4.8 mmol) in acetone (10 mL) was being stirred at room temperature when 2 mL of a 2.67 M Jones reagent was added dropwise over 15 min. The reaction was stirred for an additional 30 min followed by addition of 3 drops of 2-propanol. Water was added to the mixture followed by removal of the acetone in vacuo. Saturated NaCl solution was added, and the aqueous mixture was extracted with CHCl₃. The organic layers were combined and dried with anhydrous MgSO₄. The solvent was removed in vacuo, and the product was further purified with silica gel column chromatography (DCM:MeOH=8: 1-6:1 gradient elution) to give the product (765 mg, 70%).

### Step 2: 2-(2-(2-(2-chloroethoxy)ethoxy)ethoxy)acetyl chloride

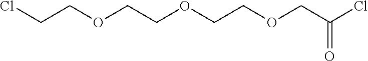 [SMILES: O=C(Cl)COCCOCCOCCCl]

2-(2-(2-(2-chloroethoxy)ethoxy)ethoxy)acetic acid (765 mg, 3.37 mmol) was combined with thionyl chloride(5 ml) at ambient temperature and 3 drops of DMF were added. The mixture was stirred for 16 h at 60° C. Then it was evaporated to afford the product (crude).

### Step 3: 2-(2-(2-(2-chloroethoxy)ethoxy)ethoxy)-N-(2-(2, 6-dioxopiperidin-3-yl)-1, 3-dioxoisoindolin-5-yl)acetamide

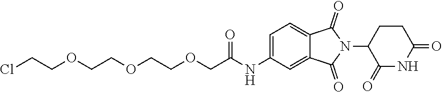 [SMILES: O=C1CCC(N2C(=O)c3ccc(NC(=O)COCCOCCOCCCl)cc3C2=O)C(=O)N1]

2-(2-(2-(2-chloroethoxy)ethoxy)ethoxy)acetyl chloride (826 mg, 3.37 mmol, 1.8 eq) was dissolved in THF (10 mL). To this solution was added 5-amino-2-(2, 6-dioxopiperidin-3-yl)isoindoline-1, 3-dione (500 mg, 1.83 mmol). The resulting suspension was heated to reflux for 4 hours. The solvent was evaporated in vacuo and the residue was purified with silica gel column chromatography (PE:EA=1:1˜1:4 gradient elution) to give the product (716 mg, 81%). [M+H]⁺=481.9.

### Step 4: N-(2-(2, 6-dioxopiperidin-3-yl)-1, 3-dioxoisoindolin-5-yl)-2-(2-(2-(2-iodoethoxy) ethoxy)ethoxy)acetamide

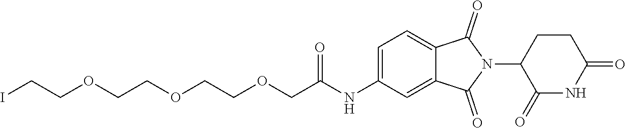 [SMILES: O=C1CCC(N2C(=O)c3ccc(NC(=O)COCCOCCOCCI)cc3C2=O)C(=O)N1]

To a solution of 2-(2-(2-(2-chloroethoxy)ethoxy)ethoxy)-N-(2-(2, 6-dioxopiperidin-3-yl)-1, 3-dioxoisoindolin-5-yl)acetamide (716 mg, 1.49 mmol) in acetone (20 mL) was added NaI (1.11 g, 7.4 mmol, 4.8 eq) was added. The reaction mixture was stirred at refluxed temperature for 4 h, then the solvent was removed under vacuum and the crude product was dissolved in EtOAc. An aqueous solution of Na₂SO₃ was added, and the organic layer was separated, washed with water, and dried. The solid was filtered off and the volatiles were evaporated under vacuum to give the product (668 mg, 78%). [M+H]⁺=573.8.

### Step 5: (7S)-7-(1-(2-(2-(2-(2-((2-(2, 6-dioxopiperidin-3-yl)-1, 3-dioxoisoindolin-5-yl)amino)-2-oxoethoxy)ethoxy)ethoxy)ethyl)piperidin-4-yl)-2-(4-phenoxyphenyl)-4, 5, 6, 7-tetrahydropyrazolo[1,5-a]pyrimidine-3-carboxamide

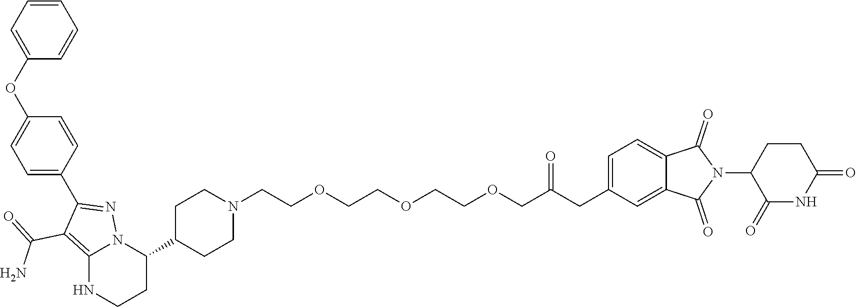 [SMILES: NC(=O)c1c(-c2ccc(Oc3ccccc3)cc2)nn2c1NCC[C@H]2C1CCN(CCOCCOCCOCC(=O)Cc2ccc3c(c2)C(=O)N(C2CCC(=O)NC2=O)C3=O)CC1]

To a solution of (S)-2-(4-phenoxyphenyl)-7-(piperidin-4-yl)-4, 5, 6, 7-tetrahydropyrazolo[1, 5-a]pyrimidine-3-carboxamide (72.9 mg, 0.17 mmol) and TEA (0.12 mL, 0.51 mmol) in DMF (5.8 mL) was added N-(2-(2, 6-dioxopiperidin-3-yl)-1, 3-dioxoisoindolin-5-yl)-2-(2-(2-(2-iodoethoxy) ethoxy)ethoxy)acetamide (100 mg, 0.17 mmol) and the resulting solution was stirred for 16 h at room temperature. The solvent was evaporated and the residue was further purified with pre-HPLC to give the title product (52.78 mg, 35%). ¹H NMR (400 MHz, CDCl₃) δ_{H} 11.86 (brs, 1H), 9.41 (brs, 2H), 8.72-8.29 (m, 1H), 7.88-7.78 (m, 2H), 7.53-7.33 (m, 3H), 7.21-6.94 (m, 5H), 6.55 (brs, 2H), 5.49 (brs, 1H), 5.00-4.82 (m, 1H), 4.16-4.12 (m, 2H), 4.04-3.01 (m, 19H), 2.94-2.45 (m, 5H), 2.14-1.63 (m, 6H).; [M+H]⁺=862.9.

### Example 24: (7S)-7-(1-(2-(2-(2-((2-(2, 6-dioxopiperidin-3-yl)-1, 3-dioxoisoindolin-5-yl)amino)-2-oxoethoxy)ethoxy)ethyl)piperidin-4-yl)-2-(4-phenoxyphenyl)-4, 5, 6, 7-tetrahydropyrazolo[1,5-a]pyrimidine-3-carboxamide

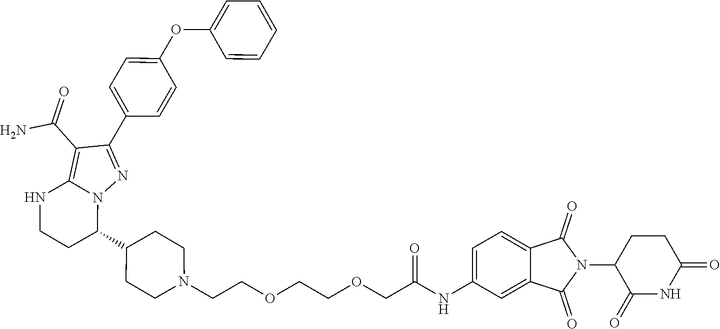 [SMILES: NC(=O)c1c(-c2ccc(Oc3ccccc3)cc2)nn2c1NCC[C@H]2C1CCN(CCOCCOCC(=O)Nc2ccc3c(c2)C(=O)N(C2CCC(=O)NC2=O)C3=O)CC1]

The titled compound was synthesized in the procedures similar to Example 23. ¹H NMR (400 MHz, DMSO) δ_{H} 11.12 (s, 1H), 10.34 (s, 1H), 8.05 (d, J=8.0 Hz, 1H), 8.30 (s, 1H), 7.89 (d, J=8.0 Hz, 1H), 7.40-7.50 (m, 4H), 7.04-7.18 (m, 5H), 6.63 (s, 1H), 5.12-5.15 (m, 1H), 4.18 (s, 2H), 3.94-4.04 (m, 2H), 3.52-3.69 (m, 6H), 3.26-3.29 (m, 1H), 2.86-2.90 (m, 3H), 2.44-2.61 (m, 3H), 1.87-2.06 (m, 6H), 1.57-1.60 (m, 1H), 1.16-1.42 (m, 6H); [M+H]⁺=819.6.

### Example 25: (7S)-7-(1-(2-((8-(2-((2-(2, 6-dioxopiperidin-3-yl)-1-oxoisoindolin-4-yl)oxy)acetamido)octyl)oxy)acetyl)piperidin-4-yl)-2-(4-phenoxyphenyl)-4, 5, 6, 7-tetrahydropyrazolo[1,5-a] pyrimidine-3-carboxamide

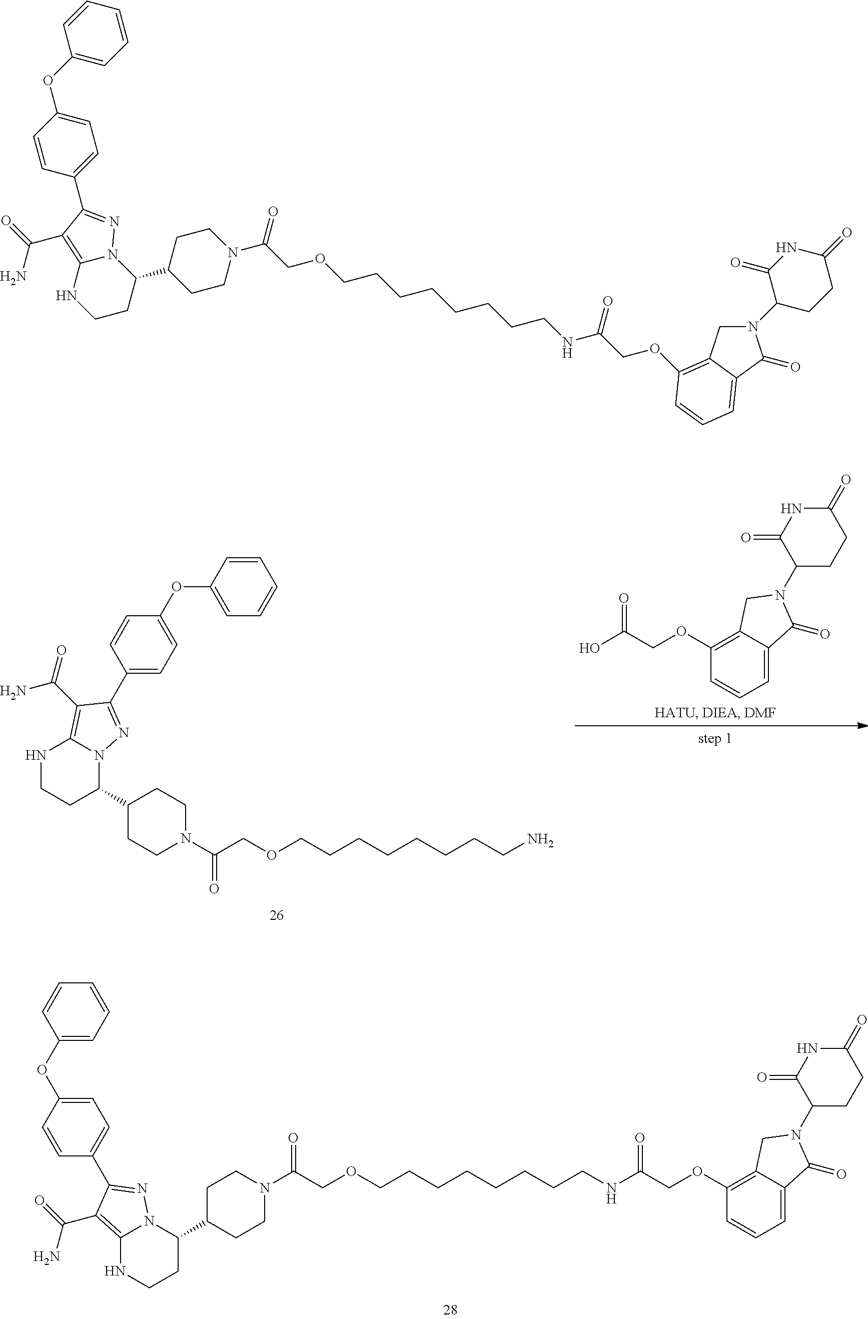 [SMILES: NC(=O)c1c(-c2ccc(Oc3ccccc3)cc2)nn2c1NCC[C@H]2C1CCN(C(=O)COCCCCCCCCNC(=O)COc2cccc3c2CN(C2CCC(=O)NC2=O)C3=O)CC1 ; NCCCCCCCCOCC(=O)N1CCC([C@@H]2CCNc3c(C(N)=O)c(-c4ccc(Oc5ccccc5)cc4)nn32)CC1 ; O=C(O)COc1cccc2c1CN(C1CCC(=O)NC1=O)C2=O ; NC(=O)c1c(-c2ccc(Oc3ccccc3)cc2)nn2c1NCC[C@H]2C1CCN(C(=O)COCCCCCCCCNC(=O)COc2cccc3c2CN(C2CCC(=O)NC2=O)C3=O)CC1]

### Step 1: (7S)-7-(1-(2-((8-(2-((2-(2, 6-dioxopiperidin-3-yl)-1-oxoisoindolin-4-yl)oxy)acetamido)octyl)oxy)acetyl)piperidin-4-yl)-2-(4-phenoxyphenyl)-4, 5, 6, 7-tetrahydropyrazolo[1, 5-a]pyrimidine-3-carboxamide

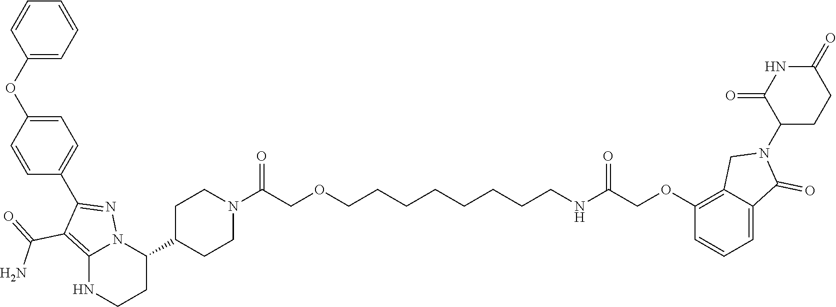 [SMILES: NC(=O)c1c(-c2ccc(Oc3ccccc3)cc2)nn2c1NCC[C@H]2C1CCN(C(=O)COCCCCCCCCNC(=O)COc2cccc3c2CN(C2CCC(=O)NC2=O)C3=O)CC1]

To a solution of the compound 2-((2-(2, 6-dioxopiperidin-3-yl)-1-oxoisoindolin-4-yl)oxy)acetic acid (0.0212 g, 0.066 mmol) and (S)-7-(1-(2-((8-aminooctyl)oxy)acetyl)piperidin-4-yl)-2-(4-phenoxyphenyl)-4, 5, 6, 7-tetrahydropyrazolo[1,5-a]pyrimidine-3-carboxamide (0.040 g, 0.066 mmol) in dry DMF (2 ml) at 0° C., DIEA (55 ul, 5 eq) then HATU(0.0303 g, 0.08 mmol, 1.20 eq) was added. The reaction was stirred at room temperature for 12 hours. Water (10 ml) was added to quench the reaction, and the reaction mixture was then extracted with EA (10 ml*3). The combined organic layer was washed with brine (10 ml*3), dried over Na₂SO₄, filtered, concentrated to dryness and purified with Pre-HPLC to afford the product (24.62 mg, 41%). ¹H NMR (400 MHz, DMSO) δ_{H} 11.01 (s, 1H), 8.05 (s, 1H), 7.42 (ddd, J=34.3, 22.8, 8.0 Hz, 6H), 7.12 (ddd, J=24.3, 17.0, 8.0 Hz, 6H), 6.67 (s, 1H), 5.13 (dd, J=13.2, 5.0 Hz, 1H), 4.61 (s, 2H), 4.40 (dd, J=46.3, 17.5 Hz, 3H), 4.06 (dd, J=32.4, 12.7 Hz, 3H), 3.83 (d, J=11.0 Hz, 1H), 3.37 (d, J=6.3 Hz, 2H), 3.10 (d, J=6.3 Hz, 2H), 2.98-2.87 (m, 3H), 2.65 (d, J=20.2 Hz, 3H), 2.42 (d, J=13.7 Hz, 1H), 2.23 (s, 1H), 1.98 (dd, J=24.7, 18.6 Hz, 3H), 1.68 (s, 1H), 1.35-1.55 (m, 6H), 1.10-1.30 (m, 10H); [M+H]⁺=902.9.

### Example 26: (7S)-7-(1-(2-((8-((2-(2, 6-dioxopiperidin-3-yl)-1, 3-dioxoisoindolin-4-yl)amino)octyl)oxy)acetyl)piperidin-4-yl)-2-(4-phenoxyphenyl)-4, 5, 6, 7-tetrahydropyrazolo[1, 5-a]pyrimidine-3-carboxamide

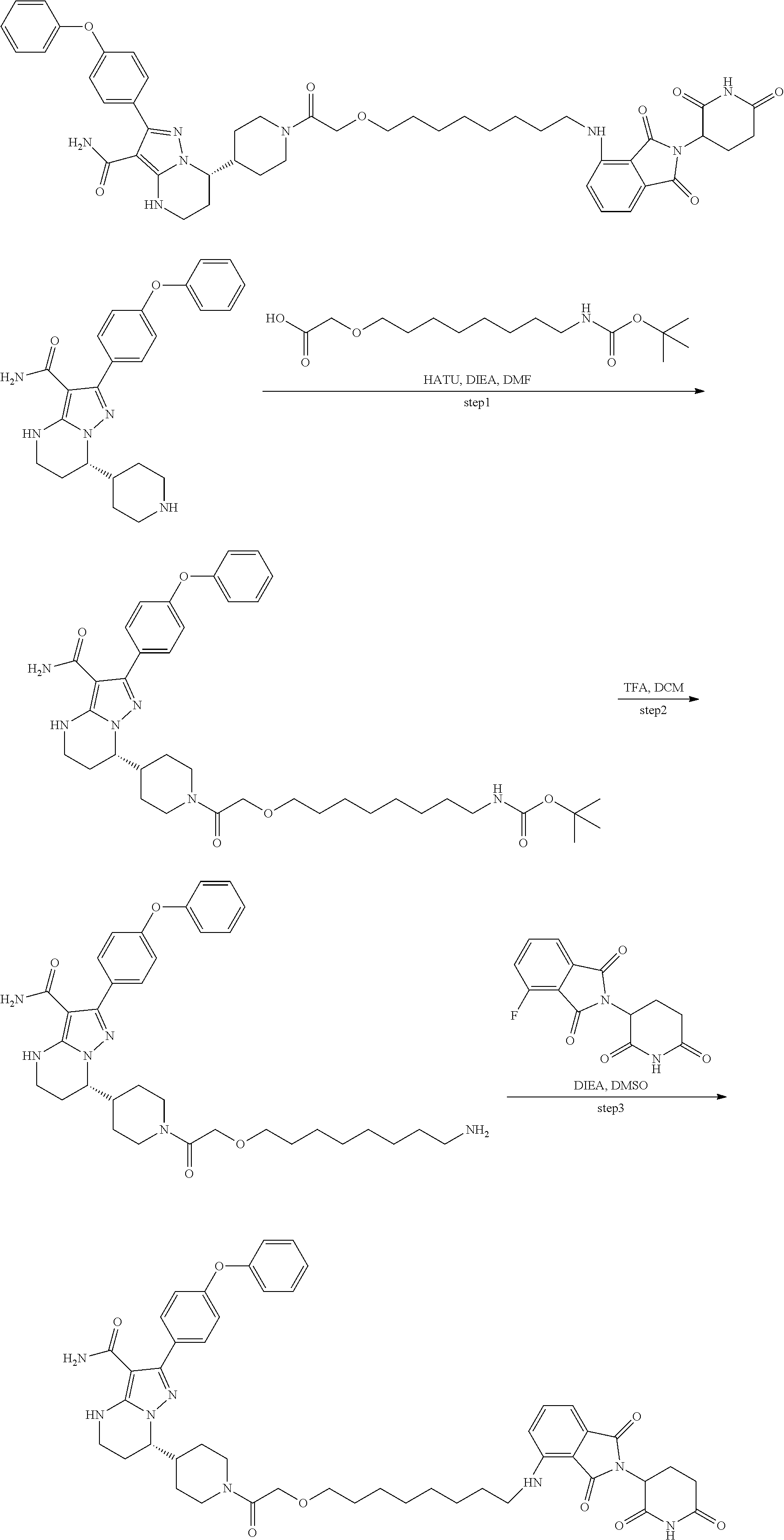

### Step 1: tert-butyl (S)-(8-(2-(4-(3-carbamoyl-2-(4-phenoxyphenyl)-4, 5, 6, 7-tetrahydropyrazolo[1, 5-a]pyrimidin-7-yl)piperidin-1-yl)-2-oxoethoxy)octyl)carbamate

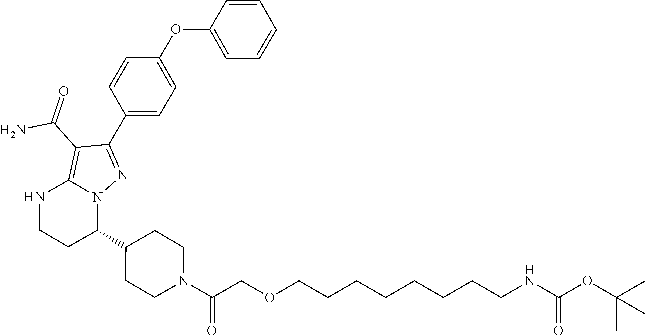 [SMILES: CC(C)(C)OC(=O)NCCCCCCCCOCC(=O)N1CCC([C@@H]2CCNc3c(C(N)=O)c(-c4ccc(Oc5ccccc5)cc4)nn32)CC1]

To a solution of the compound 2-((8-((tert-butoxycarbonyl)amino)octyl)oxy)acetic acid (0.8 g, 2.64 mmol) dissolved in DMF (15 ml) were added (S)-2-(4-phenoxyphenyl)-7-(piperidin-4-yl)-4, 5, 6, 7-tetrahydropyrazolo[1,5-a]pyrimidine-3-carboxamide (1.1 g, 2.64 mmol,) and DIEA (1.70 g, 13.2 mmol). Then HATU (1.21 g, 3.164 mmol) was added in one portion. The mixture was stirred at room temperature overnight. Then EA (150 mL) was added, and the mixture was washed with brine 50 ml for 3 times. The organic layer was separated, combined and dried over Na₂SO₄, then filtered and concentrated to dryness. The residue was purified with column chromatography (DCM/MeOH=10:1) to afford the product (0.92 g, 50%). [M+H]⁺=703.0.

### Step 2: (S)-7-(1-(2-(8-aminooctyl)oxy)acetyl)piperidin-4-yl)-2-(4-phenoxyphenyl)-4, 5, 6, 7-tetrahydropyrazolo[1,5-a]pyrimidine-3-carboxamide

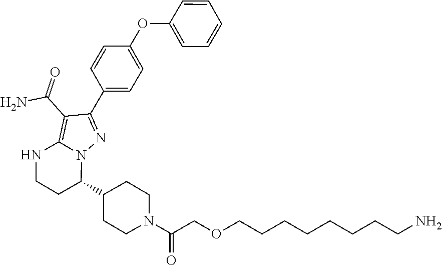 [SMILES: NCCCCCCCCOCC(=O)N1CCC([C@@H]2CCNc3c(C(N)=O)c(-c4ccc(Oc5ccccc5)cc4)nn32)CC1]

To a solution of tert-butyl (S)-(8-(2-(4-(3-carbamoyl-2-(4-phenoxyphenyl)-4, 5, 6, 7-tetrahydropyrazolo[1,5-a]pyrimidin-7-yl)piperidin-1-yl)-2-oxoethoxy)octyl) carbamate (0.92 g, 1.31 mmol, 1.00 eq) in DCM (16 ml), TFA (8 ml) was added. The mixture was stirred at 25° C. for 1 hour. The solution was concentrated to dryness, purified with column chromatography (DCM/MeOH=10:1, 1% ammonia water) to afford the product (0.61 g, 77%). [M+H]⁺=603.0.

### Step 3: (7S)-7-(1-(2-((8-((2-(2, 6-dioxopiperidin-3-yl)-1, 3-dioxoisoindolin-4-yl)amino)octyl)oxy)acetyl)piperidin-4-yl)-2-(4-phenoxyphenyl)-4, 5, 6, 7-tetrahydropyrazolo[1, 5-a]pyrimidine-3-carboxamide

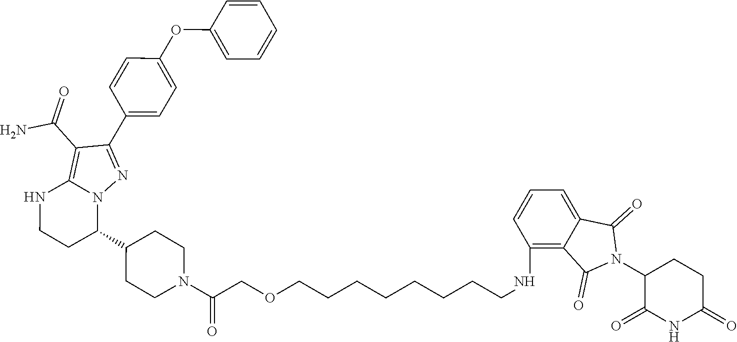 [SMILES: NC(=O)c1c(-c2ccc(Oc3ccccc3)cc2)nn2c1NCC[C@H]2C1CCN(C(=O)COCCCCCCCCNc2cccc3c2C(=O)N(C2CCC(=O)NC2=O)C3=O)CC1]

To a solution of (S)-7-(1-(2-((8-aminooctyl)oxy)acetyl)piperidin-4-yl)-2-(4-phenoxyphenyl)-4, 5, 6, 7-tetrahydropyrazolo[1,5-a]pyrimidine-3-carboxamide (0.1 g, 0.166 mmol) and 2-(2, 6-dioxopiperidin-3-yl)-4-fluoroisoindoline-1, 3-dione (0.051 g, 0.182 mmol) in DMSO (2 ml) was added DIEA (0.075 g, 0.581 mmol). The mixture was stirred at 120° C. under nitrogen protection for 3 hours, then at 110° C. overnight. The mixture was dissolved in EA (20 ml), washed with brine (10 ml*3) and concentrated to dryness. The crude residue was purified with pre-HPLC to give the product (20.18 mg, 14.2%). ¹H NMR (400 MHz, DMSO) δ_{H} 11.09 (s, 1H), 7.57 (t, J=7.8 Hz, 1H), 7.50 (d, J=8.4 Hz, 2H), 7.41 (t, J=7.8 Hz, 2H), 7.17 (t, J=7.3 Hz, 1H), 7.13-6.96 (m, 6H), 6.67 (s, 1H), 6.51 (t, J=5.5 Hz, 1H), 5.05 (dd, J=12.8, 5.2 Hz, 1H), 4.37 (d, J=10.9 Hz, 1H), 4.06 (dd, J=35.8, 12.4 Hz, 3H), 3.84 (d, J=11.7 Hz, 1H), 3.28-3.42 (m, 5H), 2.97-2.81 (m, 2H), 2.69-2.53 (m, 2H), 2.22 (s, 1H), 2.07-1.84 (m, 3H), 1.68 (s, 1H), 1.60-1.43 (m, 5H), 1.41-1.13 (m, 12H); [M+H]⁺=858.9.

### Example 27: (7S)-7-(1-(14-((2-(2, 6-dioxopiperidin-3-yl)-1, 3-dioxoisoindolin-4-yl)amino)-3, 6, 9, 12-tetraoxatetradecanoyl)piperidin-4-yl)-2-(4-phenoxyphenyl)-4, 5, 6, 7-tetrahydropyrazolo[1, 5-a]pyrimidine-3-carboxamide

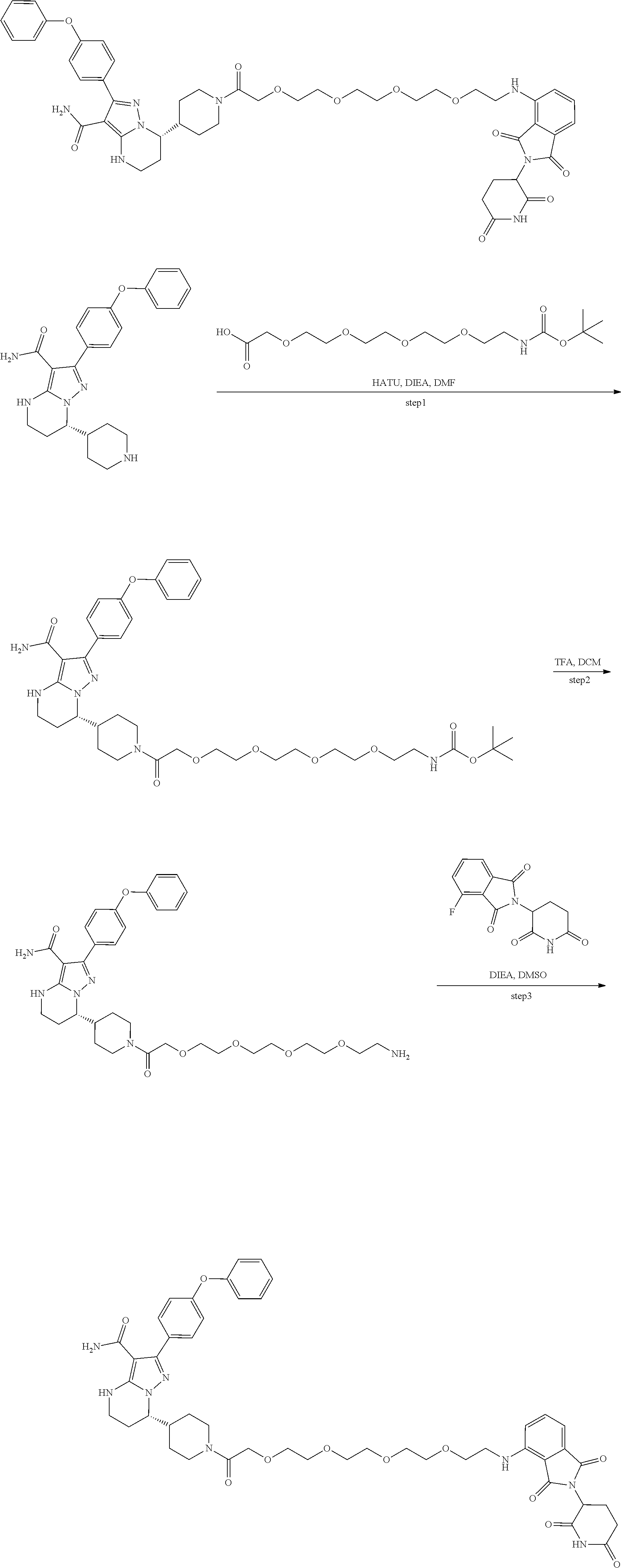

### Step 1: tert-butyl (S)-(14-(4-(3-carbamoyl-2-(4-phenoxyphenyl)-4, 5, 6, 7-tetrahydropyrazolo[1, 5-a]pyrimidin-7-yl)piperidin-1-yl)-14-oxo-3, 6, 9, 12-tetraoxatetradecyl)carbamate

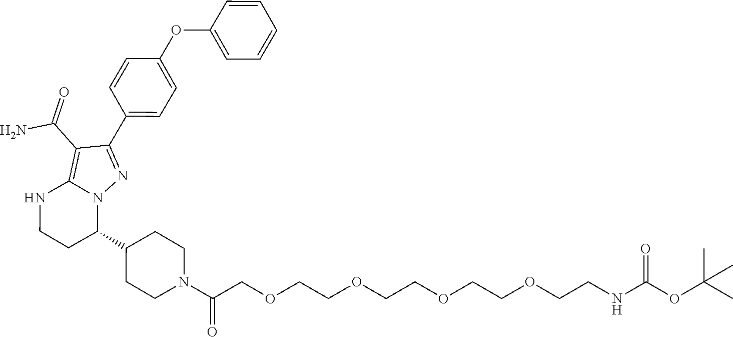 [SMILES: CC(C)(C)OC(=O)NCCOCCOCCOCCOCC(=O)N1CCC([C@@H]2CCNc3c(C(N)=O)c(-c4ccc(Oc5ccccc5)cc4)nn32)CC1]

To a solution of the compound 2, 2-dimethyl-4-oxo-3, 8, 11, 14, 17-pentaoxa-5-azanonadecan-19-oic acid (1.0 g, 2.846 mmol) dissolved in DMF (15 ml) were added (S)-2-(4-phenoxyphenyl)-7-(piperidin-4-yl)-4, 5, 6, 7-tetrahydropyrazolo[1,5-a]pyrimidine-3-carboxamide (1.19 g, 2.846 mmol) and DIEA (1.84 g, 14.32 mmol). Then HATU (1.30 g, 3.415 mmol) was added in one portion. The mixture was stirred at room temperature overnight. Then EA (150 mL) was added, and the mixture was washed with brine 50 ml*3 times. The organic layer was separated, combined, dried over Na₂SO₄, then filtered and concentrated to dryness. The residue was purified with column chromatography (DCM/MeOH=10:1) to afford the product (2.10 g, 98%). [M+H]⁺=751.0.

### Step 2: (S)-7-(1-(14-amino-3, 6, 9, 12-tetraoxatetradecanoyl)piperidin-4-yl)-2-(4-phenoxyphenyl)-4, 5, 6, 7-tetrahydropyrazolo[1, 5-a]pyrimidine-3-carboxamide

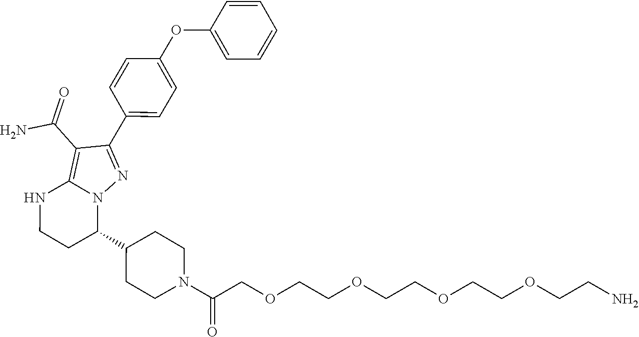 [SMILES: NCCOCCOCCOCCOCC(=O)N1CCC([C@@H]2CCNc3c(C(N)=O)c(-c4ccc(Oc5ccccc5)cc4)nn32)CC1]

To a solution of tert-butyl (S)-(14-(4-(3-carbamoyl-2-(4-phenoxyphenyl)-4, 5, 6, 7-tetrahydropyrazolo[1,5-a]pyrimidin-7-yl)piperidin-1-yl)-14-oxo-3, 6, 9, 12-tetraoxatetradecyl)carbamate (2.10 g, 2.80 mmol) in DCM (16 ml), TFA (8 ml) was added. The mixture was stirred at 25° C. for 1 hour. The solution was concentrated to dryness, purified with column chromatography (DCM/MeOH=10:1, 1% ammonia water) to afford the product (1.30 g, 71%). [M+H]⁺=651.0.

### Step 3: (7S)-7-(1-(14-((2-(2, 6-dioxopiperidin-3-yl)-1, 3-dioxoisoindolin-4-yl)amino)-3, 6, 9, 12-tetraoxatetradecanoyl)piperidin-4-yl)-2-(4-phenoxyphenyl)-4, 5, 6, 7-tetrahydropyrazolo[1, 5-a]pyrimidine-3-carboxamide

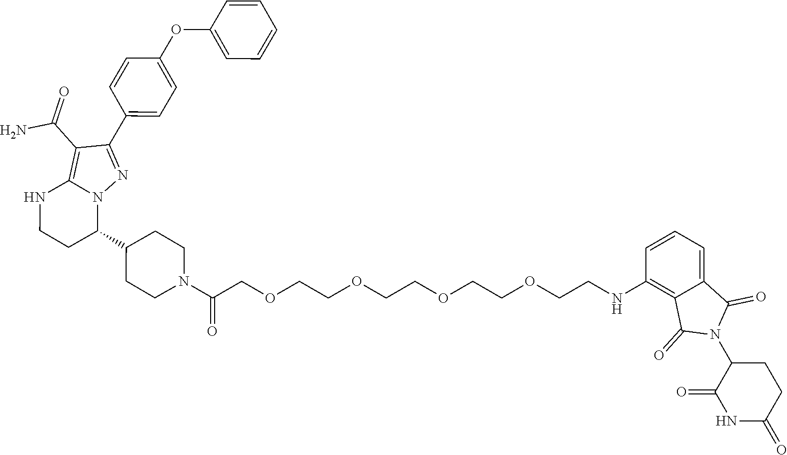 [SMILES: NC(=O)c1c(-c2ccc(Oc3ccccc3)cc2)nn2c1NCC[C@H]2C1CCN(C(=O)COCCOCCOCCOCCNc2cccc3c2C(=O)N(C2CCC(=O)NC2=O)C3=O)CC1]

To a solution of (S)-7-(1-(14-amino-3, 6, 9, 12-tetraoxatetradecanoyl)piperidin-4-yl)-2-(4-phenoxyphenyl)-4, 5, 6, 7-tetrahydropyrazolo[1,5-a]pyrimidine-3-carboxamide (0.1 g, 0.154 mmol) and 2-(2, 6-dioxopiperidin-3-yl)-4-fluoroisoindoline-1, 3-dione (0.047 g, 0.169 mmol) in DMSO (2 ml) was added DIEA (0.060 g, 0.462 mmol). The mixture was stirred at 110° C. under nitrogen protection for 12 hours. The mixture was dissolved in EA (20 ml), washed with brine (10 ml*3) and concentrated to dryness. The crude residue was purified with pre-HPLC to give the product (41.63 mg, 29.8%). ¹H NMR δ_{H} 11.09 (s, 1H), 7.60-7.54 (m, 1H), 7.50 (d, J=8.6 Hz, 2H), 7.41 (dd, J=8.5, 7.5 Hz, 2H), 7.15 (dd, J=16.9, 8.1 Hz, 2H), 7.05 (dd, J=16.8, 8.0 Hz, 5H), 6.67 (s, 1H), 6.60 (t, J=5.8 Hz, 1H), 5.05 (dd, J=12.9, 5.3 Hz, 1H), 4.36 (s, 1H), 4.18-3.96 (m, 3H), 3.80 (s, 1H), 3.60 (t, J=5.4 Hz, 2H), 3.56-3.42 (m, 14H), 3.29 (s, 2H), 2.88 (s, 2H), 2.62-2.52 (m, 2H), 2.22 (s, 1H), 2.07-1.85 (m, 3H), 1.68 (s, 1H), 1.50 (d, J=16.5 Hz, 1H), 1.09-1.38 (m, 3H); [M+H]⁺=906.9.

### Example 28: (7S)-7-(1-(2-(2-(2-(3-(4-(2-(2, 6-dioxopiperidin-3-yl)-1, 3-dioxoisoindolin-5-yl)piperazin-1-yl)propanamido)ethoxy)ethoxy)acetyl)piperidin-4-yl)-2-(4-phenoxyphenyl)-4, 5, 6, 7-tetrahydropyrazolo[1,5-a]pyrimidine-3-carboxamide

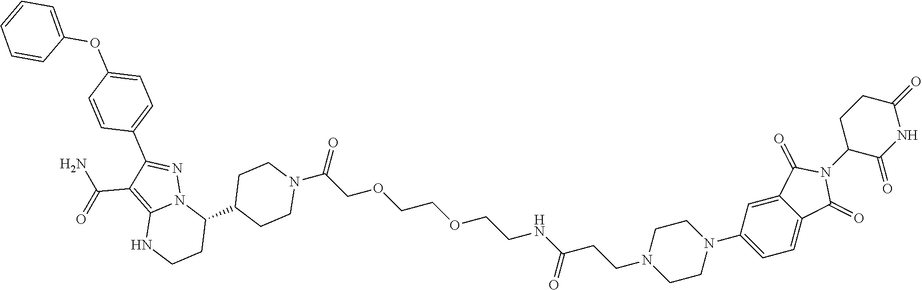

The titled compound was synthesized in the procedures similar to Example 23. ¹H NMR (400 MHz, DMSO) δ_{H} 11.08 (s, 1H), 8.05 (s, 1H), 7.66 (d, J=8.0 Hz, 1H), 7.50 (d, J=8.0 Hz, 2H), 7.41 (t, J=8.0 Hz, 2H), 7.34 (s, 1H), 7.24 (d, J=8.0 Hz, 1H), 7.17 (t, J=8.0 Hz, 1H), 7.06 (t, J=8.0 Hz, 4H), 6.68 (s, 1H), 5.07 (dd, J=12.0, 4.0 Hz, 1H), 4.37 (d, J=8.0 Hz, 1H), 4.11 (dd, J=32.0, 12.0 Hz, 2H), 4.00 (s, 1H), 3.79 (s, 1H), 3.52 (s, 5H), 3.41 (s, 6H), 3.29-3.24 (m, 2H), 3.23-3.15 (m, 2H), 2.93-2.81 (m, 2H), 2.64-2.52 (m, 8H), 2.31-2.15 (m, 3H), 2.07-1.83 (m, 3H), 1.62-1.75 (m, 1H), 1.59-1.46 (m, 1H), 1.42-1.10 (m, 4H); [M+H]⁺=958.8.

### Example 29: (7S)-7-(1-(1-((2-(2, 6-dioxopiperidin-3-yl)-1-oxoisoindolin-4-yl)oxy)-2-oxo-6, 9, 12, 15-tetraoxa-3-azaheptadecan-17-oyl)piperidin-4-yl)-2-(4-phenoxyphenyl)-4, 5, 6, 7-tetrahydropyrazolo[1,5-a]pyrimidine-3-carboxamide

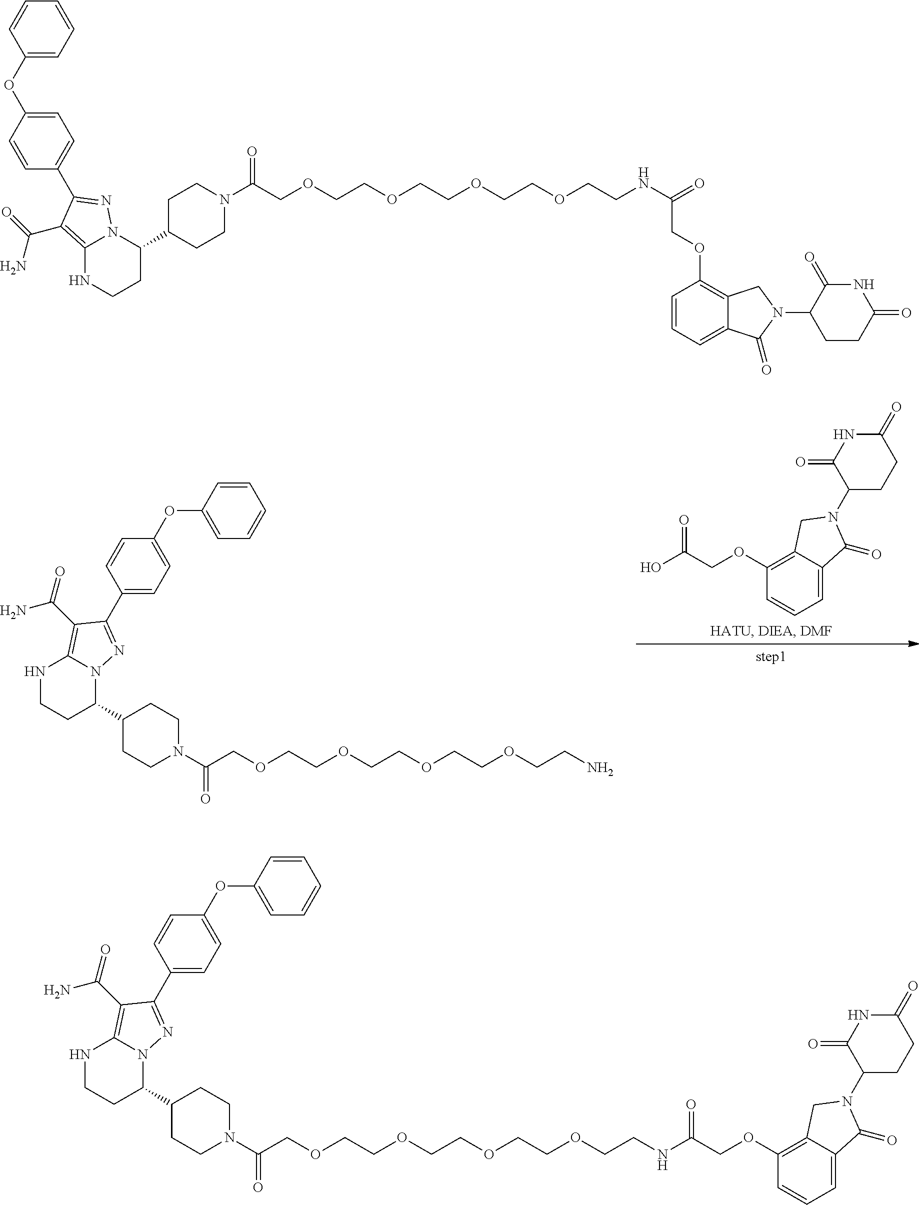 [SMILES: NC(=O)c1c(-c2ccc(Oc3ccccc3)cc2)nn2c1NCC[C@H]2C1CCN(C(=O)COCCOCCOCCOCCNC(=O)COc2cccc3c2CN(C2CCC(=O)NC2=O)C3=O)CC1 ; NCCOCCOCCOCCOCC(=O)N1CCC([C@@H]2CCNc3c(C(N)=O)c(-c4ccc(Oc5ccccc5)cc4)nn32)CC1 ; O=C(O)COc1cccc2c1CN(C1CCC(=O)NC1=O)C2=O ; NC(=O)c1c(-c2ccc(Oc3ccccc3)cc2)nn2c1NCC[C@H]2C1CCN(C(=O)COCCOCCOCCOCCNC(=O)COc2cccc3c2CN(C2CCC(=O)NC2=O)C3=O)CC1]

### Step 1: (7S)-7-(1-(1-((2-(2, 6-dioxopiperidin-3-yl)-1-oxoisoindolin-4-yl)oxy)-2-oxo-6, 9, 12, 15-tetraoxa-3-azaheptadecan-17-oyl)piperidin-4-yl)-2-(4-phenoxyphenyl)-4, 5, 6, 7-tetrahydropyrazolo[1,5-a]pyrimidine-3-carboxamide

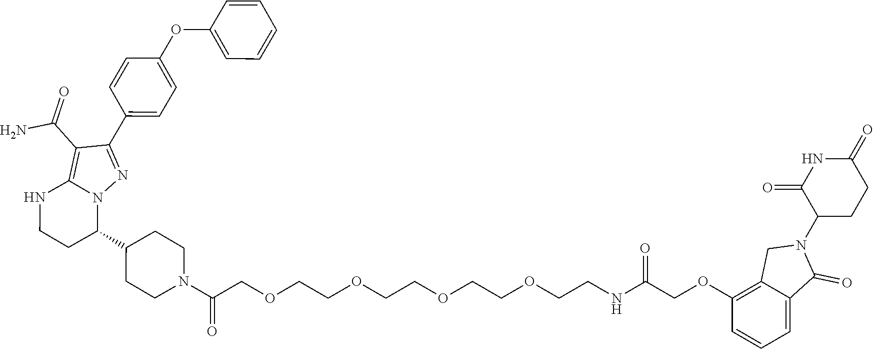 [SMILES: NC(=O)c1c(-c2ccc(Oc3ccccc3)cc2)nn2c1NCC[C@H]2C1CCN(C(=O)COCCOCCOCCOCCNC(=O)COc2cccc3c2CN(C2CCC(=O)NC2=O)C3=O)CC1]

To a solution of the compound 2-((2-(2, 6-dioxopiperidin-3-yl)-1-oxoisoindolin-4-yl)oxy)acetic acid (0.020 g, 0.0615 mmol) and (S)-7-(1-(14-amino-3, 6, 9, 12-tetraoxatetradecanoyl)piperidin-4-yl)-2-(4-phenoxyphenyl)-4, 5, 6, 7-tetrahydropyrazolo[1, 5-a]pyrimidine-3-carboxamide (0.040 g, 0.0615 mmol) in dry DMF (2 ml) at 0° C., DIEA (56 mg, 0.43 mmol), then HATU(0.028 g, 0.0737 mmol), was added. The reaction was stirred at room temperature for 12 hours. Water (10 ml) was added to quench the reaction, then the reaction mixture was extracted with EA (10 ml*3). The combined organic layer was washed with brine (10 ml*3), dried over Na₂SO₄, filtered, concentrated to dryness and purified with pre-HPLC to afford the product (19.98 mg, 34%). ¹H NMR (400 MHz, DMSO) δ_{H} 11.01 (s, 1H), 8.12 (t, J=5.7 Hz, 1H), 7.52-7.39 (m, 5H), 7.34 (d, J=7.4 Hz, 1H), 7.12 (ddd, J=30.2, 16.6, 8.1 Hz, 6H), 6.67 (s, 1H), 5.13 (dd, J=13.3, 4.9 Hz, 1H), 4.64 (s, 2H), 4.39 (dd, J=48.1, 17.6 Hz, 3H), 4.11 (d, J=16.7 Hz, 2H), 4.00 (s, 1H), 3.80 (s, 1H), 3.54-3.46 (m, 12H), 3.43 (t, J=5.9 Hz, 2H), 3.31-3.25 (m, 4H), 2.98-2.85 (m, 2H), 2.48-2.60 (m, 3H), 2.21 (s, 1H), 2.04-1.95 (m, 2H), 1.92 (s, 1H), 1.67 (s, 1H), 1.52 (s, 1H), 1.11-1.38 (m, 2H); [M+H]⁺=950.9.

### Example 30: (7S)-7-(1-(5-(2-(2-(4-(2-(2, 6-dioxopiperidin-3-yl)-1, 3-dioxoisoindolin-5-yl)piperazin-1-yl)acetamido)ethoxy)pentanoyl)piperidin-4-yl)-2-(4-phenoxyphenyl)-4, 5, 6, 7-tetrahydropyrazolo[1,5-a] pyrimidine-3-carboxamide

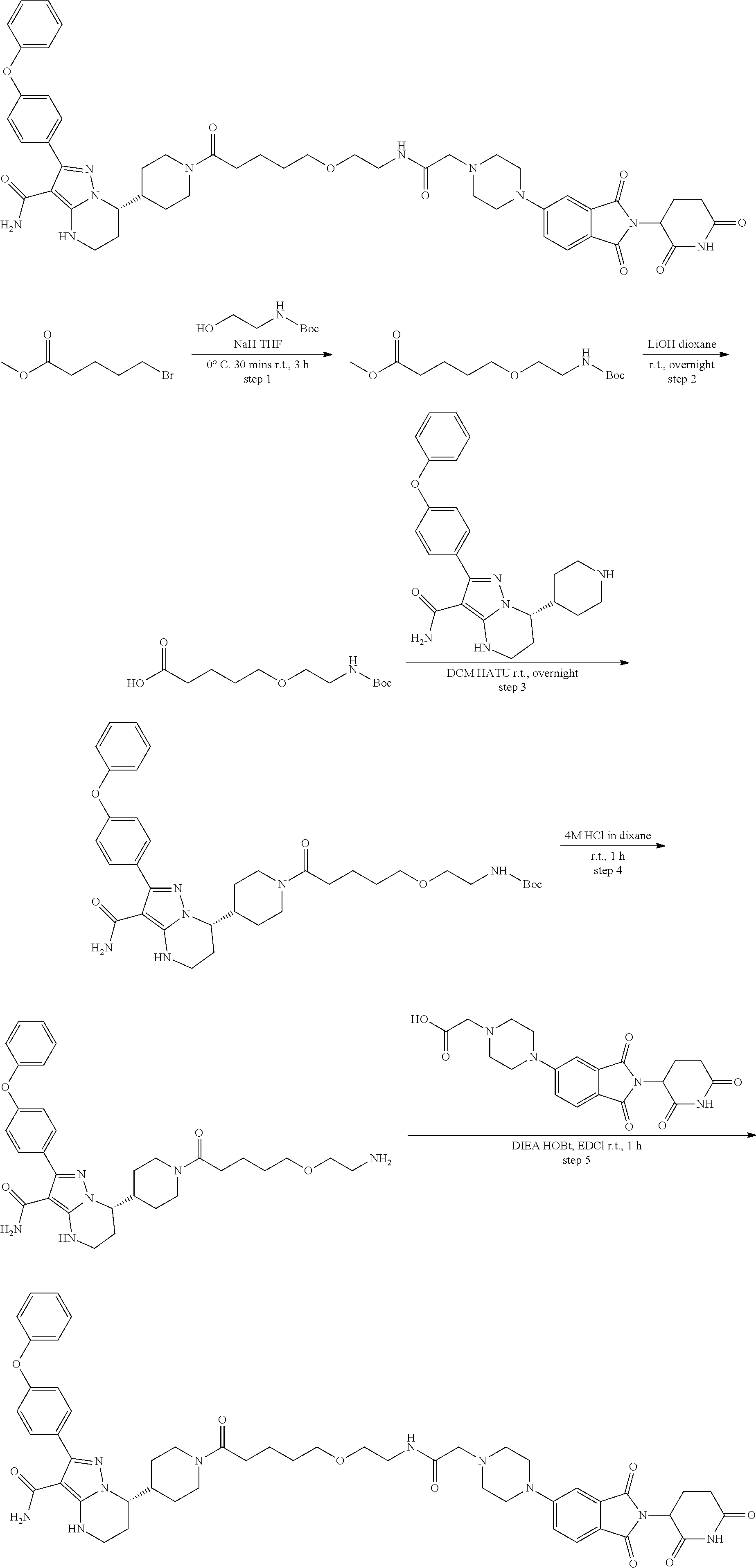

### Step 1: methyl 5-(2-((tert-butoxycarbonyl)amino)ethoxy)pentanoate

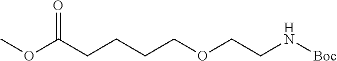 [SMILES: COC(=O)CCCCOCCNC(=O)OC(C)(C)C]

To a solution of tert-butyl (2-hydroxyethyl)carbamate (4 g, 0.025 mol) in dry THF (50 mL), was added sodium hydride (1.5 g, 0.0375 mol) with stirring in portions at 0° C. The reaction mixture was stirred at 0° C. for 30 min. Then methyl 5-bromopentanoate (4.39 g, 0.0225 mol, 0.9 eq) was added at 0° C. The mixture was stirred from 0° C. to rt and stirred at r.t. for 3 h. Then the mixture was quenched with 10 mL water and extracted with EA (150 mL*2). The organic phases were combined, dried over by anhydrous Na₂SO₄, filtered and concentrated in vacuo to give desired product (1.6 g, 23%), which was used in the next step without further purification. [M+H]⁺=276.0.

### Step 2: 5-(2-((tert-butoxycarbonyl)amino)ethoxy)pentanoic Acid

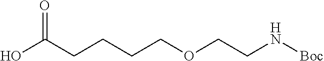 [SMILES: CC(C)(C)OC(=O)NCCOCCCCC(=O)O]

To a solution of methyl 5-(2-((tert-butoxycarbonyl)amino)ethoxy)pentanoate (1.6 g, 0.00575 mol) in 1, 4-dioxane (50 mL), was added lithium hydroxide (0.725 g, 0.0172 mmol) with stirring, and then water (50 mL). The reaction mixture was stirred at R.T. overnight. After the reaction was completed, the mixture was concentrated in vacuo to remove dioxane. The residue was extracted with EA (50 mL*2), and the pH value of the combined aqueous phase was adjusted to 5-6 with hydrochloric acid (1 mol/L). Then the aqueous phase was extracted with ethyl acetate (50 mL*2), organic phase was combined and dried over anhydrous Na₂SO₄, suction-filtered, then concentrated in vacuo to give the desired product (200 mg, 13%), which was used in the next step without further purification. [M+H]⁺=262.0.

### Step 3: tert-butyl (S)-(2-((5-(4-(3-carbamoyl-2-(4-phenoxyphenyl)-4, 5, 6, 7-tetrahydropyrazolo[1, 5-a]pyrimidin-7-yl)piperidin-1-yl)-5-oxopentyl)oxy)ethyl) carbamate

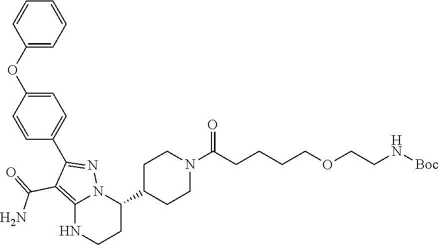 [SMILES: CC(C)(C)OC(=O)NCCOCCCCC(=O)N1CCC([C@@H]2CCNc3c(C(N)=O)c(-c4ccc(Oc5ccccc5)cc4)nn32)CC1]

A mixture of (S)-2-(4-phenoxyphenyl)-7-(piperidin-4-yl)-4, 5, 6, 7-tetrahydropyrazolo[1, 5-a]pyrimidine-3-carboxamide (319 mg, 0.766 mmol), 5-(2-((tert-butoxycarbonyl)amino)ethoxy)pentanoic acid (200 mg, 0.766 mmol), HATU (582 mg, 1.532 mmol) and DIPEA (296 mg, 2.298 mmol) in DCM (10 mL) was stirred at R.T. overnight. After LCMS indicated that the reaction was completed, the reaction mixture was concentrated in vacuo. The residue was purified with silica gel column chromatography DCM:MeOH (100%:0% 90%:10%) to afford the desired product (100 mg, 19%). [M+H]⁺=661.0.

### Step 4: (S)-7-(1-(5-(2-aminoethoxy)pentanoyl)piperidin-4-yl)-2-(4-phenoxyphenyl)-4, 5, 6, 7-tetrahydropyrazolo[1, 5-a]pyrimidine-3-carboxamide hydrochloride

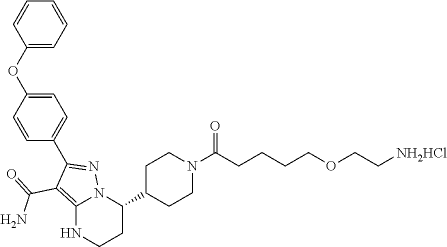

Tert-butyl (S)-(2-((5-(4-(3-carbamoyl-2-(4-phenoxyphenyl)-4, 5, 6, 7-tetrahydropyrazolo[1, 5-a]pyrimidin-7-yl)piperidin-1-yl)-5-oxopentyl)oxy)ethyl) carbamate (100 mg, 0.151 mmol) was dissolved in 4M HCl in dioxane (10 mL). Then the mixture was stirred at room temperature for 1 h. After determined the reaction to be complete by LCMS, the reaction mixture was concentrated in vacuo to afford the desired product (140 mg, crude), which was used in the next step without further purification. [M+H]⁺=561.0.

### Step 5: (7S)-7-(1-(5-(2-(2-(4-(2-(2, 6-dioxopiperidin-3-yl)-1, 3-dioxoisoindolin-5-yl)piperazin-1-yl)acetamido)ethoxy)pentanoyl)piperidin-4-yl)-2-(4-phenoxyphenyl)-4, 5, 6, 7-tetrahydropyrazolo[1,5-a]pyrimidine-3-carboxamide

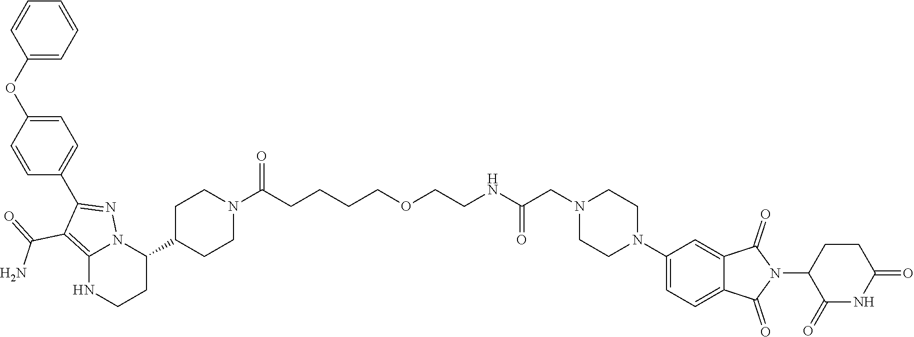 [SMILES: NC(=O)c1c(-c2ccc(Oc3ccccc3)cc2)nn2c1NCC[C@H]2C1CCN(C(=O)CCCCOCCNC(=O)CN2CCN(c3ccc4c(c3)C(=O)N(C3CCC(=O)NC3=O)C4=O)CC2)CC1]

A mixture of 2-(4-(2-(2, 6-dioxopiperidin-3-yl)-1, 3-dioxoisoindolin-5-yl)piperazin-1-yl)acetic acid (50 mg, 0.125 mmol), HOBT (35 mg, 0.260 mmol), EDCI (49 mg, 0.260 mmol) and DIPEA (67 mg, 0.52 mmol) in DCM (10 mL) was stirred at room temperature for 5 min. Then (S)-7-(1-(5-(2-aminoethoxy)pentanoyl)piperidin-4-yl)-2-(4-phenoxyphenyl)-4, 5, 6, 7-tetrahydropyrazolo[1,5-a]pyrimidine-3-carboxamide hydrochloride (62.3 mg, 0.104 mmol) was added. The mixture was stirred at room temperature overnight. After LCMS indicated that the reaction was completed, the reaction mixture was concentrated in vacuo. The residue was purified with pre-TLC (DCM:MeOH=90%:10%) to give crude product, which was further purified with pre-HPLC to afford desired product (2.87 mg, 3%). ¹H NMR (400 MHz, DMSO) δ_{H} 11.08 (s, 1H), 7.77 (s, 1H), 7.67 (d, J=8.4 Hz, 1H), 7.49 (d, J=8.0 Hz, 2H), 7.42 (t, J=8.0 Hz, 2H), 7.35 (s, 1H), 7.26 (d, J=8.0 Hz, 1H), 7.17 (t, J=8.0 Hz, 1H), 7.07 (t, J=8.0 Hz, 3H), 6.67 (s, 1H), 5.07 (dd, J=12.0, 4.0 Hz, 1H), 4.42 (d, J=12.0 Hz, 1H), 3.99 (s, 1H), 3.93-3.82 (m, 1H), 3.48 (s, 4H), 3.42-3.35 (m, 5H), 3.29-3.23 (m, 3H), 2.95-2.80 (m, 3H), 2.61-2.54 (m, 7H), 2.30-2.15 (m, 3H), 2.05-1.84 (m, 3H), 1.66 (dd, J=32.0, 12.0 Hz, 2H), 1.50 (s, 5H), 1.18-1.02 (m, 2H); [M+H]⁺=942.8.

### Example 31: (7S)-7-(1-(5-((2-(2, 6-dioxopiperidin-3-yl)-1, 3-dioxoisoindolin-4-yl)amino)pentanoyl)piperidin-4-yl)-2-(4-phenoxyphenyl)-4, 5, 6, 7-tetrahydropyrazolo[1, 5-a]pyrimidine-3-carboxamide

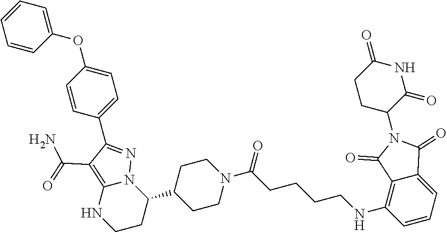 [SMILES: NC(=O)c1c(-c2ccc(Oc3ccccc3)cc2)nn2c1NCC[C@H]2C1CCN(C(=O)CCCCNc2cccc3c2C(=O)N(C2CCC(=O)NC2=O)C3=O)CC1]

The titled compound was synthesized in the procedures similar to Example 23. ¹H NMR (400 MHz, DMSO) δ_{H} 11.10 (s, 1H), 7.57 (t, J=7.8 Hz, 1H), 7.50 (d, J=8.4 Hz, 2H), 7.42 (t, J=7.8 Hz, 2H), 7.17 (t, J=7.3 Hz, 1H), 7.06 (dt, J=16.5, 7.6 Hz, 6H), 6.67 (s, 1H), 6.56 (s, 1H), 5.04 (dd, J=12.8, 5.2 Hz, 1H), 4.44 (d, J=11.7 Hz, 1H), 4.02-3.84 (m, 2H), 3.30 (m, 4H), 2.89 (dd, J=27.3, 13.0 Hz, 2H), 2.63 (d, J=29.4 Hz, 1H), 2.33 (s, 3H), 2.21 (s, 1H), 2.01 (d, J=11.2 Hz, 2H), 1.91 (s, 1H), 1.67 (dd, J=36.1, 14.9 Hz, 1H), 1.57 (s, 5H), 1.32-1.08 (m, 3H); [M+H]⁺=772.9

### Example 32: (7S)-7-(1-(4-((2-(2, 6-dioxopiperidin-3-yl)-1, 3-dioxoisoindolin-4-yl)amino)butanoyl)piperidin-4-yl)-2-(4-phenoxyphenyl)-4, 5, 6, 7-tetrahydropyrazolo[1, 5-a]pyrimidine-3-carboxamide

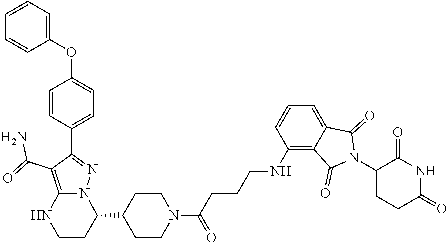 [SMILES: NC(=O)c1c(-c2ccc(Oc3ccccc3)cc2)nn2c1NCC[C@H]2C1CCN(C(=O)CCCNc2cccc3c2C(=O)N(C2CCC(=O)NC2=O)C3=O)CC1]

The titled compound was synthesized in the procedures similar to Example 23. ¹H NMR (400 MHz, DMSO) δ_{H} 11.09 (s, 1H), 7.58 (t, J=6.8 Hz, 1H), 7.50 (d, J=8.4 Hz, 2H), 7.42 (t, J=7.8 Hz, 2H), 7.20-7.14 (m, 2H), 7.11-6.98 (m, 5H), 6.66 (s, 2H), 5.05 (dd, J=12.8, 5.2 Hz, 1H), 4.47 (d, J=11.2 Hz, 1H), 3.95 (dd, J=32.0, 8.8 Hz, 2H), 3.05-2.79 (m, 3H), 2.65-2.54 (m, 2H), 2.46-2.15 (m, 5H), 2.05-1.61 (m, 7H), 1.51 (t, J=12.3 Hz, 1H), 1.32-1.09 (m, 3H); [M+H]⁺=758.8.

### Example 33: (7S)-7-(1-(3-(2-((2-(2, 6-dioxopiperidin-3-yl)-1, 3-dioxoisoindolin-4-yl)amino)-N-methylacetamido)propanoyl)piperidin-4-yl)-2-(4-phenoxyphenyl)-4, 5, 6, 7-tetrahydropyrazolo[1,5-a]pyrimidine-3-carboxamide

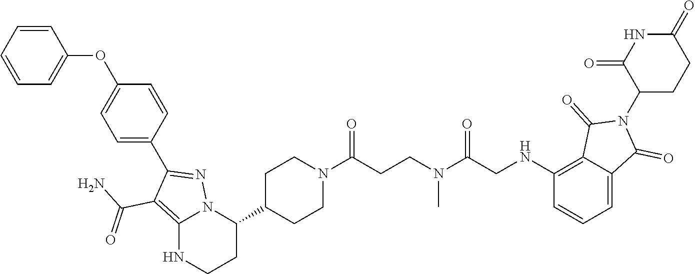 [SMILES: CN(CCC(=O)N1CCC([C@@H]2CCNc3c(C(N)=O)c(-c4ccc(Oc5ccccc5)cc4)nn32)CC1)C(=O)CNc1cccc2c1C(=O)N(C1CCC(=O)NC1=O)C2=O]

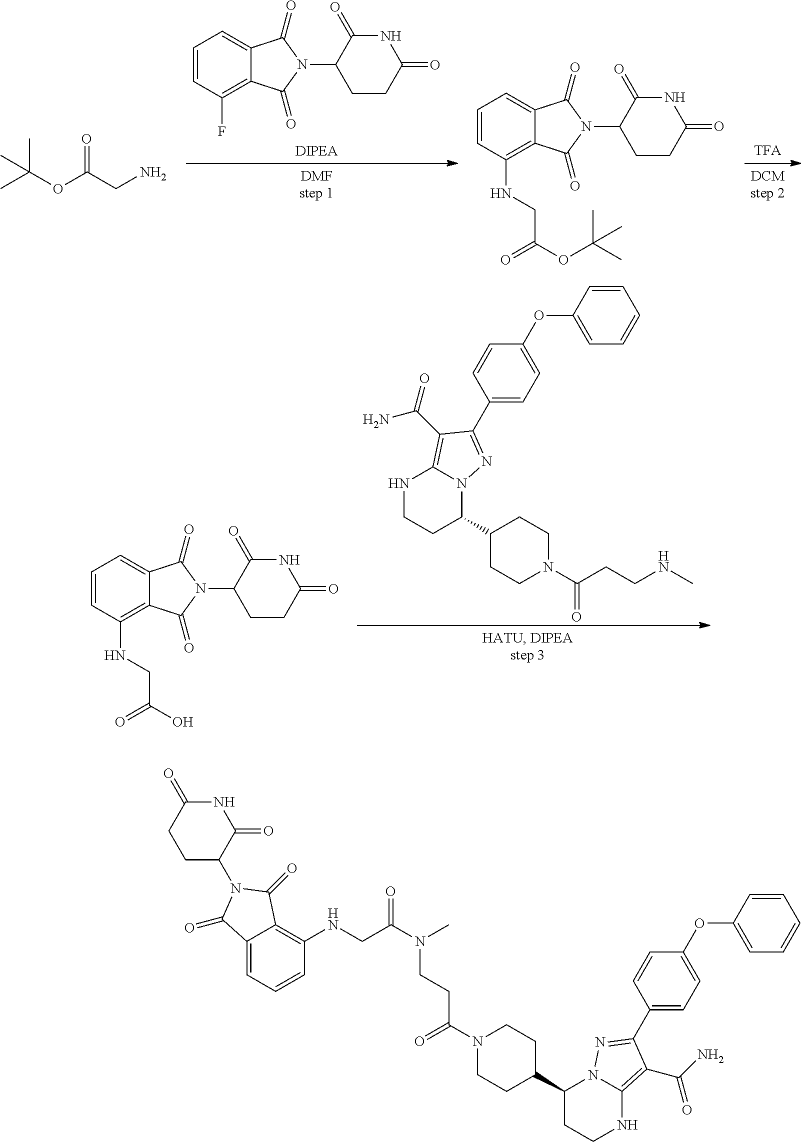

### Step 1: tert-butyl (2-(2, 6-dioxopiperidin-3-yl)-1, 3-dioxoisoindolin-4-yl)glycinate

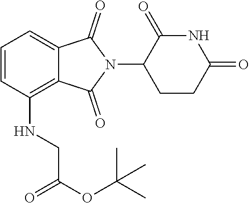 [SMILES: CC(C)(C)OC(=O)CNc1cccc2c1C(=O)N(C1CCC(=O)NC1=O)C2=O]

A mixture of 2-(2, 6-dioxopiperidin-3-yl)-4-fluoroisoindoline-1, 3-dione (0.5 g, 1.8 mmol), tert-butyl glycinate (0.236 g, 1.8 mmol) and DIPEA (0.35 g, 2.7 mmol) in DMF (10 mL) was stirred at 80° C. in a sealed tube for 8 h. LCMS showed the reaction was completed. The solvent was evaporated in vacuum to afford the crude product, which was further purified with silica gel column chromatography (DCM:MeOH=40: 1-15:1 gradient elution) to give the product (40 mg, 5.8%). [M+H]⁺=388.0.

### Step 2: (2-(2, 6-dioxopiperidin-3-yl)-1, 3-dioxoisoindolin-4-yl)glycine

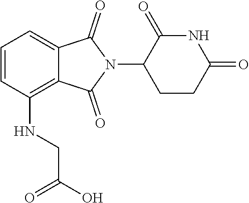 [SMILES: O=C(O)CNc1cccc2c1C(=O)N(C1CCC(=O)NC1=O)C2=O]

A mixture of tert-butyl (2-(2, 6-dioxopiperidin-3-yl)-1, 3-dioxoisoindolin-4-yl)glycinate (40 mg, 0.1 mmol) and TFA (2 mL) in DCM (3 mL) was stirred at R.T. for 2 hour. LCMS showed the reaction was completed. The solvent was removed in vacuo to give the product (38 mg, 99%), which was used in the next step without further purification. [M+H]⁺=332.0.

### Step 3: (7S)-7-(1-(3-(2-((2-(2, 6-dioxopiperidin-3-yl)-1, 3-dioxoisoindolin-4-yl)amino)-N-methylacetamido)propanoyl)piperidin- 4-yl)-2-(4-phenoxyphenyl)-4, 5, 6, 7-tetrahydropyrazolo[1, 5-a]pyrimidine-3-carboxamide

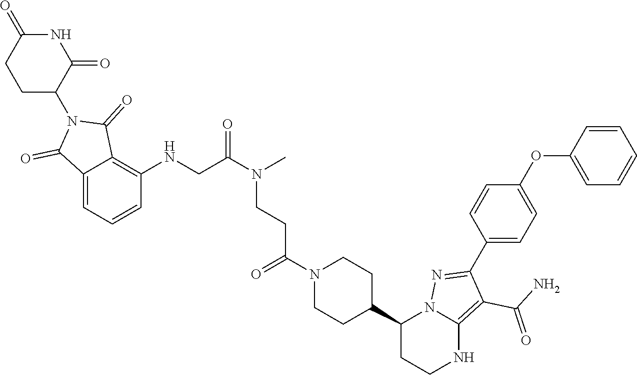 [SMILES: CN(CCC(=O)N1CCC([C@@H]2CCNc3c(C(N)=O)c(-c4ccc(Oc5ccccc5)cc4)nn32)CC1)C(=O)CNc1cccc2c1C(=O)N(C1CCC(=O)NC1=O)C2=O]

A mixture of (2-(2, 6-dioxopiperidin-3-yl)-1, 3-dioxoisoindolin-4-yl)glycine (32 mg, 0.0956 mmol), (S)-7-(1-(3-(methylamino)propanoyl)piperidin-4-yl)-2-(4-phenoxyphenyl)-4, 5, 6, 7-tetrahydropyrazolo[1,5-a]pyrimidine-3-carboxamide (40 mg, 0.0797 mmol), HATU (60.6 g, 0.1594 mmol) and DIPEA (31 mg, 0.239 mmol) in DMF (10 mL) was stirred at room temperature overnight. LCMS showed the reaction was completed. The solvent was evaporated in vacuum. The crude product thus obtained was further purified with pre-TLC (DCM:MeOH=10:1) to give the product (15 mg, 19.5%). ¹H NMR (400 MHz, DMSO) δ_{H} 11.11 (s, 1H), 7.60 (dd, J=15.2, 7.6 Hz, 1H), 7.50 (d, J=8.0 Hz, 2H), 7.41 (t, J=7.6 Hz, 2H), 7.17 (t, J=7.2 Hz, 1H), 7.07 (t, J=10.4 Hz, 7H), 6.67 (s, 1H), 5.07 (dd, J=12.4, 4.8 Hz, 1H), 4.46 (s, 1H), 4.30-3.82 (m, 4H), 3.55 (d, J=6.0 Hz, 2H), 2.98-2.90 (m, 2H), 2.70-2.55 (m, 3H), 2.30-2.15 (m, 2H), 2.12-1.85 (m, 4H), 1.75-1.45 (m, 3H), 1.40-1.10 (m, 4H); [M+H]⁺=816.0.

### Example 34: (7S)-7-(1-(1-((2-(2, 6-dioxopiperidin-3-yl)-1, 3-dioxoisoindolin-4-yl)amino)-14-oxo-3, 6, 9, 12-tetraoxa-15-azanonadecan-19-oyl)piperidin-4-yl)-2-(4-phenoxyphenyl)-4, 5, 6, 7-tetrahydropyrazolo[1, 5-a]pyrimidine-3-carboxamide

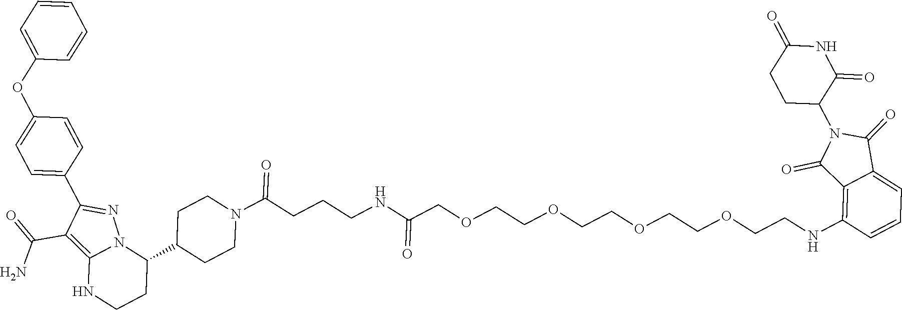 [SMILES: NC(=O)c1c(-c2ccc(Oc3ccccc3)cc2)nn2c1NCC[C@H]2C1CCN(C(=O)CCCNC(=O)COCCOCCOCCOCCNc2cccc3c2C(=O)N(C2CCC(=O)NC2=O)C3=O)CC1]

The titled compound was synthesized in the procedures similar to Example 23. ¹H NMR (400 MHz, DMSO) δ_{H} 11.10 (s, 1H), 7.70 (s, 1H), 7.61-7.54 (m, 1H), 7.50 (d, J=8.5 Hz, 2H), 7.41 (t, J=7.8 Hz, 2H), 7.16 (dd, J=16.5, 8.1 Hz, 2H), 7.06 (dd, J=17.1, 8.1 Hz, 5H), 6.67 (s, 1H), 6.60 (s, 1H), 5.05 (dd, J=12.8, 5.4 Hz, 1H), 4.42 (s, 1H), 4.00 (s, 1H), 3.84 (s, 3H), 3.61 (t, J=5.3 Hz, 2H), 3.52 (d, J=10.1 Hz, 12H), 3.46 (d, J=5.5 Hz, 2H), 3.30-3.26 (m, 1H), 3.10 (d, J=6.8 Hz, 2H), 2.89 (dd, J=23.1, 11.7 Hz, 2H), 2.58 (d, J=16.3 Hz, 2H), 2.43 (s, 1H), 2.27 (s, 3H), 2.06-1.83 (m, 3H), 1.74-1.58 (m, 3H), 1.51 (s, 1H), 1.25-1.20 (m, 3H); [M+H]⁺=991.9.

### Example 35: (7S)-7-(1-(2-(2-(2-(5-(4-(2-(2, 6-dioxopiperidin-3-yl)-1, 3-dioxoisoindolin-5-yl)piperazin-1-yl)pentanamido)ethoxy)ethoxy)acetyl)piperidin-4-yl)-2-(4-phenoxyphenyl)-4, 5, 6, 7-tetrahydropyrazolo[1,5-a]pyrimidine-3-carboxamide

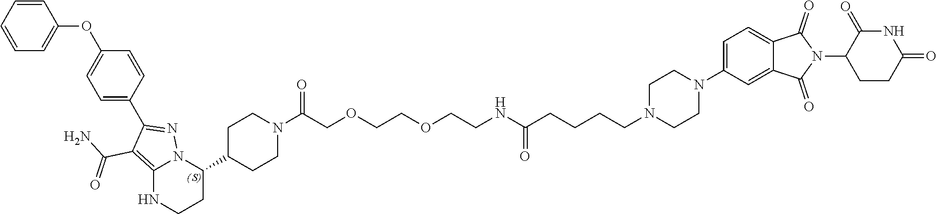

The titled compound was synthesized in the procedures similar to Example 23. ¹H NMR (400 MHz, DMSO) δ_{H} 11.08 (s, 1H), 7.84 (s, 1H), 7.67 (d, J=8.0 Hz, 1H), 7.49 (d, J=8.0 Hz, 2H), 7.41 (t, J=8.0 Hz, 2H), 7.33 (s, 1H), 7.24 (d, J=8.0 Hz, 1H), 7.17 (t, J=8.0 Hz, 1H), 7.06 (t, J=8.0 Hz, 4H), 6.67 (s, 1H), 5.07 (d, J=8.0 Hz, 1H), 4.38 (d, J=12.0 Hz, 1H), 4.12 (dd, J=32.0, 12.0 Hz, 2H), 4.00 (s, 1H), 3.81 (s, 1H), 3.51 (s, 5H), 3.40 (s, 7H), 3.18 (s, 2H), 2.97-2.80 (m, 3H), 2.70-2.58 (m, 3H), 2.33-2.14 (m, 4H), 2.14-1.84 (m, 6H), 1.68 (s, 1H), 1.58-1.37 (m, 5H), 1.32-1.07 (m, 3H); [M+H]⁺=986.9.

### Example 36: (7S)-7-(1-(4-(4-(2-((2-(2, 6-dioxopiperidin-3-yl)-1, 3-dioxoisoindolin-4-yl)oxy)acetamido)butanamido)butanoyl)piperidin-4-yl)-2-(4-phenoxyphenyl)-4, 5, 6, 7-tetrahydropyrazolo[1,5-a]pyrimidine-3-carboxamide

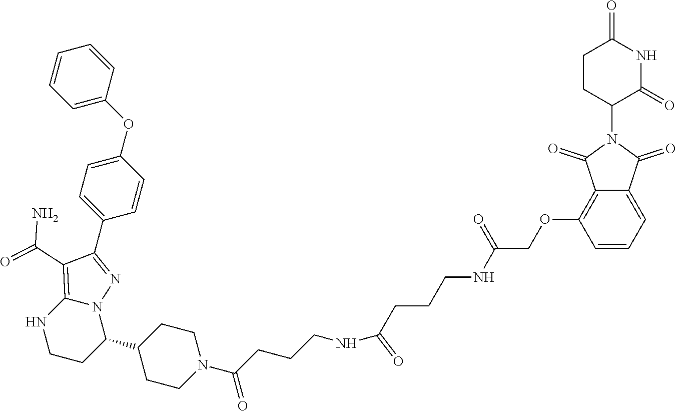 [SMILES: NC(=O)c1c(-c2ccc(Oc3ccccc3)cc2)nn2c1NCC[C@H]2C1CCN(C(=O)CCCNC(=O)CCCNC(=O)COc2cccc3c2C(=O)N(C2CCC(=O)NC2=O)C3=O)CC1]

The titled compound was synthesized in the procedure similar to Example 23. ¹H NMR (400 MHz, DMSO) δ_{H} 11.12 (s, 1H), 7.99 (s, 1H), 7.84-7.75 (m, 2H), 7.54-7.45 (m, 3H), 7.40 (dd, J=16.5, 8.3 Hz, 3H), 7.17 (t, J=7.2 Hz, 1H), 7.07 (t, J=9.0 Hz, 4H), 6.67 (s, 1H), 5.12 (dd, J=12.8, 5.1 Hz, 1H), 4.76 (s, 2H), 4.44 (d, J=12.0 Hz, 1H), 3.93 (d, J=52.4 Hz, 2H), 3.12 (d, J=6.1 Hz, 2H), 3.02 (d, J=5.7 Hz, 2H), 2.92 (d, J=13.9 Hz, 3H), 2.59 (d, J=16.2 Hz, 2H), 2.30 (d, J=20.7 Hz, 5H), 2.05 (dd, J=17.1, 9.7 Hz, 4H), 1.91 (s, 1H), 1.73-1.47 (m, 6H), 1.25-1.20 (m, 3H); [M+H]⁺=901.8.

### Example 37: (7S)-7-(1-(3-((2-(2-((2-(2, 6-dioxopiperidin-3-yl)-1, 3-dioxoisoindolin-4-yl)amino)ethoxy)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

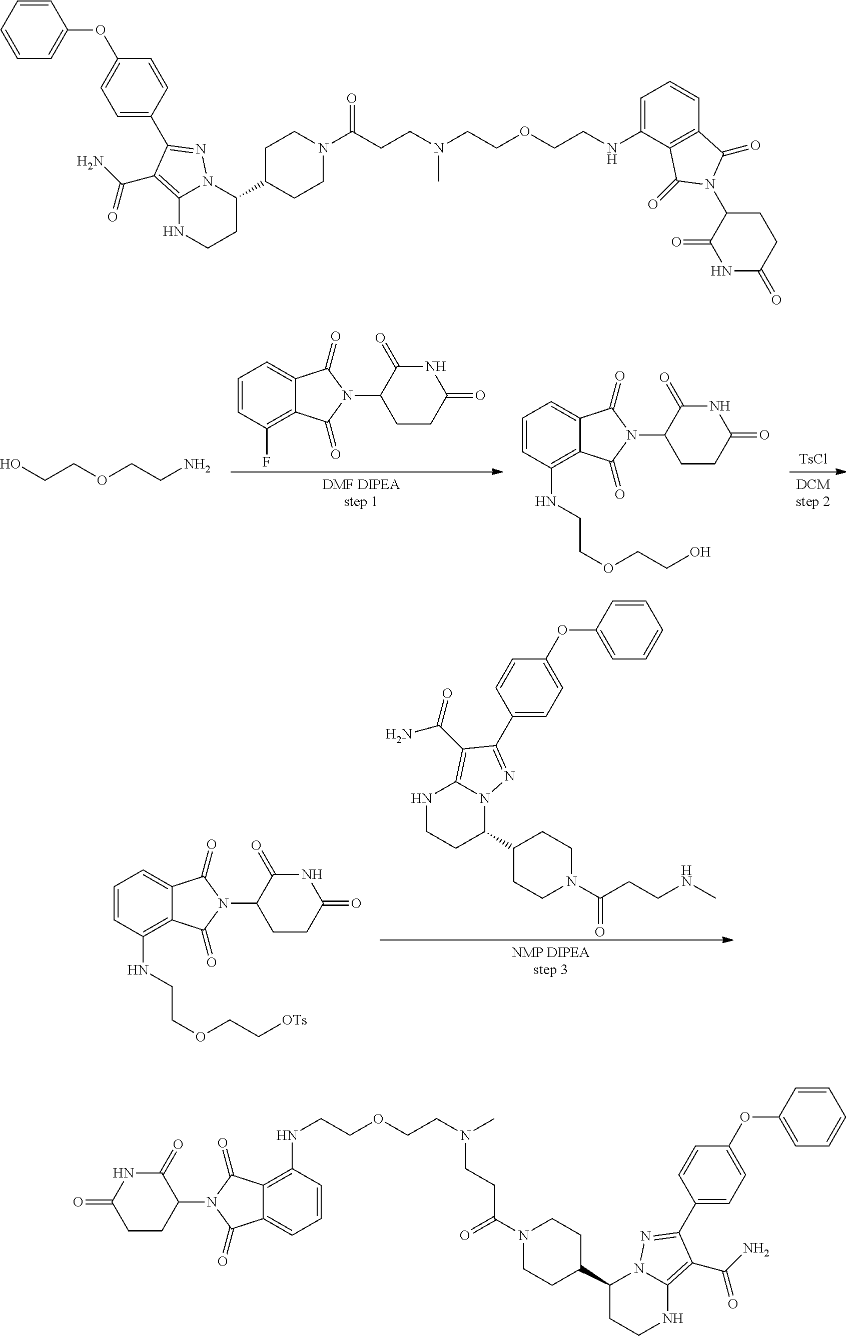

### Step 1: 2-(2, 6-dioxopiperidin-3-yl)-4-((2-(2-hydroxyethoxy)ethyl)amino)isoindoline-1, 3-dione

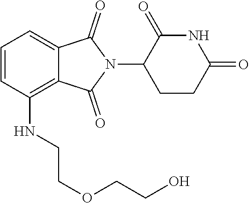 [SMILES: O=C1CCC(N2C(=O)c3cccc(NCCOCCO)c3C2=O)C(=O)N1]

A mixture of 2-(2, 6-dioxopiperidin-3-yl)-4-fluoroisoindoline-1, 3-dione (0.5 g, 1.8 mmol), 2-(2-aminoethoxy)ethan-1-ol (0.227 g, 2.16 mmol) and DIPEA (0.7 g, 5.4 mmol) in DMF (10 mL) was stirred at 80° C. in a sealed tube for 8 h. LCMS showed the reaction was completed. The solvent was evaporated in vacuum to afford the crude product, which was further purified with silica gel column chromatography (DCM:MeOH=40: 1-15:1 gradient elution) to give the product (0.2 g, 31%). [M+H]⁺=362.0.

### Step 2: 2-(2-((2-(2, 6-dioxopiperidin-3-yl)-1, 3-dioxoisoindolin-4-yl)amino)ethoxy) ethyl 4-methylbenzenesulfonate

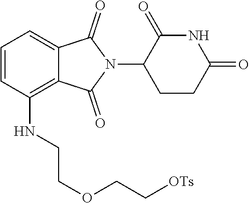 [SMILES: Cc1ccc(S(=O)(=O)OCCOCCNc2cccc3c2C(=O)N(C2CCC(=O)NC2=O)C3=O)cc1]

To a solution of 2-(2, 6-dioxopiperidin-3-yl)-4-((2-(2-hydroxyethoxy)ethyl)amino) isoindoline-1, 3-dione (0.2 g, 0.554 mmol) and Et₃N (112 mg, 1.108 mmol) in DCM (10 mL) was added drop-wise TsCl (105.6 mg, 0.554 mmol) at 0° C. The mixture was stirred for 3 hours at room temperature. TLC (DCM:MeOH=20:1, Rf=0.4) showed the reaction was completed. Then was added aqueous NH₄Cl and extracted with DCM (50 mL*3). The combined organic layer was dried over Na₂SO₄. Filtered and concentrated to give the crude product, which was purified with silica gel chromatography (DCM:MeOH=100:1-20 :1) to give the product (0.25 g, 89%). [M+H]⁺=516.0.

### Step 3: (7S)-7-(1-(3-((2-(2-((2-(2, 6-dioxopiperidin-3-yl)-1, 3-dioxoisoindolin-4-yl)amino) ethoxy)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

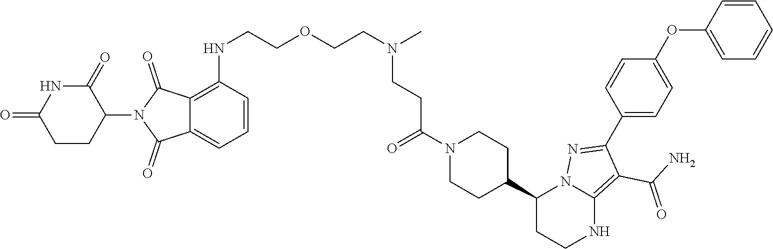 [SMILES: CN(CCOCCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

A mixture of 2-(2-((2-(2, 6-dioxopiperidin-3-yl)-1, 3-dioxoisoindolin-4-yl)amino) ethoxy) ethyl 4-methylbenzenesulfonate (0.1 g, 0.199 mmol), (S)-7-(1-(3-(methylamino)propanoyl)piperidin-4-yl)-2-(4-phenoxyphenyl)-4, 5, 6, 7-tetrahydropyrazolo[1, 5-a]pyrimidine-3-carboxamide (102 mg, 0.199 mmol) and DIPEA (55 mg, 0.398 mmol) in NMP (5 mL) was stirred at 110° C. in a sealed tube for 8 h. LCMS showed the reaction was completed. The solvent was evaporated in vacuum to afford the crude product, which was further purified with C18 column chromatography (water: MeCN=40: 1-5:1 gradient elution) to give the product (40 mg, 23.8%). ¹H NMR (400 MHz, DMSO) δ_{H} 11.09 (s, 1H), 7.60-7.35 (m, 6H), 7.23-6.99 (m, 8H), 6.70-6.54 (m, 2H), 5.10-5.50 (m, 1H), 4.41 (s, 1H), 4.04-3.80 (m, 4H), 3.65-3.41 (m, 11H), 3.02-2.80 (m, 7H), 2.70-2.55 (m, 7H), 2.45-2.30 (m, 4H), 2.19 (s, 3H), 2.10-1.85 (m, 6H), 1.73-1.45 (m, 9H); [M+H]⁺=846.0.

### Example 38: (7S)-7-(1-(3-((2-((2-(2, 6-dioxopiperidin-3-yl)-1, 3-dioxoisoindolin-4-yl)amino)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

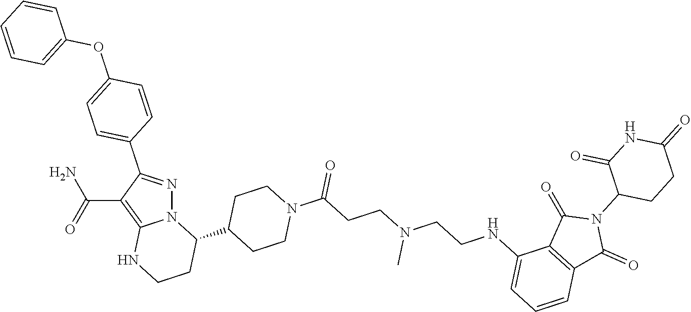 [SMILES: CN(CCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

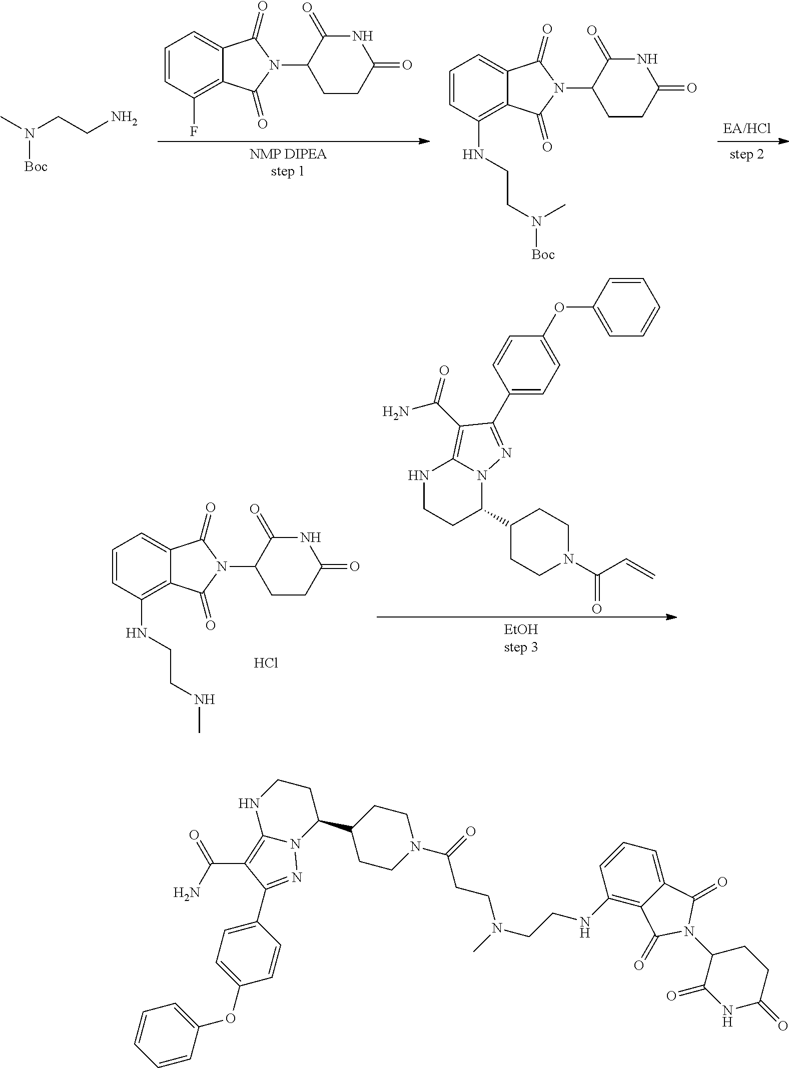

### Step 1: tert-butyl (2-((2-(2, 6-dioxopiperidin-3-yl)-1, 3-dioxoisoindolin-4-yl)amino)ethyl)(methyl)carbamate

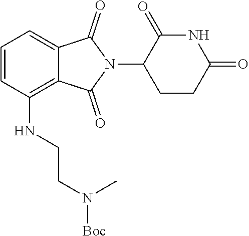 [SMILES: CN(CCNc1cccc2c1C(=O)N(C1CCC(=O)NC1=O)C2=O)C(=O)OC(C)(C)C]

A mixture of 2-(2, 6-dioxopiperidin-3-yl)-4-fluoroisoindoline-1, 3-dione (0.5 g, 1.8 mmol), tert-butyl (2-aminoethyl)(methyl)carbamate (376 mg, 2.16 mmol) and DIPEA (0.7 g, 5.4 mmol) in NMP (5 mL) was stirred at 110° C. in a sealed tube for 8 h. LCMS showed the reaction was completed. Saturated aqueous NaCl was added to quench the reaction and the reaction mixture was extracted with DCM. The organic layer was dried over anhydrous Na₂SO₄ and evaporated in vacuum to afford the crude product, which was further purified with pre-TLC (DCM:MeOH=50:1) to give the product (0.7 g, 87.5%). [M+H]⁺=431.0.

### Step 2: 2-(2, 6-dioxopiperidin-3-yl)-4-((2-(methylamino)ethyl)amino)isoindoline-1, 3-dione hydrochloride

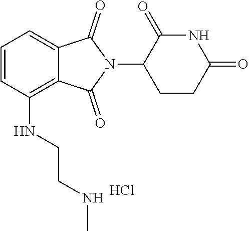 [SMILES: CNCCNc1cccc2c1C(=O)N(C1CCC(=O)NC1=O)C2=O ; Cl]

A mixture of tert-butyl (2-((2-(2, 6-dioxopiperidin-3-yl)-1, 3-dioxoisoindolin-4-yl)amino)ethyl)(methyl)carbamate (0.7 mg, 1.62 mmol) in EA (5 mL) was added drop-wise EA/HCl (10 mL) and was stirred at R. T. for 2 hour. LCMS showed the reaction was completed. The solvent was removed in vacuo to give the product (0.55 mg, 90%), which was used for the next step without further purification. [M+H]⁺=331.0.

### Step 3: (7S)-7-(1-(3-((2-((2-(2, 6-dioxopiperidin-3-yl)-1, 3-dioxoisoindolin-4-yl)amino)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

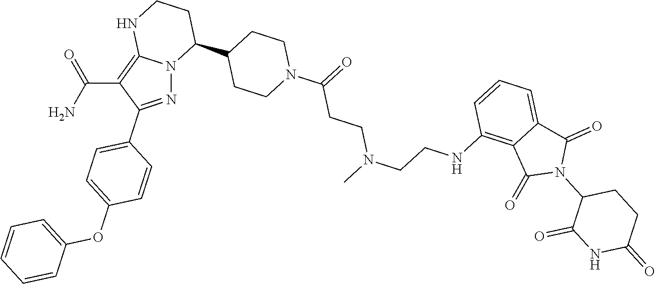 [SMILES: CN(CCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

A mixture of (S)-7-(1-acryloylpiperidin-4-yl)-2-(4-phenoxyphenyl)-4, 5, 6, 7-tetrahydropyrazolo[1,5-a]pyrimidine-3-carboxamide (0.1 g, 0.212 mmol) and 2-(2, 6-dioxopiperidin-3-yl)-4-((2-(methylamino)ethyl)amino)isoindoline-1, 3-dione (70 mg, 0.212 mmol) in EtOH (20 mL) was stirred at 80° C. in a sealed tube for 8h. LCMS showed the reaction was completed. The solvent was evaporated under reduced pressure. The residue was further purified with pre-TLC (DCM:MeOH=50:1) to give the title product (30 mg, 17.7%). ¹H NMR (400 MHz, DMSO) δ_{H} 11.09 (s, 1H), 7.58 (t, J=8.0 Hz, 1H), 7.49 (d, J=8.0 Hz, 2H), 7.42 (t, J=8.0 Hz, 2H), 7.17 (t, J=8.0 Hz, 1H), 7.13-6.99 (m, 7H), 6.68 (d, J=12.0 Hz, 2H), 5.04 (dd, J=12, 4.0 Hz, 1H), 4.43 (d, J=12.0 Hz, 1H), 4.05-3.83 (m, 3H), 3.34-3.26 (m, 6H), 3.04-2.78 (m, 4H), 2.70-2.56 (m, 7H), 2.23 (s, 3H), 2.05-1.84 (m, 4H), 1.75-1.61 (m, 2H), 1.56-1.40 (m, 3H); [M+H]⁺=802.0.

### Example 39: (7S)-7-(1-(4-((6-((2-(2, 6-dioxopiperidin-3-yl)-1, 3-dioxoisoindolin-4-yl)amino)hexyl)(methyl)amino)butanoyl)piperidin-4-yl)-2-(4-phenoxyphenyl)-4, 5, 6, 7-tetrahydropyrazolo[1,5-a]pyrimidine-3-carboxamide

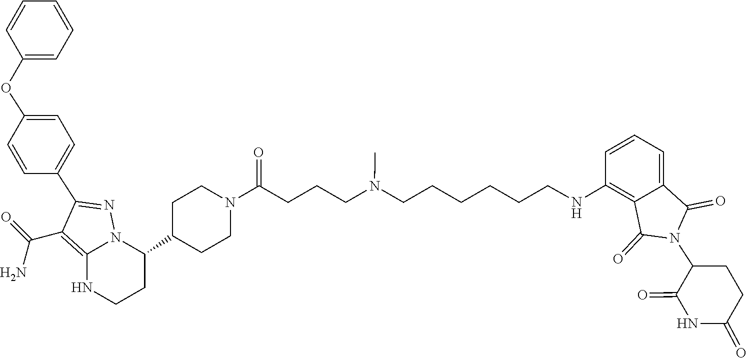 [SMILES: CN(CCCCCCNc1cccc2c1C(=O)N(C1CCC(=O)NC1=O)C2=O)CCCC(=O)N1CCC([C@@H]2CCNc3c(C(N)=O)c(-c4ccc(Oc5ccccc5)cc4)nn32)CC1]

### Example 40: (7S)-7-(1-(3-(4-(4-(2-(2, 6-dioxopiperidin-3-yl)-1, 3-dioxoisoindolin-5-yl)piperazin-1-yl)phenyl)propanoyl)piperidin-4-yl)-2-(4-phenoxyphenyl)-4, 5, 6, 7-tetrahydropyrazolo[1,5-a]pyrimidine-3-carboxamide

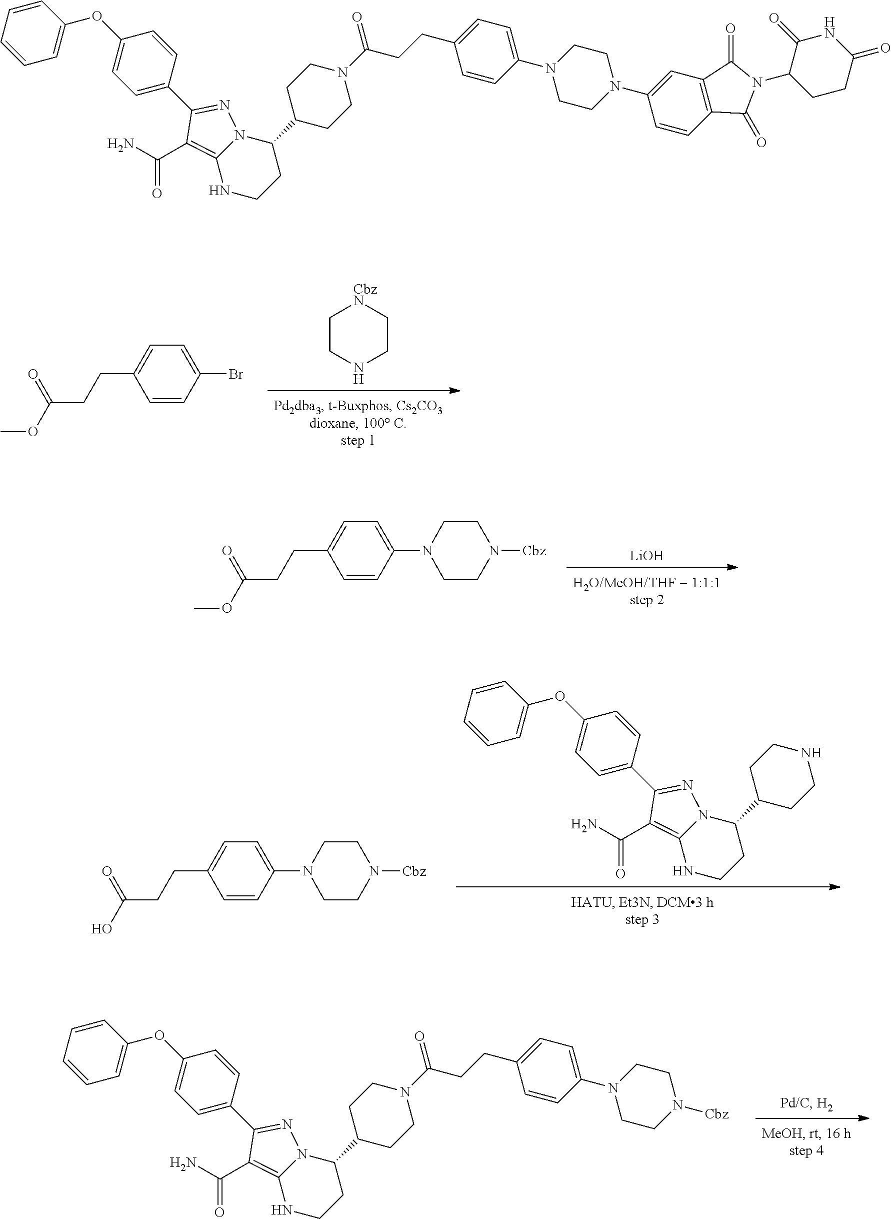

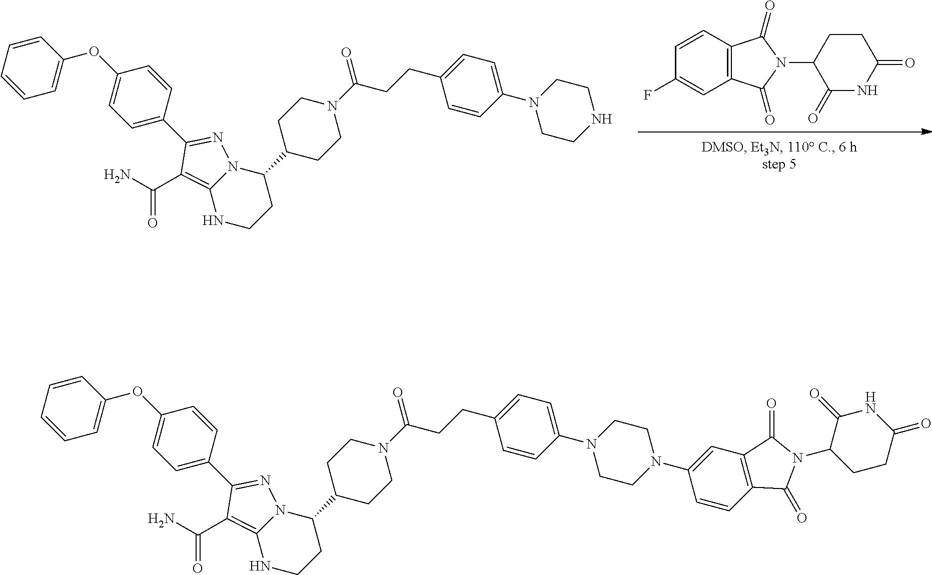 [SMILES: NC(=O)c1c(-c2ccc(Oc3ccccc3)cc2)nn2c1NCC[C@H]2C1CCN(C(=O)CCc2ccc(N3CCNCC3)cc2)CC1 ; O=C1CCC(N2C(=O)c3ccc(F)cc3C2=O)C(=O)N1 ; NC(=O)c1c(-c2ccc(Oc3ccccc3)cc2)nn2c1NCC[C@H]2C1CCN(C(=O)CCc2ccc(N3CCN(c4ccc5c(c4)C(=O)N(C4CCC(=O)NC4=O)C5=O)CC3)cc2)CC1]

### Step 1: Benzyl 4-(4-(3-methoxy-3-oxopropyl)phenyl)piperazine-1-carboxylate

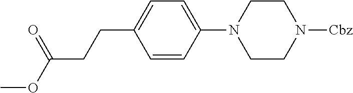 [SMILES: COC(=O)CCc1ccc(N2CCN(C(=O)OCc3ccccc3)CC2)cc1]

To a solution of methyl 3-(4-bromophenyl)propanoate (1.21 g, 5 mmol) and benzyl piperazine-1-carboxylate (1.10 g, 5 mmol) dissolved in dioxane (50 ml) were added Cs₂CO₃ (3.26 g, 10 mmol), t-Buxphos (300 mg, 0.7 mmol) and Pd₂dba₃ (150 mg, 0.16 mmol). The mixture was stirred at 100° C. overnight and cooled down to R.T. The solution was washed with water 100 mL and extracted with EA (150 mL×3). The organic layer was combined, dried over Na₂SO₄, filtered and concentrated to dryness. The residue was purified with silica gel column chromatography (PE:EA=9/1 to 2/3) to afford benzyl 4-(4-(3-methoxy-3-oxopropyl) phenyl)piperazine-1-carboxylate (780 mg, 41%). [M+H]⁺=382.9.

### Step 2: 3-(4-(4-((benzyloxy)carbonyl)piperazin-1-yl)phenyl)propanoic Acid

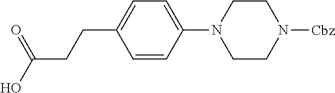 [SMILES: O=C(O)CCc1ccc(N2CCN(C(=O)OCc3ccccc3)CC2)cc1]

To a mixture of benzyl 4-(4-(3-methoxy-3-oxopropyl)phenyl)piperazine-1-carboxylate (780 mg, 2.04 mmol) dissolved in THF (3 mL)/MeOH (3 mL)/H₂O (3 mL) were added LiOH (245 mg, 10.2 mmol). The mixture was stirred at R.T. overnight. Solvent was removed under reduced pressure. The pH value of the mixture was adjusted to 3-5. The mixture was extracted with EA (20 mL×3). The organic layers were combined, dried over Na₂SO₄ and concentrated to obtain 3-(4-(4-((benzyloxy)carbonyl)piperazin-1-yl)phenyl)propanoic acid (650 mg, 87%). [M+H]⁺=368.9.

### Step 3: Benzyl (S)-4-(4-(3-(4-(3-carbamoyl-2-(4-phenoxyphenyl)-4, 5, 6, 7-tetrahydropyrazolo[1, 5-a]pyrimidin-7-yl)piperidin-1-yl)-3-oxopropyl)phenyl)piperazine-1-carboxylate

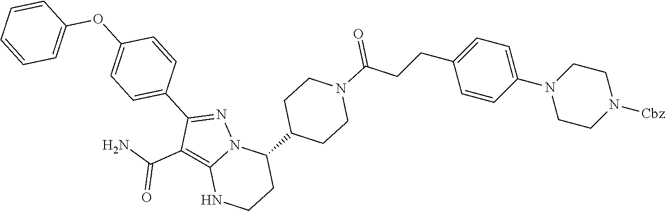 [SMILES: NC(=O)c1c(-c2ccc(Oc3ccccc3)cc2)nn2c1NCC[C@H]2C1CCN(C(=O)CCc2ccc(N3CCN(C(=O)OCc4ccccc4)CC3)cc2)CC1]

To a solution of 3-(4-(4-((benzyloxy)carbonyl)piperazin-1-yl)phenyl)propanoic acid (184.1 mg, 0.5 mmol, 1.00 eq) in DCM (15 mL) was added HATU (228 mg, 0.6 mmol). The mixture was stirred at R.T. for 10 min. Then, Et₃N (152 mg, 1.50 mmol) and (S)-2-(4-phenoxyphenyl)-7-(piperidin-4-yl)-4, 5, 6, 7-tetrahydropyrazolo[1,5-a]pyrimidine-3-carboxamide (209 mg, 0.5 mmol) were added to the mixture. The reaction was stirred at R.T. for 2 h. The mixture was washed with water (20 mL) and extracted with DCM (30 mL×3). The organic layers were combined and dried over Na₂SO₄. The mixture was filtered and filtrate was concentrated in vacuum. The residue was purified with silica gel column chromatography (DCM:MeOH=100/1 to 9/1) to afford benzyl (5)-4-(4-(3-(4-(3-carbamoyl-2-(4-phenoxyphenyl)-4, 5, 6, 7-tetrahydropyrazolo[1,5-a]pyrimidin-7-yl)piperidin-1-yl)-3-oxopropyl)phenyl)piperazine-1-carboxylate (200 mg, 52%). [M+H]+=768.0

### Step 4: (S)-2-(4-phenoxyphenyl)-7-(1-(3-(4-(piperazin-1-yl)phenyl)propanoyl)piperidin-4-yl)-4, 5, 6, 7-tetrahydropyrazolo[1,5-a]pyrimidine-3-carboxamide

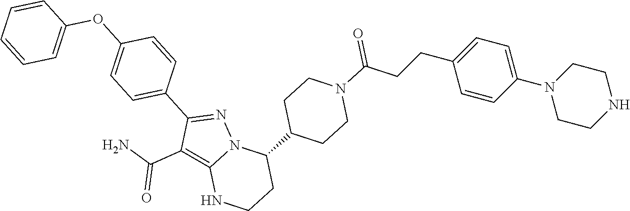 [SMILES: NC(=O)c1c(-c2ccc(Oc3ccccc3)cc2)nn2c1NCC[C@H]2C1CCN(C(=O)CCc2ccc(N3CCNCC3)cc2)CC1]

To a mixture of benzyl (S)-4-(4-(3-(4-(3-carbamoyl-2-(4-phenoxyphenyl)-4, 5, 6, 7-tetrahydropyrazolo[1,5-a]pyrimidin-7-yl)piperidin-1-yl)-3-oxopropyl)phenyl)piperazine-1-carboxylate (200 mg, 0.26 mmol) dissolved in MeOH (10 ml) was added Pd/C (30 mg, w.t. 10%). And the mixture was stirred at room temperature under one hydrogen balloon overnight. Then the mixture was filtered and concentrated to afford (S)-2-(4-phenoxyphenyl)-7-(1-(3-(4-(piperazin-1-yl)phenyl)propanoyl)piperidin-4-yl)-4, 5, 6, 7-tetrahydropyrazolo[1,5-a]pyrimidine-3-carboxamide (120 mg, 56%). [M+H]⁺=634.0.

### Step 5: (7S)-7-(1-(3-(4-(4-(2-(2, 6-dioxopiperidin-3-yl)-1, 3-dioxoisoindolin-5-yl)piperazin-1-yl)phenyl)propanoyl)piperidin-4-yl)-2-(4-phenoxyphenyl)-4, 5, 6, 7-tetrahydropyrazolo[1, 5-a]pyrimidine-3-carboxamide

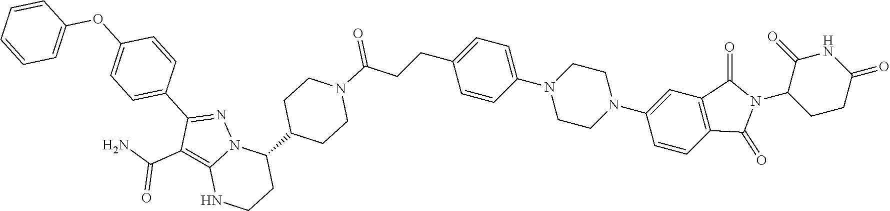 [SMILES: NC(=O)c1c(-c2ccc(Oc3ccccc3)cc2)nn2c1NCC[C@H]2C1CCN(C(=O)CCc2ccc(N3CCN(c4ccc5c(c4)C(=O)N(C4CCC(=O)NC4=O)C5=O)CC3)cc2)CC1]

To a mixture of (S)-2-(4-phenoxyphenyl)-7-(1-(3-(4-(piperazin-1-yl)phenyl) propanoyl)piperidin-4-yl)-4, 5, 6, 7-tetrahydropyrazolo[1,5-a]pyrimidine-3-carboxamide (40 mg, 0.06 mmol) and 2-(2, 6-dioxopiperidin-3-yl)-5-fluoroisoindoline-1, 3-dione (17.4 mg, 0.06 mmol) dissolved in DMSO (5 mL) was added Et₃N (19 mg, 0.19 mmol). The mixture was stirred at room 110° C. for 6 hours. Then, the mixture was cooled to rt and washed with water (15 mL). The mixture was extracted with DCM (10 mL×3). The organic layers were combined and washed with saturated NaCl aq. The organic layers were dried over Na₂SO₄ and filtered. The filtrate was concentrated in vacuum. The residue was purified with pre-TLC (DCM:MeOH=10:1) to afford the title product(8 mg, 14% yield). ¹H NMR (400 MHz, DMSO) δ_{H} 10.82 (s, 1H), 7.68 (d, J=8.5 Hz, 1H), 7.50 (d, J=8.2 Hz, 2H), 7.41-7.35 (m, 3H), 7.28 (d, J=8.7 Hz, 1H), 7.15 (t, J=7.3 Hz, 1H), 7.12-7.02 (m, 6H), 6.88 (d, J=8.2 Hz, 2H), 6.56 (s, 1H), 5.87 (s, 2H), 5.03 (dd, J=12.5, 5.4 Hz, 1H), 3.98 (d, J=5.6 Hz, 1H), 3.59 (s, 4H), 3.28 (d, J=21.2 Hz, 6H), 2.90-2.82 (m, 3H), 2.74 (t, J=7.1 Hz, 2H), 2.62-2.52 (m, 5H), 2.21 (s, 1H), 2.03-1.92 (m, 4H), 1.68 (d, J=12.0 Hz, 1H), 1.49 (s, 1H), 1.25-1.15 (m, 4H); [M+H]⁺=890.0.

### Example 41: (7S)-7-(1-(2-(2-(2-(2-(4-(2-(2, 6-dioxopiperidin-3-yl)-1, 3-dioxoisoindolin-5-yl)piperazin-1-yl)acetamido)ethoxy)ethoxy)acetyl)piperidin-4-yl)-2-(4-phenoxyphenyl)-4, 5, 6, 7-tetrahydropyrazolo[1,5-a]pyrimidine-3-carboxamide

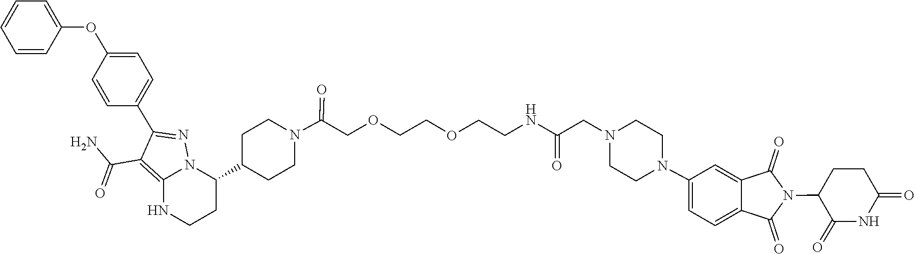 [SMILES: NC(=O)c1c(-c2ccc(Oc3ccccc3)cc2)nn2c1NCC[C@H]2C1CCN(C(=O)COCCOCCNC(=O)CN2CCN(c3ccc4c(c3)C(=O)N(C3CCC(=O)NC3=O)C4=O)CC2)CC1]

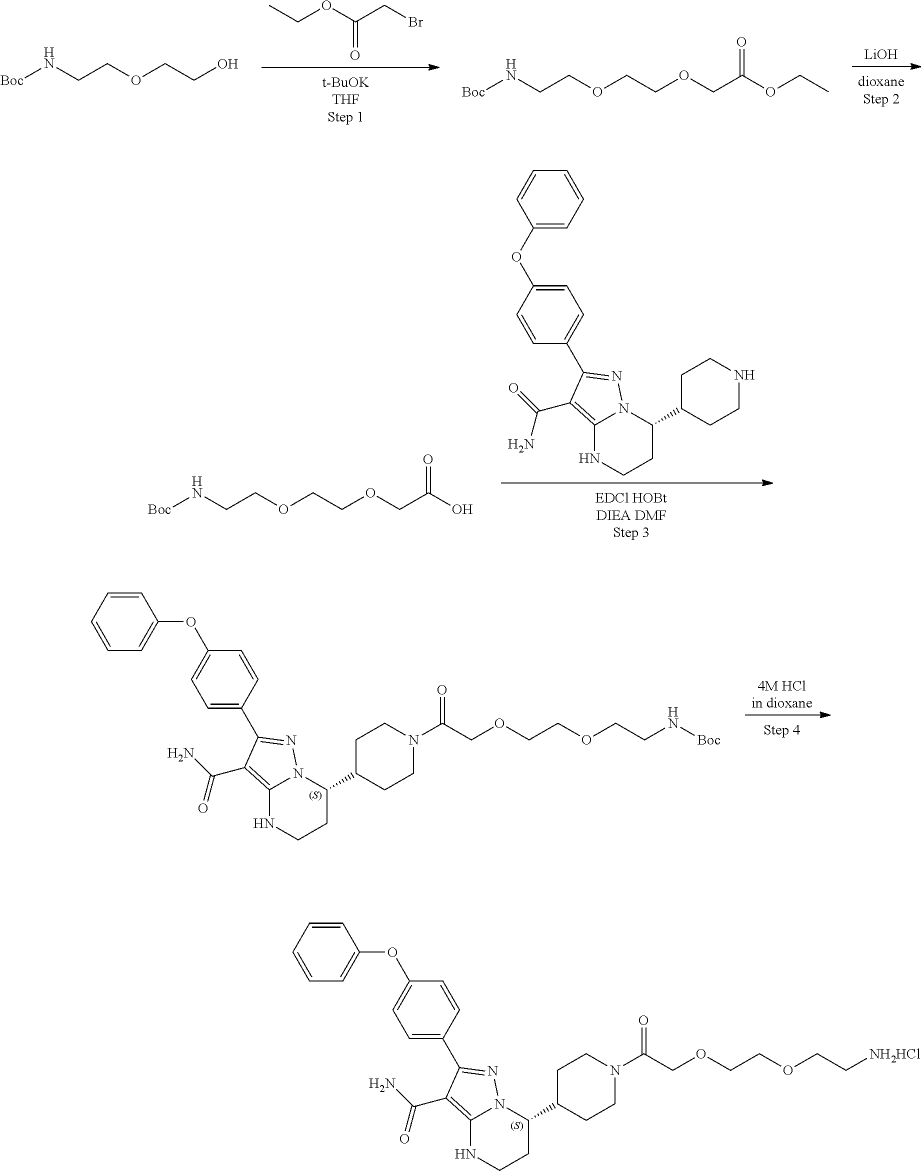

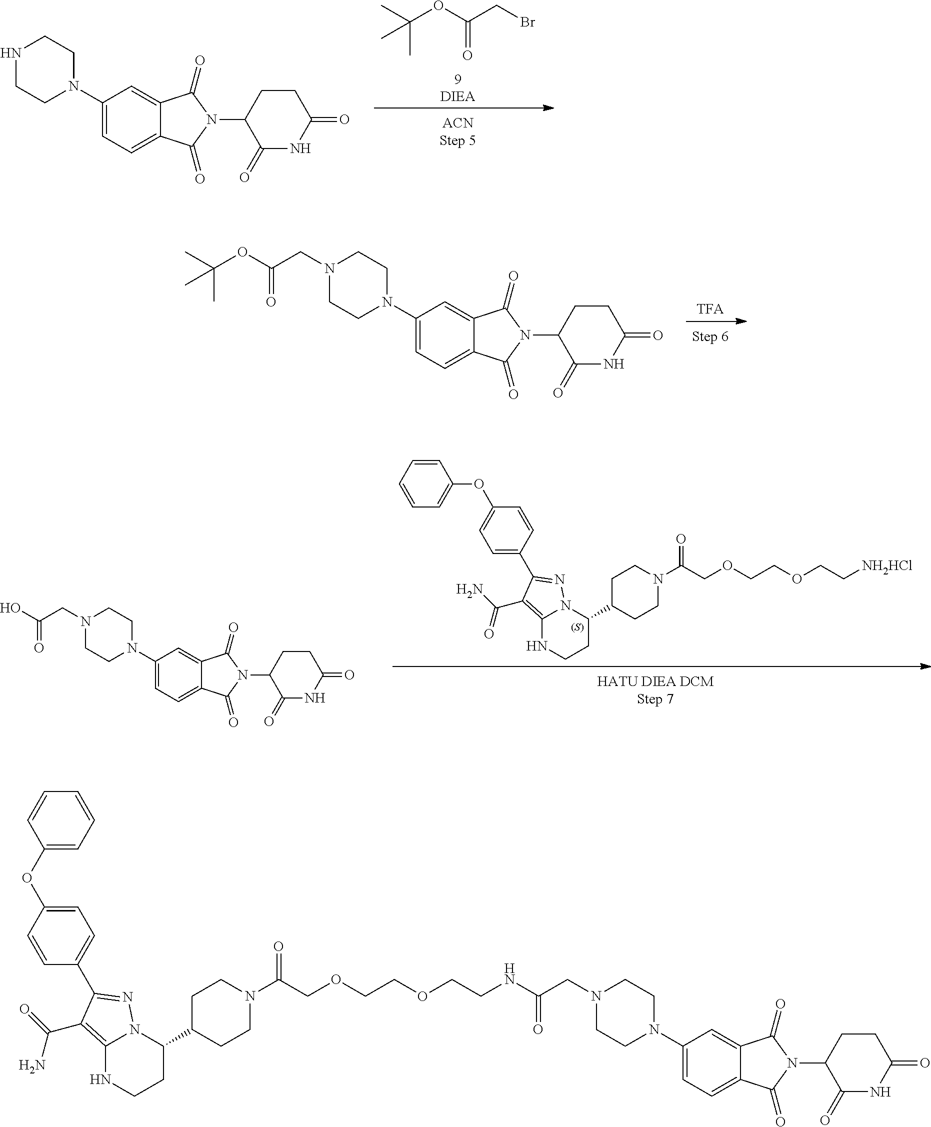

### Step 1: ethyl 2, 2-dimethyl-4-oxo-3, 8, 11-trioxa-5-azatridecan-13-oate

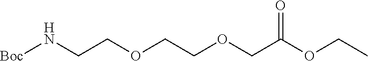 [SMILES: CCOC(=O)COCCOCCNC(=O)OC(C)(C)C]

The tert-butyl (2-(2-hydroxyethoxy)ethyl)carbamate (16.34 g, 75.581 mmol) was placed in a 1 L round bottom flask. 200 ml of THF was added, and the reaction mixture was stirred at −5° C. Potassium tert-butoxide (11 g, 98.256 mmol) dissolved in 110 mL THF was added dropwise under nitrogen protection. Then the mixture was stirred at 0° C. for 40 minutes. Ethyl bromoacetate (10.56 mL, 95.498 mmol) was added. The reaction was warmed to room temperature and stirred overnight. After the reaction is completed, 100 ml water was added, and THF was removed via concentration in vacuo. Then the mixture was transferred to a 1 L separatory funnel and 100 mL water was added, followed by extracting with ethyl acetate (100 mL*2). The combined organic phase was dried over anhydrous Na₂SO₄ and concentrated in vacuo. The residue was purified with silica gel column chromatography (PE:EA=90:10-65:35) to give target product (14.1 g, 64%). [M+H-Boc]⁺=192.0.

### Step 2: 2, 2-dimethyl-4-oxo-3, 8, 11-trioxa-5-azatridecan-13-oic Acid

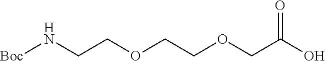 [SMILES: CC(C)(C)OC(=O)NCCOCCOCC(=O)O]

To a solution of ethyl 2, 2-dimethyl-4-oxo-3, 8, 11-trioxa-5-azatridecan-13-oate (3 g, 0.104 mmol) in 1, 4-dioxane (40 mL), was added lithium hydroxide (1.056 g, 0.250 mmol) with stirring, and water (20 mL) dropwise. The reaction mixture was stirred at R.T. over weekend. After the reaction was completed, the mixture was concentrated in vacuo to remove dioxane. The residue was added 50 mL water and extracted with EA: hexane=1:1 (200 mL*2). After liquid separation, hydrochloric acid (1 mol/L) was added dropwise to the aqueous phase until pH=1, and the aqueous phase was extracted with ethyl acetate (100 mL*4), and the combined organic phase was dried over anhydrous Na2SO₄, suction-filtered, then concentrated in vacuo to give the desired product (2.1 g, crude). [M+H-Boc]⁺=164.0.

### Step 3: tert-butyl (S)-(2-((5-(4-(3-carbamoyl-2-(4-phenoxyphenyl)-4, 5, 6, 7 tetrahydropyrazolo[1, 5-a]pyrimidin-7-yl)piperidin-1-yl)-5-oxopentyl)oxy)ethyl) carbamate

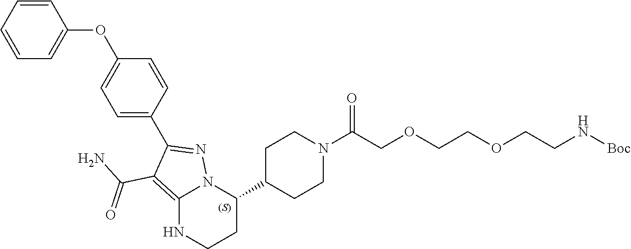

A mixture of 2, 2-dimethyl-4-oxo-3, 8, 11-trioxa-5-azatridecan-13-oic acid (189 mg, 0.720 mmol), HOBt (142 mg, 1.056 mmol), EDCI (202.7 mg, 1.056 mmol) and DIPEA (185 mg, 1.44 mmol) in DCM (10 mL) was stirred at room temperature for 5 min. Then (S)-2-(4-phenoxyphenyl)-7-(piperidin-4-yl)-4, 5, 6, 7-tetrahydropyrazolo[1,5-a]pyrimidine-3-carboxamide(200 mg, 0.480 mmol) was added. The mixture was stirred at room temperature overnight. After LCMS indicated that the reaction was completed, the reaction mixture was concentrated in vacuo. The residue was purified with silica gel column chromatography DCM:MeOH (100:090:10) to afford desired product (177 mg, 56%). [M+H]⁺=663.0.

### Step 4: (S)-7-(1-(2-(2-(2-aminoethoxy)ethoxy)acetyl)piperidin-4-yl)-2-(4-phenoxyphenyl)-4, 5, 6, 7-tetrahydropyrazolo[1,5-a]pyrimidine-3-carboxamide hydrochloride

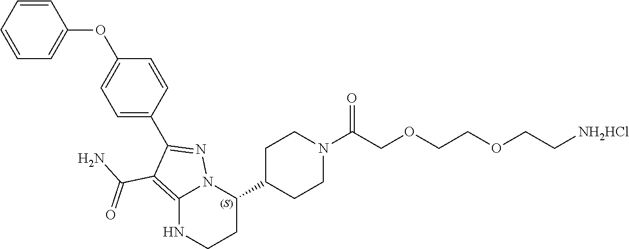

tert-butyl (S)-(2-((5-(4-(3-carbamoyl-2-(4-phenoxyphenyl)-4, 5, 6, 7-tetrahydropyrazolo[1, 5-a]pyrimidin-7-yl)piperidin-1-yl)-5-oxopentyl)oxy)ethyl) carbamate (600 mg, 0.916 mmol) was dissolved in 4M HCl in dioxane (10 mL). Then the mixture was stirred at room temperature for 2 h. After determined the reaction to be complete by LCMS, the reaction mixture was concentrated in vacuo to afford desired product (600 mg, crude), which was used in the next step without further purification. [M+H]⁺=563.0.

### Step 5: tert-butyl 2-(4-(2-(2, 6-dioxopiperidin-3-yl)-1, 3-dioxoisoindolin-5-yl)piperazin-1-yl)acetate

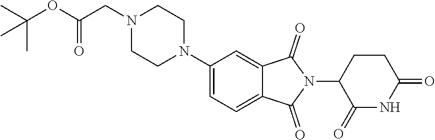 [SMILES: CC(C)(C)OC(=O)CN1CCN(c2ccc3c(c2)C(=O)N(C2CCC(=O)NC2=O)C3=O)CC1]

A mixture of 2-(2, 6-dioxopiperidin-3-yl)-5-(piperazin-1-yl)isoindoline-1, 3-dione (200 mg, 0.585 mmol), tert-butyl 2-bromoacetate (148 mg, 0.760 mmol) and DIPEA (150 mg, 1.17 mmol) in MeCN was stirred at 70° C. overnight. After LCMS indicated that the reaction was completed, the mixture was cooled, and then concentrated in vacuo. The residue was purified with silica gel column chromatography (DCM:MeOH=100:0-95:5) to afford desired product (100 mg, crude). [M+H]⁺=457.0.

### Step 6: 2-(4-(2-(2, 6-dioxopiperidin-3-yl)-1, 3-dioxoisoindolin-5-yl)piperazin-1-yl)acetic Acid

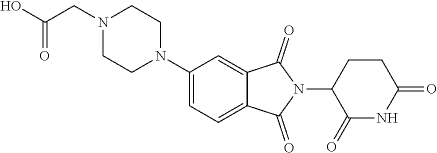 [SMILES: O=C(O)CN1CCN(c2ccc3c(c2)C(=O)N(C2CCC(=O)NC2=O)C3=O)CC1]

A solution of tert-butyl 2-(4-(2-(2, 6-dioxopiperidin-3-yl)-1, 3-dioxoisoindolin-5-yl)piperazin-1-yl)acetate(100 mg, 0.219 mmol) in TFA(8 mL) was stirred at room temperature for 2 h. After LCMS indicated that the reaction was completed, the mixture was concentrated in vacuo to afford desired product (100 mg, crude), which was used in the next step without further purification. [M+H]⁺=401.0.

### Step 7: (7S)-7-(1-(2-(2-(2-(2-(4-(2-(2, 6-dioxopiperidin-3-yl)-1, 3-dioxoisoindolin-5-yl)piperazin-1-yl)acetamido)ethoxy)ethoxy)acetyl)piperidin-4-yl)-2-(4-phenoxyphenyl)-4, 5, 6, 7-tetrahydropyrazolo[1,5-a]pyrimidine-3-carboxamide

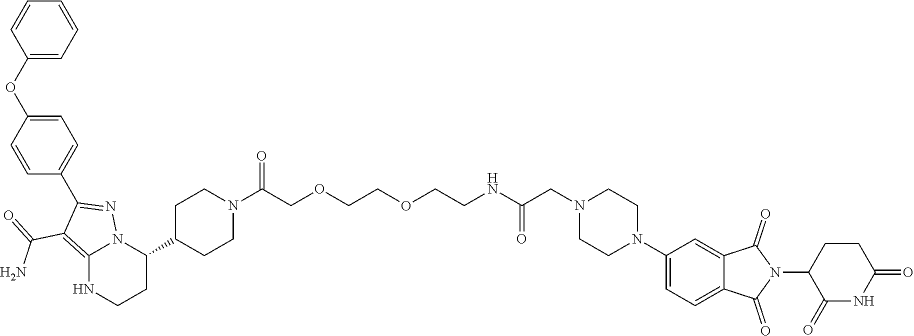 [SMILES: NC(=O)c1c(-c2ccc(Oc3ccccc3)cc2)nn2c1NCC[C@H]2C1CCN(C(=O)COCCOCCNC(=O)CN2CCN(c3ccc4c(c3)C(=O)N(C3CCC(=O)NC3=O)C4=O)CC2)CC1]

A mixture of (S)-7-(1-(2-(2-(2-aminoethoxy)ethoxy)acetyl)piperidin-4-yl)-2-(4-phenoxyphenyl)-4, 5, 6, 7-tetrahydropyrazolo[1,5-a]pyrimidine-3-carboxamide hydrochloride (60 mg, 0.1 mmol), 2-(4-(2-(2, 6-dioxopiperidin-3-yl)-1, 3-dioxoisoindolin-5-yl)piperazin-1-yl)acetic acid (40 mg, 0.1 mmol), HATU (76 mg, 0.2 mmol) and DIPEA (38 mg, 0.3 mmol) in DCM (5 mL) was stirred at room temperature over weekend. After LCMS indicated that the reaction was completed, the mixture was concentrated in vacuo, and the residue was purified with pre-TLC (DCM:MeOH=15:1) to afford the crude product. Then the crude product was purified with pre-HPLC to afford desired product (36.67 mg, 38.8%). ¹H NMR (400 MHz, DMSO) δ_{H} 11.08 (s, 1H), 7.79 (s, 1H), 7.66 (d, J=12.0 Hz, 1H), 7.50 (d, J=12.0 Hz, 2H), 7.41 (t, J=8.0 Hz, 2H), 7.34 (s, 1H), 7.25 (d, J=12.0 Hz, 1H), 7.17 (t, J=8.0 Hz, 1H), 7.12-7.01 (m, 3H), 6.67 (s, 1H), 4.99 (s, 1H), 4.36 (d, J=12.0 Hz, 1H), 4.12 (dd, J=32.0, 12.0 Hz, 2H), 3.99 (s, 1H), 3.79 (s, 1H), 3.60-3.39 (m, 9H), 3.31-3.23 (m, 4H), 2.91-2.70 (m, 3H), 2.56 (s, 5H), 2.21 (s, 1H), 2.09-1.80 (m, 3H), 1.68 (s, 1H), 1.51 (s, 1H), 1.41-1.05 (m, 3H); [M+H]⁺=944.8.

### Example 42: (7S)-7-(1-(2-(2-(2-(4-(4-(2-(2, 6-dioxopiperidin-3-yl)-1, 3-dioxoisoindolin-5-yl)piperazin-1-yl)butanamido)ethoxy)ethoxy)acetyl)piperidin-4-yl)-2-(4-phenoxyphenyl)-4, 5, 6, 7-tetrahydropyrazolo[1,5-a]pyrimidine-3-carboxamide

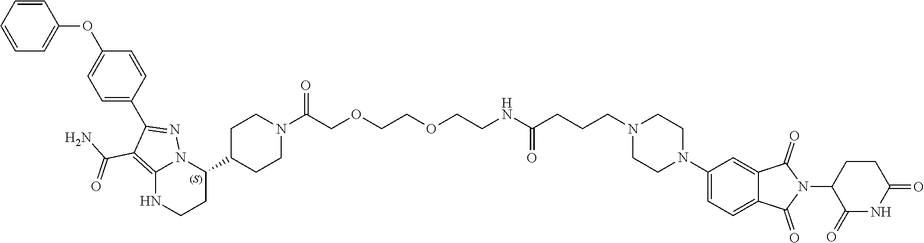

The titled compound was synthesized in the procedures similar to Example 23. ¹H NMR (400 MHz, DMSO) δ_{H} 11.08 (s, 1H), 7.85 ((t, J=4.0 Hz, 1H)), 7.67 (d, J=8.0 Hz, 1H), 7.50 (d, J=8.0 Hz, 2H), 7.41 (t, J=8.0 Hz, 2H), 7.33 (d, J=1.6 Hz, 1H), 7.24 (dd, J=8.0 Hz, 1.6 Hz, 1H), 7.18 (t, J=8.0 Hz, 1H), 7.11-7.01 (m, 4H), 6.68 (s, 1H), 5.07 (dd, J=12.0, 4.0 Hz, 1H), 4.38 (d, J=12.0 Hz, 1H), 4.12 (dd, J=32.0 Hz, 12.0 Hz, 2H), 4.00 (s, 1H), 3.81 (s, 1H), 3.58-3.48 (m, 4H), 3.45-3.36 (m, 6H), 3.30 (s, 2H), 3.18 (dd, J=12.0 Hz, 4.0 Hz, 2H), 2.96-2.84 (m, 2H), 2.64-2.53 (m, 2H), 2.46 (s, 4H), 2.30-2.16 (m, 3H), 2.09 (t, J=8.0 Hz, 2H), 2.05-1.81 (m, 3H), 1.74-1.62 (m, 3H), 1.53 (t, J=12.0 Hz, 1H), 1.41-1.26 (m, 1H)), 1.20-1.01 (m, 1H); [M+H]⁺=972.9.

### Example 43: (7S)-7-(1′-(4-((2-((2-(2, 6-dioxopiperidin-3-yl)-1, 3-dioxoisoindolin-4-yl)amino)ethyl)carbamoyl)benzyl)-[1, 4′-bipiperidin]-4-yl)-2-(4-phenoxyphenyl)-4, 5, 6, 7-tetrahydropyrazolo[1,5-a]pyrimidine-3-carboxamide

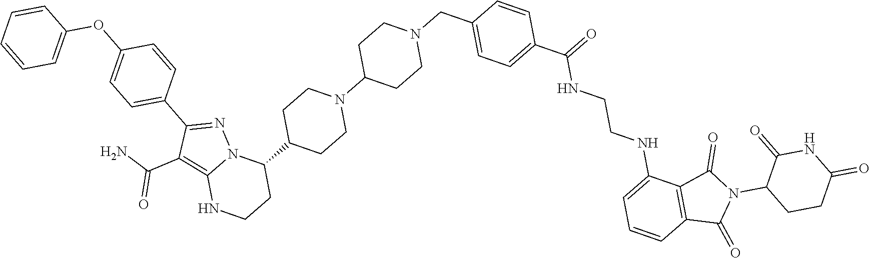 [SMILES: NC(=O)c1c(-c2ccc(Oc3ccccc3)cc2)nn2c1NCC[C@H]2C1CCN(C2CCN(Cc3ccc(C(=O)NCCNc4cccc5c4C(=O)N(C4CCC(=O)NC4=O)C5=O)cc3)CC2)CC1]

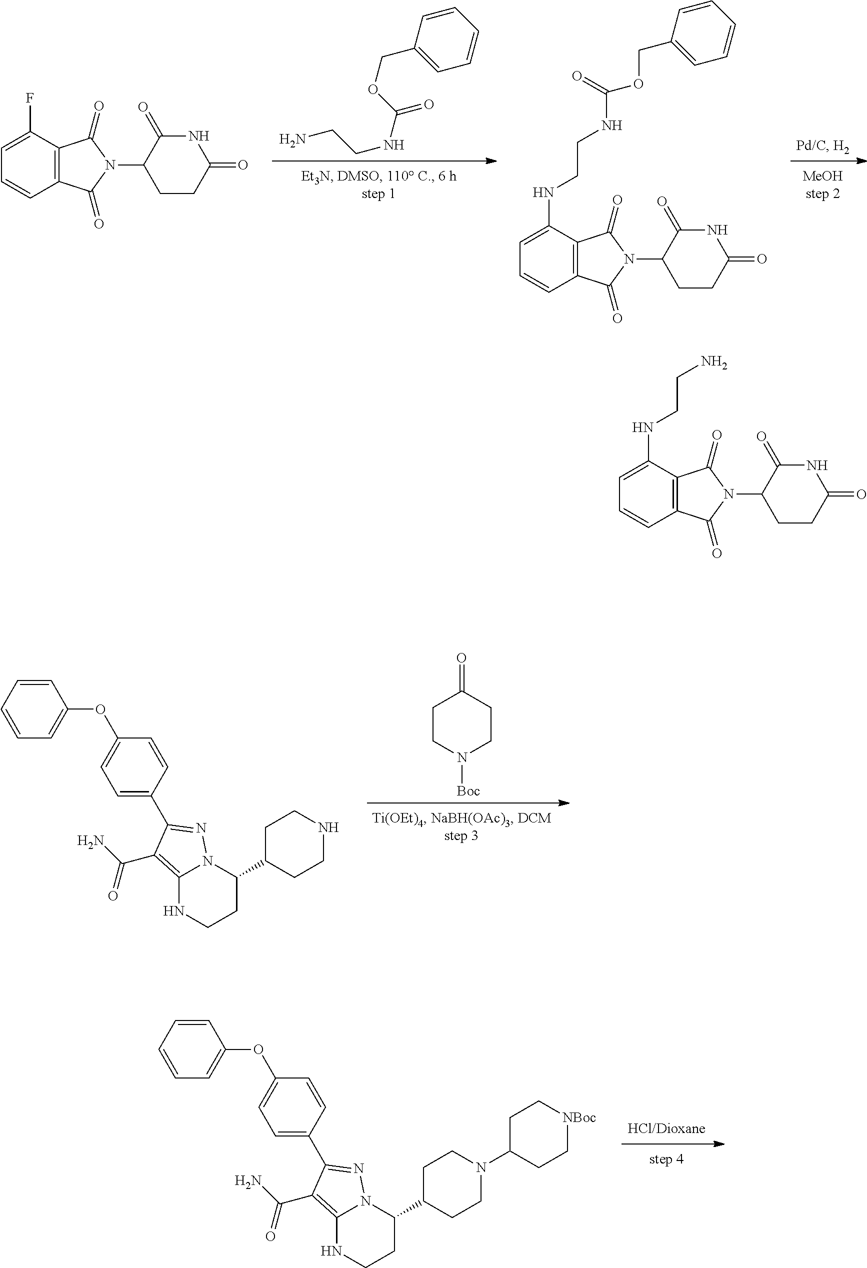

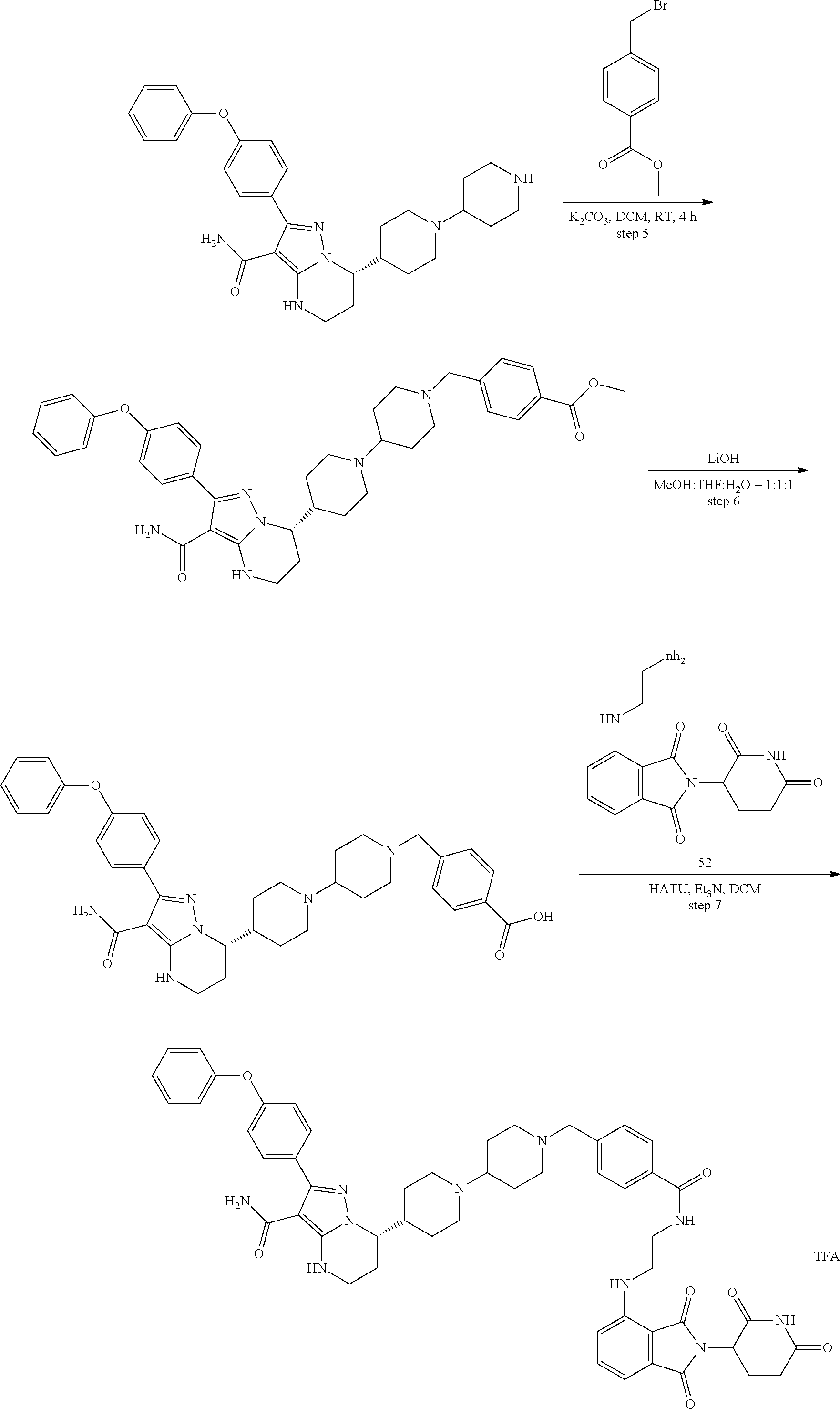

### Step 1: Benzyl (2-((2-(2, 6-dioxopiperidin-3-yl)-1, 3-dioxoisoindolin-4-yl)amino) ethyl)carbamate

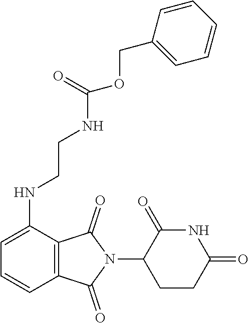 [SMILES: O=C1CCC(N2C(=O)c3cccc(NCCNC(=O)OCc4ccccc4)c3C2=O)C(=O)N1]

To a mixture of 2-(2, 6-dioxopiperidin-3-yl)-4-fluoroisoindoline-1, 3-dione (276 mg, 1 mmol) and benzyl (2-aminoethyl)carbamate (194 mg, 1 mmol) dissolved in DMSO (5 mL) was added Et₃N (303 mg, 3 mmol). The mixture was stirred at 110° C. for 6 hours. Then, the mixture was cooled to R.T. and washed with water (25 mL). The mixture was extracted with DCM (30 mL×3). The organic layers were combined and washed with saturated NaCl aq., dried over Na₂SO₄ and filtered. The filtrate was concentrated in vacuum. The residue was purified with silica gel column chromatography (DCM:MeOH=100/0 to 20/1) to afford benzyl (2-((2-(2, 6-dioxopiperidin-3-yl)-1, 3-dioxoisoindolin-4-yl)amino)ethyl)carbamate (410 mg, 91%). [M+H]⁺=450.8.

### Step 2: 4-((2-aminoethyl)amino)-2-(2, 6-dioxopiperidin-3-yl)isoindoline-1, 3-dione

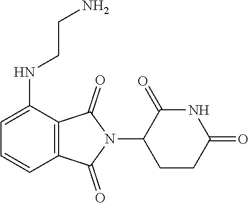 [SMILES: NCCNc1cccc2c1C(=O)N(C1CCC(=O)NC1=O)C2=O]

To a mixture of benzyl (2-((2-(2, 6-dioxopiperidin-3-yl)-1, 3-dioxoisoindolin-4-yl)amino)ethyl)carbamate (410 mg, 0.91 mmol) dissolved in MeOH (50 ml) was added Pd/C (80 mg, 10 w.t. %). And the mixture was stirred at room temperature under one hydrogen balloon overnight. Then the mixture was filtered and concentrated to afford 4-((2-aminoethyl)amino)-2-(2, 6-dioxopiperidin-3-yl)isoindoline-1, 3-dione (220 mg, 76%). [M+H]⁺=316.8.

### Step 3: Tert-butyl (S)-4-(3-carbamoyl-2-(4-phenoxyphenyl)-4, 5, 6, 7-tetrahydropyrazolo[1, 5-a]pyrimidin-7-yl)-[1, 4′-bipiperidine]-1′-carboxylate

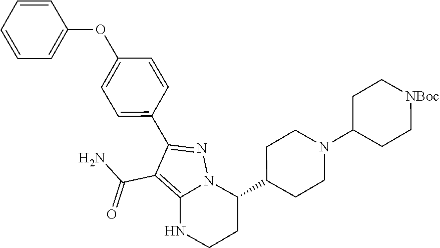 [SMILES: CC(C)(C)OC(=O)N1CCC(N2CCC([C@@H]3CCNc4c(C(N)=O)c(-c5ccc(Oc6ccccc6)cc5)nn43)CC2)CC1]

To a solution of compound (S)-2-(4-phenoxyphenyl)-7-(piperidin-4-yl)-4, 5, 6, 7-tetrahydropyrazolo[1,5-a]pyrimidine-3-carboxamide (418 mg, 1 mmol, 1.00 eq) and tert-butyl 4-oxopiperidine-1-carboxylate (199 mg, 1 mmol, 1.00 eq) dissolved in DCM (10 ml) were added tetraethyl titanate (913 mg, 4 mmol). The mixture was stirred at room temperature overnight. Then, sodium triacetoxyborohydride (848 mg, 4 mmol) was added to the reaction. The solution was quenched with Na2SO_{4.10} H₂O and extracted with DCM (25 mL). The mixture was filtered, and the filtrate was concentrated in vacuum. The residue was purified with silica gel column chromatography (DCM:MeOH=100/0 to 20/1) to afford tert-butyl (S)-4-(3-carbamoyl-2-(4-phenoxyphenyl)-4, 5, 6, 7-tetrahydropyrazolo[1,5-a]pyrimidin-7-yl)-[1, 4′-bipiperidine]-1′-carboxylate(500 mg, 83%). [M+H]⁺=600.9.

### Step 4: (S)-7-([1, 4′-bipiperidin]-4-yl)-2-(4-phenoxyphenyl)-4, 5, 6, 7tetrahydropyrazolo[1, 5-a]pyrimidine-3-carboxamide

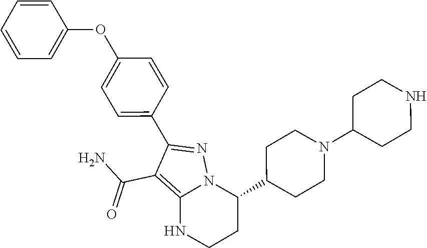 [SMILES: NC(=O)c1c(-c2ccc(Oc3ccccc3)cc2)nn2c1NCC[C@H]2C1CCN(C2CCNCC2)CC1]

To a mixture of tert-butyl (S)-4-(3-carbamoyl-2-(4-phenoxyphenyl)-4, 5, 6, 7-tetrahydropyrazolo[1,5-a]pyrimidin-7-yl)-[1, 4′-bipiperidine]-1′-carboxylate (500 mg, 0.83 mmol) dissolved in 1, 4-dioxane (6 ml) was added HCl/dioxane (4N, 2.1 mL). The mixture was stirred at R.T. for 4h. Solvent was removed under reduced pressure. The residue pH value was adjusted to 9-11 with saturate NaHCO₃aq. The mixture was extracted with DCM (15 mL×3). The organic layers were combined and dried over Na₂SO₄. The mixture was filtered and concentrated in vacuum to afford (S)-7-([1, 4′-bipiperidin]-4-yl)-2-(4-phenoxyphenyl)-4, 5, 6, 7-tetrahydropyrazolo[1,5-a] pyrimidine-3-carboxamide (408 mg, 98%). [M+H]⁺=500.8.

### Step 5: methyl (S)-4-((4-(3-carbamoyl-2-(4-phenoxyphenyl)-4, 5, 6, 7-tetrahydropyrazolo[1, 5-a]pyrimidin-7-yl)-[1, 4′-bipiperidin]-1′-yl)methyl)benzoate

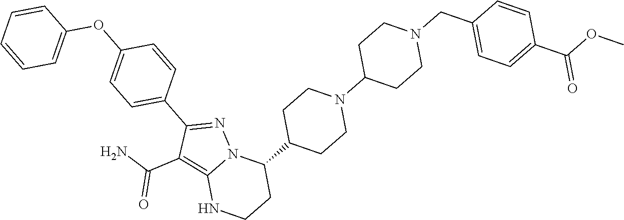 [SMILES: COC(=O)c1ccc(CN2CCC(N3CCC([C@@H]4CCNc5c(C(N)=O)c(-c6ccc(Oc7ccccc7)cc6)nn54)CC3)CC2)cc1]

A mixture of (S)-7-([1, 4′-bipiperidin]-4-yl)-2-(4-phenoxyphenyl)-4, 5, 6, 7-tetrahydropyrazolo[1,5-a] pyrimidine-3-carboxamide (408 mg, 0.81 mmol) in DCM (10 mL) was added to Et₃N (245 mg, 2.43 mmol) and methyl 4-(bromomethyl)benzoate (228 mg, 1 mmol). The reaction was stirred at R.T. for 3 h. The mixture was washed with water (15 mL) and extracted with DCM (20 mL×3). The organic layers were combined and dried over Na₂SO₄. The mixture was filtered and the filtrate was concentrated in vacuum to afford methyl (S)-4-((4-(3-carbamoyl-2-(4-phenoxyphenyl)-4, 5, 6, 7-tetrahydropyrazolo[1,5-a]pyrimidin-7-yl)-[1, 4′-bipiperidin]-1′-yl)methyl)benzoate (400 mg, 76%). [M+H]⁺=648.8.

### Step 6: (S)-4-((4-(3-carbamoyl-2-(4-phenoxyphenyl)-4, 5, 6, 7-tetrahydropyrazolo[1,5-a] pyrimidin-7-yl)-[1, 4′-bipiperidin]-1′-yl)methyl)benzoic Acid

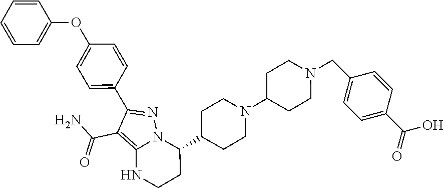 [SMILES: NC(=O)c1c(-c2ccc(Oc3ccccc3)cc2)nn2c1NCC[C@H]2C1CCN(C2CCN(Cc3ccc(C(=O)O)cc3)CC2)CC1]

To a mixture of methyl (S)-4-((4-(3-carbamoyl-2-(4-phenoxyphenyl)-4, 5, 6, 7-tetrahydropyrazolo[1,5-a]pyrimidin-7-yl)-[1, 4′-bipiperidin]-1′-yl)methyl)benzoate (400 mg, 0.62 mmol) dissolved in MeOH (3 ml)/THF (3 mL)/H₂O (3 mL) was added LiOH (74 mg, 3.1 mmol). The mixture was stirred at room temperature over overnight. Solvent was removed under reduced pressure. The residue was purified with reversed-phase column (Water: ACN=20/1 to 3/1) to obtain (S)-4-((4-(3-carbamoyl-2-(4-phenoxyphenyl)-4, 5, 6, 7-tetrahydropyrazolo[1,5-a]pyrimidin-7-yl)-[1, 4′-bipiperidin]-1′-yl)methyl)benzoic acid (120 mg, 31%). [M+H]⁺=634.8.

### Step 7: (7S)-7-(1′-(4-((2-((2-(2, 6-dioxopiperidin-3-yl)-1, 3-dioxoisoindolin-4-yl)amino) ethyl)carbamoyl)benzyl)-[1, 4′-bipiperidin]-4-yl)-2-(4-phenoxyphenyl)-4, 5, 6, 7-tetrahydropyrazolo[1,5-a]pyrimidine-3-carboxamide-2, 2, 2-trifluoroacetaldehyde trifluoroacetate

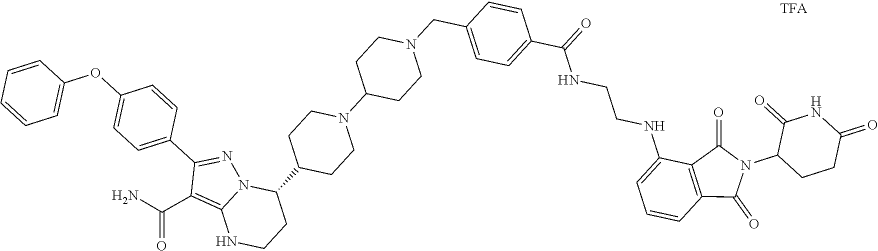 [SMILES: NC(=O)c1c(-c2ccc(Oc3ccccc3)cc2)nn2c1NCC[C@H]2C1CCN(C2CCN(Cc3ccc(C(=O)NCCNc4cccc5c4C(=O)N(C4CCC(=O)NC4=O)C5=O)cc3)CC2)CC1]

To a solution of (S)-4-((4-(3-carbamoyl-2-(4-phenoxyphenyl)-4, 5, 6, 7-tetrahydropyrazolo[1,5-a]pyrimidin-7-yl)-[1, 4′-bipiperidin]-1′-yl)methyl)benzoic acid (100 mg, 0.16 mmol, 1.00 eq) in DCM (5 mL) was added HATU (72 mg, 0.19 mmol). The mixture was stirred at R.T. for 10 min. Then, Et₃N (51 mg, 0.48 mmol) and 4-((2-aminoethyl)amino)-2-(2, 6-dioxopiperidin-3-yl)isoindoline-1, 3-dione (72 mg, 0.16 mmol) were added to the mixture. The reaction was stirred at R.T. for 2 h. The mixture was washed with water (10 mL) and extracted with DCM (10 mL×3). The organic layers were combined and dried over Na₂SO₄. The mixture was filtered, and the filtrate was concentrated in vacuum. The residue was purified with pre-HPLC to afford the title product(15 mg, 9%). ¹H NMR (400 MHz, DMSO) δ_{H} 11.10 (s, 1H), 8.79 (s, 1H), 7.92 (d, J=7.4 Hz, 2H), 7.58 (t, J=7.7 Hz, 3H), 7.50 (d, J=8.3 Hz, 2H), 7.42 (t, J=7.8 Hz, 2H), 7.25 (d, J=8.6 Hz, 1H), 7.18 (t, J=7.3 Hz, 1H), 7.11-6.99 (m, 5H), 6.80-6.74 (m, 2H), 5.05 (dd, J=12.6, 5.2 Hz, 1H), 4.34 (s, 2H), 4.05 (s, 2H), 3.48-3.45 (m, 3H), 3.32 (s, 4H), 2.97-2.87 (m, 6H), 2.69-2.54 (m, 2H), 2.26 (d, J=51.1 Hz, 4H), 2.06-1.74 (m, 8H), 1.64 (d, J=12.3 Hz, 2H); [M+H]⁺=932.9.

### Example 44: (7S)-7-(1-(4-(4-(4-(2-(2, 6-dioxopiperidin-3-yl)-1, 3-dioxoisoindolin-5-yl)piperazin-1-yl)phenyl)butanoyl)piperidin-4-yl)-2-(4-phenoxyphenyl)-4, 5, 6, 7-tetrahydropyrazolo[1, 5-a]pyrimidine-3-carboxamide

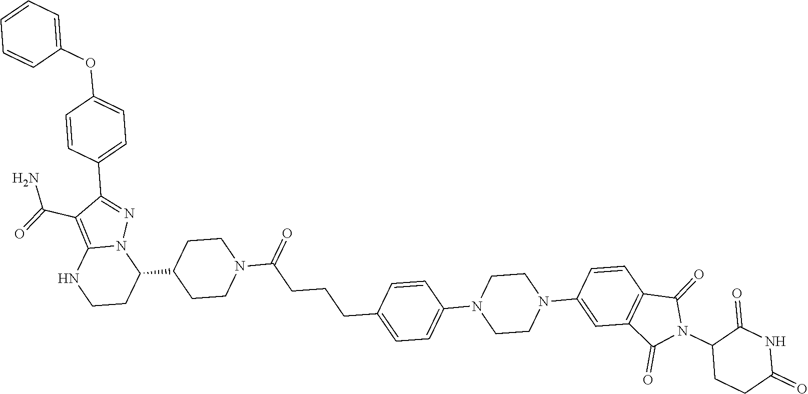 [SMILES: NC(=O)c1c(-c2ccc(Oc3ccccc3)cc2)nn2c1NCC[C@H]2C1CCN(C(=O)CCCc2ccc(N3CCN(c4ccc5c(c4)C(=O)N(C4CCC(=O)NC4=O)C5=O)CC3)cc2)CC1]

The titled compound was synthesized in the procedures similar to Example 40. ¹H NMR (400 MHz, DMSO) δ_{H} 10.82 (s, 1H), 7.68 (d, J=8.5 Hz, 1H), 7.50 (d, J=8.2 Hz, 2H), 7.38 (dd, J=16.7, 8.9 Hz, 3H), 7.28 (d, J=8.6 Hz, 1H), 7.15 (t, J=7.5 Hz, 1H), 7.05 (t, J=6.9 Hz, 6H), 6.89 (d, J=8.2 Hz, 2H), 5.88 (s, 2H), 5.03 (dd, J=12.5, 5.3 Hz, 1H), 4.01 (d, J=5.8 Hz, 1H), 3.59 (s, 4H), 3.26 (s, 4H), 2.94-2.81 (m, 2H), 2.67-2.50 (m, 7H), 2.26 (dd, J=18.4, 11.0 Hz, 3H), 1.99 (dd, J=26.5, 5.8 Hz, 4H), 1.82-1.67 (m, 3H), 1.55 (d, J=12.4 Hz, 1H), 1.25 (s, 3H); [M+H]⁺=903.9.

### Example 45: (7S)-7-(1-(3-(4-(4-((2-((2-(2, 6-dioxopiperidin-3-yl)-1-oxoisoindolin-4-yl)amino)acetamido)methyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

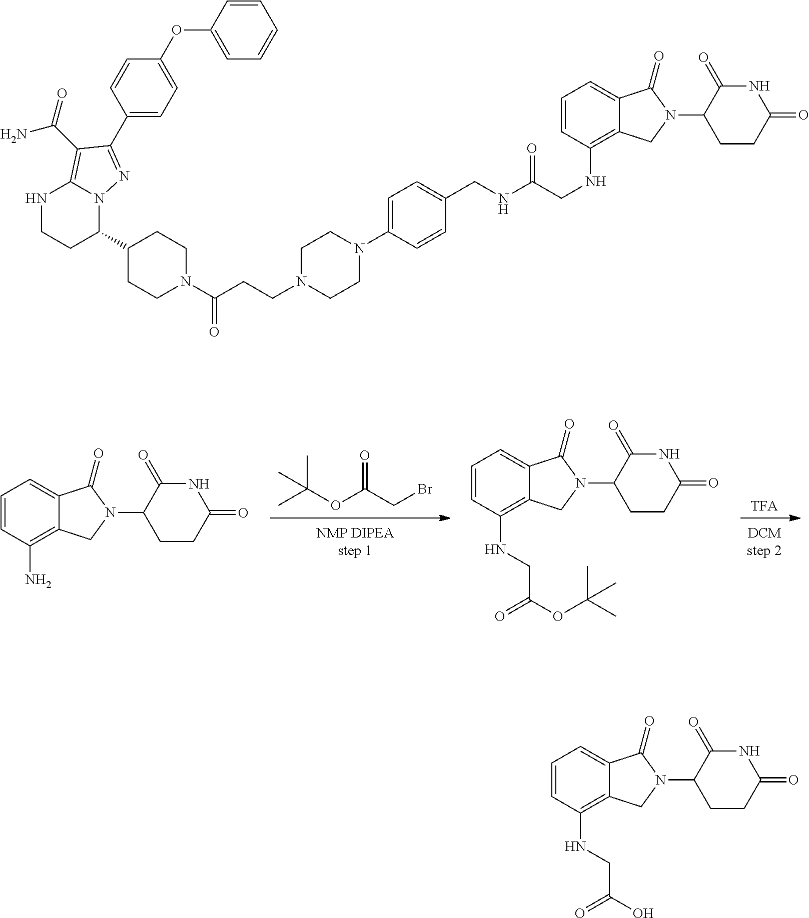

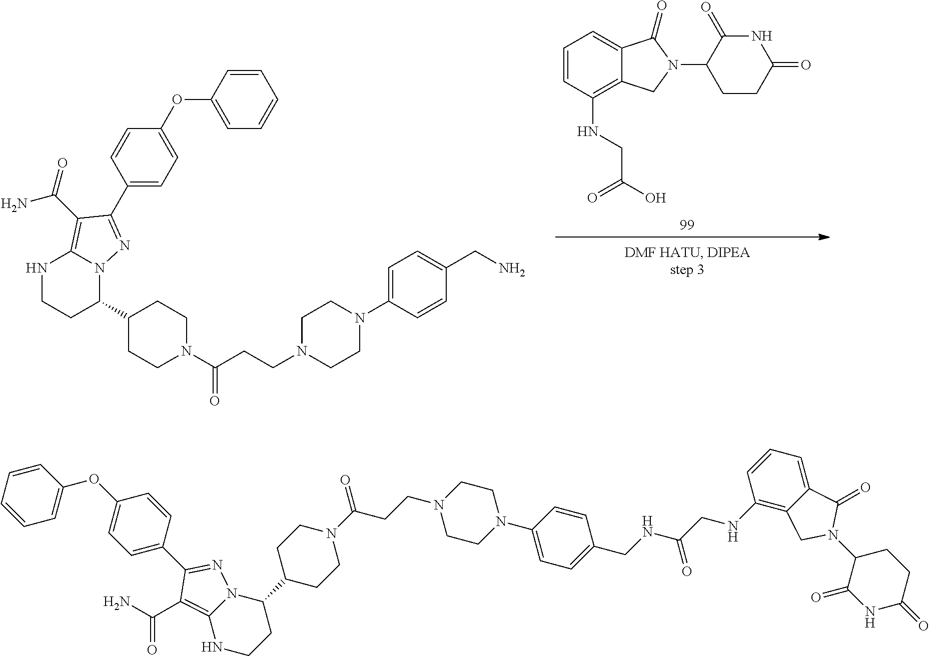

### Step 1: tert-butyl (2-(2, 6-dioxopiperidin-3-yl)-1-oxoisoindolin-4-yl)glycinate

A mixture of 3-(4-amino-1-oxoisoindolin-2-yl)piperidine-2, 6-dione (3 g, 11.58 mmol), tert-butyl 2-bromoacetate (2.5 g, 12.74 mmol) and DIPEA (5 g, 38.11 mmol) in NMP (20 mL) was stirred at 110° C. in a sealed tube for 8 h. LCMS showed the reaction was completed. Saturated aqueous NaCl was added to quench the reaction and the reaction mixture was extracted with DCM. The organic layer was dried over anhydrous Na₂SO₄ and evaporated in vacuum to afford the crude product, which was further purified with pre-TLC (DCM:MeOH=50:1) to give the product (0.4 g, 9.3%). [M+H]⁺=374.0.

### Step 2: (2-(2, 6-dioxopiperidin-3-yl)-1-oxoisoindolin-4-yl)glycine

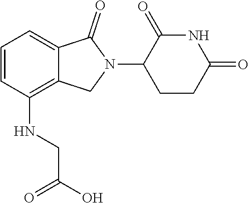 [SMILES: O=C(O)CNc1cccc2c1CN(C1CCC(=O)NC1=O)C2=O]

A mixture of tert-butyl (2-(2, 6-dioxopiperidin-3-yl)-1-oxoisoindolin-4-yl)glycinate (0.4 g, 1.07 mmol) and TFA (2 mL) in DCM (3 mL) was stirred at R.T. for 2 hours. LCMS showed the reaction was completed. The solvent was removed in vacuo to give the product (0.3 g, 88%), which was used for the next step without further purification. [M+H]⁺=318.0.

### Step 3: (7S)-7-(1-(3-(4-(4-((3-((2-(2, 6-dioxopiperidin-3-yl)-1-oxoisoindolin-4-yl)amino) acetamido)methyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

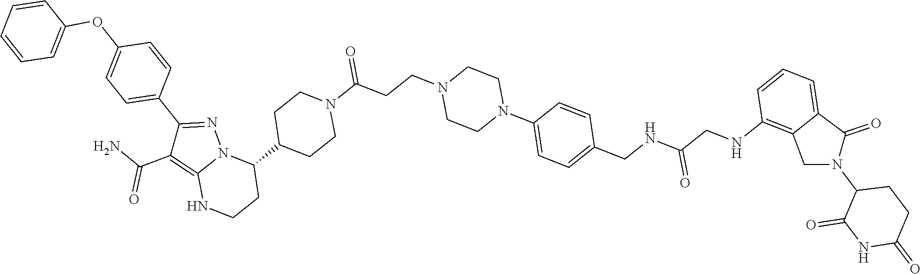 [SMILES: NC(=O)c1c(-c2ccc(Oc3ccccc3)cc2)nn2c1NCC[C@H]2C1CCN(C(=O)CCN2CCN(c3ccc(CNC(=O)CNc4cccc5c4CN(C4CCC(=O)NC4=O)C5=O)cc3)CC2)CC1]

A mixture of (2-(2, 6-dioxopiperidin-3-yl)-1-oxoisoindolin-4-yl)glycine (0.55 g, 0.79 mmol), (S)-7-(1-(3-(4-(4-(aminomethyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide (250 mg, 0.79 mmol), HATU (600 mg, 1.58 mmol) and DIPEA (31 mg, 0.239 mmol) in DMF (10 mL) was stirred at room temperature overnight. LCMS showed the reaction was completed. The solvent was evaporated in vacuum. The crude product thus obtained was further purified with pre-TLC (DCM: MeOH=10:1) to give the product (50 mg, 6.5%). ¹H NMR (400 MHz, DMSO) δ_{H} 11.01 (s, 1H), 8.38 (t, J=5.6 Hz, 1H), 7.50 (d, J=8.8 Hz, 2H), 7.42 (t, J=8.0 Hz, 2H), 7.29 (t, J=7.8 Hz, 1H), 7.17 (t, J=7.4 Hz, 1H), 7.11-7.03 (m, 6H), 6.99 (d, J=7.6 Hz, 1H), 6.83 (d, J=8.8 Hz, 2H), 6.68 (s, 1H), 6.59 (d, J=8.0 Hz, 1H), 6.11 (t, J=6.0 Hz, 1H), 5.12 (dd, J=13.2, 5.2 Hz, 1H), 4.43 (s, 1H), 4.32-4.14 (m, 5H), 4.04-3.91 (m, 2H), 3.79 (d, J=5.6 Hz, 2H), 3.30 (s, 2H), 3.17 (d, J=5.2 Hz, 1H), 3.06 (s, 4H), 2.97-2.88 (m, 3H), 2.68-2.55 (m, 7H), 2.37-2.25 (m, 3H), 2.18-1.85 (m, 4H), 1.75-1.62 (m, 2H), 1.66-1.52 (m, 1H), 1.36-1.10 (m, 7H); [M+H]⁺=961.9.

### Example 46: (7S)-7-(1-((E)-4-((5-((2-(2, 6-dioxopiperidin-3-yl)-1-oxoisoindolin-4-yl)amino)pentyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

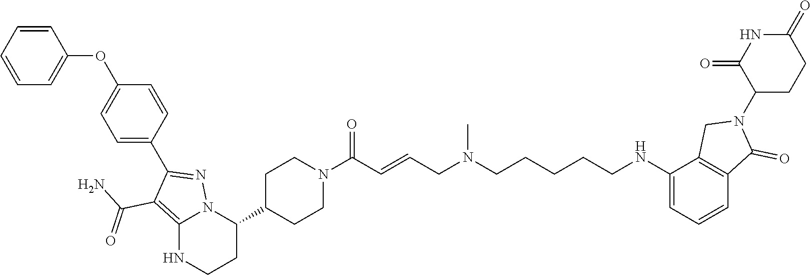 [SMILES: CN(C/C=C/C(=O)N1CCC([C@@H]2CCNc3c(C(N)=O)c(-c4ccc(Oc5ccccc5)cc4)nn32)CC1)CCCCCNc1cccc2c1CN(C1CCC(=O)NC1=O)C2=O]

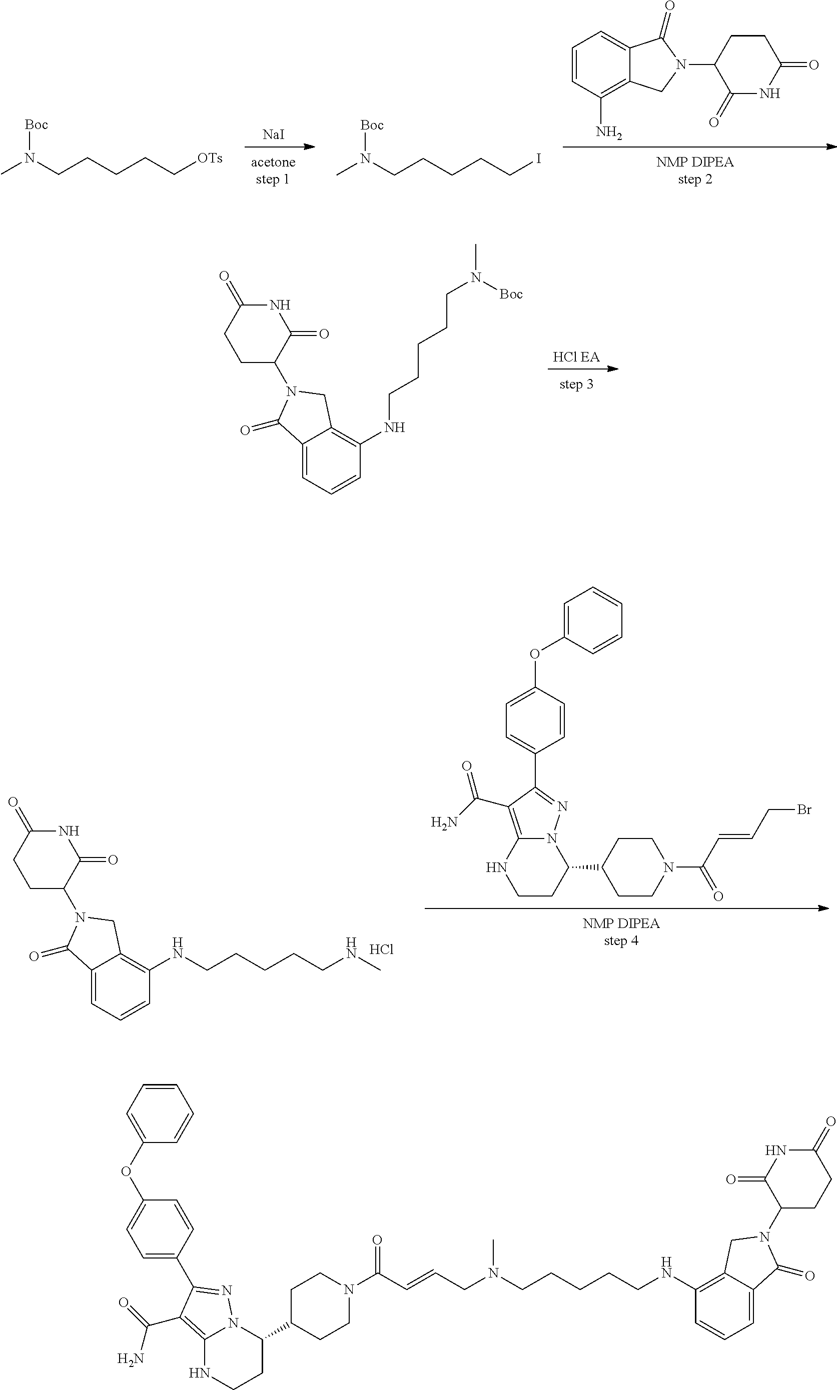

### Step 1: tert-butyl (5-iodopentyl)(methyl)carbamate

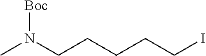

A mixture of 5-((tert-butoxycarbonyl)(methyl)amino)pentyl 4-methyl benzenesulfonate (0.9 g, 5.4 mmol) and NaI (1.344 g, 8.1 mmol) in acetone (20 mL) was stirred at 60° C. for 8 h. The solvent was evaporated under reduced pressure to give the product (0.9 g, crude), which was used in the next step without further purification.

### Step 2: tert-butyl (5-((2-(2, 6-dioxopiperidin-3-yl)-1-oxoisoindolin-4-yl)amino)pentyl) (methyl)carbamate

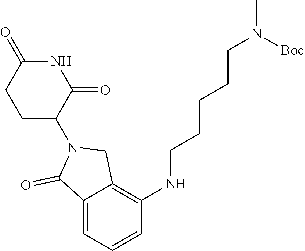 [SMILES: CN(CCCCCNc1cccc2c1CN(C1CCC(=O)NC1=O)C2=O)C(=O)OC(C)(C)C]

A mixture of 3-(4-amino-1-oxoisoindolin-2-yl)piperidine-2, 6-dione (0.71 g, 2.75 mmol), tert-butyl (5-iodopentyl)(methyl)carbamate (0.9 g, 2.75 mmol) and DIPEA (0.71 g, 5.5 mmol) in NMP (5 mL) was stirred at 110° C. overnight. LCMS showed the reaction was completed. The solvent was evaporated in vacuum to afford the crude product, then was added water (10 mL) and extracted with DCM (10 mL*3). The combined organic layer was dried over Na₂SO₄. Filtered and concentrated to give the crude product, which was purified with silica gel chromatography (MeOH: DCM=1:30-1:20) to give the product (1 g, 79%). [M+H]⁺=459.

### Step 3: 3-(4-((5-(methylamino)pentyl)amino)-1-oxoisoindolin-2-yl)piperidine-2, 6-dione

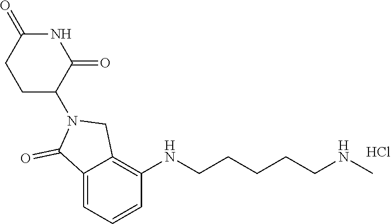 [SMILES: CNCCCCCNc1cccc2c1CN(C1CCC(=O)NC1=O)C2=O ; Cl]

A mixture of tert-butyl (5-((2-(2, 6-dioxopiperidin-3-yl)-1-oxoisoindolin-4-yl)amino)pentyl) (methyl)carbamate (1 g, 2.176 mmol) in EA (5 mL) was added dropwise EA/HCl (10 mL) and stirred at RT for 2 hour. LCMS showed the reaction was completed. The solvent was removed in vacuo to give the product (0.8 g, 90%), which was used for the next step without further purification. [M+H]⁺=359.

### Step 4: (7S)-7-(1-((E)-4-((5-((2-(2, 6-dioxopiperidin-3-yl)-1-oxoisoindolin-4-yl)amino) pentyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

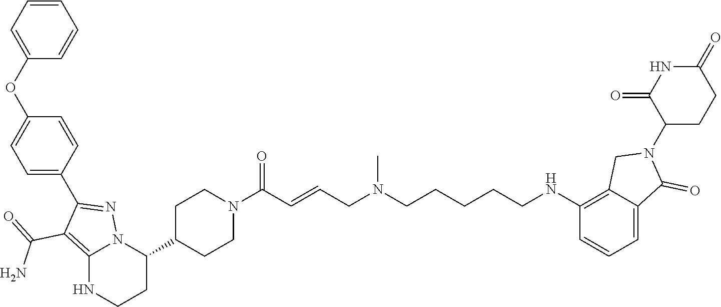 [SMILES: CN(C/C=C/C(=O)N1CCC([C@@H]2CCNc3c(C(N)=O)c(-c4ccc(Oc5ccccc5)cc4)nn32)CC1)CCCCCNc1cccc2c1CN(C1CCC(=O)NC1=O)C2=O]

A mixture of (S, E)-7-(1-(4-bromobut-2-enoyl)piperidin-4-yl)-2-(4-phenoxyphenyl)-4, 5, 6, 7-tetrahydropyrazolo[1,5-a]pyrimidine-3-carboxamide (0.1 g, 0.177 mmol), 3-(4-((5-(methylamino)pentyl)amino)-1-oxoisoindolin-2-yl)piperidine-2, 6-dione (70 mg, 0.177 mmol) and Et₃N (54 mg, 0.513 mmol) in CH₃CN (20 mL) was stirred at 80° C. under the atmosphere of N₂ overnight. LCMS showed the reaction was completed. The solvent was evaporated under reduced pressure and the residue was purified with pre-TLC to give the product (30 mg, 20%). ¹H NMR (400 MHz, DMSO) δ_{H} 11.01 (s, 1H), 7.49 (d, J=8.4 Hz, 2H), 7.42 (t, J=7.8 Hz, 2H), 7.28 (t, J=7.6 Hz, 1H), 7.17 (t, J=7.2 Hz, 1H), 7.07 (t, J=8.8 Hz, 4H), 6.92 (d, J=7.2 Hz, 1H), 6.75-6.55 (m, 4H), 5.61 (s, 1H), 5.11 (dd, J=13.2, 4.8 Hz, 1H), 4.53-4.43 (m, 1H), 4.27-3.73 (m, 6H), 3.15-2.85 (m, 11H), 2.79-2.57 (m, 6H), 2.33-2.24 (m, 6H), 2.12-1.83 (m, 6H), 1.84-1.51 (m, 8H), 1.45-1.30 (m, 4H); [M+H]⁺=842.

### Example 47: (7S)-7-(1-((E)-4-((6-((2-(2, 6-dioxopiperidin-3-yl)-1-oxoisoindolin-4-yl)amino)hexyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

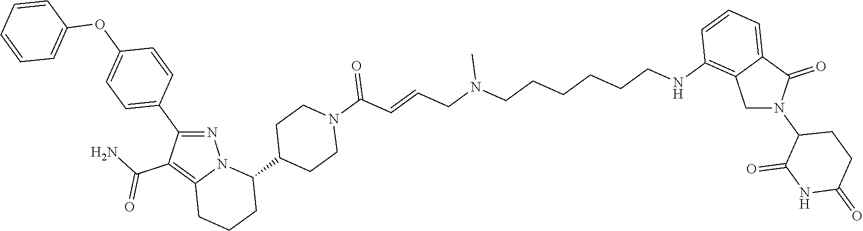 [SMILES: CN(C/C=C/C(=O)N1CCC([C@@H]2CCCc3c(C(N)=O)c(-c4ccc(Oc5ccccc5)cc4)nn32)CC1)CCCCCCNc1cccc2c1CN(C1CCC(=O)NC1=O)C2=O]

The titled compound was synthesized in the procedures similar to Example 53. ¹H NMR (400 MHz, DMSO) δ_{H} 11.01 (s, 1H), 7.49 (d, J=8.4 Hz, 2H), 7.42 (t, J=7.8 Hz, 2H), 7.28 (t, J=7.8 Hz, 1H), 7.17 (t, J=7.4 Hz, 1H), 7.07 (t, J=8.6 Hz, 4H), 6.92 (d, J=7.2 Hz, 1H), 6.78-6.56 (m, 4H), 5.59 (s, 1H), 5.11 (dd, J=13.2, 5.2 Hz, 1H), 4.50-4.40 (m, 1H), 4.27-3.74 (m, 6H), 3.15-2.85 (m, 9H), 2.76-2.57 (m, 5H), 2.38-2.19 (m, 6H), 2.10-1.85 (m, 5H), 1.81-1.52 (m, 8H), 1.45-1.26 (m, 8H); [M+H]⁺=855.9.

### Example 48: (7S)-7-(1-((E)-3-((2-(2-((2-(2, 6-dioxopiperidin-3-yl)-1, 3-dioxoisoindolin-4-yl)amino)ethoxy)ethyl)(methyl)amino)acryloyl)piperidin-4-yl)-2-(4-phenoxyphenyl)-4, 5, 6, 7-tetrahydropyrazolo[1,5-a] pyrimidine-3-carboxamide

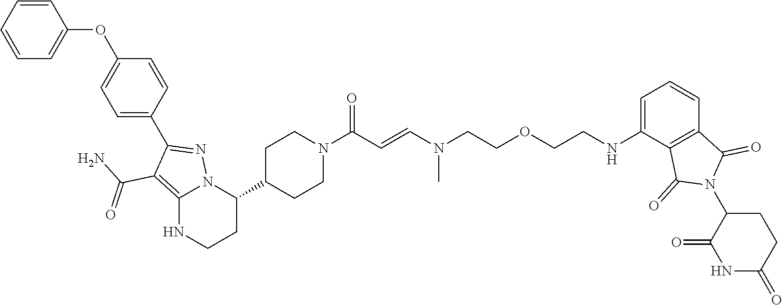 [SMILES: CN(/C=C/C(=O)N1CCC([C@@H]2CCNc3c(C(N)=O)c(-c4ccc(Oc5ccccc5)cc4)nn32)CC1)CCOCCNc1cccc2c1C(=O)N(C1CCC(=O)NC1=O)C2=O]

### Example 49: (7S)-7-(1-((E)-3-((2-((2-(2, 6-dioxopiperidin-3-yl)-1, 3-dioxoisoindolin-4-yl)amino)ethyl)(methyl)amino)acryloyl)piperidin-4-yl)-2-(4-phenoxyphenyl)-4, 5, 6, 7-tetrahydropyrazolo[1,5-a]pyrimidine-3-carboxamide

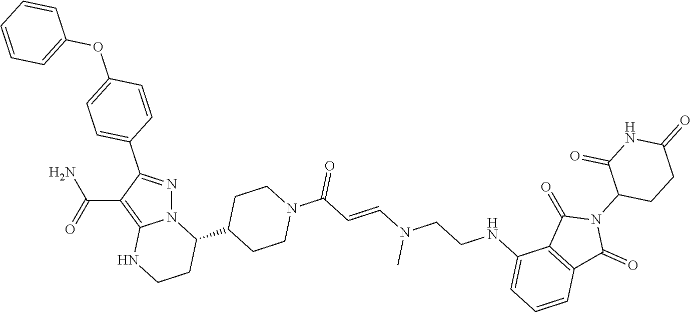 [SMILES: CN(/C=C/C(=O)N1CCC([C@@H]2CCNc3c(C(N)=O)c(-c4ccc(Oc5ccccc5)cc4)nn32)CC1)CCNc1cccc2c1C(=O)N(C1CCC(=O)NC1=O)C2=O]

### Example 50: (7S)-7-(1-((E)-4-((6-(3-(4-amino-1, 3-dioxoisoindolin-2-yl)-2, 6-dioxopiperidin-1-yl)hexyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

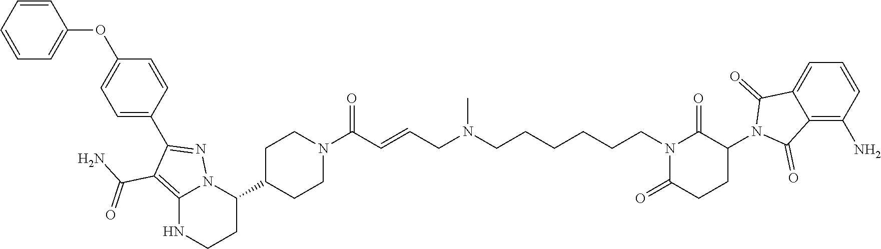 [SMILES: CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2C(=O)c3cccc(N)c3C2=O)C1=O]

The titled compound was synthesized in the procedures similar to Example 53. ¹H NMR (400 MHz, DMSO) δ_{H} 7.56-7.36 (m, 5H), 7.17 (t, J=7.0 Hz, 1H), 7.04 (m, 6H), 6.67 (s, 1H), 6.56-6.53 (m, 4H), 5.12 (d, J=8.0 Hz, 1H), 4.45 (s, 1H), 4.06-4.01 (m, 2H), 3.62 (s, 2H), 3.29 (s, 2H), 3.06 (s, 2H), 3.02-2.90 (m, 2H), 2.78-2.71 (m, 1H), 2.30-2.18 (m, 3H), 2.10 (s, 3H), 2.06-1.95 (m, 2H), 1.95-1.85 (m, 1H), 1.78-1.64 (m, 1H), 1.57-1.54 (m, 1H), 1.46-1.32 (m, 4H), 1.32-1.18 (m, 6H); [M+H]⁺=869.9.

### Example 51: (7S)-7-(1-((E)-4-((6-(3-(4-amino-1-oxoisoindolin-2-yl)-2,6-dioxopiperidin-1-yl)hexyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

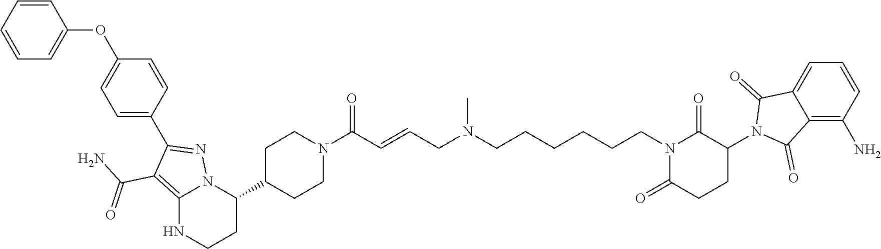 [SMILES: CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2C(=O)c3cccc(N)c3C2=O)C1=O]

The titled compound was synthesized in the procedures similar to Example 53. ¹H NMR (400 MHz, DMSO) δ_{H} 7.50 (d, J=8.0 Hz, 1H), 7.42 (t, J=7.6 Hz, 1H), 7.18 (d, J=6.8 Hz, 1H), 7.07 (t, J=8.8 Hz, 2H), 6.91 (d, J=7.2 Hz, 1H), 6.80 (d, J=7.6 Hz, 1H), 6.68 (s, 1H), 6.64-6.50 (m, 2H), 5.42 (s, 2H), 5.16 (d, J=13.6 Hz, 1H), 4.50-4.40 (m, 1H), 4.38-4.32 (m, 1H), 4.25-4.15 (m, 1H), 4.15-3.89 (m, 3H), 3.63-3.62 (m, 2H), 3.50-3.38 (m, 2H), 3.10-2.94 (m, 4H), 2.89-2.65 (m, 4H), 2.35-2.20 (m, 4H), 2.10-1.90 (m, 4H), 1.78-1.65 (m, 1H), 1.64-1.52 (m, 2H), 1.48-1.35 (m, 3H), 1.32-1.15 (s, 7H); [M+H]⁺=855.9.

### Example 52: (7S)-7-(1-((E)-1-(3-(4-amino-1-oxoisoindolin-2-yl)-2,6-dioxopiperidin-1-yl)-12-methyl-3, 6, 9-trioxa-12-azahexadec-14-en-16-oyl)piperidin-4-yl)-2-(4-phenoxyphenyl)-4, 5, 6, 7-tetrahydropyrazolo[1,5-a]pyrimidine-3-carboxamide

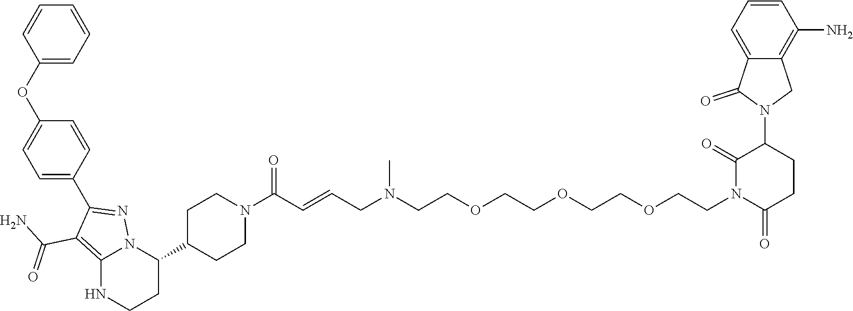 [SMILES: CN(C/C=C/C(=O)N1CCC([C@@H]2CCNc3c(C(N)=O)c(-c4ccc(Oc5ccccc5)cc4)nn32)CC1)CCOCCOCCOCCN1C(=O)CCC(N2Cc3c(N)cccc3C2=O)C1=O]

The titled compound was synthesized in the procedures similar to Example 53. ¹H NMR (400 MHz, DMSO) δ_{H} 7.50 (d, J=8.0 Hz, 2H), 7.42 (t, J=7.6 Hz, 2H), 7.18 (d, J=6.8 Hz, 2H), 7.07 (t, J=8.6 Hz, 4H), 6.91 (d, J=7.2 Hz, 1H), 6.80 (d, J=7.6 Hz, 1H), 6.68 (s, 1H), 6.60 (s, 2H), 5.43 (s, 2H), 5.17 (d, J=9.6 Hz, 1H), 4.47 (s, 1H), 4.36 (s, 1H), 4.25-4.16 (m, 1H), 4.14-3.97 (m, 3H), 3.84-3.79 (m, 4H), 3.53-3.43 (m, 14H), 3.12-2.90 (m, 3H), 2.83-2.61 (m, 4H), 2.37-2.16 (m, 4H), 2.10-1.82 (m, 4H), 1.78-1.50 (m, 3H), 1.35-1.10 (m, 4H); [M+H]⁺=931.9.

### Example 53: (7S)-7-(1-((E)-4-((5-(3-(4-amino-1-oxoisoindolin-2-yl)-2,6-dioxopiperidin-1-yl)pentyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

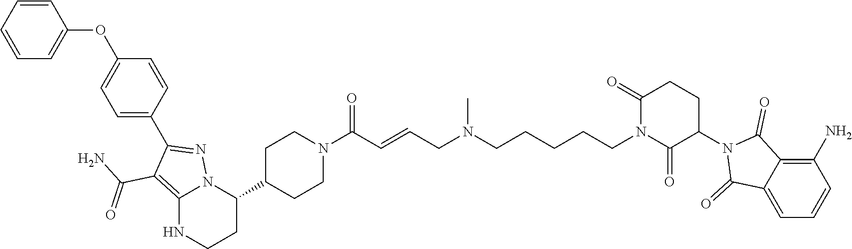 [SMILES: CN(C/C=C/C(=O)N1CCC([C@@H]2CCNc3c(C(N)=O)c(-c4ccc(Oc5ccccc5)cc4)nn32)CC1)CCCCCN1C(=O)CCC(N2C(=O)c3cccc(N)c3C2=O)C1=O]

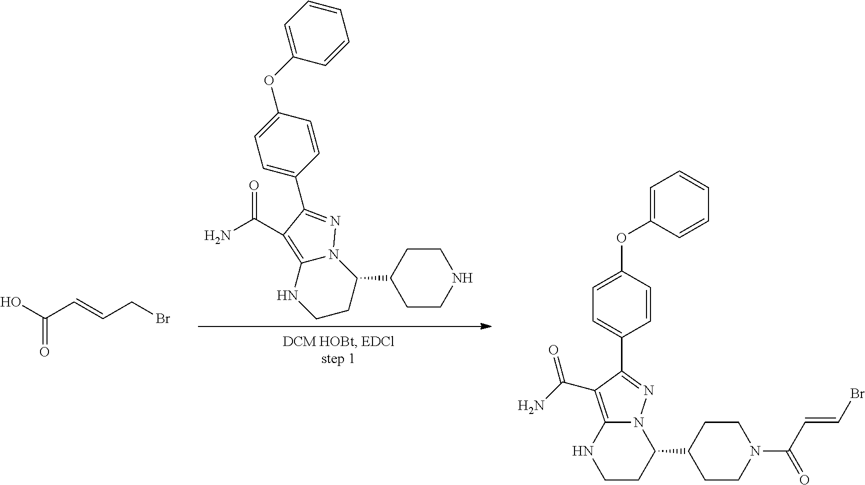 [SMILES: O=C(O)/C=C/CBr ; NC(=O)c1c(-c2ccc(Oc3ccccc3)cc2)nn2c1NCC[C@H]2C1CCNCC1 ; NC(=O)c1c(-c2ccc(Oc3ccccc3)cc2)nn2c1NCC[C@H]2C1CCN(C(=O)/C=C/Br)CC1]

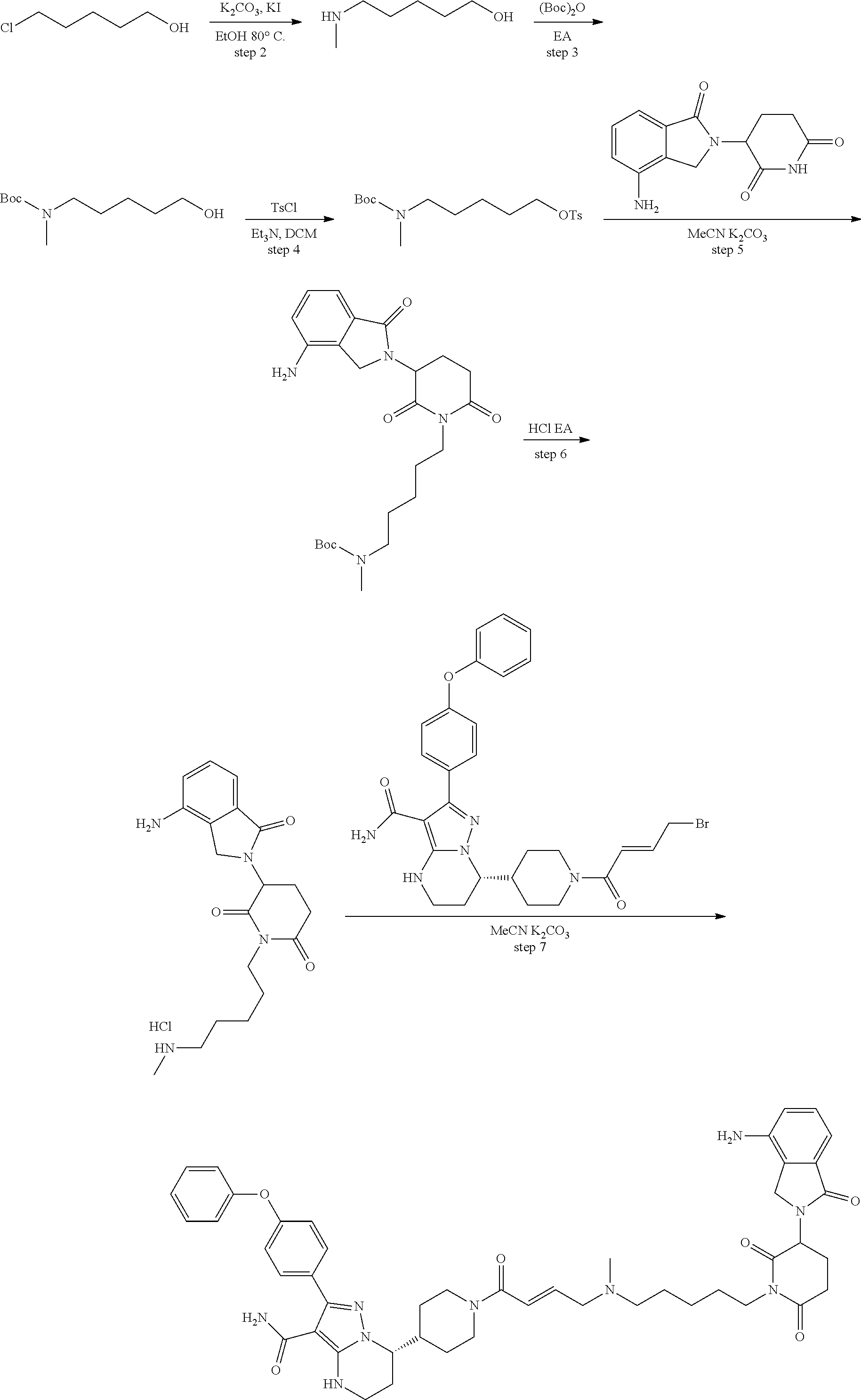

### Step 1: (S, E)-7-(1-(3-bromoacryloyl)piperidin-4-yl)-2-(4-phenoxyphenyl)-4, 5, 6, 7-tetrahydropyrazolo[1,5-a]pyrimidine-3-carboxamide

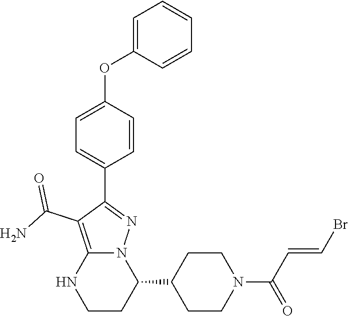 [SMILES: NC(=O)c1c(-c2ccc(Oc3ccccc3)cc2)nn2c1NCC[C@H]2C1CCN(C(=O)/C=C/Br)CC1]

A mixture of (E)-4-bromobut-2-enoic acid (0.22 g, 1.44 mmol), (S)-2-(4-phenoxyphenyl)-7-(piperidin-4-yl)-4, 5, 6, 7-tetrahydropyrazolo[1,5-a]pyrimidine-3-carboxamide (0.5 g, 1.2 mmol), HOBt (0.324 g, 2.4 mmol) and EDCI (0.46 mg, 2.4 mmol) in DCM (100 mL) was stirred at room temperature overnight. TLC (PE:EA=1:1, Rf=0.3) showed the reaction was completed. Then was added water 10 mL and extracted with DCM (10 mL*3). The combined organic layer was dried over Na₂SO₄. Filtered and concentrated to give the crude product, which was purified with silica gel chromatography (MeOH: DCM=1:30-1:10) to give the product (0.6 g, 90%). [M+H]⁺=550.0.

### Step 2: 5-(methylamino)pentan-1-ol

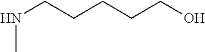

A mixture of 5-chloropentan-1-ol (16.4 g, 133.8 mmol), K₂CO₃ (37 g, 267.6 mmol) and KI (2.22 g, 13.38 mmol) in EtOH (CH₃NH₂ 30%) 80 mL was stirred at 70° C. in a sealed tube for 8 h. The reaction was monitored with TLC (stained by KMnO₄). The solvent was evaporated under reduced pressure to give the product (14 g, crude), which was used in the next step without further purification.

### Step 3: tert-butyl (5-hydroxypentyl)(methyl)carbamate

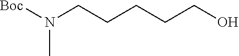

To a solution of 5-(methylamino)pentan-1-ol (10 g, 85.4 mmol) in EA (100 mL) was added dropwise (Boc)₂O (18.6 g, 85.4 mL) at 0° C. The mixture was stirred for 8 hours at room temperature. TLC (PE:EA=1:1, Rf=0.6) showed the reaction was completed. The solvent was evaporated under reduced pressure to give the product (16 g, crude), which was used in the next step without further purification.

### Step 4: 5-((tert-butoxycarbonyl)(methyl)amino)pentyl 4-methylbenzenesulfonate

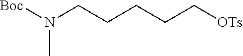 [SMILES: Cc1ccc(S(=O)(=O)OCCCCCN(C)C(=O)OC(C)(C)C)cc1]

To a solution of tert-butyl (5-hydroxypentyl)(methyl)carbamate (9 g, 41.5 mmol) and Et3N (8.4 g, 83 mmol) in DCM (100 mL) was added dropwise TsCl (7.9 g, 41.5 mmol) at 0° C. The mixture was stirred for 3 hours at room temperature. TLC (PE:EA=5:1, Rf=0.4) showed the reaction was completed. Then aqueous NH₄Cl was added and extracted with DCM (50 mL*3). The combined organic layer was dried over Na₂SO₄. Filtered and concentrated to give crude product, which was purified with silica gel chromatography (PE:EA=20:1-4 :1) to give the product (8 g, 51.2%). [M+H]⁺=372.0.

### Step 5: tert-butyl (5-(3-(4-amino-1-oxoisoindolin-2-yl)-2,6-dioxopiperidin-1-yl)pentyl)(methyl)carbamate

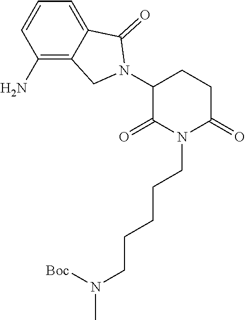 [SMILES: CN(CCCCCN1C(=O)CCC(N2Cc3c(N)cccc3C2=O)C1=O)C(=O)OC(C)(C)C]

A mixture of 5-((tert-butoxycarbonyl)(methyl)amino)pentyl 4-methylbenzenesulfonate (0.5 g, 1.3 mmol), 3-(4-amino-1-oxoisoindolin-2-yl)piperidine-2, 6-dione (0.34 g, 1.3 mmol) and K₂CO₃ (0.36 g, 2.6 mmol) in CH₃CN (20 mL) was stirred at 75° C. overnight. TLC (EA, Rf=0.2) showed the reaction was completed. Filtered and concentrated to give crude product, which was purified with silica gel chromatography (MeOH: DCM=1:30-1:10) to give the product (0.6 g, 70%). [M+H]⁺=459.0.

### Step 6: 3-(4-amino-1-oxoisoindolin-2-yl)-1-(5-(methylamino)pentyl)piperidine-2, 6-dione

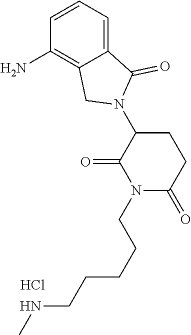 [SMILES: CNCCCCCN1C(=O)CCC(N2Cc3c(N)cccc3C2=O)C1=O ; Cl]

To a mixture of tert-butyl (5-((2-(2, 6-dioxopiperidin-3-yl)-1-oxoisoindolin-4-yl) amino) pentyl)(methyl)carbamate (200 mg, 0.41 mmol) in EA (2 mL) was added drop-wise EA/HCl (10 mL) and the mixture was stirred at RT for 2 hour. LCMS showed the reaction was completed. The solvent was removed in vacuo to give the product (150 mg, 90%), which was used for the next step without further purification. [M+H]⁺=359.0.

### Step 7: (75)-7-(1-((E)-4-((5-(3-(4-amino-1-oxoisoindolin-2-yl)-2,6-dioxopiperidin-1-yl)pentyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

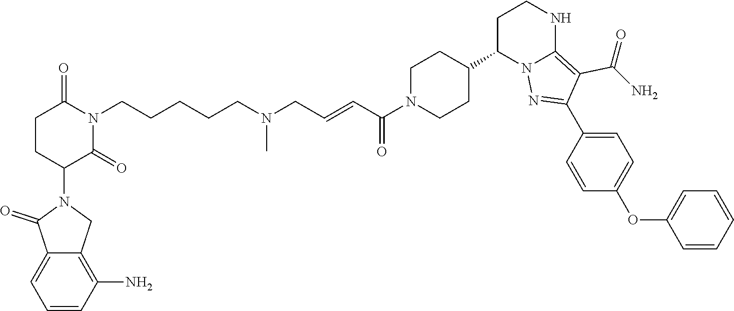 [SMILES: CN(C/C=C/C(=O)N1CCC([C@@H]2CCNc3c(C(N)=O)c(-c4ccc(Oc5ccccc5)cc4)nn32)CC1)CCCCCN1C(=O)CCC(N2Cc3c(N)cccc3C2=O)C1=O]

A mixture of (S, E)-7-(1-(4-bromobut-2-enoyl)piperidin-4-yl)-2-(4-phenoxyphenyl)-4, 5, 6, 7-tetrahydropyrazolo[1,5-a]pyrimidine-3-carboxamide (0.1 g, 0.177 mmol), oxoisoindolin-2-yl)-1-(5-(methylamino)pentyl)piperidine-2, 6-dione (74 mg, 0.177 mmol) and Et₃N (54 mg, 0.513 mmol) in CH₃CN (20 mL) was stirred at 80° C. under the atmosphere of N₂ overnight. LCMS showed the reaction was completed. The solvent was evaporated under reduced pressure and the residue was purified with pre-TCL to give the product (30 mg, 20%). ¹H NMR (400 MHz, DMSO) δ_{H} 7.50 (d, J=8.0 Hz, 1H), 7.41 (t, J=7.2 Hz, 1H), 7.18 (d, J=6.4 Hz, 1H), 7.07 (t, J=8.4 Hz, 2H), 6.91 (d, J=6.8 Hz, 1H), 6.80 (d, J=11.6 Hz, 1H), 6.68 (s, 1H), 6.63-6.52 (m, 1H), 5.43 (s, 1H), 5.15 (d, J=8.4 Hz, 1H), 4.50-4.40 (m, 1H), 4.35-4.18 (m, 1H), 4.15-3.97 (m, 2H), 3.70-3.55 (m, 1H), 3.10-2.90 (m, 2H), 2.82-2.63 (m, 2H), 2.35-2.15 (m, 1H), 2.09-1.83 (m, 2H), 179-1.54 (m, 2H), 1.45 (s, 1H), 1.35-1.15 (s, 3H); [M+H]⁺=842.0.

### Example 54: (7S)-7-(1-((E)-4-((3-(3-(4-amino-1-oxoisoindolin-2-yl)-2,6-dioxopiperidin-1-yl)propyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

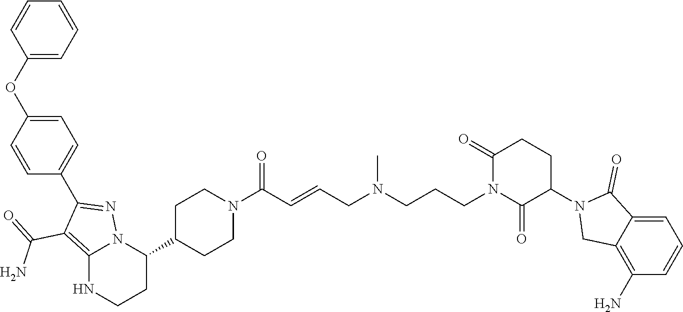 [SMILES: CN(C/C=C/C(=O)N1CCC([C@@H]2CCNc3c(C(N)=O)c(-c4ccc(Oc5ccccc5)cc4)nn32)CC1)CCCN1C(=O)CCC(N2Cc3c(N)cccc3C2=O)C1=O]

The titled compound was synthesized in the procedures similar to Example 53. ¹H NMR (400 MHz, DMSO) δ_{H} 7.50 (d, J=8.0 Hz, 2H), 7.42 (t, J=7.4 Hz, 2H), 7.18 (d, J=6.0 Hz, 2H), 7.07 (t, J=9.0 Hz, 4H), 6.91 (d, J=6.8 Hz, 1H), 6.80 (d, J=7.6 Hz, 1H), 6.68 (s, 1H), 6.59 (s, 1H), 5.45 (s, 1H), 5.16 (d, J=14.0 Hz, 1H), 4.45 (s, 1H), 4.28-3.92 (m, 4H), 3.78-3.58 (m, 2H), 3.29 (s, 2H), 3.12-2.88 (m, 4H), 2.85-2.62 (m, 2H), 2.33-2.29 (m, 4H), 2.17-1.83 (m, 6H), 1.69-1.55 (m, 5H), 1.30-1.10 (s, 7H); [M+H]⁺=813.9.

### Example 55: (7S)-7-(1-((E)-4-((2-(2-(2-(3-(4-amino-1-oxoisoindolin-2-yl)-2, 6-dioxopiperidin-1-yl)ethoxy)ethoxy)ethyl)(methyl)amino)but-2-enoyl)piperidin-4-yl)-2-(4-phenoxyphenyl)-4, 5, 6, 7-tetrahydropyrazolo[1,5-a]pyrimidine-3-carboxamide

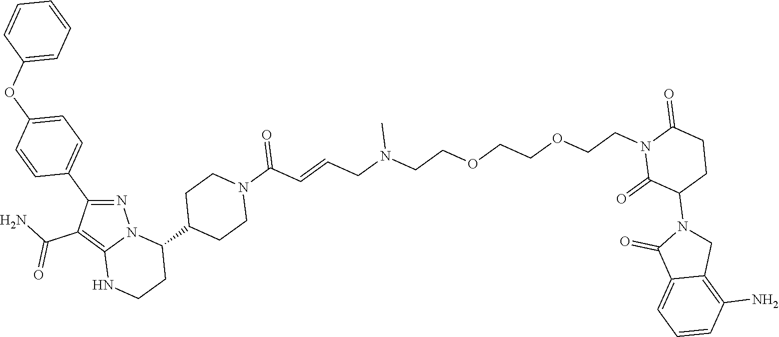 [SMILES: CN(C/C=C/C(=O)N1CCC([C@@H]2CCNc3c(C(N)=O)c(-c4ccc(Oc5ccccc5)cc4)nn32)CC1)CCOCCOCCN1C(=O)CCC(N2Cc3c(N)cccc3C2=O)C1=O]

The titled compound was synthesized in the procedures similar to Example 53. ¹H NMR (400 MHz, DMSO) δ_{H} 7.50 (d, J=8.0 Hz, 2H), 7.42 (s, 2H), 7.27-7.18 (m, 2H), 7.07 (t, J=8.4 Hz, 4H), 6.91 (d, J=6.8 Hz, 1H), 6.80 (d, J=8.4 Hz, 1H), 6.68 (s, 1H), 6.58 (s, 2H), 5.43 (s, 2H), 5.17 (s, 1H), 4.45-4.32 (m, 2H), 4.24-4.20 (m, 2H), 4.12-3.98 (m, 4H), 3.90-3.92 (m, 5H), 3.54-3.44 (m, 8H), 3.10-2.92 (m, 4H), 2.78-2.75 (m, 4H), 2.33-2.27 (m, 3H), 2.04-1.91 (m, 4H), 1.73-1.56 (m, 3H); [M+H]⁺=887.9.

### Example 56: (7S)-7-(1-(3-(4-(4-((3-(3-(4-amino-1-oxoisoindolin-2-yl)-2, 6-dioxopiperidin-1-yl)propanamido)methyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

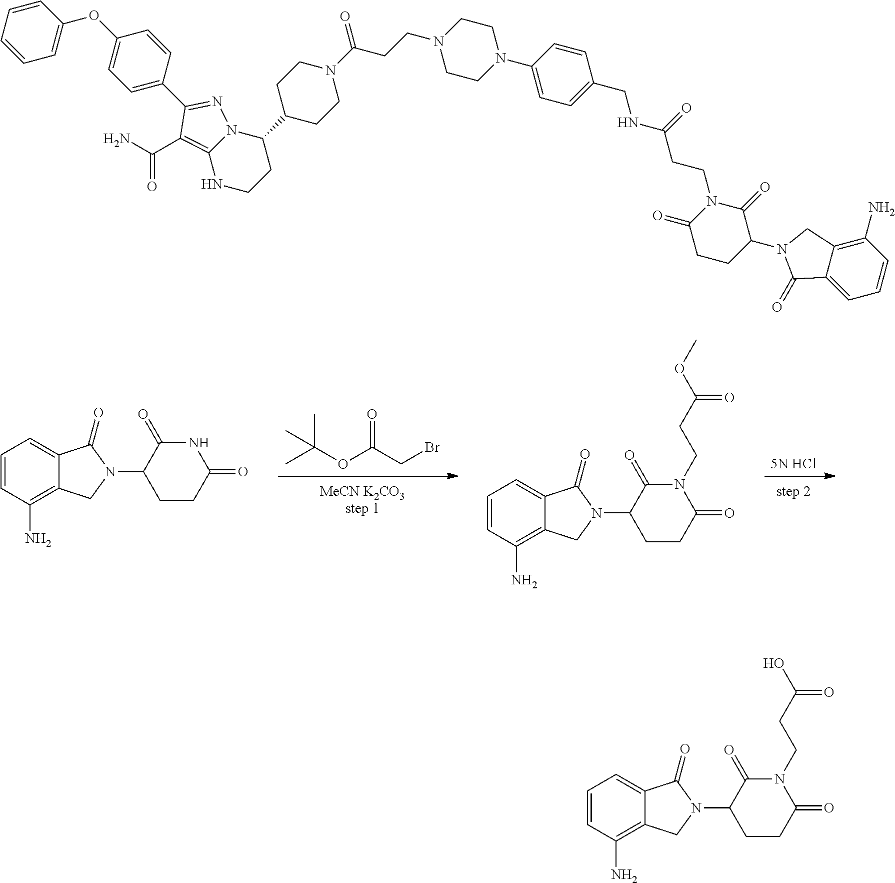

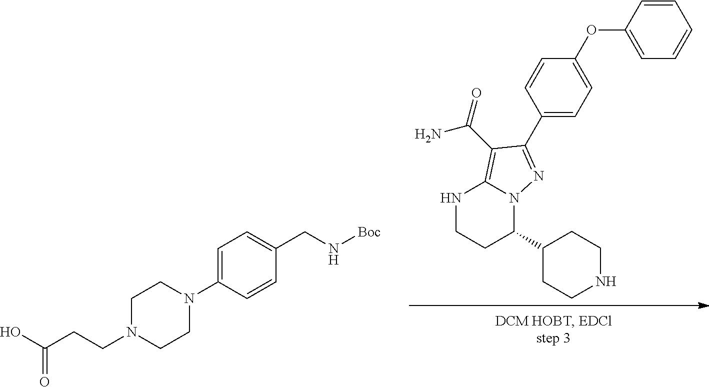 [SMILES: CC(C)(C)OC(=O)NCc1ccc(N2CCN(CCC(=O)O)CC2)cc1 ; NC(=O)c1c(-c2ccc(Oc3ccccc3)cc2)nn2c1NCC[C@H]2C1CCNCC1]

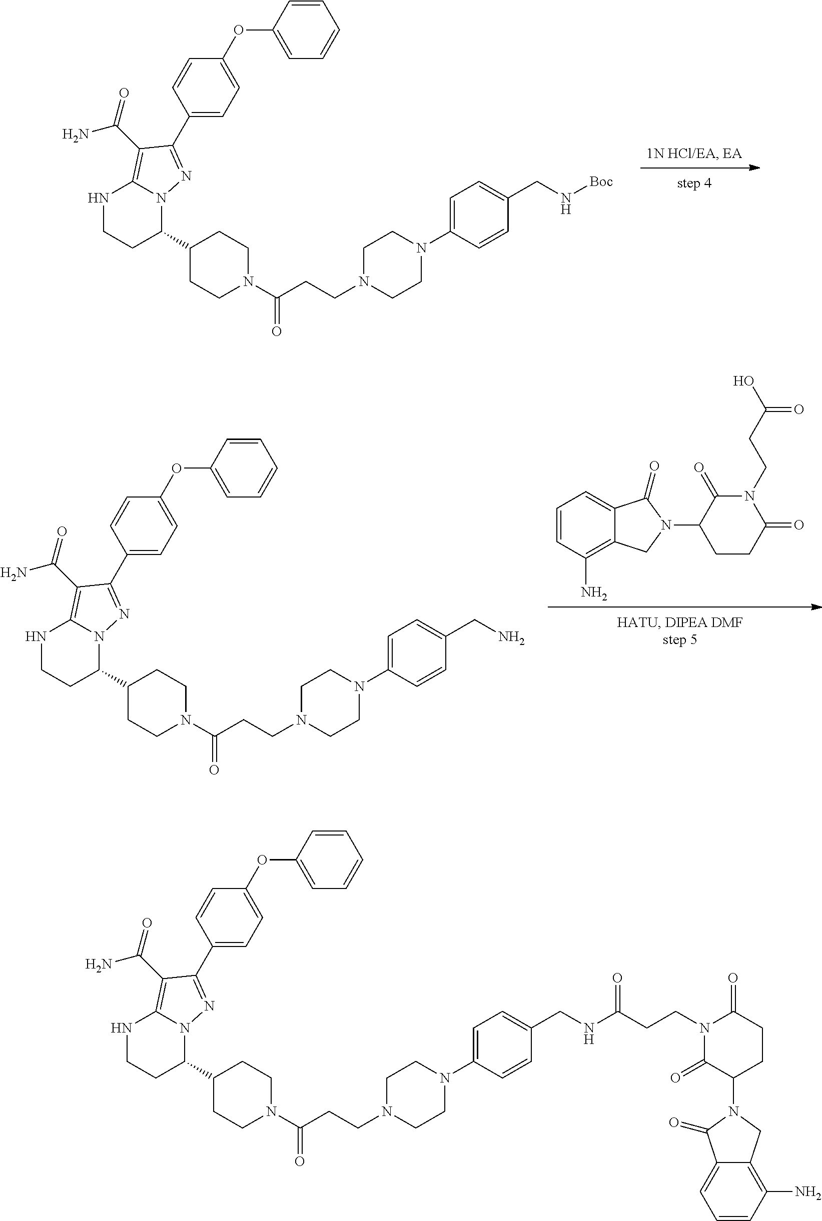 [SMILES: CC(C)(C)OC(=O)NCc1ccc(N2CCN(CCC(=O)N3CCC([C@@H]4CCNc5c(C(N)=O)c(-c6ccc(Oc7ccccc7)cc6)nn54)CC3)CC2)cc1 ; "1N HCl/EA, EA" ; NCc1ccc(N2CCN(CCC(=O)N3CCC([C@@H]4CCNc5c(C(N)=O)c(-c6ccc(Oc7ccccc7)cc6)nn54)CC3)CC2)cc1 ; Nc1cccc2c1CN(C1CCC(=O)N(CCC(=O)O)C1=O)C2=O ; NC(=O)c1c(-c2ccc(Oc3ccccc3)cc2)nn2c1NCC[C@H]2C1CCN(C(=O)CCN2CCN(c3ccc(CNC(=O)CCN4C(=O)CCC(N5Cc6c(N)cccc6C5=O)C4=O)cc3)CC2)CC1]

### Step 1: methyl 3-(3-(4-amino-1-oxoisoindolin-2-yl)-2,6-dioxopiperidin-1-yl)propanoate

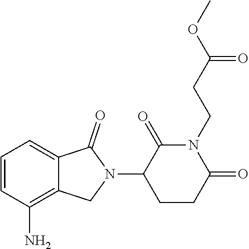 [SMILES: COC(=O)CCN1C(=O)CCC(N2Cc3c(N)cccc3C2=O)C1=O]

A mixture of 3-(4-amino-1-oxoisoindolin-2-yl)piperidine-2, 6-dione (0.5 g, 1.93 mmol), methyl tert-butyl 2-bromoacetate (0.54 g, 3.86 mmol) and K₂CO₃ (0.8 g, 5.79 mmol) in CH₃CN (20 mL) was stirred at 75° C. overnight. TLC (EA, Rf=0.2) showed the reaction was completed. Filtered and concentrated to give crude product, which was purified with silica gel chromatography (MeOH: DCM=1:30-1:10) to give the product (0.3 g, 60%). [M+H]⁺=346.0.

### Step 2: 3-(3-(4-amino-1-oxoisoindolin-2-yl)-2,6-dioxopiperidin-1-yl)propanoic acid

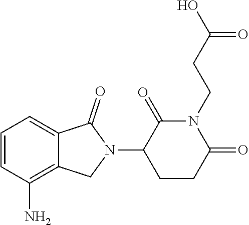 [SMILES: Nc1cccc2c1CN(C1CCC(=O)N(CCC(=O)O)C1=O)C2=O]

A mixture of methyl 3-(3-(4-amino-1-oxoisoindolin-2-yl)-2,6-dioxopiperidin-1-yl)propanoate (200 mg, 0.58 mmol) in 6N/HCl (10 mL) was stirred at R.T. for 2 hours. LCMS showed the reaction was completed. The solvent was removed in vacuo to give the product (150 mg, 70%), which was used for the next step without further purification. [M+H]⁺=332.0.

### Step 3: tert-butyl (S)-(4-(4-(3-(4-(3-carbamoyl-2-(4-phenoxyphenyl)-4, 5, 6, 7-tetrahydropyrazolo[1,5-a]pyrimidin-7-yl)piperidin-1-yl)-3-oxopropyl)piperazin-1-yl)benzyl)carbamate

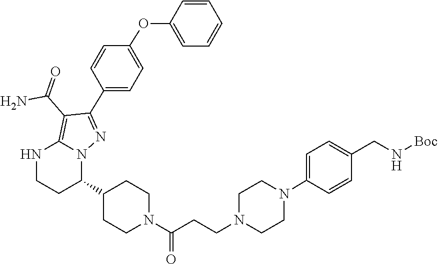 [SMILES: CC(C)(C)OC(=O)NCc1ccc(N2CCN(CCC(=O)N3CCC([C@@H]4CCNc5c(C(N)=O)c(-c6ccc(Oc7ccccc7)cc6)nn54)CC3)CC2)cc1]

A mixture of (S)-2-(4-phenoxyphenyl)-7-(piperidin-4-yl)-4, 5, 6, 7-tetrahydropyrazolo[1, 5-a]pyrimidine-3-carboxamide (1.6 g, 3.87 mmol), 3-(4-(4-(((tert-butoxycarbonyl)amino)methyl)phenyl)piperazin-1-yl)propanoic acid (1.3 g, 3.87 mmol), HOBT (1.05 g, 7.74 mmol) and EDCI (1.48 g, 7.74 mmol) in DCM (100 mL) was stirred at room temperature overnight. TLC (PE:EA=1:1, Rf=0.3) showed the reaction was completed. Then was added water 10 ml and extracted with DCM (10 ml*3). The combined organic layer was dried over Na₂SO₄. Filtered and concentrated to give crude product, which was purified with silica gel chromatography (MeOH: DCM=1:30-1:10) to give the product (2.6 g, 88%). [M+H]⁺=763.0.

### Step 4: (S)-7-(1-(3-(4-(4-(aminomethyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

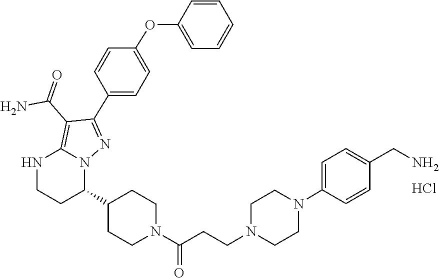 [SMILES: NCc1ccc(N2CCN(CCC(=O)N3CCC([C@@H]4CCNc5c(C(N)=O)c(-c6ccc(Oc7ccccc7)cc6)nn54)CC3)CC2)cc1 ; Cl]

A mixture of tert-butyl (S)-(4-(4-(3-(4-(3-carbamoyl-2-(4-phenoxyphenyl)-4, 5, 6, 7-tetrahydropyrazolo[1,5-a]pyrimidin-7-yl)piperidin-1-yl)-3-oxopropyl)piperazin-1-yl)benzyl)carbamate in 1N EA/HCl (30 mL) was stirred at RT for 2 hours. LCMS showed the reaction was completed. The solvent was removed in vacuo to give the product (2.3 g, 96.6%), which was used for the next step without further purification. [M+H]⁺=663.0.

### Step 5: (7S)-7-(1-(3-(4-(4-((3-(3-(4-amino-1-oxoisoindolin-2-yl)-2,6-dioxopiperidin-1-yl)propanamido)methyl)phenyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

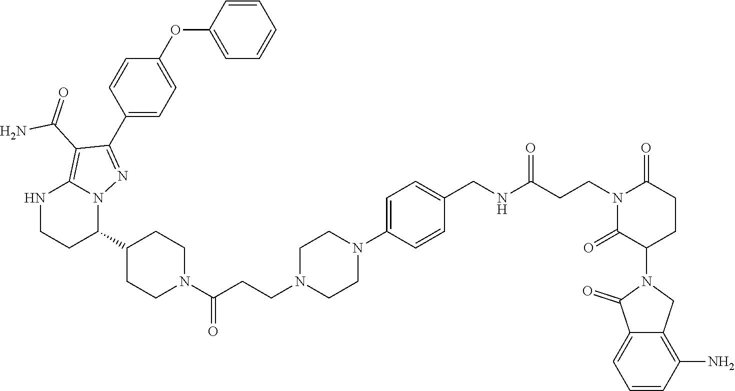 [SMILES: NC(=O)c1c(-c2ccc(Oc3ccccc3)cc2)nn2c1NCC[C@H]2C1CCN(C(=O)CCN2CCN(c3ccc(CNC(=O)CCN4C(=O)CCC(N5Cc6c(N)cccc6C5=O)C4=O)cc3)CC2)CC1]

A mixture of (S)-7-(1-(3-(4-(4-(aminomethyl)phenyl)piperazin-1-yl)propanoyl) piperidin-4-yl)-2-(4-phenoxyphenyl)-4, 5, 6, 7-tetrahydropyrazolo[1,5-a]pyrimidine-3-carboxamide (0.1 g, 0.302 mmol), 3-(3-(4-amino-1-oxoisoindolin-2-yl)-2,6-dioxopiperidin-1-yl)propanoic acid (130 mg, 0.39 mmol), HATU (137.8 mg, 0.362 mmol) in DMF (10 mL) was stirred at room temperature overnight. LCMS showed the reaction was completed. The solvent was evaporated under reduced pressure and the residue was purified with pre-HPLC to give the title product (10 mg, 20%). ¹H NMR (400 MHz, DMSO) δ_{H} 8.29 (t, J=5.6 Hz, 1H), 8.15 (s, 1H), 7.50 (d, J=8.4 Hz, 2H), 7.42 (t, J=7.8 Hz, 2H), 7.18 (q, J=7.6 Hz, 2H), 7.10-7.02 (m, 6H), 6.92 (d, J=7.6 Hz, 1H), 6.85 (d, J=8.4 Hz, 2H), 6.80 (d, J=8.0 Hz, 1H), 6.67 (s, 1H), 5.41 (s, 2H), 5.16 (dd, J=13.2, 4.8 Hz, 1H), 4.43 (s, 2H), 4.26-3.76 (m, 11H), 3.11-2.91 (m, 8H), 2.81-2.64 (m, 4H), 2.28 (m, 5H), 1.96 (m, 4H), 1.79-1.39 (m, 6H); [M+H]⁺=975.9.

### Example 57: (7S)-7-(1-(3-(4-(2-((2-(2, 6-dioxopiperidin-3-yl)-1, 3-dioxoisoindolin-5-yl)amino)acetamido)phenyl)propanoyl)piperidin-4-yl)-2-(4-phenoxyphenyl)-4, 5, 6, 7-tetrahydropyrazolo[1,5-a] pyrimidine-3-carboxamide

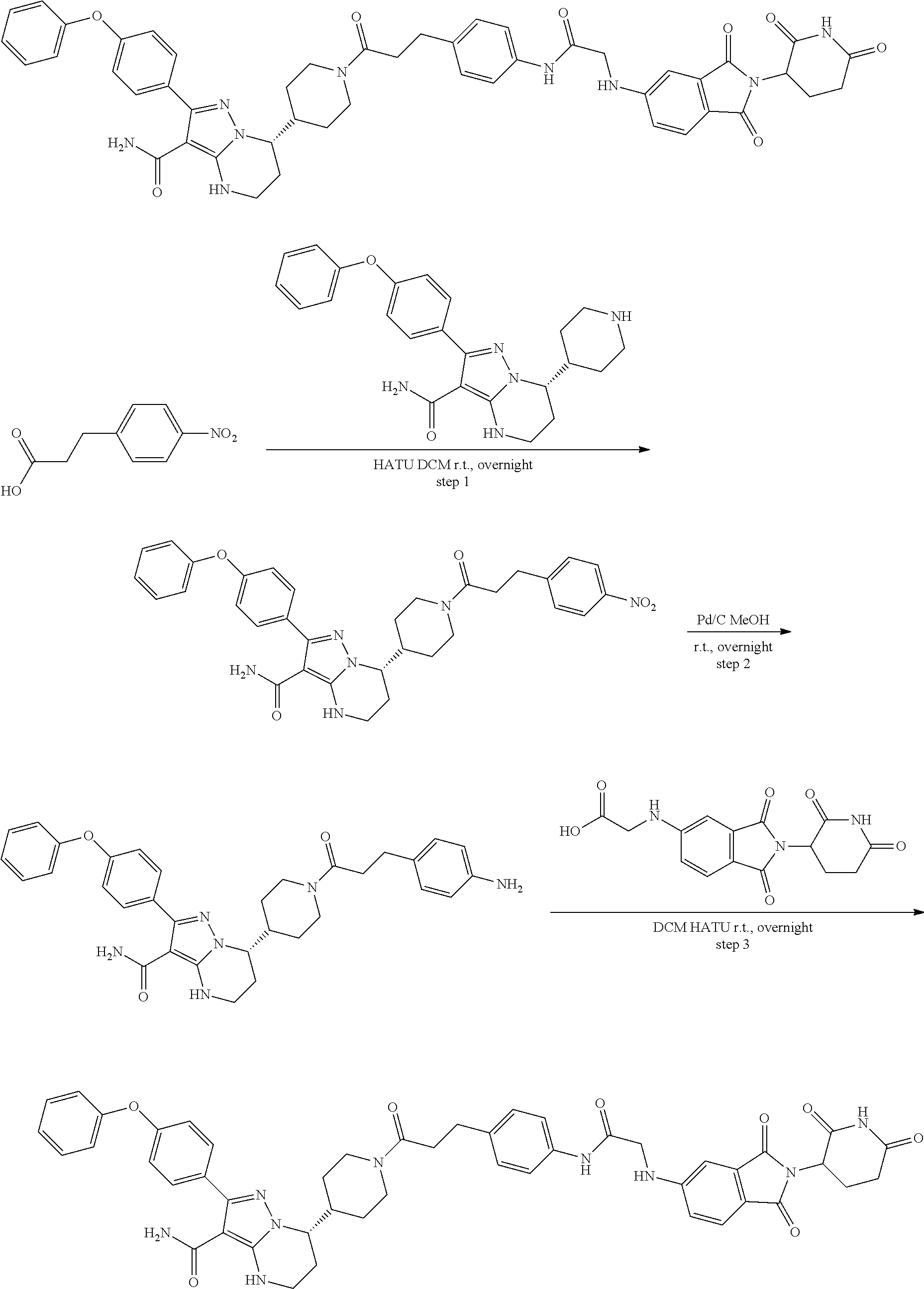

### Step 1: (S)-7-(1-(3-(4-nitrophenyl)propanoyl)piperidin-4-yl)-2-(4-phenoxyphenyl)-4, 5, 6, 7-tetrahydropyrazolo[1,5-a]pyrimidine-3-carboxamide

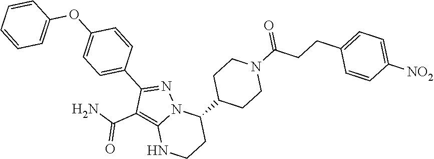 [SMILES: NC(=O)c1c(-c2ccc(Oc3ccccc3)cc2)nn2c1NCC[C@H]2C1CCN(C(=O)CCc2ccc([N+](=O)[O-])cc2)CC1]

A mixture of (S)-2-(4-phenoxyphenyl)-7-(piperidin-4-yl)-4, 5, 6, 7-tetrahydropyrazolo[1, 5-a]pyrimidine-3-carboxamide (417 mg, 1.0 mmol), 3-(4-nitrophenyl)propanoic acid (195 mg, 1.0 mmol), HATU (570 mg, 1.5 mmol) in DCM (10 mL) was stirred at room temperature overnight. After LCMS indicated that the reaction was completed, the mixture was concentrated in vacuo, and the residue was purified with silica gel column chromatography (PE:EA=1:1) to afford desired product (300 mg, 50.0%). [M+H]⁺=596.0.

### Step 2: (S)-7-(1-(3-(4-aminophenyl)propanoyl)piperidin-4-yl)-2-(4-phenoxyphenyl)-4, 5, 6, 7-tetrahydropyrazolo[1,5-a]pyrimidine-3-carboxamide

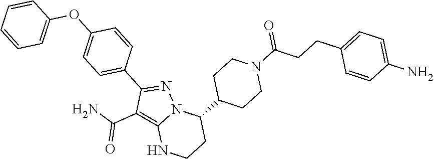 [SMILES: NC(=O)c1c(-c2ccc(Oc3ccccc3)cc2)nn2c1NCC[C@H]2C1CCN(C(=O)CCc2ccc(N)cc2)CC1]

To a solution of (S)-7-(1-(3-(4-nitrophenyl)propanoyl)piperidin-4-yl)-2-(4-phenoxyphenyl)-4, 5, 6, 7-tetrahydropyrazolo[1,5-a]pyrimidine-3-carboxamide (300 mg, 0.505 mmol) in MeOH (10 mL) was added Pd/C (5%, 30 mg). The mixture was stirred overnight under H₂ at R.T. After LCMS indicated that the reaction was completed, the solid was filtered off and the filtrate was concentrated. The residue was purified with pre-TLC (DCM:MeOH=20:1) to give the target product (50 mg, 26% yield). [M+H]⁺=565.0.

### Step 3: (7S)-7-(1-(3-(4-(2-((2-(2, 6-dioxopiperidin-3-yl)-1, 3-dioxoisoindolin-5-yl)amino)acetamido)phenyl)propanoyl)piperidin-4-yl)-2-(4-phenoxyphenyl)-4, 5, 6, 7-tetrahydropyrazolo[1,5-a]pyrimidine-3-carboxamide

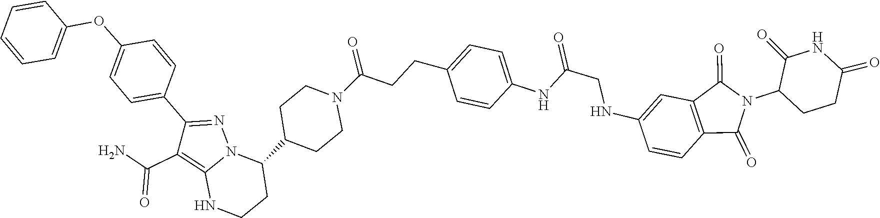 [SMILES: NC(=O)c1c(-c2ccc(Oc3ccccc3)cc2)nn2c1NCC[C@H]2C1CCN(C(=O)CCc2ccc(NC(=O)CNc3ccc4c(c3)C(=O)N(C3CCC(=O)NC3=O)C4=O)cc2)CC1]

A mixture of (S)-7-(1-(3-(4-aminophenyl)propanoyl)piperidin-4-yl)-2-(4-phenoxyphenyl)-4, 5, 6, 7-tetrahydropyrazolo[1,5-a]pyrimidine-3-carboxamide (50 mg, 0.0887 mmol), (2-(2, 6-dioxopiperidin-3-yl)-1, 3-dioxoisoindolin-5-yl)glycine (35.5 mg, 0.106 mmol) and HATU (54 mg, 0.142 mmol) in DCM (5 mL) was stirred at room temperature overnight. After LCMS indicated that the reaction was completed, the mixture was concentrated in vacuo, and the residue was purified with pre-TLC (DCM:MeOH=15:1) to afford the target product (17.44 mg, 23.3%). ¹H NMR (400 MHz, DMSO) δ_{H} 11.06 (s, 1H), 10.07 (s, 1H), 7.60 (d, J=8.0 Hz, 1H), 7.49 (d, J=8.0 Hz, 4H), 7.42 (t, J=8.0 Hz, 3H), 7.16 (d, J=4.0 Hz, 3H), 7.12-6.98 (m, 5H), 6.92 (d, J=8.0 Hz, 1H), 6.66 (s, 1H), 5.03 (dd, J=12.0, 4.0 Hz, 1H), 4.45 (s, 1H), 4.08 (d, J=4.0 Hz, 2H), 3.98 (dd, J=12.0, 4.0 Hz, 1H), 3.90 (t, J=12.0 Hz, 1H), 3.28 (s, 2H), 2.96-2.81 (m, 2H), 2.74 (t, J=8.0 Hz, 2H), 2.60-2.53 (m, 3H), 2.48-2.37 (m, 2H), 2.22 (s, 1H), 2.05-1.80 (m, 3H), 1.65 (d, J=12.0 Hz, 1H), 1.46 (dd, J=32.0, 12.0 Hz, 1H), 1.26-0.96 (m, 2H); [M+H]⁺=878.0.

### Example 58: (7S)-7-(1-(4-(4-(2-((2-(2, 6-dioxopiperidin-3-yl)-1, 3-dioxoisoindolin-5-yl)amino)acetamido)phenyl)butanoyl)piperidin-4-yl)-2-(4-phenoxyphenyl)-4, 5, 6, 7-tetrahydropyrazolo[1,5-a] pyrimidine-3-carboxamide

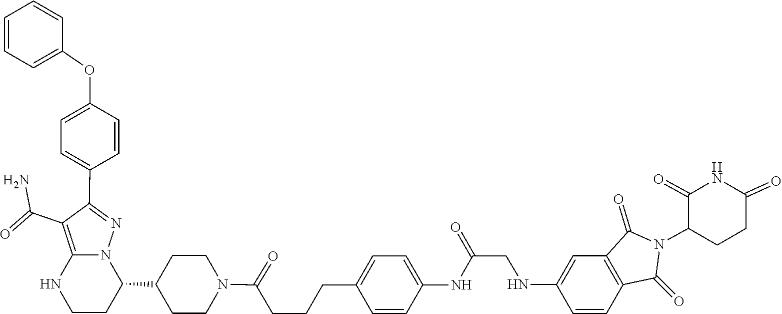 [SMILES: NC(=O)c1c(-c2ccc(Oc3ccccc3)cc2)nn2c1NCC[C@H]2C1CCN(C(=O)CCCc2ccc(NC(=O)CNc3ccc4c(c3)C(=O)N(C3CCC(=O)NC3=O)C4=O)cc2)CC1]

The titled compound was synthesized in the procedures similar to Example 57. ¹H NMR (400 MHz, DMSO) δ_{H} 11.06 (s, 1H), 10.07 (s, 1H), 7.60 (d, J=8.0 Hz, 1H), 7.50 (d, J=8.4 Hz, 4H), 7.46-7.37 (m, 3H), 7.21-6.97 (m, 8H), 6.92 (d, J=8.0 Hz, 1H), 6.67 (s, 1H), 5.03 (dd, J=12.0, 4.0 Hz, 1H), 4.45 (d, J=12.0 Hz, 1H), 4.07 (d, J=4.0 Hz, 2H), 4.05-3.96 (m, 1H), 3.84 (t, J=12.0 Hz, 1H), 3.31-3.26 (m, 2H), 2.99-2.81 (m, 2H), 2.62-2.52 (m, 4H), 2.47-2.39 (m, 1H), 2.31-2.16 (m, 3H), 2.05-1.86 (m, 3H), 1.80-1.63 (m, 3H), 1.51 (d, J=12.0 Hz, 1H), 1.34-1.07 (m, 2H); [M+H]⁺=892.0.

### Example 59: (7S)-7-(1-(5-(4-(2-((2-(2, 6-dioxopiperidin-3-yl)-1, 3-dioxoisoindolin-5-yl)amino)acetamido)phenyl)pentanoyl)piperidin-4-yl)-2-(4-phenoxyphenyl)-4, 5, 6, 7-tetrahydropyrazolo[1,5-a]pyrimidine-3-carboxamide

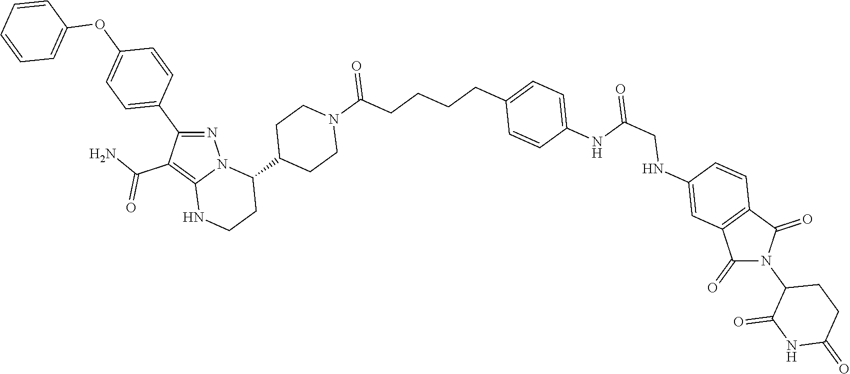 [SMILES: NC(=O)c1c(-c2ccc(Oc3ccccc3)cc2)nn2c1NCC[C@H]2C1CCN(C(=O)CCCCc2ccc(NC(=O)CNc3ccc4c(c3)C(=O)N(C3CCC(=O)NC3=O)C4=O)cc2)CC1]

### Example 60: (7S)-7-(1-(3-((1r, 4S)-4-(2-((2-(2, 6-dioxopiperidin-3-yl)-1, 3-dioxoisoindolin-5-yl)amino)acetamido)cyclohexyl)propanoyl)piperidin-4-yl)-2-(4-phenoxyphenyl)-4, 5, 6, 7-tetrahydropyrazolo[1,5-a]pyrimidine-3-carboxamide

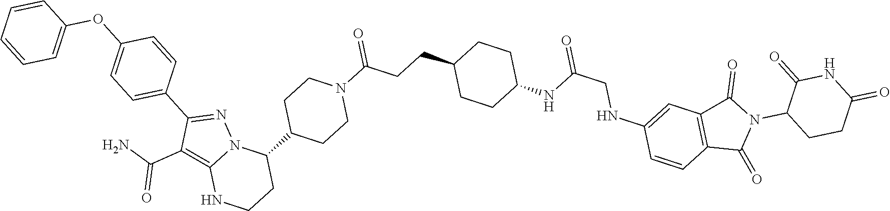 [SMILES: NC(=O)c1c(-c2ccc(Oc3ccccc3)cc2)nn2c1NCC[C@H]2C1CCN(C(=O)CC[C@H]2CC[C@H](NC(=O)CNc3ccc4c(c3)C(=O)N(C3CCC(=O)NC3=O)C4=O)CC2)CC1]

### Example 61: (7S)-7-(1-(4-((1r, 4R)-4-(2-((2-(2, 6-dioxopiperidin-3-yl)-1, 3-dioxoisoindolin-5-yl)amino)acetamido)cyclohexyl)butanoyl)piperidin-4-yl)-2-(4-phenoxyphenyl)-4, 5, 6, 7-tetrahydropyrazolo[1,5-a]pyrimidine-3-carboxamide

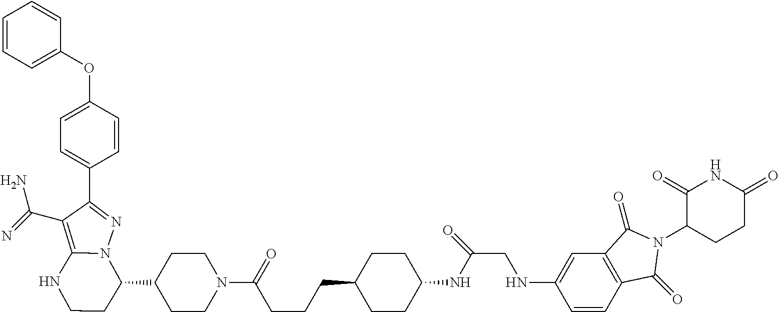 [SMILES: N=C(N)c1c(-c2ccc(Oc3ccccc3)cc2)nn2c1NCC[C@H]2C1CCN(C(=O)CCC[C@H]2CC[C@H](NC(=O)CNc3ccc4c(c3)C(=O)N(C3CCC(=O)NC3=O)C4=O)CC2)CC1]

### Example 62: (7S)-7-(1-(5-((1r, 4R)-4-(2-((2-(2, 6-dioxopiperidin-3-yl)-1, 3-dioxoisoindolin-5-yl)amino)acetamido)cyclohexyl)pentanoyl)piperidin-4-yl)-2-(4-phenoxyphenyl)-4, 5, 6, 7-tetrahydropyrazolo[1,5-a]pyrimidine-3-carboxamide

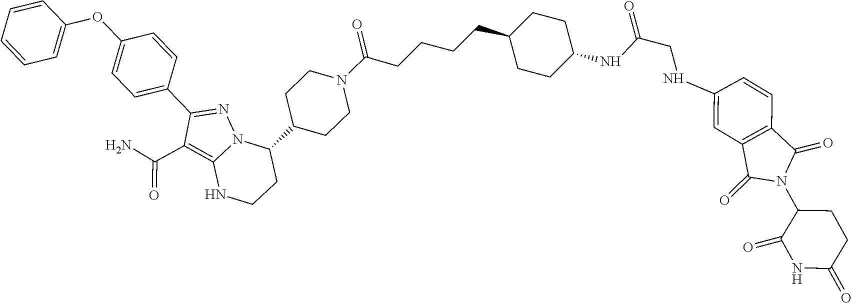 [SMILES: NC(=O)c1c(-c2ccc(Oc3ccccc3)cc2)nn2c1NCC[C@H]2C1CCN(C(=O)CCCC[C@H]2CC[C@H](NC(=O)CNc3ccc4c(c3)C(=O)N(C3CCC(=O)NC3=O)C4=O)CC2)CC1]

### Example 63: (7S)-7-(1-(3-(4-((2-(2, 6-dioxopiperidin-3-yl)-1, 3-dioxoisoindolin-5-yl)glycyl)piperazin-1-yl)propanoyl)piperidin-4-yl)-2-(4-phenoxyphenyl)-4, 5, 6, 7-tetrahydropyrazolo[1,5-a]pyrimidine-3-carboxamide

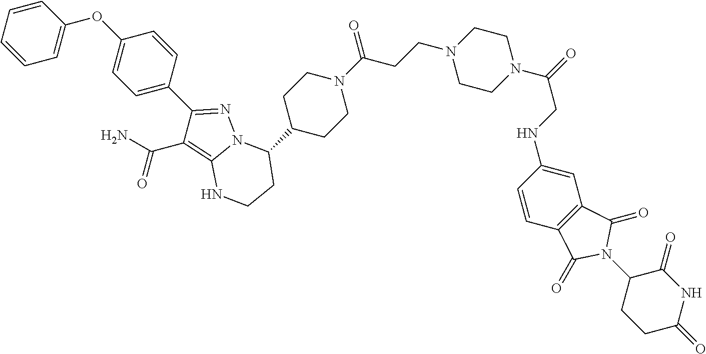 [SMILES: NC(=O)c1c(-c2ccc(Oc3ccccc3)cc2)nn2c1NCC[C@H]2C1CCN(C(=O)CCN2CCN(C(=O)CNc3ccc4c(c3)C(=O)N(C3CCC(=O)NC3=O)C4=O)CC2)CC1]

The titled compound was synthesized in the procedures similar to Example 53. ¹H NMR (400 MHz, DMSO) δ_{H} 11.07 (s, 1H), 7.57 (d, J=8.0 Hz, 1H), 7.50 (d, J=8.0 Hz, 2H), 7.43-7.40 (m, 2H), 7.19-7.00 (m, 8H), 6.68 (s, 1H), 5.06-5.02 (m, 1H), 4.46-4.43 (m, 1H), 4.12-4.11 (m, 2H), 4.01-3.95 (m, 2H), 3.53-3.45 (m, 4H), 3.31-3.27 (m, 2H), 2.99-2.83 (m, 3H), 2.59-2.55 (m, 4H), 2.45-2.33 (m, 5H), 2.30-2.15 (m, 1H), 2.10-1.85 (m, 4H), 1.74-1.65 (m, 1H), 1.58-1.52 (m, 1H), 1.34-1.12 (m, 2H); [M+H]⁺=870.9.

### Example 64: (7S)-7-(1-(3-(((1-(2-(2, 6-dioxopiperidin-3-yl)-1, 3-dioxoisoindolin-5-yl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

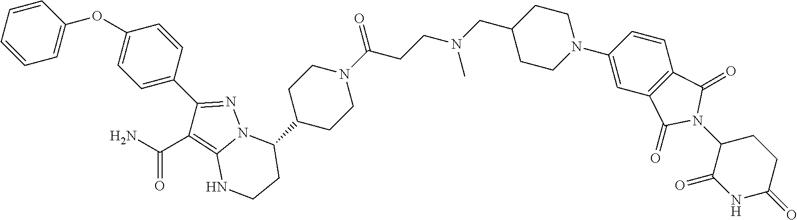 [SMILES: CN(CCC(=O)N1CCC([C@@H]2CCNc3c(C(N)=O)c(-c4ccc(Oc5ccccc5)cc4)nn32)CC1)CC1CCN(c2ccc3c(c2)C(=O)N(C2CCC(=O)NC2=O)C3=O)CC1]

The titled compound was synthesized in the procedures similar to Example 151. ¹H NMR (400 MHz, DMSO) δ_{H} 11.08 (s, 1H), 7.65-7.63 (m, 1H), 7.50 (d, J=8.0 Hz, 2H), 7.43-7.39 (m, 2H), 7.30 (s, 1H), 7.23-7.15 (m, 2H), 7.08-7.04 (m, 4H), 6.68 (s, 1H), 5.08-5.04 (m, 1H), 4.46-4.43 (m, 1H), 4.01-3.93 (m, 4H), 3.31-3.30 (m, 2H), 2.98-2.83 (m, 4H), 2.67-2.44 (m, 7H), 2.30-2.15 (m, 4H), 1.99-1.89 (m, 4H), 1.78-1.65 (m, 4H), 1.56-1.53 (m, 1H), 1.31-1.14 (m, 5H); [M+H]⁺=856.0.

### Example 65: (7S)-7-(1-(5-(4-((2-(2, 6-dioxopiperidin-3-yl)-1, 3-dioxoisoindolin-5-yl)glycyl)piperazin-1-yl)pentanoyl)piperidin-4-yl)-2-(4-phenoxyphenyl)-4, 5, 6, 7-tetrahydropyrazolo[1,5-a]pyrimidine-3-carboxamide

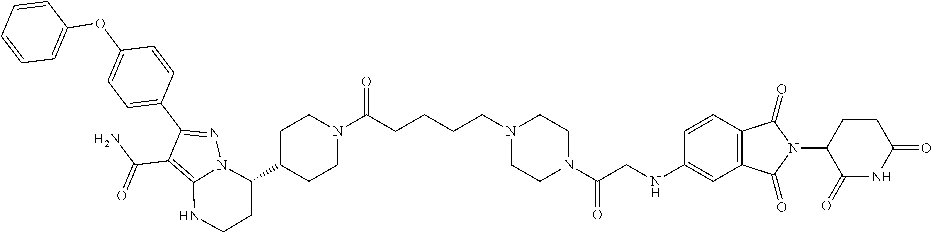 [SMILES: NC(=O)c1c(-c2ccc(Oc3ccccc3)cc2)nn2c1NCC[C@H]2C1CCN(C(=O)CCCCN2CCN(C(=O)CNc3ccc4c(c3)C(=O)N(C3CCC(=O)NC3=O)C4=O)CC2)CC1]

### Example 66: (7S)-7-(1-(3-((2-(2-((2-(2, 6-dioxopiperidin-3-yl)-1, 3-dioxoisoindolin-5-yl)amino)ethoxy)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

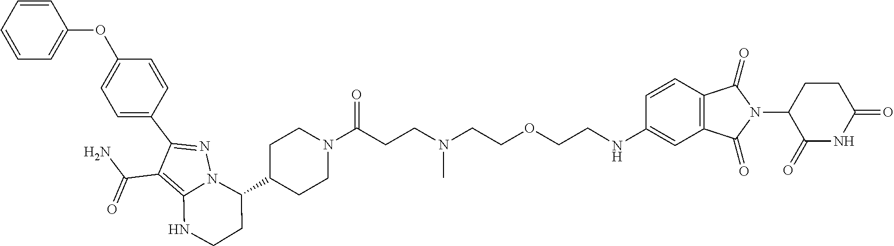 [SMILES: CN(CCOCCNc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

### Example 67: (7S)-7-(1-(3-(((4-((2-(2, 6-dioxopiperidin-3-yl)-1, 3-dioxoisoindolin-5-yl)amino)cyclohexyl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

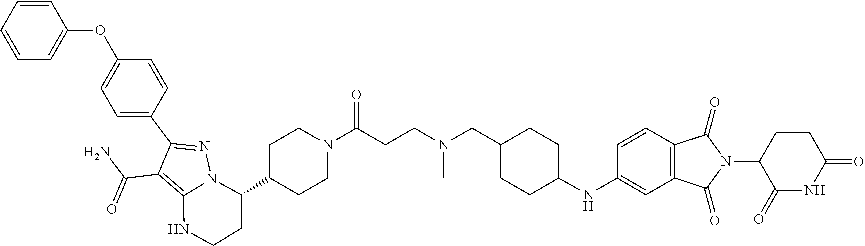 [SMILES: CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1]

### Example 68: (7S)-7-(1-(3-(((1-(2-(2, 6-dioxopiperidin-3-yl)-1, 3-dioxoisoindolin-5-yl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

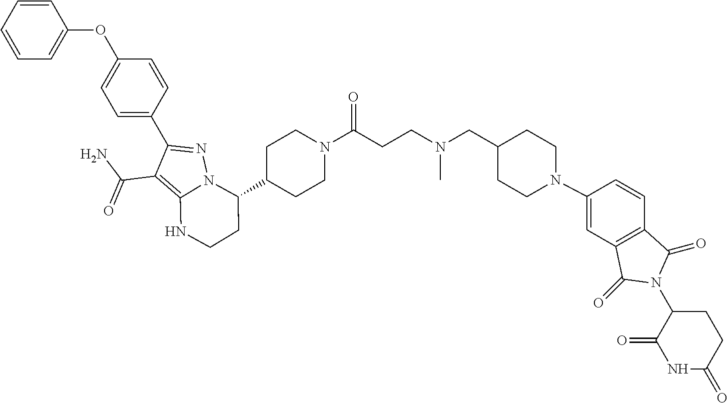 [SMILES: CN(CCC(=O)N1CCC([C@@H]2CCNc3c(C(N)=O)c(-c4ccc(Oc5ccccc5)cc4)nn32)CC1)CC1CCN(c2ccc3c(c2)C(=O)N(C2CCC(=O)NC2=O)C3=O)CC1]

### Example 69: (7S)-7-(1-(3-((4-((4-(2-(2, 6-dioxopiperidin-3-yl)-1, 3-dioxoisoindolin-5-yl)piperazin-1-yl)methyl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

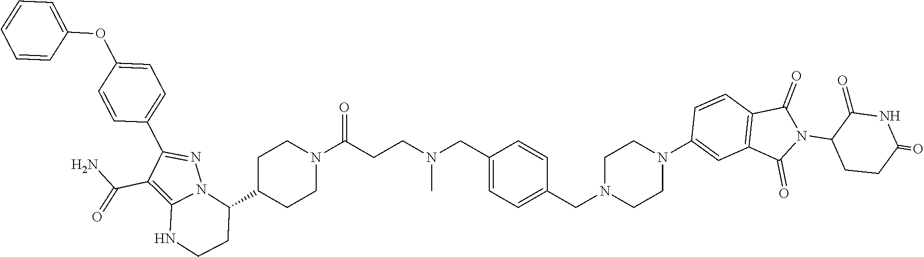 [SMILES: CN(CCC(=O)N1CCC([C@@H]2CCNc3c(C(N)=O)c(-c4ccc(Oc5ccccc5)cc4)nn32)CC1)Cc1ccc(CN2CCN(c3ccc4c(c3)C(=O)N(C3CCC(=O)NC3=O)C4=O)CC2)cc1]

### Example 70: (7S)-7-(1-(3-((4-(4-(2-(2, 6-dioxopiperidin-3-yl)-1, 3-dioxoisoindolin-5-yl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

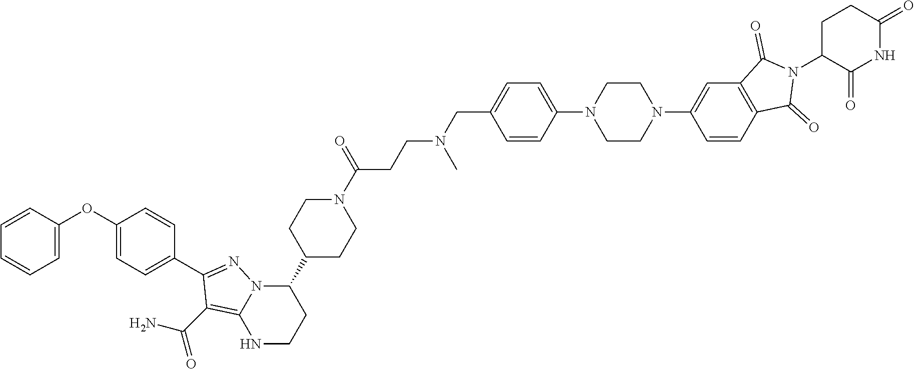 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)N(C3CCC(=O)NC3=O)C4=O)CC2)cc1]

### Example 71: (S)-7-(1-(3-((4-(4-(4-(2, 4-dioxotetrahydropyrimidin-1(2H)-yl)phenyl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

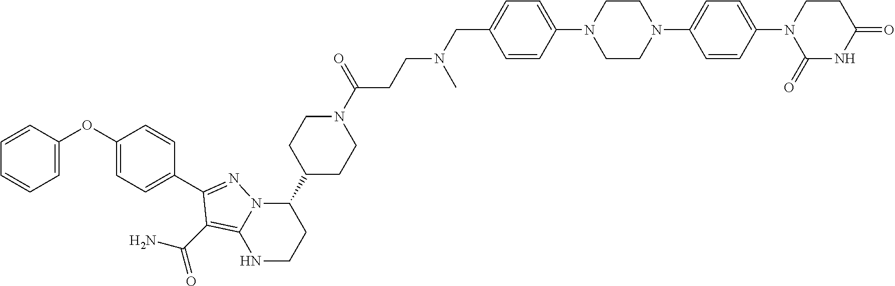 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc(N4CCC(=O)NC4=O)cc3)CC2)cc1]

### Example 72: (7S)-7-(1-(3-((2-(4-(4-((2, 6-dioxopiperidin-3-yl)amino)phenyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

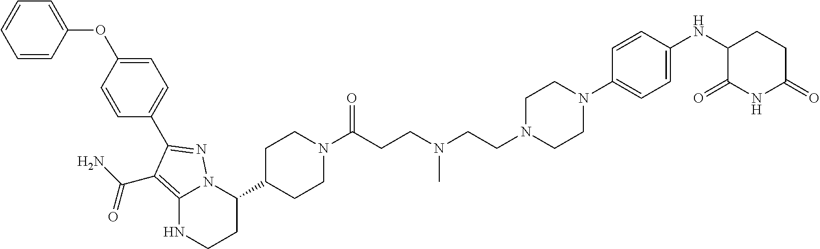 [SMILES: CN(CCC(=O)N1CCC([C@@H]2CCNc3c(C(N)=O)c(-c4ccc(Oc5ccccc5)cc4)nn32)CC1)CCN1CCN(c2ccc(NC3CCC(=O)NC3=O)cc2)CC1]

### Example 73: (S)-7-(1-(3-(((1-(4-(2, 4-dioxotetrahydropyrimidin-1(2H)-yl)phenyl)piperidin-4-yl)methyl)(methyl)amino)propanoyl)piperidin-4-yl)-2-(4-phenoxyphenyl)-4, 5, 6, 7-tetrahydropyrazolo[1, 5-a]pyrimidine-3-carboxamide

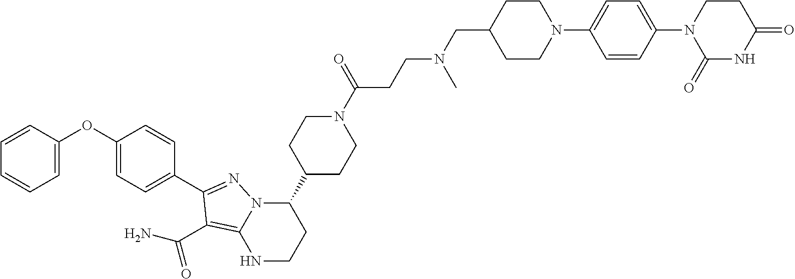 [SMILES: CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1]

### Example 74: (S)-7-(1-(3-(((1-(4-(2, 4-dioxotetrahydropyrimidin-1(2H)-yl)-2-fluorophenyl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

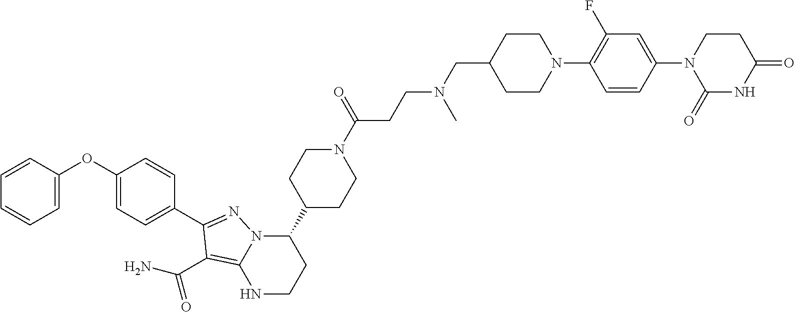 [SMILES: CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2F)CC1]

### Example 75: (S)-7-(1-(3-(((1-(6-(2, 4-dioxotetrahydropyrimidin-1(2H)-yl)-4-fluoropyridin-3-yl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

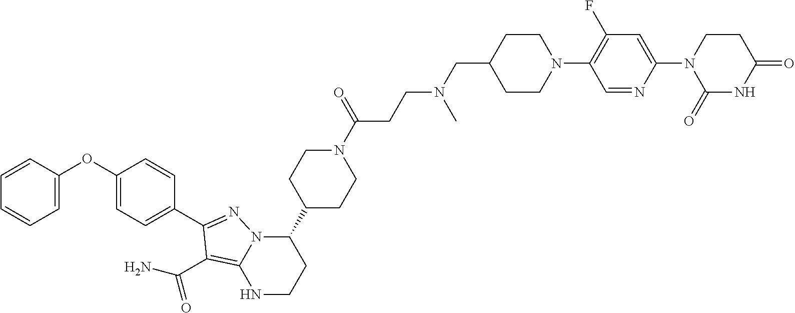 [SMILES: CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cc2F)CC1]

### Example 76: (S)-7-(1-(3-(((1-(5-(2, 4-dioxotetrahydropyrimidin-1(2H)-yl)pyrazin-2-yl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

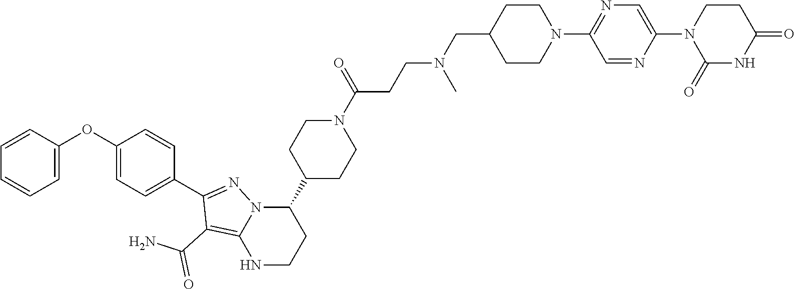 [SMILES: CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cn2)CC1]

### Example 77: (S)-7-(1-(3-(((1-(5-(2, 4-dioxotetrahydropyrimidin-1(2H)-yl)pyrimidin-2-yl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

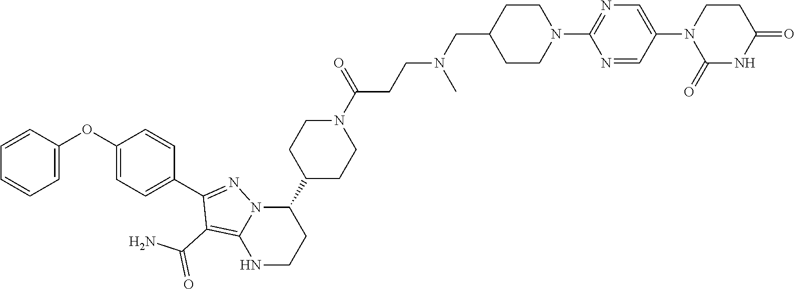 [SMILES: CN(CCC(=O)N1CCC([C@@H]2CCNc3c(C(N)=O)c(-c4ccc(Oc5ccccc5)cc4)nn32)CC1)CC1CCN(c2ncc(N3CCC(=O)NC3=O)cn2)CC1]

### Example 78: (S)-7-(1-(3-(7-(4-(2, 4-dioxotetrahydropyrimidin-1(2H)-yl)phenyl)-2, 7-diazaspiro[3.5]nonan-2-yl)propanoyl)piperidin-4-yl)-2-(4-phenoxyphenyl)-4, 5, 6, 7-tetrahydropyrazolo[1,5-a]pyrimidine-3-carboxamide

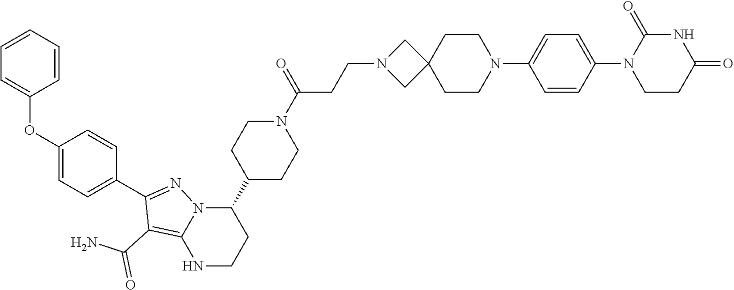 [SMILES: NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1]

### Example 79: (S, E)-7-(1-(3-(((1-(4-(2, 4-dioxotetrahydropyrimidin-1(2H)-yl)phenyl)piperidin-4-yl)methyl)(methyl)amino)acryloyl)piperidin-4-yl)-2-(4-phenoxyphenyl)-4, 5, 6, 7-tetrahydropyrazolo[1,5-a]pyrimidine-3-carboxamide

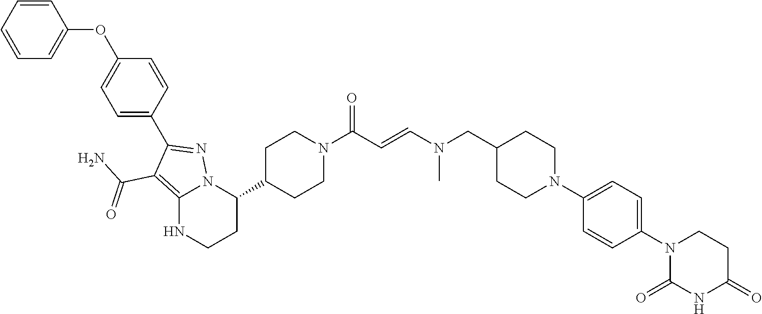 [SMILES: CN(/C=C/C(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1]

### Example 80: (S, E)-7-(1-(3-((2-(2-((4-(2, 4-dioxotetrahydropyrimidin-1(2H)- yl)phenyl)amino)ethoxy)ethyl)(methyl)amino)acryloyl)piperidin-4-yl)-2-(4-phenoxyphenyl)-4, 5, 6, 7-tetrahydropyrazolo[1,5-a]pyrimidine-3-carboxamide

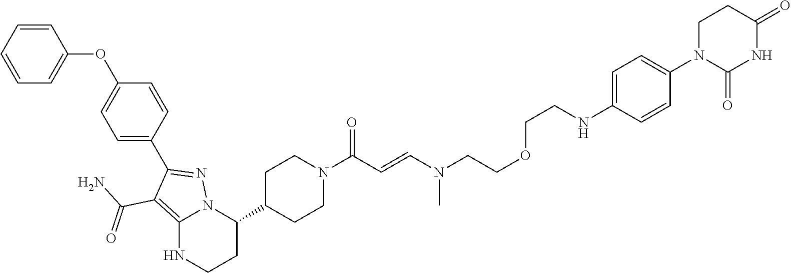 [SMILES: CN(/C=C/C(=O)N1CCC([C@@H]2CCNc3c(C(N)=O)c(-c4ccc(Oc5ccccc5)cc4)nn32)CC1)CCOCCNc1ccc(N2CCC(=O)NC2=O)cc1]

### Example 81: (S)-7-(1-(3-((2-(4-(3-(2, 4-dioxotetrahydropyrimidin-1(2H)-yl)benzoyl)piperidin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

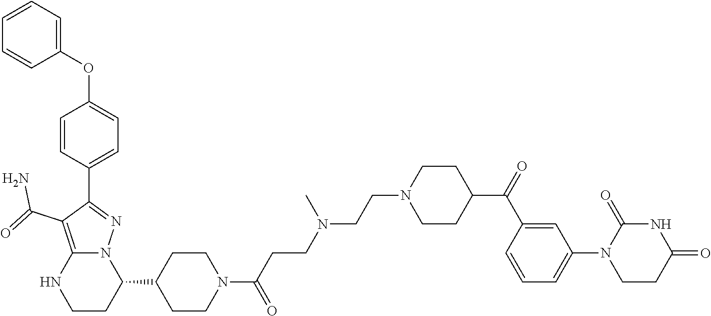 [SMILES: CN(CCC(=O)N1CCC([C@@H]2CCNc3c(C(N)=O)c(-c4ccc(Oc5ccccc5)cc4)nn32)CC1)CCN1CCC(C(=O)c2cccc(N3CCC(=O)NC3=O)c2)CC1]

### Example 82: (7S)-7-(1-((E)-3-((2-(2-((2-(2, 6-dioxopiperidin-3-yl)-1, 3-dioxoisoindolin-4-yl)oxy)acetamido)ethyl)(methyl)amino)acryloyl)piperidin-4-yl)-2-(4-phenoxyphenyl)-4, 5, 6, 7-tetrahydropyrazolo[1,5-a]pyrimidine-3-carboxamide

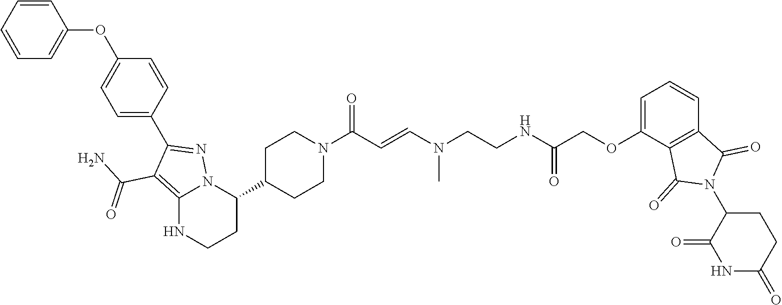 [SMILES: CN(/C=C/C(=O)N1CCC([C@@H]2CCNc3c(C(N)=O)c(-c4ccc(Oc5ccccc5)cc4)nn32)CC1)CCNC(=O)COc1cccc2c1C(=O)N(C1CCC(=O)NC1=O)C2=O]

### Example 83: (S)-7-(1-(3-(((4-(4-(4-(2, 4-dioxotetrahydropyrimidin-1(2H)-yl)phenyl)piperazin-1-yl)cyclohexyl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

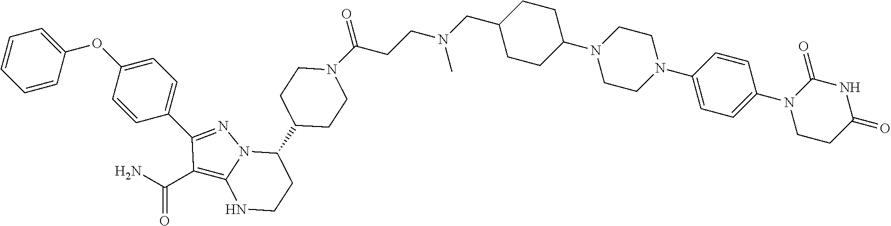 [SMILES: CN(CCC(=O)N1CCC([C@@H]2CCNc3c(C(N)=O)c(-c4ccc(Oc5ccccc5)cc4)nn32)CC1)CC1CCC(N2CCN(c3ccc(N4CCC(=O)NC4=O)cc3)CC2)CC1]

### Example 84: (S)-7-(1-(3-((2-(4-(4-(2, 4-dioxotetrahydropyrimidin-1(2H)-yl)phenyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

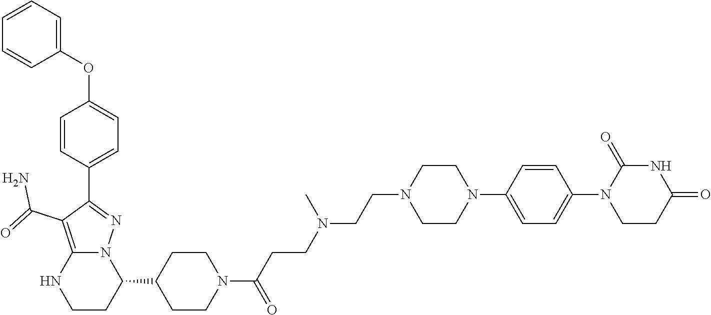 [SMILES: CN(CCC(=O)N1CCC([C@@H]2CCNc3c(C(N)=O)c(-c4ccc(Oc5ccccc5)cc4)nn32)CC1)CCN1CCN(c2ccc(N3CCC(=O)NC3=O)cc2)CC1]

### Example 85: (S)-7-(1-(3-((2-(4-(4-(2, 4-dioxotetrahydropyrimidin-1(2H)-yl)phenyl)piperazin-1-yl)-2-oxoethyl)(methyl)amino)propanoyl)piperidin-4-yl)-2-(4-phenoxyphenyl)-4, 5, 6, 7-tetrahydropyrazolo[1,5-a]pyrimidine-3-carboxamide

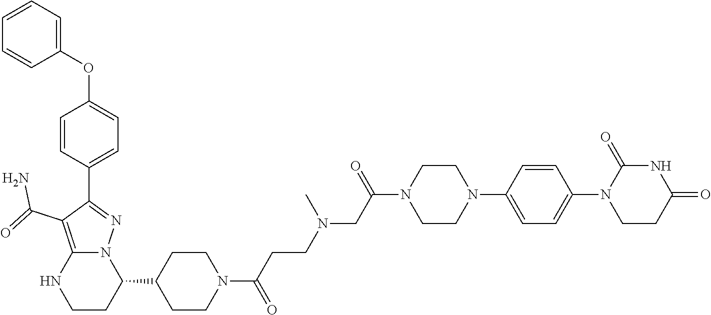 [SMILES: CN(CCC(=O)N1CCC([C@@H]2CCNc3c(C(N)=O)c(-c4ccc(Oc5ccccc5)cc4)nn32)CC1)CC(=O)N1CCN(c2ccc(N3CCC(=O)NC3=O)cc2)CC1]

### Example 86: (7S)-7-(1-(3-((2-((2-((2-(2, 6-dioxopiperidin-3-yl)-1, 3-dioxoisoindolin-4-yl)amino)ethyl)amino)-2-oxoethyl)(methyl)amino)propanoyl)piperidin-4-yl)-2-(4-phenoxyphenyl)-4, 5, 6, 7-tetrahydropyrazolo[1,5-a]pyrimidine-3-carboxamide

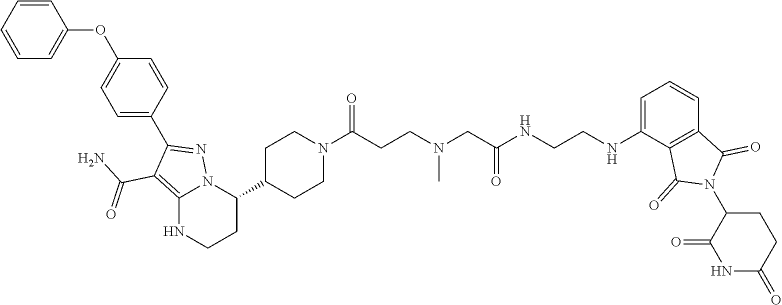 [SMILES: CN(CCC(=O)N1CCC([C@@H]2CCNc3c(C(N)=O)c(-c4ccc(Oc5ccccc5)cc4)nn32)CC1)CC(=O)NCCNc1cccc2c1C(=O)N(C1CCC(=O)NC1=O)C2=O]

### Example 87: (7S)-7-(1-(3-((2-(2-((2-(2, 6-dioxopiperidin-3-yl)-1, 3-dioxoisoindolin-4-yl)oxy)ethoxy)ethyl)(methyl)amino)propanoyl)piperidin-4-yl)-2-(4-phenoxyphenyl)-4, 5, 6, 7-tetrahydropyrazolo[1,5-a] pyrimidine-3-carboxamide

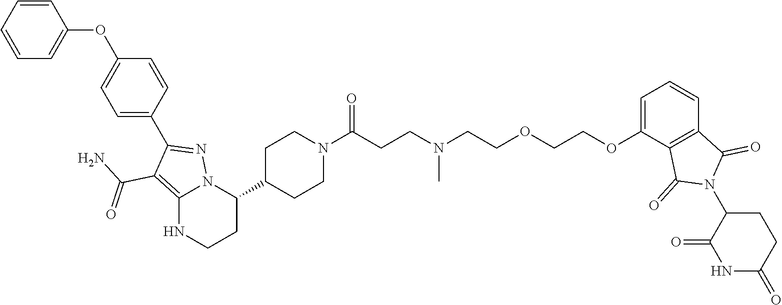 [SMILES: CN(CCOCCOc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

### Example 88: (7S)-7-(1-(3-((2-((2-((2-(2, 6-dioxopiperidin-3-yl)-1, 3-dioxoisoindolin-4-yl)oxy)ethyl)amino)-2-oxoethyl)(methyl)amino)propanoyl)piperidin-4-yl)-2-(4-phenoxyphenyl)-4, 5, 6, 7-tetrahydropyrazolo[1,5-a]pyrimidine-3-carboxamide

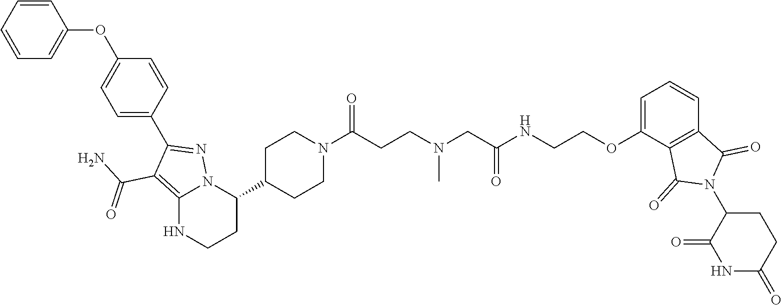 [SMILES: CN(CCC(=O)N1CCC([C@@H]2CCNc3c(C(N)=O)c(-c4ccc(Oc5ccccc5)cc4)nn32)CC1)CC(=O)NCCOc1cccc2c1C(=O)N(C1CCC(=O)NC1=O)C2=O]

### Example 89: (7S)-7-(1-(1-(2-(2-((2-(2, 6-dioxopiperidin-3-yl)-1, 3-dioxoisoindolin-4-yl)amino)ethoxy)ethyl)piperidine-3-carbonyl)piperidin-4-yl)-2-(4-phenoxyphenyl)-4, 5, 6, 7-tetrahydropyrazolo[1, 5-a]pyrimidine-3-carboxamide

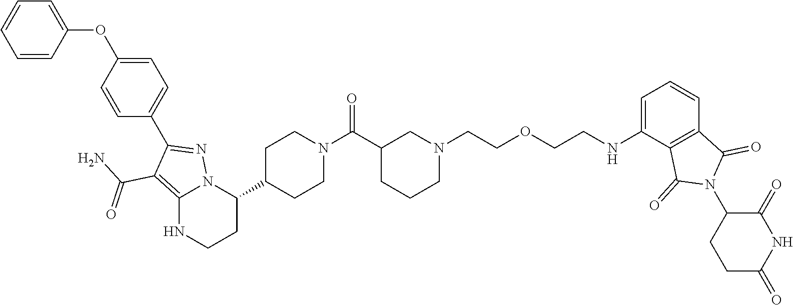 [SMILES: NC(=O)c1c(-c2ccc(Oc3ccccc3)cc2)nn2c1NCC[C@H]2C1CCN(C(=O)C2CCCN(CCOCCNc3cccc4c3C(=O)N(C3CCC(=O)NC3=O)C4=O)C2)CC1]

### Example 90: (7S)-7-(1-(3-(3-(2-((2-(2, 6-dioxopiperidin-3-yl)-1, 3-dioxoisoindolin-4-yl)amino)ethoxy)piperidin-1-yl)propanoyl)piperidin-4-yl)-2-(4-phenoxyphenyl)-4, 5, 6, 7-tetrahydropyrazolo[1, 5-a]pyrimidine-3-carboxamide

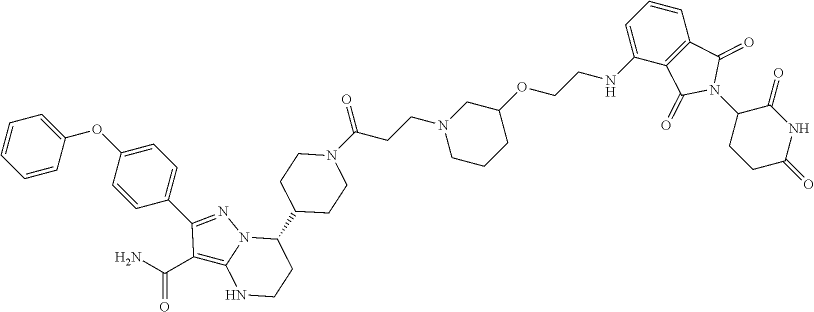 [SMILES: NC(=O)c1c(-c2ccc(Oc3ccccc3)cc2)nn2c1NCC[C@H]2C1CCN(C(=O)CCN2CCCC(OCCNc3cccc4c3C(=O)N(C3CCC(=O)NC3=O)C4=O)C2)CC1]

### Example 91: (7S)-7-(1-(1-(2-(2-((2-(2, 6-dioxopiperidin-3-yl)-1, 3-dioxoisoindolin-4-yl)amino)ethoxy)ethyl)piperidine-4-carbonyl)piperidin-4-yl)-2-(4-phenoxyphenyl)-4, 5, 6, 7-tetrahydropyrazolo[1,5-a]pyrimidine-3-carboxamide

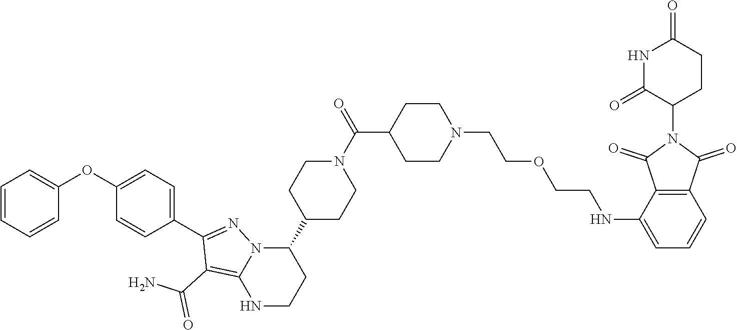 [SMILES: NC(=O)c1c(-c2ccc(Oc3ccccc3)cc2)nn2c1NCC[C@H]2C1CCN(C(=O)C2CCN(CCOCCNc3cccc4c3C(=O)N(C3CCC(=O)NC3=O)C4=O)CC2)CC1]

### Example 92: (7S)-7-(1-(3-(4-(2-((2-(2, 6-dioxopiperidin-3-yl)-1, 3-dioxoisoindolin-4-yl)amino)ethoxy)piperidin-1-yl)propanoyl)piperidin-4-yl)-2-(4-phenoxyphenyl)-4, 5, 6, 7-tetrahydropyrazolo[1, 5-a]pyrimidine-3-carboxamide

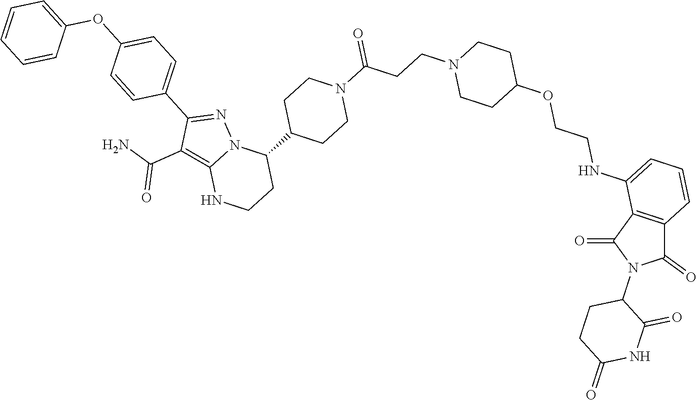 [SMILES: NC(=O)c1c(-c2ccc(Oc3ccccc3)cc2)nn2c1NCC[C@H]2C1CCN(C(=O)CCN2CCC(OCCNc3cccc4c3C(=O)N(C3CCC(=O)NC3=O)C4=O)CC2)CC1]

### Example 93: (7S)-7-(1-((1-(2-(2-((2-(2, 6-dioxopiperidin-3-yl)-1, 3-dioxoisoindolin-4-yl)amino)ethoxy)ethyl)piperidin-4-yl)carbamoyl)piperidin-4-yl)-2-(4-phenoxyphenyl)-4, 5, 6, 7-tetrahydropyrazolo[1,5-a]pyrimidine-3-carboxamide

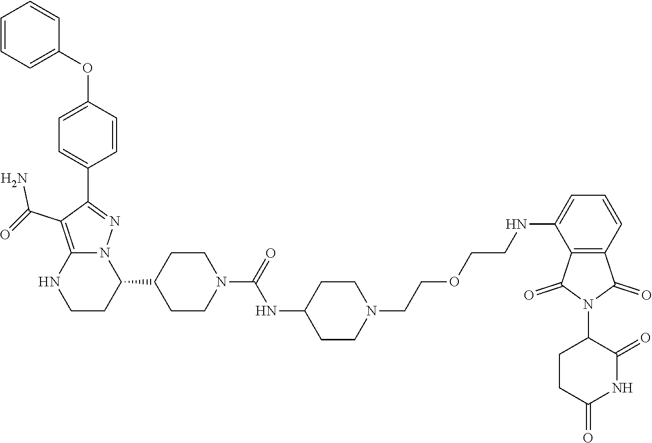 [SMILES: NC(=O)c1c(-c2ccc(Oc3ccccc3)cc2)nn2c1NCC[C@H]2C1CCN(C(=O)NC2CCN(CCOCCNc3cccc4c3C(=O)N(C3CCC(=O)NC3=O)C4=O)CC2)CC1]

### Example 94: (7S)-7-(1-((1-(2-(2-((2-(2, 6-dioxopiperidin-3-yl)-1, 3-dioxoisoindolin-4-yl)amino)ethoxy)ethyl)azetidin-3-yl)carbamoyl)piperidin-4-yl)-2-(4-phenoxyphenyl)-4, 5, 6, 7-tetrahydropyrazolo[1,5-a] pyrimidine-3-carboxamide

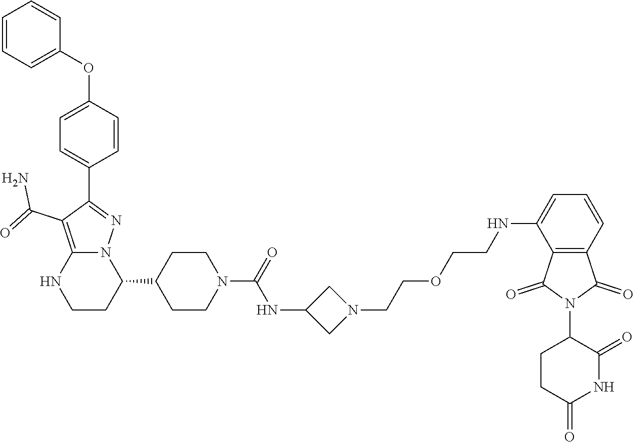 [SMILES: NC(=O)c1c(-c2ccc(Oc3ccccc3)cc2)nn2c1NCC[C@H]2C1CCN(C(=O)NC2CN(CCOCCNc3cccc4c3C(=O)N(C3CCC(=O)NC3=O)C4=O)C2)CC1]

### Example 95: (7S)-7-(1-(3-(4-((4-(2-(2, 6-dioxopiperidin-3-yl)-1, 3-dioxoisoindolin-5-yl)piperazin-1-yl)methyl)piperidin-1-yl)propanoyl)piperidin-4-yl)-2-(4-phenoxyphenyl)-4, 5, 6, 7-tetrahydropyrazolo[1,5-a]pyrimidine-3-carboxamide

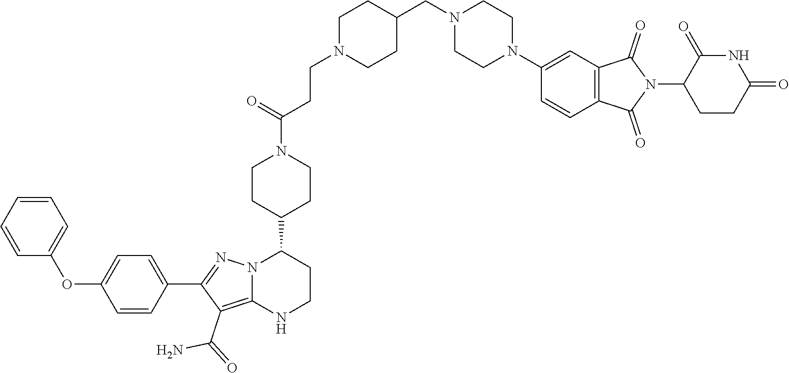 [SMILES: NC(=O)c1c(-c2ccc(Oc3ccccc3)cc2)nn2c1NCC[C@H]2C1CCN(C(=O)CCN2CCC(CN3CCN(c4ccc5c(c4)C(=O)N(C4CCC(=O)NC4=O)C5=O)CC3)CC2)CC1]

### Example 96: (S)-7-(1-(3-(4-((4-(4-(2, 4-dioxotetrahydropyrimidin-1(2H)-yl)phenyl)piperazin-1-yl)methyl)piperidin-1-yl)propanoyl)piperidin-4-yl)-2-(4-phenoxyphenyl)-4, 5, 6, 7-tetrahydropyrazolo[1,5-a]pyrimidine-3-carboxamide

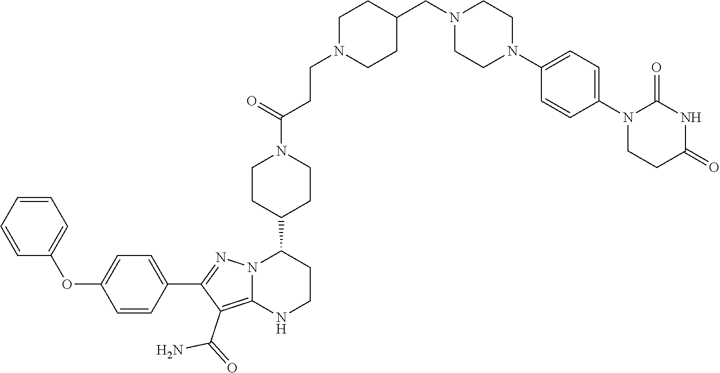 [SMILES: NC(=O)c1c(-c2ccc(Oc3ccccc3)cc2)nn2c1NCC[C@H]2C1CCN(C(=O)CCN2CCC(CN3CCN(c4ccc(N5CCC(=O)NC5=O)cc4)CC3)CC2)CC1]

### Example 97: (7S)-7-(1-(3-((2-(3-((2-(2, 6-dioxopiperidin-3-yl)-1, 3-dioxoisoindolin-5-yl)methyl)ureido)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

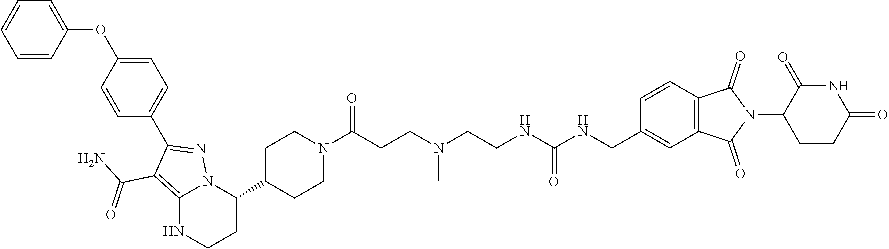 [SMILES: CN(CCNC(=O)NCc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1]

### Example 98: (7S)-7-(1-(3-((4-(3-((2-(2, 6-dioxopiperidin-3-yl)-1, 3-dioxoisoindolin-5-yl)methyl)ureido)cyclohexyl)(methyl)amino)propanoyl)piperidin-4-yl)-2-(4-phenoxyphenyl)-4, 5, 6, 7-tetrahydropyrazolo[1,5-a]pyrimidine-3-carboxamide

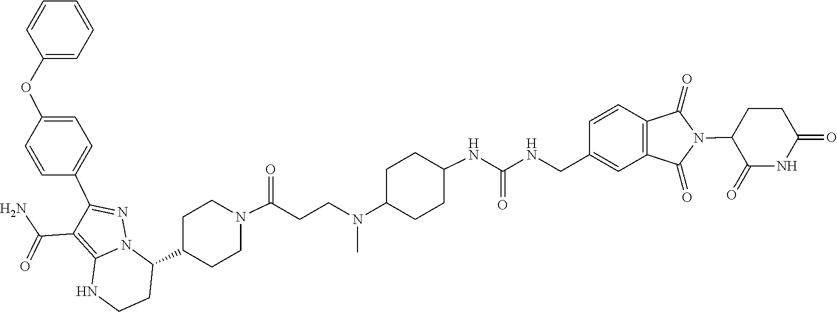 [SMILES: CN(CCC(=O)N1CCC([C@@H]2CCNc3c(C(N)=O)c(-c4ccc(Oc5ccccc5)cc4)nn32)CC1)C1CCC(NC(=O)NCc2ccc3c(c2)C(=O)N(C2CCC(=O)NC2=O)C3=O)CC1]

### Example 99: (7S)-7-(1-(3-(7-(2-(2, 6-dioxopiperidin-3-yl)-1, 3-dioxoisoindolin-5-yl)-2, 7-diazaspiro[3.5]nonan-2-yl)propanoyl)piperidin-4-yl)-2-(4-phenoxyphenyl)-4, 5, 6, 7-tetrahydropyrazolo[1,5-a]pyrimidine-3-carboxamide

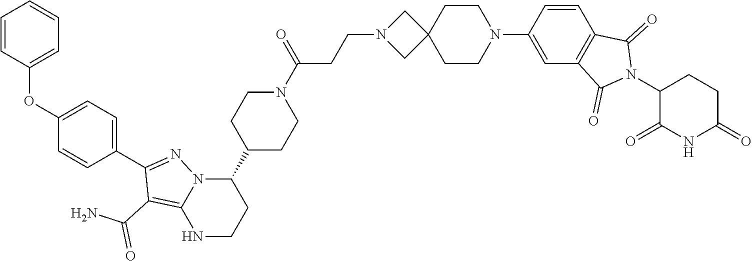

### Example 100: (7S)-7-(1-(3-(7-((2-(2, 6-dioxopiperidin-3-yl)-1, 3-dioxoisoindolin-5-yl)amino)-2-azaspiro[3.5]nonan-2-yl)propanoyl)piperidin-4-yl)-2-(4-phenoxyphenyl)-4, 5, 6, 7-tetrahydropyrazolo[1,5-a]pyrimidine-3-carboxamide

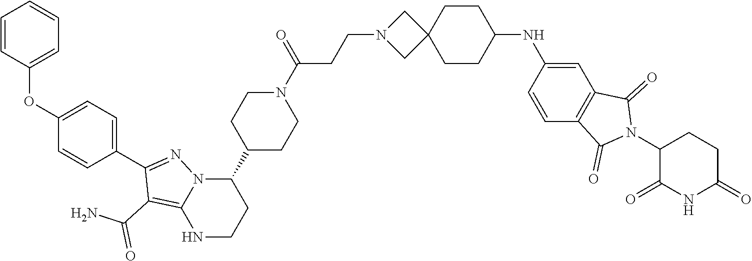

### Example 101: (S)-7-(1-(3-(7-((4-(2, 4-dioxotetrahydropyrimidin-1(2H)-yl)phenyl)amino)-2-azaspiro[3.5] nonan-2-yl)propanoyl)piperidin-4-yl)-2-(4-phenoxyphenyl)-4, 5, 6, 7-tetrahydropyrazolo[1,5-a]pyrimidine-3-carboxamide

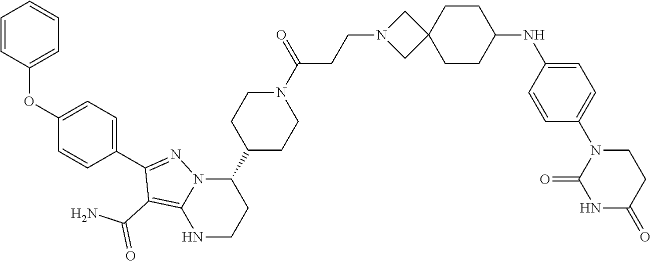

### Example 102: (S)-7-(1-(3-(7-(4-(2, 4-dioxotetrahydropyrimidin-1(2H)-yl)phenyl)-2, 7-diazaspiro[3.5]nonan-2-yl)propanoyl)piperidin-4-yl)-2-(4-phenoxyphenyl)-4, 5, 6, 7-tetrahydropyrazolo[1,5-a]pyrimidine-3-carboxamide

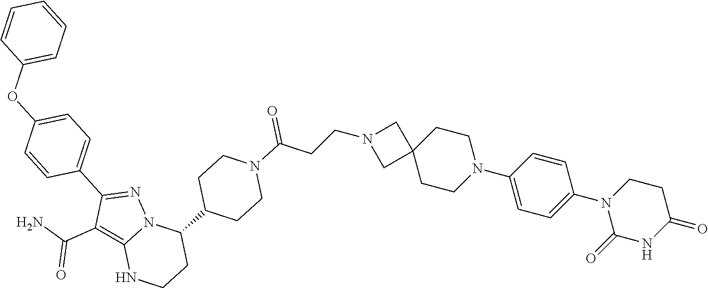 [SMILES: NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1]

### Example 103: (7S)-7-(1-(3-(((4-((2-(2, 6-dioxopiperidin-3-yl)-1, 3-dioxoisoindolin-5-yl)amino)cyclohexyl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

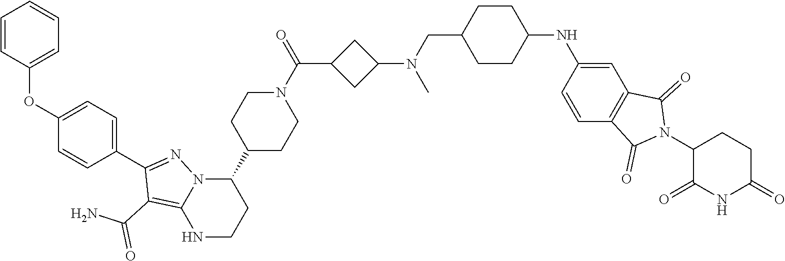

### Example 104: (7S)-7-(1-(3-(((1-(2-(2, 6-dioxopiperidin-3-yl)-1, 3-dioxoisoindolin-5-yl)piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

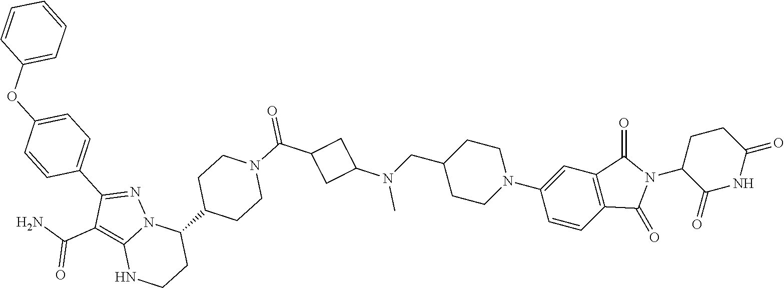 [SMILES: CN(CC1CCN(c2ccc3c(c2)C(=O)N(C2CCC(=O)NC2=O)C3=O)CC1)C1CC(C(=O)N2CCC([C@@H]3CCNc4c(C(N)=O)c(-c5ccc(Oc6ccccc6)cc5)nn43)CC2)C1]

### Example 105: (S)-7-(1-(3-(((1-(4-(2, 4-dioxotetrahydropyrimidin-1(2H)-yl)phenyl)piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

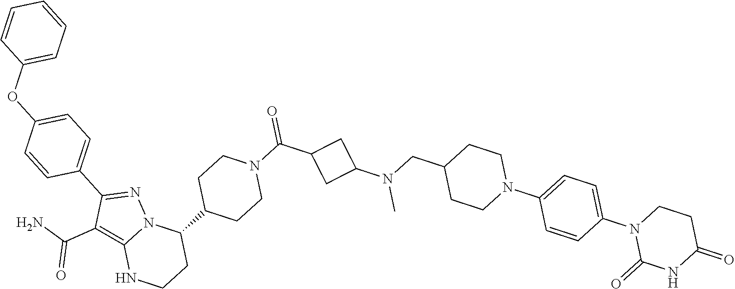

### Example 106: (S)-7-(1-(3-(((1-(3-(2, 4-dioxotetrahydropyrimidin-1(2H)-yl)phenyl)piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

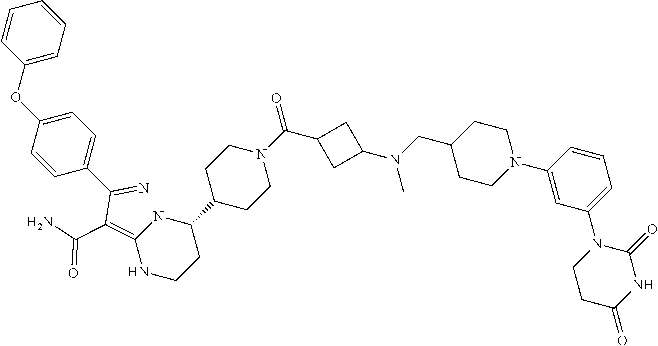 [SMILES: CN(CC1CCN(c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1]

### Example 107: (S)-7-(1-(3-(((1-(3-(2, 4-dioxotetrahydropyrimidin-1(2H)-yl)benzoyl)piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

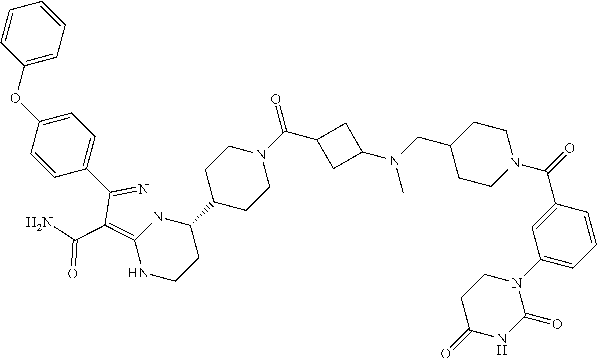 [SMILES: CN(CC1CCN(C(=O)c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1]

### Example 108: (S)-7-(1-(3-(methyl((1-(7-oxo-5, 6, 7, 8-tetrahydro-1, 8-naphthyridine-4-carbonyl)piperidin-4-yl)methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

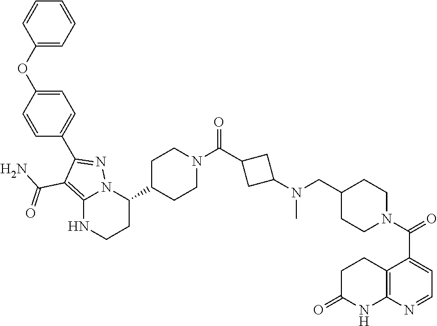 [SMILES: CN(CC1CCN(C(=O)c2ccnc3c2CCC(=O)N3)CC1)C1CC(C(=O)N2CCC([C@@H]3CCNc4c(C(N)=O)c(-c5ccc(Oc6ccccc6)cc5)nn43)CC2)C1]

### Example 109: (S)-7-(1-(3-(methyl((1-(7-oxo-5, 6, 7, 8-tetrahydro-1, 8-naphthyridine-3-carbonyl)piperidin-4-yl)methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

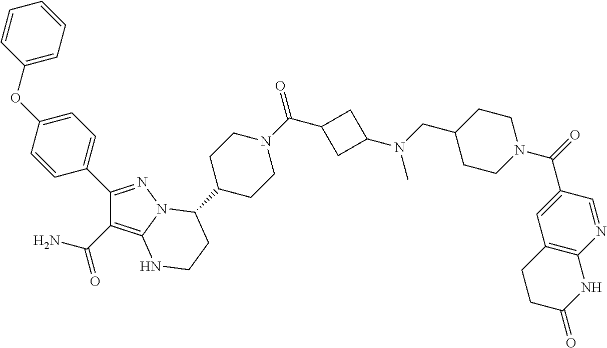 [SMILES: CN(CC1CCN(C(=O)c2cnc3c(c2)CCC(=O)N3)CC1)C1CC(C(=O)N2CCC([C@@H]3CCNc4c(C(N)=O)c(-c5ccc(Oc6ccccc6)cc5)nn43)CC2)C1]

### Example 110: (7S)-7-(4-(4-((1-(2-(2, 6-dioxopiperidin-3-yl)-1, 3-dioxoisoindolin-5-yl)piperidin-4-yl)methyl)piperazin-1-yl)cyclohexyl)-2-(4-phenoxyphenyl)-4, 5, 6, 7-tetrahydropyrazolo[1,5-a]pyrimidine-3-carboxamide

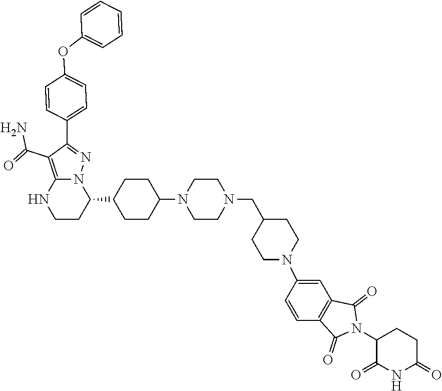 [SMILES: NC(=O)c1c(-c2ccc(Oc3ccccc3)cc2)nn2c1NCC[C@H]2C1CCC(N2CCN(CC3CCN(c4ccc5c(c4)C(=O)N(C4CCC(=O)NC4=O)C5=O)CC3)CC2)CC1]

### Example 111: (7S)-7-(4-((1-((1-(2-(2, 6-dioxopiperidin-3-yl)-1, 3-dioxoisoindolin-5-yl)piperidin-4-yl)methyl)piperidin-4-yl)amino)cyclohexyl)-2-(4-phenoxyphenyl)-4, 5, 6, 7-tetrahydropyrazolo[1,5-a] pyrimidine-3-carboxamide

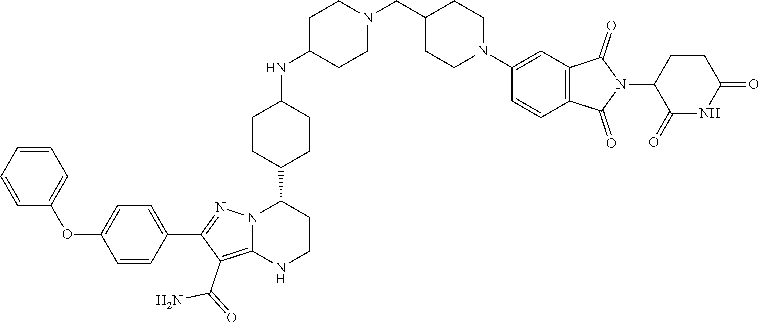 [SMILES: NC(=O)c1c(-c2ccc(Oc3ccccc3)cc2)nn2c1NCC[C@H]2C1CCC(NC2CCN(CC3CCN(c4ccc5c(c4)C(=O)N(C4CCC(=O)NC4=O)C5=O)CC3)CC2)CC1]

### Example 112: (7S)-7-(4-(4-((1-(4-(2, 4-dioxotetrahydropyrimidin-1(2H)-yl)phenyl)piperidin-4-yl)methyl)piperazin-1-yl)cyclohexyl)-2-(4-phenoxyphenyl)-4, 5, 6, 7-tetrahydropyrazolo[1,5-a]pyrimidine-3-carboxamide

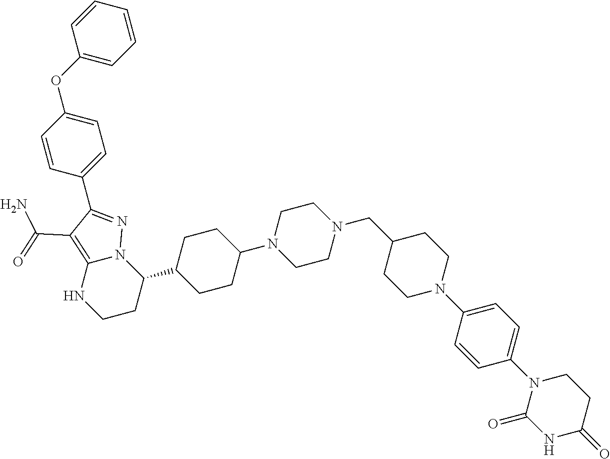 [SMILES: NC(=O)c1c(-c2ccc(Oc3ccccc3)cc2)nn2c1NCC[C@H]2C1CCC(N2CCN(CC3CCN(c4ccc(N5CCC(=O)NC5=O)cc4)CC3)CC2)CC1]

### Example 113: (7S)-7-(4-(4-((1-(2-(2, 6-dioxopiperidin-3-yl)-1, 3-dioxoisoindoline-5-carbonyl)piperidin-4-yl)methyl)piperazin-1-yl)cyclohexyl)-2-(4-phenoxyphenyl)-4, 5, 6, 7-tetrahydropyrazolo[1,5-a] pyrimidine-3-carboxamide

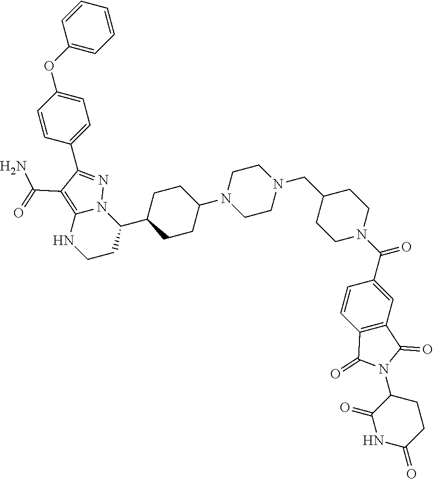

### Example 114: (7S)-7-(4-(4-(4-(1-(2-(2, 6-dioxopiperidin-3-yl)-1, 3-dioxoisoindoline-5-carbonyl)piperidin-4-yl)benzyl)piperazin-1-yl)cyclohexyl)-2-(4-phenoxyphenyl)-4, 5, 6, 7-tetrahydropyrazolo[1,5-a] pyrimidine-3-carboxamide

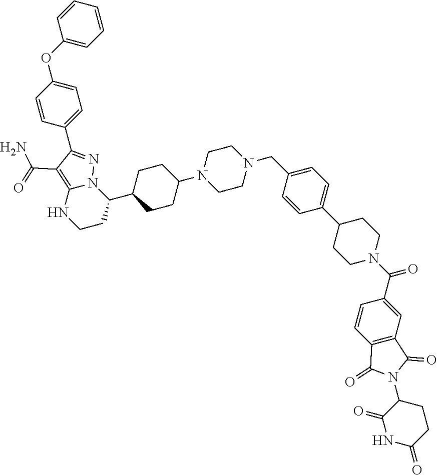

### Example 115: (7S)-7-(4-(4-(4-(4-(2-(2, 6-dioxopiperidin-3-yl)-1, 3-dioxoisoindoline-5-carbonyl)piperazin-1-yl)benzyl)piperazin-1-yl)cyclohexyl)-2-(4-phenoxyphenyl)-4, 5, 6, 7-tetrahydropyrazolo[1,5-a]pyrimidine-3-carboxamide

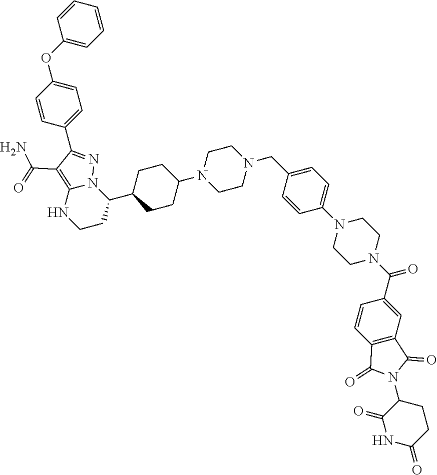

### Example 116: (7S)-7-(4-(4-((1-(3-(2, 4-dioxotetrahydropyrimidin-1(2H)-yl)benzoyl)piperidin-4-yl)methyl)piperazin-1-yl)cyclohexyl)-2-(4-phenoxyphenyl)-4, 5, 6, 7-tetrahydropyrazolo[1,5-a]pyrimidine-3-carboxamide

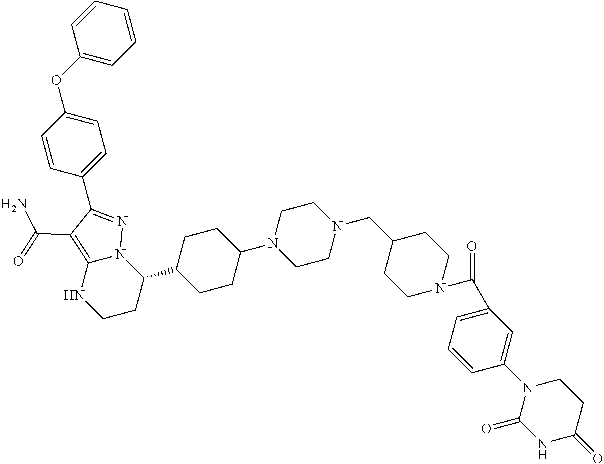 [SMILES: NC(=O)c1c(-c2ccc(Oc3ccccc3)cc2)nn2c1NCC[C@H]2C1CCC(N2CCN(CC3CCN(C(=O)c4cccc(N5CCC(=O)NC5=O)c4)CC3)CC2)CC1]

### Example 117: (7S)-7-(4-(4-(4-(1-(3-(2, 4-dioxotetrahydropyrimidin-1(2H)-yl)benzoyl)piperidin-4-yl)benzyl)piperazin-1-yl)cyclohexyl)-2-(4-phenoxyphenyl)-4, 5, 6, 7-tetrahydropyrazolo[1,5-a]pyrimidine-3-carboxamide

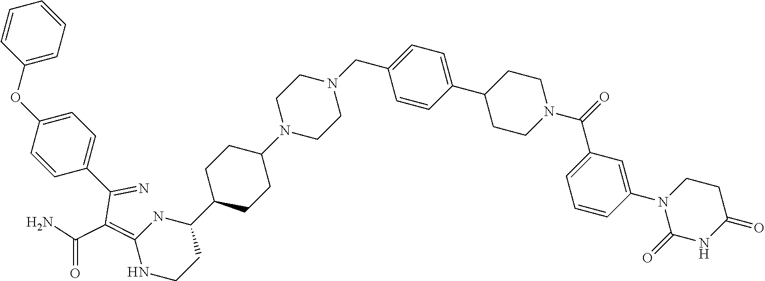

### Example 118: (S)-7-(1-(3-((2-(4-(3-(2, 4-dioxotetrahydropyrimidin-1(2H)-yl)phenyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

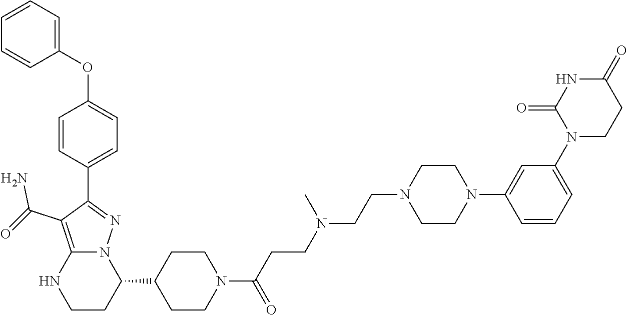 [SMILES: CN(CCC(=O)N1CCC([C@@H]2CCNc3c(C(N)=O)c(-c4ccc(Oc5ccccc5)cc4)nn32)CC1)CCN1CCN(c2cccc(N3CCC(=O)NC3=O)c2)CC1]

### Example 119: (7S)-7-(4-(4-(4-(4-(3-(2, 4-dioxotetrahydropyrimidin-1(2H)-yl)benzoyl)piperazin-1-yl)benzyl)piperazin-1-yl)cyclohexyl)-2-(4-phenoxyphenyl)-4, 5, 6, 7-tetrahydropyrazolo[1,5-a]pyrimidine-3-carboxamide

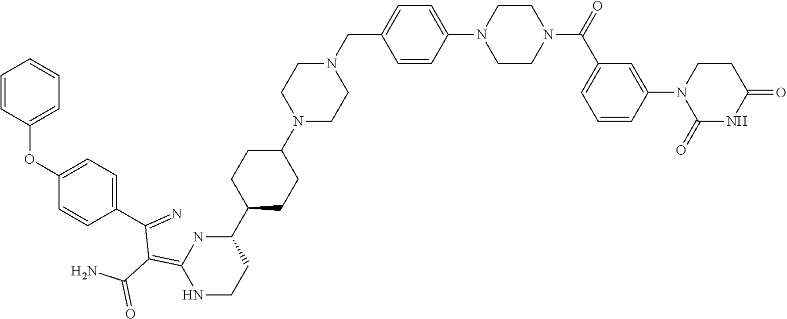

### Example 120: (7S)-7-(4-((1-((1-(3-(2, 4-dioxotetrahydropyrimidin-1(2H)-yl)phenyl)piperidin-4-yl)methyl)piperidin-4-yl)amino)cyclohexyl)-2-(4-phenoxyphenyl)-4, 5, 6, 7-tetrahydropyrazolo[1,5-a]pyrimidine-3-carboxamide

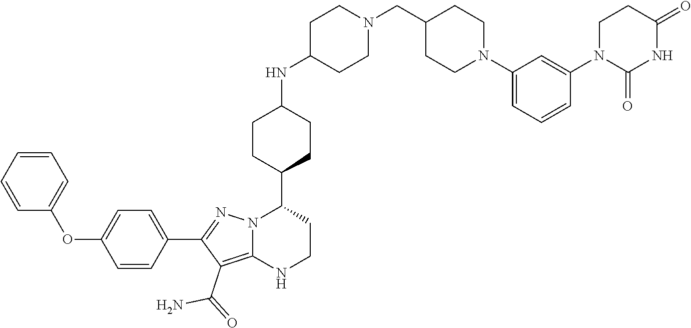

### Example 121: (7S)-7-(4-((1-((1-(2-(2, 6-dioxopiperidin-3-yl)-1, 3-dioxoisoindoline-5-carbonyl)piperidin-4-yl)methyl)piperidin-4-yl)amino)cyclohexyl)-2-(4-phenoxyphenyl)-4, 5, 6, 7-tetrahydropyrazolo[1,5-a]pyrimidine-3-carboxamide

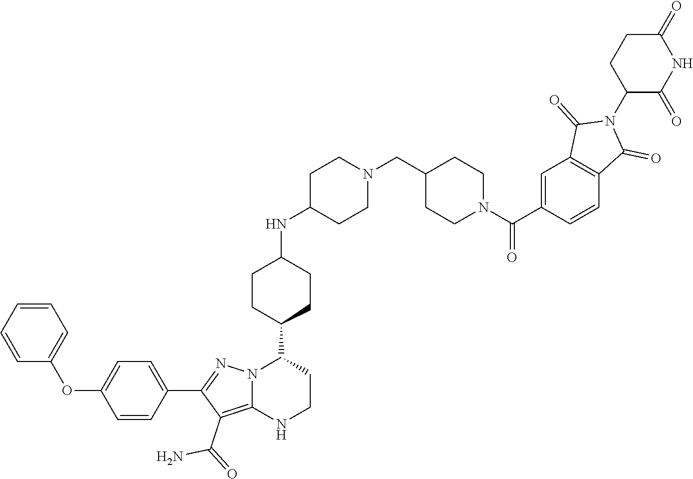

### Example 122: (S)-7-(1-(3-((2-(4-(3-(2, 4-dioxotetrahydropyrimidin-1(2H)-yl)benzoyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

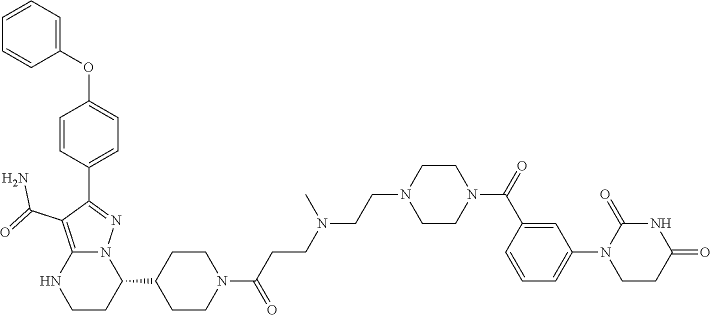 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cccc(N3CCC(=O)NC3=O)c2)CC1]

### Example 123: (7S)-7-(4-((1-(4-(1-(3-(2, 4-dioxotetrahydropyrimidin-1(2H)-yl)phenyl)piperidin-4-yl)benzyl)piperidin-4-yl)amino)cyclohexyl)-2-(4-phenoxyphenyl)-4, 5, 6, 7-tetrahydropyrazolo[1,5-a]pyrimidine-3-carboxamide

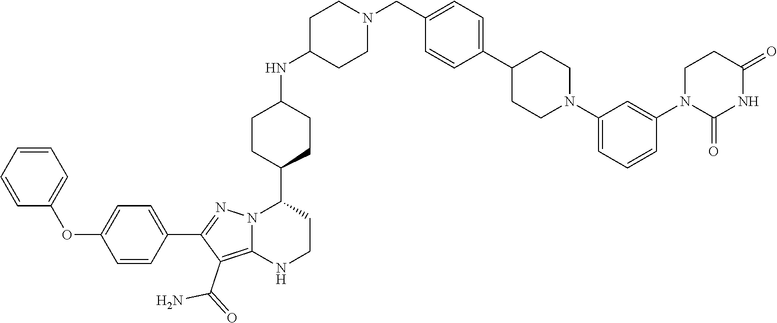

### Example 124: (7S)-7-(4-((1-(4-(4-(3-(2, 4-dioxotetrahydropyrimidin-1(2H)-yl)phenyl)piperazin-1-yl)benzyl)piperidin-4-yl)amino)cyclohexyl)-2-(4-phenoxyphenyl)-4, 5, 6, 7-tetrahydropyrazolo[1,5-a]pyrimidine-3-carboxamide

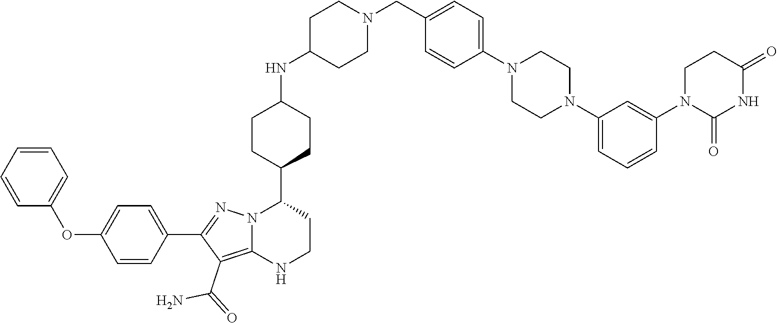

### Example 125: (7S)-7-(4-((1-(4-(1-(2-(2, 6-dioxopiperidin-3-yl)-1, 3-dioxoisoindoline-5-carbonyl)piperidin-4-yl)benzyl)piperidin-4-yl)amino)cyclohexyl)-2-(4-phenoxyphenyl)-4, 5, 6, 7-tetrahydropyrazolo[1,5-a] pyrimidine-3-carboxamide

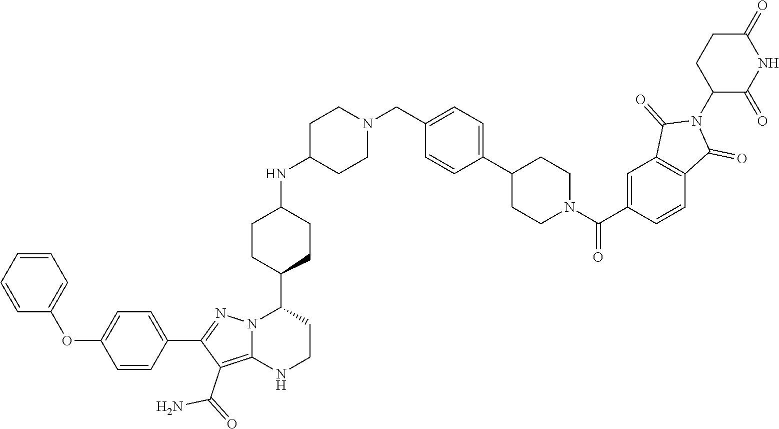

### Example 126: (S)-7-(1-(3-(7-(3-(2, 4-dioxotetrahydropyrimidin-1(2H)-yl)benzamido)-2-azaspiro[3,5]nonan-2-yl)propanoyl)piperidin-4-yl)-2-(4-phenoxyphenyl)-4, 5, 6, 7-tetrahydropyrazolo[1,5-a]pyrimidine-3-carboxamide

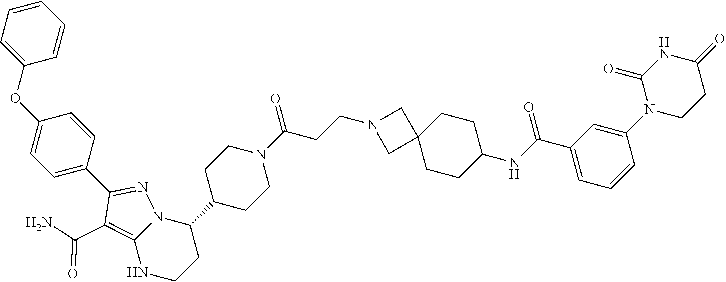 [SMILES: NC(=O)c1c(-c2ccc(Oc3ccccc3)cc2)nn2c1NCC[C@H]2C1CCN(C(=O)CCN2CC3(CCC(NC(=O)c4cccc(N5CCC(=O)NC5=O)c4)CC3)C2)CC1]

### Example 127: (7S)-7-(4-((1-(4-(4-(2-(2, 6-dioxopiperidin-3-yl)-1, 3-dioxoisoindoline-5-carbonyl)piperazin-1-yl)benzyl)piperidin-4-yl)amino)cyclohexyl)-2-(4-phenoxyphenyl)-4, 5, 6, 7-tetrahydropyrazolo[1,5-a]pyrimidine-3-carboxamide

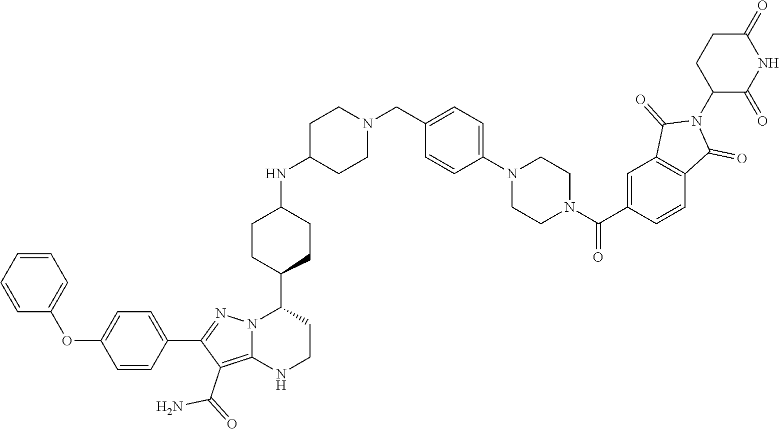

### Example 128: (S)-7-(1-(3-(7-(3-(2, 4-dioxotetrahydropyrimidin-1(2H)-yl)benzoyl)-2, 7-diazaspiro[3.5]nonan-2-yl)propanoyl)piperidin-4-yl)-2-(4-phenoxyphenyl)-4, 5, 6, 7-tetrahydropyrazolo[1,5-a] pyrimidine-3-carboxamide

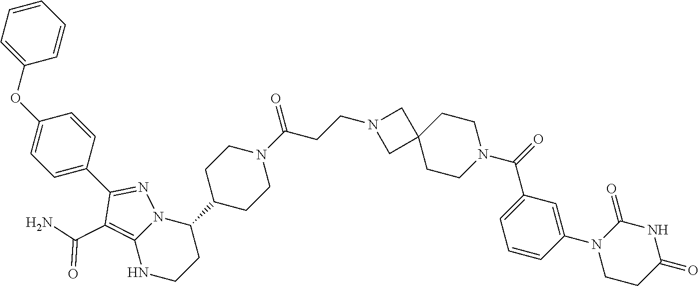 [SMILES: NC(=O)c1c(-c2ccc(Oc3ccccc3)cc2)nn2c1NCC[C@H]2C1CCN(C(=O)CCN2CC3(CCN(C(=O)c4cccc(N5CCC(=O)NC5=O)c4)CC3)C2)CC1]

### Example 129: (S)-7-(1-(3-((4-(4-(3-(2, 4-dioxotetrahydropyrimidin-1(2H)-yl)phenyl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

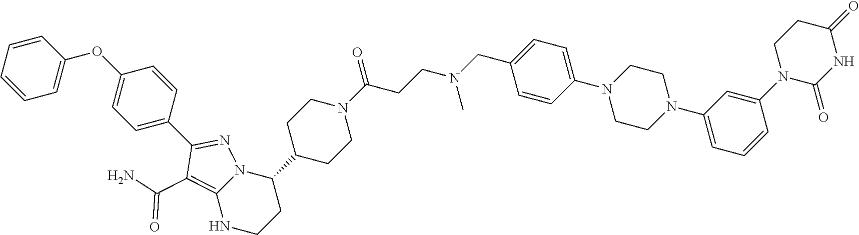 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3cccc(N4CCC(=O)NC4=O)c3)CC2)cc1]

### Example 130: (S)-7-(1-(3-(((6-(4-(4-(2, 4-dioxotetrahydropyrimidin-1(2H)-yl)benzoyl)piperazin-1-yl)pyridin-3-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

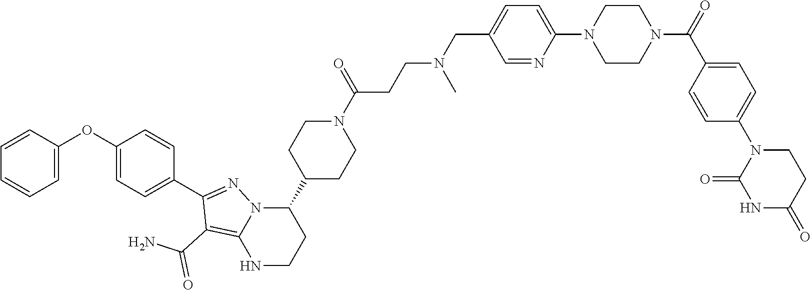 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)nc1]

### Example 131: (7S)-7-(1-(3-((2-(4-(3-((2, 6-dioxopiperidin-3-yl)amino)phenyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

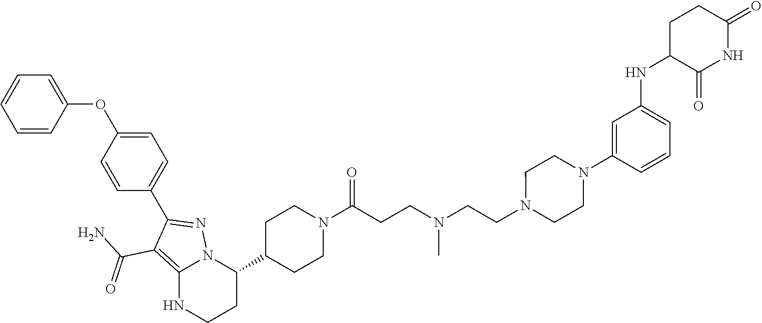 [SMILES: CN(CCC(=O)N1CCC([C@@H]2CCNc3c(C(N)=O)c(-c4ccc(Oc5ccccc5)cc4)nn32)CC1)CCN1CCN(c2cccc(NC3CCC(=O)NC3=O)c2)CC1]

### Example 132: (7S)-7-(1-(3-((2-(4-(3-((2, 6-dioxopiperidin-3-yl)amino)benzoyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

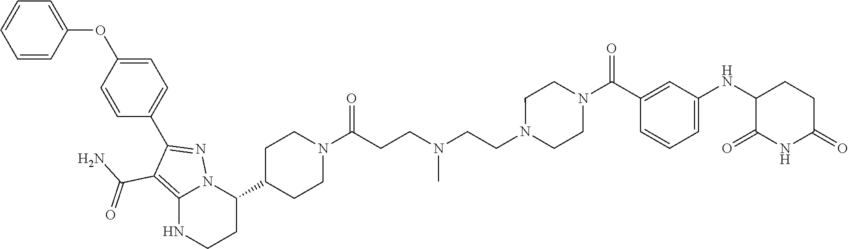 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cccc(NC3CCC(=O)NC3=O)c2)CC1]

### Example 133: (S)-7-(1-(3-((4-(4-(3-(2, 4-dioxotetrahydropyrimidin-1(2H)-yl)benzoyl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

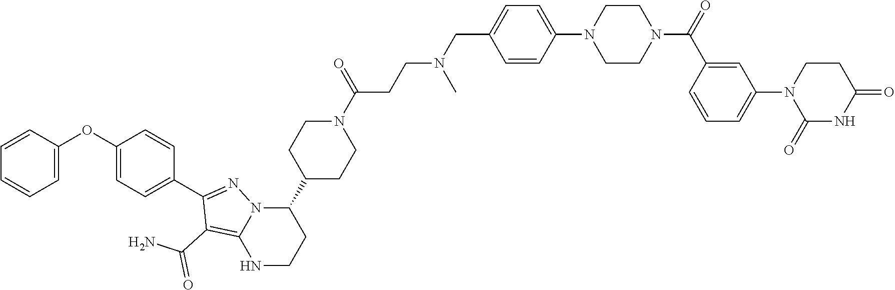 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cc1]

### Example 134: (S)-7-(1-(3-((4-(4-(5-(2, 4-dioxotetrahydropyrimidin-1(2H)-yl)picolinoyl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

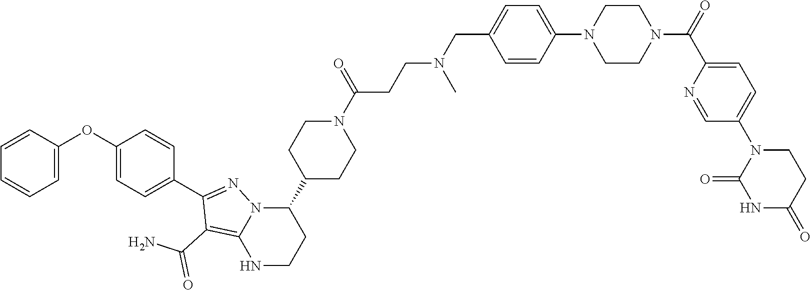 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cn3)CC2)cc1]

### Example 135: (7S)-7-(1-(3-((2-(4-(4-((2, 6-dioxopiperidin-3-yl)amino)picolinoyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

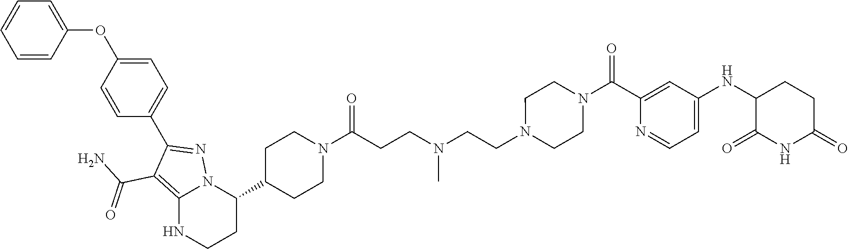 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccn2)CC1]

### Example 136: (S)-7-(1-(3-(((5-(4-(3-(2, 4-dioxotetrahydropyrimidin-1(2H)-yl)benzoyl)piperazin-1-yl)pyridin-2-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

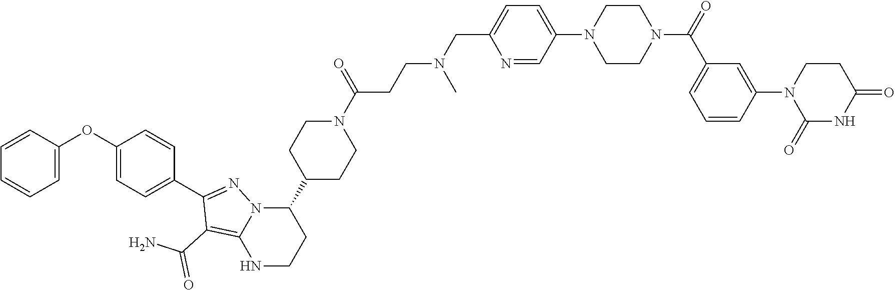 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cn1]

### Example 137: (S)-7-(1-(3-(((5-(4-(4-(2, 4-dioxotetrahydropyrimidin-1(2H)-yl)benzoyl)piperazin-1-yl)pyridin-2-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

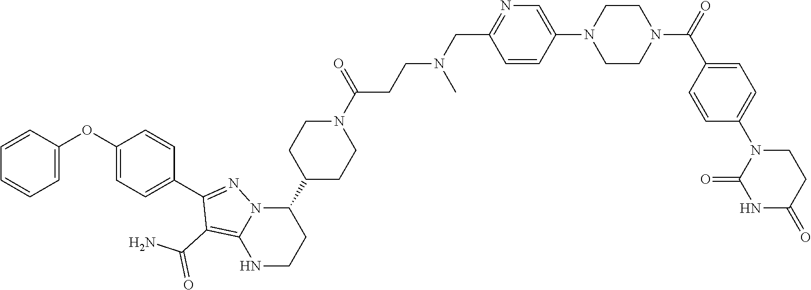 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)cn1]

### Example 138: (7S)-7-(1-(3-((2-(4-(5-((2, 6-dioxopiperidin-3-yl)amino)-2-fluorobenzoyl)piperazin-1-yl)ethyl)(methyl)amino)propanoyl)piperidin-4-yl)-2-(4-phenoxyphenyl)-4, 5, 6, 7-tetrahydropyrazolo[1,5-a]pyrimidine-3-carboxamide

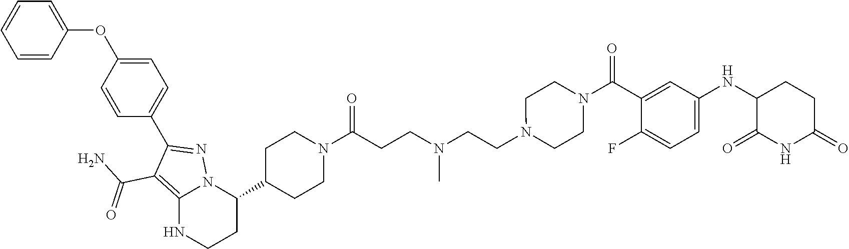 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccc2F)CC1]

### Example 139: (S)-7-(1-(3-((4-(4-(5-(2, 4-dioxotetrahydropyrimidin-1(2H)-yl)-2-fluorobenzoyl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

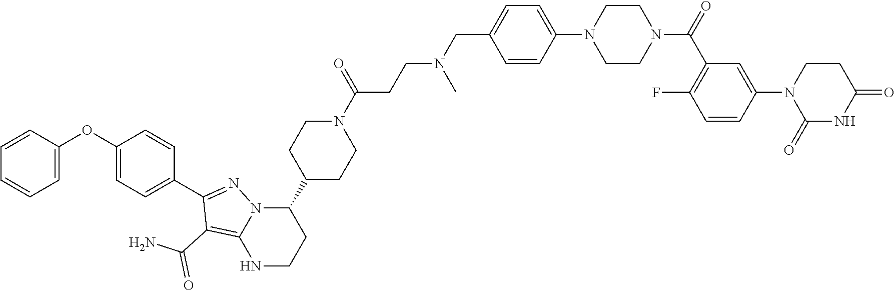 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cc(N4CCC(=O)NC4=O)ccc3F)CC2)cc1]

### Example 140: (S)-7-(1-(3-(((5-(4-(4-(2, 4-dioxotetrahydropyrimidin-1(2H)-yl)benzoyl)piperazin-1-yl)pyrimidin-2-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

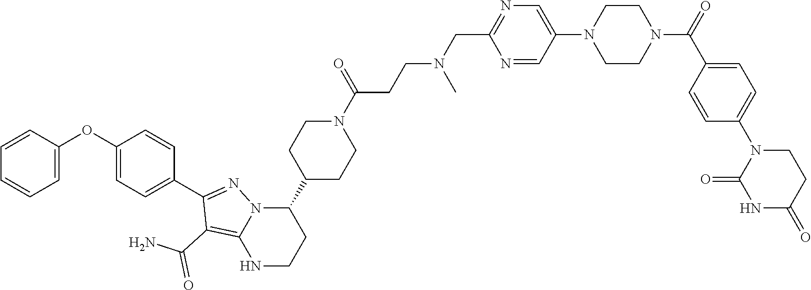 [SMILES: CN(CCC(=O)N1CCC([C@@H]2CCNc3c(C(N)=O)c(-c4ccc(Oc5ccccc5)cc4)nn32)CC1)Cc1ncc(N2CCN(C(=O)c3ccc(N4CCC(=O)NC4=O)cc3)CC2)cn1]

### Example 141: (S)-7-(1-(3-(((1-(4-(2, 4-dioxotetrahydropyrimidin-1(2H)-yl)benzoyl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

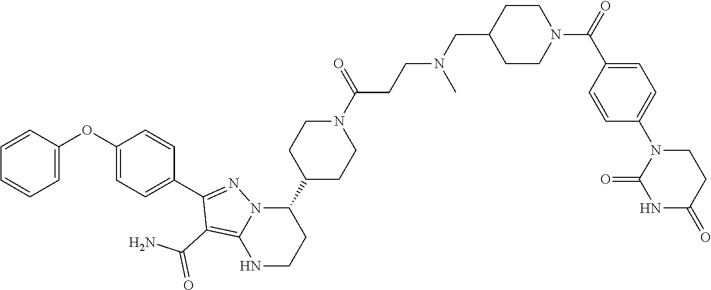 [SMILES: CN(CCC(=O)N1CCC([C@@H]2CCNc3c(C(N)=O)c(-c4ccc(Oc5ccccc5)cc4)nn32)CC1)CC1CCN(C(=O)c2ccc(N3CCC(=O)NC3=O)cc2)CC1]

### Example 142: (S)-7-(1-(3-(((1-(3-(2, 4-dioxotetrahydropyrimidin-1(2H)-yl)benzoyl)piperidin-4-yl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

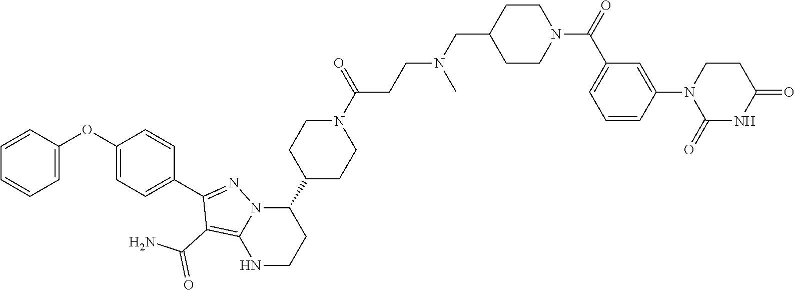 [SMILES: CN(CCC(=O)N1CCC([C@@H]2CCNc3c(C(N)=O)c(-c4ccc(Oc5ccccc5)cc4)nn32)CC1)CC1CCN(C(=O)c2cccc(N3CCC(=O)NC3=O)c2)CC1]

### Example 143: (S)-7-(1-(3-(7-((3-(2, 4-dioxotetrahydropyrimidin-1(2H)-yl)phenyl)amino)-2-azaspiro[3,5]nonan-2-yl)propanoyl)piperidin-4-yl)-2-(4-phenoxyphenyl)-4, 5, 6, 7-tetrahydropyrazolo[1,5-a]pyrimidine-3-carboxamide

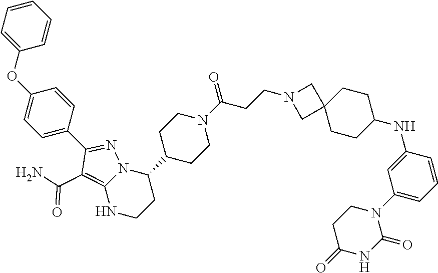 [SMILES: NC(=O)c1c(-c2ccc(Oc3ccccc3)cc2)nn2c1NCC[C@H]2C1CCN(C(=O)CCN2CC3(CCC(Nc4cccc(N5CCC(=O)NC5=O)c4)CC3)C2)CC1]

### Example 144: (S)-7-(1-(3-(7-(3-(2, 4-dioxotetrahydropyrimidin-1(2H)-yl)phenyl)-2, 7-diazaspiro[3,5]nonan-2-yl)propanoyl)piperidin-4-yl)-2-(4-phenoxyphenyl)-4, 5, 6, 7-tetrahydropyrazolo[1,5-a]pyrimidine-3-carboxamide

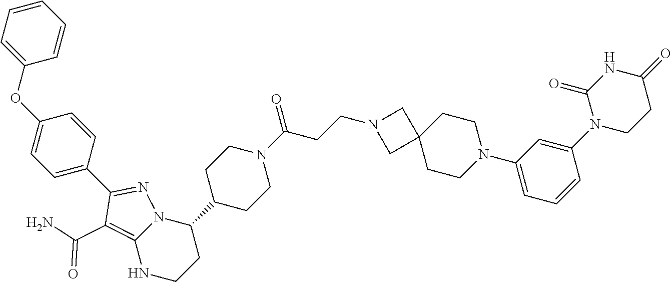 [SMILES: NC(=O)c1c(-c2ccc(Oc3ccccc3)cc2)nn2c1NCC[C@H]2C1CCN(C(=O)CCN2CC3(CCN(c4cccc(N5CCC(=O)NC5=O)c4)CC3)C2)CC1]

### Example 145: (S)-7-(1-(3-(((4-(4-(3-(2, 4-dioxotetrahydropyrimidin-1(2H)-yl)benzoyl)piperazin-1-yl)cyclohexyl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

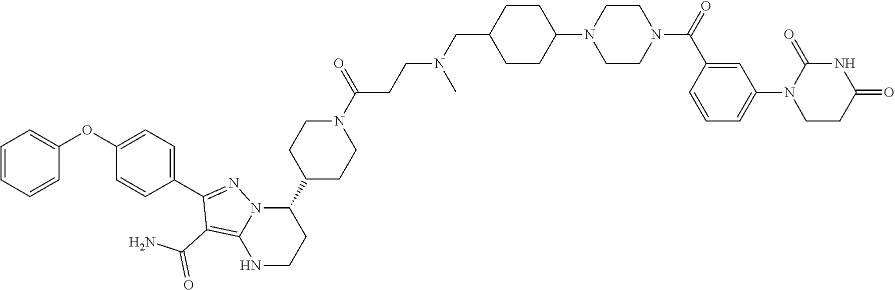 [SMILES: CN(CCC(=O)N1CCC([C@@H]2CCNc3c(C(N)=O)c(-c4ccc(Oc5ccccc5)cc4)nn32)CC1)CC1CCC(N2CCN(C(=O)c3cccc(N4CCC(=O)NC4=O)c3)CC2)CC1]

### Example 146: (S)-7-(1-(3-(((4-(4-(3-(2, 4-dioxotetrahydropyrimidin-1(2H)-yl)phenyl)piperazin-1-yl)cyclohexyl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

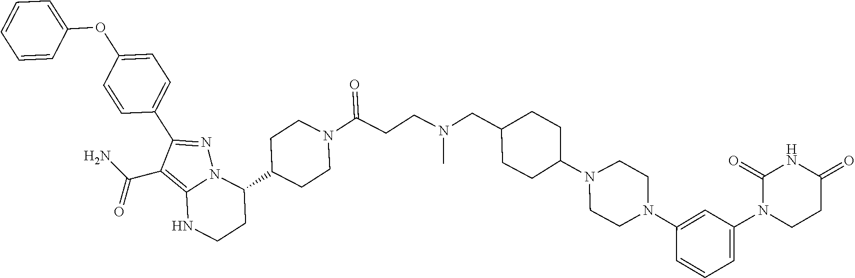 [SMILES: CN(CCC(=O)N1CCC([C@@H]2CCNc3c(C(N)=O)c(-c4ccc(Oc5ccccc5)cc4)nn32)CC1)CC1CCC(N2CCN(c3cccc(N4CCC(=O)NC4=O)c3)CC2)CC1]

### Example 147: (7R)-7-(1-(2-(4-(4-(2-(2, 6-dioxopiperidin-3-yl)-1, 3-dioxoisoindolin-5-yl)piperazin-1-yl)cyclohexyl)acetyl)piperidin-4-yl)-2-(4-phenoxyphenyl)-4, 5, 6, 7-tetrahydropyrazolo[1,5-a]pyrimidine-3-carboxamide

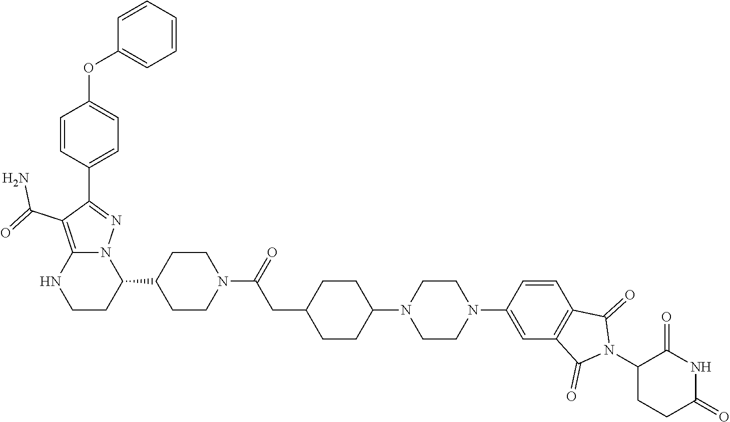 [SMILES: NC(=O)c1c(-c2ccc(Oc3ccccc3)cc2)nn2c1NCC[C@H]2C1CCN(C(=O)CC2CCC(N3CCN(c4ccc5c(c4)C(=O)N(C4CCC(=O)NC4=O)C5=O)CC3)CC2)CC1]

The titled compound was synthesized in the procedures similar to Example 151. ¹H NMR (400 MHz, DMSO) δ_{H} 11.08 (s, 1H), 7.67 (d, J=8.4 Hz, 1H), 7.50 (d, J=8.5 Hz, 2H), 7.41 (t, J=8.0 Hz, 2H), 7.32 (s, 1H), 7.24 (d, J=8.4 Hz, 1H), 7.17 (t, J=7.5 Hz, 1H), 7.10-7.02 (m, 4H), 6.68 (s, 1H), 5.10-5.01 (m, 1H), 4.46 (d, J=10.0 Hz, 1H), 4.01 (s, 2H), 3.40 (s, 4H), 3.30 (s, 2H), 3.00-2.80 (m, 2H), 2.59 (d, J=15.8 Hz, 5H), 2.50-2.37 (m, 2H), 2.30-2.15 (m, 4H), 2.07-1.85 (m, 4H), 1.84-1.69 (m, 2H), 1.69-1.60 (m, 2H), 1.55 (d, J=11.9 Hz, 1H), 1.51-1.37 (m, 4H), 1.30-1.10 (m, 3H); [M+H]⁺=881.8.

### Example 148: (7S)-7-(1-(4-(4-(4-(2-(2, 6-dioxopiperidin-3-yl)-1, 3-dioxoisoindolin-5-yl)piperazin-1-yl)cyclohexyl)butanoyl)piperidin-4-yl)-2-(4-phenoxyphenyl)-4, 5, 6, 7-tetrahydropyrazolo[1,5-a]pyrimidine-3-carboxamide

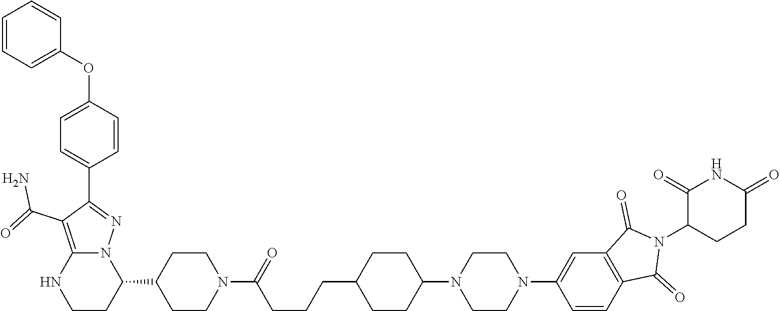 [SMILES: NC(=O)c1c(-c2ccc(Oc3ccccc3)cc2)nn2c1NCC[C@H]2C1CCN(C(=O)CCCC2CCC(N3CCN(c4ccc5c(c4)C(=O)N(C4CCC(=O)NC4=O)C5=O)CC3)CC2)CC1]

The titled compound was synthesized in the procedures similar to Example 151. ¹H NMR (400 MHz, DMSO) δ₁₁ 10.81 (s, 1H), 7.64 (d, J=8.5 Hz, 1H), 7.50 (d, J=8.1 Hz, 2H), 7.40 (t, J=7.7 Hz, 2H), 7.27 (s, 1H), 7.23-7.12 (m, 2H), 7.05 (t, J=7.2 Hz, 4H), 6.57 (s, 1H), 5.88 (s, 2H), 5.04-4.99 (m, 1H), 4.01 (d, J=5.4 Hz, 1H), 3.41 (s, 4H), 3.32 (s, 2H), 2.92-2.80 (m, 2H), 2.68-2.52 (m, 7H), 2.32-2.18 (m, 4H), 2.08-1.90 (m, 3H), 1.85-1.53 (m, 5H), 1.53-1.38 (m, 7H), 1.35-1.15 (m, 6H); [M+H]⁺=910.6.

### Example 149: (7S)-7-(1-(5-((4-(2-((2-(2, 6-dioxopiperidin-3-yl)-1, 3-dioxoisoindolin-4-yl)oxy)acetamido)butyl)amino)-5-oxopentanoyl)piperidin-4-yl)-2-(4-phenoxyphenyl)-4, 5, 6, 7-tetrahydropyrazolo[1,5-a]pyrimidine-3-carboxamide

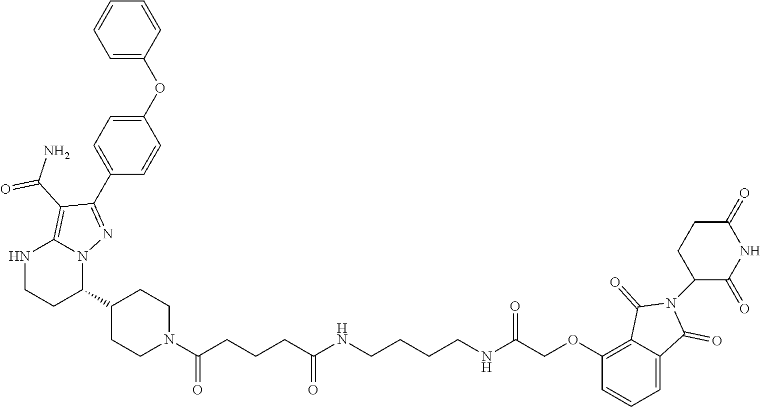 [SMILES: NC(=O)c1c(-c2ccc(Oc3ccccc3)cc2)nn2c1NCC[C@H]2C1CCN(C(=O)CCCC(=O)NCCCCNC(=O)COc2cccc3c2C(=O)N(C2CCC(=O)NC2=O)C3=O)CC1]

The titled compound was synthesized in the procedures similar to Example 23. ¹H NMR (400 MHz, DMSO) δ_{H} 11.09 (s, 1H), 7.93 (s, 1H), 7.84-7.77 (m, 1H), 7.72 (s, 1H), 7.49 (dd, J=7.8, 3.7 Hz, 3H), 7.40 (dd, J=15.1, 8.0 Hz, 3H), 7.17 (t, J=7.2 Hz, 1H), 7.06 (t, J=8.9 Hz, 4H), 6.65 (s, 1H), 5.11 (dd, J=12.9, 5.2 Hz, 1H), 4.76 (s, 2H), 4.45 (s, 1H), 4.00 (s, 1H), 3.87 (s, 1H), 3.18-3.06 (m, 4H), 3.02 (d, J=6.0 Hz, 2H), 2.90 (dd, J=19.0, 12.8 Hz, 2H), 2.68-2.53 (m, 2H), 2.43 (s, 1H), 2.26 (s, 3H), 2.06 (dd, J=17.6, 10.2 Hz, 4H), 1.92 (s, 1H), 1.75-1.63 (m, 3H), 1.52 (d, J=12.5 Hz, 1H), 1.41 (s, 4H), 1.24 (s, 1H); [M+H]⁺=915.8.

### Example 150: (7S)-7-(1′-(4-(4-(2-(2, 6-dioxopiperidin-3-yl)-1, 3-dioxoisoindolin-5-yl)piperazine-1-carbonyl)benzyl)-[1, 4′-bipiperidin]-4-yl)-2-(4-phenoxyphenyl)-4, 5, 6, 7-tetrahydropyrazolo[1,5-a] pyrimidine-3-carboxamide

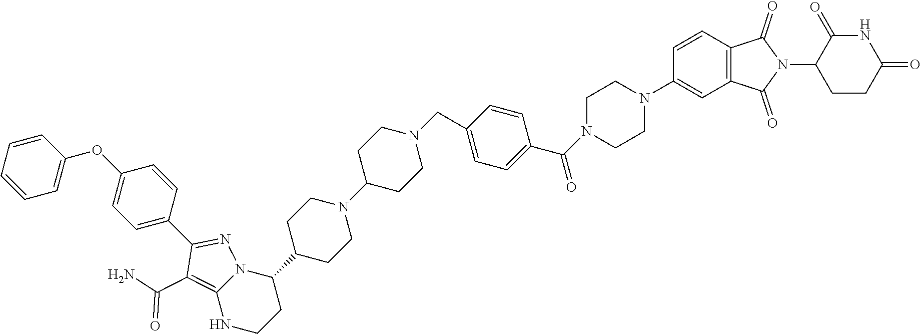 [SMILES: NC(=O)c1c(-c2ccc(Oc3ccccc3)cc2)nn2c1NCC[C@H]2C1CCN(C2CCN(Cc3ccc(C(=O)N4CCN(c5ccc6c(c5)C(=O)N(C5CCC(=O)NC5=O)C6=O)CC4)cc3)CC2)CC1]

The titled compound was synthesized in the procedures similar to Example 151. ¹H NMR (400 MHz, DMSO) δ_{H} 10.82 (s, 1H), 7.69 (d, J=8.6 Hz, 1H), 7.50 (d, J=7.4 Hz, 2H), 7.42 (d, J=13.1 Hz, 6H), 7.32 (s, 1H), 7.23 (d, J=8.3 Hz, 1H), 7.16 (s, 1H), 7.06 (d, J=7.9 Hz, 4H), 6.63 (s, 1H), 5.91 (d, J=12.5 Hz, 2H), 5.04 (s, 1H), 4.08 (s, 1H), 3.64 (s, 4H), 3.53 (s, 4H), 3.45 (s, 3H), 3.34 (s, 5H), 2.67-2.55 (m, 2H), 2.31 (s, 3H), 2.02 (s, 7H), 1.74 (d, J=27.1 Hz, 6H); [M+H]⁺=958.9.

### Example 151: (7S)-7-(1-(3-(4-(4-(2-(2, 6-dioxopiperidin-3-yl)-1, 3-dioxoisoindolin-5-yl)piperazin-1-yl)cyclohexyl)propanoyl)piperidin-4-yl)-2-(4-phenoxyphenyl)-4, 5, 6, 7-tetrahydropyrazolo[1,5-a] pyrimidine-3-carboxamide

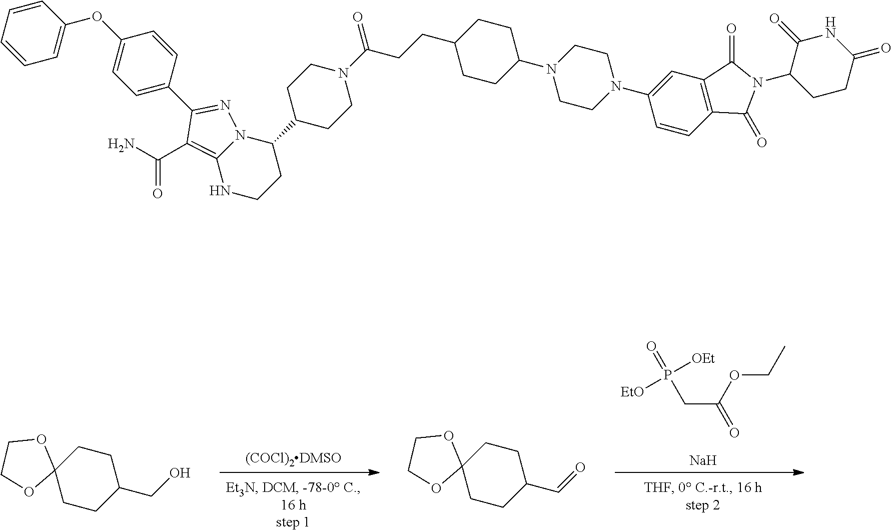

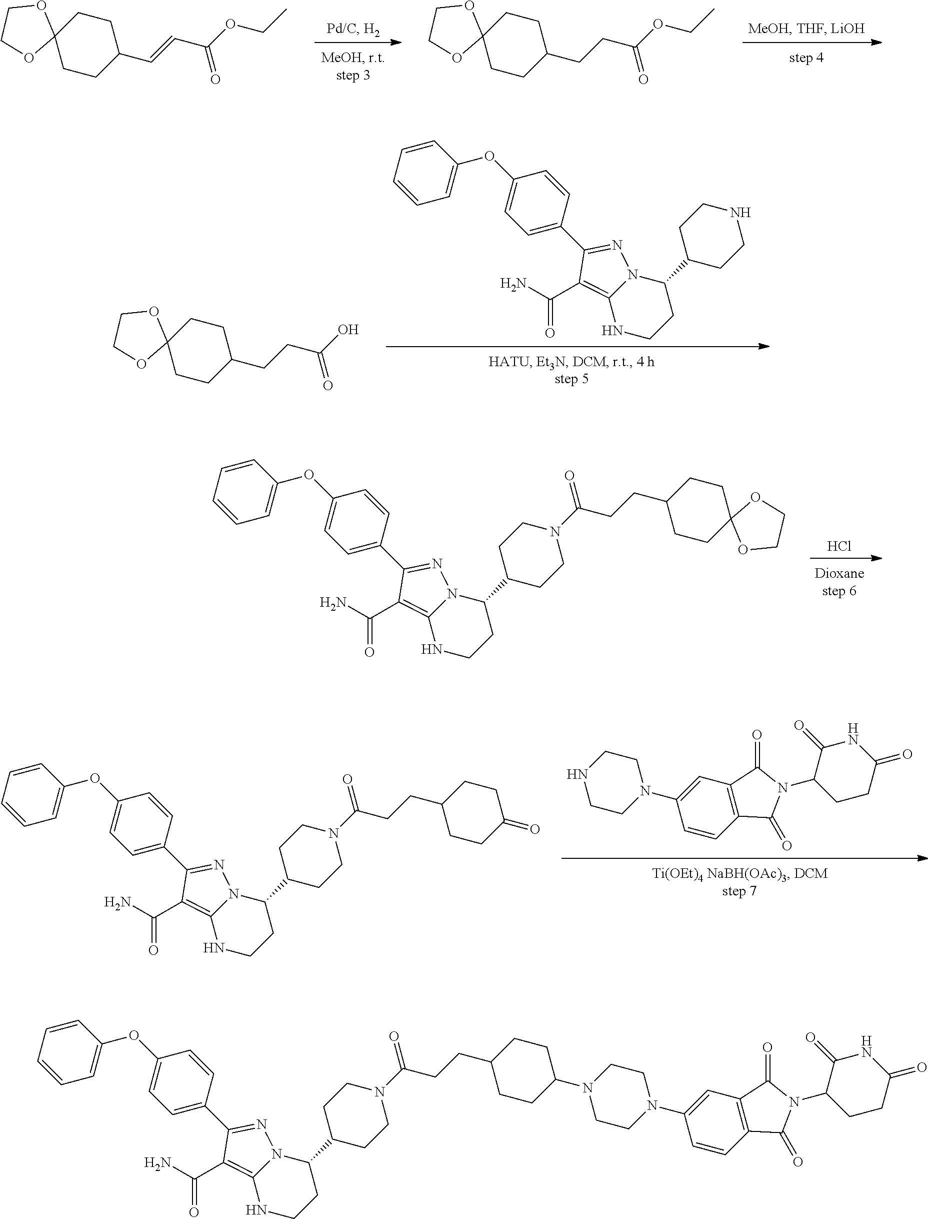

### Step 1: 1, 4-dioxaspiro[4.5]decane-8-carbaldehyde

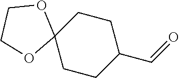 [SMILES: O=CC1CCC2(CC1)OCCO2]

To a solution of oxalyl chloride (1.91 g, 15 mmol) dissolved in DCM (20 mL) were added dropwise DMSO (2.34 g, 30 mmol) in DCM (20 mL) at −78° C. for 15 min. The mixture was stirred at −78° C. for 15 min. Then, (1, 4-dioxaspiro[4.5]decan-8-yl)methanol (1.72 g, 10 mmol) in DCM (20 mL) was added to the reaction at −78° C. for 15 min. After 20 min, Et₃N (5.60 g, 55 mmol) in DCM (40 mL) was added to the mixture at −78° C. for 15 min. The mixture was stirred at −78° C. for 2 h. The mixture was warmed to R.T. and stirred overnight. The solution was washed with water 100 mL and extracted with DCM (150 mL×3). The organic layer was combined, dried over Na₂SO₄, filtered and concentrated to dryness. The residue was purified with silica gel column chromatography (PE:EA=10/1 to 4/1) to afford 1, 4-dioxaspiro[4.5]decane-8-carbaldehyde (1.2 g, 70%). ¹H NMR (400 MHz, CDCl₃) δ_{H} 9.65 (d, J=1.1 Hz, 1H), 4.00-3.90 (m, 4H), 2.30-2.22 (m, 1H), 2.00-1.89 (m, 2H), 1.81-1.68 (m, 4H), 1.65-1.54 (m, 2H).

### Step 2: Ethyl 3-(1, 4-dioxaspiro[4.5]decan-8-yl)acrylate

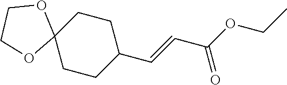 [SMILES: CCOC(=O)/C=C/C1CCC2(CC1)OCCO2]

To a mixture of ethyl 2-(diethoxyphosphoryl)acetate (806 mg, 3.60 mmol) dissolved in THF (10 mL) were added NaH (144 mg, 3.60 mmol) at 0° C. The mixture was stirred at 0° C. for 30 min. 1, 4-dioxaspiro[4.5]decane-8-carbaldehyde (510 mg, 3.00 mmol) in THF (5 mL) was added to the reaction at 0° C. The mixture was stirred at R.T. for 16 h. The mixture was washed water and extracted with EA (30 mL×3). The organic layers were combined, dried over Na₂SO₄ and concentrated. The residue was purified with silica gel column chromatography (PE:EA=10/1 to 5/1) to obtain ethyl 3-(1, 4-dioxaspiro[4.5]decan-8-yl)acrylate (648 mg, 90%). ¹H NMR (400 MHz, CDCl₃) δ_{H} 6.92 (dd, J=15.8, 6.9 Hz, 1H), 5.84-5.77 (m, 1H), 4.18 (q, J=7.1 Hz, 2H), 3.97-3.92 (m, 4H), 2.24-2.14 (m, 1H), 1.79 (d, J=9.8 Hz, 4H), 1.62-1.48 (m, 4H), 1.28 (dd, J=9.1, 5.2 Hz, 1H).

### Step 3: ethyl 3-(1, 4-dioxaspiro[4.5]decan-8-yl)propanoate

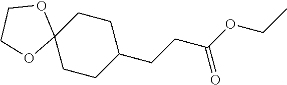 [SMILES: CCOC(=O)CCC1CCC2(CC1)OCCO2]

To a solution of ethyl 3-(1, 4-dioxaspiro[4.5]decan-8-yl)acrylate (648 mg, 2.7 mmol) dissolved in MeOH (20 ml) was added Pd/C (80 mg, w.t. 10%). And the mixture was stirred at room temperature under one hydrogen balloon overnight. Then the mixture was filtered and concentrated to afford ethyl 3-(1, 4-dioxaspiro[4.5]decan-8-yl)propanoate (640 mg, 98%). ¹H NMR (400 MHz, CDCl₃) δ_{H} 4.12 (q, J=7.1 Hz, 2H), 3.93 (s, 4H), 2.37-2.28 (m, 2H), 1.80-1.67 (m, 4H), 1.62-1.46 (m, 4H), 1.25 (t, J=7.1 Hz, 4H).

### Step 4: 3-(1, 4-dioxaspiro[4,5]decan-8-yl)propanoic Acid

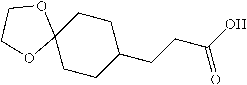 [SMILES: O=C(O)CCC1CCC2(CC1)OCCO2]

To a mixture of ethyl 3-(1, 4-dioxaspiro[4.5]decan-8-yl)propanoate (640 mg, 2.6 mmol) dissolved in MeOH (4 mL)/water (4 mL)/THF (4 mL) was added LiOH (312 g, 13 mmol). The mixture was stirred at room temperature for 16 hours. The solvent was concentrated and extracted with EA (20 mL×3) to remove the impurities. The water layer was concentrated to 5 ml. The pH value of the water layer was adjusted to 3-5 with 1N HCl. Then the mixture was added to water (10 ml) and extracted with EA (40 ml×3). The organic layer was dried over Na₂SO₄, filtered and concentrated to give 3-(1, 4-dioxaspiro[4.5]decan-8-yl)propanoic acid (529 mg, 95%). [M+H]⁺=215.0.

### Step 5: (S)-7-(1-(3-(1, 4-dioxaspiro[4.5]decan-8-yl)propanoyl)piperidin-4-yl)-2-(4-phenoxyphenyl)-4, 5, 6, 7-tetrahydropyrazolo[1,5-a]pyrimidine-3-carboxamide

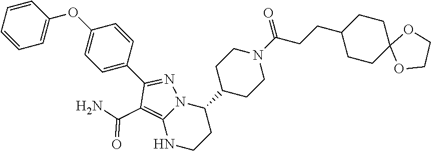 [SMILES: NC(=O)c1c(-c2ccc(Oc3ccccc3)cc2)nn2c1NCC[C@H]2C1CCN(C(=O)CCC2CCC3(CC2)OCCO3)CC1]

To a solution of 3-(1, 4-dioxaspiro[4.5]decan-8-yl)propanoic acid (214 mg, 1 mmol) in DCM (15 mL) was added HATU (456 mg, 1.20 mmol). The mixture was stirred at R.T. for 10 min. Then, Et₃N (303 mg, 3.00 mmol) and (S)-2-(4-phenoxyphenyl)-7-(piperidin-4-yl)-4, 5, 6, 7-tetrahydropyrazolo[1,5-a]pyrimidine-3-carboxamide (418 mg, 1.00 mmol) were added to the mixture. The reaction was stirred at R.T. for 2 h. The mixture was washed with water (10 mL) and extracted with DCM (10 mL×3). The organic layers were combined and dried over Na₂SO₄. The mixture was filtered and the filtrate was concentrated in vacuum. The residue was purified with pre-TLC (DCM:MeOH=100/1 to 20/1) to afford (S)-7-(1-(3-(1, 4-dioxaspiro[4.5]decan-8-yl)propanoyl)piperidin-4-yl)-2-(4-phenoxyphenyl)-4, 5, 6, 7-tetrahydropyrazolo[1,5-a]pyrimidine-3-carboxamide (600 mg, 97.8%). [M+H]⁺=613.9.

### Step 6: (S)-7-(1-(3-(4-oxocyclohexyl)propanoyl)piperidin-4-yl)-2-(4-phenoxyphenyl)-4, 5, 6, 7-tetrahydropyrazolo[1,5-a]pyrimidine-3-carboxamide

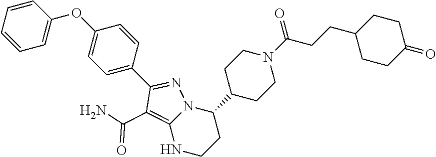 [SMILES: NC(=O)c1c(-c2ccc(Oc3ccccc3)cc2)nn2c1NCC[C@H]2C1CCN(C(=O)CCC2CCC(=O)CC2)CC1]

To a mixture of (S)-7-(1-(3-(1, 4-dioxaspiro[4.5]decan-8-yl)propanoyl)piperidin-4-yl)-2-(4-phenoxyphenyl)-4, 5, 6, 7-tetrahydropyrazolo[1,5-a]pyrimidine-3-carboxamide (600 mg, 0.98 mmol) dissolved in dioxane (5 mL) was added HCl/dioxane (2 mL, 8 mmol). The mixture was stirred at room temperature over overnight. Solvent was removed under reduced pressure. The pH value of the residue was adjusted to 8-10, and the residue was extracted with DCM (20 mL×3). The organic layers were combined and dried over Na₂SO₄. The mixture was filtered and the filtrate was concentrated in vacuum to obtain (S)-7-(1-(3-(4-oxocyclohexyl)propanoyl)piperidin-4-yl)-2-(4-phenoxyphenyl)-4, 5, 6, 7-tetrahydropyrazolo[1,5-a]pyrimidine-3-carboxamide (529 mg, 95%). [M+H]⁺=570.1.

### Step 7: (7S)-7-(1-(3-(4-(4-(2-(2, 6-dioxopiperidin-3-yl)-1, 3-dioxoisoindolin-5-yl)piperazin-1-yl)cyclohexyl)propanoyl)piperidin-4-yl)-2-(4-phenoxyphenyl)-4, 5, 6, 7-tetrahydropyrazolo[1, 5-a]pyrimidine-3-carboxamide

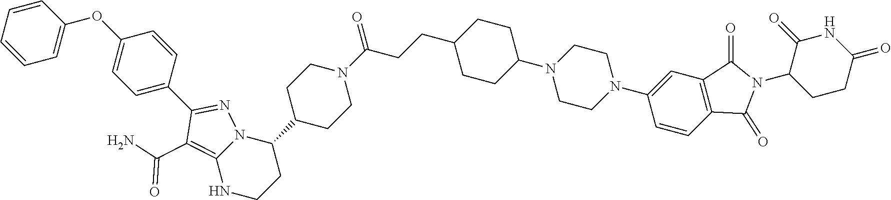 [SMILES: NC(=O)c1c(-c2ccc(Oc3ccccc3)cc2)nn2c1NCC[C@H]2C1CCN(C(=O)CCC2CCC(N3CCN(c4ccc5c(c4)C(=O)N(C4CCC(=O)NC4=O)C5=O)CC3)CC2)CC1]

To a solution of the compound (S)-7-(1-(3-(4-oxocyclohexyl)propanoyl)piperidin-4-yl)-2-(4-phenoxyphenyl)-4, 5, 6, 7-tetrahydropyrazolo[1,5-a]pyrimidine-3-carboxamide (235 mg, 0.5 mmol) and 2-(2, 6-dioxopiperidin-3-yl)-5-(piperazin-1-yl)isoindoline-1, 3-dione (138 mg, 0.5 mmol) dissolved in DCM (10 ml) were added tetraethyl titanate (456 mg, 2 mmol). The mixture was stirred at room temperature overnight. Then, sodium triacetoxyborohydride (424 mg, 2 mmol, 4 eq) was added to the reaction. The solution was quenched with Na₂SO₄.10 H₂O and extracted with DCM (50 mL). The mixture was filtered and the filtrate was concentrated in vacuum. The residue was purified Prep-HPLC to afford (7S)-7-(1-(3-(4-(4-(2-(2, 6-dioxopiperidin-3-yl)-1, 3-dioxoisoindolin-5-yl)piperazin-1-yl)cyclohexyl)propanoyl)piperidin-4-yl)-2-(4-phenoxyphenyl)-4, 5, 6, 7-tetrahydropyrazolo[1,5-a]pyrimidine-3-carboxamide (90 mg, 20%). [M+H]⁺=896.0. ¹H NMR (400 MHz, DMSO) δ_{H} 11.08 (s, 1H), 7.67 (d, J=8.3 Hz, 1H), 7.50 (d, J=8.5 Hz, 2H), 7.41 (dd, J=11.1, 4.7 Hz, 2H), 7.32 (s, 1H), 7.23 (d, J=9.1 Hz, 1H), 7.17 (t, J=7.4 Hz, 1H), 7.06 (dd, J=12.5, 5.4 Hz, 4H), 6.68 (s, 1H), 5.07 (dd, J=12.9, 5.3 Hz, 1H), 4.45 (d, J=12.0 Hz, 1H), 4.05-3.84 (m, 2H), 3.41 (s, 4H), 3.30 (s, 2H), 2.99-2.82 (m, 2H), 2.58 (d, J=16.7 Hz, 6H), 2.47-2.37 (m, 1H), 2.35-2.20 (m, 4H), 2.03-1.85 (m, 3H), 1.83-1.58 (m, 4H), 1.54 (d, J=11.8 Hz, 1H), 1.49-1.27 (m, 8H), 1.24-1.10 (m, 2H); [M+H]⁺=895.9.

### Example 152: (7S)-7-(1-(4-(4-((2-(2, 6-dioxopiperidin-3-yl)-1, 3-dioxoisoindolin-5-yl)glycyl)piperazin-1-yl)butanoyl)piperidin-4-yl)-2-(4-phenoxyphenyl)-4, 5, 6, 7-tetrahydropyrazolo[1,5-a]pyrimidine-3-carboxamide

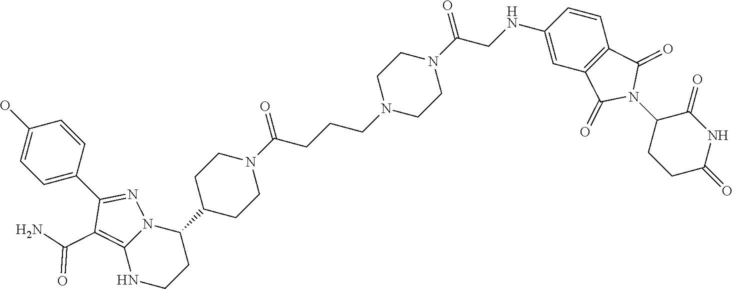 [SMILES: NC(=O)c1c(-c2ccc(O)cc2)nn2c1NCC[C@H]2C1CCN(C(=O)CCCN2CCN(C(=O)CNc3ccc4c(c3)C(=O)N(C3CCC(=O)NC3=O)C4=O)CC2)CC1]

The titled compound was synthesized in the procedures similar to Example 46. ¹H NMR (400 MHz, DMSO) δ_{H} 11.07 (s, 1H), 7.58-7.40 (m, 5H), 7.19-7.05 (m, 8H), 6.68 (s, 1H), 5.05-5.03 (m, 1H), 4.47-4.45 (m, 1H), 4.12-3.92 (m, 5H), 3.55-3.51 (m, 6H), 2.96-2.85 (m, 3H), 2.45-2.20 (m, 8H), 2.09-1.86 (m, 4H), 1.79-1.61 (m, 4H), 1.59-1.51 (m, 1H), 1.39-1.25 (m, 2H); [M+H]⁺=885.0.

### Example 153: (7S)-7-(1-(3-((4-(4-(2-(2, 6-dioxopiperidin-3-yl)-1, 3-dioxoisoindolin-5-yl)piperazin-1-yl)benzyl)(methyl)amino)propanoyl)piperidin-4-yl)-2-(4-phenoxyphenyl)-4, 5, 6, 7-tetrahydropyrazolo[1,5-a]pyrimidine-3-carboxamide

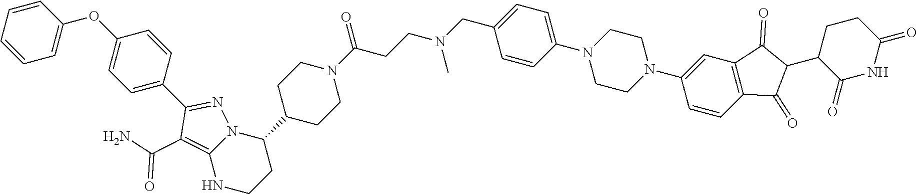 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)C(C3CCC(=O)NC3=O)C4=O)CC2)cc1]

The titled compound was synthesized in the procedures similar to Example 151. ¹H NMR (400 MHz, DMSO) δ_{H} 11.09 (s, 1H), 9.62 (s, 1H), 7.72 (d, J=8.4 Hz, 1H), 7.50 (d, J=8.4 Hz, 2H), 7.45-7.29 (m, 6H), 7.18 (d, J=7.3 Hz, 1H), 7.07 (t, J=7.7 Hz, 6H), 6.69 (s, 1H), 5.08 (dd, J=12.8, 5.2 Hz, 1H), 4.42 (d, J=11.9 Hz, 1H), 4.22 (s, 2H), 4.01 (s, 1H), 3.86 (d, J=12.1 Hz, 1H), 3.62 (s, 4H), 3.38 (s, 3H), 3.13 (s, 1H), 3.03-2.83 (m, 4H), 2.71-2.52 (m, 8H), 2.22-2.18 (m, 1H), 2.03-1.97 (m, 3H), 1.75-1.51 (m, 2H), 1.08-1.43 (m, 4H); [M+H]⁺=933.9.

### Example 154: (7S)-7-(1-(3-((2-(2, 6-dioxopiperidin-3-yl)-1, 3-dioxoisoindolin-4-yl)amino)propanoyl)piperidin-4-yl)-2-(4-phenoxyphenyl)-4, 5, 6, 7-tetrahydropyrazolo[1, 5-a]pyrimidine-3-carboxamide

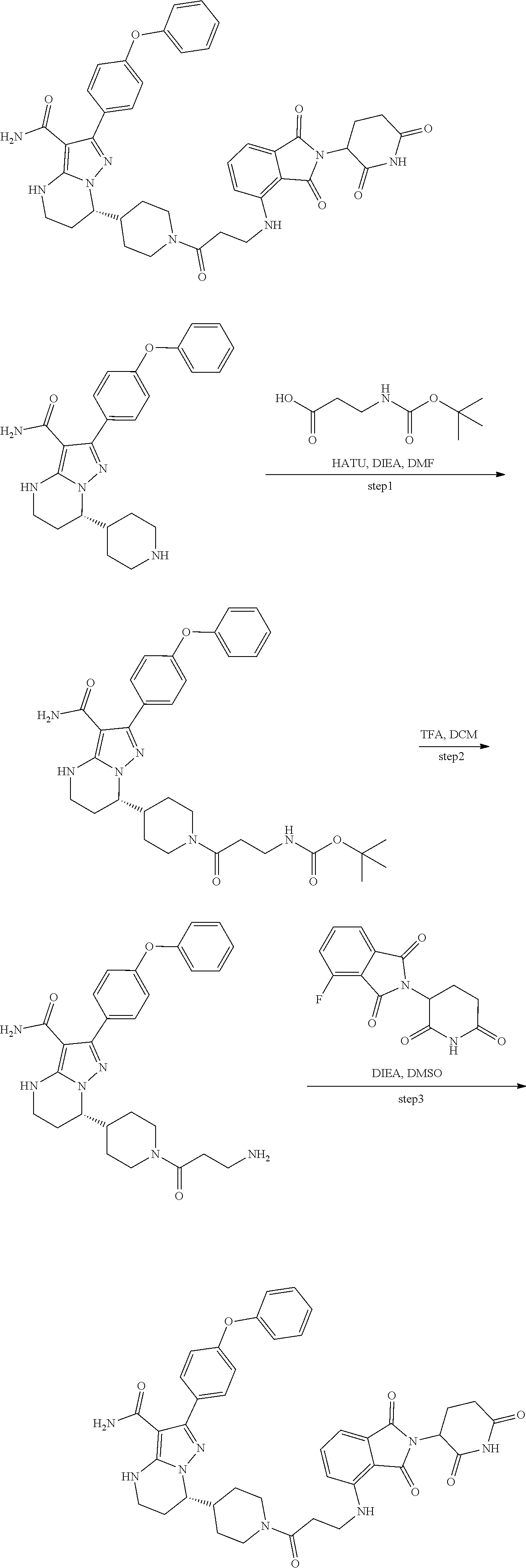

### Step 1: tert-butyl (S)-(3-(4-(3-carbamoyl-2-(4-phenoxyphenyl)-4, 5, 6, 7-tetrahydropyrazolo[1, 5-a]pyrimidin-7-yl)piperidin-1-yl)-3-oxopropyl)carbamate

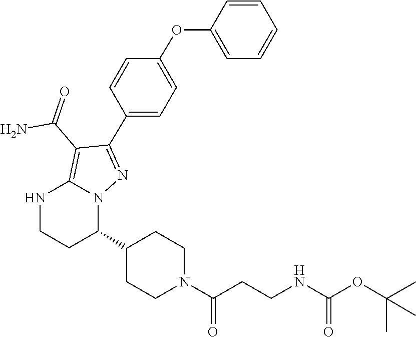 [SMILES: CC(C)(C)OC(=O)NCCC(=O)N1CCC([C@@H]2CCNc3c(C(N)=O)c(-c4ccc(Oc5ccccc5)cc4)nn32)CC1]

To a solution of the compound 3-((tert-butoxycarbonyl)amino)propanoic acid (0.476 g, 2.515 mmol) dissolved in DMF (15 ml) were added (S)-2-(4-phenoxyphenyl)-7-(piperidin-4-yl)-4, 5, 6, 7-tetrahydropyrazolo[1,5-a]pyrimidine-3-carboxamide (1.0 g, 2.395 mmol) and DIEA (1.55 g, 11.98 mmol). Then HATU (0.956 g, 2.515 mmol) was added in one portion. The mixture was stirred at room temperature overnight. After EA (150 mL) was added, the solution was washed with brine 50 ml 3 times. The organic layer was dried over Na₂SO₄, filtered and concentrated to dryness. The residue was purified with column chromatography (DCM/MeOH=10:1) to afford the product (1.4 g, crude). [M+H]⁺=589.0

### Step 2: (S)-7-(1-(3-aminopropanoyl)piperidin-4-yl)-2-(4-phenoxyphenyl)-4, 5, 6, 7-tetrahydropyrazolo[1,5-a]pyrimidine-3-carboxamide

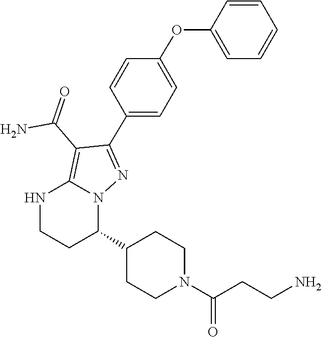 [SMILES: NCCC(=O)N1CCC([C@@H]2CCNc3c(C(N)=O)c(-c4ccc(Oc5ccccc5)cc4)nn32)CC1]

To a solution of tert-butyl (S)-(3-(4-(3-carbamoyl-2-(4-phenoxyphenyl)-4, 5, 6, 7-tetrahydropyrazolo[1,5-a]pyrimidin-7-yl)piperidin-1-yl)-3-oxopropyl)carbamate (1.4 g, crude) in DCM (10 ml), TFA (5 ml) was added. The mixture was stirred at 25° C. for 1 hour. The solution was concentrated to dryness, purified with column chromatography (DCM/MeOH=10:1, 1% ammonia water) to afford the product (0.708 g, 61%, two steps). [M+H]⁺=488.9

### Step 3: (7S)-7-(1-(3-((2-(2, 6-dioxopiperidin-3-yl)-1, 3-dioxoisoindolin-4-yl)amino) propanoyl)piperidin-4-yl)-2-(4-phenoxyphenyl)-4, 5, 6, 7-tetrahydropyrazolo[1,5-a]pyrimidine-3-carboxamide

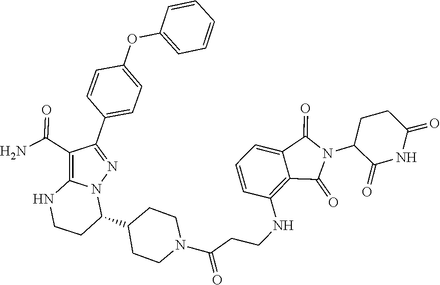 [SMILES: NC(=O)c1c(-c2ccc(Oc3ccccc3)cc2)nn2c1NCC[C@H]2C1CCN(C(=O)CCNc2cccc3c2C(=O)N(C2CCC(=O)NC2=O)C3=O)CC1]

To a solution of (S)-7-(1-(3-aminopropanoyl)piperidin-4-yl)-2-(4-phenoxyphenyl)-4, 5, 6, 7-tetrahydropyrazolo[1,5-a]pyrimidine-3-carboxamide (0.1 g, 0.205 mmol) and 2-(2, 6-dioxopiperidin-3-yl)-4-fluoroisoindoline-1, 3-dione (0.062 g, 0.225 mmol) in DMSO (2 ml) was added DIEA (0.092 g, 0.716 mmol). The mixture was stirred at 110° C. overnight. The mixture was dissolved in EA (20 ml), washed with brine (10 ml*3) and concentrated to dryness. The crude residue was purified with pre-TLC (DCM/MeOH 15/1) to give the title product (72.96 mg, 48%). ¹H NMR (400 MHz, DMSO) δ_{H} 11.10 (s, 1H), 7.58 (t, J=7.9 Hz, 1H), 7.50 (d, J=8.4 Hz, 2H), 7.44-7.39 (m, 2H), 7.16 (dd, J=16.6, 8.1 Hz, 2H), 7.11-7.00 (m, 5H), 6.77 (s, 1H), 6.67 (s, 1H), 5.04 (dd, J=12.9, 5.3 Hz, 1H), 4.47 (s, 1H), 4.03-3.85 (m, 2H), 3.53 (d, J=6.4 Hz, 2H), 3.31 (m, 3H), 3.00-2.82 (m, 2H), 2.74-2.55 (m, 3H), 2.45 (d, J=13.4 Hz, 1H), 2.23 (s, 1H), 1.95 (d, J=33.2 Hz, 3H), 1.68 (s, 1H), 1.50 (s, 1H), 1.34-1.05 (m, 2H); [M+H]⁺=744.8.

### Example 155: (S)-7-(1-(3-(((1-(4-(2, 4-dioxotetrahydropyrimidin-1(2H)-yl)benzoyl) piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

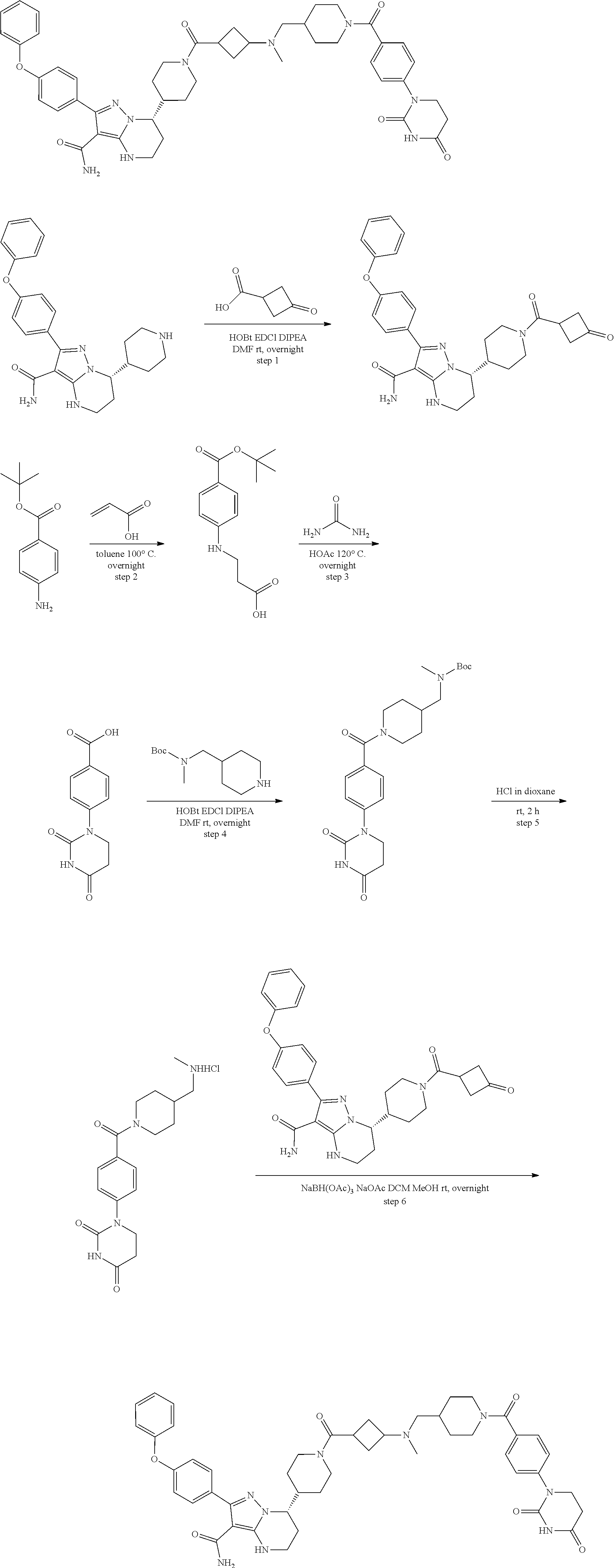

### Step 1: (S)-7-(1-(3-oxocyclobutane-1-carbonyl)piperidin-4-yl)-2-(4-phenoxyphenyl)-4, 5, 6, 7-tetrahydropyrazolo[1,5-a]pyrimidine-3-carboxamide

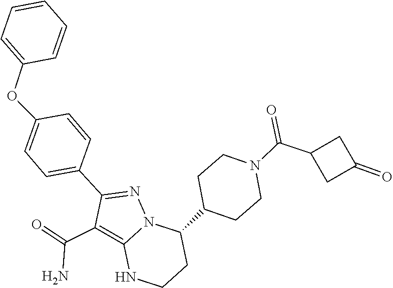 [SMILES: NC(=O)c1c(-c2ccc(Oc3ccccc3)cc2)nn2c1NCC[C@H]2C1CCN(C(=O)C2CC(=O)C2)CC1]

A mixture of 3-oxocyclobutane-1-carboxylic acid (0.3 g, 0.0026 mol), HOBt (0.518 g, 0.0038 mol), EDCI (0.722 g, 0.0038 mol) and DIPEA (0.744 g, 0.006 mol,) in DMF (10 mL) was stirred at room temperature for 5 min. (S)-2-(4-phenoxyphenyl)-7-(piperidin-4-yl)-4, 5, 6, 7-tetrahydropyrazolo[1,5-a]pyrimidine-3-carboxamide (1.0 g, 0.0024 mol) was added. The mixture was stirred at room temperature overnight. After the reaction was completed as indicated by LCMS, the reaction mixture was extracted with EA (100 mL*3) and washed with brine (100 mL*2). The combined organic solution was dried over Na₂SO₄ and concentrated in vacuo. The residue was purified with silica gel chromatography (DCM:MeOH=90:10) to give the target product (1.02 g, 83%). [M+H]⁺=514.0.

### Step 2: 3((4-(tert-butoxycarbonyl)phenyl)amino)propanoic acid

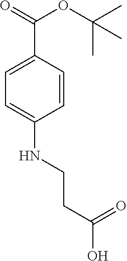 [SMILES: CC(C)(C)OC(=O)c1ccc(NCCC(=O)O)cc1]

A mixture of tert-butyl 4-aminobenzoate (5 g, 0.0259 mol) and acrylic acid (2.0 g, 0.0285 mol) in toluene (50 mL) was stirred at 100° C. overnight. After the reaction was completed as determined by LCMS, the mixture was cooled to room temperature, then filtered with PE:EA=20:1 (200 mL). The filtrate was purified to give the target product (3.4 g, crude). [M+H]⁺=266.0.

### Step 3: 4-(2, 4-dioxotetrahydropyrimidin-1(2H)-yl)benzoic Acid

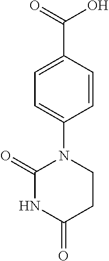 [SMILES: O=C1CCN(c2ccc(C(=O)O)cc2)C(=O)N1]

A mixture of 3-((4-(tert-butoxycarbonyl)phenyl)amino)propanoic acid (3.2 g, 0.012 mol), and urea (1.8 g, 0.030 mol) in HOAc (30 mL) was stirred at 120° C. overnight. After the reaction was completed determined by LCMS, the mixture was cooled to room temperature, and concentrated in vacuo. EA (200 mL) was added into the residue and stirred at R. T. for 30 min, then filtered to collect the solid to give the target product (3.7 g, crude), which was used in next step without further purification. [M+H]⁺=234.9.

### Step 4: tert-butyl ((1-(4-(2, 4-dioxotetrahydropyrimidin-1(2H)-yl)benzoyl)piperidin-4-yl)methyl)(methyl)carbamate

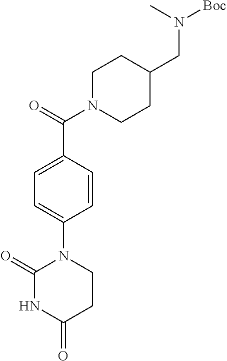 [SMILES: CN(CC1CCN(C(=O)c2ccc(N3CCC(=O)NC3=O)cc2)CC1)C(=O)OC(C)(C)C]

A mixture of 4-(2, 4-dioxotetrahydropyrimidin-1(2H)-yl)benzoic acid (1 g crude, 0.0043 mol,), HOBt (0.97 g, 0.00731 mol), EDCI (1.39 g, 0.00731 mol) and DIPEA (1.39 g, 0.0108 mol) in DMF (20 mL) was stirred at room temperature for 5 min. Then tert-butyl methyl(piperidin-4-ylmethyl)carbamate (1.08 g, 0.00473 mol) was added. The mixture was stirred at room temperature overnight. After the reaction was completed as determined by LCMS, the reaction mixture was extracted with DCM (50 mL*3), washed with water (100 mL*2). The combined organic solution was dried over Na₂SO₄ and concentrated in vacuo. The residue was purified with silica gel chromatography (DCM:MeOH=90:10) to give target product (626 mg, 32%). [M+H-tBu]⁺=389.0.

### Step 5: 1-(4-(4-((methylamino)methyl)piperidine-1-carbonyl)phenyl)dihydropyrimidine-2, 4(1H, 3H)-dione hydrochloride

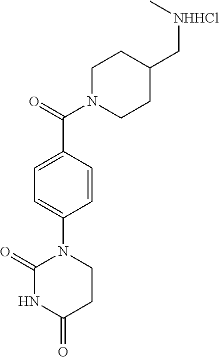

Tert-butyl ((1-(4-(2, 4-dioxotetrahydropyrimidin-1(2H)-yl)benzoyl)piperidin-4-yl)methyl)(methyl)carbamate (476 mg, 1.072 mol) was dissolved in 4 M HCl in dioxane (20 mL). Then the mixture was stirred at room temperature for 2 h. After the reaction was completed as determined by LCMS, the reaction mixture was concentrated in vacuo to afford desired product (467 mg, crude), which was used in the next step without further purification. [M+H]⁺=344.0.

### Step 6: (S)-7-(1-(3-(((1-(4-(2, 4-dioxotetrahydropyrimidin-1(2H)-yl)benzoyl)piperidin-4-yl)methyl)(methyl)amino)cyclobutane-1-carbonyl)piperidin-4-yl)-2-(4-phenoxyphenyl)-4, 5, 6, 7-tetrahydropyrazolo[1,5-a]pyrimidine-3-carboxamide

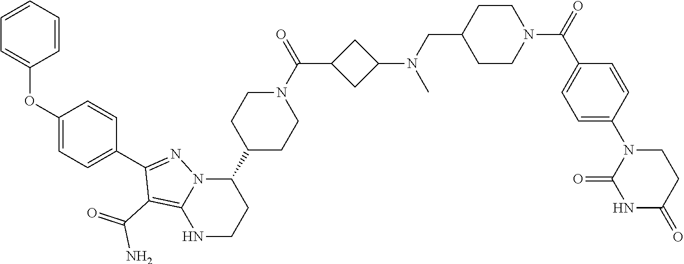 [SMILES: CN(CC1CCN(C(=O)c2ccc(N3CCC(=O)NC3=O)cc2)CC1)C1CC(C(=O)N2CCC([C@@H]3CCNc4c(C(N)=O)c(-c5ccc(Oc6ccccc6)cc5)nn43)CC2)C1]

A mixture of 1-(4-(4-((methylamino)methyl)piperidine-1-carbonyl)phenyl)dihydropyrimidine-2, 4(1H, 3H)-dione hydrochloride (100 mg, 0.263 mmol) and NaOAc (49 mg, 0.597 mmol) in DCM:MeOH=1:1 (10 mL) was stirred at room temperature for 30 min. (S)-7-(1-(3-oxocyclobutane-1-carbonyl)piperidin-4-yl)-2-(4-phenoxyphenyl)-4, 5, 6, 7-tetrahydropyrazolo[1,5-a]pyrimidine-3-carboxamide (123.8 mg, 0.238 mmol) was added. The mixture was stirred at room temperature overnight. Then NaBH(OAc)₃ (202 mg, 0.952 mmol) was added. The mixture was stirred at room temperature for 2.5 h. After the reaction was completed as determined by LCMS, the reaction mixture was quenched with 10 mL water and diluted with DCM (100 mL). The mixture was filtered, and the filtrate was concentrated in vacuo. The residue was purified with C18 chromatography (H₂O: MeCN=50:50) to give the target product (30 mg, 14%).

¹H NMR (400 MHz, DMSO) δ_{H} 10.43 (s, 1H), 7.50 (d, J=8.0 Hz, 2H), 7.45-7.35 (m, 6H), 7.17 (t, J=8.0 Hz, 1H), 7.07 (dd, J=12.0, 8.0 Hz, 4H), 6.67 (d, J=8.0 Hz, 1H), 4.53-4.34 (m, 2H), 4.05-3.96 (m, 1H), 3.86-3.73 (m, 3H), 3.60 (s, 1H), 3.30 (s, 2H), 3.11-2.81 (m, 4H), 2.77-2.56 (m, 4H), 2.48-2.38 (m, 1H), 2.25-2.12 (m, 3H), 1.98 (s, 6H), 1.90-1.80 (m, 2H), 1.77-1.61 (m, 4H), 1.51 (t, J=12.0 Hz, 1H), 1.32-1.10 (m, 2H), 1.09-0.95 (m, 2H); [M+H]⁺=842.8.

### Example 156: (7S)-7-(1-(3-(4-(4-(2-(2, 6-dioxopiperidin-3-yl)-1, 3-dioxoisoindolin-5-yl)piperazin-1-yl)piperidin-1-yl)propanoyl)piperidin-4-yl)-2-(4-phenoxyphenyl)-4, 5, 6, 7-tetrahydropyrazolo[1,5-a]pyrimidine-3-carboxamide

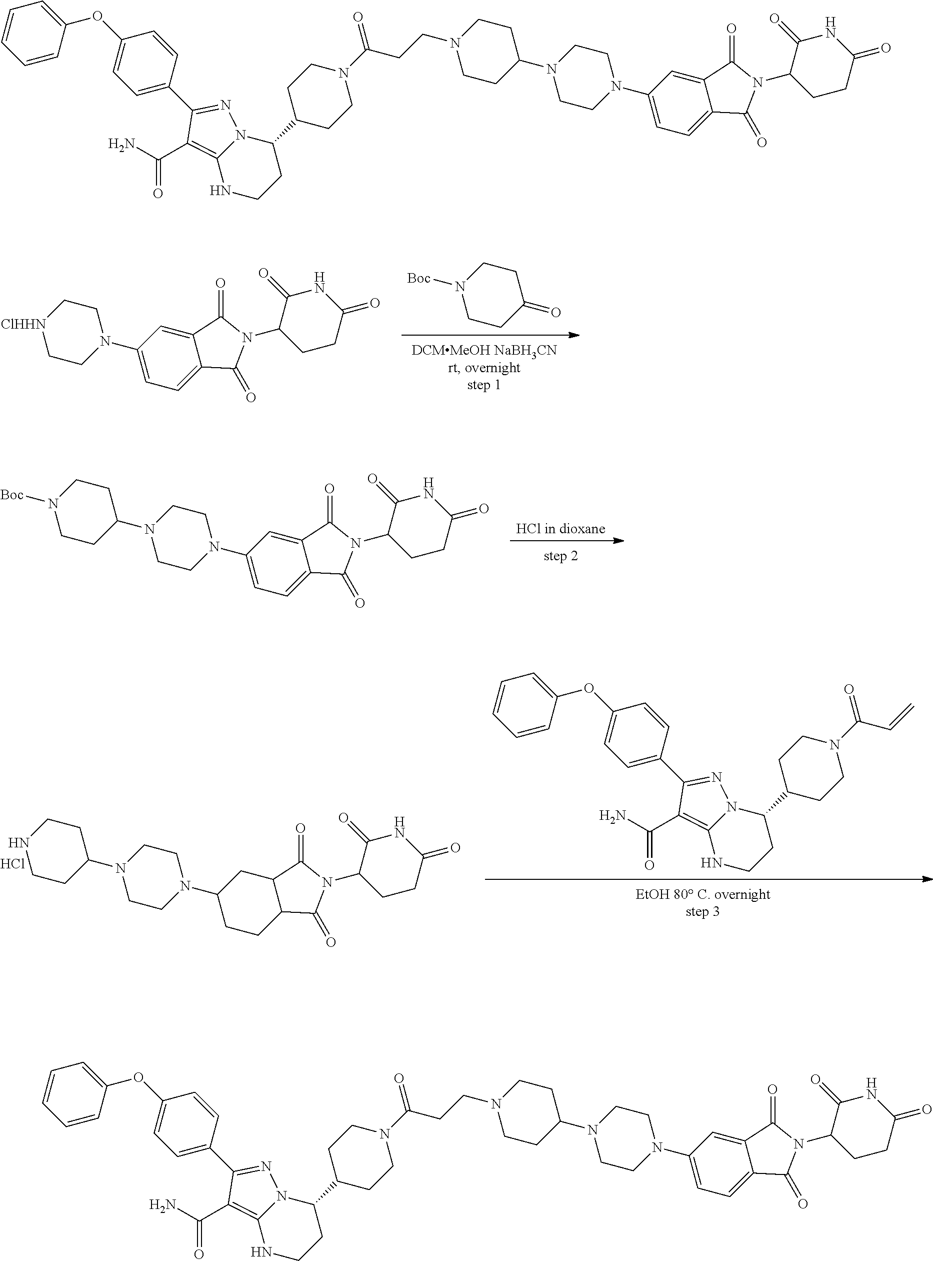

### Step 1: tert-butyl 4-(4-(2-(2, 6-dioxopiperidin-3-yl)-1, 3-dioxoisoindolin-5-yl)piperazin-1-yl)piperidine-1-carboxylate

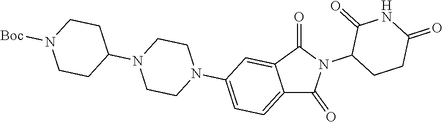 [SMILES: CC(C)(C)OC(=O)N1CCC(N2CCN(c3ccc4c(c3)C(=O)N(C3CCC(=O)NC3=O)C4=O)CC2)CC1]

To a solution of 2-(2, 6-dioxopiperidin-3-yl)-5-(piperazin-1-yl)isoindoline-1, 3-dione hydrochloride (500 mg, 1.46 mmol) in DCM:MeOH=1:1 (10 mL), NaOAc (478 mg, 5.83 mmol) was added. The mixture was stirred at room temperature for 30 min. Tert-butyl 4-oxopiperidine-1-carboxylate (290 mg, 1.46 mmol) was added. The mixture was stirred at room temperature for 4 h. Then NaBH₃CN (183 mg, 2.91 mmol) was added. The mixture was stirred at room temperature overnight. After the reaction was completed as determined by LCMS, the reaction mixture was quenched with 10 mL water and diluted with EA (100 mL). The mixture was filtered, and the filtrate was extracted with EA (50 mL*2), washed with brine, dried over Na₂SO₄, and concentrated in vacuo. The residue was purified with silica gel column chromatography (DCM:MeOH=95:5) to give target product (300 mg, 43%). [M+H]⁺=526.0.

### Step 2: 2-(2, 6-dioxopiperidin-3-yl)-5-(4-(piperidin-4-yl)piperazin-1-yl)isoindoline-1, 3-dione hydrochloride

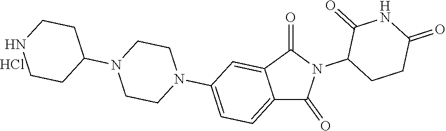 [SMILES: Cl ; O=C1CCC(N2C(=O)c3ccc(N4CCN(C5CCNCC5)CC4)cc3C2=O)C(=O)N1]

Tert-butyl 4-(4-(2-(2, 6-dioxopiperidin-3-yl)-1, 3-dioxoisoindolin-5-yl)piperazin-1-yl)piperidine-1-carboxylate (231 mg, 0.44 mol) was dissolved in 4 M HCl in dioxane (10 mL). Then the mixture was stirred at room temperature for 3 h. After the reaction was completed as determined by LCMS, the reaction mixture was concentrated in vacuo to afford desired product (241 mg, crude), which was used in the next step without further purification. [M+H]⁺=426.0.

### Step 3: (7S)-7-(1-(3-(4-(4-(2-(2, 6-dioxopiperidin-3-yl)-1, 3-dioxoisoindolin-5-yl) piperazin-1-yl)piperidin-1-yl)propanoyl)piperidin-4-yl)-2-(4-phenoxyphenyl)-4, 5, 6, 7-tetrahydropyrazolo[1, 5-a]pyrimidine-3-carboxamide

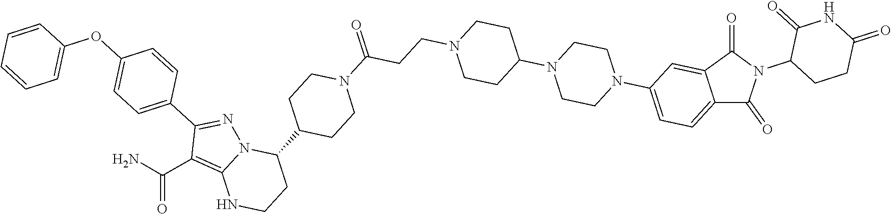 [SMILES: NC(=O)c1c(-c2ccc(Oc3ccccc3)cc2)nn2c1NCC[C@H]2C1CCN(C(=O)CCN2CCC(N3CCN(c4ccc5c(c4)C(=O)N(C4CCC(=O)NC4=O)C5=O)CC3)CC2)CC1]

A mixture of 2-(2, 6-dioxopiperidin-3-yl)-5-(4-(piperidin-4-yl)piperazin-1-yl)isoindoline-1, 3-dione hydrochloride (108 mg, 0.234 mmol), (S)-7-(1-acryloylpiperidin-4-yl)-2-(4-phenoxyphenyl)-4, 5, 6, 7-tetrahydropyrazolo[1,5-a] pyrimidine-3-carboxamide (100 mg, 0.213 mmol) and DIPEA (360 mg, 0.277 mmol) in EtOH (10 mL) was stirred at 80° C. overnight. After the reaction was completed as determined by LCMS, the reaction mixture was concentrated in vacuo. The residue was purified with silica gel chromatography (DCM:MeOH=10:1) to give crude product, which was further purified with pre-TLC (DCM:MeOH=12:1) to afford desired product (32 mg, 15%). ¹H NMR (400 MHz, DMSO) δ_{H} 11.09 (s, 1H), 9.40 (s, 1H), 7.69 (d, J=8.0 Hz, 1H), 7.50 (d, J=8.0 Hz, 2H), 7.42 (t, J=8.0 Hz, 2H), 7.35 (s, 1H), 7.26 (d, J=8.0 Hz, 1H), 7.18 (t, J=8.0 Hz, 1H), 7.07 (t, J=8.0 Hz, 4H), 6.70 (s, 1H), 5.07 (dd, J=12.0, 4.0 Hz, 1H), 4.44 (d, J=12.0 Hz, 1H), 4.09-3.80 (m, 2H), 3.61-3.39 (m, 6H), 3.23 (s, 3H), 3.06-2.73 (m, 7H), 2.70-2.53 (m, 8H), 2.30-2.16 (m, 1H), 2.10-1.86 (m, 6H), 1.78-1.63 (m, 3H), 1.58 (s, 1H), 1.40-1.27 (m, 1H), 1.22-1.09 (m, 1H). [M+H]⁺=897.9.

### Example 157: (7S)-7-(1-((E)-1-((2-(2, 6-dioxopiperidin-3-yl)-1-oxoisoindolin-4-yl)amino)-12-methyl-3, 6, 9-trioxa-12-azahexadec-14-en-16-oyl)piperidin-4-yl)-2-(4-phenoxyphenyl)-4, 5, 6, 7-tetrahydropyrazolo[1,5-a]pyrimidine-3-carboxamide

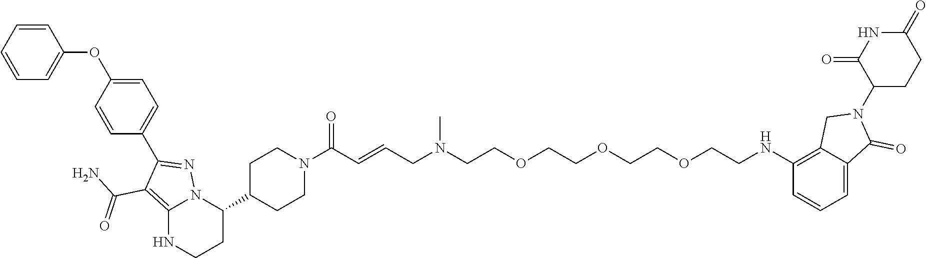 [SMILES: CN(C/C=C/C(=O)N1CCC([C@@H]2CCNc3c(C(N)=O)c(-c4ccc(Oc5ccccc5)cc4)nn32)CC1)CCOCCOCCOCCNc1cccc2c1CN(C1CCC(=O)NC1=O)C2=O]

The titled compound was synthesized in the procedures similar to Example 46. ¹H NMR (400 MHz, DMSO) δ_{H} 11.01 (s, 1H), 7.50 (d, J=8.8 Hz, 2H), 7.45-7.38 (m, 3H), 7.28 (t, J=8.0 Hz, 1H), 7.17 (t, J=7.2 Hz, 2H), 7.11-7.03 (m, 6H), 6.94 (d, J=7.6 Hz, 1H), 6.79 (d, J=8.4 Hz, 1H), 6.67 (s, 1H), 6.55 (d, J=15.6 Hz, 2H), 5.58 (s, 1H), 5.11 (dd, J=13.2, 5.2 Hz, 2H), 4.53-4.42 (m, 1H), 4.24-4.00 (m, 5H), 3.59-3.52 (m, 10H), 3.30 (s, 6H), 3.10-2.83 (m, 5H), 2.70-2.55 (m, 3H), 2.30-2.15 (m, 6H), 2.02-1.91 (m, 2H), 1.71-1.55 (m, 2H); [M+H]⁺=931.9.

### Example 158: (7S)-7-(1-(3-(((4-((2-(2, 6-dioxopiperidin-3-yl)-1, 3-dioxoisoindolin-5-yl)amino)cyclohexyl)methyl)(methyl)amino)propanoyl)piperidin-4-yl)-2-(4-phenoxyphenyl)-4, 5, 6, 7-tetrahydropyrazolo[1,5-a]pyrimidine-3-carboxamide

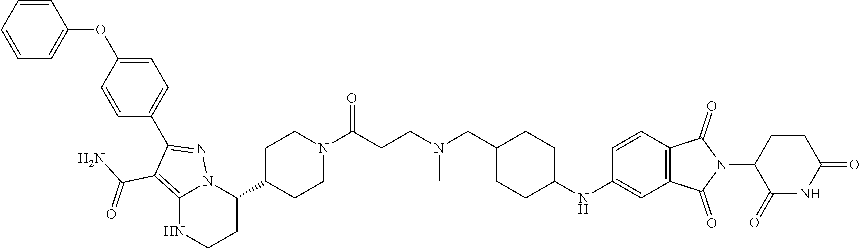 [SMILES: CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1]

The titled compound was synthesized in the procedures similar to Example 151. ¹H NMR (400 MHz, DMSO) δ₁₁ 11.08 (s, 1H), 7.65 (d, J=8.0 Hz, 1H), 7.50 (d, J=8.0 Hz, 2H), 7.44-7.40 (m, 2H), 7.30 (s, 1H), 7.23-7.15 (m, 2H), 7.09-7.05 (m, 4H), 6.69 (s, 1H), 5.09-5.04 (m, 1H), 4.45-4.42 (m, 1H), 4.05-3.91 (m, 4H), 3.31-3.30 (m, 2H), 2.98-2.84 (m, 6H), 2.60-2.45 (m, 6H), 2.30-2.15 (m, 3H), 2.08-1.98 (m, 3H), 1.95-1.55 (m, 7H), 1.35-1.13 (m, 5H); [M+H]⁺=871.0.

### Example 159: (7S)-7-(1-(3-(4-(2-(2, 6-dioxopiperidin-3-yl)-1, 3-dioxoisoindolin-5-yl)piperazin-1-yl)propanoyl)piperidin-4-yl)-2-(4-phenoxyphenyl)-4, 5, 6, 7-tetrahydropyrazolo[1,5-a]pyrimidine-3-carboxamide

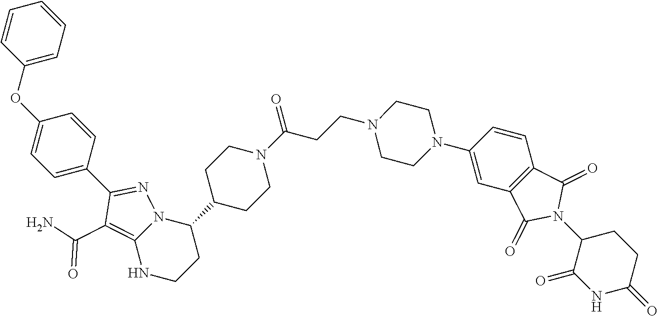 [SMILES: NC(=O)c1c(-c2ccc(Oc3ccccc3)cc2)nn2c1NCC[C@H]2C1CCN(C(=O)CCN2CCN(c3ccc4c(c3)C(=O)N(C3CCC(=O)NC3=O)C4=O)CC2)CC1]

The titled compound was synthesized in the procedures similar to Example 152. ¹H NMR (400 MHz, DMSO) δ_{H} ¹H NMR (400 MHz, DMSO) δ_{H} 11.08 (s, 1H), 7.67 (d, J=8.4 Hz, 1H), 7.50 (d, J=8.8 Hz, 2H), 7.40 (t, J=8.0 Hz, 2H), 7.34 (S, 1H), 7.25 (d, J=8.8 Hz, 2H), 7.16 (t, J=8.0 Hz, 1H), 7.04 (d, J=7.6 Hz, 1H), 6.79 (d, J=8.4 Hz, 1H), 6.67 (s, 1H), 5.08 (dd, J=13.2, 5.2 Hz, 2H), 4.46-4.44 (m, 1H), 4.02-3.95 (m, 2H), 3.30-3.42 (m, 8H), 2.93-2.83 (m, 2H), 2.59-2.50 (m, 6H), 2.30-2.22 (m, 1H), 2.02-1.93 (m, 3H), 1.74-1.52 (m, 2H), 1.35-1.15 (m, 2H.); [M+H]⁺=815.

Cell Degradation

Cell Treatment

Ramos cells are seeded at 250000 cells/well at a volume of 100 ul/well in cell culture medium [RPMI1640(Gibco Cat #22400), 10% heat-inactive FBS, 1% PS(Gibco, Cat #10378)] in Corning 96 well plate (Cat #3799). Ramos cells are treated with compounds diluted in 0.1% DMSO, dilution is done according to the following protocol: (1) make 1000x stock solution in DMSO from 10 mM by 5-fold dilution, total 7 doses were included; (2) make 10x solution in cell culture medium by transferring 1 ul 1000x stock solution into 99 ul medium; (3) 11 ul of 10× solution is added to cells and incubate for 16 h.

ELISA Assay

Cells in 96 well plate are harvested by centrifugation (1200 rpm), and wash once with ice-cold PBS. Then, 100 ul 1×cell extraction buffer is added in each well. The cells are placed on a shaker for 20 min at 4° C.; 5 ul lysate is transferred to 45 ul of 1×cell extraction buffer in ELISA plate, and 50 ul of the antibody cocktail is added in each well; seal the plate and incubate 1 hour at room temperature on a plate shaker; medium is discarded from the plate, tap the plated on a paper towel, wash 3×200 ul ELISA wash buffer; add 100 ul of TMB to each well and incubate the plate in the dark for 15 min at room temperature on a shaker; add 100 ul of STOP solution to each well; read absorbance at 450 nm. The BTK ELISA assay was performed using FastScan total BTK ELISA kit (Cell Signaling Cat #36609).

TABLE 1
Degradation result for Example 1 to Example 162

| Ex- | | |
|---|---|---|
| am- | | DC₅₀ |
| ple | Structure | (μM) |
| 1 | 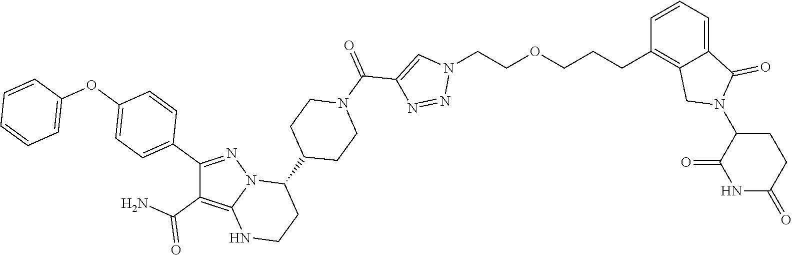 [SMILES: NC(=O)c1c(-c2ccc(Oc3ccccc3)cc2)nn2c1NCC[C@H]2C1CCN(C(=O)c2cn(CCOCCCc3cccc4c3CN(C3CCC(=O)NC3=O)C4=O)nn2)CC1] | >10 |
| 2 | 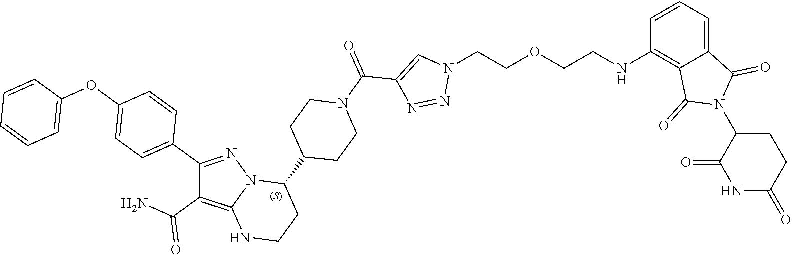 | >10 |
| 3 | 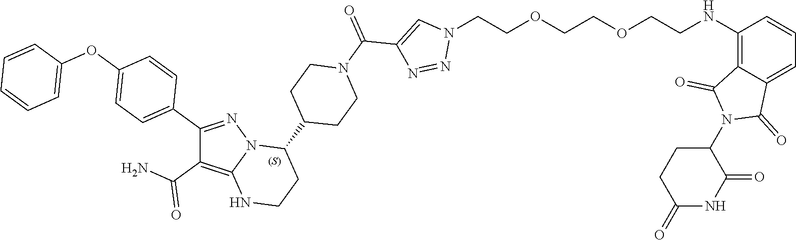 | >10 |
| 4 | 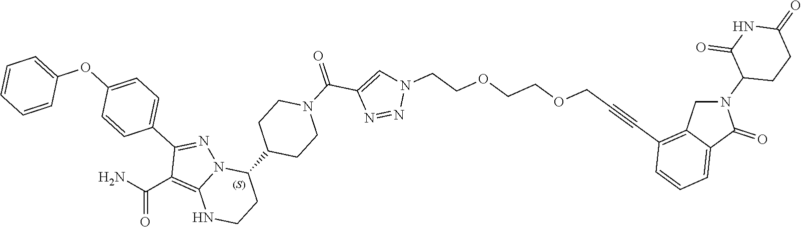 | >10 |
| 5 | 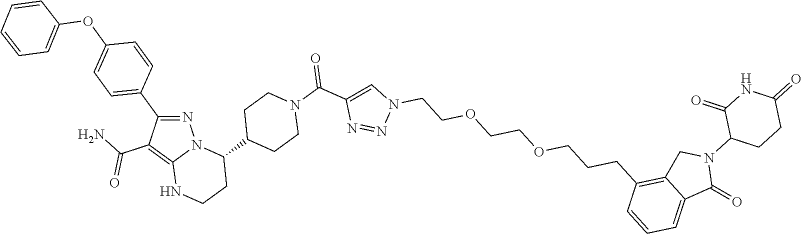 [SMILES: NC(=O)c1c(-c2ccc(Oc3ccccc3)cc2)nn2c1NCC[C@H]2C1CCN(C(=O)c2cn(CCOCCOCCCc3cccc4c3CN(C3CCC(=O)NC3=O)C4=O)nn2)CC1] | >10 |
| 6 | 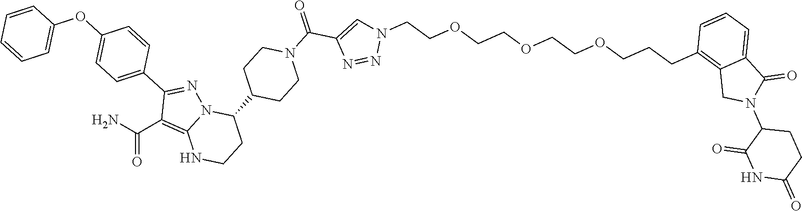 [SMILES: NC(=O)c1c(-c2ccc(Oc3ccccc3)cc2)nn2c1NCC[C@H]2C1CCN(C(=O)c2cn(CCOCCOCCOCCCc3cccc4c3CN(C3CCC(=O)NC3=O)C4=O)nn2)CC1] | 0.242 |
| 7 | 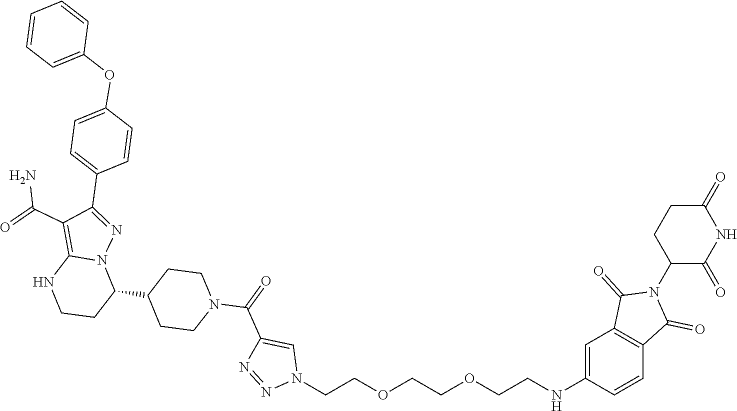 [SMILES: NC(=O)c1c(-c2ccc(Oc3ccccc3)cc2)nn2c1NCC[C@H]2C1CCN(C(=O)c2cn(CCOCCOCCNc3ccc4c(c3)C(=O)N(C3CCC(=O)NC3=O)C4=O)nn2)CC1] | >10 |
| 8 | 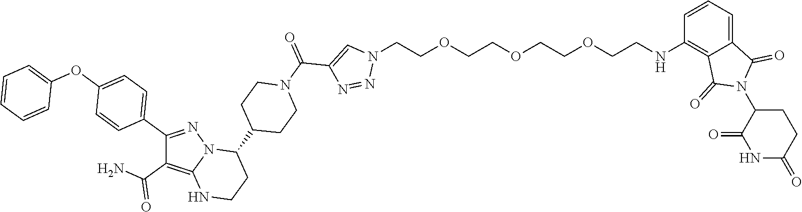 [SMILES: NC(=O)c1c(-c2ccc(Oc3ccccc3)cc2)nn2c1NCC[C@H]2C1CCN(C(=O)c2cn(CCOCCOCCOCCNc3cccc4c3C(=O)N(C3CCC(=O)NC3=O)C4=O)nn2)CC1] | 0.128 |
| 9 | 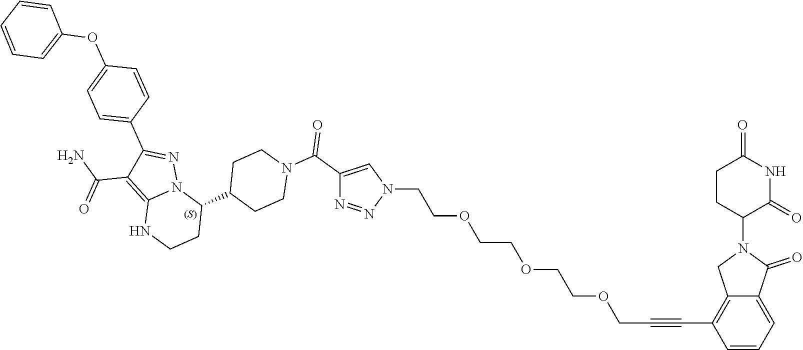 | >10 |
| 10 | 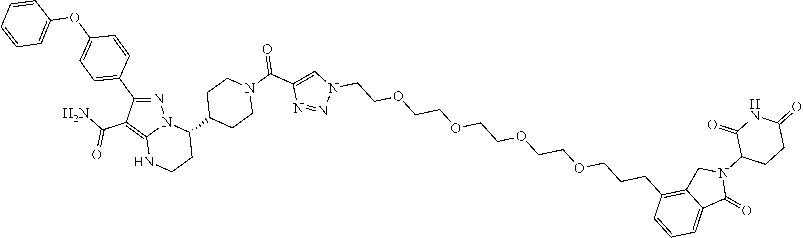 [SMILES: NC(=O)c1c(-c2ccc(Oc3ccccc3)cc2)nn2c1NCC[C@H]2C1CCN(C(=O)c2cn(CCOCCOCCOCCOCCCc3cccc4c3CN(C3CCC(=O)NC3=O)C4=O)nn2)CC1] | >10 |
| 11 | 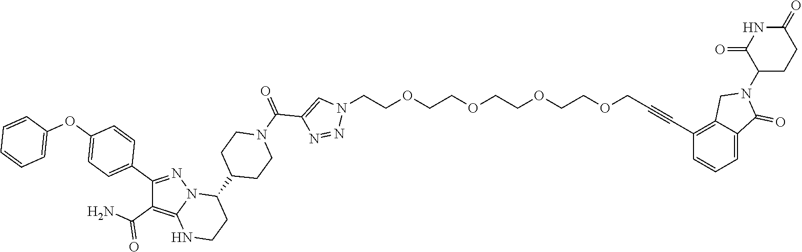 [SMILES: NC(=O)c1c(-c2ccc(Oc3ccccc3)cc2)nn2c1NCC[C@H]2C1CCN(C(=O)c2cn(CCOCCOCCOCCOCC#Cc3cccc4c3CN(C3CCC(=O)NC3=O)C4=O)nn2)CC1] | 0.176 |
| 12 | 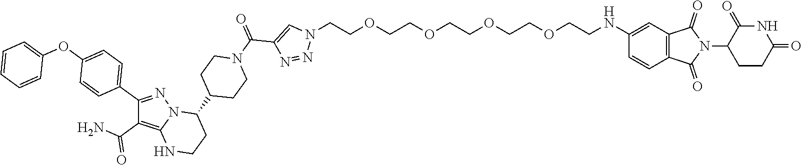 [SMILES: NC(=O)c1c(-c2ccc(Oc3ccccc3)cc2)nn2c1NCC[C@H]2C1CCN(C(=O)c2cn(CCOCCOCCOCCOCCNc3ccc4c(c3)C(=O)N(C3CCC(=O)NC3=O)C4=O)nn2)CC1] | >10 |
| 13 | 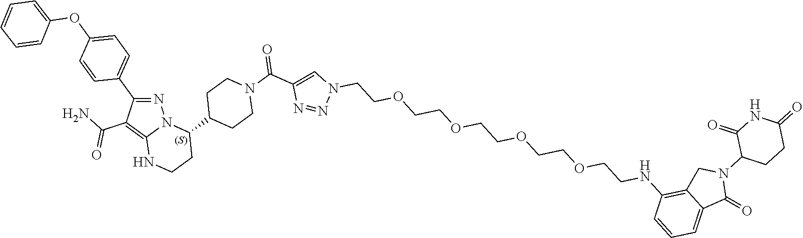 | 0.068 |
| 14 | 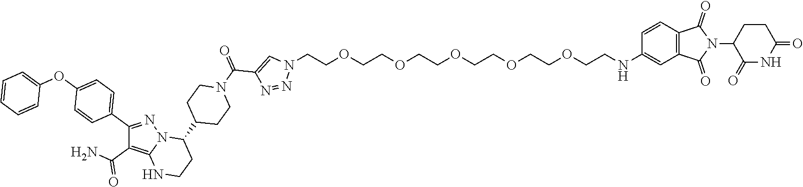 [SMILES: NC(=O)c1c(-c2ccc(Oc3ccccc3)cc2)nn2c1NCC[C@H]2C1CCN(C(=O)c2cn(CCOCCOCCOCCOCCOCCNc3ccc4c(c3)C(=O)N(C3CCC(=O)NC3=O)C4=O)nn2)CC1] | 0.078 |
| 15 | 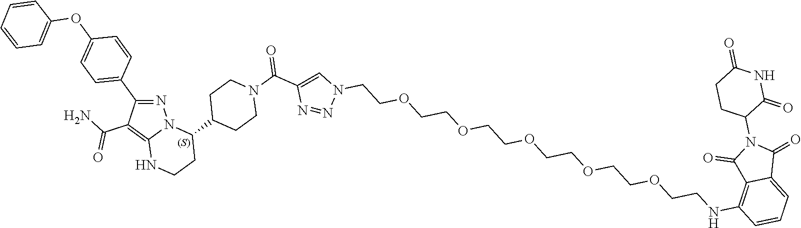 | 0.078 |
| 16 | 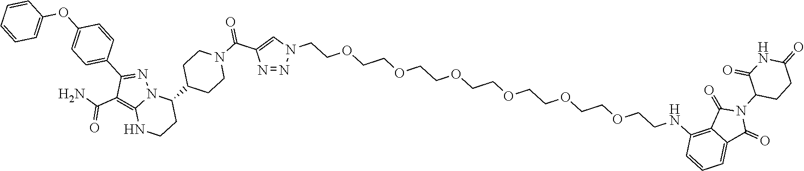 [SMILES: NC(=O)c1c(-c2ccc(Oc3ccccc3)cc2)nn2c1NCC[C@H]2C1CCN(C(=O)c2cn(CCOCCOCCOCCOCCOCCOCCNc3cccc4c3C(=O)N(C3CCC(=O)NC3=O)C4=O)nn2)CC1] | 0.107 |
| 17 | 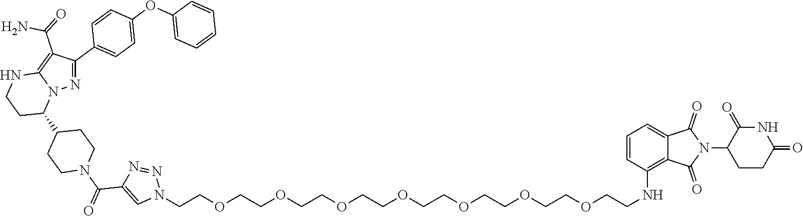 [SMILES: NC(=O)c1c(-c2ccc(Oc3ccccc3)cc2)nn2c1NCC[C@H]2C1CCN(C(=O)c2cn(CCOCCOCCOCCOCCOCCOCCOCCNc3cccc4c3C(=O)N(C3CCC(=O)NC3=O)C4=O)nn2)CC1] | >10 |
| 18 | 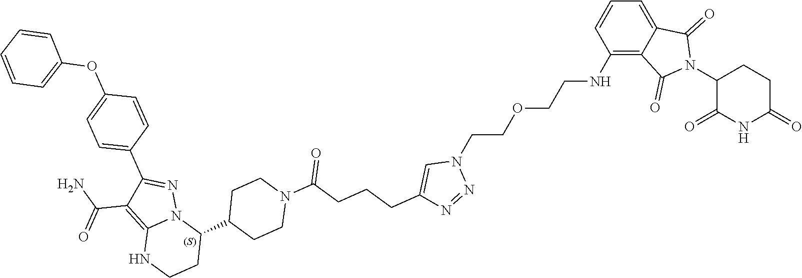 | 0.236 |
| 19 | 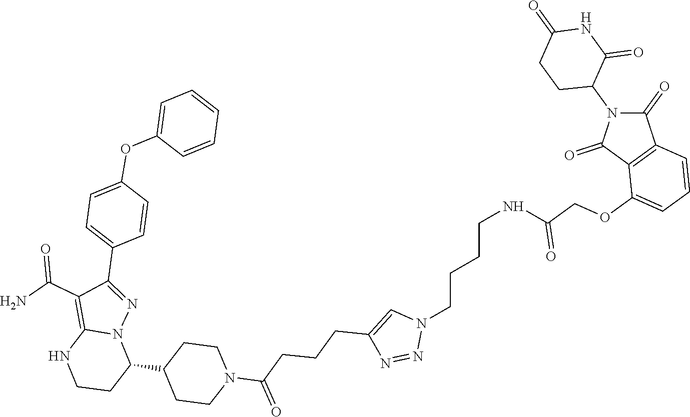 [SMILES: NC(=O)c1c(-c2ccc(Oc3ccccc3)cc2)nn2c1NCC[C@H]2C1CCN(C(=O)CCCc2cn(CCCCNC(=O)COc3cccc4c3C(=O)N(C3CCC(=O)NC3=O)C4=O)nn2)CC1] | 6.41 |
| 20 | 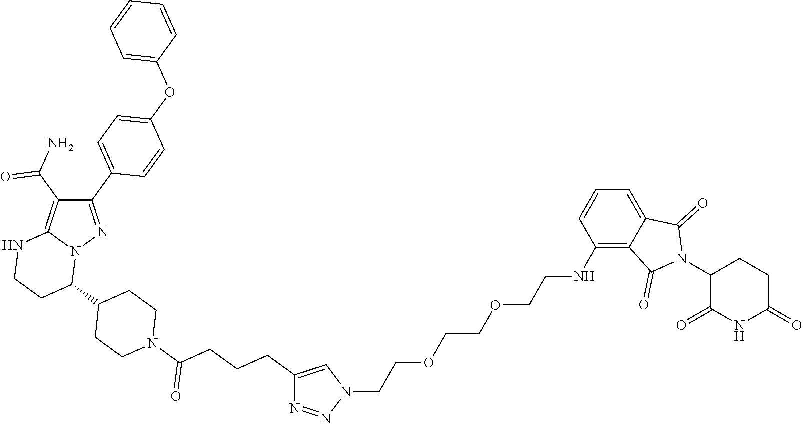 [SMILES: NC(=O)c1c(-c2ccc(Oc3ccccc3)cc2)nn2c1NCC[C@H]2C1CCN(C(=O)CCCc2cn(CCOCCOCCNc3cccc4c3C(=O)N(C3CCC(=O)NC3=O)C4=O)nn2)CC1] | >10 |
| 21 | 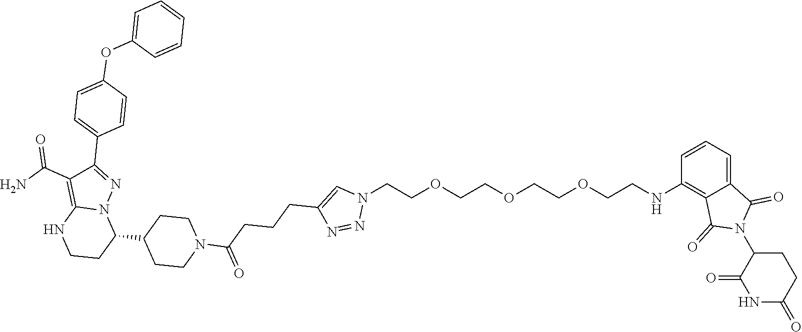 [SMILES: NC(=O)c1c(-c2ccc(Oc3ccccc3)cc2)nn2c1NCC[C@H]2C1CCN(C(=O)CCCc2cn(CCOCCOCCOCCNc3cccc4c3C(=O)N(C3CCC(=O)NC3=O)C4=O)nn2)CC1] | 0.388 |
| 22 | 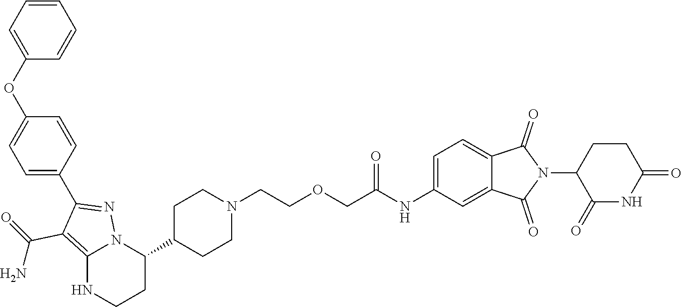 [SMILES: NC(=O)c1c(-c2ccc(Oc3ccccc3)cc2)nn2c1NCC[C@H]2C1CCN(CCOCC(=O)Nc2ccc3c(c2)C(=O)N(C2CCC(=O)NC2=O)C3=O)CC1] | |
| 23 | 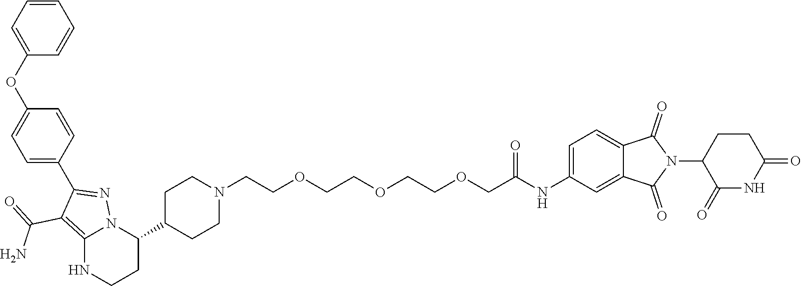 [SMILES: NC(=O)c1c(-c2ccc(Oc3ccccc3)cc2)nn2c1NCC[C@H]2C1CCN(CCOCCOCCOCC(=O)Nc2ccc3c(c2)C(=O)N(C2CCC(=O)NC2=O)C3=O)CC1] | |
| 24 | 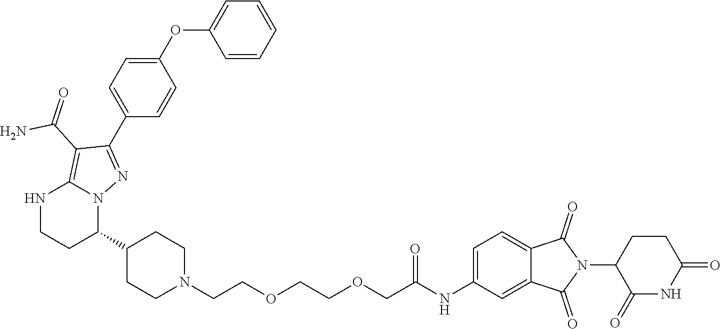 [SMILES: NC(=O)c1c(-c2ccc(Oc3ccccc3)cc2)nn2c1NCC[C@H]2C1CCN(CCOCCOCC(=O)Nc2ccc3c(c2)C(=O)N(C2CCC(=O)NC2=O)C3=O)CC1] | 0.034 |
| 25 | 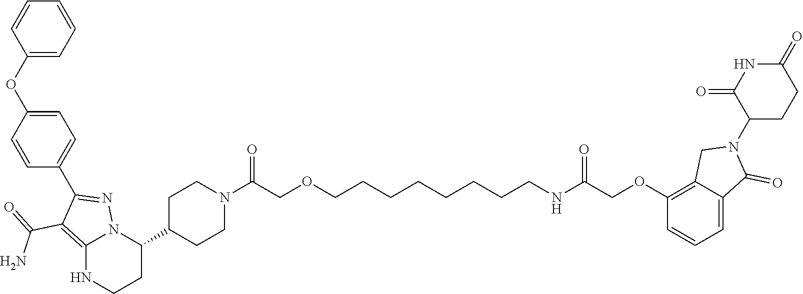 [SMILES: NC(=O)c1c(-c2ccc(Oc3ccccc3)cc2)nn2c1NCC[C@H]2C1CCN(C(=O)COCCCCCCCCNC(=O)COc2cccc3c2CN(C2CCC(=O)NC2=O)C3=O)CC1] | >10 |
| 26 | 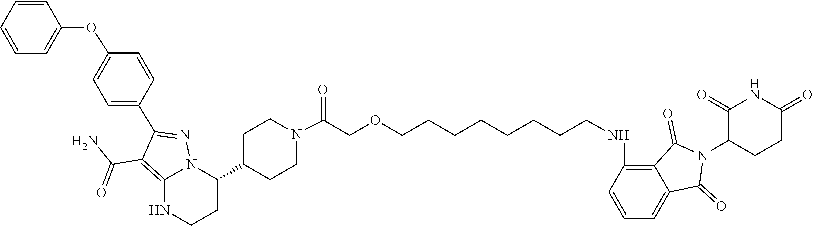 [SMILES: NC(=O)c1c(-c2ccc(Oc3ccccc3)cc2)nn2c1NCC[C@H]2C1CCN(C(=O)COCCCCCCCCNc2cccc3c2C(=O)N(C2CCC(=O)NC2=O)C3=O)CC1] | >10 |
| 27 | 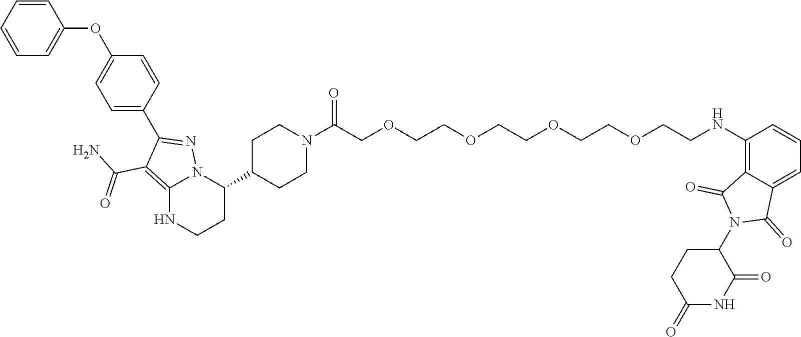 [SMILES: NC(=O)c1c(-c2ccc(Oc3ccccc3)cc2)nn2c1NCC[C@H]2C1CCN(C(=O)COCCOCCOCCOCCNc2cccc3c2C(=O)N(C2CCC(=O)NC2=O)C3=O)CC1] | >10 |
| 28 | 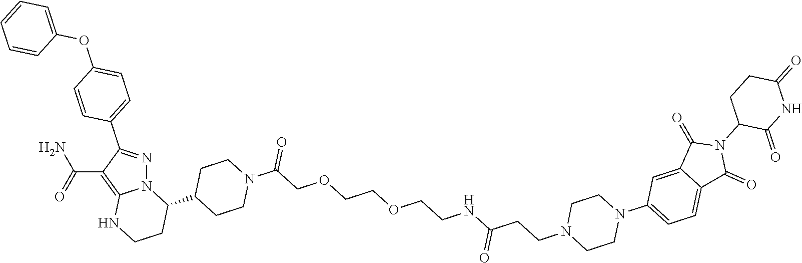 [SMILES: NC(=O)c1c(-c2ccc(Oc3ccccc3)cc2)nn2c1NCC[C@H]2C1CCN(C(=O)COCCOCCNC(=O)CCN2CCN(c3ccc4c(c3)C(=O)N(C3CCC(=O)NC3=O)C4=O)CC2)CC1] | 1.48 |
| 29 | 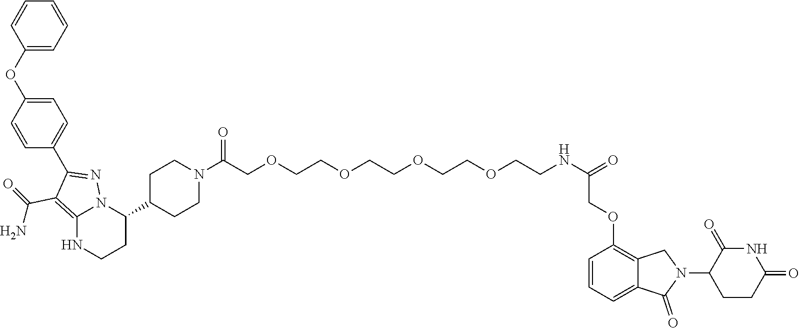 [SMILES: NC(=O)c1c(-c2ccc(Oc3ccccc3)cc2)nn2c1NCC[C@H]2C1CCN(C(=O)COCCOCCOCCOCCNC(=O)COc2cccc3c2CN(C2CCC(=O)NC2=O)C3=O)CC1] | 0.458 |
| 30 | 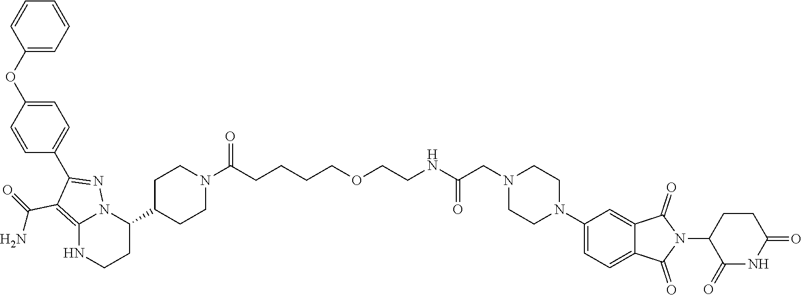 [SMILES: NC(=O)c1c(-c2ccc(Oc3ccccc3)cc2)nn2c1NCC[C@H]2C1CCN(C(=O)CCCCOCCNC(=O)CN2CCN(c3ccc4c(c3)C(=O)N(C3CCC(=O)NC3=O)C4=O)CC2)CC1] | |
| 31 | 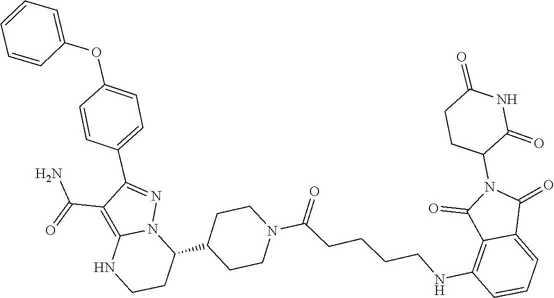 [SMILES: NC(=O)c1c(-c2ccc(Oc3ccccc3)cc2)nn2c1NCC[C@H]2C1CCN(C(=O)CCCCNc2cccc3c2C(=O)N(C2CCC(=O)NC2=O)C3=O)CC1] | >10 |
| 32 | 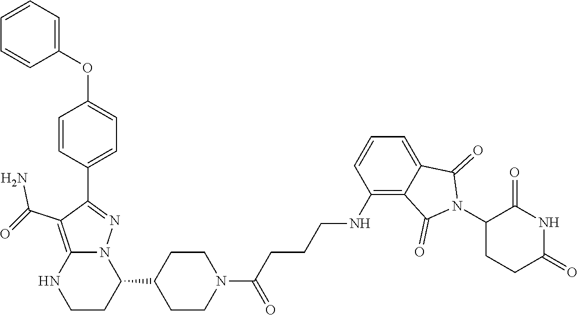 [SMILES: NC(=O)c1c(-c2ccc(Oc3ccccc3)cc2)nn2c1NCC[C@H]2C1CCN(C(=O)CCCNc2cccc3c2C(=O)N(C2CCC(=O)NC2=O)C3=O)CC1] | >10 |
| 33 | 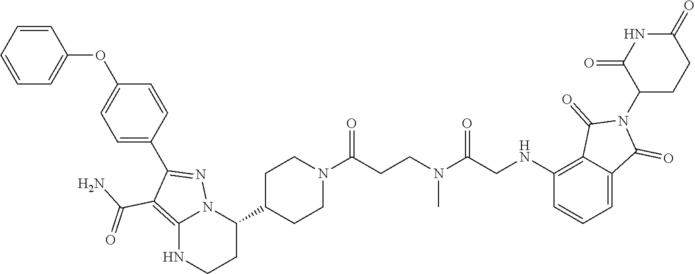 [SMILES: CN(CCC(=O)N1CCC([C@@H]2CCNc3c(C(N)=O)c(-c4ccc(Oc5ccccc5)cc4)nn32)CC1)C(=O)CNc1cccc2c1C(=O)N(C1CCC(=O)NC1=O)C2=O] | |
| 34 | 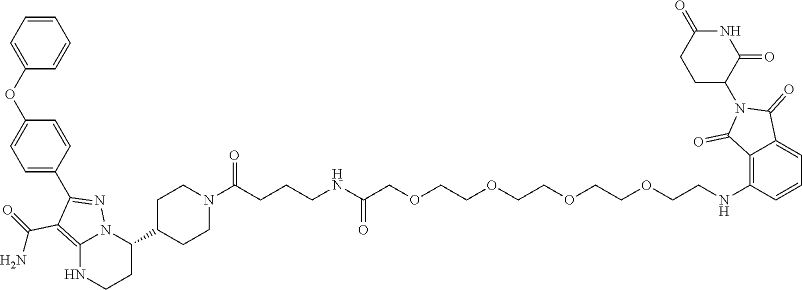 [SMILES: NC(=O)c1c(-c2ccc(Oc3ccccc3)cc2)nn2c1NCC[C@H]2C1CCN(C(=O)CCCNC(=O)COCCOCCOCCOCCNc2cccc3c2C(=O)N(C2CCC(=O)NC2=O)C3=O)CC1] | 1.89 |
| 35 | 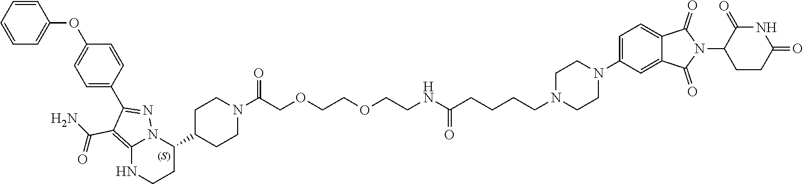 | 0.326 |
| 36 | 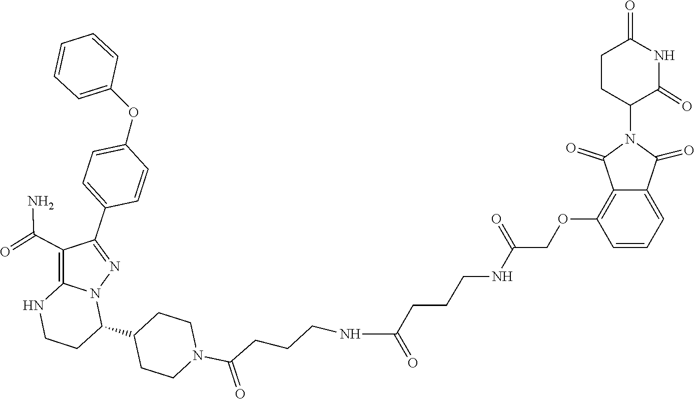 [SMILES: NC(=O)c1c(-c2ccc(Oc3ccccc3)cc2)nn2c1NCC[C@H]2C1CCN(C(=O)CCCNC(=O)CCCNC(=O)COc2cccc3c2C(=O)N(C2CCC(=O)NC2=O)C3=O)CC1] | |
| 37 | 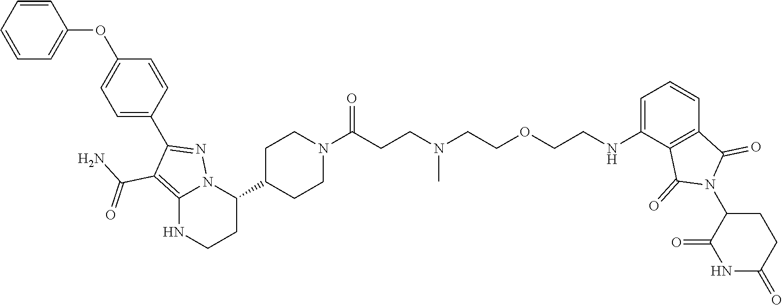 [SMILES: CN(CCOCCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1] | 0.12 |
| 38 | 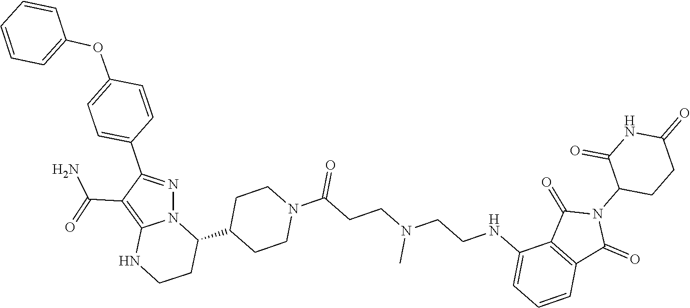 [SMILES: CN(CCNc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1] | 0.371 |
| 39 | 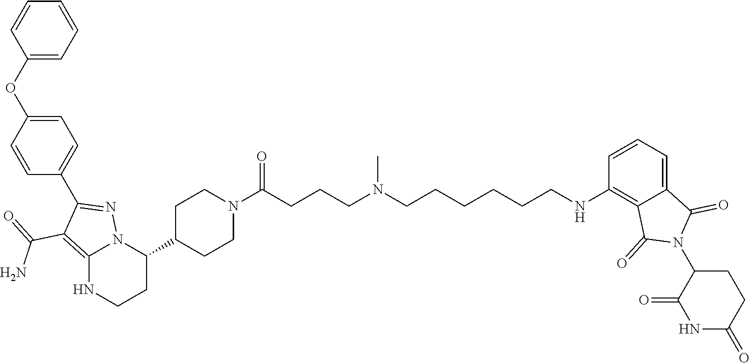 [SMILES: CN(CCCCCCNc1cccc2c1C(=O)N(C1CCC(=O)NC1=O)C2=O)CCCC(=O)N1CCC([C@@H]2CCNc3c(C(N)=O)c(-c4ccc(Oc5ccccc5)cc4)nn32)CC1] | |
| 40 | 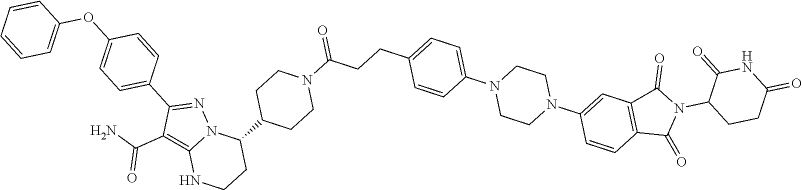 [SMILES: NC(=O)c1c(-c2ccc(Oc3ccccc3)cc2)nn2c1NCC[C@H]2C1CCN(C(=O)CCc2ccc(N3CCN(c4ccc5c(c4)C(=O)N(C4CCC(=O)NC4=O)C5=O)CC3)cc2)CC1] | 0.074 |
| 41 | 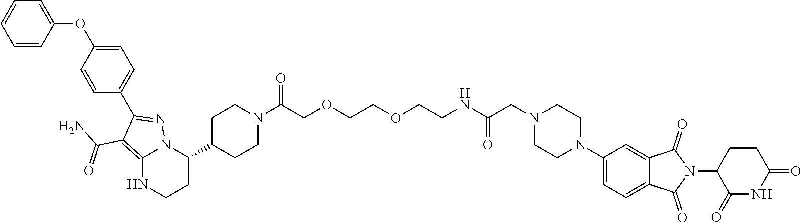 [SMILES: NC(=O)c1c(-c2ccc(Oc3ccccc3)cc2)nn2c1NCC[C@H]2C1CCN(C(=O)COCCOCCNC(=O)CN2CCN(c3ccc4c(c3)C(=O)N(C3CCC(=O)NC3=O)C4=O)CC2)CC1] | |
| 42 | 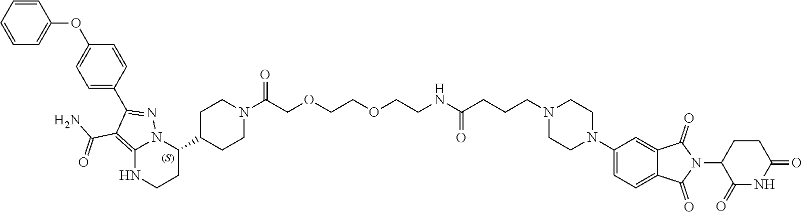 | 0.624 |
| 43 | 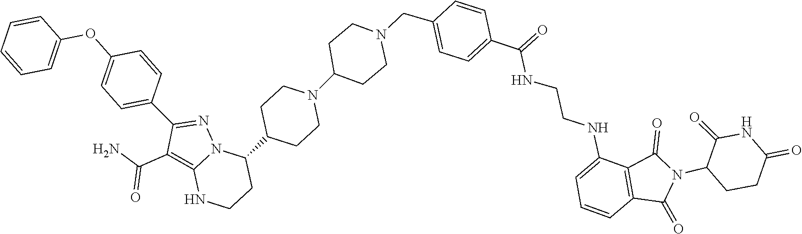 [SMILES: NC(=O)c1c(-c2ccc(Oc3ccccc3)cc2)nn2c1NCC[C@H]2C1CCN(C2CCN(Cc3ccc(C(=O)NCCNc4cccc5c4C(=O)N(C4CCC(=O)NC4=O)C5=O)cc3)CC2)CC1] | >10 |
| 44 | 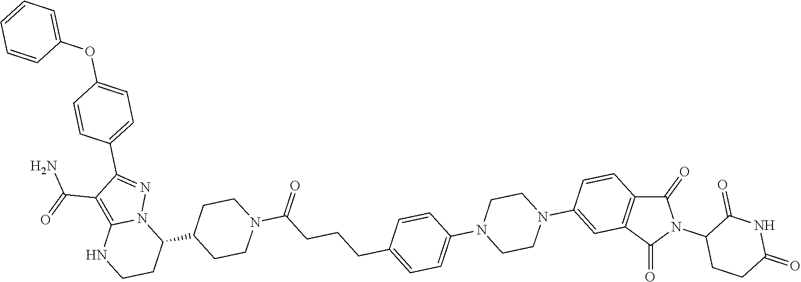 [SMILES: NC(=O)c1c(-c2ccc(Oc3ccccc3)cc2)nn2c1NCC[C@H]2C1CCN(C(=O)CCCc2ccc(N3CCN(c4ccc5c(c4)C(=O)N(C4CCC(=O)NC4=O)C5=O)CC3)cc2)CC1] | >10 |
| 45 | 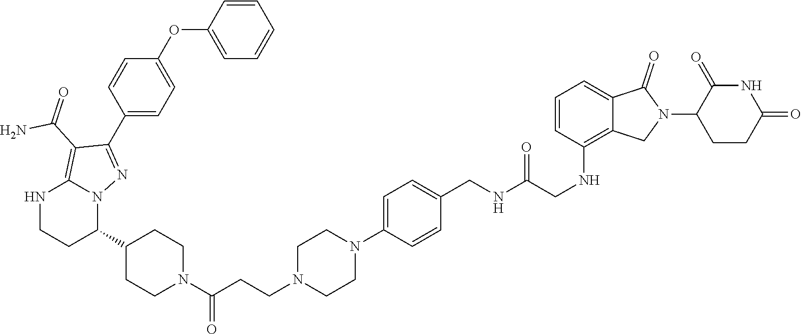 [SMILES: NC(=O)c1c(-c2ccc(Oc3ccccc3)cc2)nn2c1NCC[C@H]2C1CCN(C(=O)CCN2CCN(c3ccc(CNC(=O)CNc4cccc5c4CN(C4CCC(=O)NC4=O)C5=O)cc3)CC2)CC1] | 0.382 |
| 46 | 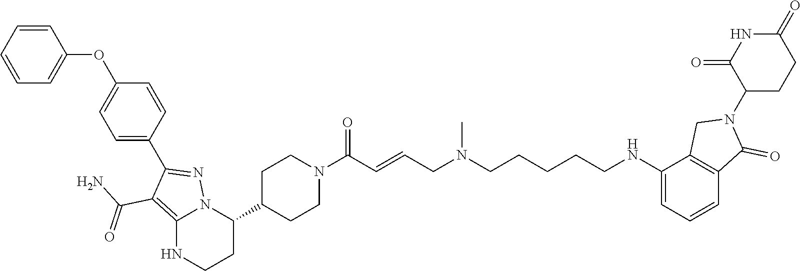 [SMILES: CN(C/C=C/C(=O)N1CCC([C@@H]2CCNc3c(C(N)=O)c(-c4ccc(Oc5ccccc5)cc4)nn32)CC1)CCCCCNc1cccc2c1CN(C1CCC(=O)NC1=O)C2=O] | 0.198 |
| 47 | 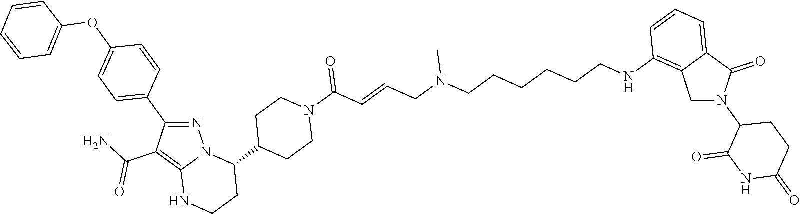 [SMILES: CN(C/C=C/C(=O)N1CCC([C@@H]2CCNc3c(C(N)=O)c(-c4ccc(Oc5ccccc5)cc4)nn32)CC1)CCCCCCNc1cccc2c1CN(C1CCC(=O)NC1=O)C2=O] | 0.315 |
| 48 | 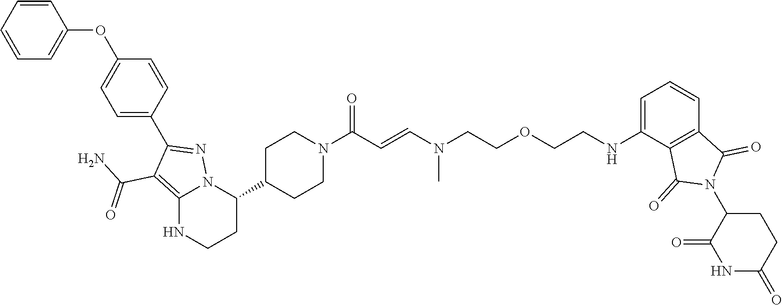 [SMILES: CN(/C=C/C(=O)N1CCC([C@@H]2CCNc3c(C(N)=O)c(-c4ccc(Oc5ccccc5)cc4)nn32)CC1)CCOCCNc1cccc2c1C(=O)N(C1CCC(=O)NC1=O)C2=O] | |
| 49 | 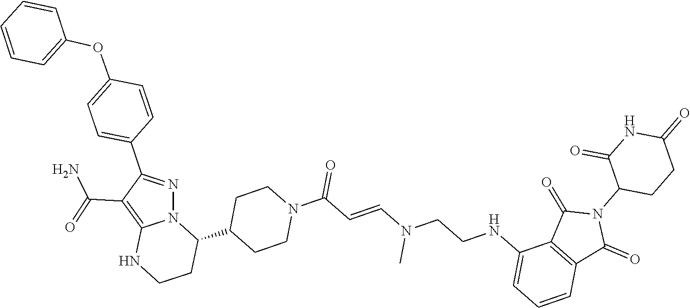 [SMILES: CN(/C=C/C(=O)N1CCC([C@@H]2CCNc3c(C(N)=O)c(-c4ccc(Oc5ccccc5)cc4)nn32)CC1)CCNc1cccc2c1C(=O)N(C1CCC(=O)NC1=O)C2=O] | >10 |
| 50 | 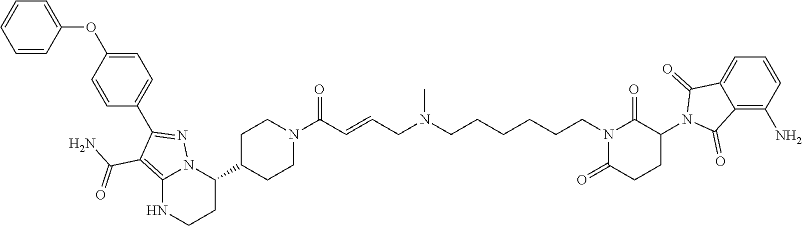 [SMILES: CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2C(=O)c3cccc(N)c3C2=O)C1=O] | >10 |
| 51 | 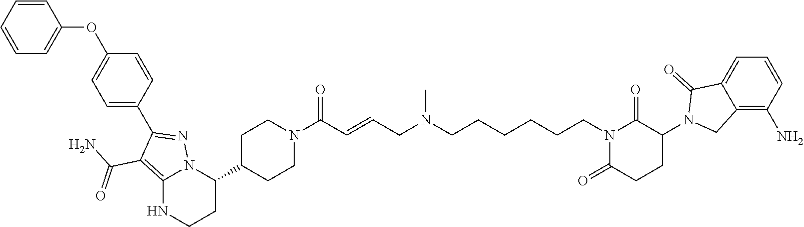 [SMILES: CN(C/C=C/C(=O)N1CCC([C@@H]2CCNc3c(C(N)=O)c(-c4ccc(Oc5ccccc5)cc4)nn32)CC1)CCCCCCN1C(=O)CCC(N2Cc3c(N)cccc3C2=O)C1=O] | >10 |
| 52 | 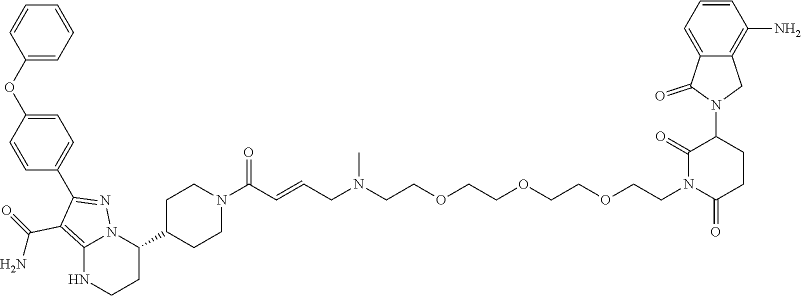 [SMILES: CN(C/C=C/C(=O)N1CCC([C@@H]2CCNc3c(C(N)=O)c(-c4ccc(Oc5ccccc5)cc4)nn32)CC1)CCOCCOCCOCCN1C(=O)CCC(N2Cc3c(N)cccc3C2=O)C1=O] | 0.144 |
| 53 | 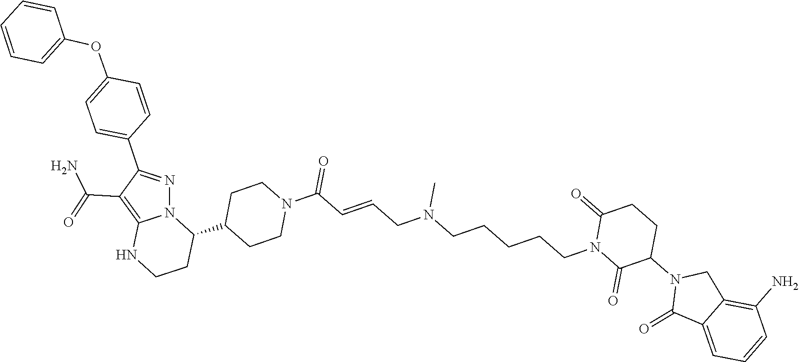 [SMILES: CN(C/C=C/C(=O)N1CCC([C@@H]2CCNc3c(C(N)=O)c(-c4ccc(Oc5ccccc5)cc4)nn32)CC1)CCCCCN1C(=O)CCC(N2Cc3c(N)cccc3C2=O)C1=O] | 37.2 |
| 54 | 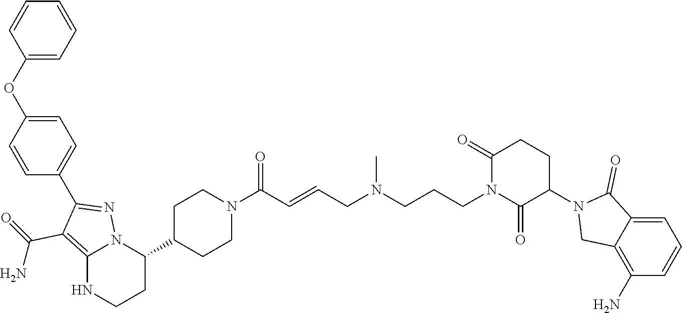 [SMILES: CN(C/C=C/C(=O)N1CCC([C@@H]2CCNc3c(C(N)=O)c(-c4ccc(Oc5ccccc5)cc4)nn32)CC1)CCCN1C(=O)CCC(N2Cc3c(N)cccc3C2=O)C1=O] | 0.428 |
| 55 | 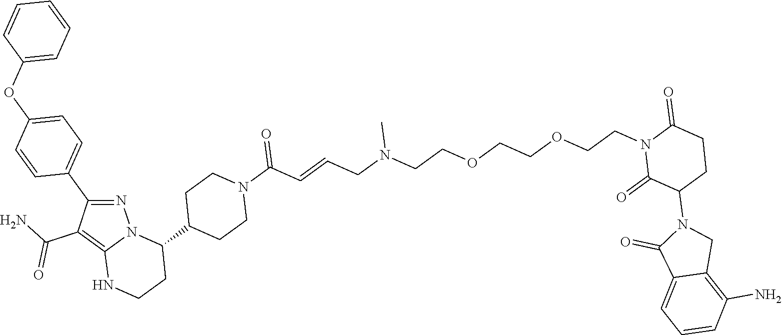 [SMILES: CN(C/C=C/C(=O)N1CCC([C@@H]2CCNc3c(C(N)=O)c(-c4ccc(Oc5ccccc5)cc4)nn32)CC1)CCOCCOCCN1C(=O)CCC(N2Cc3c(N)cccc3C2=O)C1=O] | 0.382 |
| 56 | 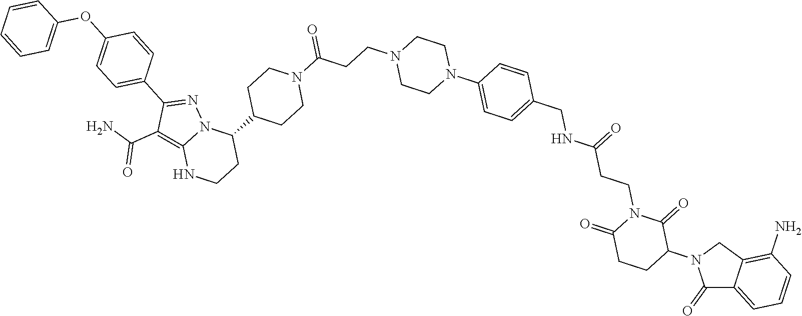 [SMILES: NC(=O)c1c(-c2ccc(Oc3ccccc3)cc2)nn2c1NCC[C@H]2C1CCN(C(=O)CCN2CCN(c3ccc(CNC(=O)CCN4C(=O)CCC(N5Cc6c(N)cccc6C5=O)C4=O)cc3)CC2)CC1] | 2.36 |
| 57 | 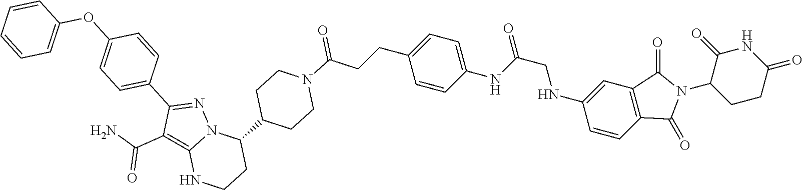 [SMILES: NC(=O)c1c(-c2ccc(Oc3ccccc3)cc2)nn2c1NCC[C@H]2C1CCN(C(=O)CCc2ccc(NC(=O)CNc3ccc4c(c3)C(=O)N(C3CCC(=O)NC3=O)C4=O)cc2)CC1] | >10 |
| 58 | 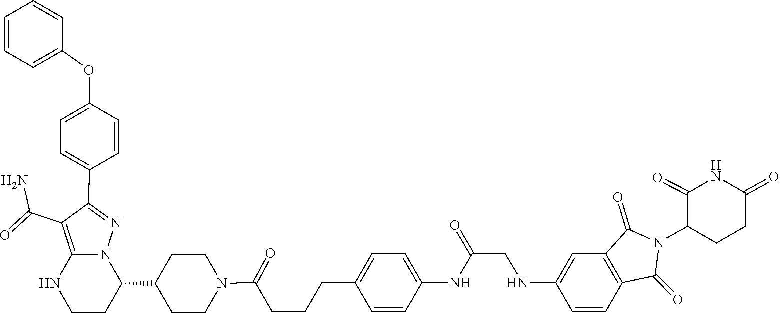 [SMILES: NC(=O)c1c(-c2ccc(Oc3ccccc3)cc2)nn2c1NCC[C@H]2C1CCN(C(=O)CCCc2ccc(NC(=O)CNc3ccc4c(c3)C(=O)N(C3CCC(=O)NC3=O)C4=O)cc2)CC1] | 0.028 |
| 59 | 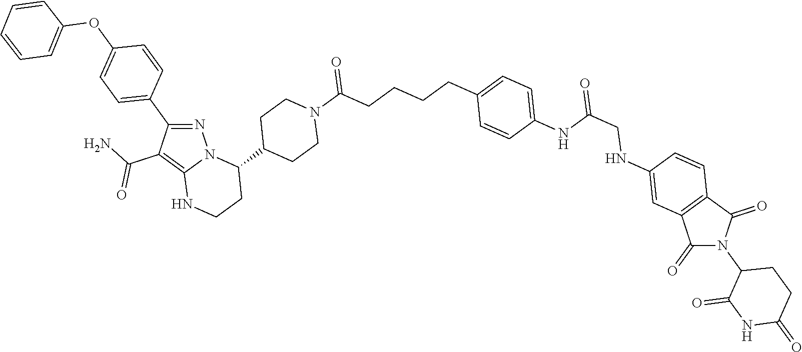 [SMILES: NC(=O)c1c(-c2ccc(Oc3ccccc3)cc2)nn2c1NCC[C@H]2C1CCN(C(=O)CCCCc2ccc(NC(=O)CNc3ccc4c(c3)C(=O)N(C3CCC(=O)NC3=O)C4=O)cc2)CC1] | |
| 60 | 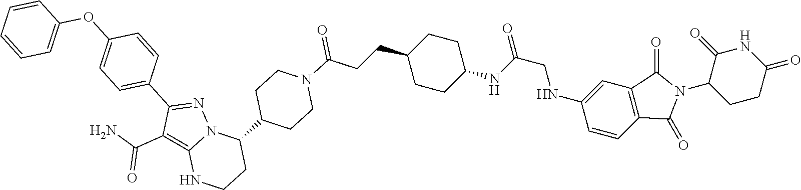 [SMILES: NC(=O)c1c(-c2ccc(Oc3ccccc3)cc2)nn2c1NCC[C@H]2C1CCN(C(=O)CC[C@H]2CC[C@H](NC(=O)CNc3ccc4c(c3)C(=O)N(C3CCC(=O)NC3=O)C4=O)CC2)CC1] | |
| 61 | 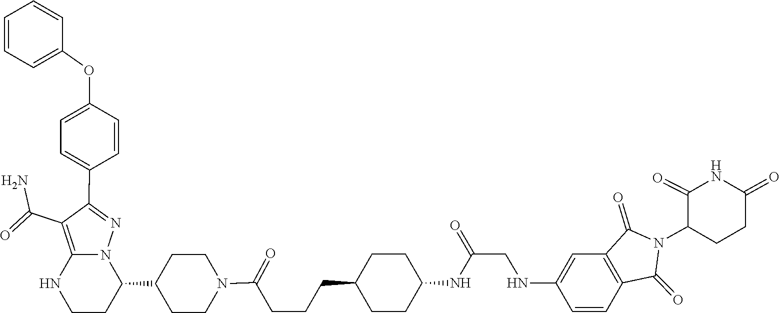 [SMILES: NC(=O)c1c(-c2ccc(Oc3ccccc3)cc2)nn2c1NCC[C@H]2C1CCN(C(=O)CCC[C@H]2CC[C@H](NC(=O)CNc3ccc4c(c3)C(=O)N(C3CCC(=O)NC3=O)C4=O)CC2)CC1] | |
| 62 | 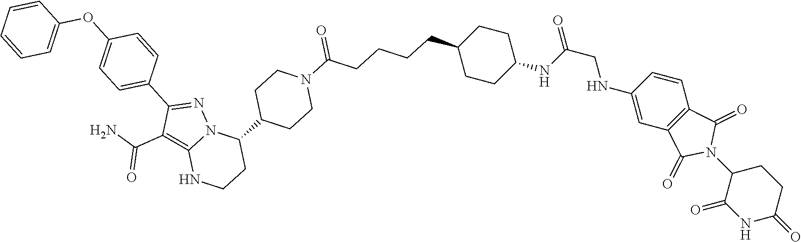 [SMILES: NC(=O)c1c(-c2ccc(Oc3ccccc3)cc2)nn2c1NCC[C@H]2C1CCN(C(=O)CCCC[C@H]2CC[C@H](NC(=O)CNc3ccc4c(c3)C(=O)N(C3CCC(=O)NC3=O)C4=O)CC2)CC1] | |
| 63 | 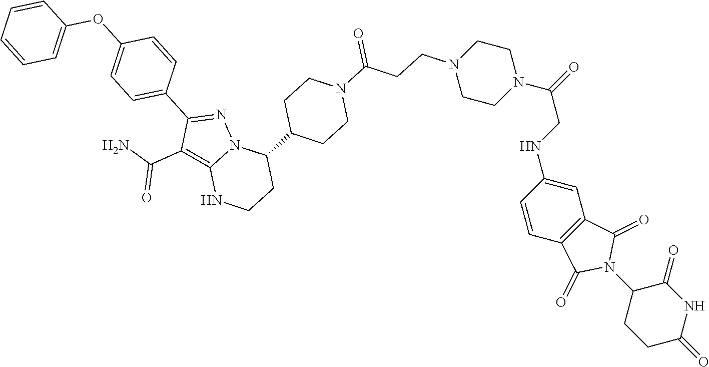 [SMILES: NC(=O)c1c(-c2ccc(Oc3ccccc3)cc2)nn2c1NCC[C@H]2C1CCN(C(=O)CCN2CCN(C(=O)CNc3ccc4c(c3)C(=O)N(C3CCC(=O)NC3=O)C4=O)CC2)CC1] | 0.0029 |
| 64 | 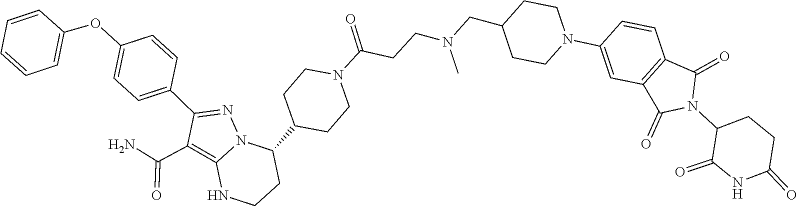 [SMILES: CN(CCC(=O)N1CCC([C@@H]2CCNc3c(C(N)=O)c(-c4ccc(Oc5ccccc5)cc4)nn32)CC1)CC1CCN(c2ccc3c(c2)C(=O)N(C2CCC(=O)NC2=O)C3=O)CC1] | 0.035 |
| 65 | 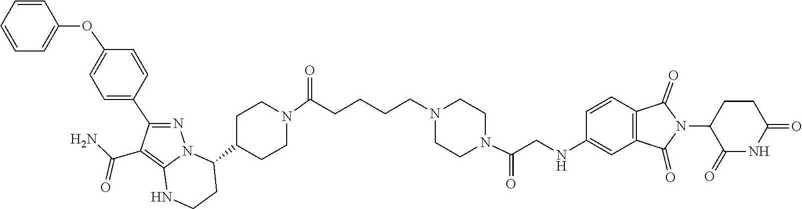 [SMILES: NC(=O)c1c(-c2ccc(Oc3ccccc3)cc2)nn2c1NCC[C@H]2C1CCN(C(=O)CCCCN2CCN(C(=O)CNc3ccc4c(c3)C(=O)N(C3CCC(=O)NC3=O)C4=O)CC2)CC1] | |
| 66 | 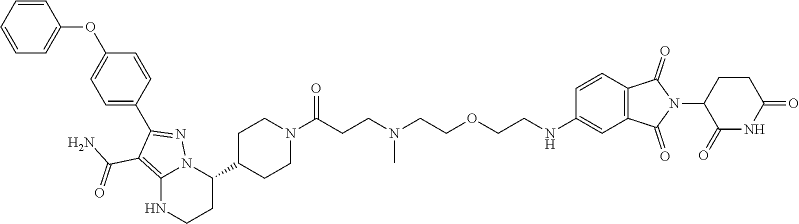 [SMILES: CN(CCOCCNc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1] | |
| 67 | 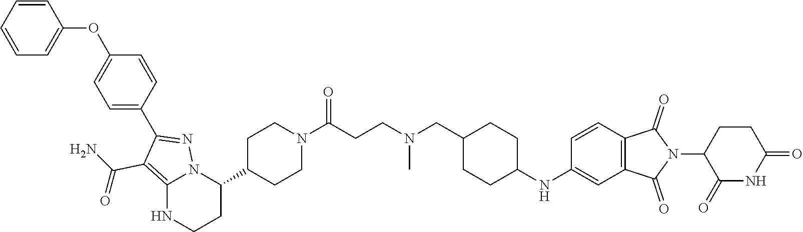 [SMILES: CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1] | |
| 68 | 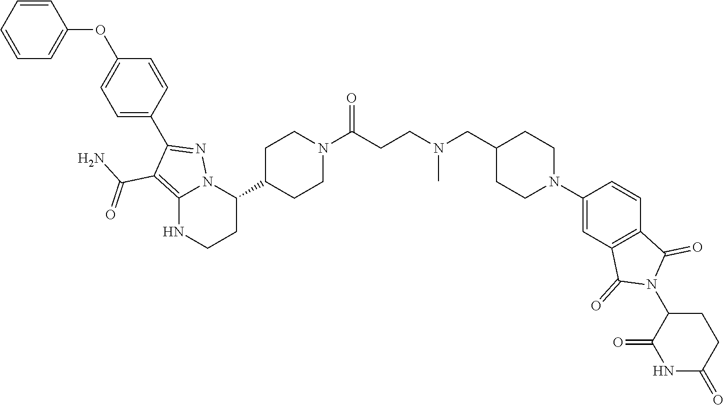 [SMILES: CN(CCC(=O)N1CCC([C@@H]2CCNc3c(C(N)=O)c(-c4ccc(Oc5ccccc5)cc4)nn32)CC1)CC1CCN(c2ccc3c(c2)C(=O)N(C2CCC(=O)NC2=O)C3=O)CC1] | |
| 69 | 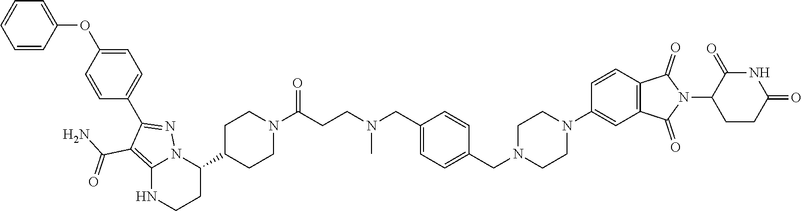 [SMILES: CN(CCC(=O)N1CCC([C@@H]2CCNc3c(C(N)=O)c(-c4ccc(Oc5ccccc5)cc4)nn32)CC1)Cc1ccc(CN2CCN(c3ccc4c(c3)C(=O)N(C3CCC(=O)NC3=O)C4=O)CC2)cc1] | |
| 70 | 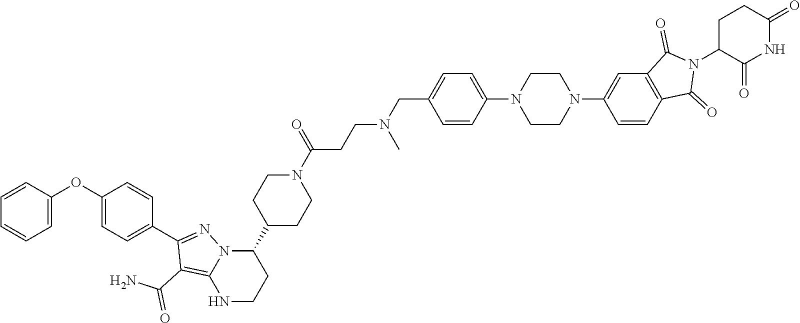 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)N(C3CCC(=O)NC3=O)C4=O)CC2)cc1] | |
| 71 | 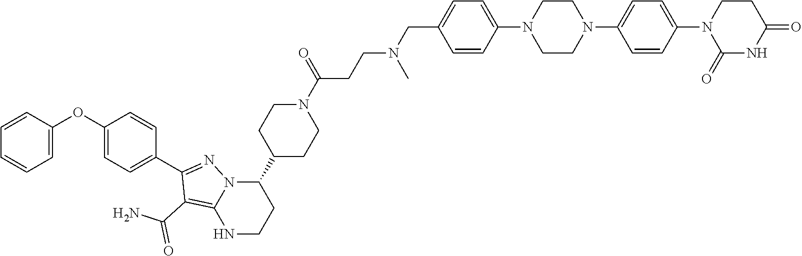 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc(N4CCC(=O)NC4=O)cc3)CC2)cc1] | |
| 72 | 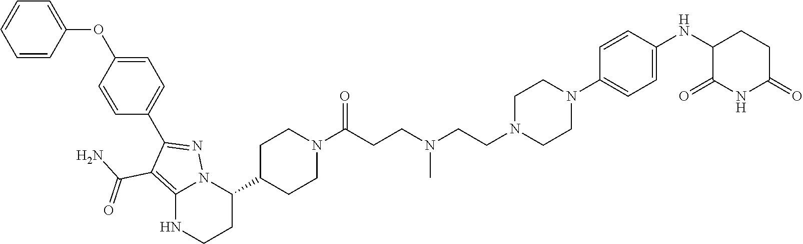 [SMILES: CN(CCC(=O)N1CCC([C@@H]2CCNc3c(C(N)=O)c(-c4ccc(Oc5ccccc5)cc4)nn32)CC1)CCN1CCN(c2ccc(NC3CCC(=O)NC3=O)cc2)CC1] | |
| 73 | 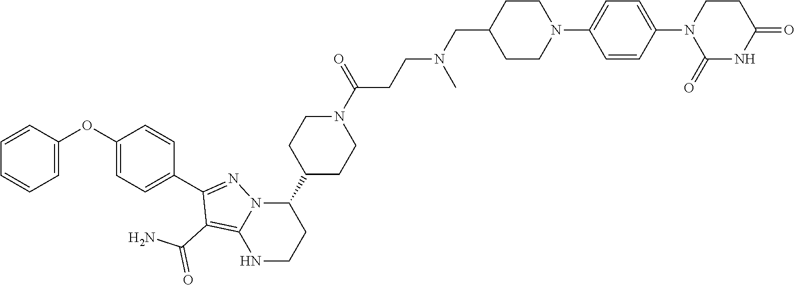 [SMILES: CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1] | |
| 74 | 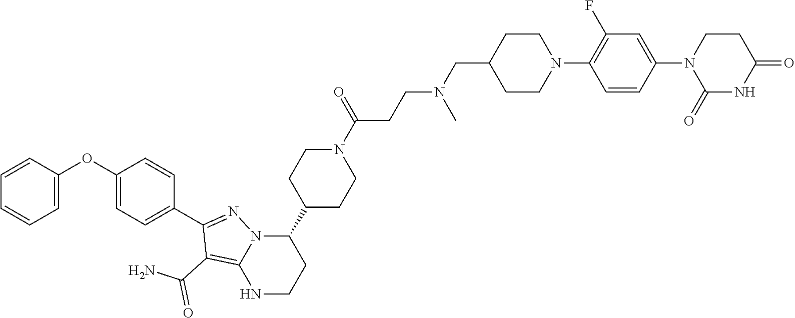 [SMILES: CN(CCC(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2F)CC1] | |
| 75 | 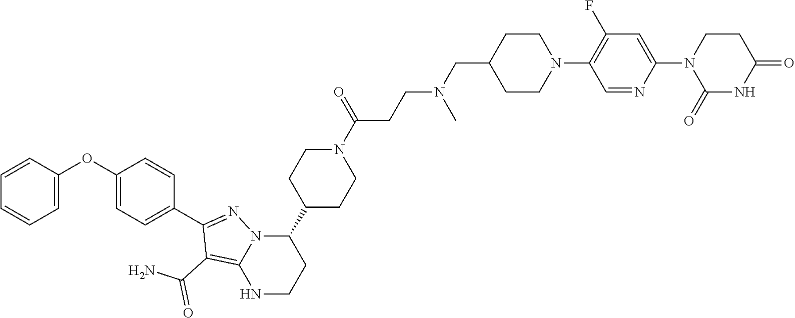 [SMILES: CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cc2F)CC1] | |
| 76 | 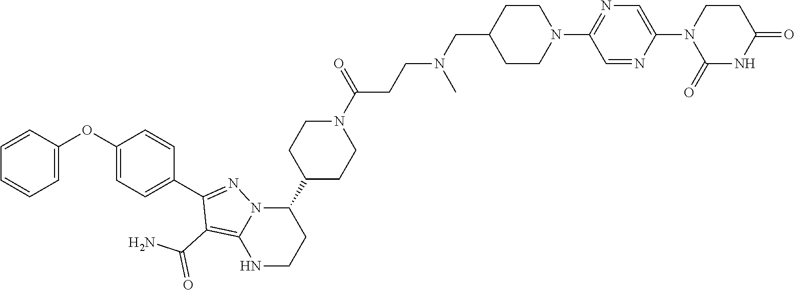 [SMILES: CN(CCC(=O)N1CCC([C@@H]2CCNc3c(C(N)=O)c(-c4ccc(Oc5ccccc5)cc4)nn32)CC1)CC1CCN(c2cnc(N3CCC(=O)NC3=O)cn2)CC1] | |
| 77 | 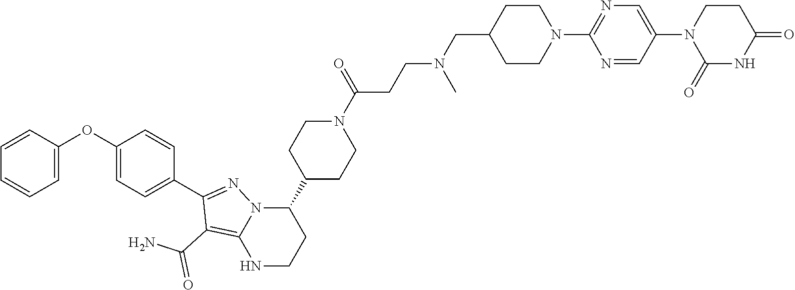 [SMILES: CN(CCC(=O)N1CCC([C@@H]2CCNc3c(C(N)=O)c(-c4ccc(Oc5ccccc5)cc4)nn32)CC1)CC1CCN(c2ncc(N3CCC(=O)NC3=O)cn2)CC1] | |
| 78 | 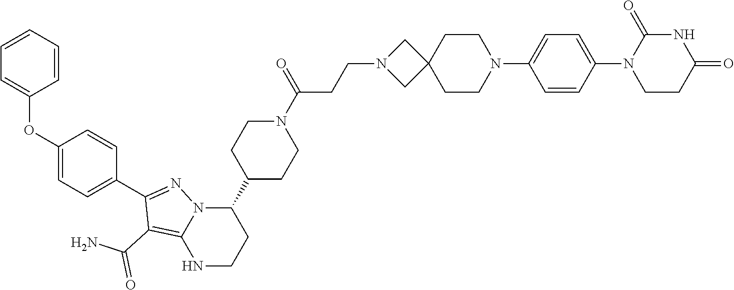 [SMILES: NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1] | |
| 79 | 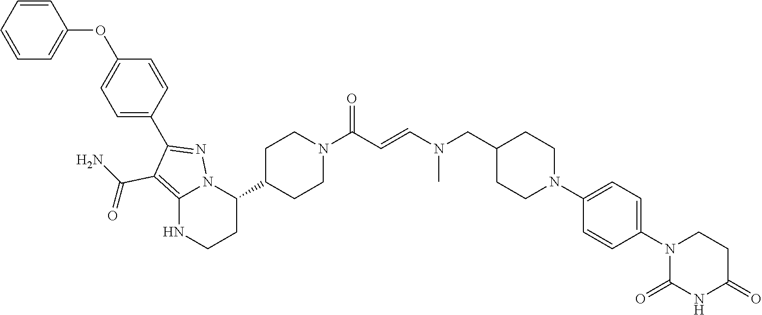 [SMILES: CN(/C=C/C(=O)N1CCC([C@@H]2CCNc3c(C(N)=O)c(-c4ccc(Oc5ccccc5)cc4)nn32)CC1)CC1CCN(c2ccc(N3CCC(=O)NC3=O)cc2)CC1] | |
| 80 | 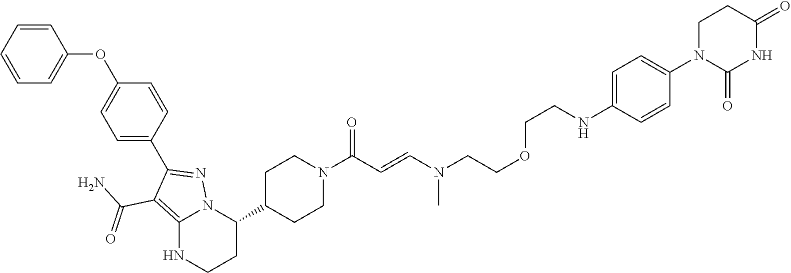 [SMILES: CN(/C=C/C(=O)N1CCC([C@@H]2CCNc3c(C(N)=O)c(-c4ccc(Oc5ccccc5)cc4)nn32)CC1)CCOCCNc1ccc(N2CCC(=O)NC2=O)cc1] | |
| 81 | 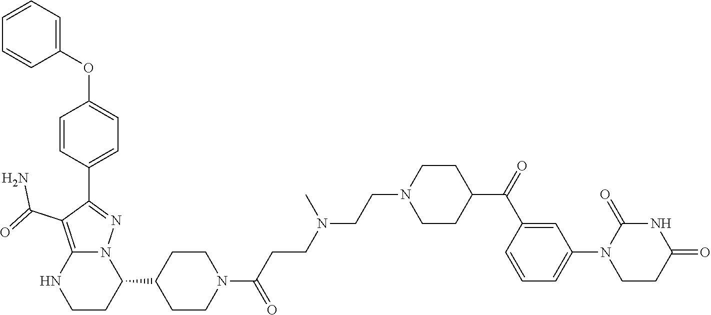 [SMILES: CN(CCC(=O)N1CCC([C@@H]2CCNc3c(C(N)=O)c(-c4ccc(Oc5ccccc5)cc4)nn32)CC1)CCN1CCC(C(=O)c2cccc(N3CCC(=O)NC3=O)c2)CC1] | |
| 82 | 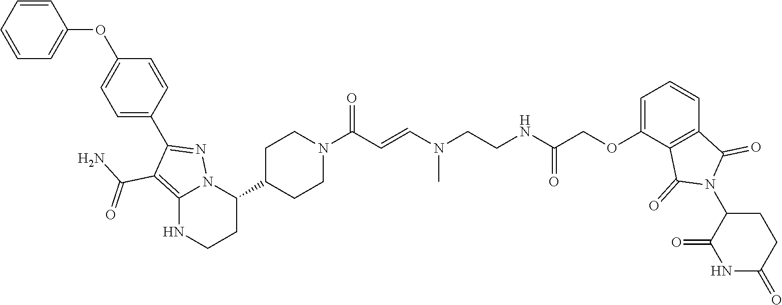 [SMILES: CN(/C=C/C(=O)N1CCC([C@@H]2CCNc3c(C(N)=O)c(-c4ccc(Oc5ccccc5)cc4)nn32)CC1)CCNC(=O)COc1cccc2c1C(=O)N(C1CCC(=O)NC1=O)C2=O] | |
| 83 | 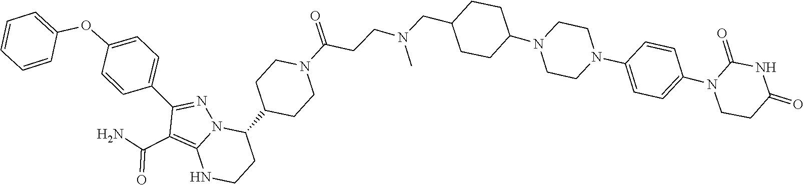 [SMILES: CN(CCC(=O)N1CCC([C@@H]2CCNc3c(C(N)=O)c(-c4ccc(Oc5ccccc5)cc4)nn32)CC1)CC1CCC(N2CCN(c3ccc(N4CCC(=O)NC4=O)cc3)CC2)CC1] | |
| 84 | 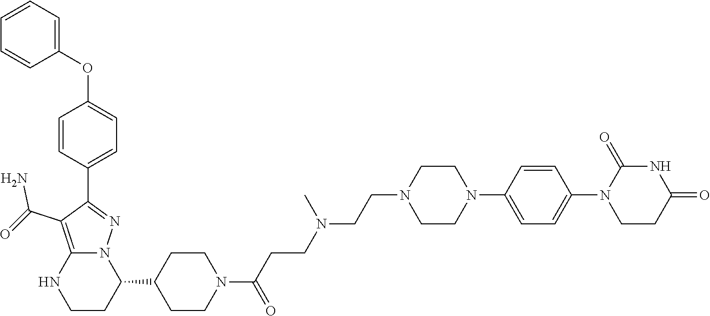 [SMILES: CN(CCC(=O)N1CCC([C@@H]2CCNc3c(C(N)=O)c(-c4ccc(Oc5ccccc5)cc4)nn32)CC1)CCN1CCN(c2ccc(N3CCC(=O)NC3=O)cc2)CC1] | |
| 85 | 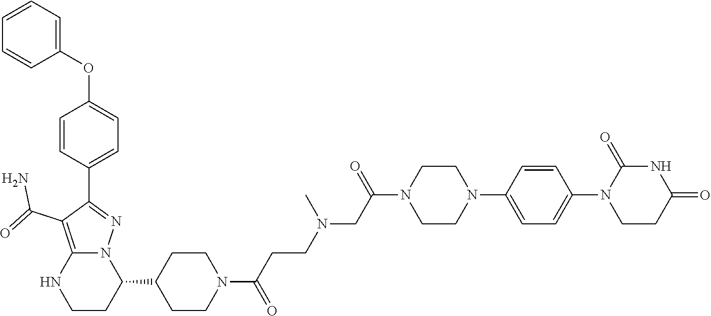 [SMILES: CN(CCC(=O)N1CCC([C@@H]2CCNc3c(C(N)=O)c(-c4ccc(Oc5ccccc5)cc4)nn32)CC1)CC(=O)N1CCN(c2ccc(N3CCC(=O)NC3=O)cc2)CC1] | |
| 86 | 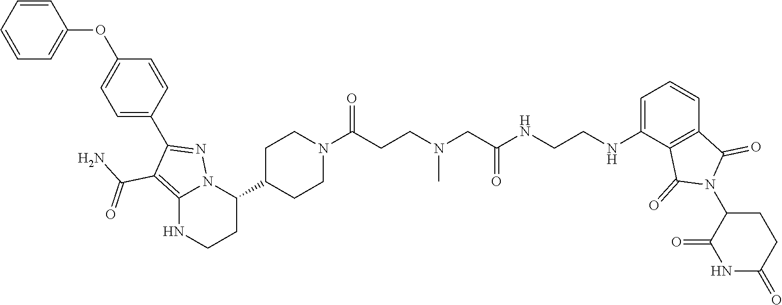 [SMILES: CN(CCC(=O)N1CCC([C@@H]2CCNc3c(C(N)=O)c(-c4ccc(Oc5ccccc5)cc4)nn32)CC1)CC(=O)NCCNc1cccc2c1C(=O)N(C1CCC(=O)NC1=O)C2=O] | |
| 87 | 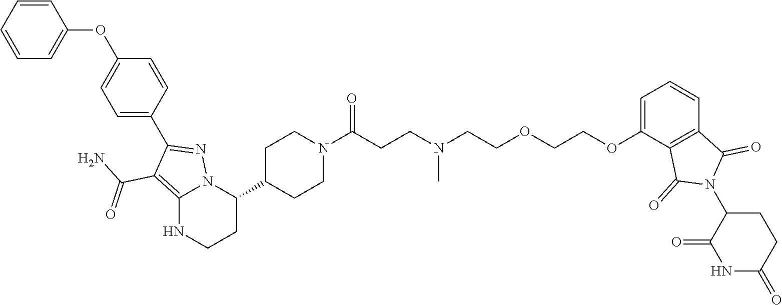 [SMILES: CN(CCOCCOc1cccc2c1C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1] | |
| 88 | 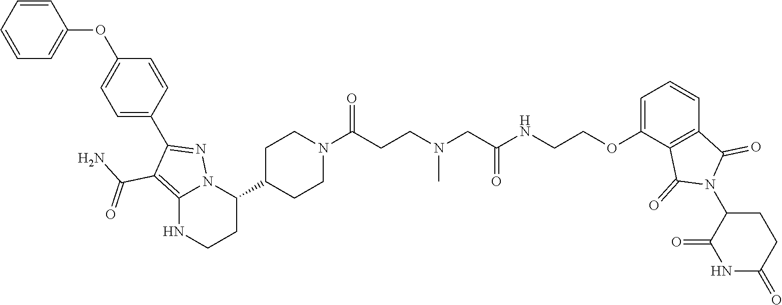 [SMILES: CN(CCC(=O)N1CCC([C@@H]2CCNc3c(C(N)=O)c(-c4ccc(Oc5ccccc5)cc4)nn32)CC1)CC(=O)NCCOc1cccc2c1C(=O)N(C1CCC(=O)NC1=O)C2=O] | |
| 89 | 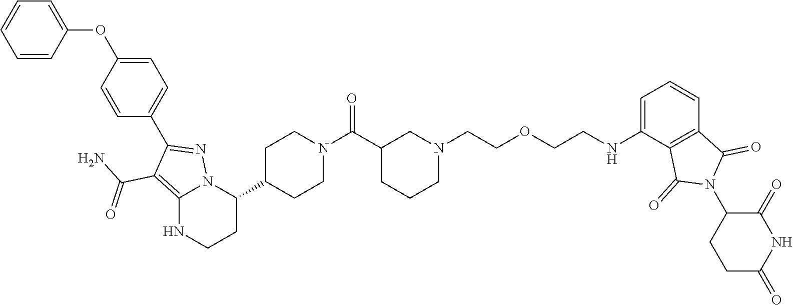 [SMILES: NC(=O)c1c(-c2ccc(Oc3ccccc3)cc2)nn2c1NCC[C@H]2C1CCN(C(=O)C2CCCN(CCOCCNc3cccc4c3C(=O)N(C3CCC(=O)NC3=O)C4=O)C2)CC1] | |
| 90 | 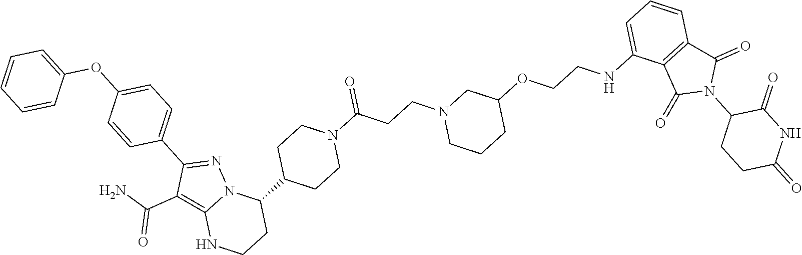 [SMILES: NC(=O)c1c(-c2ccc(Oc3ccccc3)cc2)nn2c1NCC[C@H]2C1CCN(C(=O)CCN2CCCC(OCCNc3cccc4c3C(=O)N(C3CCC(=O)NC3=O)C4=O)C2)CC1] | |
| 91 | 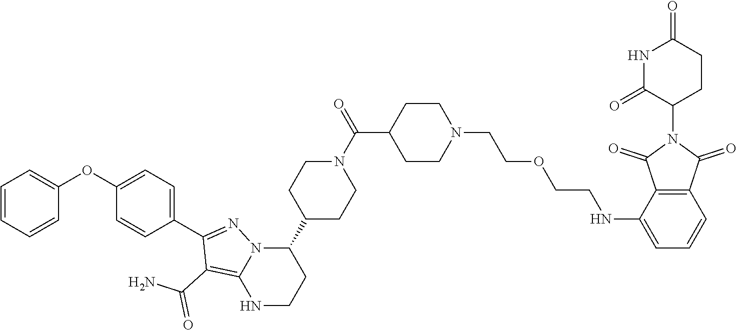 [SMILES: NC(=O)c1c(-c2ccc(Oc3ccccc3)cc2)nn2c1NCC[C@H]2C1CCN(C(=O)C2CCN(CCOCCNc3cccc4c3C(=O)N(C3CCC(=O)NC3=O)C4=O)CC2)CC1] | |
| 92 | 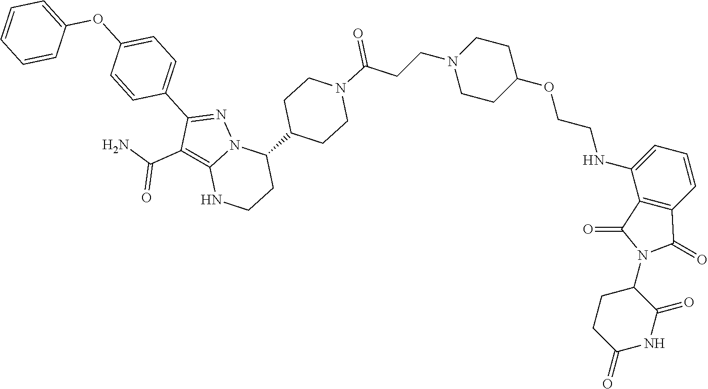 [SMILES: NC(=O)c1c(-c2ccc(Oc3ccccc3)cc2)nn2c1NCC[C@H]2C1CCN(C(=O)CCN2CCC(OCCNc3cccc4c3C(=O)N(C3CCC(=O)NC3=O)C4=O)CC2)CC1] | |
| 93 | 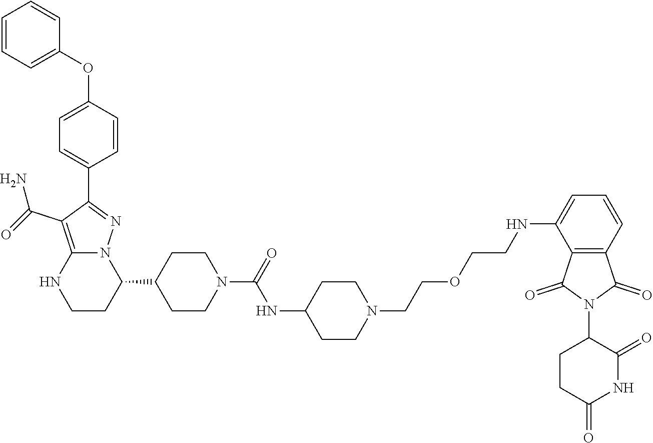 [SMILES: NC(=O)c1c(-c2ccc(Oc3ccccc3)cc2)nn2c1NCC[C@H]2C1CCN(C(=O)NC2CCN(CCOCCNc3cccc4c3C(=O)N(C3CCC(=O)NC3=O)C4=O)CC2)CC1] | |
| 94 | 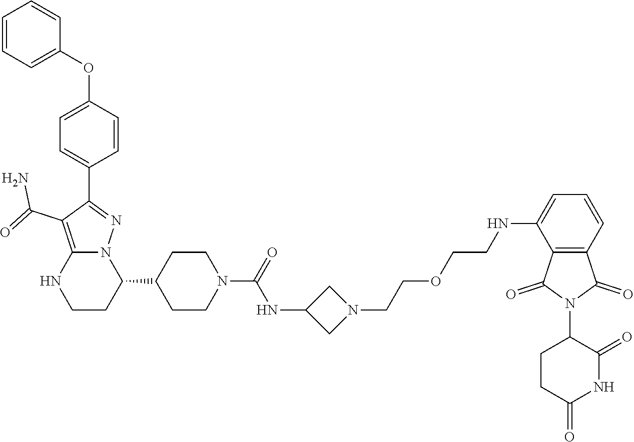 [SMILES: NC(=O)c1c(-c2ccc(Oc3ccccc3)cc2)nn2c1NCC[C@H]2C1CCN(C(=O)NC2CN(CCOCCNc3cccc4c3C(=O)N(C3CCC(=O)NC3=O)C4=O)C2)CC1] | |
| 95 | 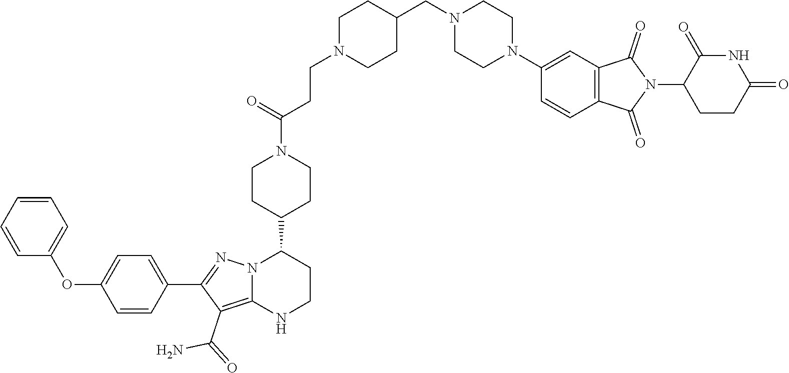 [SMILES: NC(=O)c1c(-c2ccc(Oc3ccccc3)cc2)nn2c1NCC[C@H]2C1CCN(C(=O)CCN2CCC(CN3CCN(c4ccc5c(c4)C(=O)N(C4CCC(=O)NC4=O)C5=O)CC3)CC2)CC1] | 1.68 |
| 96 | 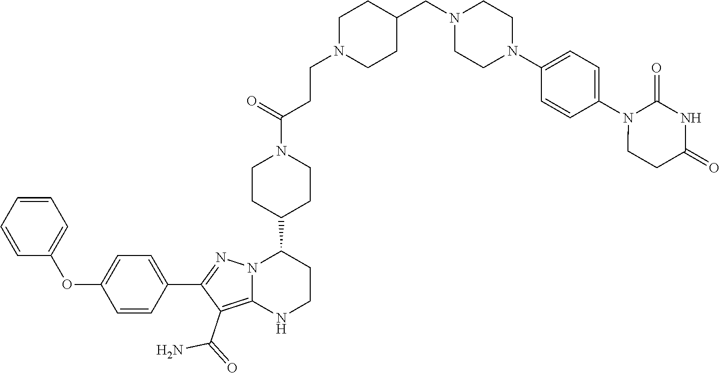 [SMILES: NC(=O)c1c(-c2ccc(Oc3ccccc3)cc2)nn2c1NCC[C@H]2C1CCN(C(=O)CCN2CCC(CN3CCN(c4ccc(N5CCC(=O)NC5=O)cc4)CC3)CC2)CC1] | |
| 97 | 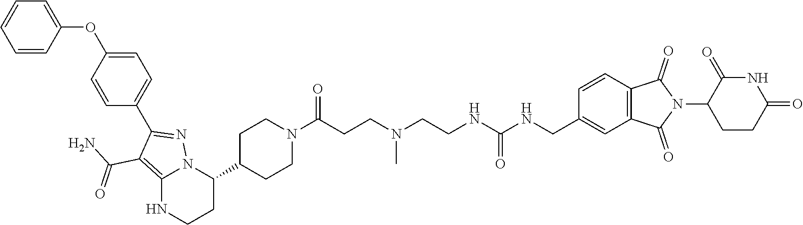 [SMILES: CN(CCNC(=O)NCc1ccc2c(c1)C(=O)N(C1CCC(=O)NC1=O)C2=O)CCC(=O)N1CCC([C@@H]2CCNc3c(C(N)=O)c(-c4ccc(Oc5ccccc5)cc4)nn32)CC1] | |
| 98 | 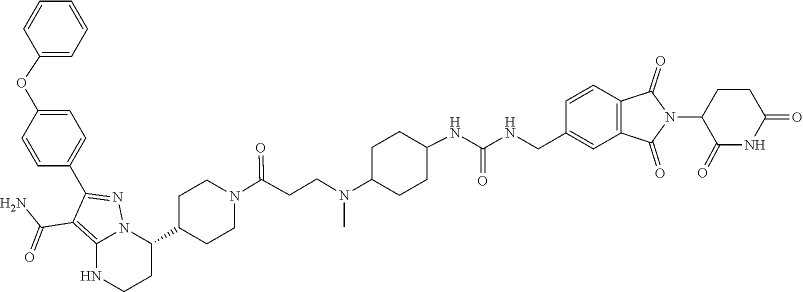 [SMILES: CN(CCC(=O)N1CCC([C@@H]2CCNc3c(C(N)=O)c(-c4ccc(Oc5ccccc5)cc4)nn32)CC1)C1CCC(NC(=O)NCc2ccc3c(c2)C(=O)N(C2CCC(=O)NC2=O)C3=O)CC1] | |
| 99 | 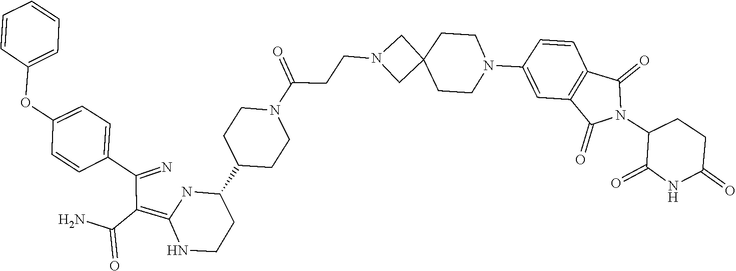 | |
| 100 | 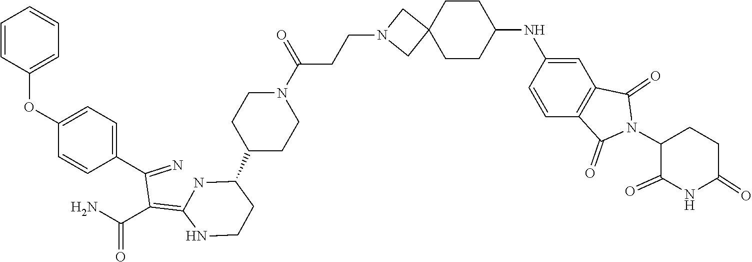 | |
| 101 | 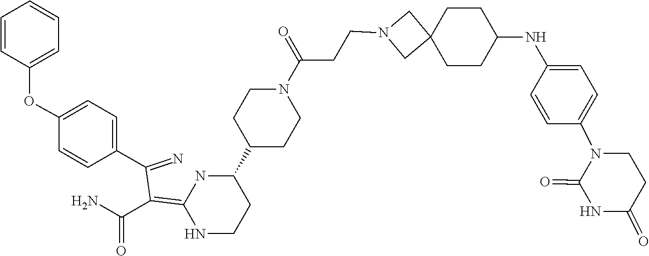 | |
| 102 | 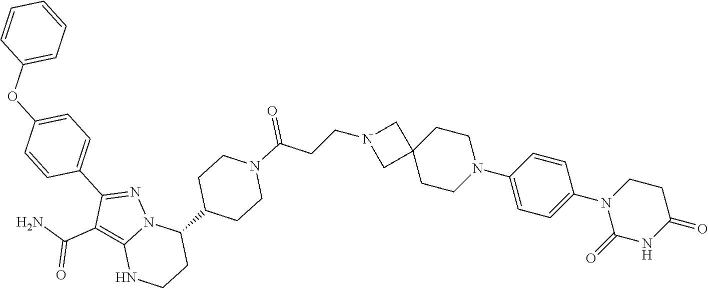 [SMILES: NC(=O)c1c(-c2ccc(Oc3ccccc3)cc2)nn2c1NCC[C@H]2C1CCN(C(=O)CCN2CC3(CCN(c4ccc(N5CCC(=O)NC5=O)cc4)CC3)C2)CC1] | |
| 103 | 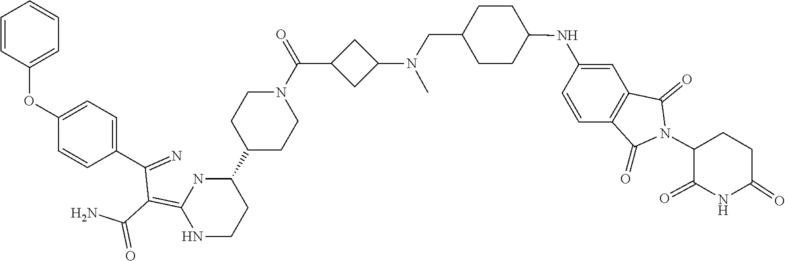 [SMILES: CN(CC1CCC(Nc2ccc3c(c2)C(=O)N(C2CCC(=O)NC2=O)C3=O)CC1)C1CC(C(=O)N2CCC([C@@H]3CCN/C(=C(/C(=N)c4ccc(Oc5ccccc5)cc4)C(N)=O)N3)CC2)C1] | |
| 104 | 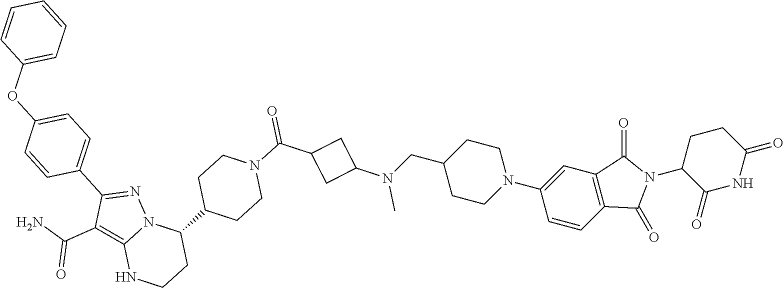 [SMILES: CN(CC1CCN(c2ccc3c(c2)C(=O)N(C2CCC(=O)NC2=O)C3=O)CC1)C1CC(C(=O)N2CCC([C@@H]3CCNc4c(C(N)=O)c(-c5ccc(Oc6ccccc6)cc5)nn43)CC2)C1] | |
| 105 | 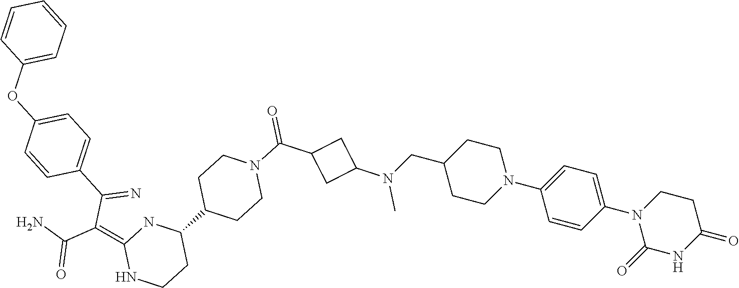 [SMILES: CN(CC1CCN(c2ccc(N3CCC(=O)NC3=O)cc2)CC1)C1CC(C(=O)N2CCC([C@@H]3CCN/C(=C(/C(=N)c4ccc(Oc5ccccc5)cc4)C(N)=O)N3)CC2)C1] | |
| 106 | 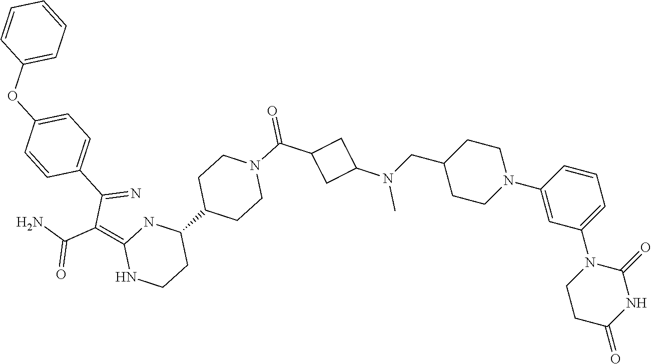 [SMILES: CN(CC1CCN(c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1] | |
| 107 | 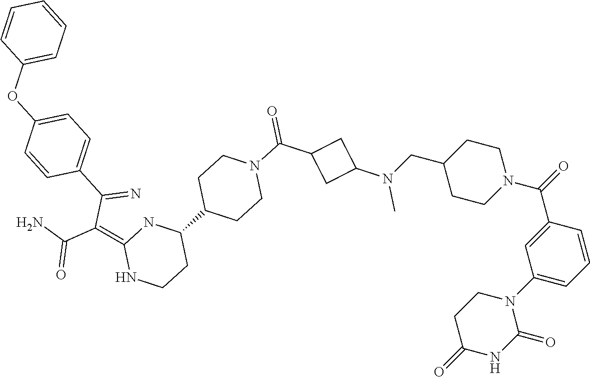 [SMILES: CN(CC1CCN(C(=O)c2cccc(N3CCC(=O)NC3=O)c2)CC1)C1CC(C(=O)N2CCC([C@@H]3CCN/C(=C(/C(=N)c4ccc(Oc5ccccc5)cc4)C(N)=O)N3)CC2)C1] | |
| 108 | 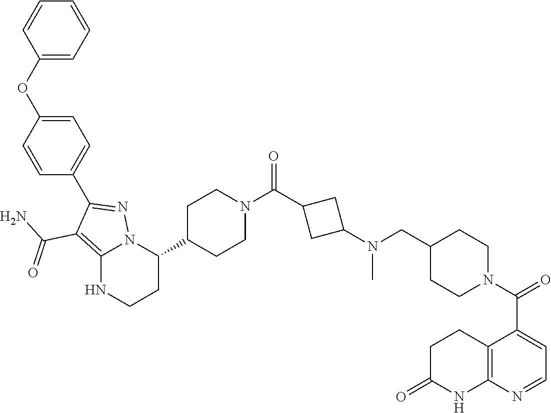 [SMILES: CN(CC1CCN(C(=O)c2ccnc3c2CCC(=O)N3)CC1)C1CC(C(=O)N2CCC([C@@H]3CCNc4c(C(N)=O)c(-c5ccc(Oc6ccccc6)cc5)nn43)CC2)C1] | |
| 109 | 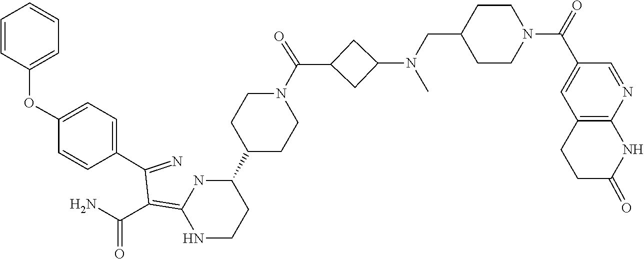 | |
| 110 | 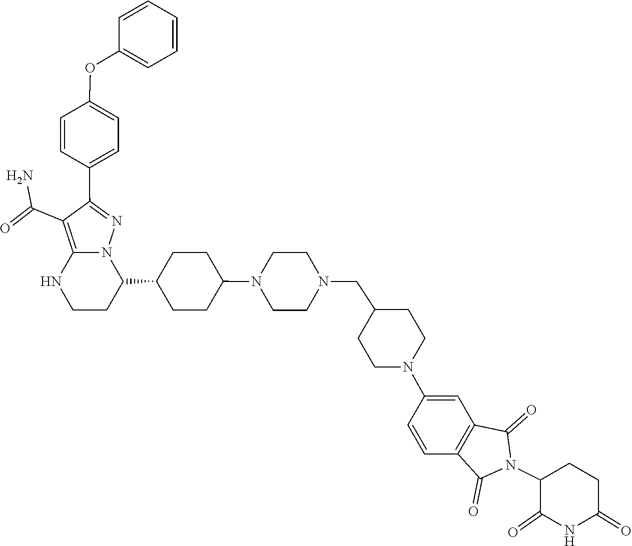 | |
| 111 | 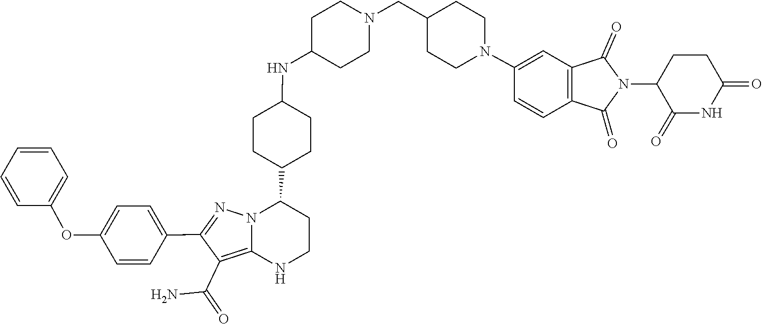 [SMILES: NC(=O)c1c(-c2ccc(Oc3ccccc3)cc2)nn2c1NCC[C@H]2C1CCC(NC2CCN(CC3CCN(c4ccc5c(c4)C(=O)N(C4CCC(=O)NC4=O)C5=O)CC3)CC2)CC1] | |
| 112 | 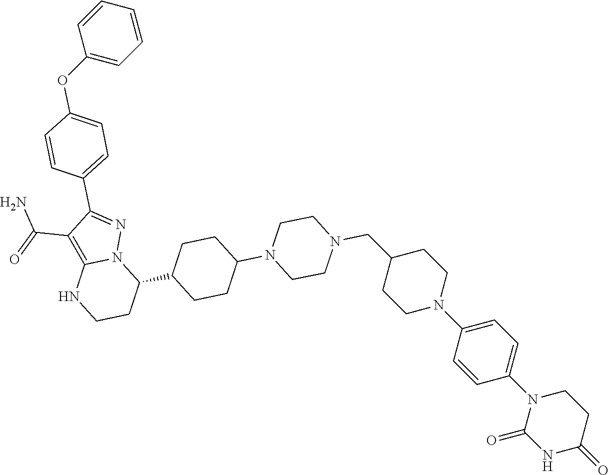 [SMILES: NC(=O)c1c(-c2ccc(Oc3ccccc3)cc2)nn2c1NCC[C@H]2C1CCC(N2CCN(CC3CCN(c4ccc(N5CCC(=O)NC5=O)cc4)CC3)CC2)CC1] | |
| 113 | 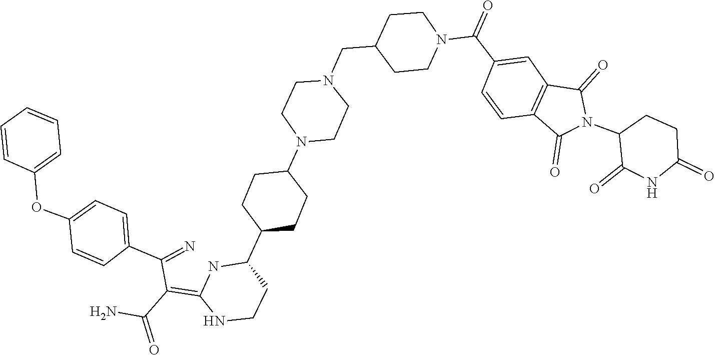 | |
| 114 | 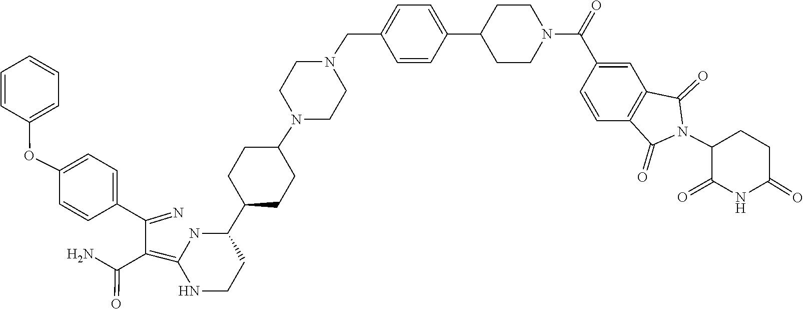 | |
| 115 | 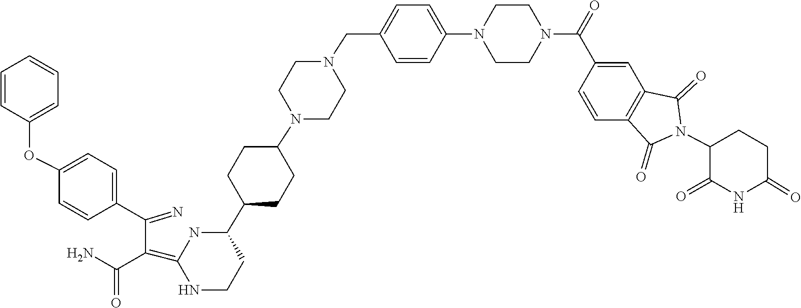 | |
| 116 | 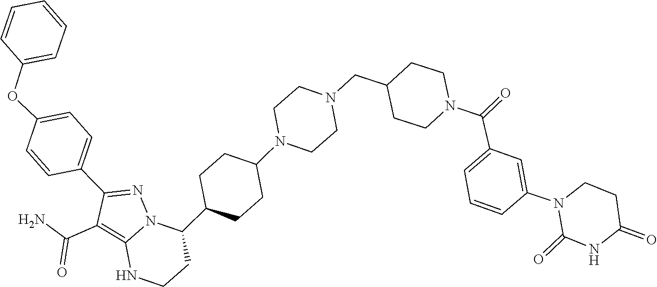 | |
| 117 | 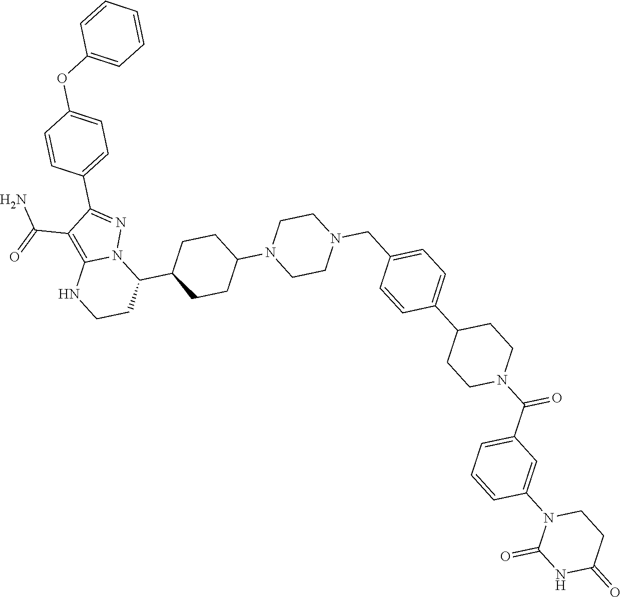 | |
| 118 | 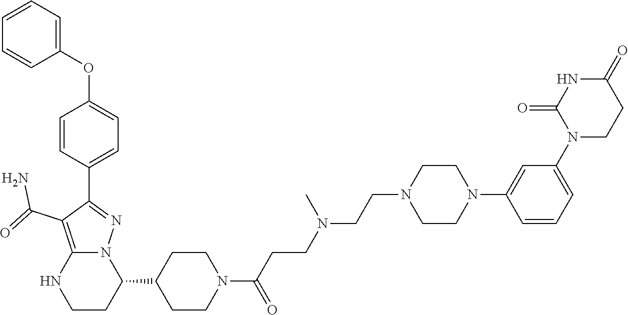 [SMILES: CN(CCC(=O)N1CCC([C@@H]2CCNc3c(C(N)=O)c(-c4ccc(Oc5ccccc5)cc4)nn32)CC1)CCN1CCN(c2cccc(N3CCC(=O)NC3=O)c2)CC1] | |
| 119 | 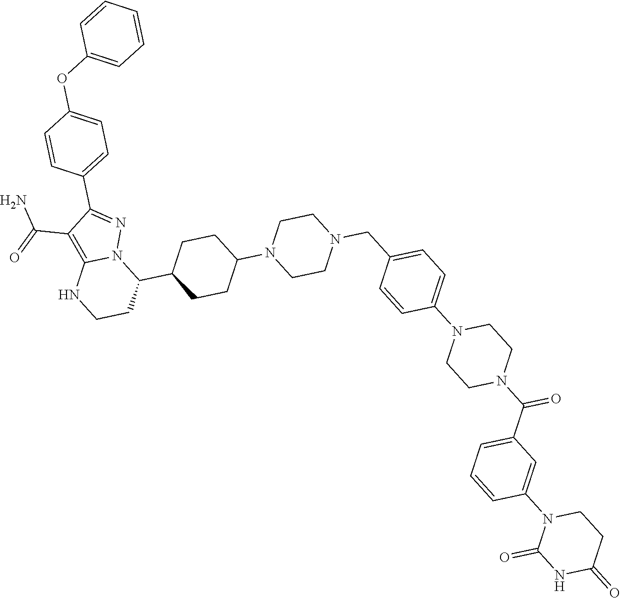 | |
| 120 | 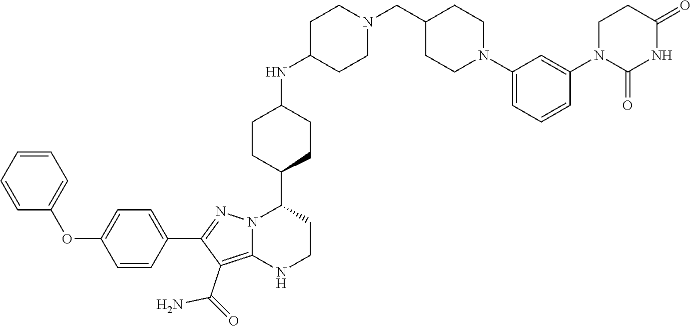 | |
| 121 | 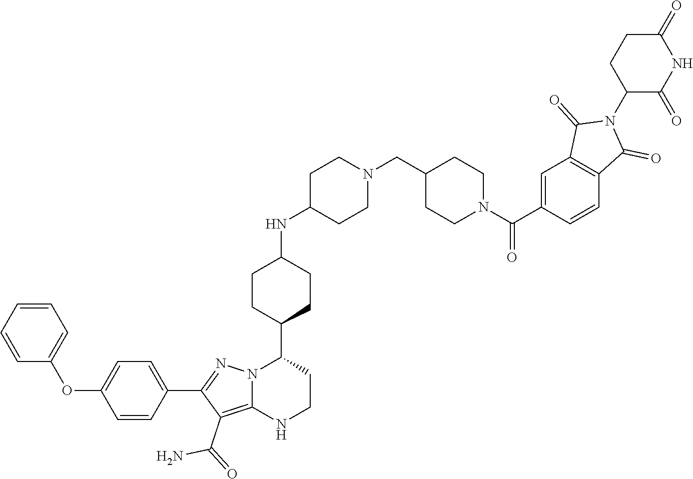 | |
| 122 | 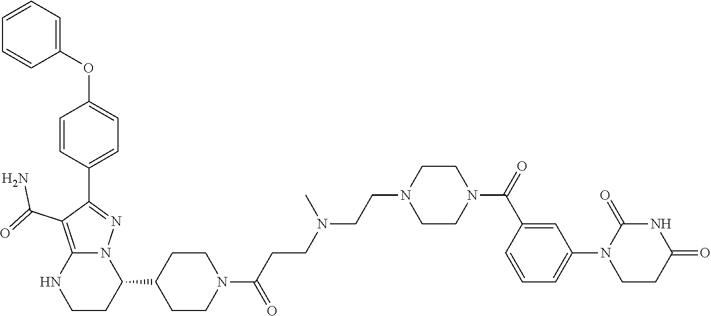 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cccc(N3CCC(=O)NC3=O)c2)CC1] | |
| 123 | 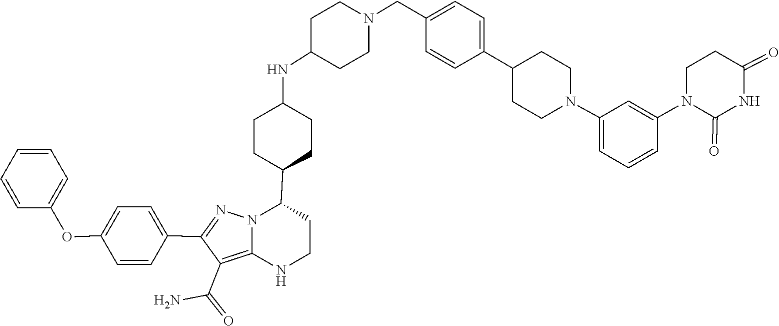 | |
| 124 | 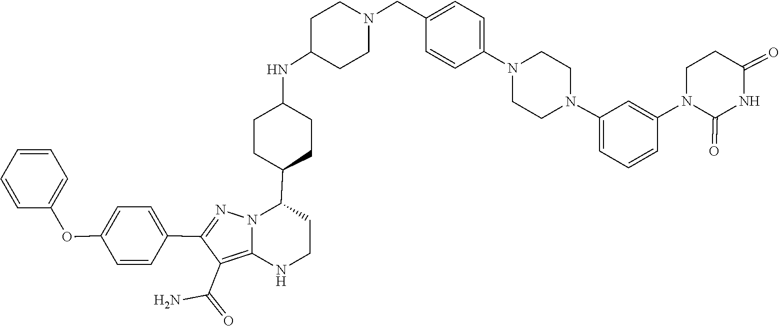 | |
| 125 | 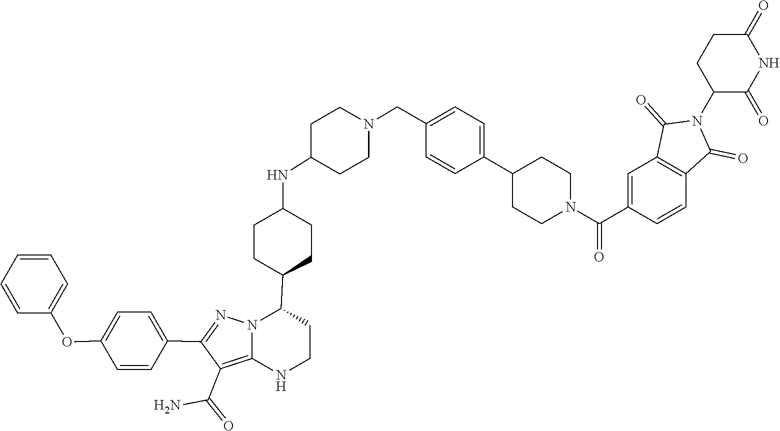 | |
| 126 | 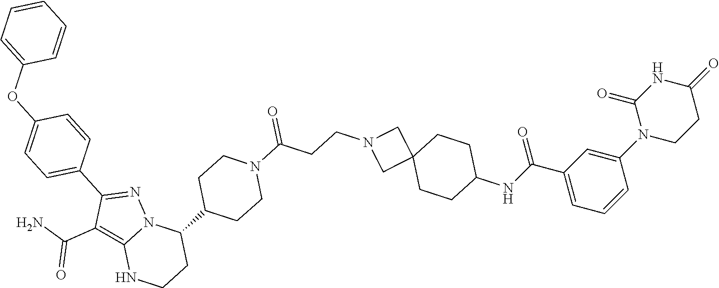 [SMILES: NC(=O)c1c(-c2ccc(Oc3ccccc3)cc2)nn2c1NCC[C@H]2C1CCN(C(=O)CCN2CC3(CCC(NC(=O)c4cccc(N5CCC(=O)NC5=O)c4)CC3)C2)CC1] | |
| 127 | 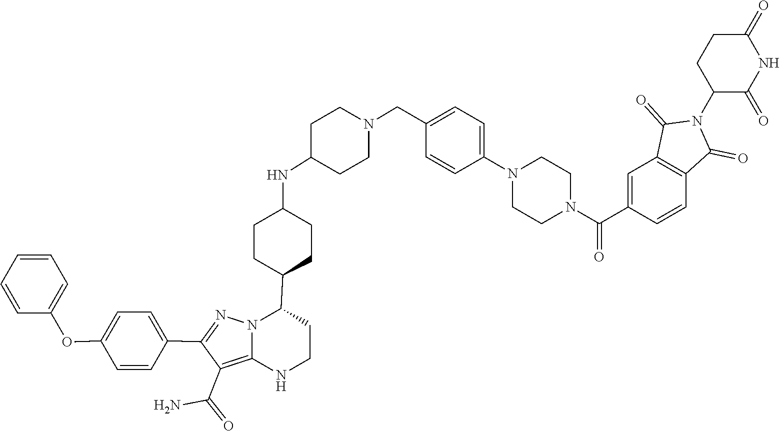 | |
| 128 | 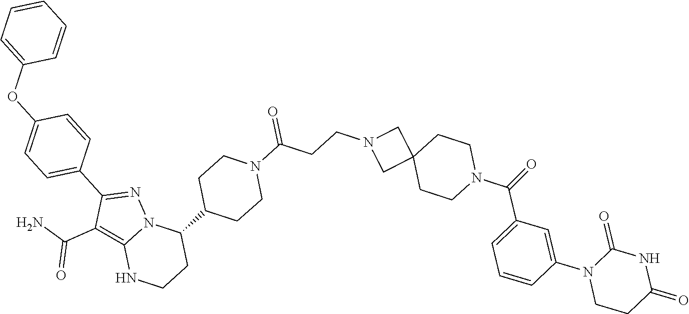 [SMILES: NC(=O)c1c(-c2ccc(Oc3ccccc3)cc2)nn2c1NCC[C@H]2C1CCN(C(=O)CCN2CC3(CCN(C(=O)c4cccc(N5CCC(=O)NC5=O)c4)CC3)C2)CC1] | |
| 129 | 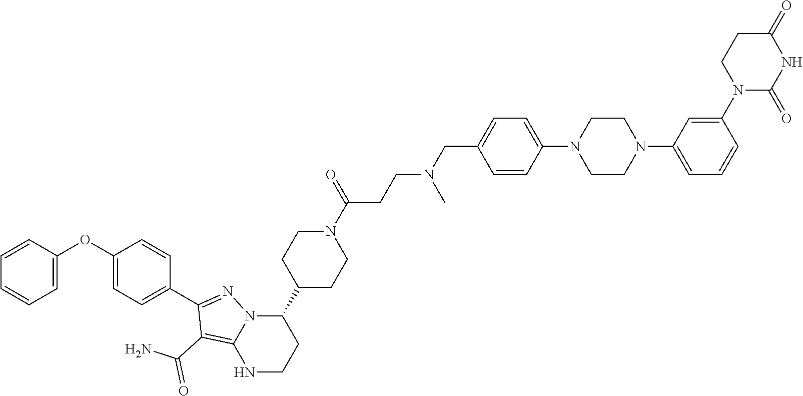 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3cccc(N4CCC(=O)NC4=O)c3)CC2)cc1] | |
| 130 | 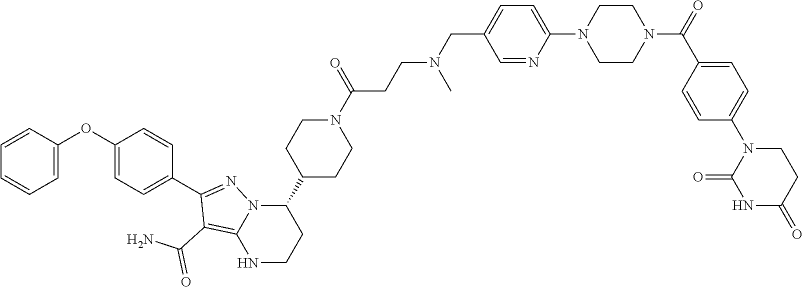 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)nc1] | |
| 131 | 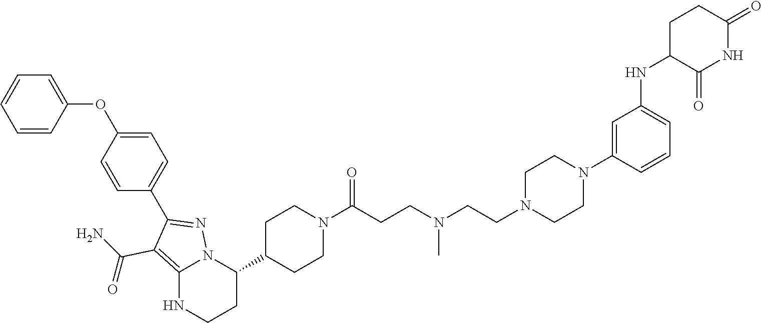 [SMILES: CN(CCC(=O)N1CCC([C@@H]2CCNc3c(C(N)=O)c(-c4ccc(Oc5ccccc5)cc4)nn32)CC1)CCN1CCN(c2cccc(NC3CCC(=O)NC3=O)c2)CC1] | |
| 132 | 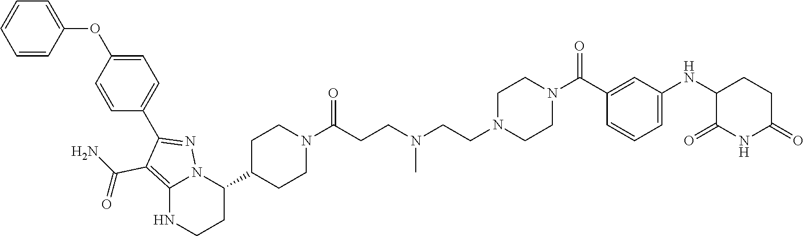 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cccc(NC3CCC(=O)NC3=O)c2)CC1] | |
| 133 | 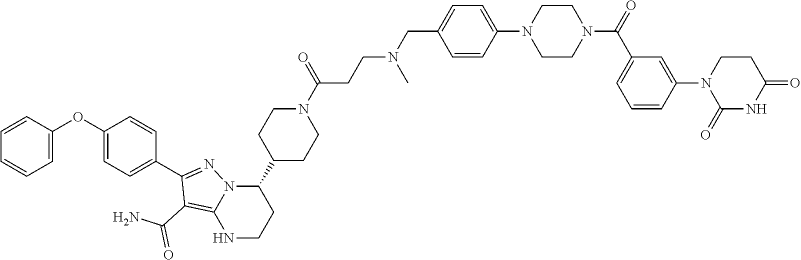 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cc1] | |
| 134 | 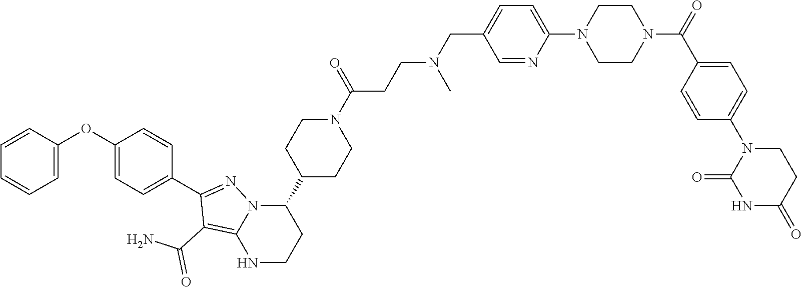 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)nc1] | |
| 135 | 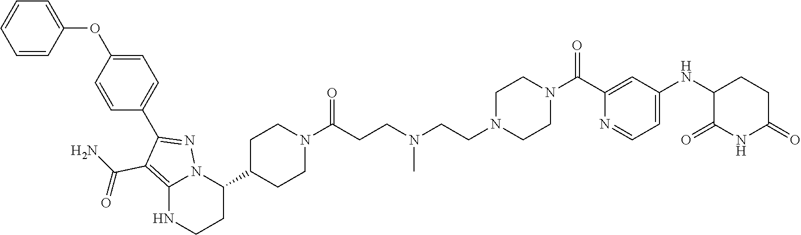 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccn2)CC1] | |
| 136 | 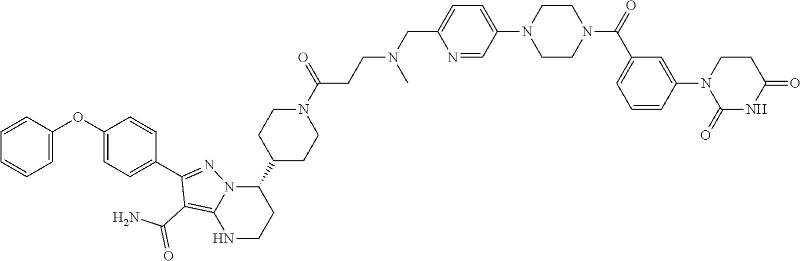 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cccc(N4CCC(=O)NC4=O)c3)CC2)cn1] | |
| 137 | 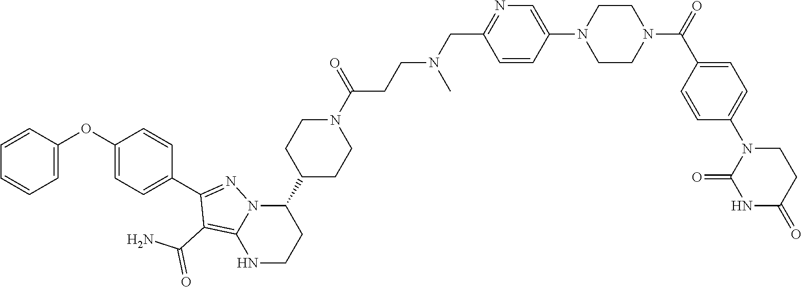 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3ccc(N4CCC(=O)NC4=O)cc3)CC2)cn1] | |
| 138 | 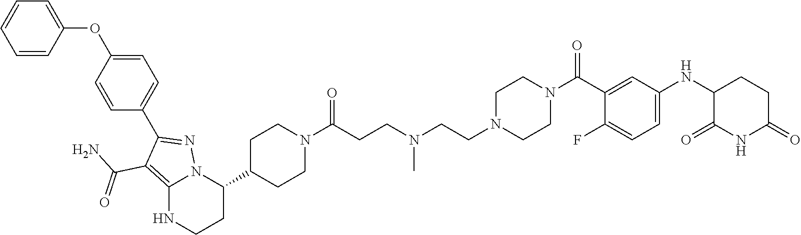 [SMILES: CN(CCC(=O)N1CCC([C@@H]2CCNc3c(C(N)=O)c(-c4ccc(Oc5ccccc5)cc4)nn32)CC1)CCN1CCN(C(=O)c2cc(NC3CCC(=O)NC3=O)ccc2F)CC1] | |
| 139 | 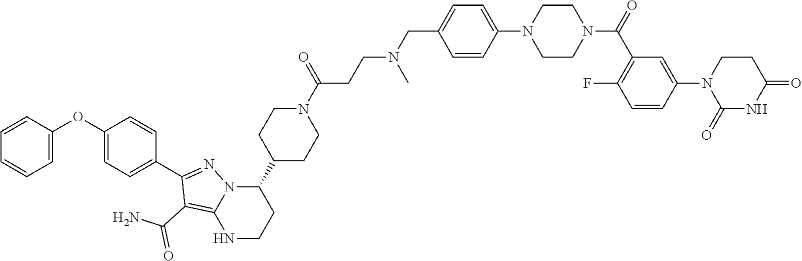 [SMILES: CN(CCC(=O)N1CCC([C@@H]2CCNc3c(C(N)=O)c(-c4ccc(Oc5ccccc5)cc4)nn32)CC1)Cc1ccc(N2CCN(C(=O)c3cc(N4CCC(=O)NC4=O)ccc3F)CC2)cc1] | |
| 140 | 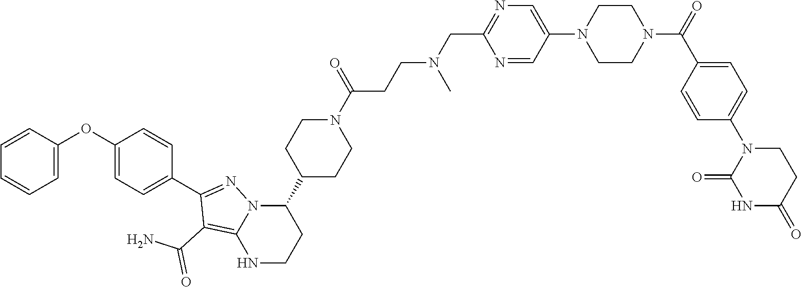 [SMILES: CN(CCC(=O)N1CCC([C@@H]2CCNc3c(C(N)=O)c(-c4ccc(Oc5ccccc5)cc4)nn32)CC1)Cc1ncc(N2CCN(C(=O)c3ccc(N4CCC(=O)NC4=O)cc3)CC2)cn1] | |
| 141 | 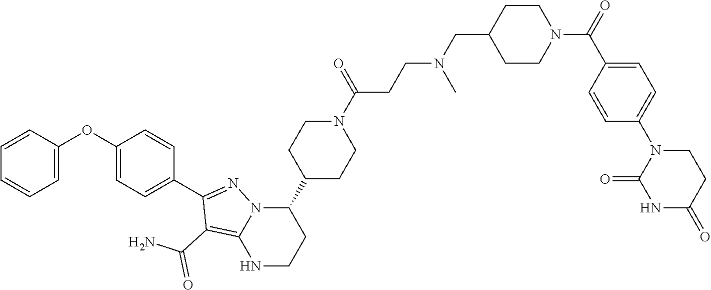 [SMILES: CN(CCC(=O)N1CCC([C@@H]2CCNc3c(C(N)=O)c(-c4ccc(Oc5ccccc5)cc4)nn32)CC1)CC1CCN(C(=O)c2ccc(N3CCC(=O)NC3=O)cc2)CC1] | |
| 142 | 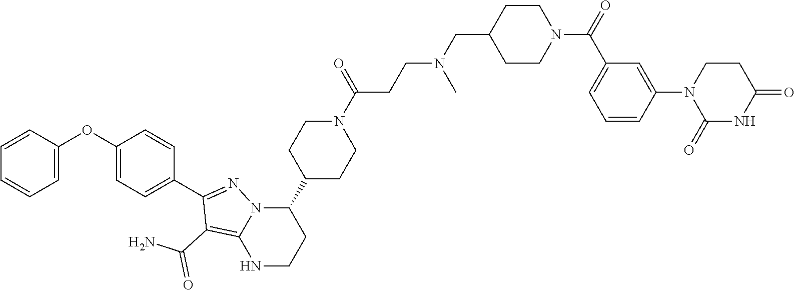 [SMILES: CN(CCC(=O)N1CCC([C@@H]2CCNc3c(C(N)=O)c(-c4ccc(Oc5ccccc5)cc4)nn32)CC1)CC1CCN(C(=O)c2cccc(N3CCC(=O)NC3=O)c2)CC1] | |
| 143 | 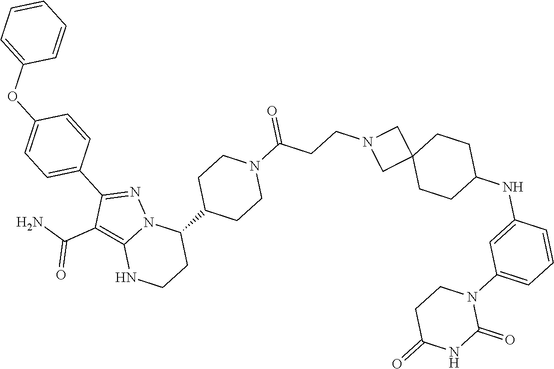 [SMILES: NC(=O)c1c(-c2ccc(Oc3ccccc3)cc2)nn2c1NCC[C@H]2C1CCN(C(=O)CCN2CC3(CCC(Nc4cccc(N5CCC(=O)NC5=O)c4)CC3)C2)CC1] | |
| 144 | 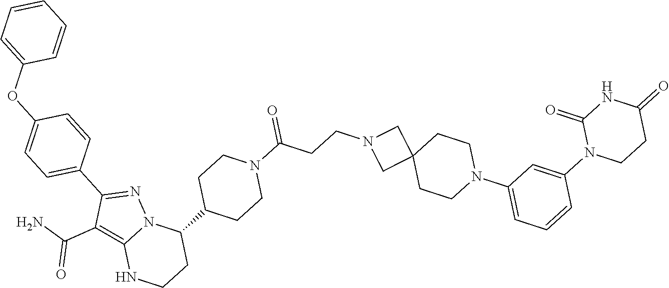 [SMILES: NC(=O)c1c(-c2ccc(Oc3ccccc3)cc2)nn2c1NCC[C@H]2C1CCN(C(=O)CCN2CC3(CCN(c4cccc(N5CCC(=O)NC5=O)c4)CC3)C2)CC1] | |
| 145 | 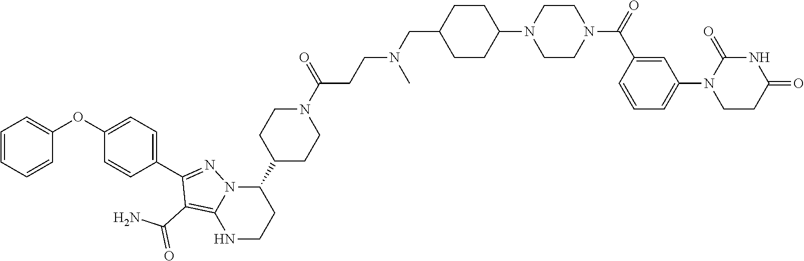 [SMILES: CN(CCC(=O)N1CCC([C@@H]2CCNc3c(C(N)=O)c(-c4ccc(Oc5ccccc5)cc4)nn32)CC1)CC1CCC(N2CCN(C(=O)c3cccc(N4CCC(=O)NC4=O)c3)CC2)CC1] | |
| 146 | 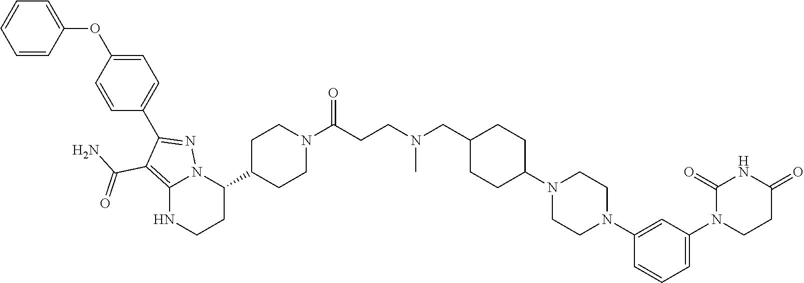 [SMILES: CN(CCC(=O)N1CCC([C@@H]2CCNc3c(C(N)=O)c(-c4ccc(Oc5ccccc5)cc4)nn32)CC1)CC1CCC(N2CCN(c3cccc(N4CCC(=O)NC4=O)c3)CC2)CC1] | |
| 147 | 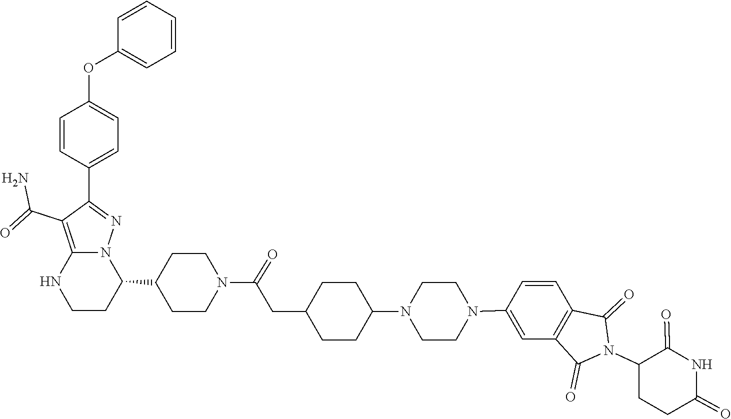 [SMILES: NC(=O)c1c(-c2ccc(Oc3ccccc3)cc2)nn2c1NCC[C@H]2C1CCN(C(=O)CC2CCC(N3CCN(c4ccc5c(c4)C(=O)N(C4CCC(=O)NC4=O)C5=O)CC3)CC2)CC1] | 0.244 |
| 148 | 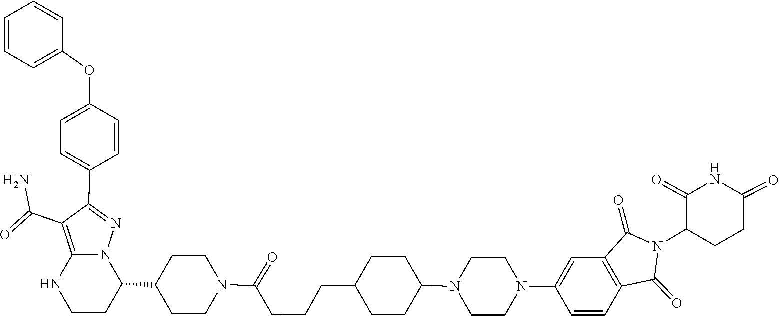 [SMILES: NC(=O)c1c(-c2ccc(Oc3ccccc3)cc2)nn2c1NCC[C@H]2C1CCN(C(=O)CCCC2CCC(N3CCN(c4ccc5c(c4)C(=O)N(C4CCC(=O)NC4=O)C5=O)CC3)CC2)CC1] | 0.205 |
| 149 | 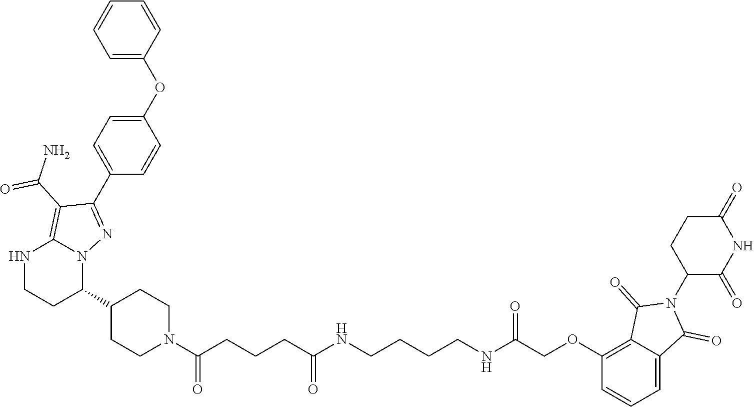 [SMILES: NC(=O)c1c(-c2ccc(Oc3ccccc3)cc2)nn2c1NCC[C@H]2C1CCN(C(=O)CCCC(=O)NCCCCNC(=O)COc2cccc3c2C(=O)N(C2CCC(=O)NC2=O)C3=O)CC1] | |
| 150 | 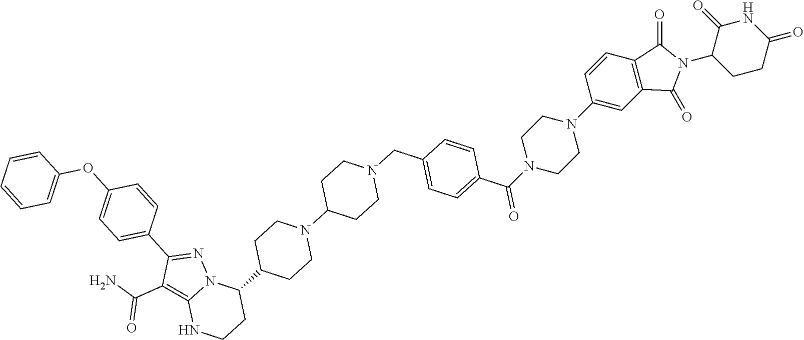 [SMILES: NC(=O)c1c(-c2ccc(Oc3ccccc3)cc2)nn2c1NCC[C@H]2C1CCN(C2CCN(Cc3ccc(C(=O)N4CCN(c5ccc6c(c5)C(=O)N(C5CCC(=O)NC5=O)C6=O)CC4)cc3)CC2)CC1] | |
| 151 | 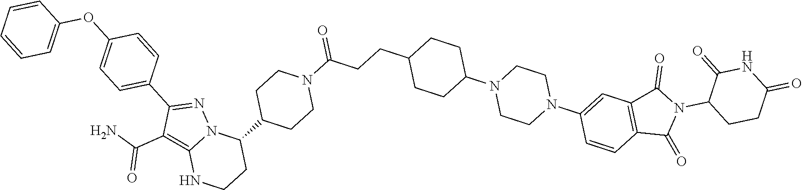 [SMILES: NC(=O)c1c(-c2ccc(Oc3ccccc3)cc2)nn2c1NCC[C@H]2C1CCN(C(=O)CCC2CCC(N3CCN(c4ccc5c(c4)C(=O)N(C4CCC(=O)NC4=O)C5=O)CC3)CC2)CC1] | 0.017 |
| 152 | 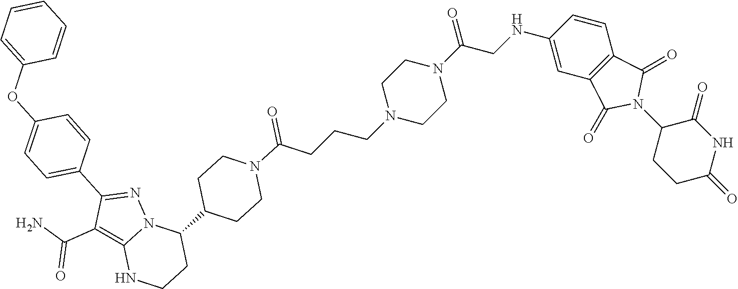 [SMILES: NC(=O)c1c(-c2ccc(Oc3ccccc3)cc2)nn2c1NCC[C@H]2C1CCN(C(=O)CCCN2CCN(C(=O)CNc3ccc4c(c3)C(=O)N(C3CCC(=O)NC3=O)C4=O)CC2)CC1] | 0.035 |
| 153 | 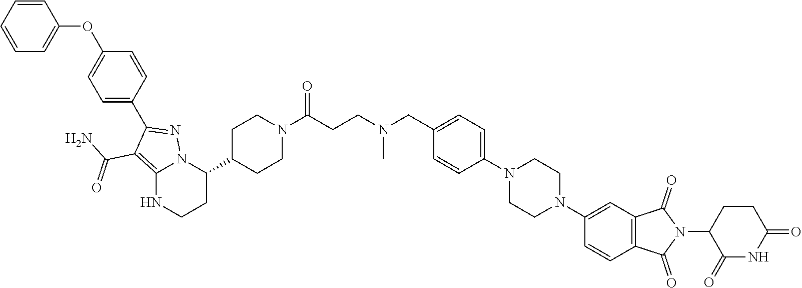 [SMILES: CN(CCC(=O)N1CCC([C@@H]2CCNc3c(C(N)=O)c(-c4ccc(Oc5ccccc5)cc4)nn32)CC1)Cc1ccc(N2CCN(c3ccc4c(c3)C(=O)N(C3CCC(=O)NC3=O)C4=O)CC2)cc1] | |
| 154 | 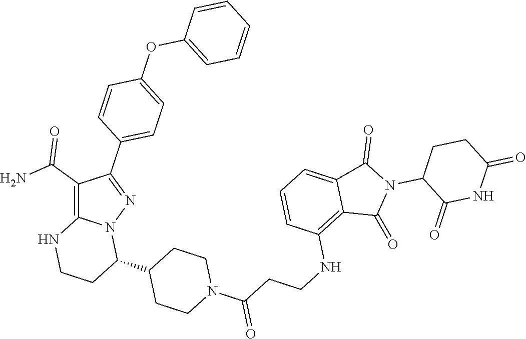 [SMILES: NC(=O)c1c(-c2ccc(Oc3ccccc3)cc2)nn2c1NCC[C@H]2C1CCN(C(=O)CCNc2cccc3c2C(=O)N(C2CCC(=O)NC2=O)C3=O)CC1] | 0.637 |
| 155 | 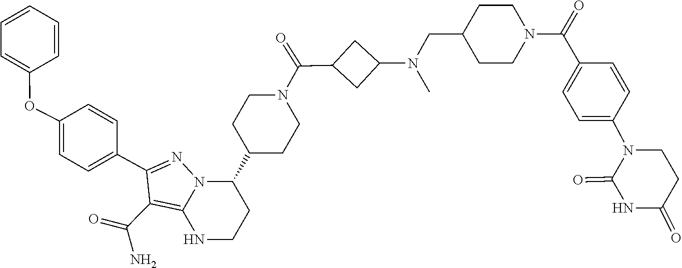 [SMILES: CN(CC1CCN(C(=O)c2ccc(N3CCC(=O)NC3=O)cc2)CC1)C1CC(C(=O)N2CCC([C@@H]3CCNc4c(C(N)=O)c(-c5ccc(Oc6ccccc6)cc5)nn43)CC2)C1] | 0.0072 |
| 156 | 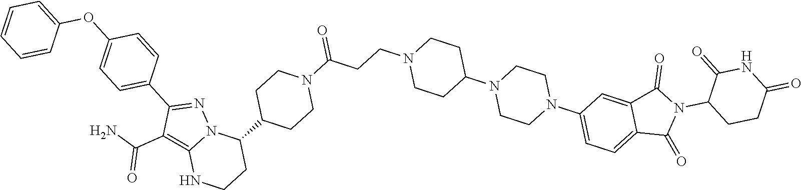 [SMILES: NC(=O)c1c(-c2ccc(Oc3ccccc3)cc2)nn2c1NCC[C@H]2C1CCN(C(=O)CCN2CCC(N3CCN(c4ccc5c(c4)C(=O)N(C4CCC(=O)NC4=O)C5=O)CC3)CC2)CC1] | 1.68 |
| 157 | 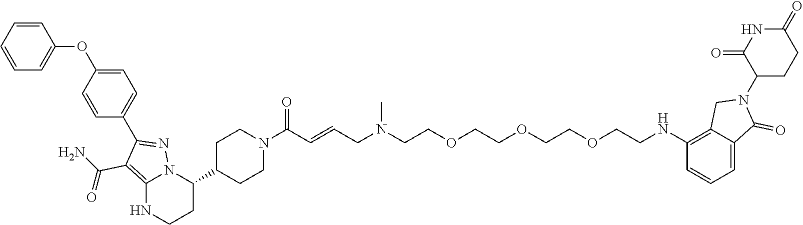 [SMILES: CN(C/C=C/C(=O)N1CCC([C@@H]2CCNc3c(C(N)=O)c(-c4ccc(Oc5ccccc5)cc4)nn32)CC1)CCOCCOCCOCCNc1cccc2c1CN(C1CCC(=O)NC1=O)C2=O] | |
| 158 | 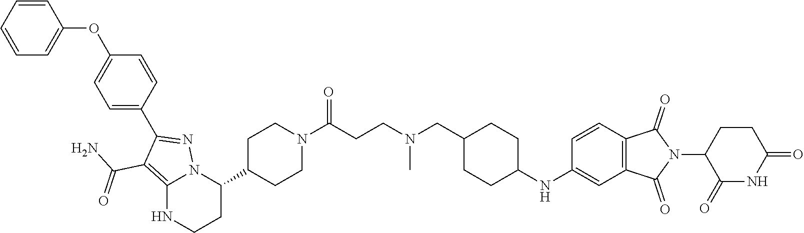 [SMILES: CN(CCC(=O)N1CCC([C@@H]2CCNc3c(C(N)=O)c(-c4ccc(Oc5ccccc5)cc4)nn32)CC1)CC1CCC(Nc2ccc3c(c2)C(=O)N(C2CCC(=O)NC2=O)C3=O)CC1] | |
| 159 | 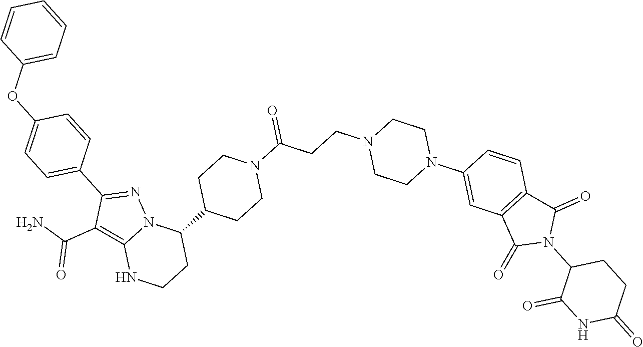 [SMILES: NC(=O)c1c(-c2ccc(Oc3ccccc3)cc2)nn2c1NCC[C@H]2C1CCN(C(=O)CCN2CCN(c3ccc4c(c3)C(=O)N(C3CCC(=O)NC3=O)C4=O)CC2)CC1] | |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 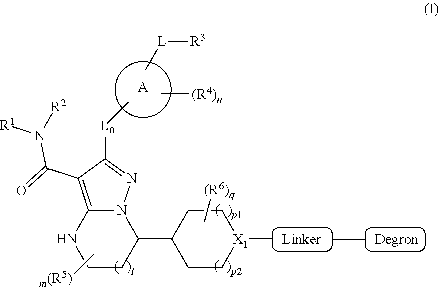
or a pharmaceutically acceptable salt thereof, or a stereoisomer thereof,
wherein:
A is a 5- or 6-membered aromatic ring comprising 0-3 heteroatoms selected from N, S and O;
L and L₀ are each independently a bond, —CH₂—, —NR⁷—, —O—, or —S—;
m, n and q are each independently 0, 1, 2, 3 or 4;
t is 0, 1 or 2;
p1 and p2 are each independently 0, 1 or 2;
R¹, R², and R⁷ are each independently hydrogen, —C₁₋₈alkyl, —C₂₋₈ alkenyl, —C₂₋₈ alkynyl, cycloalkyl, heterocyclyl, aryl, or heteroaryl, each of said —C₁₋₈ alkyl, —C₂₋₈ alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R³, R⁴, R⁵, and R⁶ are each independently hydrogen, halogen, —C₁₋₈alkyl, —C₂₋₈ alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{a}, R^{b}, and R^{c} are each independently hydrogen, —C₁₋₈alkyl, —C₂₋₈alkenyl, —C₂₋₈alkynyl, cycloalkyl, heterocyclyl, aryl, or heteroaryl;
or R¹ and R², together with the nitrogen atom to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈ alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said —C₁₋₈alkyl, —C₂₋₈alkenyl, —C₂₋₈ alkynyl, cycloalkyl, heterocyclyl, aryl, or heteroaryl is optionally substituted with at least one substituent selected from halogen, —C₁₋₈ alkyl, —OR³ⁱ, —NR³ⁱR^{3j}, cycloalkyl, heterocyclyl, aryl, or heteroaryl;
R^{3f}, R^{3g}, R^{3h}, R³ⁱ, and R^{3j} are each independently hydrogen, —C₁₋₈alkyl, C₁₋₈alkoxy-C₁₋₈alkyl-, —C₂₋₈alkenyl, —C₂₋₈ alkynyl, cycloalkyl, heterocyclyl, aryl, or heteroaryl;
X₁ is selected from —CH— or N;
the Linker is a bond or a divalent linking group, and
the Degron is an E3 Ubiquitin ligase moiety.

2. The compound according to claim 1, wherein the Degron moiety is selected from Formulas D1, D2, D3, D4 or D5: 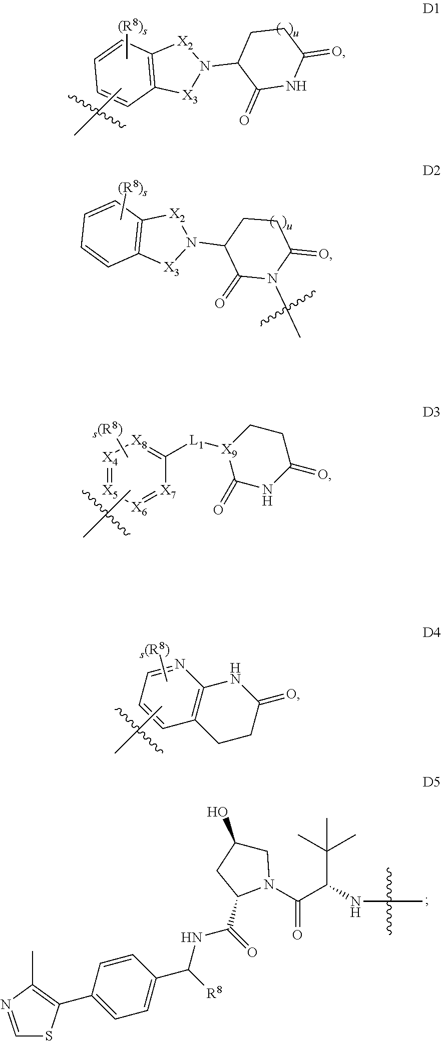
wherein
X₂ and X₃ are each independently —CH₂—, —NH— or —C(O)—;
X₄, X₅, X₆, X₇ and X₈ are each independently CH or N;
X₉ is CH or N;
L₁ is selected from a bond, —CH₂—, —O—, —NH— and —S—;
s is 0, 1, 2, 3, or 4;
u is 0, 1, or 2;
R⁸ is each independently hydrogen, halogen, —C₁₋₈alkyl, —C₂₋₈ alkenyl, —C₂₋₈ alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a}COR^{8b}, —NR^{8a}CONR^{8b}R^{8c}, —NR^{8a}, —CO₂R^{8b}, —NR^{8a}SONR^{8b}R^{8c}, —NR^{8a}SO₂NR^{8b}R^{8c}, or —NR^{8a}SO₂R^{8b}, each of said —C₁₋₈alkyl, —C₂₋₈ alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl;
wherein the Degron moiety binds to the linker via

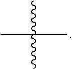

3. The compound according to claim 1, wherein Formula D1 is selected from

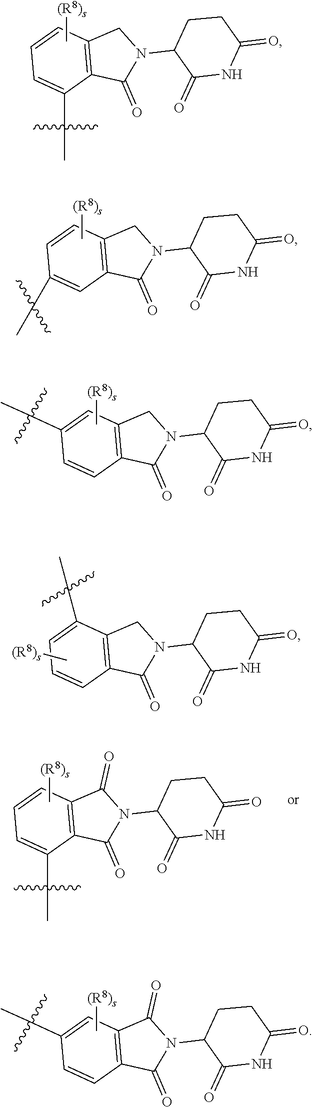

4. The compound according to claim 1, wherein Formula D2 is selected from

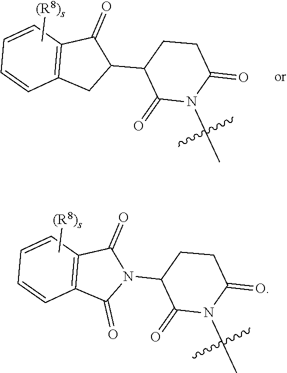

5. The compound according to claim 4, wherein Formula D2 is selected from

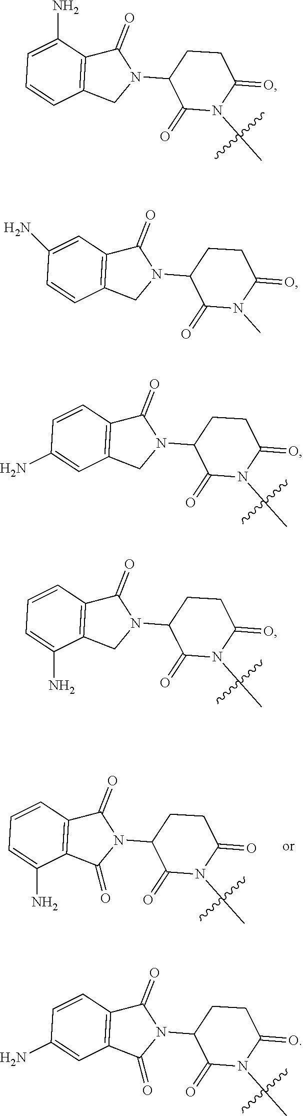 [SMILES: *N1C(=O)CCC(N2Cc3cccc(N)c3C2=O)C1=O ; CN1C(=O)CCC(N2Cc3ccc(N)cc3C2=O)C1=O ; *N1C(=O)CCC(N2Cc3cc(N)ccc3C2=O)C1=O ; *N1C(=O)CCC(N2Cc3c(N)cccc3C2=O)C1=O ; *N1C(=O)CCC(N2C(=O)c3cccc(N)c3C2=O)C1=O ; *N1C(=O)CCC(N2C(=O)c3ccc(N)cc3C2=O)C1=O]

6. The compound according to claim 2, wherein Formula D3 is selected from 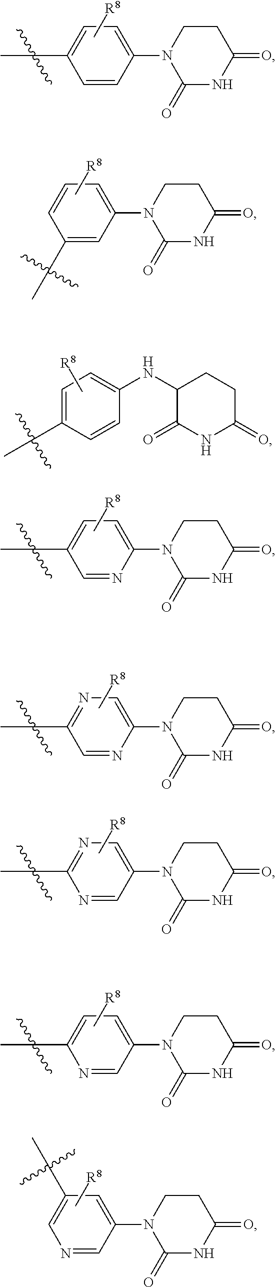 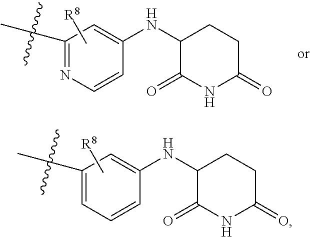
wherein R¹ is defined as above.

7. The compound according to claim 6, wherein Formula D3 is selected from

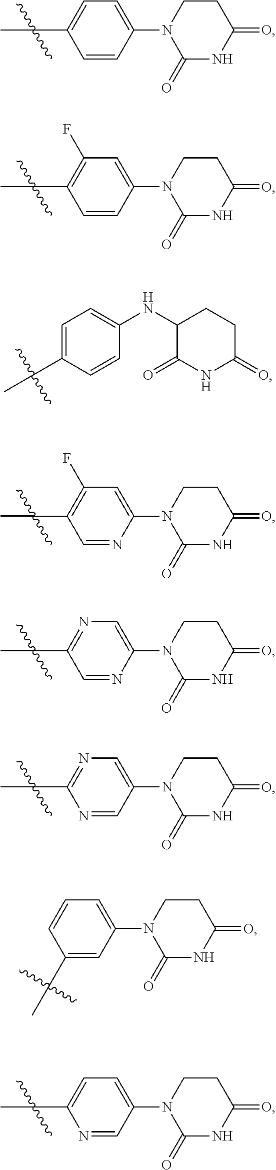

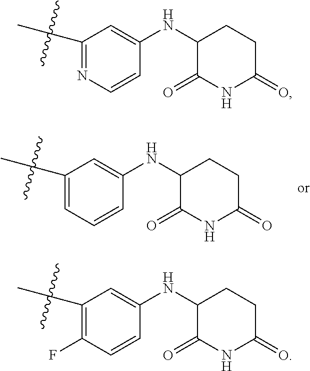 [SMILES: *c1cc(NC2CCC(=O)NC2=O)ccn1 ; *c1cccc(NC2CCC(=O)NC2=O)c1 ; *c1cc(NC2CCC(=O)NC2=O)ccc1F]

8. The compound according to claim 2, wherein Formula D4 is selected from

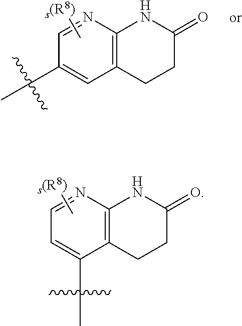

9. The compound according to claim 8, wherein Formula D4 is selected from

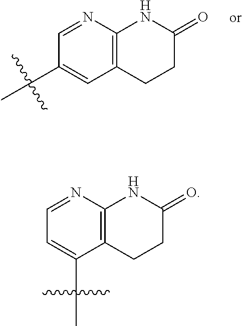 [SMILES: *c1cnc2c(c1)CCC(=O)N2 ; *c1ccnc2c1CCC(=O)N2]

10. The compound according to any one of claims 1-9, wherein A is phenyl; p=1, q=0, m=0, and t=1.

11. The compound according to any one of claims 1-10, wherein L is O or NH.

12. The compound according to any one of claims 1-11, wherein R¹ and R² are both H.

13. The compound according to any one of claims 1-11, wherein

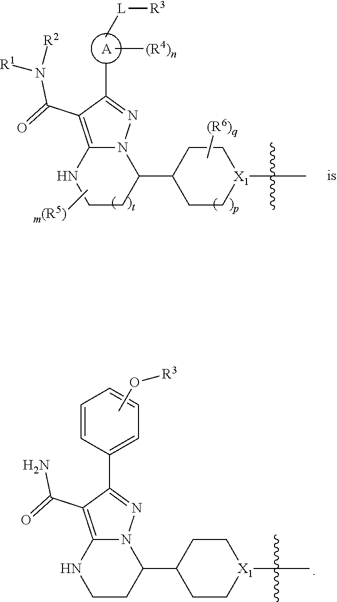

14. The compound according to claim 13, wherein

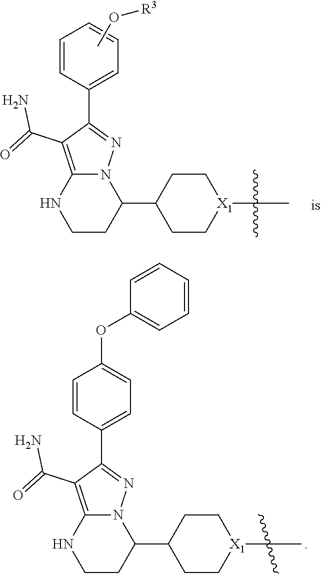

15. The compound according to claim 14, wherein

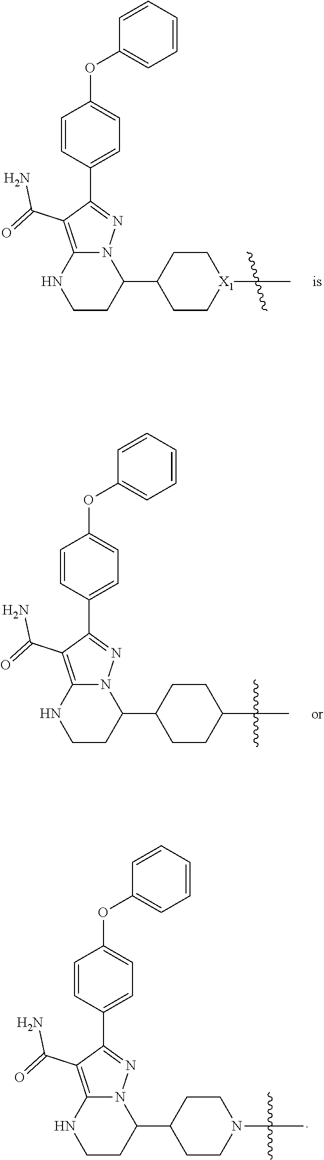

16. The compound according to claim 15, wherein

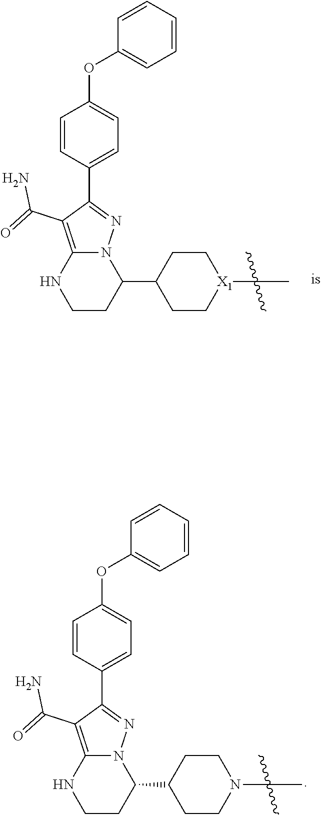

17. The compound according to any one of claims 1-16, wherein the Linker is selected from 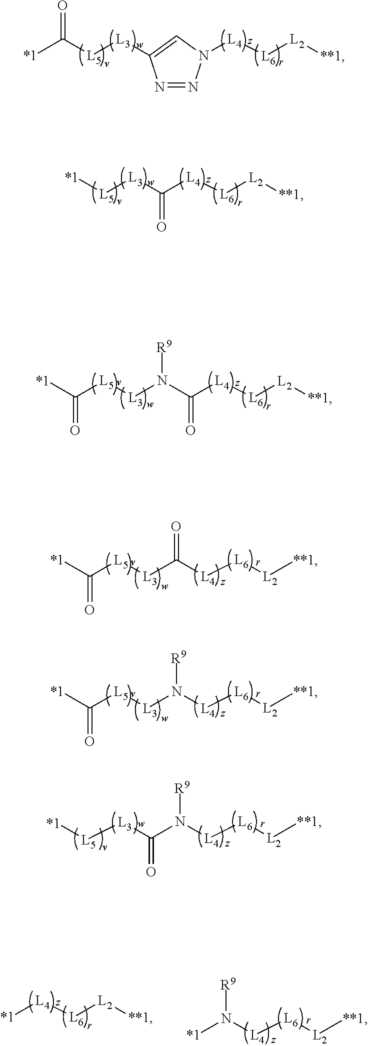
wherein *1 refers to the position attached to 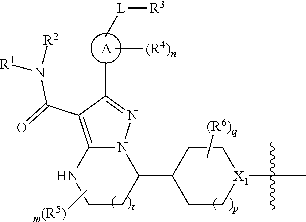
(sometimes referred to as the BTK moiety), and **1 refers to the position attached to the Degron;
r, v, w, and z are each independently 0, 1, 2, 3, 4, 5, 6, 7, 8, 9 or 10;
L₂ is —CH₂—, —NH—, —O—, —C(O)—, —NHC(O)—, 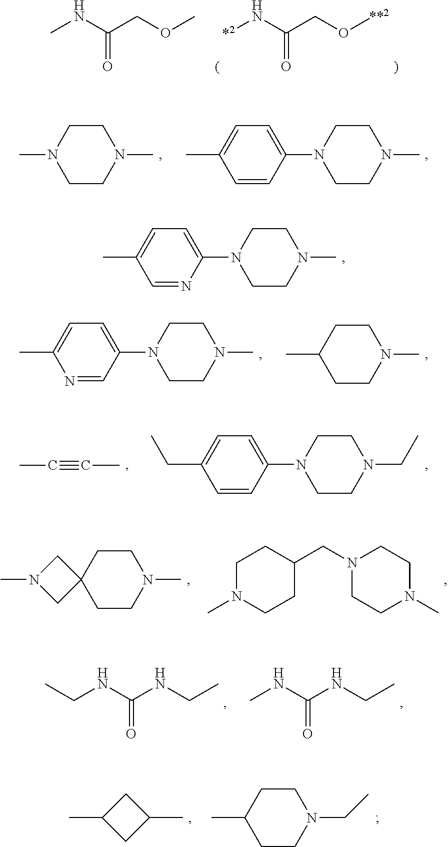
wherein *2 refers to the position attached to L₄ and **2 refers to the position attached to the Degron;
L₃, L₄, L₅ and L₆ are each independently —CH₂—, —CH₂CH₂—, —OCH₂CH₂—, —CH₂—O—CH₂—, —CH₂CH₂O—, —C(O)—, —NHC(O)—, —CH₂—CONH—, 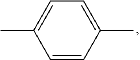
—CH═CH—, 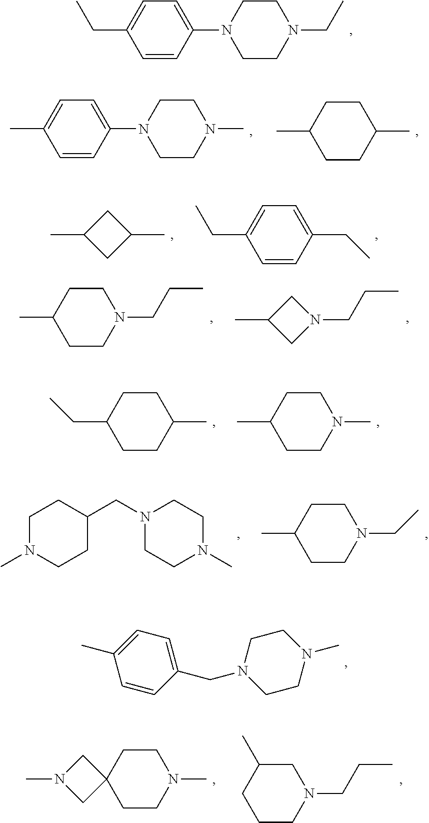
R⁹ is selected from H or CH₃.

18. The compound according to any one of claims 1-17, wherein the Linker is selected from 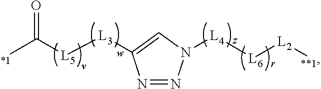
v=0; w=0, 1, 2, 3, or 4; L₃ is —CH₂—; L₄ is —CH₂CH₂O— or —CH₂—; z=0, 1, 2, 3, 4, 5, 6, or 7; L₆ is —CH₂— or —NHC(O)—; r=0, 1, 2, 3, or 4; L₂ is —NH—, —CH₂—, —O— or —C═C—.

19. The compound according to claim 17, wherein v=0; w=0; L₄ is —CH₂CH₂O—; z=1, 2, 3, 4, 5, 6, or 7; L₆ is —CH₂—; r=0, 1, 2, or 3; L₂ is —NH—, or —CH₂—.

20. The compound according to claim 18, wherein v=0; w=0; L₄ is —CH₂CH₂O—; z=1, 2, 3; L₆ is —CH₂—; r=1, 2, or 3; L₂ is —C═C—.

21. The compound according to claim 17, wherein V=0, L₃ is —CH₂—, w=2 or 3, L₄ is —CH₂CH₂O— or —CH₂—, z=1, 2, 3, or 4; L₆ is —CH₂—; r=1, 2, or 3; L₂ is —NH—, or —CH₂—.

22. The compound according to claim 17, wherein V=0, L₃ is —CH₂—, w=2 or 3, L₄ is z=3, 4 or 5; r=0; L₂ is 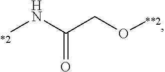
wherein *2 refers to the position attached to L₄ and **2 refers to the position attached to the Degron.

23. The compound according to claim 17, wherein the Linker is selected from 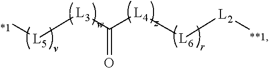
wherein
L₅ is —CH₂CH₂O—, or 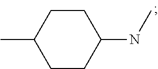
v=0, 1, 2 or 3, L₃ is —CH₂— or 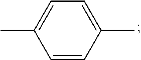
w=0, 1, 2, or 3; L₄ is —CH₂—O—CH₂—, —CH₂—, 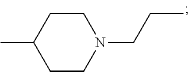
z=0, 1, 2, 3, 4, 5, or 6; L₆ is —CH₂—, —OCH₂CH₂—, 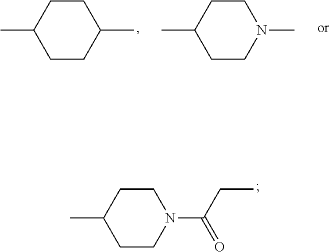
r=0, 1, 2, 3, 4, 5, 6, 7 or 8; L₂ is —NH—,

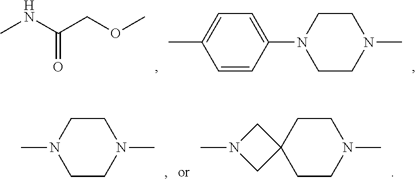

24. The compound according to claim 23, wherein L₅ is —CH₂CH₂O—; v=1, 2 or 3, L₃ is —CH₂—; w=1; z=0; r=0; L₂ is —NH—.

25. The compound according to claim 23, wherein v=w=0; L₄ is —CH₂—O—CH₂—; z=1, 2, 3 or 4; L₆ is —CH₂—; r=1, 2, 3, 4, 5, 6, 7 or 8; L₂ is —NH— or

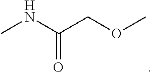

26. The compound according to claim 23, wherein v=w=z=0; L₆ is —CH₂—; r=2, 3, 4, 5, or 6; L₂ is —NH— or

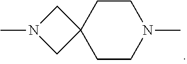

27. The PROTAC compound according to claim 23, wherein v=w=0; L₄ is 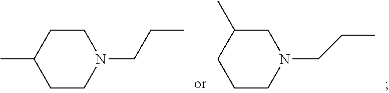
z=1; L₆ is —OCH₂CH₂—; r=1, 2, 3; L₂ is —NH—.

28. The PROTAC compound according to claim 17, wherein the Linker is selected from 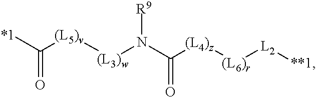
wherein
L₅ is —CH₂CH₂O—, —CH₂— or —CH₂—O—CH₂—;
v=1, 2, 3 or 4, L₃ is —CH₂—, 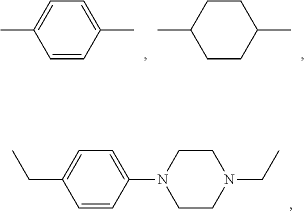
or —CH₂CH₂O—;
w=0, 1, 2 or 3;
R⁹ is H or CH₃;
L₄ is —CH₂— or —CH₂—O—CH₂—;
z=0, 1, 2, 3, or 4; L₆ is —CH₂—;
r=0, 1, 2, 3, or 4; L₂ is —NH—, —CH₂—, —O—,

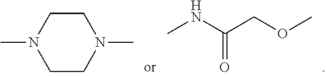

29. The PROTAC compound according to claim 28, wherein
L₅ is —CH₂—;
v=1, 2, 3 or 4,
L₃ is 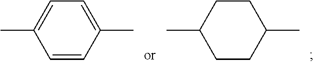
w=1;
R⁹ is H;
L₄ is —CH₂—;
z=1; r=0; L₂ is —NH—.

30. The compound according to claim 17, wherein the Linker is selected from 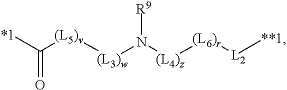
wherein
L₅ is —CH₂CH₂O—, —CH₂—, —CH═CH—, or —CH₂—O—CH₂—;
v=1, 2, 3 or 4, L₃ is —CH₂—, —C(O)— 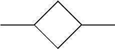
or —CH₂CH₂O—;
w=0, 1, 2, or 3; R⁹ is H or CH₃; L₄ is —CH₂CH₂O—, —CH₂—; —CH₂—O—CH₂—, —CH₂—CONH— or 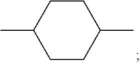
z=0, 1, 2, 3, 4, or 5, L₆ is —CH₂—, 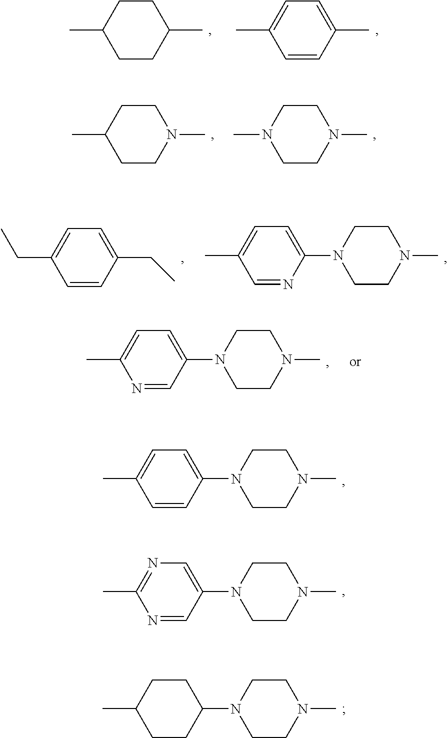
r=0, 1, 2, 3, 4, 5, or 6; L₂ is —NH—, —C(O)—, —O—,

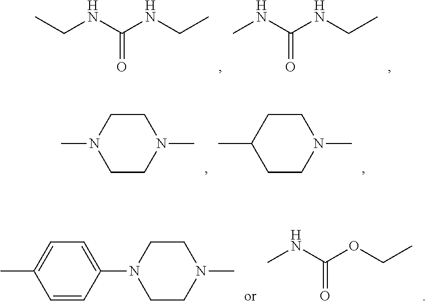

31. The compound according to claim 30, wherein L₅ is —CH₂—; v=2; w=0; R⁹ is CH₃; L₄ is —CH₂—; z=1, 2, 3, or 4; L₆ is —CH₂—, 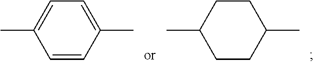
r=0, 1 or 2; L₂ is —NH—,

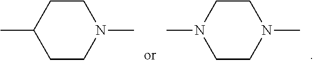

32. The compound according to claim 30, wherein L₅ is —CH═CH—; v=1, L₃ is —CH₂—; w=0 or 1; R⁹ is H or CH₃; L₄ is —CH₂CH₂O— or —CH₂—; z=1, 2, 3, 4, 5 or 6; L₆ is —CH₂—; r=0, 1, 2; L₂ is —NH—, —CH₂—, or

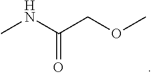

33. The compound according to claim 30, wherein v=0;
L₃ is 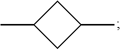
w=1;
R⁹ is CH₃;
L₄ is —CH₂—;
z=1 or 2;
L₆ is 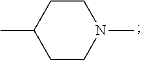
r=0 or 1;
L₂ is —NH—, 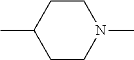
or —C(O)—.

34. The compound according to claim 17, wherein
the Linker is selected from
wherein 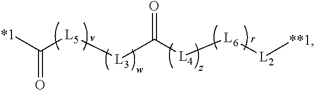
L₅ is —CH═CH—;
v=1, 2, 3 or 4; 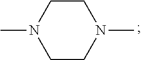
L₃ is
w=1;
L₄ is —CH₂—;
z=1, 2;
L₆ is —CH₂—;
r=0;
L₂ is —NH— or —CH₂—.

35. The compound according to claim 17, wherein the Linker is selected from 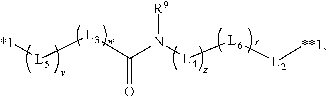
wherein
L₅ is 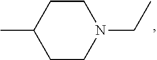
CH₂CH₂O—, —CH₂— or —CH₂—O—CH₂—;
v=1, 2, 3 or 4,
L₃ is —CH₂—, 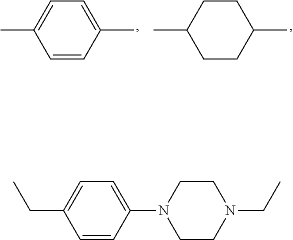
or —CH₂CH₂O—;
w=0, 1, 2 or 3;
R⁹ is H or CH₃;
L₄ is —CH₂—, —CH₂—O—CH₂—, 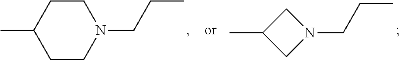
z=0; 1, 2, 3, or 4;
L₆ is —CH₂— or —OCH₂CH₂—;
r=0, 1, 2, 3, or 4;
L₂ is —NH—, —CH₂—, —O—,

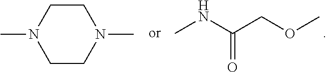

36. The compound according to claim 17, wherein
the Linker is selected from 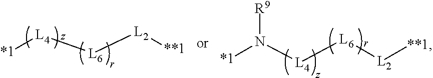
L₄ is 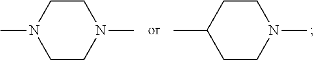
z=1;
L₆ is —CH₂—, 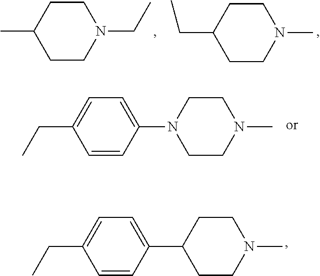
r=0 or 1;
L₂ is 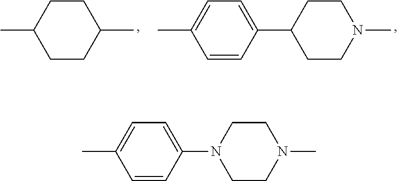
or —C(O)—;
R⁹ is H or CH₃.

37. The compound according to claim 1, wherein the Linker is selected from

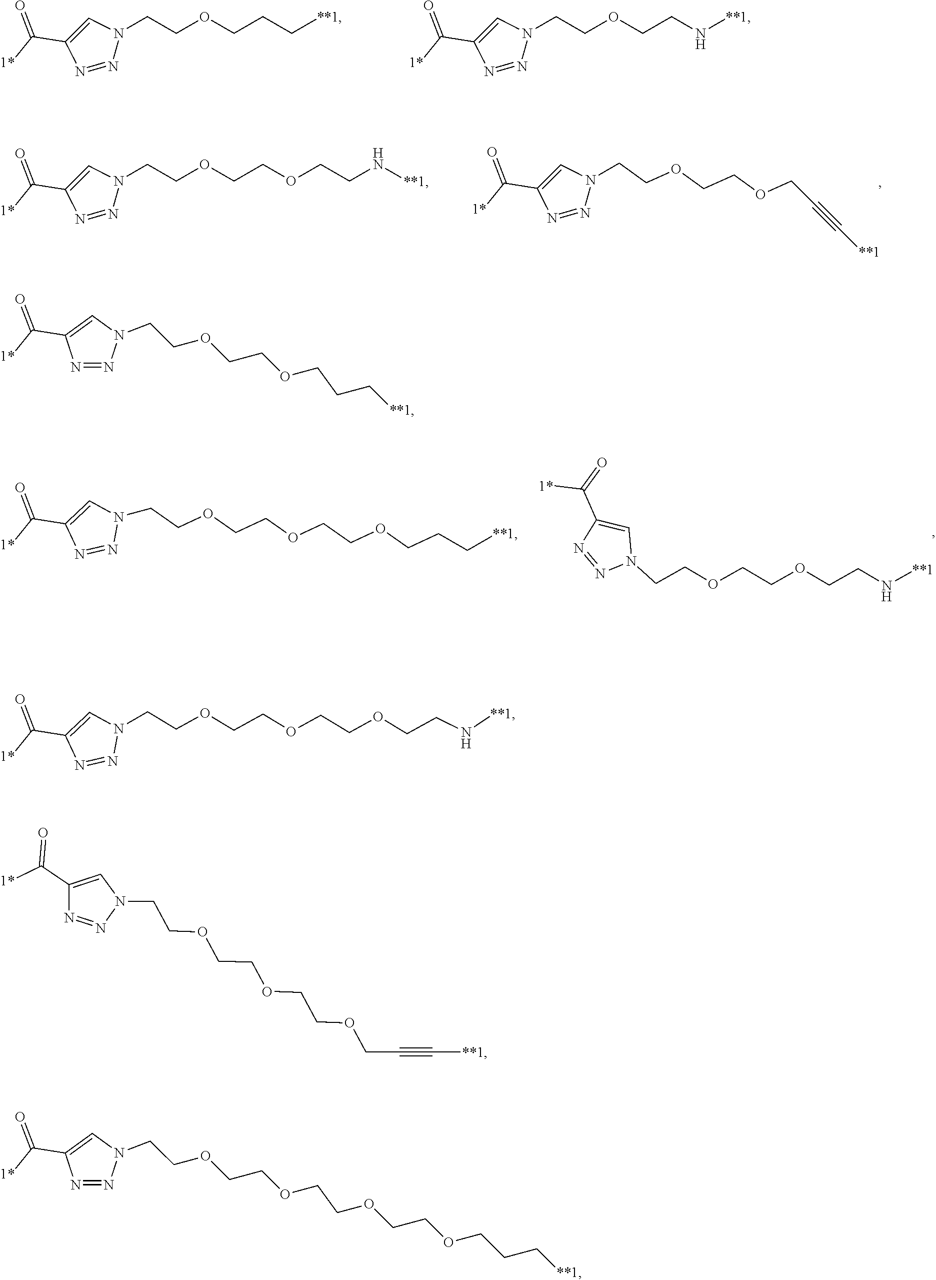

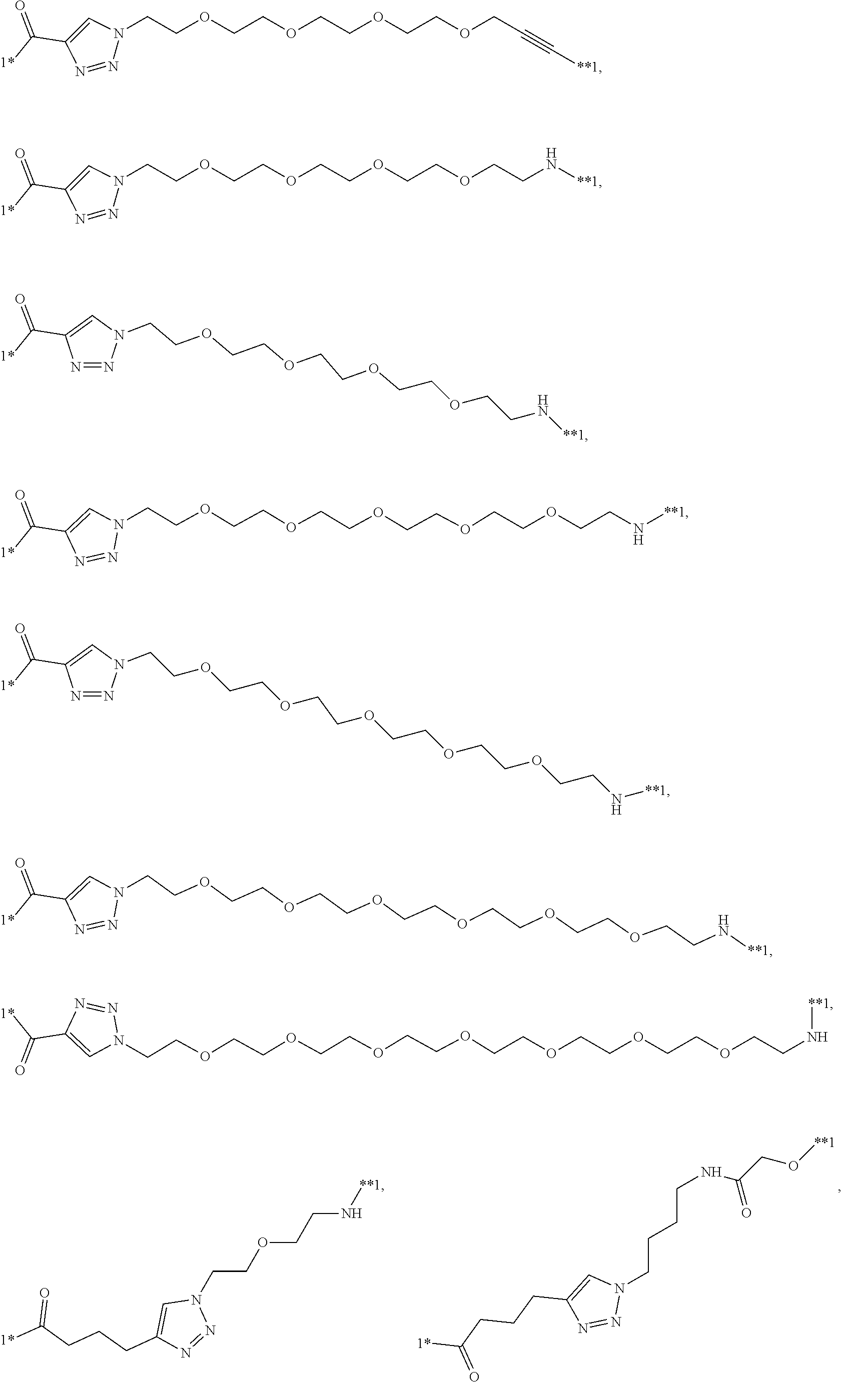

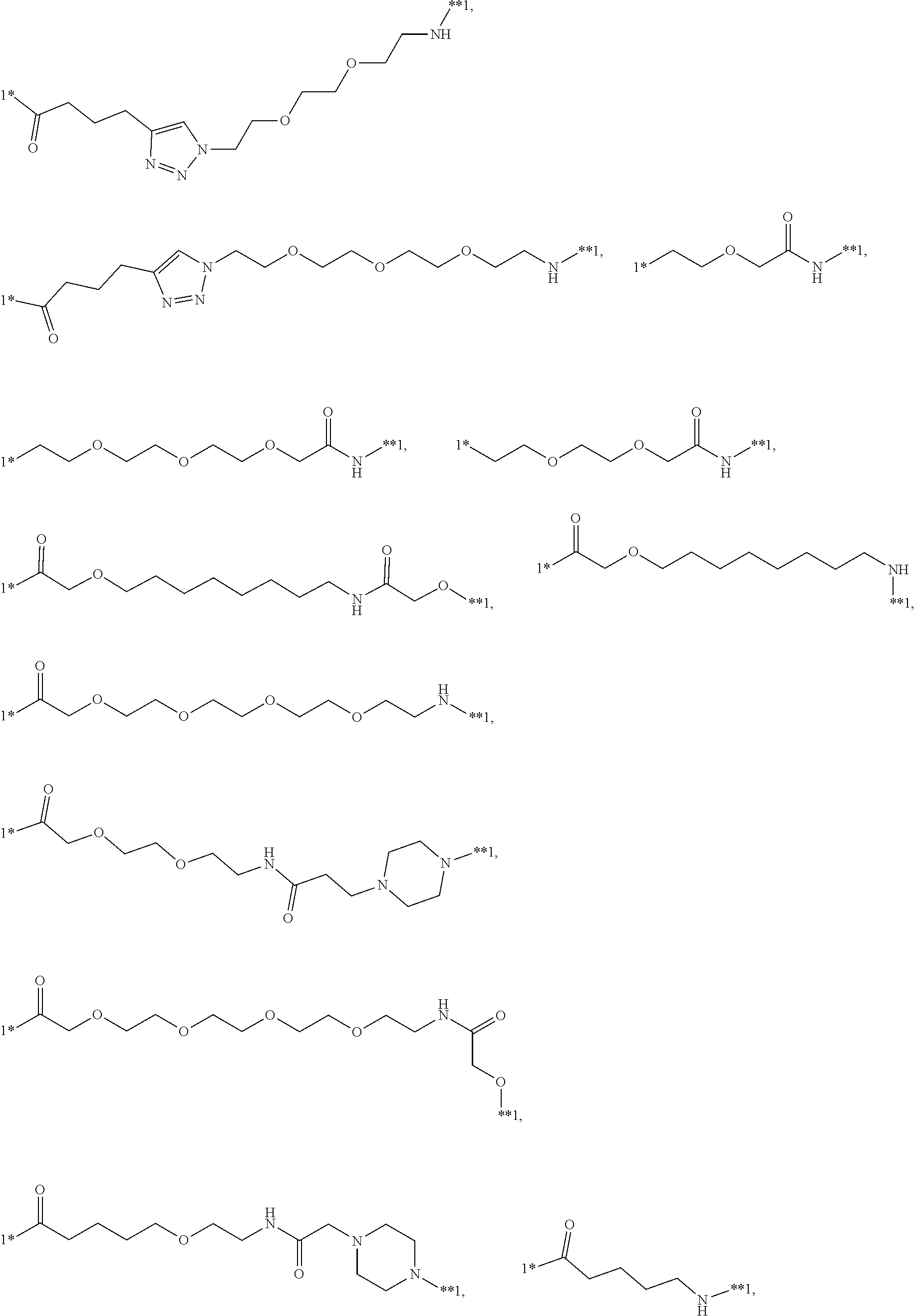

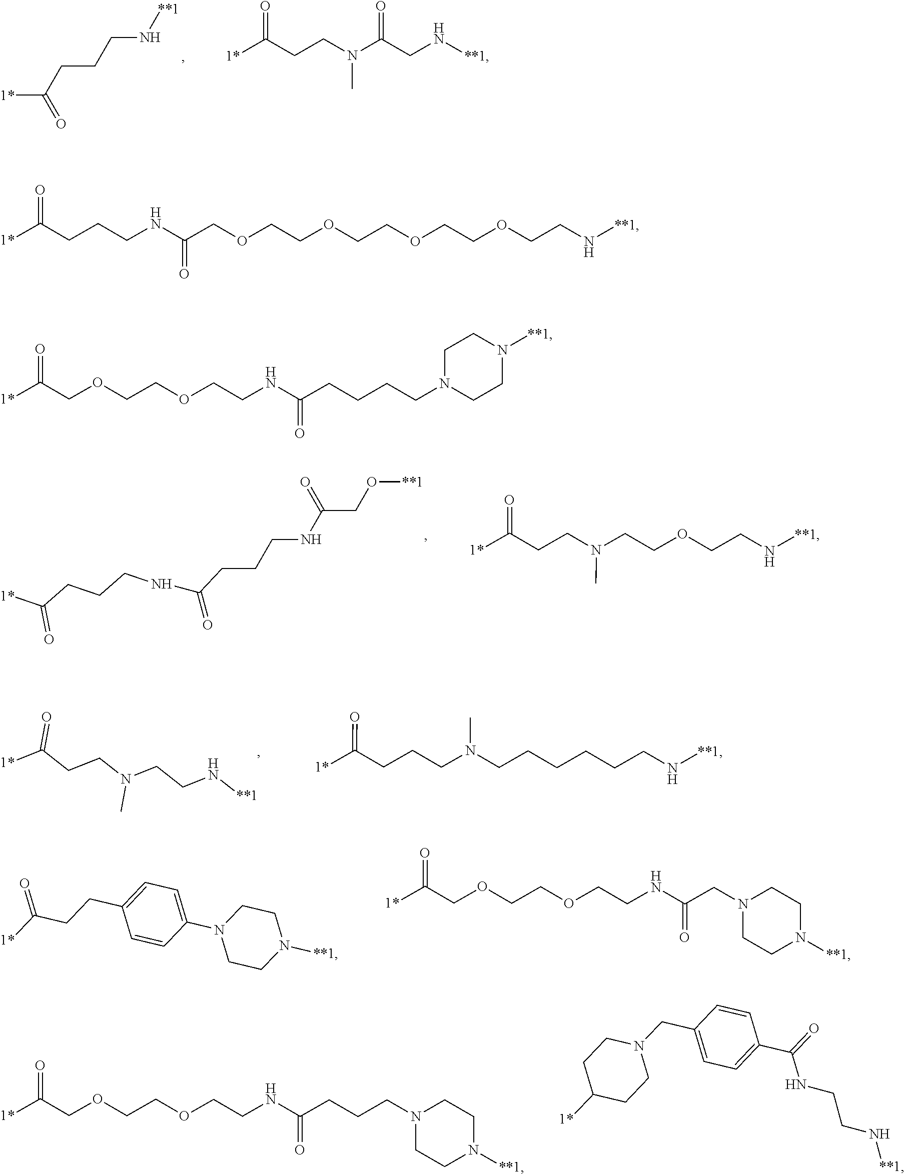

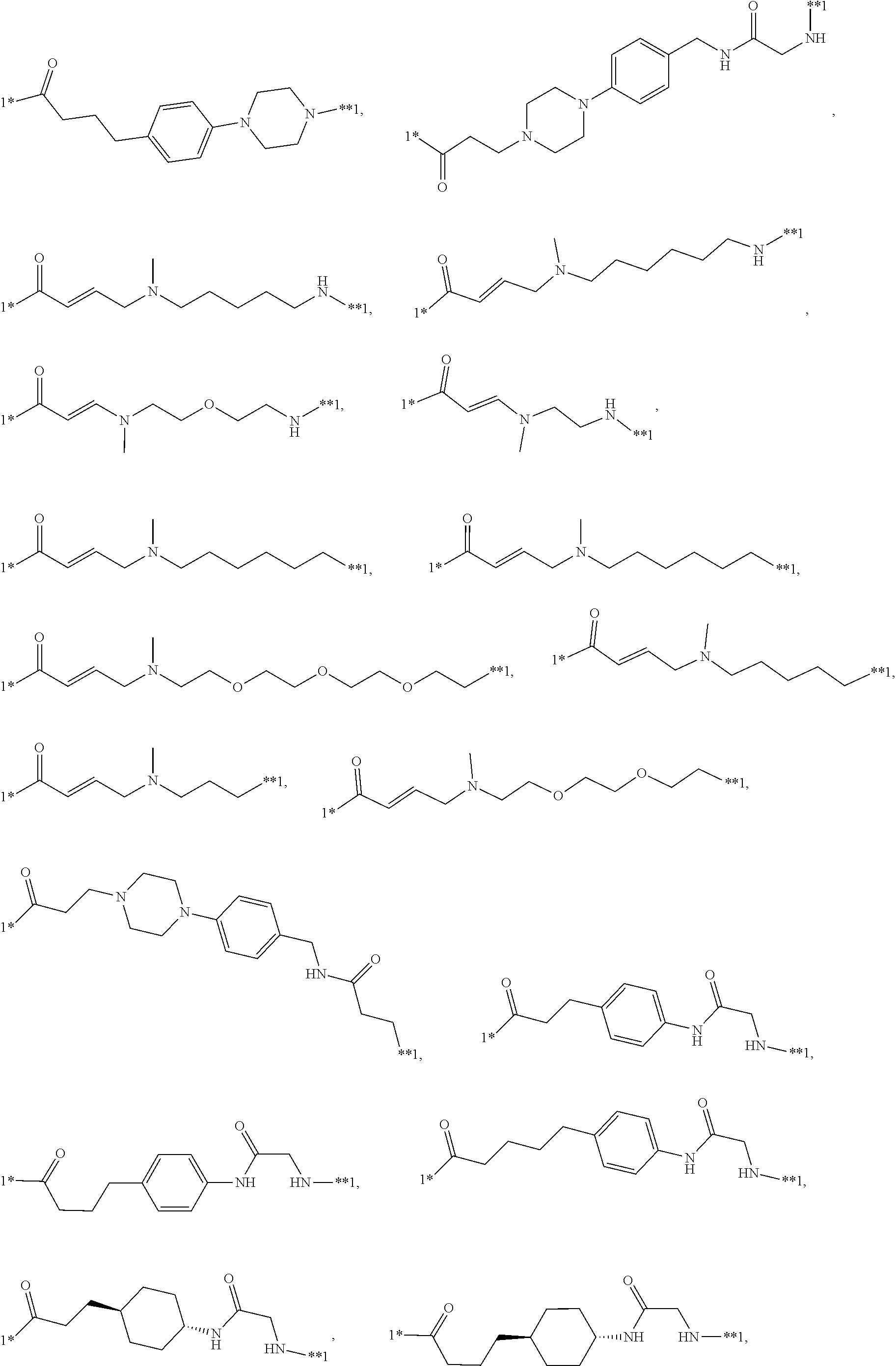

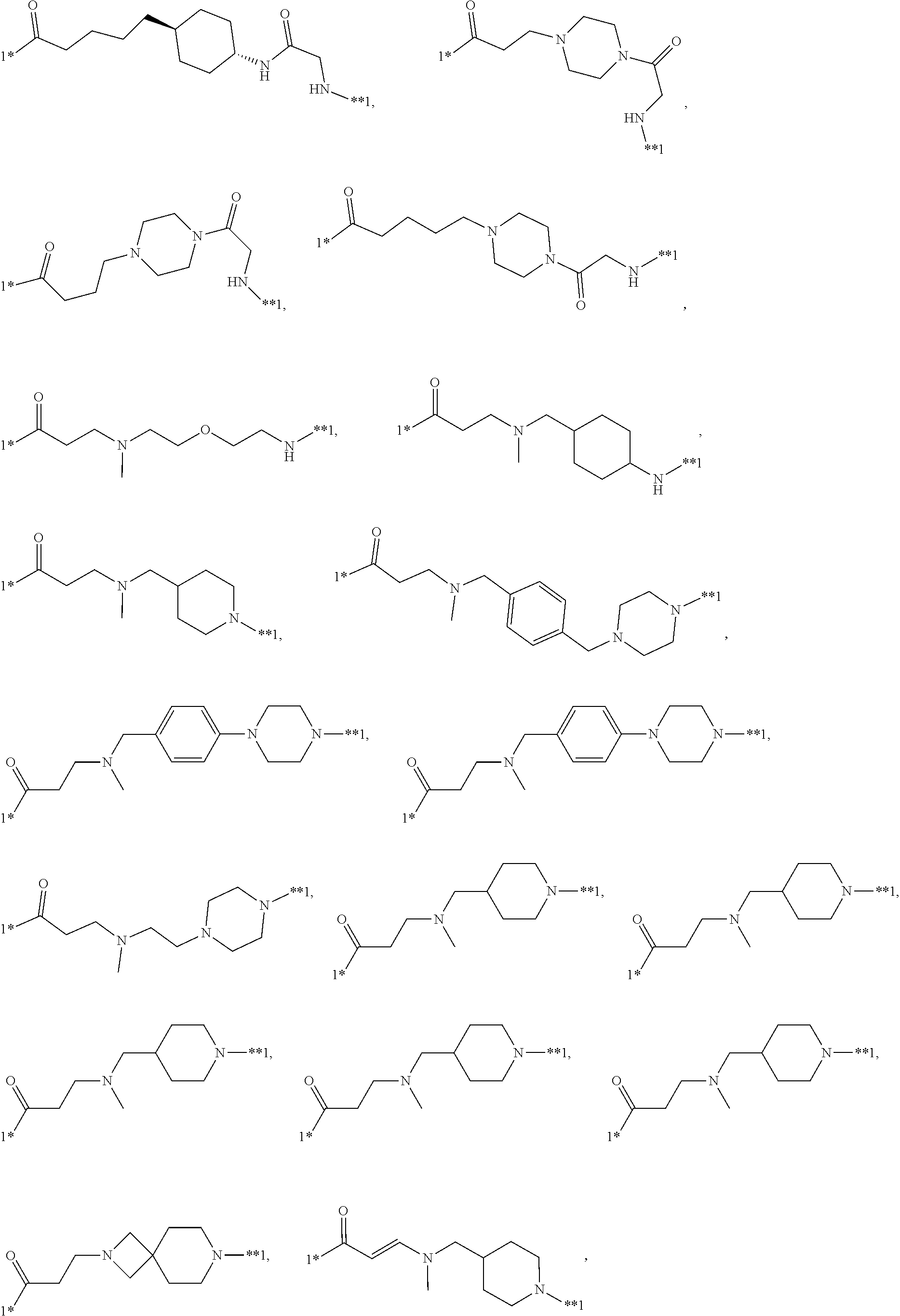

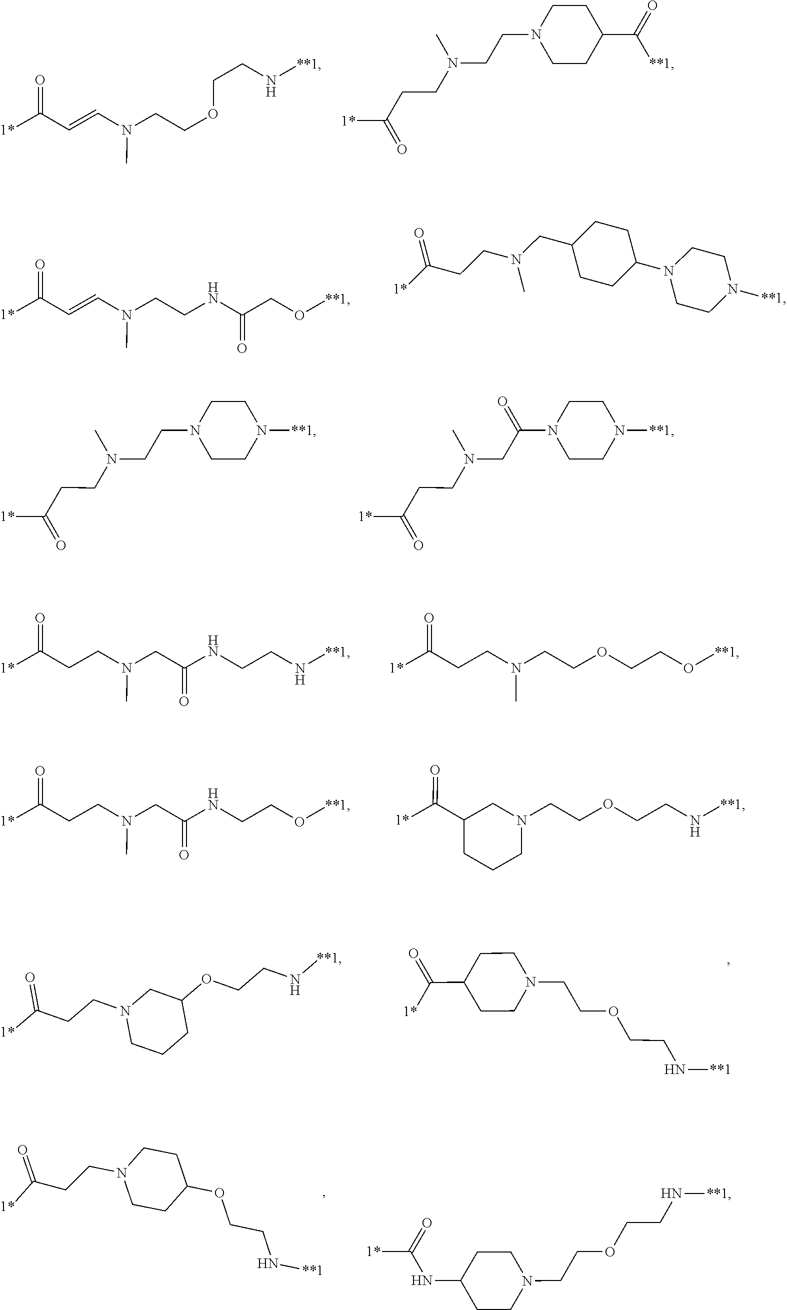

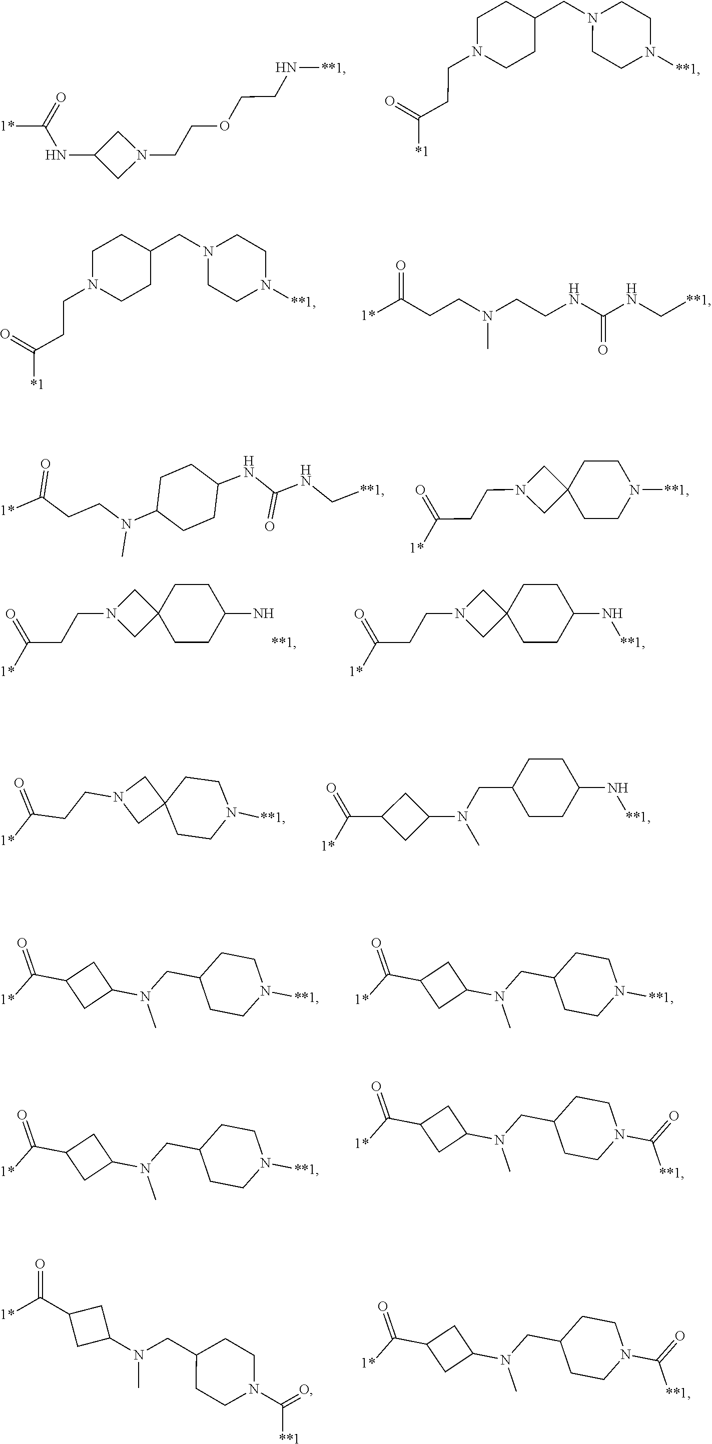

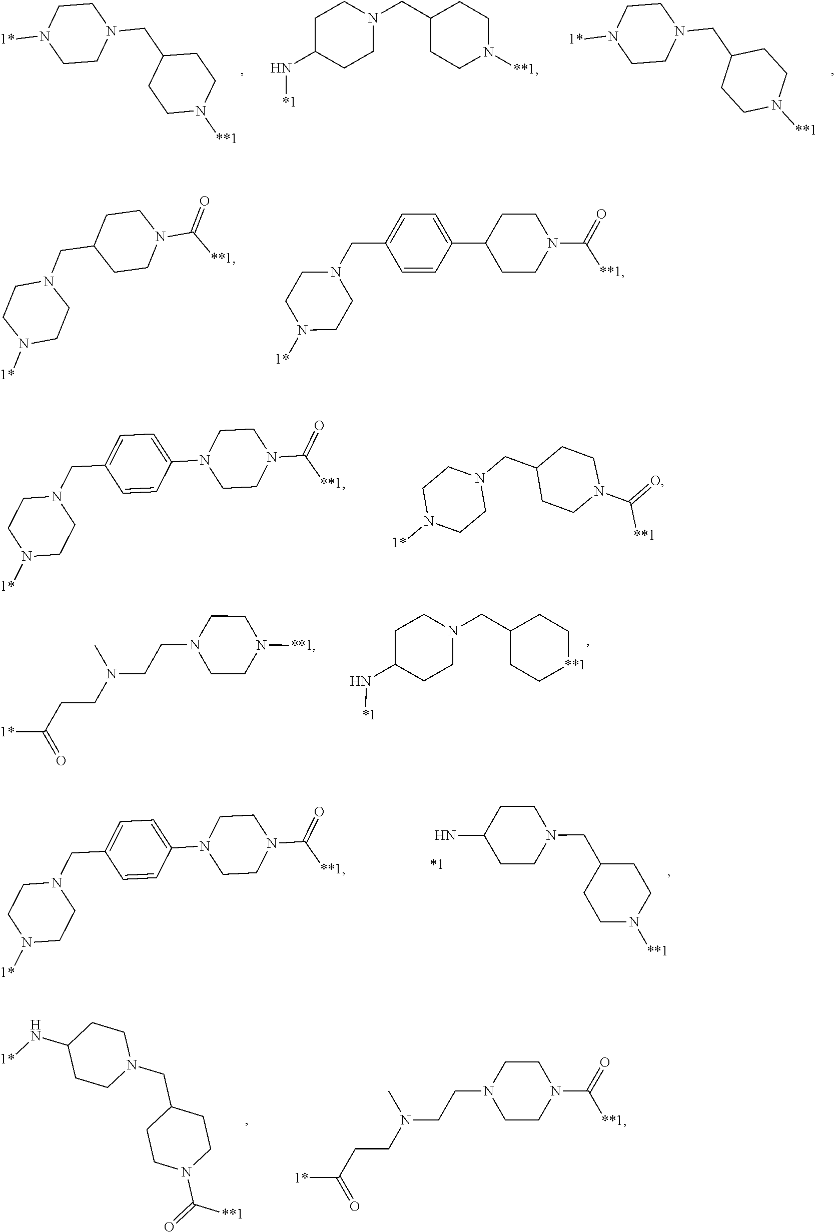

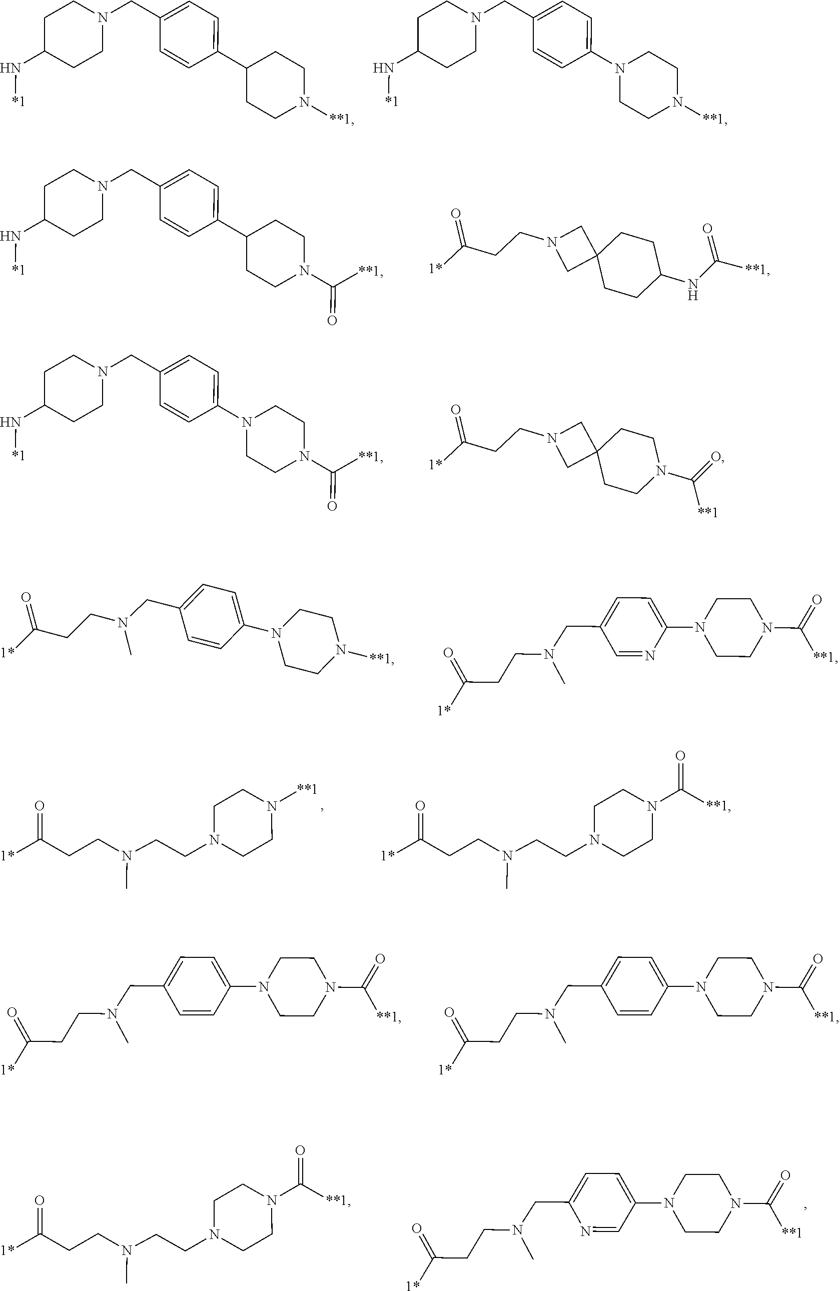

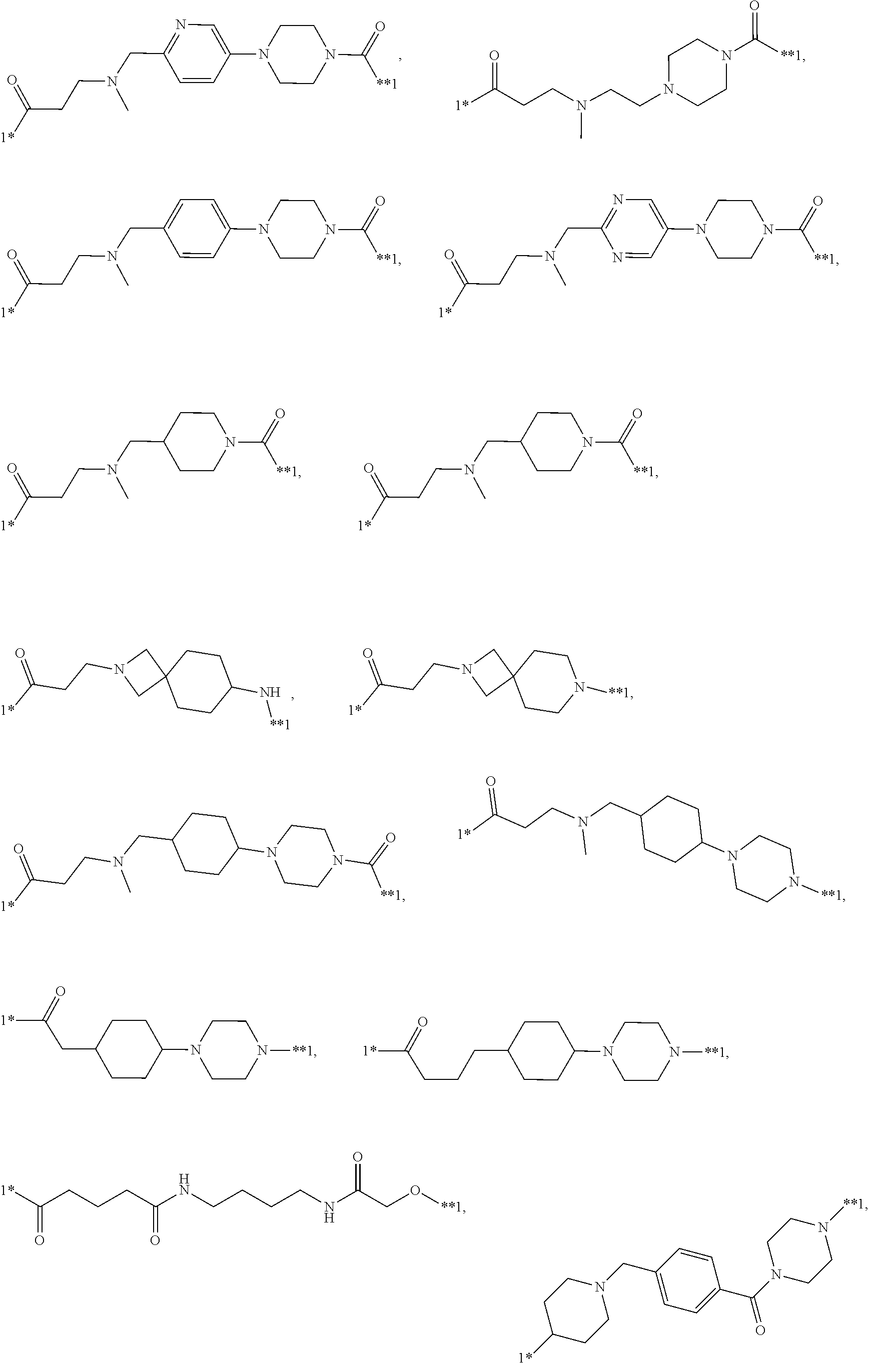

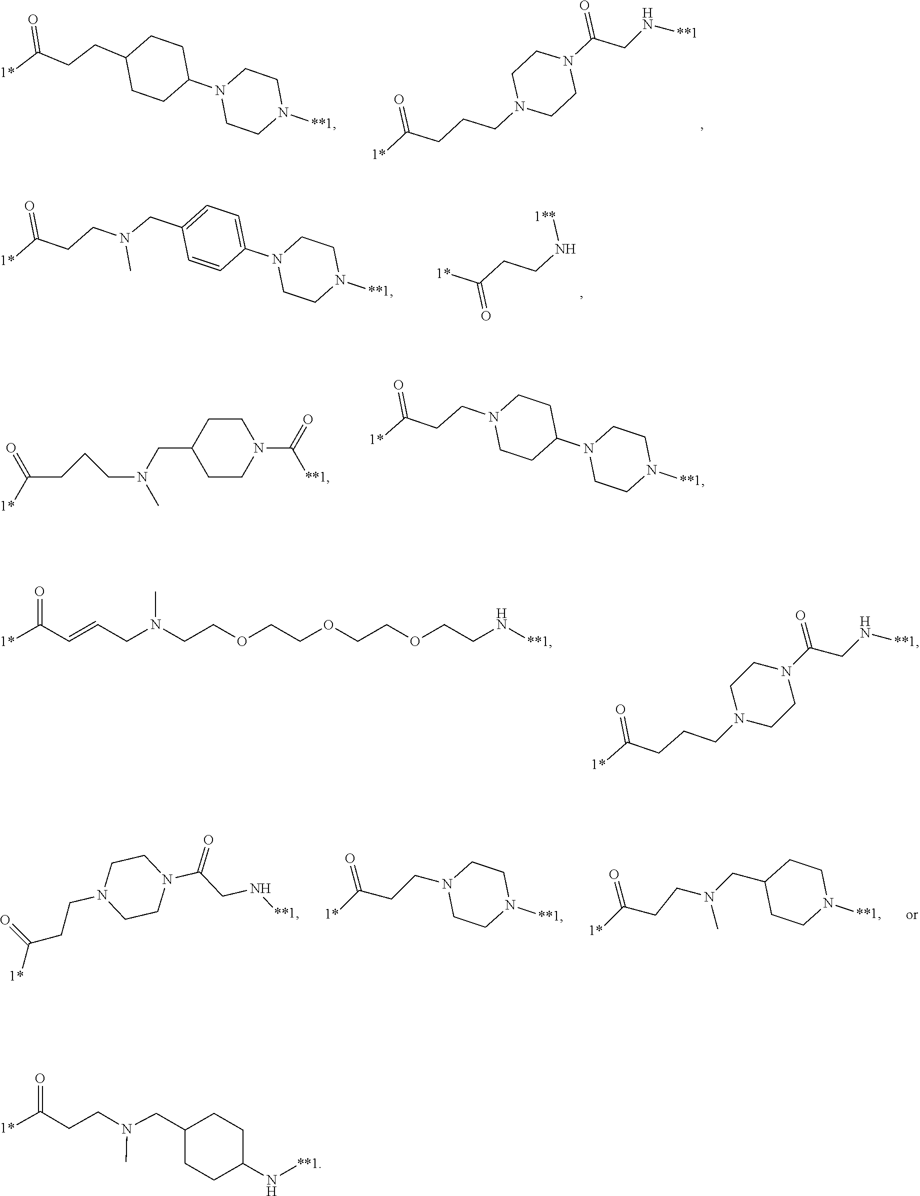

38. The compound according to claim 1, wherein the compound is selected from Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, or 159.

39. A pharmaceutical composition comprising the compound according to any one of claims 1-38, or a pharmaceutically acceptable salt thereof, and at least one pharmaceutically acceptable carrier or excipient.

40. A method of inhibiting BTK activity, which comprises administering to an individual the compound according to any one of claims 1-38, or a pharmaceutically acceptable salt thereof, including the compound of formula (I) or the specific compounds exemplified herein.

41. A method of treating a disease or disorder in a patient comprising administering to the patient a therapeutically effective amount of the compound any one of claims 1-38, or a pharmaceutically acceptable salt thereof as an BTK kinase inhibitor, wherein the disease or disorder is associated with inhibition of BTK, preferably, the disease or disorder is cancer.
